(19) United States
(12) Patent Application Publication
(10) Pub. No.: US 2023/0269958 A1
(43) Pub. Date: Aug. 24, 2023

(54) ORGANIC COMPOUND, AND ELECTRONIC COMPONENT AND ELECTRONIC DEVICE HAVING SAME

(71) Applicant: Shaanxi Lighte Optoelectronics Material Co., Ltd., Xi'an (CN)

(72) Inventors: Linnan MA, Xi'an (CN); Peng NAN, Xi'an (CN); Youngkook KIM, Xi'an (CN); Yingwen LI, Xi'an (CN)

(21) Appl. No.: 18/012,006

(22) PCT Filed: Apr. 1, 2022
(86) PCT No.: PCT/CN2022/084920

§ 371 Date: Dec. 21, 2022

(30) Foreign Application Priority Data
Apr. 9, 2021 (CN) 202110383938.6
Sep. 9, 2021 (CN) 202111056876.4

(51) Int. Cl. H10K 50/18; C07F 7/08; H10K 85/60; H10K 85/40; C07C 211/54; C07D 307/91; C07D 333/76; C07D 209/82; C07D 409/12; C07D 405/12

(52) U.S. Cl. CPC H10K 50/181; C07F 7/0812; H10K 85/631; H10K 85/40; H10K 85/633; H10K 85/6574; H10K 85/6576; C07C 211/54; C07D 307/91; C07D 333/76; C07D 209/82; H10K 85/6572; C07D 409/12; C07D 405/12; H10K 85/626; H10K 85/636

(57) ABSTRACT

The present disclosure belongs to the field of organic materials, and relates to an organic compound, and an electronic component and electronic device having same. The organic compound has a structure represented by a formula 1, and when the organic compound is applied in an organic electroluminescent device, the performance of the device can be significantly improved.

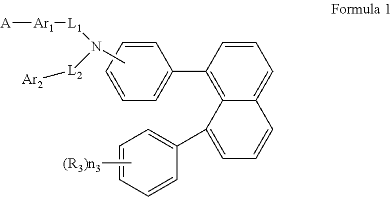

### CROSS REFERENCE TO RELATED APPLICATIONS

The present application claims the priority of Chinese Patent Application No. 202110383938.6, filed on Apr. 9, 2021, and Chinese Patent Application No. 202111056876.4, filed on Sep. 9, 2021, the contents of which are incorporated herein by reference in their entirety.

### FIELD

The present disclosure belongs to the technical field of organic materials, and specifically provides an organic compound, and an electronic component and electronic device having the same.

### BACKGROUND

With the development of an electronic technology and the progress of material science, electronic components for realizing electroluminescence or photoelectric conversion are more and more widely used. Such electronic component typically includes a cathode and an anode which are oppositely disposed, and a functional layer disposed between the cathode and the anode. The functional layer consists of a multiple of organic or inorganic film layers, and generally includes an energy conversion layer, a hole transport layer between the energy conversion layer and the anode, and an electron transport layer between the energy conversion layer and the cathode.

Taking an organic electroluminescent device as an example, the organic electroluminescent device generally includes an anode, a hole transport layer, an electroluminescent layer serving as an energy conversion layer, an electron transport layer, and a cathode which are sequentially stacked. When a voltage is applied to the cathode and the anode, an electric field is generated between the two electrodes, under the action of the electric field, the electrons on the cathode side move to the electroluminescent layer, while the holes on an anode side move to the electroluminescent layer, so the electrons and the holes are combined in the electroluminescent layer to form excitons, and the excitons are in an excited state and release energy outwards, which in turn makes the electroluminescent layer emit light outward.

In an organic light-emitting device structure, an electron blocking layer is used to block electrons transported from an organic light-emitting layer, thus ensuring that electrons and holes can be recombined very efficiently in the organic light-emitting layer; and at the same time, the electron blocking layer can also block excitons diffused from the organic light-emitting layer, reducing triplet state quenching of the excitons, thus ensuring the luminous efficiency of the organic electroluminescent device. A material of the electron blocking layer has a relatively high LUMO value, which can effectively block the transport and diffusion of electrons and excitons from the organic light-emitting layer to the anode. With the continuous development of the market, the requirements for the luminous efficiency, service life and other properties of devices are becoming higher and higher, and developing stable and efficient electron blocking layer materials, thus reducing the driving voltage, improving the luminous efficiency of the device, and prolonging the service life of the device, has a very important practical application value.

### SUMMARY

The present disclosure aims to provide an organic compound, and an electronic component and electronic device having same. When the organic compound is used in an electronic component, the performance of the electronic component can be improved.

In a first aspect, the present disclosure provides an organic compound, having a structure represented by a formula 1:

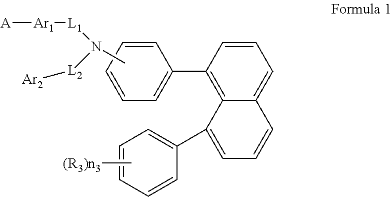

in the formula 1, A is selected from adamantyl, norbornyl, or cyclohexyl;

Ar₁ is selected from substituted or unsubstituted aryl with 6 to 40 carbon atoms, and substituted or unsubstituted heteroaryl with 3 to 30 carbon atoms;

Ar₂ is selected from

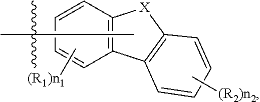

where X is selected from C(R₄R₅), N(R₆), O, S, or Si(R₇R₈), and [symbol] represents a chemical bond;

R₄, R₅, R₆, R₇ and R₈ are the same or different, and are each independently selected from hydrogen, alkyl with 1 to 5 carbon atoms, aryl with 6 to 12 carbon atoms, aryl with 7 to 17 carbon atoms substituted with alkyl with 1 to 5 carbon atoms, and heteroaryl with 3 to 12 carbon atoms; or, R₄ and R₅ form a saturated or unsaturated 3- to 15-membered ring; or, R₇ and R₈ form a saturated or unsaturated 3- to 15-membered ring;

R₁, R₂ and R₃ are the same or different, and are each independently selected from deuterium, cyano, a halogen group, alkyl with 1 to 5 carbon atoms, aryl with 6 to 12 carbon atoms, heteroaryl with 3 to 12 carbon atoms, and trialkylsilyl with 3 to 12 carbon atoms;

n₁ represents the number of R₁, n₂ represents the number of R₂, n₃ represents the number of R₃, n₁ and n₂ are each independently selected from 0, 1, 2, 3 or 4, and n₃ is selected from 0, 1, 2, 3, 4 or 5; and when n₁ is greater than 1, any two R₁ are the same or different; when n₂ is greater than 1, any two R₂ are the same or different; and when n₃ is greater than 1, any two R₃ are the same or different;

L₁ and L₂ are the same or different, and are each independently selected from a single bond, substituted or unsubstituted arylene with 6 to 30 carbon atoms, and substituted or unsubstituted heteroarylene with 3 to 30 carbon atoms; and

substituents in L₁, L₂, and Ar₁ are the same or different, and are respectively and independently selected from deuterium, cyano, a halogen group, alkyl with 1 to 5 carbon atoms, aryl with 6 to 12 carbon atoms, heteroaryl with 3 to 12 carbon atoms, and trialkylsilyl with 3 to 12 carbon atoms.

Optionally, R₄, R₅, R₆, R₇, and R₈ are the same or different, and are each independently selected from alkyl with 1 to 5 carbon atoms, aryl with 6 to 12 carbon atoms, aryl with 7 to 17 carbon atoms substituted with alkyl with 1 to 5 carbon atoms, and heteroaryl with 3 to 12 carbon atoms; or, R₄ and R₅ form a saturated or unsaturated 3- to 15-membered ring; or, R₇ and R₈ form a saturated or unsaturated 3- to 15-membered ring.

The organic compound of the present disclosure is a triarylamine structure including 1,8-diphenylnaphthalene group, cycloalkane, and a dibenzo five-membered ring at the same time, in this structure, the 1,8-diphenylnaphthalene group has a better electron blocking ability, and the triarylamine can increase conjugation of the molecule, effectively improving the efficiency while enhancing the film-forming properties of the molecule, and in addition, the cycloalkane structure with large steric hindrance effectively improves the stacking effect of the molecule, and increases the rigidity and thermal stability of the molecule as a whole, thus increasing the service life of an organic electroluminescent device.

In a second aspect, the present disclosure provides an electronic component, including an anode and a cathode which are oppositely disposed, and a functional layer disposed between the anode and the cathode; and the functional layer includes the organic compound described above.

In a third aspect, the present disclosure provides an electronic device, including the electronic component described above.

Other features and advantages of the present disclosure will be described in detail in the subsequent specific implementation part.

### BRIEF DESCRIPTION OF THE DRAWINGS

The drawings are used to provide a further understanding of the present disclosure, and constitute a part of the description, and are used to explain the present disclosure together with the following specific examples, but do not constitute limitations to the present disclosure.

FIG. 1 is a structural schematic diagram of an organic electroluminescent device according to one example of the present disclosure.

FIG. 2 is a schematic diagram of an electronic device according to one example of the present disclosure.

### DESCRIPTION OF REFERENCE SIGNS

100, anode; 200, cathode; 300, functional layer; 310, hole injection layer; 321, hole transport layer; 322, electron blocking layer; 330, organic light-emitting layer; 341, hole blocking layer; 340, electron transport layer; 350, electron injection layer; and 400, electronic device.

### DETAILED DESCRIPTION

The specific examples will now be described in detail below in combination with the drawings. However, the examples can be implemented in various forms and should not be construed as limited to the examples set forth here; rather, these examples are provided so that the present disclosure will be thorough and complete, and the concept of the examples is fully conveyed to those skilled in the art. The described features, structures, or characteristics may be combined in any suitable manner in one or more examples. In the following description, numerous specific details are provided to give a thorough understanding of the examples of the present disclosure.

In a first aspect, the present disclosure provides an organic compound, having a structure represented by a formula 1:

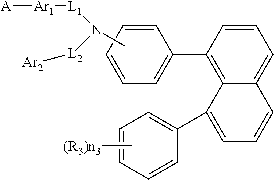

### Formula 1

in the formula 1, A is selected from adamantyl, norbornyl, or cyclohexyl;

Ar₁ is selected from substituted or unsubstituted aryl with 6 to 40 carbon atoms, and substituted or unsubstituted heteroaryl with 3 to 30 carbon atoms;

Ar₂ is selected from

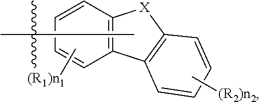

where X is selected from C(R₄R₅), N(R₆), O, S, or Si(R₇R₈), and [symbol] represents a chemical bond;

R₄, R₅, R₆, R₇ and R₈ are the same or different, and are each independently selected from hydrogen, alkyl with 1 to 5 carbon atoms, aryl with 6 to 12 carbon atoms, aryl with 7 to 17 carbon atoms substituted with alkyl with 1 to 5 carbon atoms, and heteroaryl with 3 to 12 carbon atoms; or, R₄ and R₅ form a saturated or unsaturated 3- to 15-membered ring; or, R₇ and R₈ form a saturated or unsaturated 3- to 15-membered ring, for example, the ring is cyclopentane, cyclohexane, a fluorene ring or the like;

R₁, R₂ and R₃ are the same or different, and are each independently selected from deuterium, cyano, a halogen group, alkyl with 1 to 5 carbon atoms, aryl with 6 to 12 carbon atoms, heteroaryl with 3 to 12 carbon atoms, and trialkylsilyl with 3 to 12 carbon atoms;

n₁ represents the number of R₁, n₂ represents the number of R₂, n₃ represents the number of R₃, n₁ and n₂ are each independently selected from 0, 1, 2, 3 or 4, and n₃ is selected from 0, 1, 2, 3, 4 or 5; and when n₁ is greater than 1, any two R₁ are the same or different; when n₂ is greater than 1, any two R₂ are the same or different; and when n₃ is greater than 1, any two R₃ are the same or different;

L₁ and L₂ are the same or different, and are each independently selected from a single bond, substituted or unsubstituted arylene with 6 to 30 carbon atoms, and substituted or unsubstituted heteroarylene with 3 to 30 carbon atoms; and

substituents in L₁, L₂, and Ar₁ are the same or different, and are respectively and independently selected from deuterium, cyano, a halogen group, alkyl with 1 to 5 carbon atoms, aryl with 6 to 12 carbon atoms, heteroaryl with 3 to 12 carbon atoms, and trialkylsilyl with 3 to 12 carbon atoms.

Optionally, R₄, R₅, R₆, R₇ and R₈ are the same or different, and are each independently selected from alkyl with 1 to 5 carbon atoms, aryl with 6 to 12 carbon atoms, aryl with 7 to 17 carbon atoms substituted with alkyl with 1 to 5 carbon atoms, and heteroaryl with 3 to 12 carbon atoms; or, R₄ and R₅ form a saturated or unsaturated 3- to 15-membered ring; or, R₇ and R₈ form a saturated or unsaturated 3- to 15-membered ring.

In the present disclosure, A is unsubstituted adamantyl, unsubstituted norbornyl, or unsubstituted cyclohexyl.

In the present disclosure,

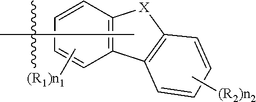

includes

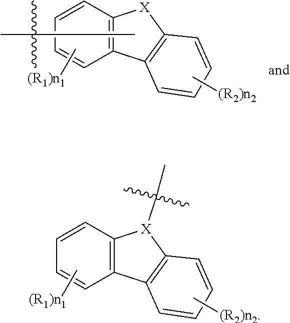

In the present disclosure, the used descriptions modes “ . . . are each independently”, “ . . . are respectively and independently” and “ . . . are independently selected from” can be interchanged, which should be understood in a broad sense, and may mean that specific options expressed by a same symbol in different groups do not influence each other, or may also mean that specific options expressed by a same symbol in a same group do not influence each other. For example, the meaning of “

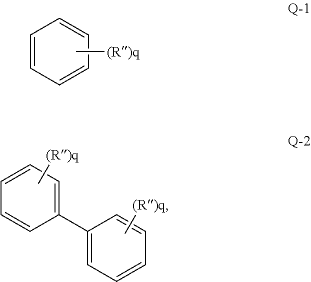

where each q is independently 0, 1, 2 or 3 and each R″ is independently selected from hydrogen, deuterium, fluorine, and chlorine” is as follows: a formula Q-1 indicates that a benzene ring has q substituents R″, each R″ can be the same or different, and the options of each R″ do not influence each other; and a formula Q-2 indicates that every benzene ring of biphenyl has q substituents R″, the number q of the substituents R″ on the two benzene rings can be the same or different, each R″ can be the same or different, and the options of each R″ do not influence each other.

In the present disclosure, the term “substituted or unsubstituted” means that a functional group defined by the term may or may not have substituents (the substituents are collectively referred to as Rc below for ease of description). For example, “substituted or unsubstituted aryl” refers to aryl with a substituent Rc or unsubstituted aryl. The above substituent, i.e. Rc, can be, for example, deuterium, cyano, a halogen group, alkyl, aryl, heteroaryl, or trialkylsilyl.

In the present disclosure, the number of carbon atoms of a substituted or unsubstituted functional group refers to the number of all carbon atoms. For example, if the L₂ is selected from substituted arylene with 12 carbon atoms, the number of all carbon atoms of the arylene and substituents on the arylene is 12. For example: if Ar₁ is

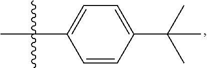 [SMILES: *c1ccc(C(C)(C)C)cc1]

then the number of carbon atoms is 10; and if L₂ is

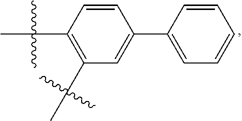 [SMILES: *c1ccc(-c2ccccc2)cc1*]

the number of carbon atoms is 12. In addition, A represents a group connected to Ar₁, and when Ar₁ is unsubstituted aryl (heteroaryl), A is directly connected to the aryl (heteroaryl), and when Ar₁ is substituted aryl (heteroaryl) (a substituent is Rc), Ar₁ may be connected to the aryl (heteroaryl), and may also be connected to the substituent Rc, and preferably, Ar₁ is directly connected to the aryl (heteroaryl). “Unsubstituted aryl (heteroaryl)” means unsubstituted aryl or unsubstituted heteroaryl, and “substituted aryl (heteroaryl)” means substituted aryl or substituted heteroaryl.

In the present disclosure, “alkyl” may include linear alkyl or branched alkyl. The alkyl may have 1 to 5 carbon atoms, and in the present disclosure, a numerical range such as “1 to 5” refers to each integer in a given range; for example, “alkyl with 1 to 5 carbon atoms” refers to alkyl containing 1 carbon atom, 2 carbon atoms, 3 carbon atoms, 4 carbon atoms, or 5 carbon atoms, and specific examples include, but are not limited to, methyl, ethyl, propyl, isopropyl, n-butyl, isobutyl, sec-butyl, tert-butyl, and pentyl.

In the present disclosure, aryl refers to an optional functional group or substituent derived from an aromatic carbon ring. The aryl can be monocyclic aryl (e.g., phenyl) or polycyclic aryl, in other words, the aryl can be monocyclic aryl, fused aryl, two or more monocyclic aryl conjugatedly linked by carbon-carbon bonds, monocyclic aryl and fused aryl which are conjugatedly linked by a carbon-carbon bond, and two or more fused aryl conjugatedly linked by carbon-carbon bonds. That is, unless specified otherwise, two or more aromatic groups conjugatedly linked by carbon-carbon bonds can also be regarded as aryl of the present disclosure. The fused aryl may, for example, include bicyclic fused aryl (e.g., naphthyl), tricyclic fused aryl (e.g., phenanthryl, fluorenyl, and anthryl), and the like. The aryl does not contain heteroatoms such as B, N, O, S, P, Se and Si. For example, in the present disclosure, biphenyl, terphenyl and the like are aryl. Examples of the aryl can include, but are not limited to, phenyl, naphthyl, fluorenyl, anthryl, phenanthryl, biphenyl, terphenyl, benzo[9,10]phenanthryl, pyrenyl, benzofluoranthenyl, chrysenyl and the like. In the present disclosure, involved arylene refers to a divalent group formed by further loss of one hydrogen atom of the aryl.

In the present disclosure, substituted aryl can be that one or two or more hydrogen atoms in the aryl are substituted with groups such as a deuterium atom, a halogen group, cyano, aryl, heteroaryl, trialkylsilyl, alkyl, cycloalkyl, haloalkyl and the like. Specific examples of heteroaryl-substituted aryl include, but are not limited to, dibenzofuranyl-substituted phenyl, dibenzothiophenyl-substituted phenyl, pyridyl-substituted phenyl and the like. It should be understood that the number of carbon atoms of the substituted aryl refers to the total number of carbon atoms of the aryl and substituents on the aryl, for example, substituted aryl with 18 carbon atoms means that the total number of carbon atoms of the aryl and substituents is 18.

In the present disclosure, heteroaryl refers to a monovalent aromatic ring containing at least one heteroatom in the ring or its derivative, and the heteroatom can be at least one of B, O, N, P, Si, Se and S. The heteroaryl may be monocyclic heteroaryl or polycyclic heteroaryl, in other words, the heteroaryl may be a single aromatic ring system or a plurality of aromatic ring systems conjugatedly connected through carbon-carbon bonds, and any one aromatic ring system is one aromatic monocyclic ring or one aromatic fused ring. For example, the heteroaryl may include thienyl, furyl, pyrrolyl, imidazolyl, thiazolyl, oxazolyl, oxadiazolyl, triazolyl, pyridyl, bipyridyl, pyrimidinyl, triazinyl, acridinyl, pyridazinyl, pyrazinyl, quinolyl, quinazolinyl, quinoxalinyl, phenoxazinyl, phthalazinyl, pyridopyrimidinyl, pyridopyrazinyl, pyrazinopyrazinyl, isoquinolyl, indolyl, carbazolyl, benzoxazolyl, benzimidazolyl, benzothiazolyl, benzocarbazolyl, benzothienyl, dibenzothienyl, thienothienyl, benzofuranyl, phenanthrolinyl, isoxazolyl, thiadiazolyl, phenothiazinyl, silafluorenyl, dibenzofuranyl, as well as N-phenylcarbazolyl, N-pyridylcarbazolyl, N-methylcarbazolyl and the like, but is not limited to this. Thienyl, furyl, phenanthrolinyl, etc. are heteroaryl of the single aromatic ring system, and N-phenylcarbazolyl, and N-pyridylcarbazolyl are heteroaryl of the plurality of aromatic ring systems conjugatedly connected through carbon-carbon bonds. In the present disclosure, involved heteroarylene refers to a divalent group formed by further loss of one hydrogen atom of the heteroaryl.

In the present disclosure, substituted heteroaryl can be that one or two or more hydrogen atoms in the heteroaryl are substituted with groups such as a deuterium atom, a halogen group, cyano, aryl, heteroaryl, trialkylsilyl, alkyl, cycloalkyl, haloalkyl and the like. Specific examples of aryl-substituted heteroaryl include, but are not limited to, phenyl-substituted dibenzofuranyl, phenyl-substituted dibenzothienyl, phenyl-substituted pyridyl and the like. It should be understood that the number of carbon atoms of the substituted heteroaryl refers to the total number of carbon atoms of the heteroaryl and substituents on the heteroaryl.

In the present disclosure, the number of carbon atoms of the aryl as a substituent can be 6 to 12, for example, the number of carbon atoms can be 6, 7, 8, 9, 10, 11, or 12, and specific examples of the aryl as the substituent include, but are not limited to, phenyl, naphthyl, biphenyl and the like.

In the present disclosure, the number of carbon atoms of the heteroaryl as a substituent can be 3 to 12, for example, the number of carbon atoms can be 3, 4, 5, 6, 7, 8, 9, 10, 11, or 12, and specific examples of the heteroaryl as the substituent include, but are not limited to, pyridyl, pyrimidinyl, carbazolyl, dibenzofuranyl, dibenzothienyl, quinolyl, quinazolinyl, and quinoxalinyl.

In the present disclosure, the halogen group may include fluorine, iodine, bromine, chlorine, and the like.

In the present disclosure, specific examples of trialkylsilyl include, but are not limited to, trimethylsilyl, triethylsilyl and the like.

In the present disclosure, spirofluorenyl may be spirobifluorenyl.

In the present disclosure, an unpositioned connecting bond refers to a single bond “ [symbol] ” extending from a ring system, which indicates that one end of the connecting bond can be connected to any position in the ring system through which the bond penetrates, and the other end of the connecting bond is connected to the remaining part of a compound molecule.

For example, as shown in a formula (f) below, naphthyl represented by the formula (f) is connected to other positions of a molecule by two unpositioned connecting bonds penetrating a bicyclic ring, and its meaning includes any one possible connection mode represented by formulae (f-1) to (f-10):

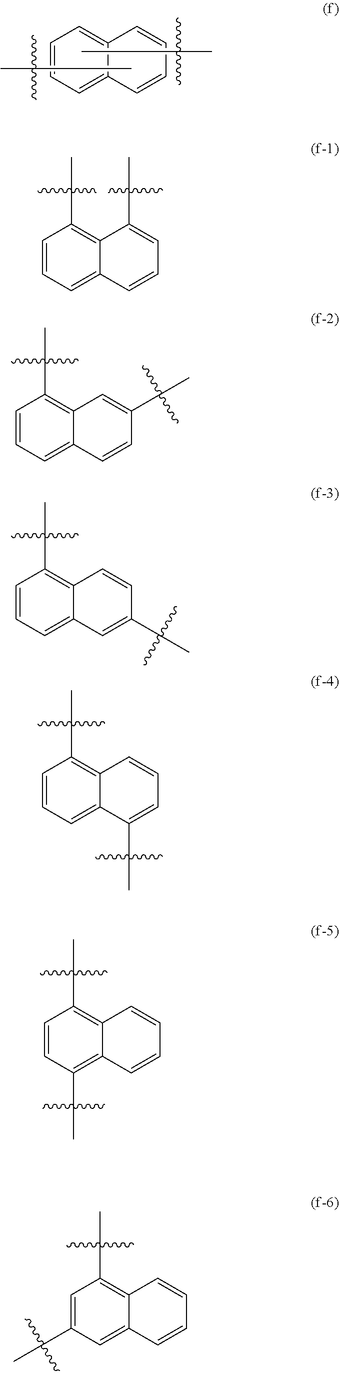

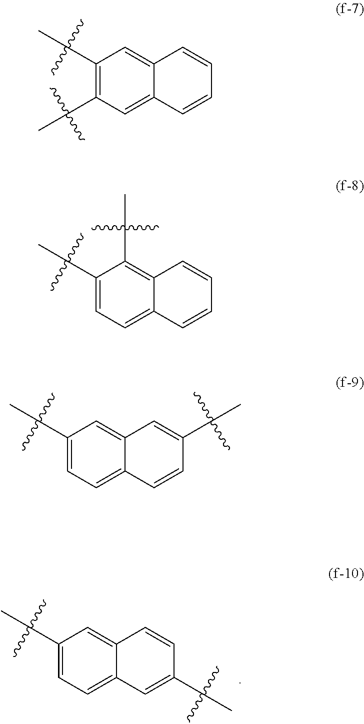 [SMILES: *c1cc2ccccc2cc1* ; *c1ccc2ccccc2c1* ; *c1ccc2ccc(*)cc2c1 ; *c1ccc2cc(*)ccc2c1]

For another example, as shown in formula (X′) below, dibenzofuranyl represented by the formula (X′) is connected to other positions of a molecule via one unpositioned connecting bond extending from the middle of a benzene ring on one side, and its meaning includes any one possible connection mode represented by formulae (X′-1) to (X′-4):

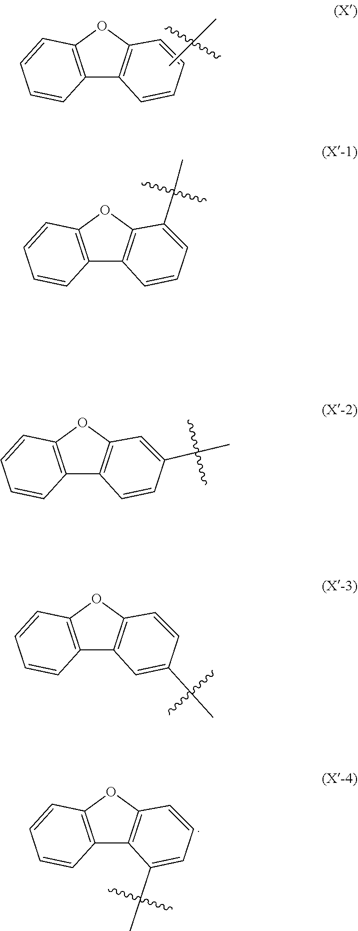

In one example of the present disclosure, Ar₁ is selected from substituted or unsubstituted aryl with 6 to 25 carbon atoms, and substituted or unsubstituted heteroaryl with 5 to 20 carbon atoms. For example, Ar₁ is selected from substituted or unsubstituted aryl with 6, 7, 8, 9, 10, 11, 12, 13, 14, 15, 16, 17, 18, 19, 20, 21, 22, 23, 24, or 25 carbon atoms, and substituted or unsubstituted heteroaryl with 5, 6, 7, 8, 9, 10, 11, 12, 13, 14, 15, 16, 17, 18, 19, or 20 carbon atoms.

Optionally, a substituent in Ar₁ is selected from deuterium, cyano, fluorine, alkyl with 1 to 5 carbon atoms, trimethylsilyl, aryl with 6 to 12 carbon atoms, and heteroaryl with 5 to 12 carbon atoms.

Optionally, Ar₁ is selected from substituted or unsubstituted phenyl, substituted or unsubstituted naphthyl, substituted or unsubstituted biphenyl, substituted or unsubstituted diphenylfuranyl, substituted or unsubstituted dibenzothienyl, substituted or unsubstituted fluorenyl, substituted or unsubstituted carbazolyl, substituted or unsubstituted spirofluorenyl, and substituted or unsubstituted phenanthryl.

Preferably, substituents in Ar₁ are each independently selected from deuterium, cyano, fluorine, methyl, ethyl, isopropyl, tert-butyl, phenyl, naphthyl, biphenyl, pyridyl, and trimethylsilyl.

In the present disclosure, carbazolyl includes

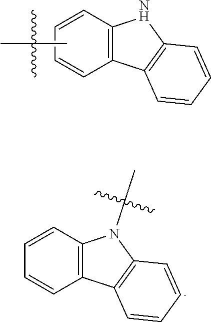

Optionally, Ar₁ is selected from a substituted or unsubstituted group Q, and the unsubstituted group Q is selected from the group consisting of:

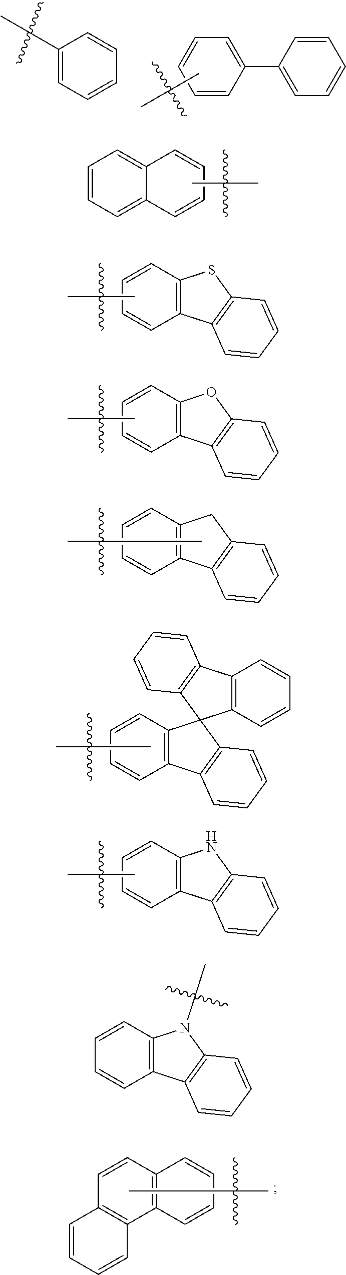

and

the substituted group Q has one or two or more substituents, and the substituents are each independently selected from deuterium, cyano, fluorine, methyl, ethyl, isopropyl, tert-butyl, phenyl, naphthyl, biphenyl, pyridyl, and trimethylsilyl; and when the number of the substituents is greater than 1, the substituents are the same or different.

Optionally, Ar is selected from the group consisting of:

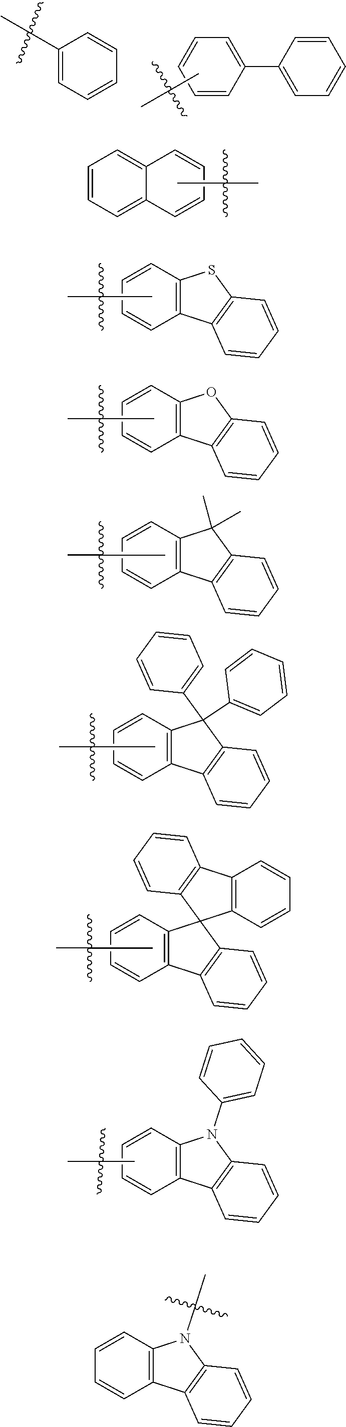

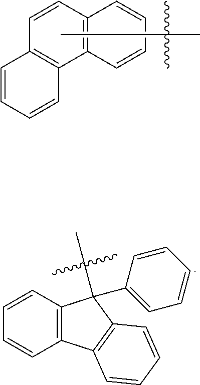

Further optionally, Ar is selected from the group consisting of:

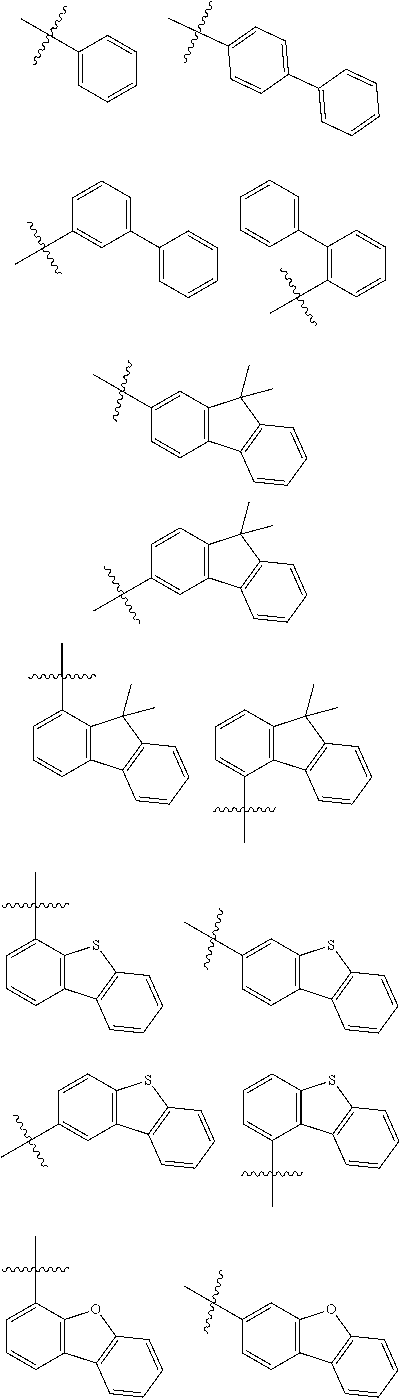

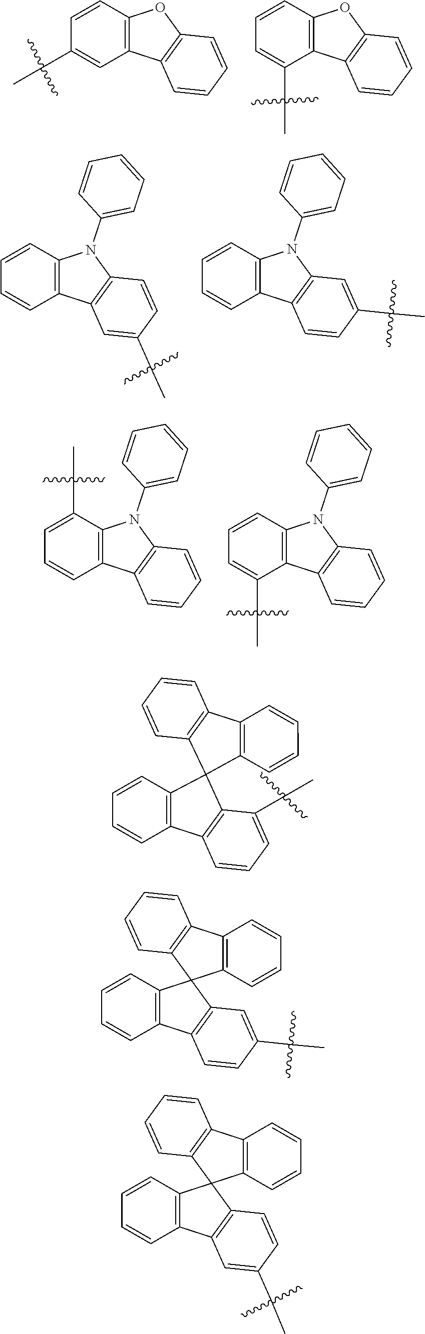

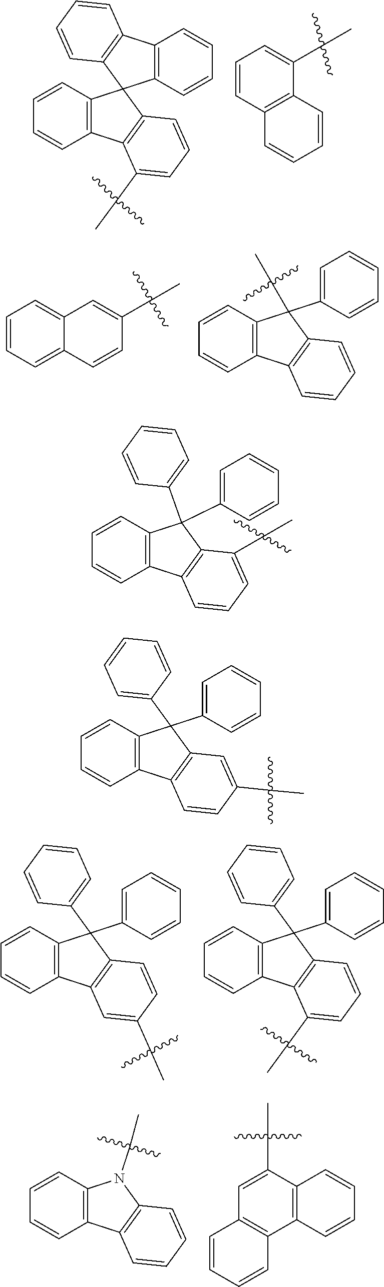

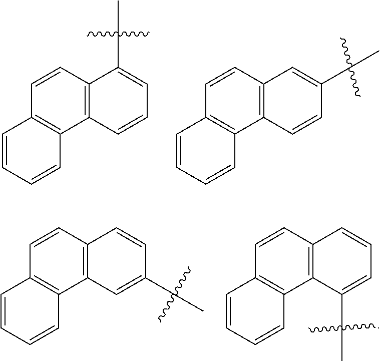

In one specific example of the present disclosure, L₁ and L₂ are each independently selected from a single bond, substituted or unsubstituted arylene with 6 to 20 carbon atoms, and substituted or unsubstituted heteroarylene with 10 to 20 carbon atoms. For example, L₁ and L₂ are each independently selected from a single bond, substituted or unsubstituted arylene with 6, 7, 8, 9, 10, 11, 12, 13, 14, 15, 16, 17, 18, 19, or 20 carbon atoms, and substituted or unsubstituted heteroarylene with 10, 11, 12, 13, 14, 15, 16, 17, 18, 19, or 20 carbon atoms.

Preferably, substituents in L₁ and L₂ are each independently selected from deuterium, cyano, fluorine, methyl, ethyl, isopropyl, tert-butyl, phenyl, naphthyl, biphenyl, pyridyl and trimethylsilyl.

Optionally, L₁ and L₂ are each independently selected from a single bond, substituted or unsubstituted phenylene, substituted or unsubstituted biphenylene, substituted or unsubstituted naphthylene, substituted or unsubstituted carbazolylene, substituted or unsubstituted dibenzothenylene, substituted or unsubstituted dibenzothenylene, and substituted or unsubstituted fluorenylene.

Preferably, the substituents in L₁ and L₂ are each independently selected from deuterium, cyano, fluorine, trimethylsilyl, methyl, ethyl, isopropyl, tert-butyl, phenyl, naphthyl, biphenyl and pyridyl.

Optionally, L₁ and L₂ are each independently selected from a single bond, and a substituted or unsubstituted group V, and the unsubstituted group V is selected from the group consisting of:

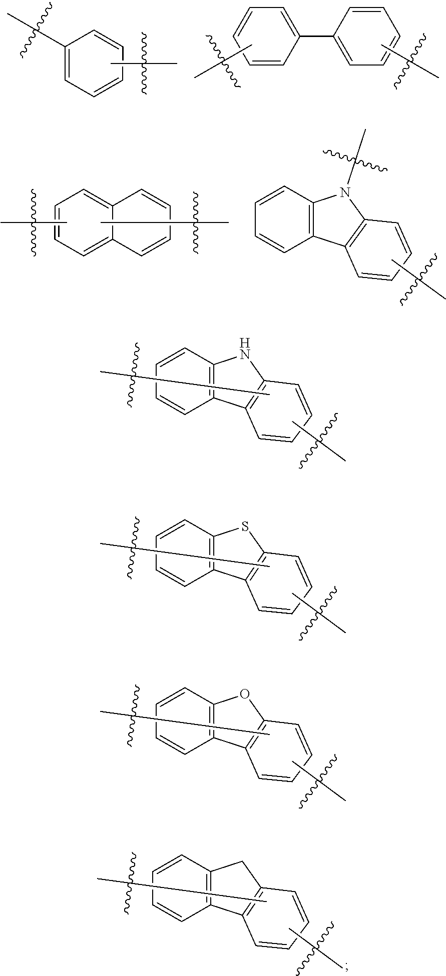

and

the substituted group V has one or two or more substituents, and the substituents are each independently selected from deuterium, cyano, fluorine, methyl, ethyl, isopropyl, tert-butyl, phenyl, naphthyl, biphenyl, pyridyl and trimethylsilyl; and when the number of the substituents is greater than 1, the substituents are the same or different.

Optionally, L₁ and L₂ are each independently selected from a single bond, and the group consisting of:

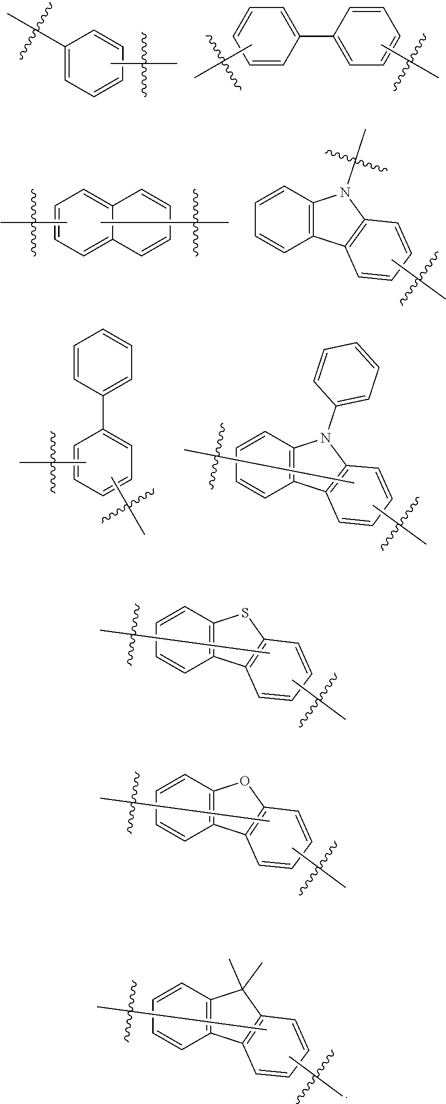

Further optionally, L₁ and L₂ are each independently selected from a single bond, and the group consisting of:

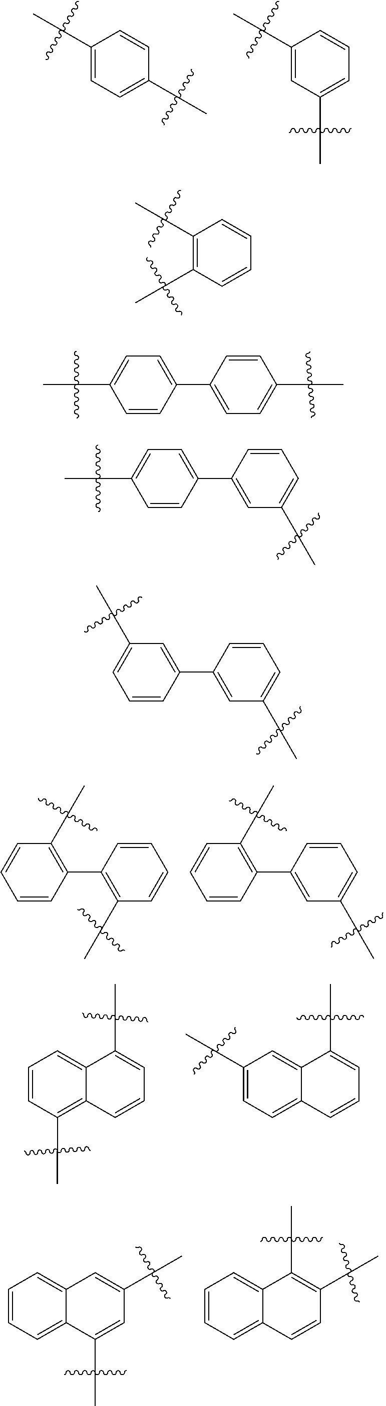

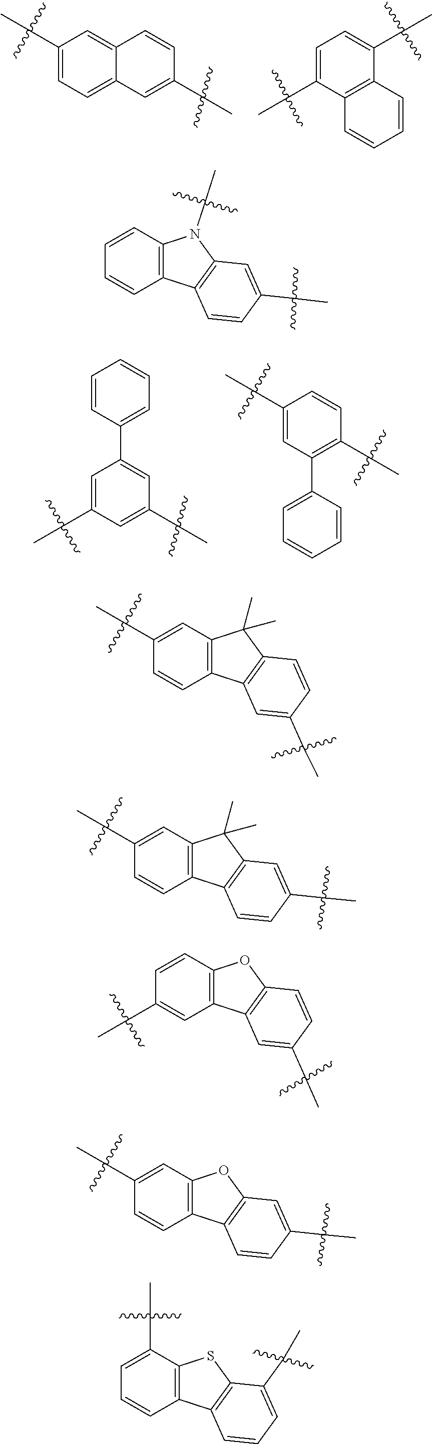

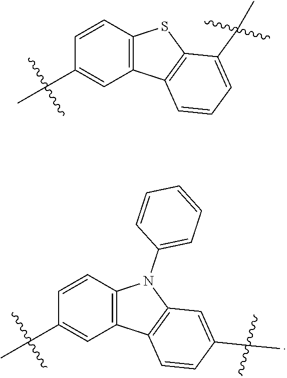 [SMILES: *c1ccc2sc3c(*)cccc3c2c1 ; *c1ccc2c(c1)c1ccc(*)cc1n2-c1ccccc1]

In one specific example of the present disclosure, R₁, R₂ and R₃ are each independently selected from deuterium, cyano, fluorine, methyl, ethyl, isopropyl, tert-butyl, phenyl, naphthyl, biphenyl, pyridyl, trimethylsilyl, dibenzofuranyl and dibenzothienyl.

Optionally, R₄, R₅, R₆, R₇ and R₈ are each independently selected from hydrogen, methyl, ethyl, isopropyl, tert-butyl, phenyl, naphthyl, biphenyl, pyrimidinyl and pyridyl; or, R₄ and R₅ form a fluorene ring

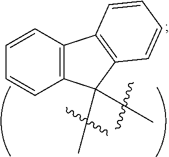

or, R₇ and R₈ form a fluorene ring

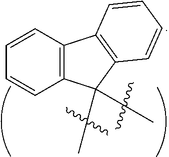

Preferably, R₄, R₅, R₆, R₇ and R₈ are each independently selected from methyl, ethyl, isopropyl, tert-butyl, phenyl, naphthyl, biphenyl, pyrimidinyl, and pyridyl; or, R₄ and R₅ form a fluorene ring; or, R₇ and R₈ form a fluorene ring.

Optionally, Ar₂ is selected from substituted or unsubstituted dibenzofuranyl, substituted or unsubstituted dibenzothienyl, substituted or unsubstituted fluorenyl, substituted or unsubstituted carbazolyl, substituted or unsubstituted silafluorenyl and substituted or unsubstituted spirofluorenyl.

Preferably, substituents in Ar₂ are each independently selected from deuterium, cyano, fluorine, trimethylsilyl, methyl, ethyl, isopropyl, tert-butyl, phenyl, naphthyl, biphenyl and pyridyl.

Optionally, Ar₂ is selected from a substituted or unsubstituted group W, and the unsubstituted group W is selected from the group consisting of:

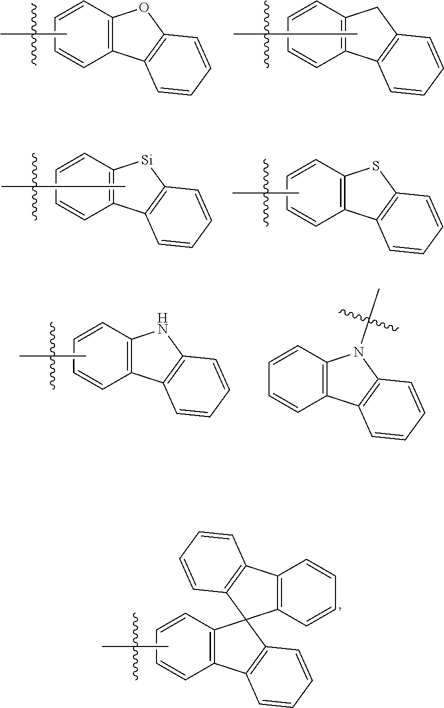

and

the substituted group W has one or two or more substituents, and the substituents are each independently selected from deuterium, cyano, fluorine, methyl, ethyl, isopropyl, tert-butyl, phenyl, naphthyl, biphenyl, pyridyl and trimethylsilyl; and when the number of the substituents is greater than 1, the substituents are the same or different.

Optionally, Ar₂ is selected from the group consisting of:

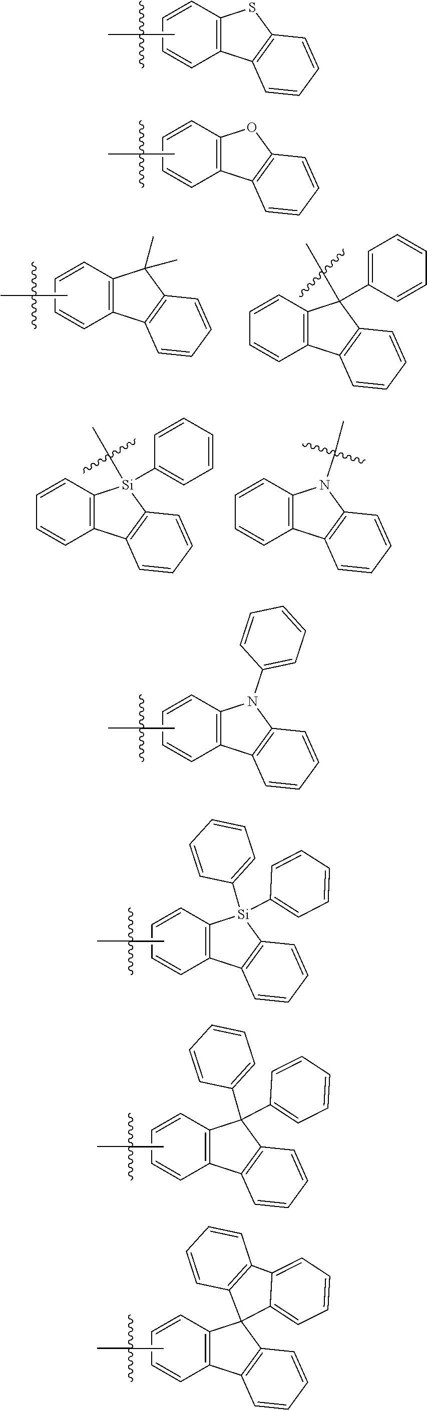

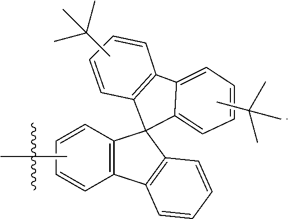

Further optionally, Ar₂ is selected from the group consisting of:

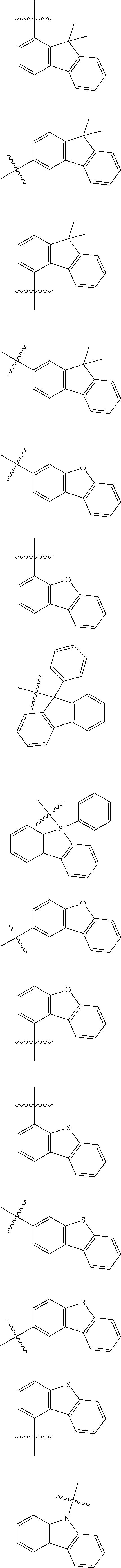

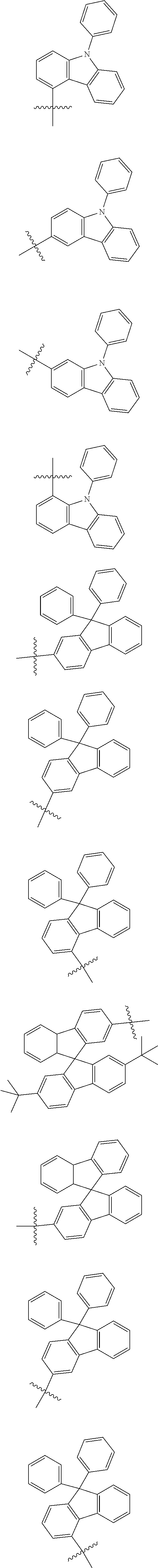

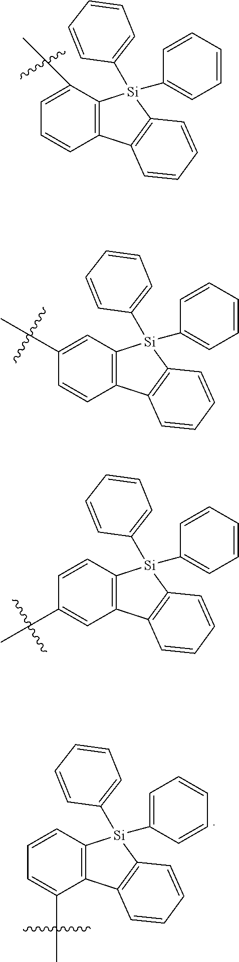

Optionally, A is selected from the group consisting of:

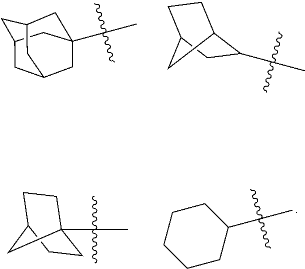 [SMILES: *C12CC3CC(CC(C3)C1)C2 ; *C1CC2CCC1C2 ; *C12CCC(CC1)C2 ; *C1CCCCC1]

Optionally, the organic compound is selected from the following organic compounds:

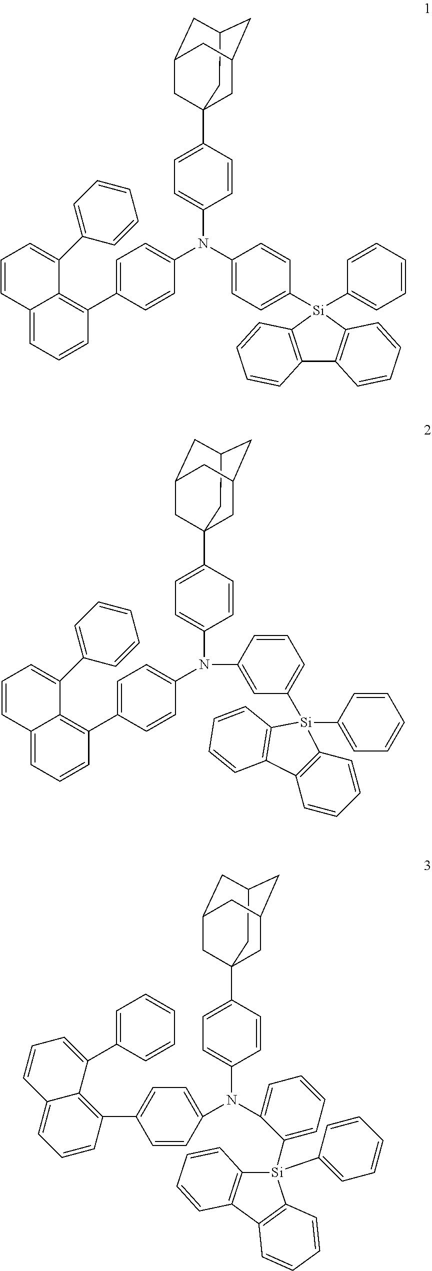 [SMILES: c1ccc(-c2cccc3cccc(-c4ccc(N(c5ccc(C67CC8CC(CC(C8)C6)C7)cc5)c5ccc([Si]6(c7ccccc7)c7ccccc7-c7ccccc76)cc5)cc4)c23)cc1 ; c1ccc(-c2cccc3cccc(-c4ccc(N(c5ccc(C67CC8CC(CC(C8)C6)C7)cc5)c5cccc([Si]6(c7ccccc7)c7ccccc7-c7ccccc76)c5)cc4)c23)cc1 ; c1ccc(-c2cccc3cccc(-c4ccc(N(c5ccc(C67CC8CC(CC(C8)C6)C7)cc5)c5ccccc5[Si]5(c6ccccc6)c6ccccc6-c6ccccc65)cc4)c23)cc1]

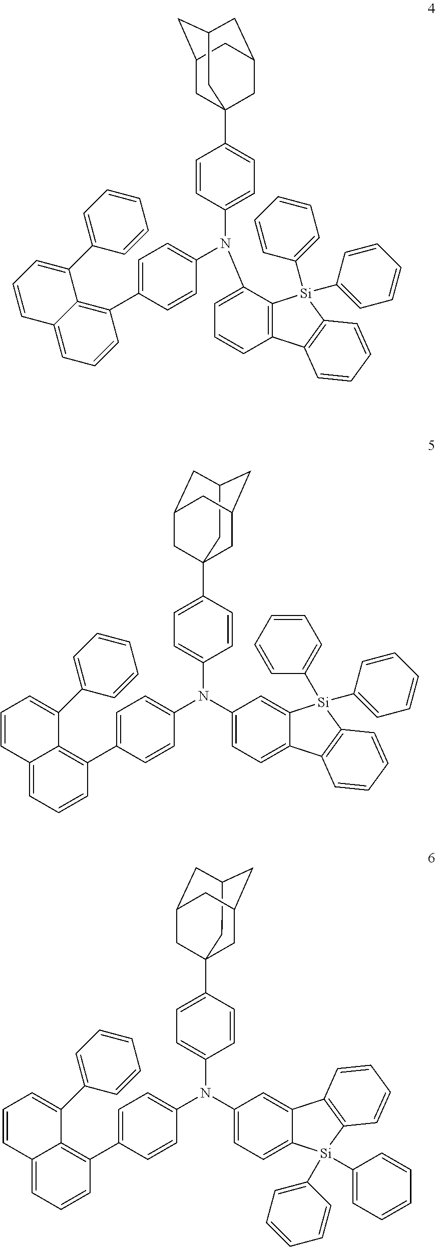 [SMILES: c1ccc(-c2cccc3cccc(-c4ccc(N(c5ccc(C67CC8CC(CC(C8)C6)C7)cc5)c5cccc6c5[Si](c5ccccc5)(c5ccccc5)c5ccccc5-6)cc4)c23)cc1 ; c1ccc(-c2cccc3cccc(-c4ccc(N(c5ccc(C67CC8CC(CC(C8)C6)C7)cc5)c5ccc6c(c5)[Si](c5ccccc5)(c5ccccc5)c5ccccc5-6)cc4)c23)cc1 ; c1ccc(-c2cccc3cccc(-c4ccc(N(c5ccc(C67CC8CC(CC(C8)C6)C7)cc5)c5ccc6c(c5)-c5ccccc5[Si]6(c5ccccc5)c5ccccc5)cc4)c23)cc1]

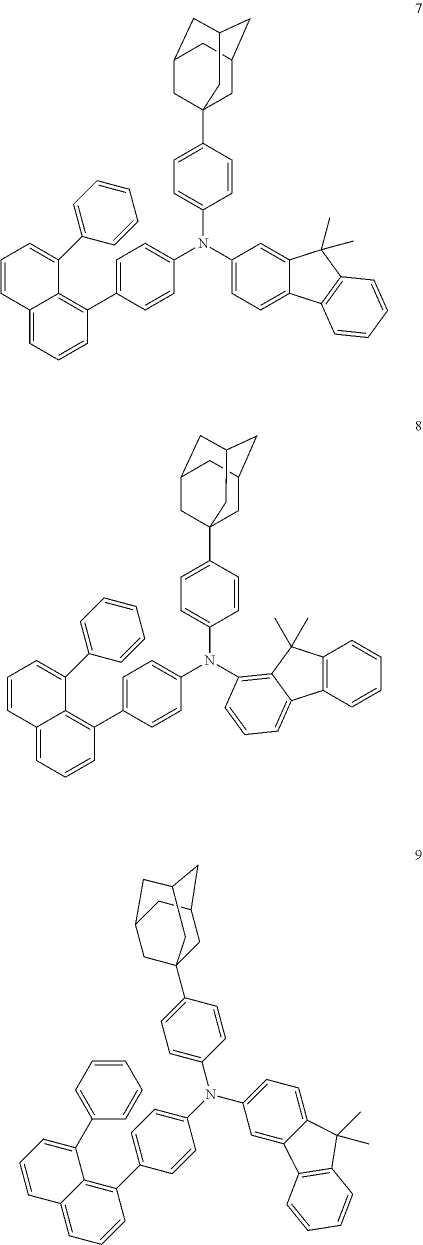 [SMILES: CC1(C)c2ccccc2-c2ccc(N(c3ccc(-c4cccc5cccc(-c6ccccc6)c45)cc3)c3ccc(C45CC6CC(CC(C6)C4)C5)cc3)cc21 ; CC1(C)c2ccccc2-c2cccc(N(c3ccc(-c4cccc5cccc(-c6ccccc6)c45)cc3)c3ccc(C45CC6CC(CC(C6)C4)C5)cc3)c21 ; CC1(C)c2ccccc2-c2cc(N(c3ccc(-c4cccc5cccc(-c6ccccc6)c45)cc3)c3ccc(C45CC6CC(CC(C6)C4)C5)cc3)ccc21]

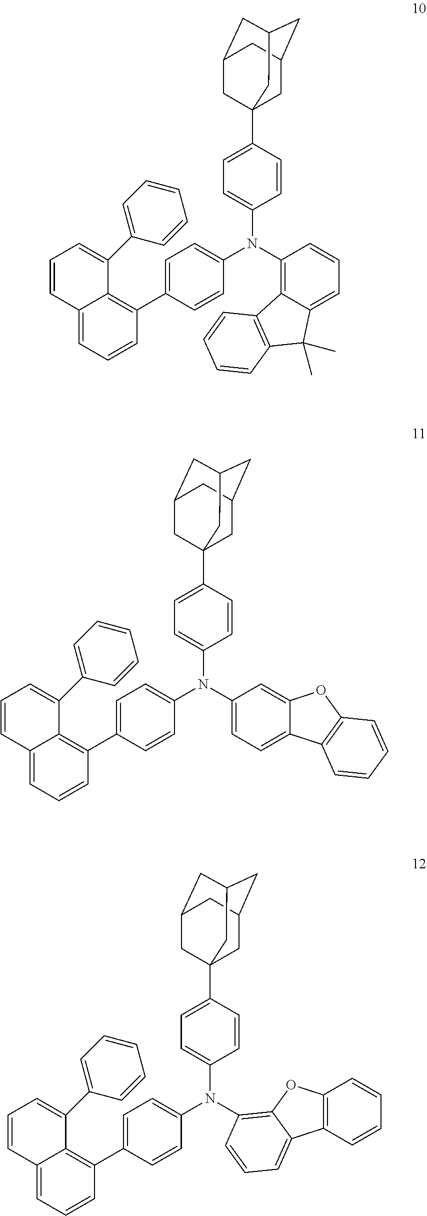 [SMILES: CC1(C)c2ccccc2-c2c(N(c3ccc(-c4cccc5cccc(-c6ccccc6)c45)cc3)c3ccc(C45CC6CC(CC(C6)C4)C5)cc3)cccc21 ; c1ccc(-c2cccc3cccc(-c4ccc(N(c5ccc(C67CC8CC(CC(C8)C6)C7)cc5)c5ccc6c(c5)oc5ccccc56)cc4)c23)cc1 ; c1ccc(-c2cccc3cccc(-c4ccc(N(c5ccc(C67CC8CC(CC(C8)C6)C7)cc5)c5cccc6c5oc5ccccc56)cc4)c23)cc1]

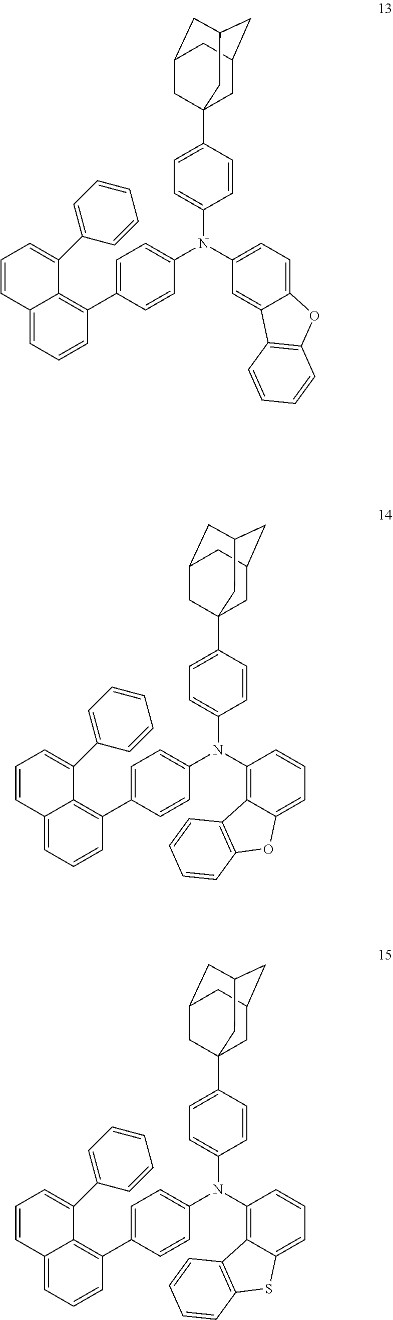 [SMILES: c1ccc(-c2cccc3cccc(-c4ccc(N(c5ccc(C67CC8CC(CC(C8)C6)C7)cc5)c5ccc6oc7ccccc7c6c5)cc4)c23)cc1 ; c1ccc(-c2cccc3cccc(-c4ccc(N(c5ccc(C67CC8CC(CC(C8)C6)C7)cc5)c5cccc6oc7ccccc7c56)cc4)c23)cc1 ; c1ccc(-c2cccc3cccc(-c4ccc(N(c5ccc(C67CC8CC(CC(C8)C6)C7)cc5)c5cccc6sc7ccccc7c56)cc4)c23)cc1]

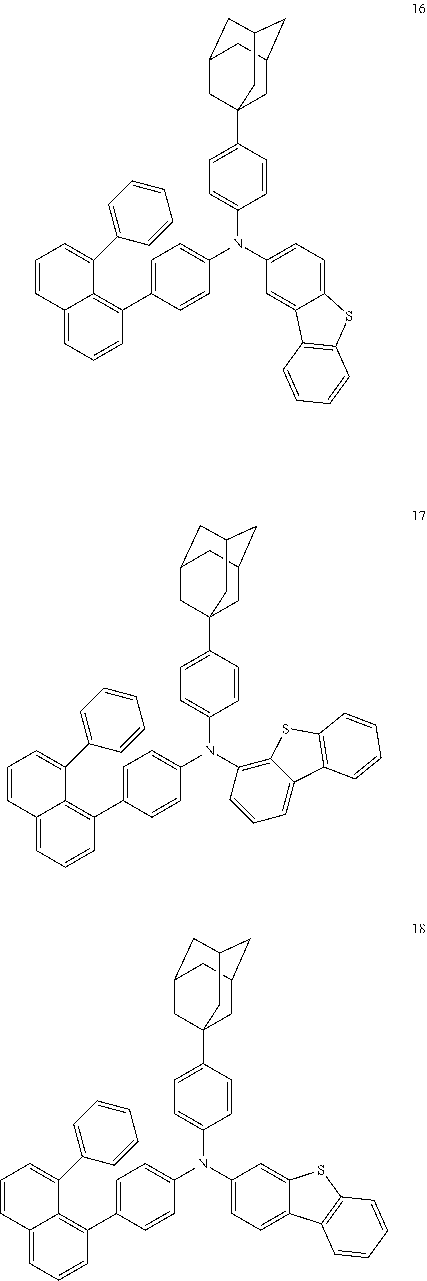 [SMILES: c1ccc(-c2cccc3cccc(-c4ccc(N(c5ccc(C67CC8CC(CC(C8)C6)C7)cc5)c5ccc6sc7ccccc7c6c5)cc4)c23)cc1 ; c1ccc(-c2cccc3cccc(-c4ccc(N(c5ccc(C67CC8CC(CC(C8)C6)C7)cc5)c5cccc6c5sc5ccccc56)cc4)c23)cc1 ; c1ccc(-c2cccc3cccc(-c4ccc(N(c5ccc(C67CC8CC(CC(C8)C6)C7)cc5)c5ccc6c(c5)sc5ccccc56)cc4)c23)cc1]

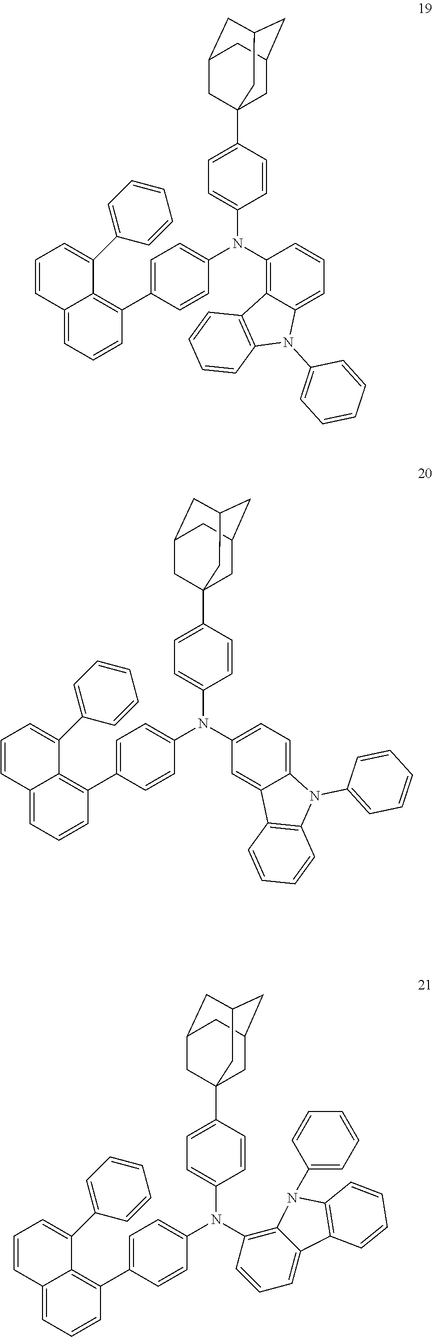 [SMILES: c1ccc(-c2cccc3cccc(-c4ccc(N(c5ccc(C67CC8CC(CC(C8)C6)C7)cc5)c5cccc6c5c5ccccc5n6-c5ccccc5)cc4)c23)cc1 ; c1ccc(-c2cccc3cccc(-c4ccc(N(c5ccc(C67CC8CC(CC(C8)C6)C7)cc5)c5ccc6c(c5)c5ccccc5n6-c5ccccc5)cc4)c23)cc1 ; c1ccc(-c2cccc3cccc(-c4ccc(N(c5ccc(C67CC8CC(CC(C8)C6)C7)cc5)c5cccc6c7ccccc7n(-c7ccccc7)c56)cc4)c23)cc1]

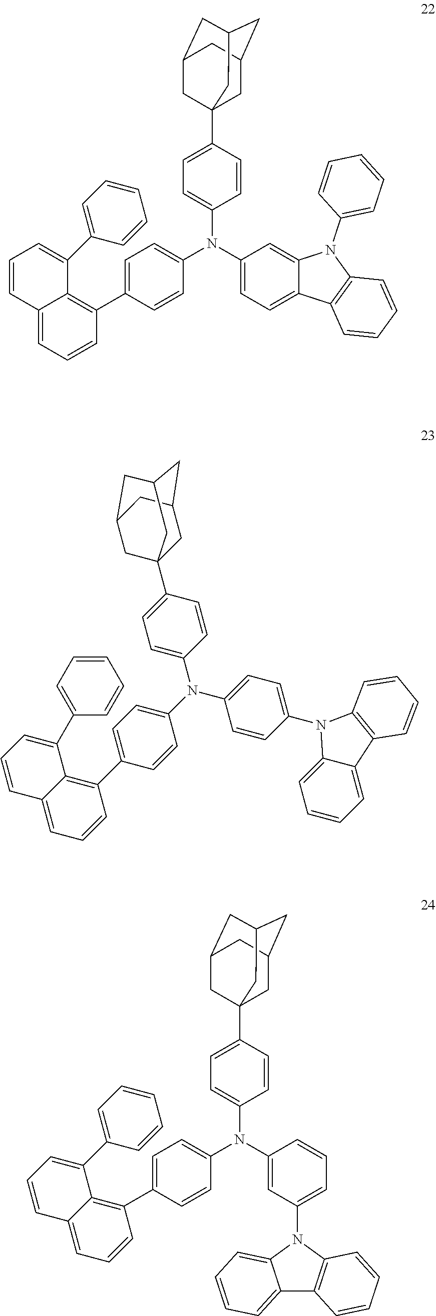 [SMILES: c1ccc(-c2cccc3cccc(-c4ccc(N(c5ccc(C67CC8CC(CC(C8)C6)C7)cc5)c5ccc6c7ccccc7n(-c7ccccc7)c6c5)cc4)c23)cc1 ; c1ccc(-c2cccc3cccc(-c4ccc(N(c5ccc(-n6c7ccccc7c7ccccc76)cc5)c5ccc(C67CC8CC(CC(C8)C6)C7)cc5)cc4)c23)cc1 ; c1ccc(-c2cccc3cccc(-c4ccc(N(c5ccc(C67CC8CC(CC(C8)C6)C7)cc5)c5cccc(-n6c7ccccc7c7ccccc76)c5)cc4)c23)cc1]

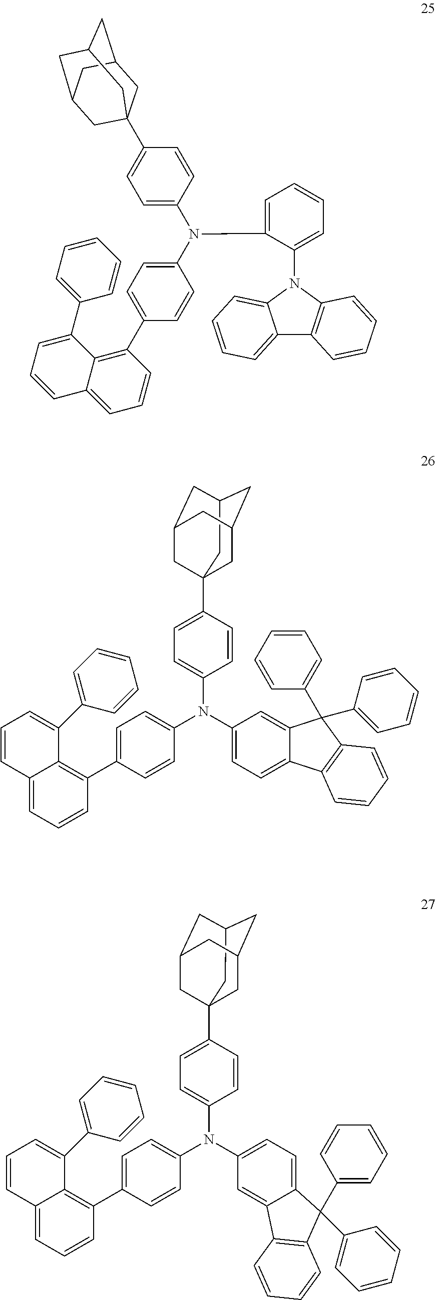 [SMILES: c1ccc(-c2cccc3cccc(-c4ccc(N(c5ccc(C67CC8CC(CC(C8)C6)C7)cc5)c5ccccc5-n5c6ccccc6c6ccccc65)cc4)c23)cc1 ; c1ccc(-c2cccc3cccc(-c4ccc(N(c5ccc(C67CC8CC(CC(C8)C6)C7)cc5)c5ccc6c(c5)C(c5ccccc5)(c5ccccc5)c5ccccc5-6)cc4)c23)cc1 ; c1ccc(-c2cccc3cccc(-c4ccc(N(c5ccc(C67CC8CC(CC(C8)C6)C7)cc5)c5ccc6c(c5)-c5ccccc5C6(c5ccccc5)c5ccccc5)cc4)c23)cc1]

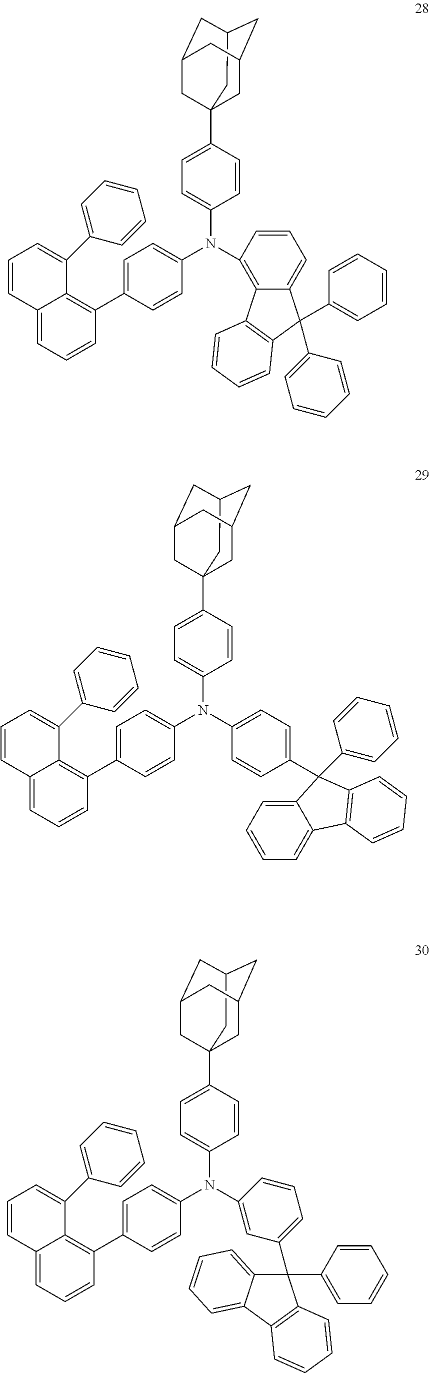 [SMILES: c1ccc(-c2cccc3cccc(-c4ccc(N(c5ccc(C67CC8CC(CC(C8)C6)C7)cc5)c5cccc6c5-c5ccccc5C6(c5ccccc5)c5ccccc5)cc4)c23)cc1 ; c1ccc(-c2cccc3cccc(-c4ccc(N(c5ccc(C67CC8CC(CC(C8)C6)C7)cc5)c5ccc(C6(c7ccccc7)c7ccccc7-c7ccccc76)cc5)cc4)c23)cc1 ; c1ccc(-c2cccc3cccc(-c4ccc(N(c5ccc(C67CC8CC(CC(C8)C6)C7)cc5)c5cccc(C6(c7ccccc7)c7ccccc7-c7ccccc76)c5)cc4)c23)cc1]

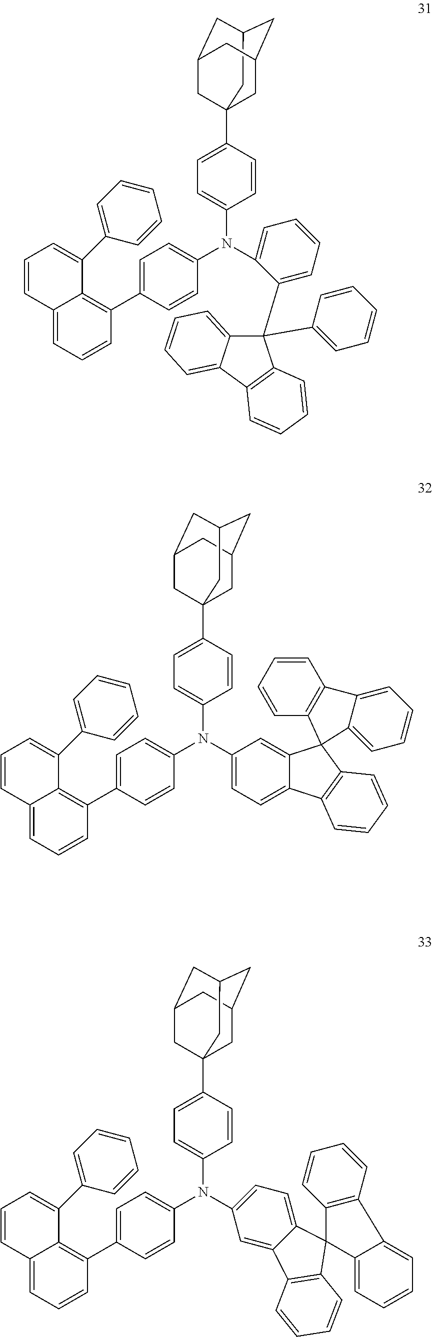 [SMILES: c1ccc(-c2cccc3cccc(-c4ccc(N(c5ccc(C67CC8CC(CC(C8)C6)C7)cc5)c5ccccc5C5(c6ccccc6)c6ccccc6-c6ccccc65)cc4)c23)cc1 ; c1ccc(-c2cccc3cccc(-c4ccc(N(c5ccc(C67CC8CC(CC(C8)C6)C7)cc5)c5ccc6c(c5)C5(c7ccccc7-c7ccccc75)c5ccccc5-6)cc4)c23)cc1 ; c1ccc(-c2cccc3cccc(-c4ccc(N(c5ccc(C67CC8CC(CC(C8)C6)C7)cc5)c5ccc6c(c5)-c5ccccc5C65c6ccccc6-c6ccccc65)cc4)c23)cc1]

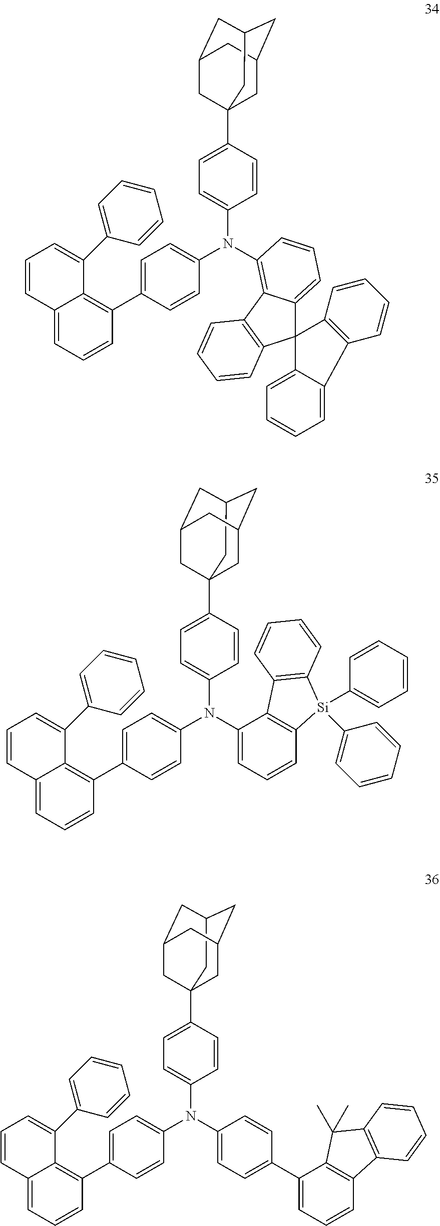 [SMILES: c1ccc(-c2cccc3cccc(-c4ccc(N(c5ccc(C67CC8CC(CC(C8)C6)C7)cc5)c5cccc6c5-c5ccccc5C65c6ccccc6-c6ccccc65)cc4)c23)cc1 ; c1ccc(-c2cccc3cccc(-c4ccc(N(c5ccc(C67CC8CC(CC(C8)C6)C7)cc5)c5cccc6c5-c5ccccc5[Si]6(c5ccccc5)c5ccccc5)cc4)c23)cc1 ; CC1(C)c2ccccc2-c2cccc(-c3ccc(N(c4ccc(-c5cccc6cccc(-c7ccccc7)c56)cc4)c4ccc(C56CC7CC(CC(C7)C5)C6)cc4)cc3)c21]

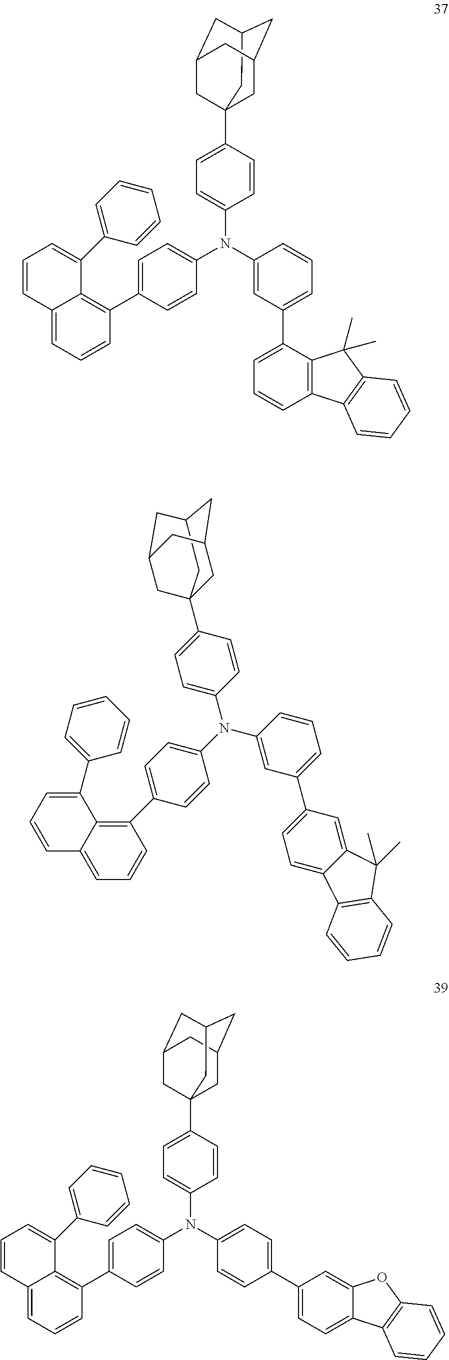 [SMILES: CC1(C)c2ccccc2-c2cccc(-c3cccc(N(c4ccc(-c5cccc6cccc(-c7ccccc7)c56)cc4)c4ccc(C56CC7CC(CC(C7)C5)C6)cc4)c3)c21 ; CC1(C)c2ccccc2-c2ccc(-c3cccc(N(c4ccc(-c5cccc6cccc(-c7ccccc7)c56)cc4)c4ccc(C56CC7CC(CC(C7)C5)C6)cc4)c3)cc21 ; c1ccc(-c2cccc3cccc(-c4ccc(N(c5ccc(-c6ccc7c(c6)oc6ccccc67)cc5)c5ccc(C67CC8CC(CC(C8)C6)C7)cc5)cc4)c23)cc1]

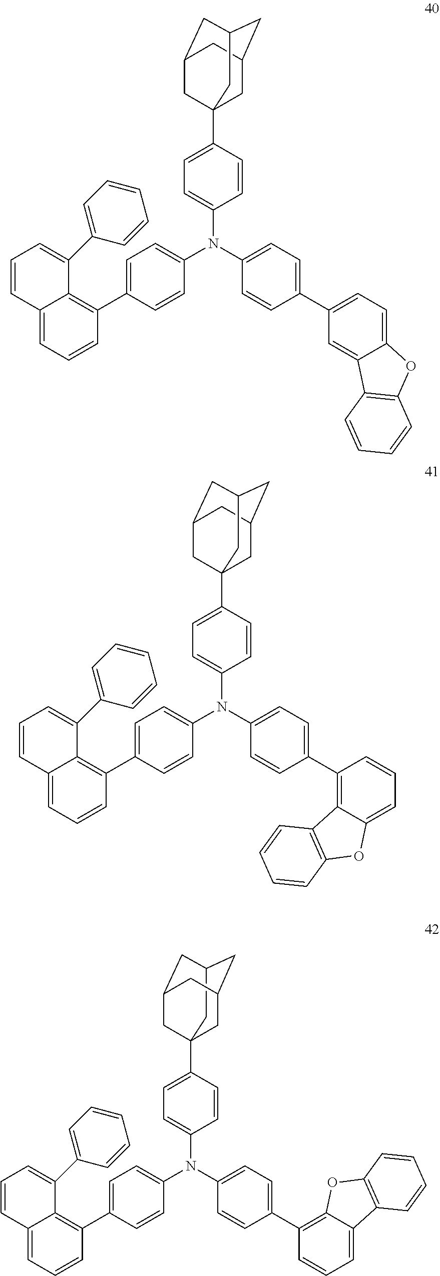 [SMILES: c1ccc(-c2cccc3cccc(-c4ccc(N(c5ccc(-c6ccc7oc8ccccc8c7c6)cc5)c5ccc(C67CC8CC(CC(C8)C6)C7)cc5)cc4)c23)cc1 ; c1ccc(-c2cccc3cccc(-c4ccc(N(c5ccc(-c6cccc7oc8ccccc8c67)cc5)c5ccc(C67CC8CC(CC(C8)C6)C7)cc5)cc4)c23)cc1 ; c1ccc(-c2cccc3cccc(-c4ccc(N(c5ccc(-c6cccc7c6oc6ccccc67)cc5)c5ccc(C67CC8CC(CC(C8)C6)C7)cc5)cc4)c23)cc1]

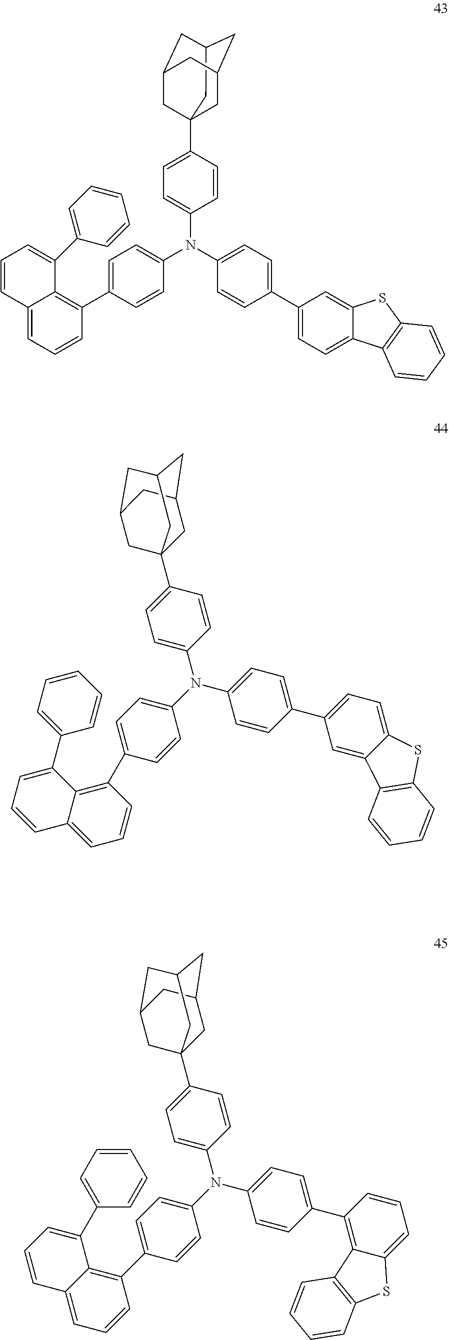 [SMILES: c1ccc(-c2cccc3cccc(-c4ccc(N(c5ccc(-c6ccc7c(c6)sc6ccccc67)cc5)c5ccc(C67CC8CC(CC(C8)C6)C7)cc5)cc4)c23)cc1 ; c1ccc(-c2cccc3cccc(-c4ccc(N(c5ccc(-c6ccc7sc8ccccc8c7c6)cc5)c5ccc(C67CC8CC(CC(C8)C6)C7)cc5)cc4)c23)cc1 ; c1ccc(-c2cccc3cccc(-c4ccc(N(c5ccc(-c6cccc7sc8ccccc8c67)cc5)c5ccc(C67CC8CC(CC(C8)C6)C7)cc5)cc4)c23)cc1]

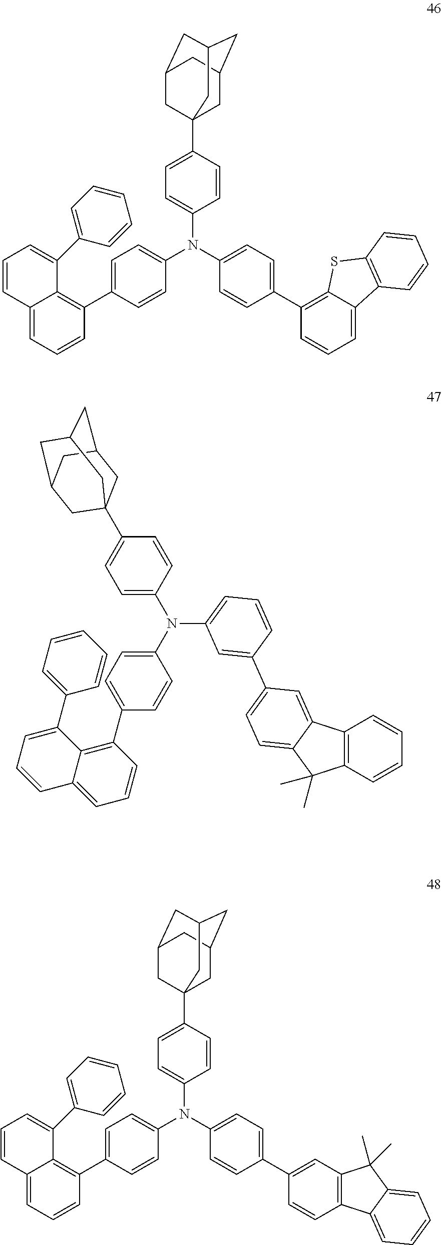 [SMILES: c1ccc(-c2cccc3cccc(-c4ccc(N(c5ccc(-c6cccc7c6sc6ccccc67)cc5)c5ccc(C67CC8CC(CC(C8)C6)C7)cc5)cc4)c23)cc1 ; CC1(C)c2ccccc2-c2cc(-c3cccc(N(c4ccc(-c5cccc6cccc(-c7ccccc7)c56)cc4)c4ccc(C56CC7CC(CC(C7)C5)C6)cc4)c3)ccc21 ; CC1(C)c2ccccc2-c2ccc(-c3ccc(N(c4ccc(-c5cccc6cccc(-c7ccccc7)c56)cc4)c4ccc(C56CC7CC(CC(C7)C5)C6)cc4)cc3)cc21]

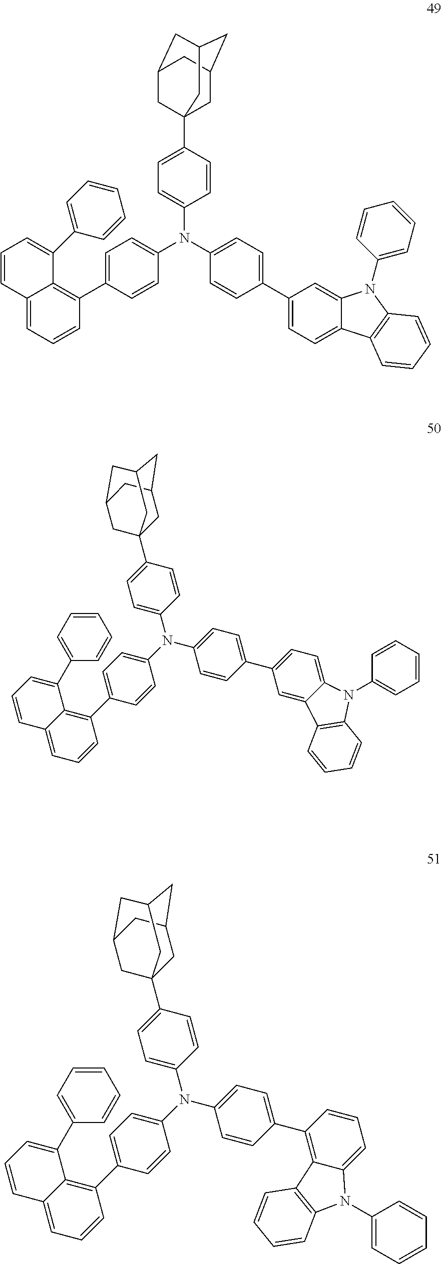 [SMILES: c1ccc(-c2cccc3cccc(-c4ccc(N(c5ccc(-c6ccc7c8ccccc8n(-c8ccccc8)c7c6)cc5)c5ccc(C67CC8CC(CC(C8)C6)C7)cc5)cc4)c23)cc1 ; c1ccc(-c2cccc3cccc(-c4ccc(N(c5ccc(-c6ccc7c(c6)c6ccccc6n7-c6ccccc6)cc5)c5ccc(C67CC8CC(CC(C8)C6)C7)cc5)cc4)c23)cc1 ; c1ccc(-c2cccc3cccc(-c4ccc(N(c5ccc(-c6cccc7c6c6ccccc6n7-c6ccccc6)cc5)c5ccc(C67CC8CC(CC(C8)C6)C7)cc5)cc4)c23)cc1]

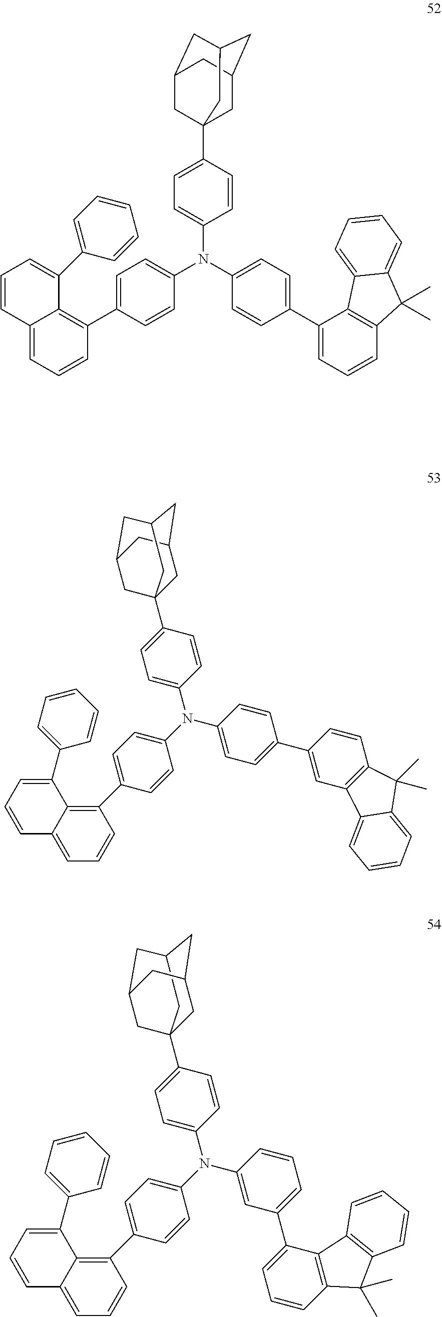 [SMILES: CC1(C)c2ccccc2-c2c(-c3ccc(N(c4ccc(-c5cccc6cccc(-c7ccccc7)c56)cc4)c4ccc(C56CC7CC(CC(C7)C5)C6)cc4)cc3)cccc21 ; CC1(C)c2ccccc2-c2cc(-c3ccc(N(c4ccc(-c5cccc6cccc(-c7ccccc7)c56)cc4)c4ccc(C56CC7CC(CC(C7)C5)C6)cc4)cc3)ccc21 ; CC1(C)c2ccccc2-c2c(-c3cccc(N(c4ccc(-c5cccc6cccc(-c7ccccc7)c56)cc4)c4ccc(C56CC7CC(CC(C7)C5)C6)cc4)c3)cccc21]

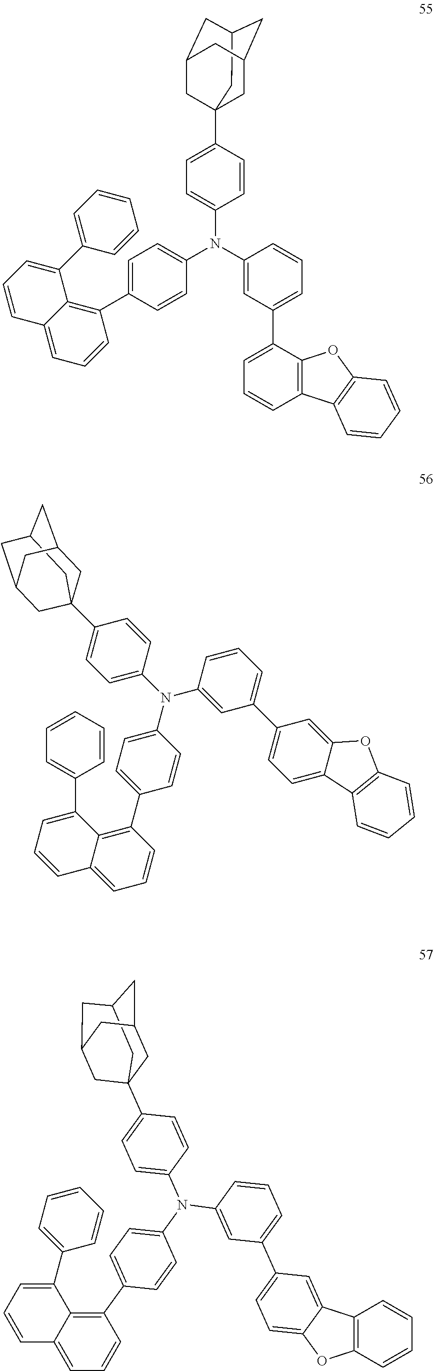 [SMILES: c1ccc(-c2cccc3cccc(-c4ccc(N(c5ccc(C67CC8CC(CC(C8)C6)C7)cc5)c5cccc(-c6cccc7c6oc6ccccc67)c5)cc4)c23)cc1 ; c1ccc(-c2cccc3cccc(-c4ccc(N(c5ccc(C67CC8CC(CC(C8)C6)C7)cc5)c5cccc(-c6ccc7c(c6)oc6ccccc67)c5)cc4)c23)cc1 ; c1ccc(-c2cccc3cccc(-c4ccc(N(c5ccc(C67CC8CC(CC(C8)C6)C7)cc5)c5cccc(-c6ccc7oc8ccccc8c7c6)c5)cc4)c23)cc1]

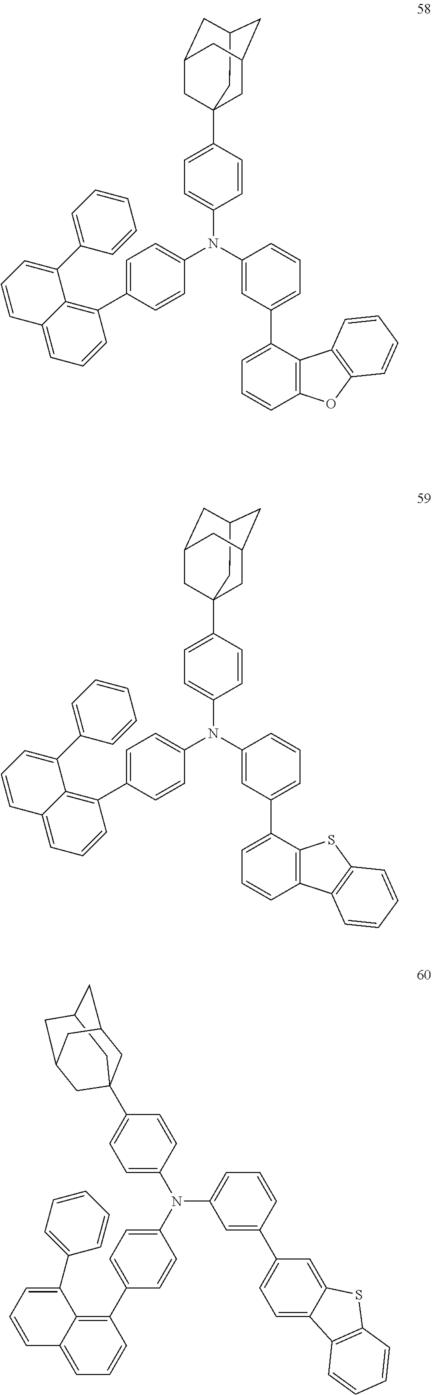 [SMILES: c1ccc(-c2cccc3cccc(-c4ccc(N(c5ccc(C67CC8CC(CC(C8)C6)C7)cc5)c5cccc(-c6cccc7oc8ccccc8c67)c5)cc4)c23)cc1 ; c1ccc(-c2cccc3cccc(-c4ccc(N(c5ccc(C67CC8CC(CC(C8)C6)C7)cc5)c5cccc(-c6cccc7c6sc6ccccc67)c5)cc4)c23)cc1 ; c1ccc(-c2cccc3cccc(-c4ccc(N(c5ccc(C67CC8CC(CC(C8)C6)C7)cc5)c5cccc(-c6ccc7c(c6)sc6ccccc67)c5)cc4)c23)cc1]

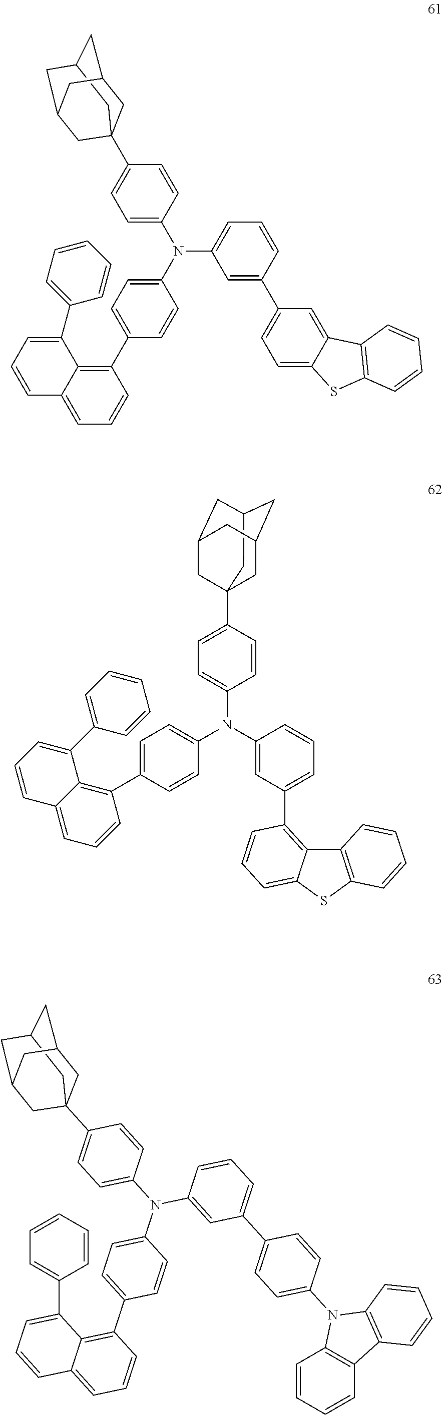 [SMILES: c1ccc(-c2cccc3cccc(-c4ccc(N(c5ccc(C67CC8CC(CC(C8)C6)C7)cc5)c5cccc(-c6ccc7sc8ccccc8c7c6)c5)cc4)c23)cc1 ; c1ccc(-c2cccc3cccc(-c4ccc(N(c5ccc(C67CC8CC(CC(C8)C6)C7)cc5)c5cccc(-c6cccc7sc8ccccc8c67)c5)cc4)c23)cc1 ; c1ccc(-c2cccc3cccc(-c4ccc(N(c5ccc(C67CC8CC(CC(C8)C6)C7)cc5)c5cccc(-c6ccc(-n7c8ccccc8c8ccccc87)cc6)c5)cc4)c23)cc1]

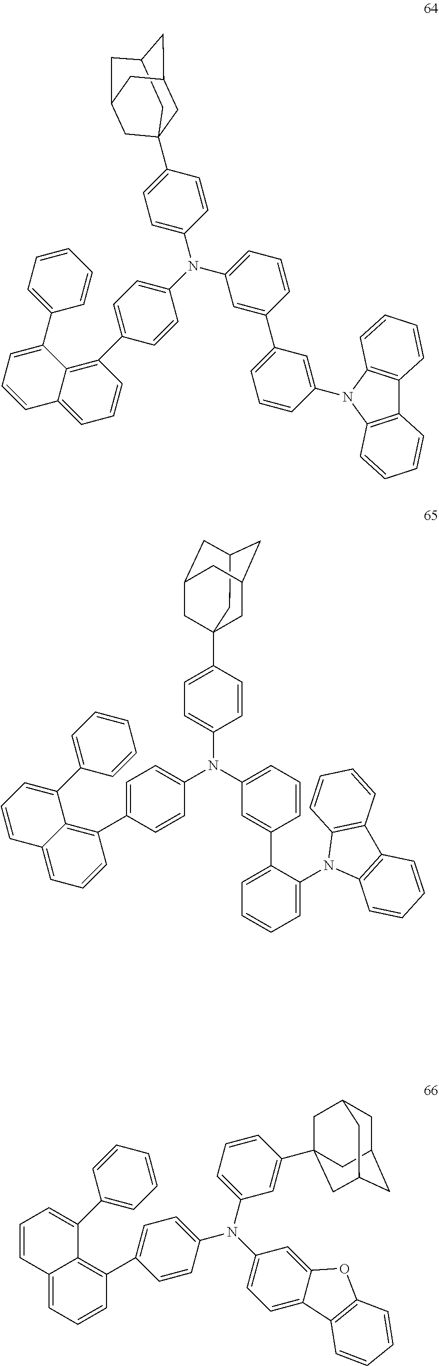 [SMILES: c1ccc(-c2cccc3cccc(-c4ccc(N(c5ccc(C67CC8CC(CC(C8)C6)C7)cc5)c5cccc(-c6cccc(-n7c8ccccc8c8ccccc87)c6)c5)cc4)c23)cc1 ; c1ccc(-c2cccc3cccc(-c4ccc(N(c5ccc(C67CC8CC(CC(C8)C6)C7)cc5)c5cccc(-c6ccccc6-n6c7ccccc7c7ccccc76)c5)cc4)c23)cc1 ; c1ccc(-c2cccc3cccc(-c4ccc(N(c5cccc(C67CC8CC(CC(C8)C6)C7)c5)c5ccc6c(c5)oc5ccccc56)cc4)c23)cc1]

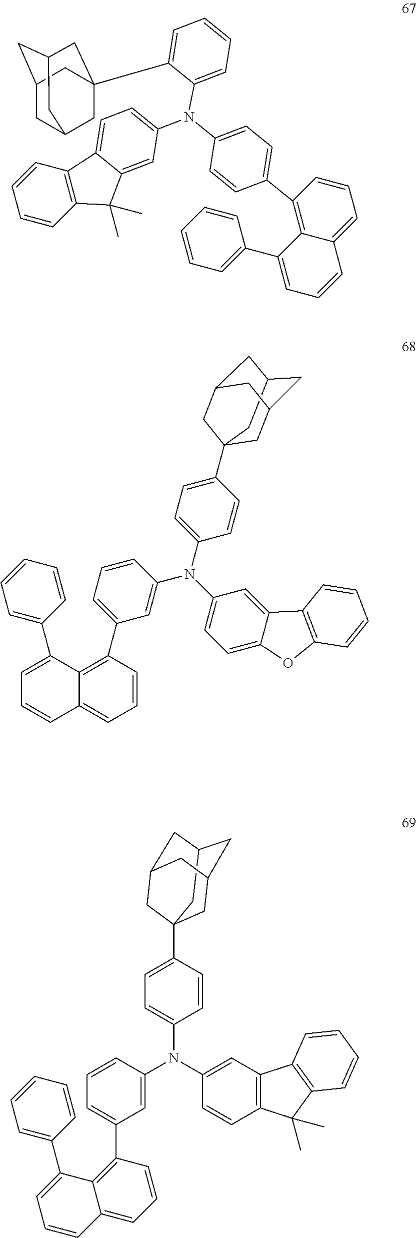 [SMILES: CC1(C)c2ccccc2-c2ccc(N(c3ccc(-c4cccc5cccc(-c6ccccc6)c45)cc3)c3ccccc3C34CC5CC(CC(C5)C3)C4)cc21 ; c1ccc(-c2cccc3cccc(-c4cccc(N(c5ccc(C67CC8CC(CC(C8)C6)C7)cc5)c5ccc6oc7ccccc7c6c5)c4)c23)cc1 ; CC1(C)c2ccccc2-c2cc(N(c3ccc(C45CC6CC(CC(C6)C4)C5)cc3)c3cccc(-c4cccc5cccc(-c6ccccc6)c45)c3)ccc21]

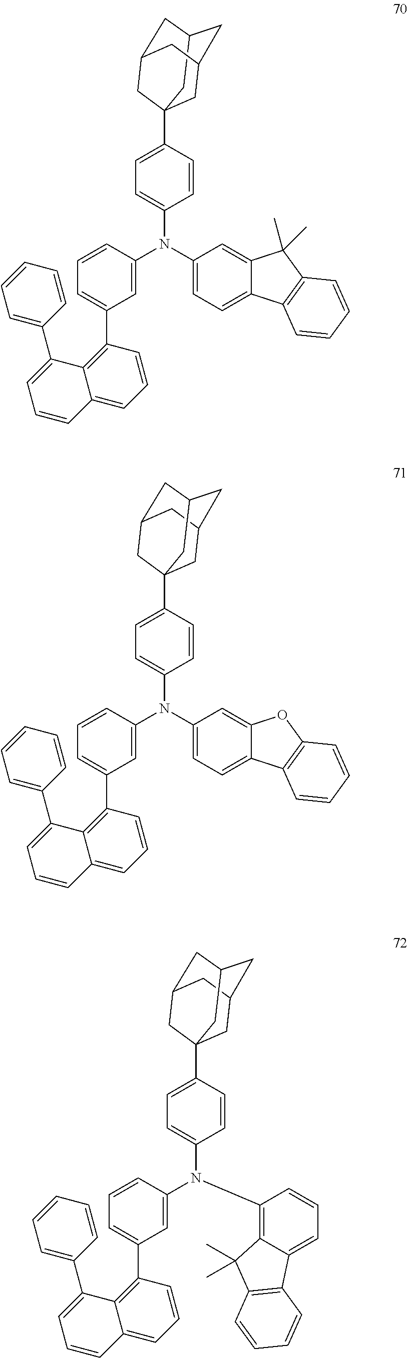 [SMILES: CC1(C)c2ccccc2-c2ccc(N(c3ccc(C45CC6CC(CC(C6)C4)C5)cc3)c3cccc(-c4cccc5cccc(-c6ccccc6)c45)c3)cc21 ; c1ccc(-c2cccc3cccc(-c4cccc(N(c5ccc(C67CC8CC(CC(C8)C6)C7)cc5)c5ccc6c(c5)oc5ccccc56)c4)c23)cc1 ; CC1(C)c2ccccc2-c2cccc(N(c3ccc(C45CC6CC(CC(C6)C4)C5)cc3)c3cccc(-c4cccc5cccc(-c6ccccc6)c45)c3)c21]

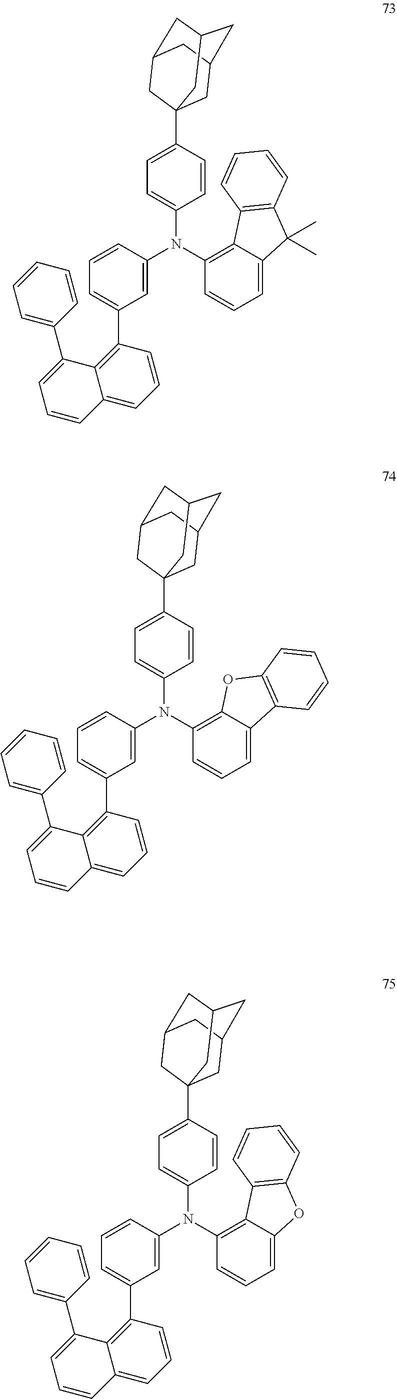 [SMILES: CC1(C)c2ccccc2-c2c(N(c3ccc(C45CC6CC(CC(C6)C4)C5)cc3)c3cccc(-c4cccc5cccc(-c6ccccc6)c45)c3)cccc21 ; c1ccc(-c2cccc3cccc(-c4cccc(N(c5ccc(C67CC8CC(CC(C8)C6)C7)cc5)c5cccc6c5oc5ccccc56)c4)c23)cc1 ; c1ccc(-c2cccc3cccc(-c4cccc(N(c5ccc(C67CC8CC(CC(C8)C6)C7)cc5)c5cccc6oc7ccccc7c56)c4)c23)cc1]

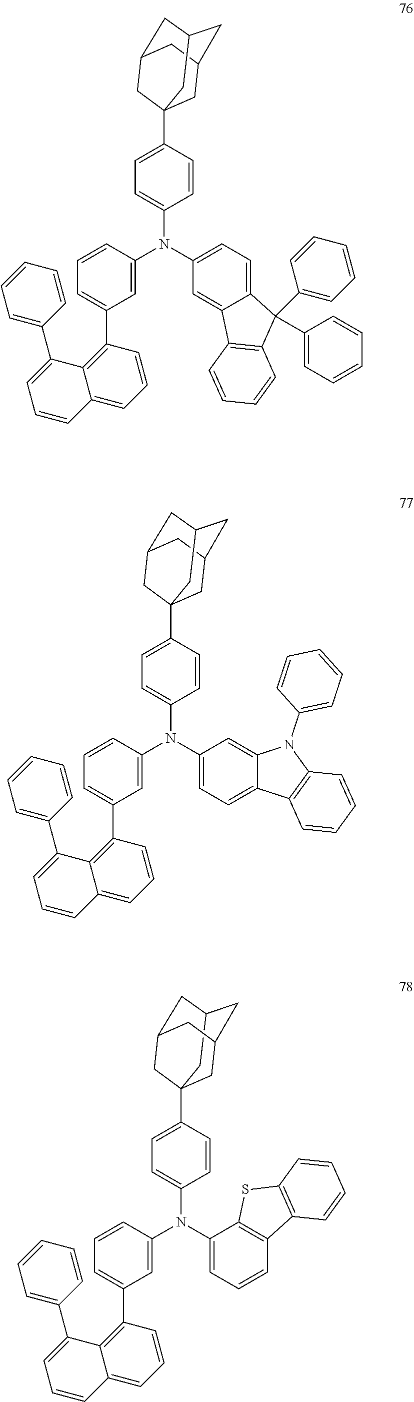 [SMILES: c1ccc(-c2cccc3cccc(-c4cccc(N(c5ccc(C67CC8CC(CC(C8)C6)C7)cc5)c5ccc6c(c5)-c5ccccc5C6(c5ccccc5)c5ccccc5)c4)c23)cc1 ; c1ccc(-c2cccc3cccc(-c4cccc(N(c5ccc(C67CC8CC(CC(C8)C6)C7)cc5)c5ccc6c7ccccc7n(-c7ccccc7)c6c5)c4)c23)cc1 ; c1ccc(-c2cccc3cccc(-c4cccc(N(c5ccc(C67CC8CC(CC(C8)C6)C7)cc5)c5cccc6c5sc5ccccc56)c4)c23)cc1]

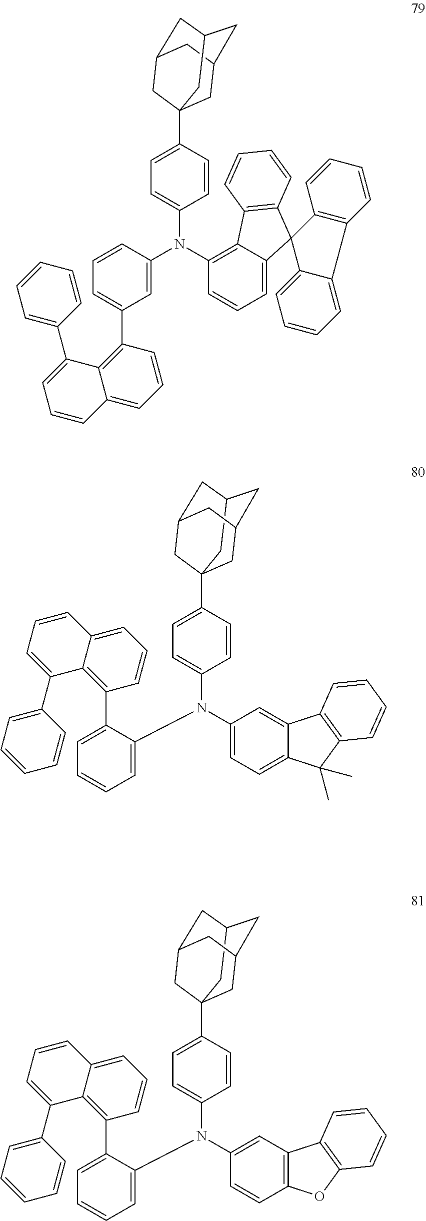 [SMILES: c1ccc(-c2cccc3cccc(-c4cccc(N(c5ccc(C67CC8CC(CC(C8)C6)C7)cc5)c5cccc6c5-c5ccccc5C65c6ccccc6-c6ccccc65)c4)c23)cc1 ; CC1(C)c2ccccc2-c2cc(N(c3ccc(C45CC6CC(CC(C6)C4)C5)cc3)c3ccccc3-c3cccc4cccc(-c5ccccc5)c34)ccc21 ; c1ccc(-c2cccc3cccc(-c4ccccc4N(c4ccc(C56CC7CC(CC(C7)C5)C6)cc4)c4ccc5oc6ccccc6c5c4)c23)cc1]

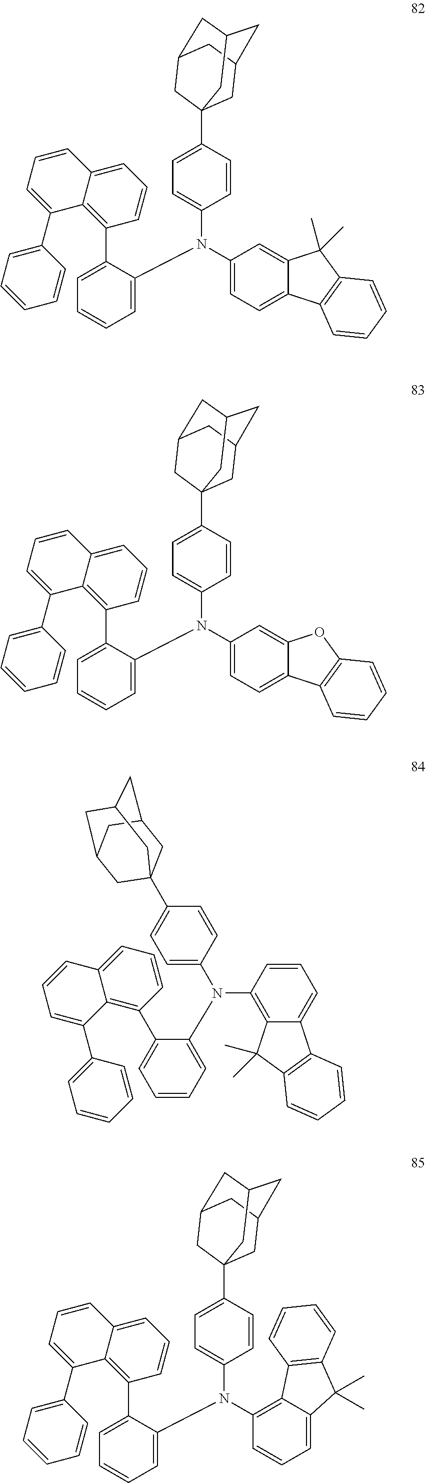 [SMILES: CC1(C)c2ccccc2-c2ccc(N(c3ccc(C45CC6CC(CC(C6)C4)C5)cc3)c3ccccc3-c3cccc4cccc(-c5ccccc5)c34)cc21 ; c1ccc(-c2cccc3cccc(-c4ccccc4N(c4ccc(C56CC7CC(CC(C7)C5)C6)cc4)c4ccc5c(c4)oc4ccccc45)c23)cc1 ; CC1(C)c2ccccc2-c2cccc(N(c3ccc(C45CC6CC(CC(C6)C4)C5)cc3)c3ccccc3-c3cccc4cccc(-c5ccccc5)c34)c21 ; CC1(C)c2ccccc2-c2c(N(c3ccc(C45CC6CC(CC(C6)C4)C5)cc3)c3ccccc3-c3cccc4cccc(-c5ccccc5)c34)cccc21]

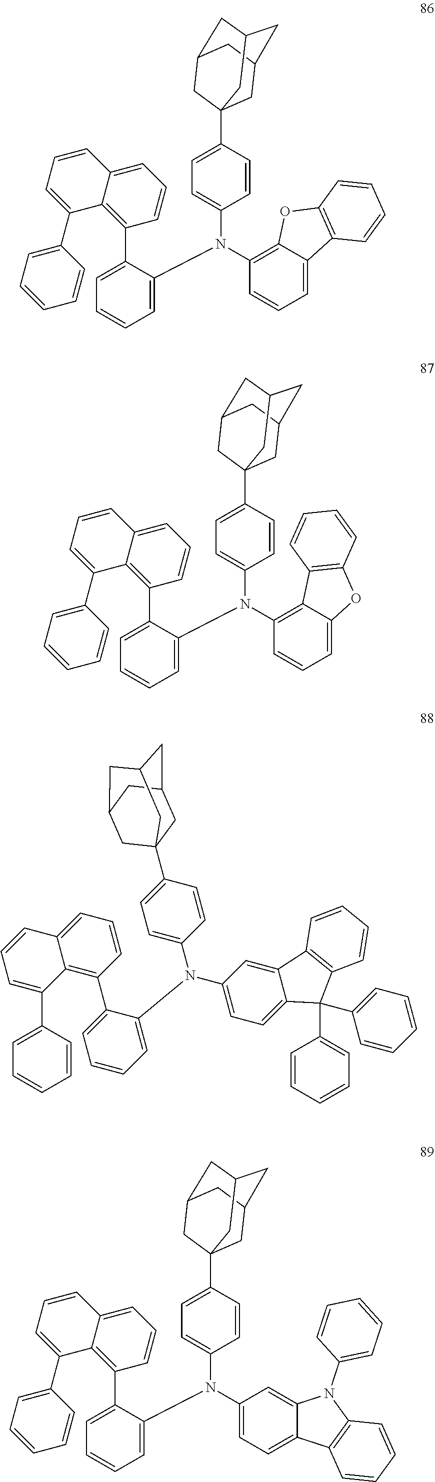 [SMILES: c1ccc(-c2cccc3cccc(-c4ccccc4N(c4ccc(C56CC7CC(CC(C7)C5)C6)cc4)c4cccc5c4oc4ccccc45)c23)cc1 ; c1ccc(-c2cccc3cccc(-c4ccccc4N(c4ccc(C56CC7CC(CC(C7)C5)C6)cc4)c4cccc5oc6ccccc6c45)c23)cc1 ; c1ccc(-c2cccc3cccc(-c4ccccc4N(c4ccc(C56CC7CC(CC(C7)C5)C6)cc4)c4ccc5c(c4)-c4ccccc4C5(c4ccccc4)c4ccccc4)c23)cc1 ; c1ccc(-c2cccc3cccc(-c4ccccc4N(c4ccc(C56CC7CC(CC(C7)C5)C6)cc4)c4ccc5c6ccccc6n(-c6ccccc6)c5c4)c23)cc1]

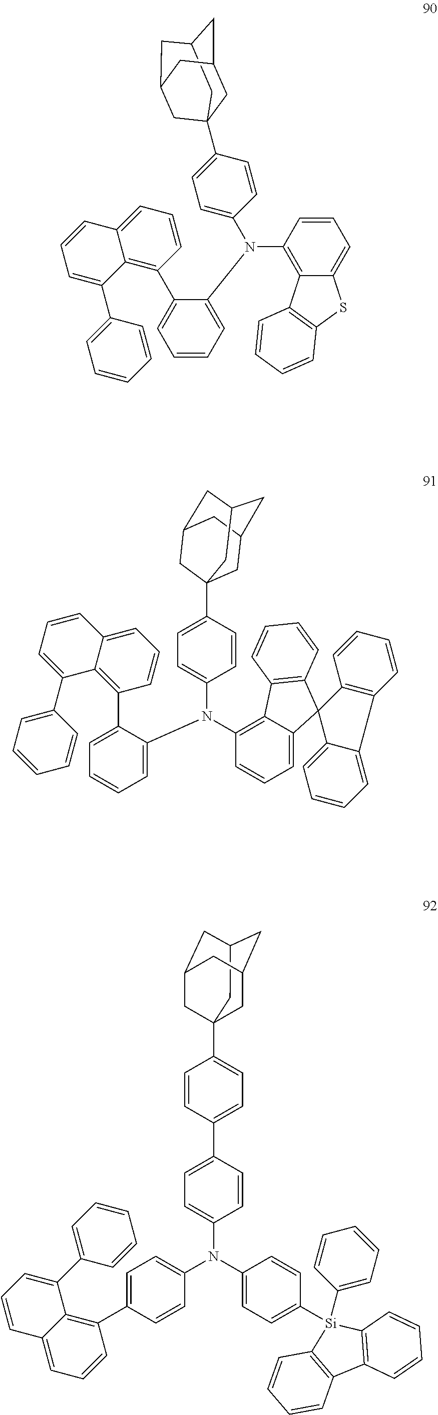 [SMILES: c1ccc(-c2cccc3cccc(-c4ccccc4N(c4ccc(C56CC7CC(CC(C7)C5)C6)cc4)c4cccc5sc6ccccc6c45)c23)cc1 ; c1ccc(-c2cccc3cccc(-c4ccccc4N(c4ccc(C56CC7CC(CC(C7)C5)C6)cc4)c4cccc5c4-c4ccccc4C54c5ccccc5-c5ccccc54)c23)cc1 ; c1ccc(-c2cccc3cccc(-c4ccc(N(c5ccc(-c6ccc(C78CC9CC(CC(C9)C7)C8)cc6)cc5)c5ccc([Si]6(c7ccccc7)c7ccccc7-c7ccccc76)cc5)cc4)c23)cc1]

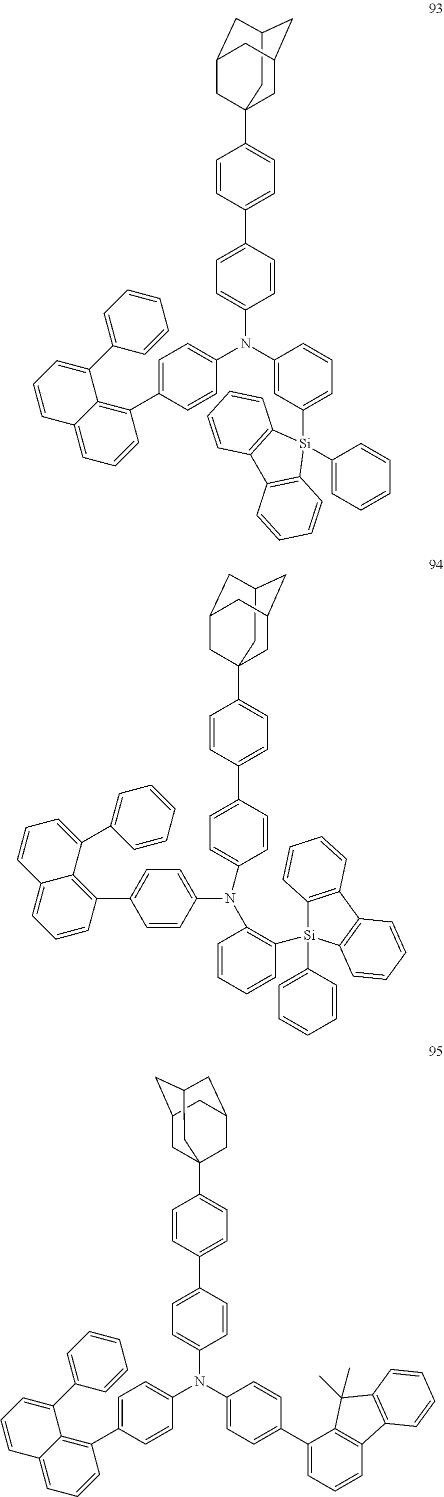

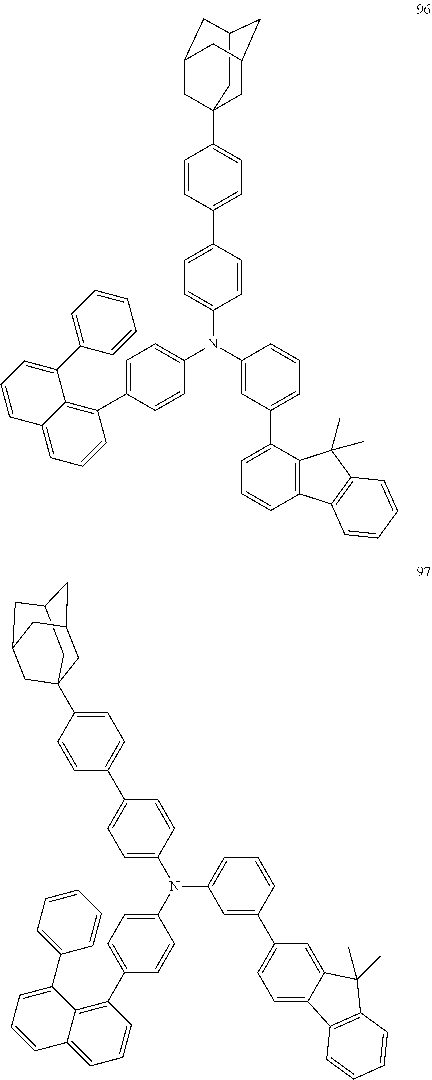 [SMILES: CC1(C)c2ccccc2-c2cccc(-c3cccc(N(c4ccc(-c5ccc(C67CC8CC(CC(C8)C6)C7)cc5)cc4)c4ccc(-c5cccc6cccc(-c7ccccc7)c56)cc4)c3)c21 ; CC1(C)c2ccccc2-c2ccc(-c3cccc(N(c4ccc(-c5ccc(C67CC8CC(CC(C8)C6)C7)cc5)cc4)c4ccc(-c5cccc6cccc(-c7ccccc7)c56)cc4)c3)cc21]

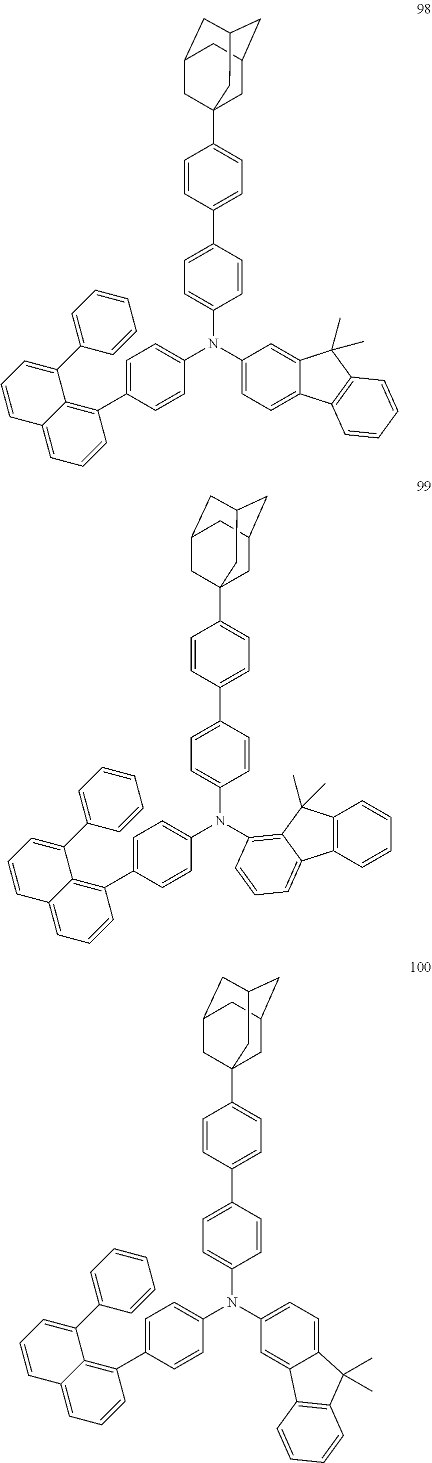 [SMILES: CC1(C)c2ccccc2-c2ccc(N(c3ccc(-c4ccc(C56CC7CC(CC(C7)C5)C6)cc4)cc3)c3ccc(-c4cccc5cccc(-c6ccccc6)c45)cc3)cc21 ; CC1(C)c2ccccc2-c2cccc(N(c3ccc(-c4ccc(C56CC7CC(CC(C7)C5)C6)cc4)cc3)c3ccc(-c4cccc5cccc(-c6ccccc6)c45)cc3)c21 ; CC1(C)c2ccccc2-c2cc(N(c3ccc(-c4ccc(C56CC7CC(CC(C7)C5)C6)cc4)cc3)c3ccc(-c4cccc5cccc(-c6ccccc6)c45)cc3)ccc21]

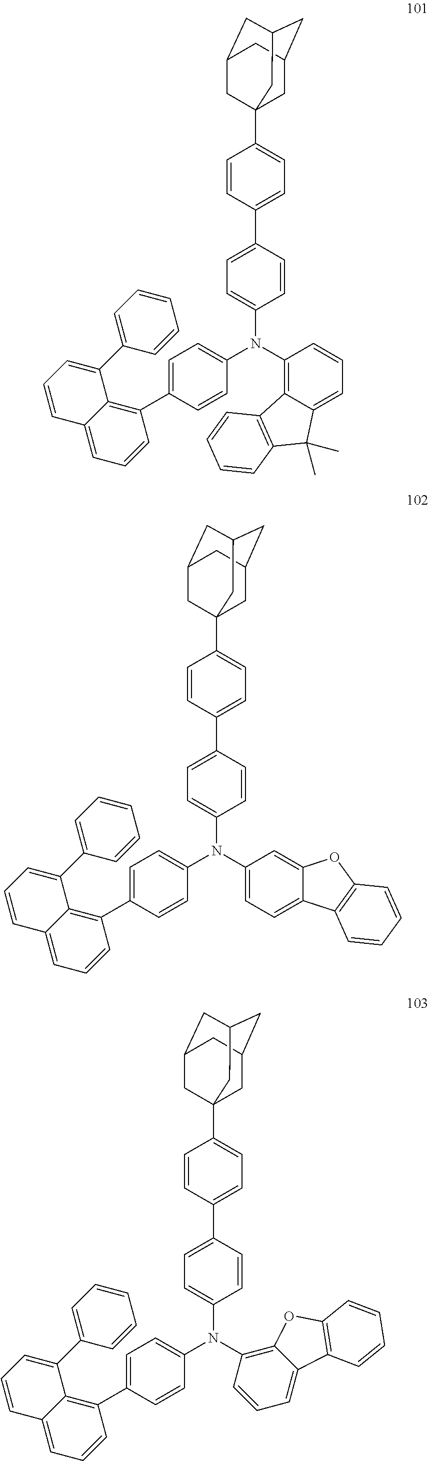 [SMILES: CC1(C)c2ccccc2-c2c(N(c3ccc(-c4ccc(C56CC7CC(CC(C7)C5)C6)cc4)cc3)c3ccc(-c4cccc5cccc(-c6ccccc6)c45)cc3)cccc21 ; c1ccc(-c2cccc3cccc(-c4ccc(N(c5ccc(-c6ccc(C78CC9CC(CC(C9)C7)C8)cc6)cc5)c5ccc6c(c5)oc5ccccc56)cc4)c23)cc1 ; c1ccc(-c2cccc3cccc(-c4ccc(N(c5ccc(-c6ccc(C78CC9CC(CC(C9)C7)C8)cc6)cc5)c5cccc6c5oc5ccccc56)cc4)c23)cc1]

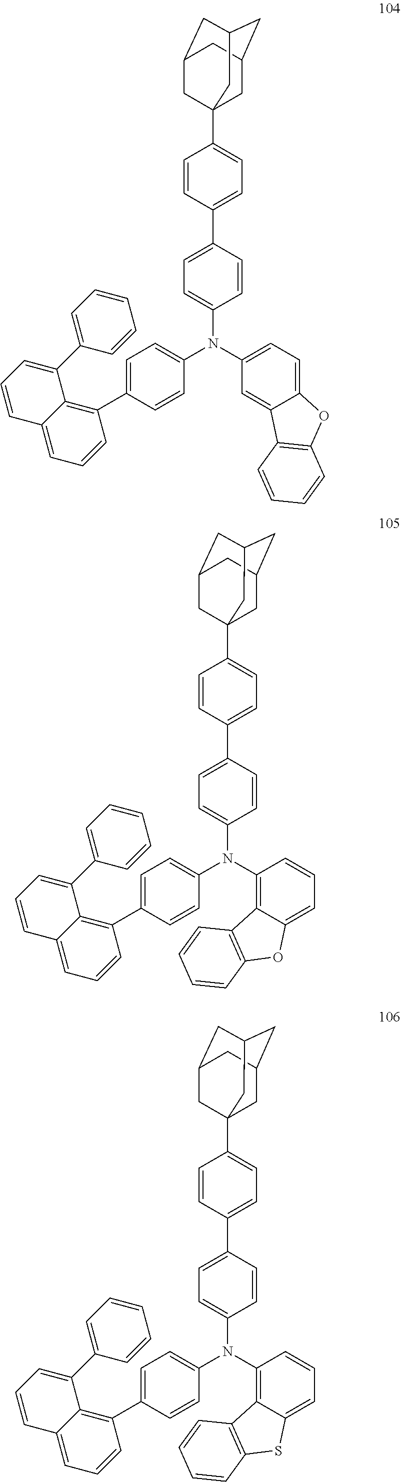

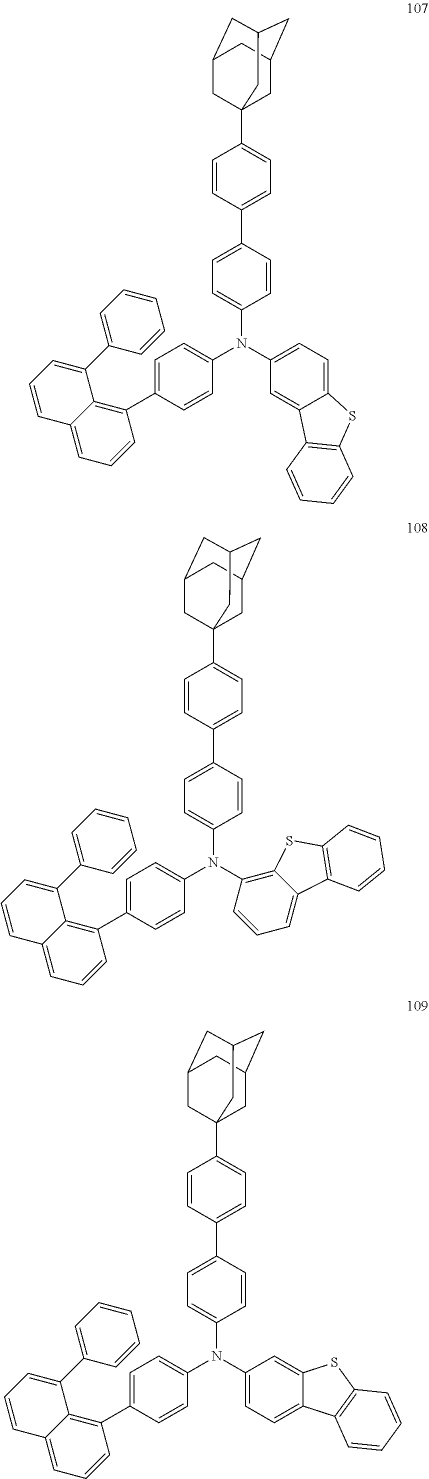 [SMILES: c1ccc(-c2cccc3cccc(-c4ccc(N(c5ccc(-c6ccc(C78CC9CC(CC(C9)C7)C8)cc6)cc5)c5ccc6sc7ccccc7c6c5)cc4)c23)cc1 ; c1ccc(-c2cccc3cccc(-c4ccc(N(c5ccc(-c6ccc(C78CC9CC(CC(C9)C7)C8)cc6)cc5)c5cccc6c5sc5ccccc56)cc4)c23)cc1 ; c1ccc(-c2cccc3cccc(-c4ccc(N(c5ccc(-c6ccc(C78CC9CC(CC(C9)C7)C8)cc6)cc5)c5ccc6c(c5)sc5ccccc56)cc4)c23)cc1]

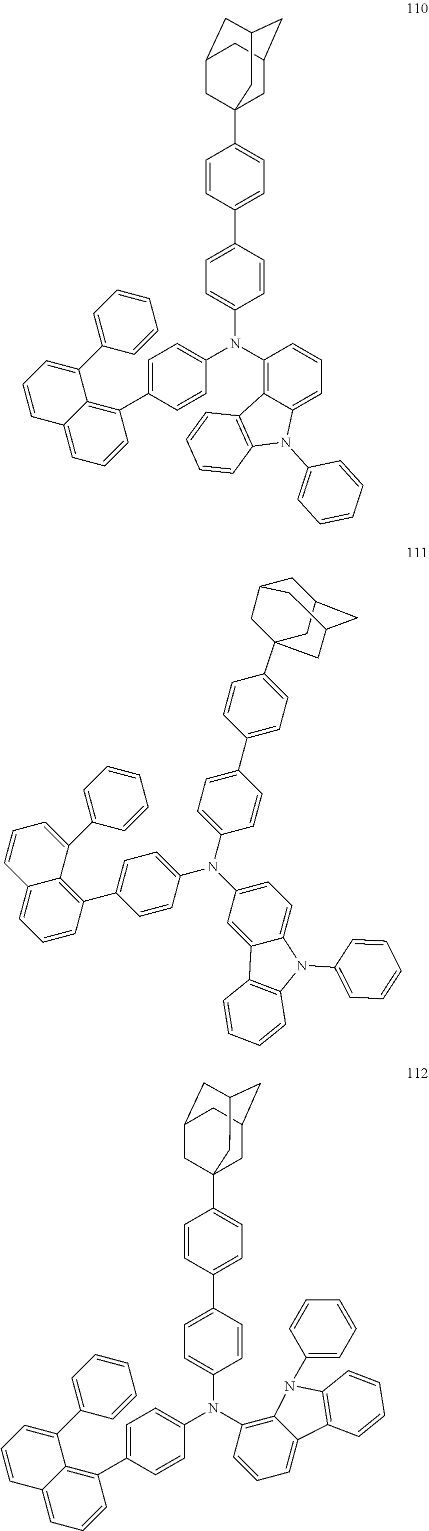

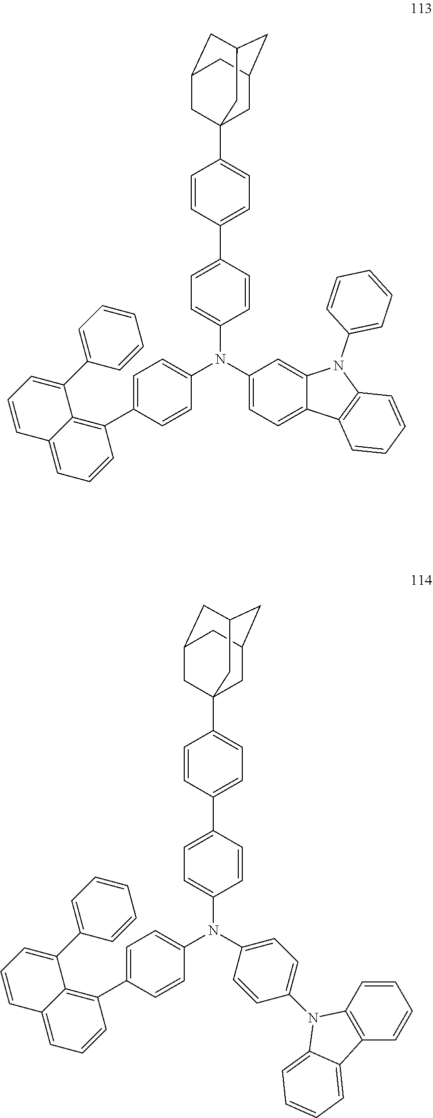 [SMILES: c1ccc(-c2cccc3cccc(-c4ccc(N(c5ccc(-c6ccc(C78CC9CC(CC(C9)C7)C8)cc6)cc5)c5ccc6c7ccccc7n(-c7ccccc7)c6c5)cc4)c23)cc1 ; c1ccc(-c2cccc3cccc(-c4ccc(N(c5ccc(-c6ccc(C78CC9CC(CC(C9)C7)C8)cc6)cc5)c5ccc(-n6c7ccccc7c7ccccc76)cc5)cc4)c23)cc1]

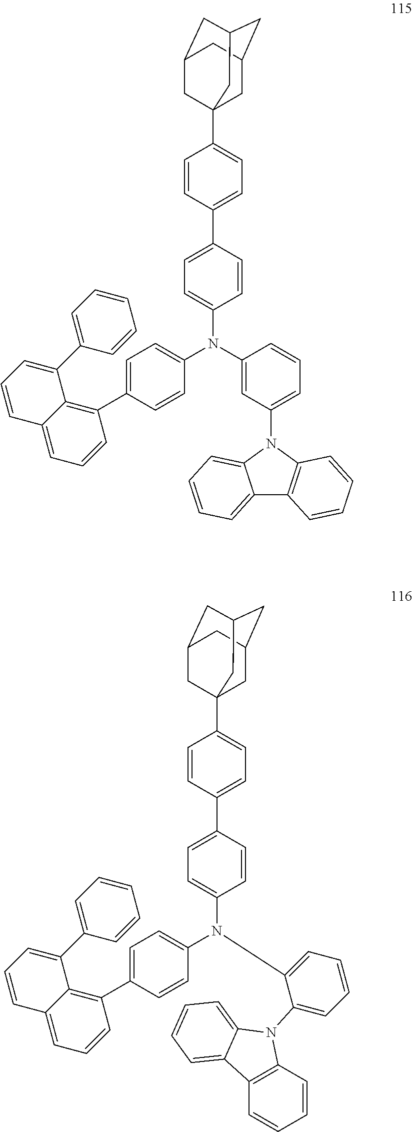 [SMILES: c1ccc(-c2cccc3cccc(-c4ccc(N(c5ccc(-c6ccc(C78CC9CC(CC(C9)C7)C8)cc6)cc5)c5cccc(-n6c7ccccc7c7ccccc76)c5)cc4)c23)cc1 ; c1ccc(-c2cccc3cccc(-c4ccc(N(c5ccc(-c6ccc(C78CC9CC(CC(C9)C7)C8)cc6)cc5)c5ccccc5-n5c6ccccc6c6ccccc65)cc4)c23)cc1]

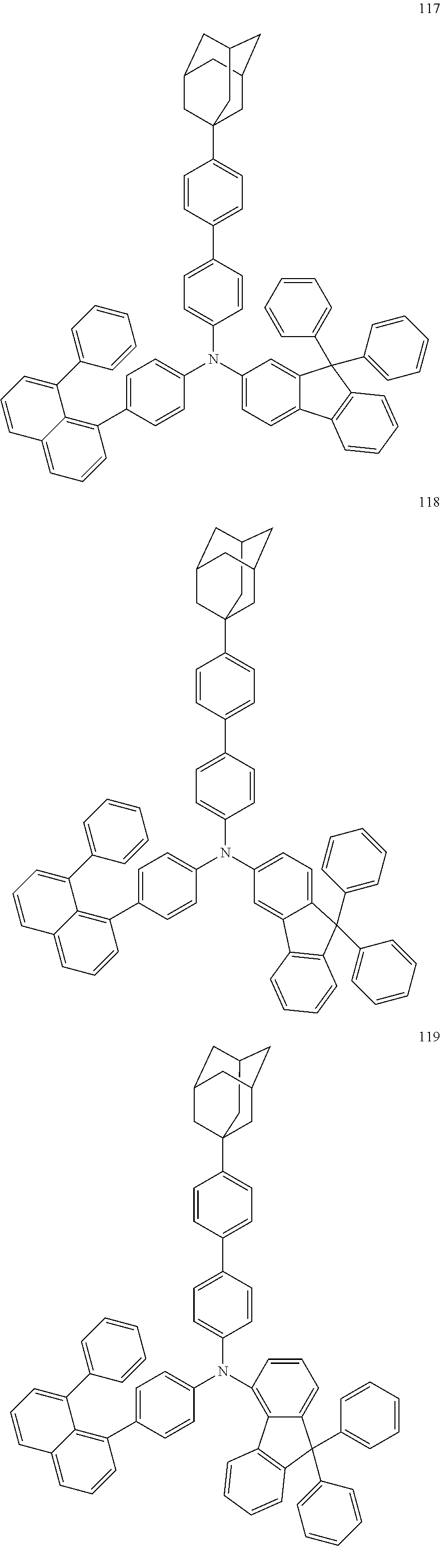

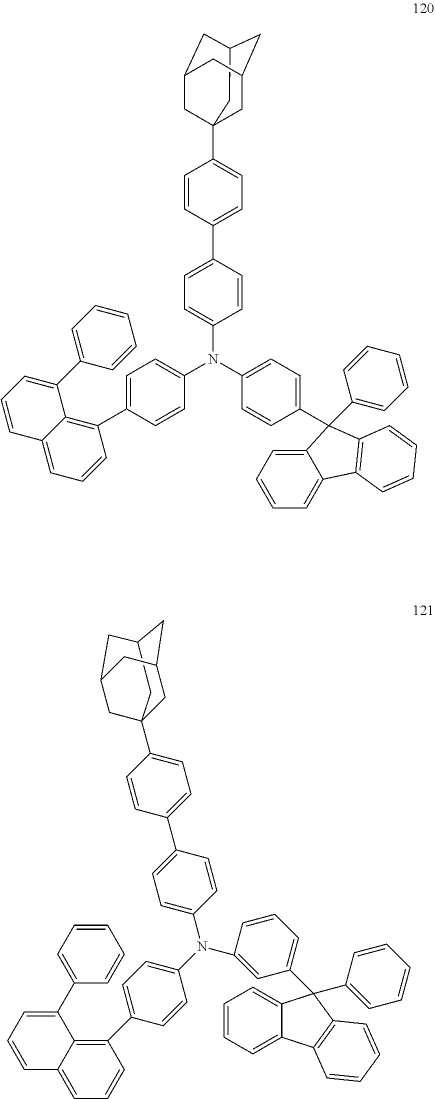 [SMILES: c1ccc(-c2cccc3cccc(-c4ccc(N(c5ccc(-c6ccc(C78CC9CC(CC(C9)C7)C8)cc6)cc5)c5ccc(C6(c7ccccc7)c7ccccc7-c7ccccc76)cc5)cc4)c23)cc1 ; c1ccc(-c2cccc3cccc(-c4ccc(N(c5ccc(-c6ccc(C78CC9CC(CC(C9)C7)C8)cc6)cc5)c5cccc(C6(c7ccccc7)c7ccccc7-c7ccccc76)c5)cc4)c23)cc1]

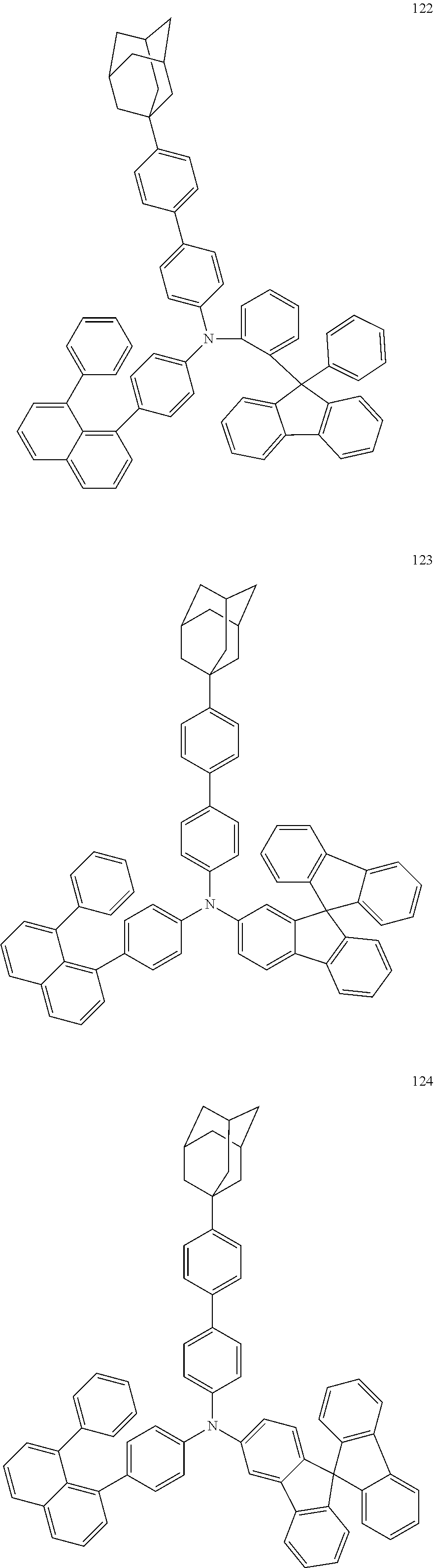 [SMILES: c1ccc(-c2cccc3cccc(-c4ccc(N(c5ccc(-c6ccc(C78CC9CC(CC(C9)C7)C8)cc6)cc5)c5ccccc5C5(c6ccccc6)c6ccccc6-c6ccccc65)cc4)c23)cc1 ; c1ccc(-c2cccc3cccc(-c4ccc(N(c5ccc(-c6ccc(C78CC9CC(CC(C9)C7)C8)cc6)cc5)c5ccc6c(c5)C5(c7ccccc7-c7ccccc75)c5ccccc5-6)cc4)c23)cc1 ; c1ccc(-c2cccc3cccc(-c4ccc(N(c5ccc(-c6ccc(C78CC9CC(CC(C9)C7)C8)cc6)cc5)c5ccc6c(c5)-c5ccccc5C65c6ccccc6-c6ccccc65)cc4)c23)cc1]

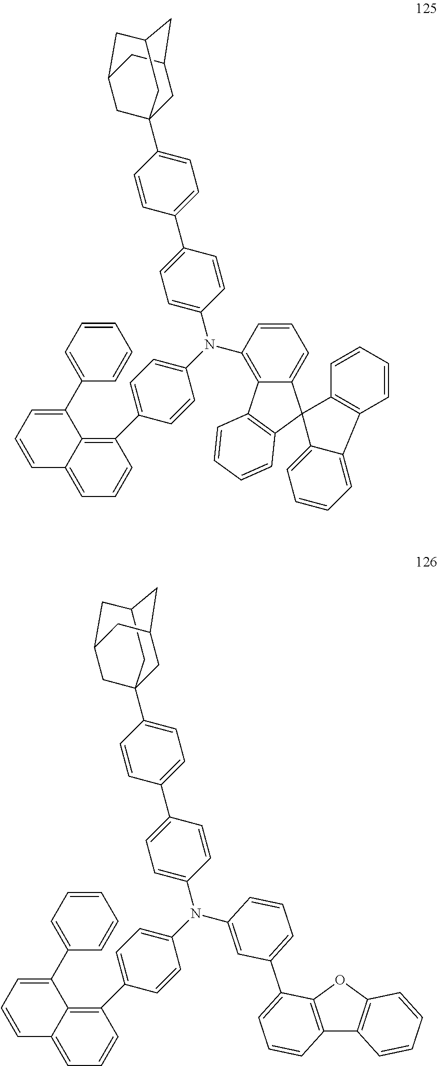 [SMILES: c1ccc(-c2cccc3cccc(-c4ccc(N(c5ccc(-c6ccc(C78CC9CC(CC(C9)C7)C8)cc6)cc5)c5cccc6c5-c5ccccc5C65c6ccccc6-c6ccccc65)cc4)c23)cc1 ; c1ccc(-c2cccc3cccc(-c4ccc(N(c5ccc(-c6ccc(C78CC9CC(CC(C9)C7)C8)cc6)cc5)c5cccc(-c6cccc7c6oc6ccccc67)c5)cc4)c23)cc1]

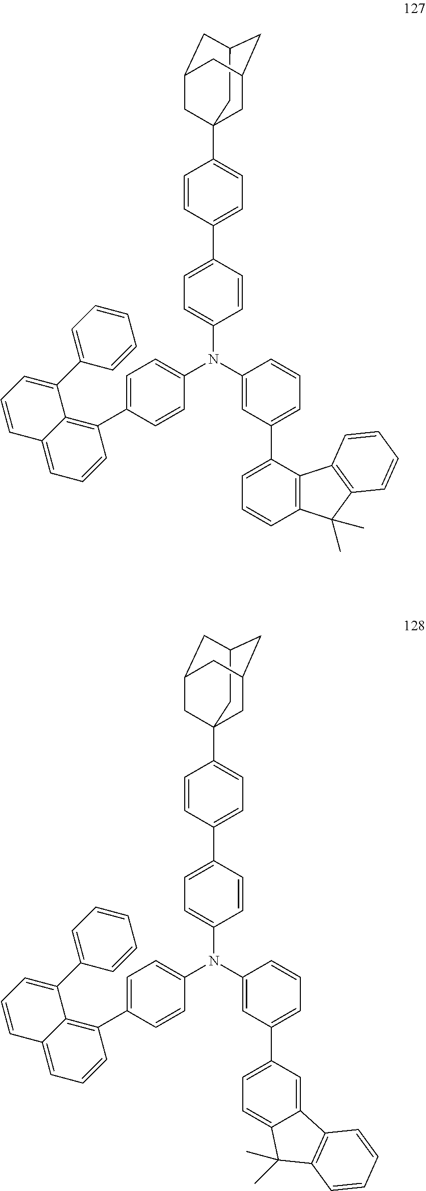 [SMILES: CC1(C)c2ccccc2-c2c(-c3cccc(N(c4ccc(-c5ccc(C67CC8CC(CC(C8)C6)C7)cc5)cc4)c4ccc(-c5cccc6cccc(-c7ccccc7)c56)cc4)c3)cccc21 ; CC1(C)c2ccccc2-c2cc(-c3cccc(N(c4ccc(-c5ccc(C67CC8CC(CC(C8)C6)C7)cc5)cc4)c4ccc(-c5cccc6cccc(-c7ccccc7)c56)cc4)c3)ccc21]

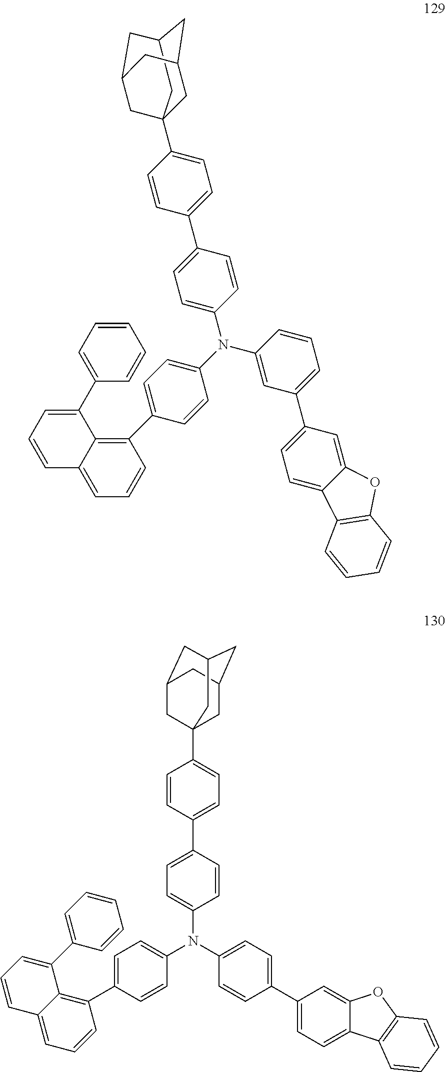 [SMILES: c1ccc(-c2cccc3cccc(-c4ccc(N(c5ccc(-c6ccc(C78CC9CC(CC(C9)C7)C8)cc6)cc5)c5cccc(-c6ccc7c(c6)oc6ccccc67)c5)cc4)c23)cc1 ; c1ccc(-c2cccc3cccc(-c4ccc(N(c5ccc(-c6ccc(C78CC9CC(CC(C9)C7)C8)cc6)cc5)c5ccc(-c6ccc7c(c6)oc6ccccc67)cc5)cc4)c23)cc1]

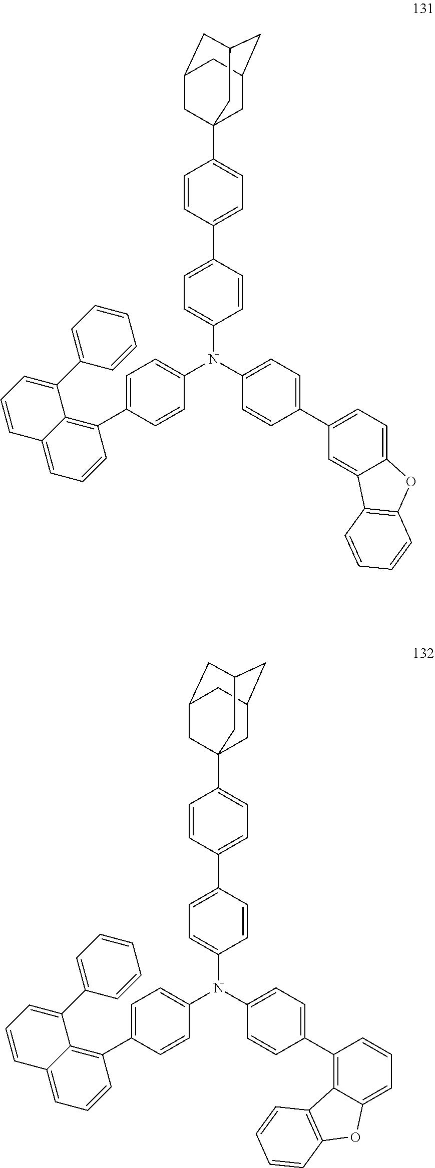 [SMILES: c1ccc(-c2cccc3cccc(-c4ccc(N(c5ccc(-c6ccc(C78CC9CC(CC(C9)C7)C8)cc6)cc5)c5ccc(-c6ccc7oc8ccccc8c7c6)cc5)cc4)c23)cc1 ; c1ccc(-c2cccc3cccc(-c4ccc(N(c5ccc(-c6ccc(C78CC9CC(CC(C9)C7)C8)cc6)cc5)c5ccc(-c6cccc7oc8ccccc8c67)cc5)cc4)c23)cc1]

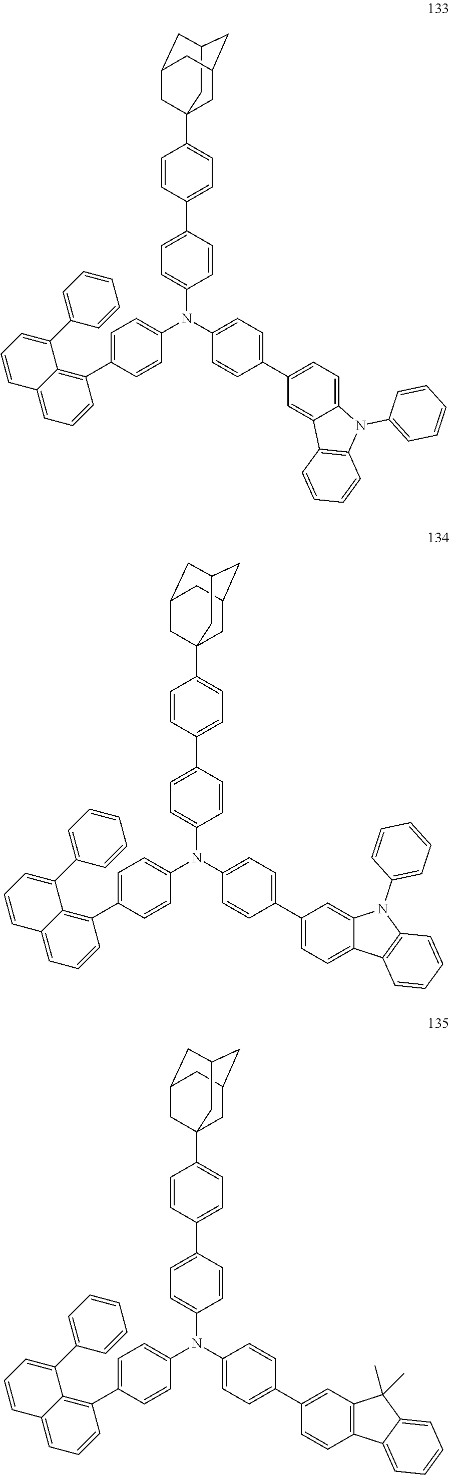 [SMILES: c1ccc(-c2cccc3cccc(-c4ccc(N(c5ccc(-c6ccc(C78CC9CC(CC(C9)C7)C8)cc6)cc5)c5ccc(-c6ccc7c(c6)c6ccccc6n7-c6ccccc6)cc5)cc4)c23)cc1 ; c1ccc(-c2cccc3cccc(-c4ccc(N(c5ccc(-c6ccc(C78CC9CC(CC(C9)C7)C8)cc6)cc5)c5ccc(-c6ccc7c8ccccc8n(-c8ccccc8)c7c6)cc5)cc4)c23)cc1 ; CC1(C)c2ccccc2-c2ccc(-c3ccc(N(c4ccc(-c5ccc(C67CC8CC(CC(C8)C6)C7)cc5)cc4)c4ccc(-c5cccc6cccc(-c7ccccc7)c56)cc4)cc3)cc21]

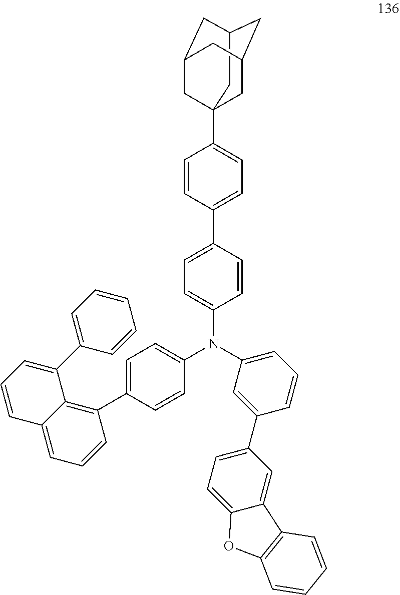 [SMILES: c1ccc(-c2cccc3cccc(-c4ccc(N(c5ccc(-c6ccc(C78CC9CC(CC(C9)C7)C8)cc6)cc5)c5cccc(-c6ccc7oc8ccccc8c7c6)c5)cc4)c23)cc1]

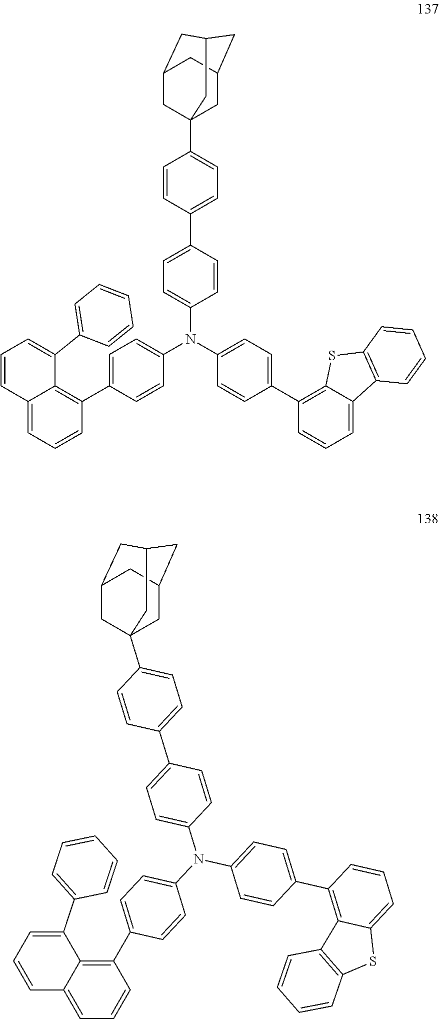 [SMILES: c1ccc(-c2cccc3cccc(-c4ccc(N(c5ccc(-c6ccc(C78CC9CC(CC(C9)C7)C8)cc6)cc5)c5ccc(-c6cccc7c6sc6ccccc67)cc5)cc4)c23)cc1 ; c1ccc(-c2cccc3cccc(-c4ccc(N(c5ccc(-c6ccc(C78CC9CC(CC(C9)C7)C8)cc6)cc5)c5ccc(-c6cccc7sc8ccccc8c67)cc5)cc4)c23)cc1]

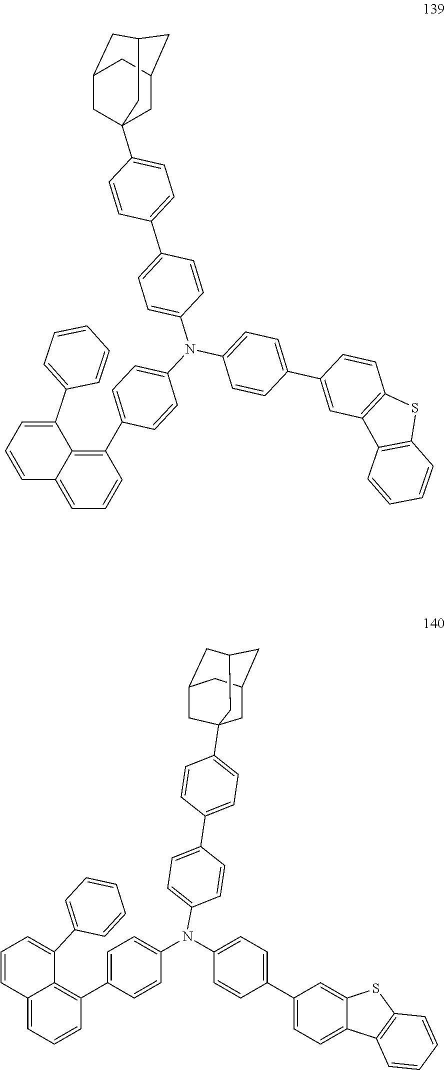 [SMILES: c1ccc(-c2cccc3cccc(-c4ccc(N(c5ccc(-c6ccc(C78CC9CC(CC(C9)C7)C8)cc6)cc5)c5ccc(-c6ccc7sc8ccccc8c7c6)cc5)cc4)c23)cc1 ; c1ccc(-c2cccc3cccc(-c4ccc(N(c5ccc(-c6ccc(C78CC9CC(CC(C9)C7)C8)cc6)cc5)c5ccc(-c6ccc7c(c6)sc6ccccc67)cc5)cc4)c23)cc1]

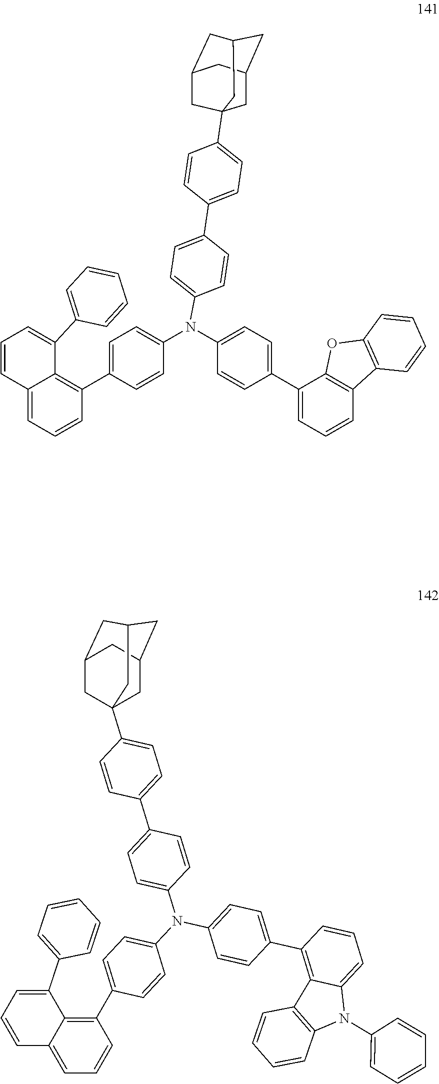 [SMILES: c1ccc(-c2cccc3cccc(-c4ccc(N(c5ccc(-c6ccc(C78CC9CC(CC(C9)C7)C8)cc6)cc5)c5ccc(-c6cccc7c6oc6ccccc67)cc5)cc4)c23)cc1 ; c1ccc(-c2cccc3cccc(-c4ccc(N(c5ccc(-c6ccc(C78CC9CC(CC(C9)C7)C8)cc6)cc5)c5ccc(-c6cccc7c6c6ccccc6n7-c6ccccc6)cc5)cc4)c23)cc1]

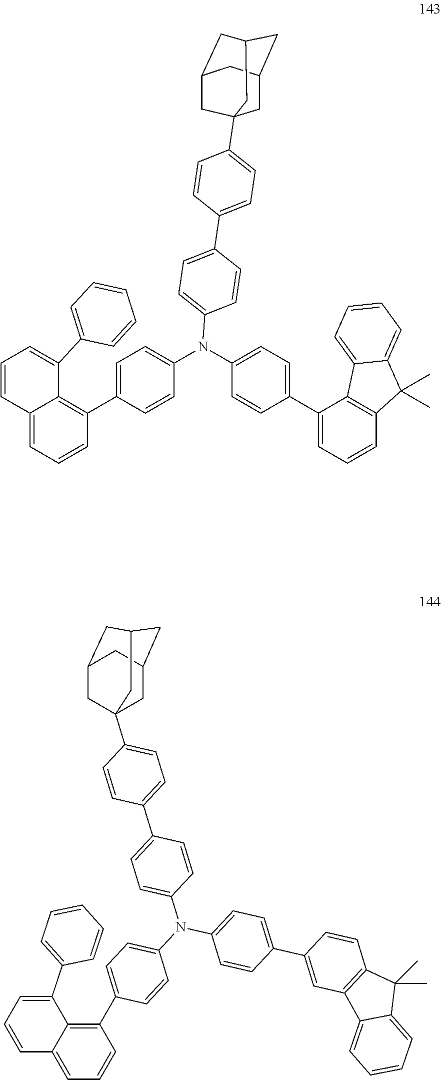 [SMILES: CC1(C)c2ccccc2-c2c(-c3ccc(N(c4ccc(-c5ccc(C67CC8CC(CC(C8)C6)C7)cc5)cc4)c4ccc(-c5cccc6cccc(-c7ccccc7)c56)cc4)cc3)cccc21 ; CC1(C)c2ccccc2-c2cc(-c3ccc(N(c4ccc(-c5ccc(C67CC8CC(CC(C8)C6)C7)cc5)cc4)c4ccc(-c5cccc6cccc(-c7ccccc7)c56)cc4)cc3)ccc21]

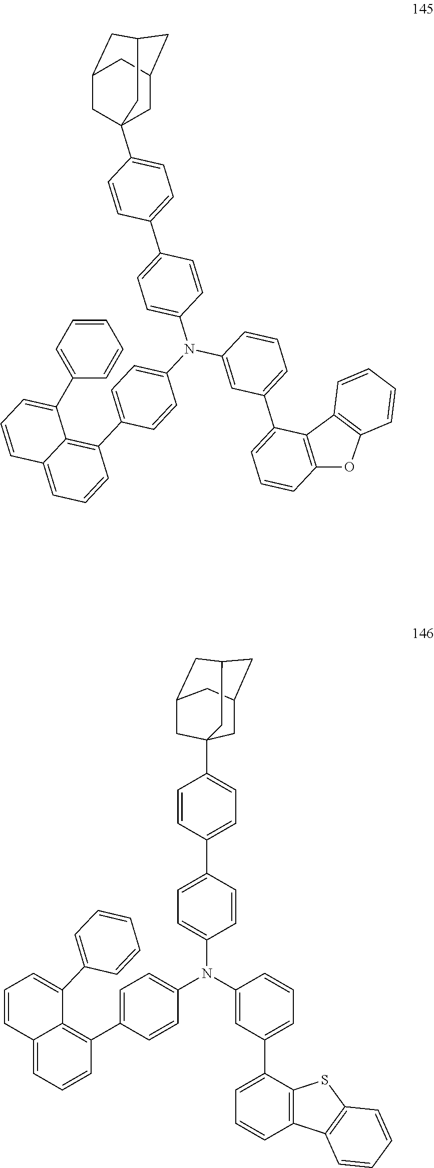 [SMILES: c1ccc(-c2cccc3cccc(-c4ccc(N(c5ccc(-c6ccc(C78CC9CC(CC(C9)C7)C8)cc6)cc5)c5cccc(-c6cccc7oc8ccccc8c67)c5)cc4)c23)cc1 ; c1ccc(-c2cccc3cccc(-c4ccc(N(c5ccc(-c6ccc(C78CC9CC(CC(C9)C7)C8)cc6)cc5)c5cccc(-c6cccc7c6sc6ccccc67)c5)cc4)c23)cc1]

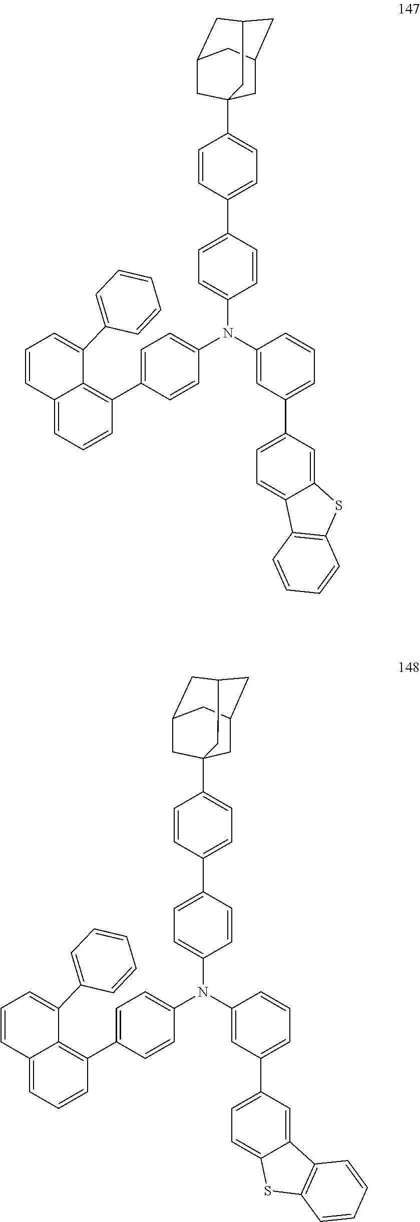 [SMILES: c1ccc(-c2cccc3cccc(-c4ccc(N(c5ccc(-c6ccc(C78CC9CC(CC(C9)C7)C8)cc6)cc5)c5cccc(-c6ccc7c(c6)sc6ccccc67)c5)cc4)c23)cc1 ; c1ccc(-c2cccc3cccc(-c4ccc(N(c5ccc(-c6ccc(C78CC9CC(CC(C9)C7)C8)cc6)cc5)c5cccc(-c6ccc7sc8ccccc8c7c6)c5)cc4)c23)cc1]

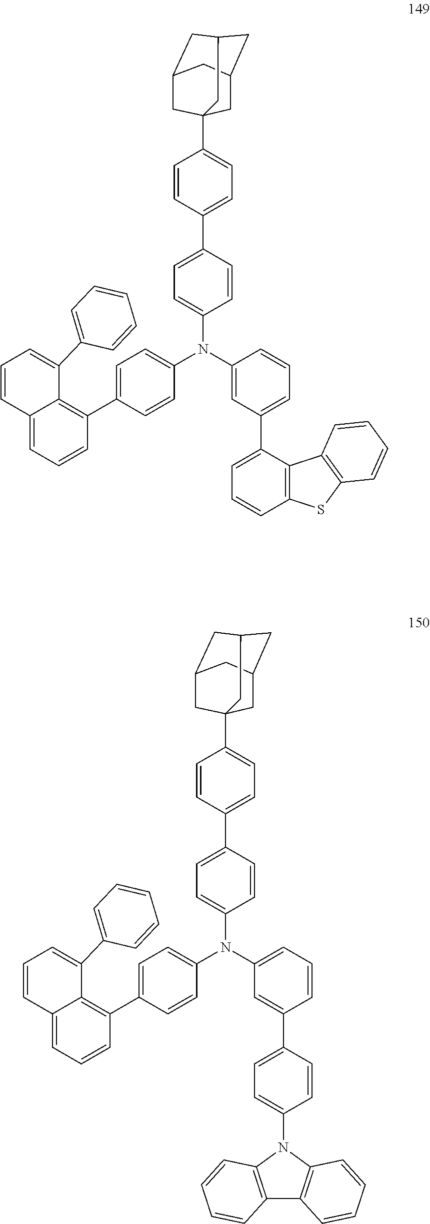 [SMILES: c1ccc(-c2cccc3cccc(-c4ccc(N(c5ccc(-c6ccc(C78CC9CC(CC(C9)C7)C8)cc6)cc5)c5cccc(-c6cccc7sc8ccccc8c67)c5)cc4)c23)cc1 ; c1ccc(-c2cccc3cccc(-c4ccc(N(c5ccc(-c6ccc(C78CC9CC(CC(C9)C7)C8)cc6)cc5)c5cccc(-c6ccc(-n7c8ccccc8c8ccccc87)cc6)c5)cc4)c23)cc1]

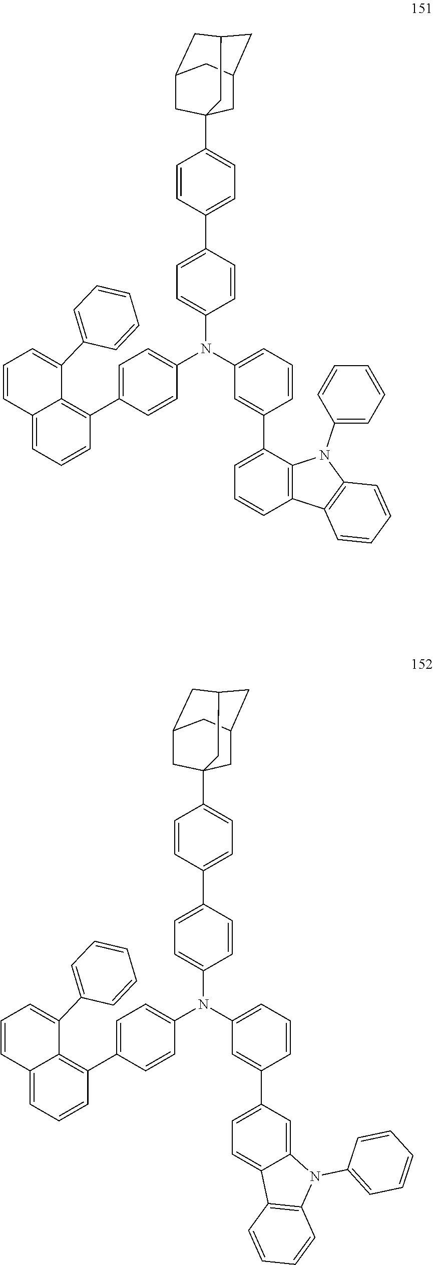 [SMILES: c1ccc(-c2cccc3cccc(-c4ccc(N(c5ccc(-c6ccc(C78CC9CC(CC(C9)C7)C8)cc6)cc5)c5cccc(-c6cccc7c8ccccc8n(-c8ccccc8)c67)c5)cc4)c23)cc1 ; c1ccc(-c2cccc3cccc(-c4ccc(N(c5ccc(-c6ccc(C78CC9CC(CC(C9)C7)C8)cc6)cc5)c5cccc(-c6ccc7c8ccccc8n(-c8ccccc8)c7c6)c5)cc4)c23)cc1]

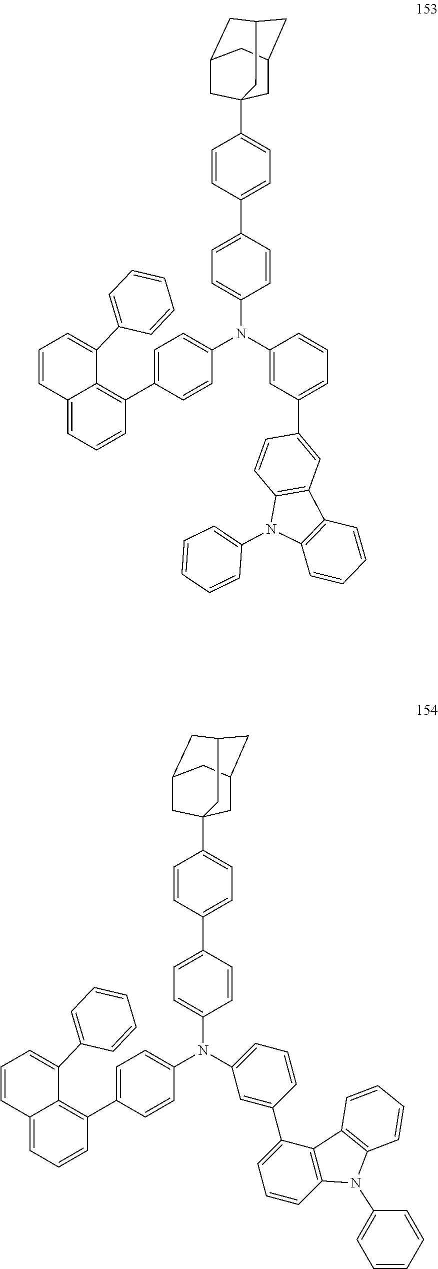 [SMILES: c1ccc(-c2cccc3cccc(-c4ccc(N(c5ccc(-c6ccc(C78CC9CC(CC(C9)C7)C8)cc6)cc5)c5cccc(-c6ccc7c(c6)c6ccccc6n7-c6ccccc6)c5)cc4)c23)cc1 ; c1ccc(-c2cccc3cccc(-c4ccc(N(c5ccc(-c6ccc(C78CC9CC(CC(C9)C7)C8)cc6)cc5)c5cccc(-c6cccc7c6c6ccccc6n7-c6ccccc6)c5)cc4)c23)cc1]

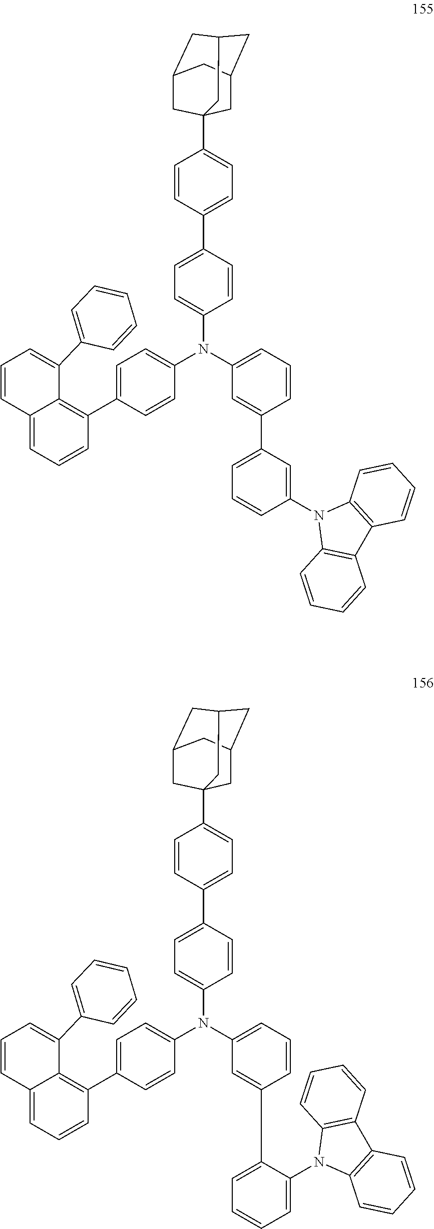 [SMILES: c1ccc(-c2cccc3cccc(-c4ccc(N(c5ccc(-c6ccc(C78CC9CC(CC(C9)C7)C8)cc6)cc5)c5cccc(-c6cccc(-n7c8ccccc8c8ccccc87)c6)c5)cc4)c23)cc1 ; c1ccc(-c2cccc3cccc(-c4ccc(N(c5ccc(-c6ccc(C78CC9CC(CC(C9)C7)C8)cc6)cc5)c5cccc(-c6ccccc6-n6c7ccccc7c7ccccc76)c5)cc4)c23)cc1]

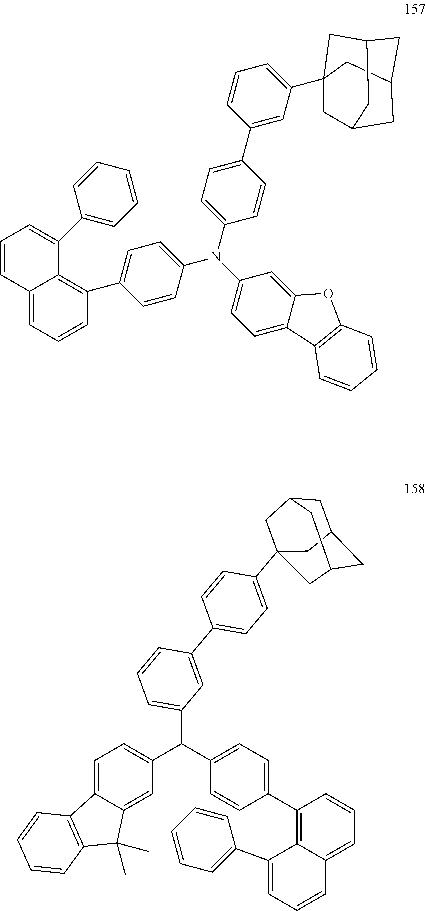

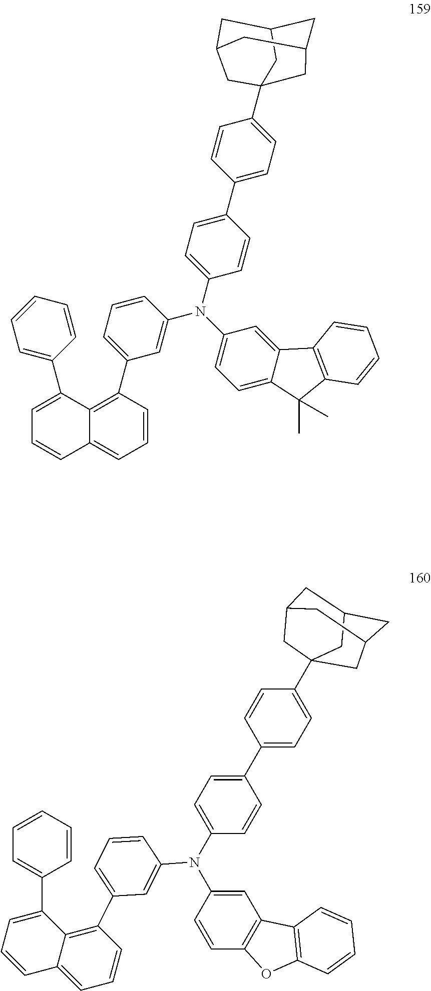 [SMILES: CC1(C)c2ccccc2-c2cc(N(c3ccc(-c4ccc(C56CC7CC(CC(C7)C5)C6)cc4)cc3)c3cccc(-c4cccc5cccc(-c6ccccc6)c45)c3)ccc21 ; c1ccc(-c2cccc3cccc(-c4cccc(N(c5ccc(-c6ccc(C78CC9CC(CC(C9)C7)C8)cc6)cc5)c5ccc6oc7ccccc7c6c5)c4)c23)cc1]

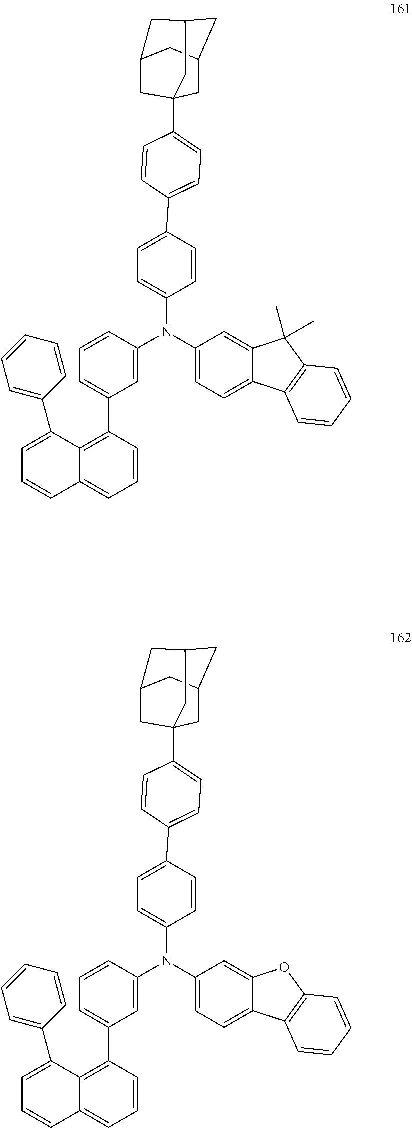 [SMILES: CC1(C)c2ccccc2-c2ccc(N(c3ccc(-c4ccc(C56CC7CC(CC(C7)C5)C6)cc4)cc3)c3cccc(-c4cccc5cccc(-c6ccccc6)c45)c3)cc21 ; c1ccc(-c2cccc3cccc(-c4cccc(N(c5ccc(-c6ccc(C78CC9CC(CC(C9)C7)C8)cc6)cc5)c5ccc6c(c5)oc5ccccc56)c4)c23)cc1]

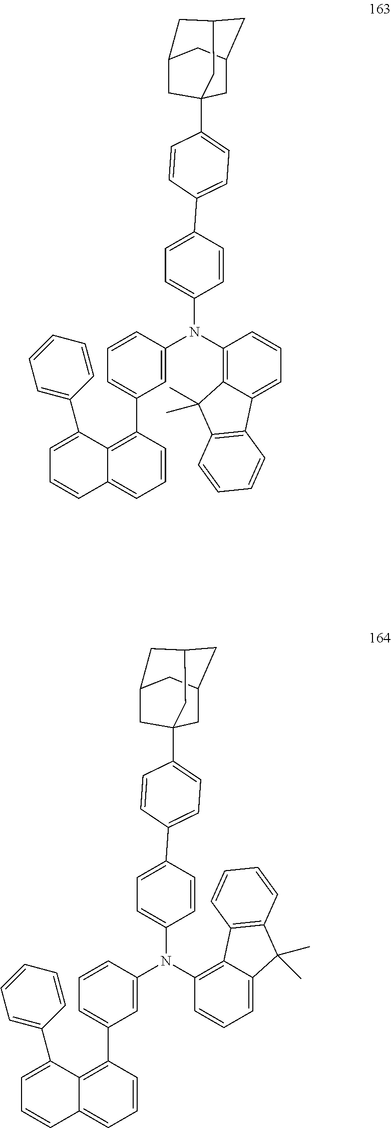 [SMILES: CC1(C)c2ccccc2-c2cccc(N(c3ccc(-c4ccc(C56CC7CC(CC(C7)C5)C6)cc4)cc3)c3cccc(-c4cccc5cccc(-c6ccccc6)c45)c3)c21 ; CC1(C)c2ccccc2-c2c(N(c3ccc(-c4ccc(C56CC7CC(CC(C7)C5)C6)cc4)cc3)c3cccc(-c4cccc5cccc(-c6ccccc6)c45)c3)cccc21]

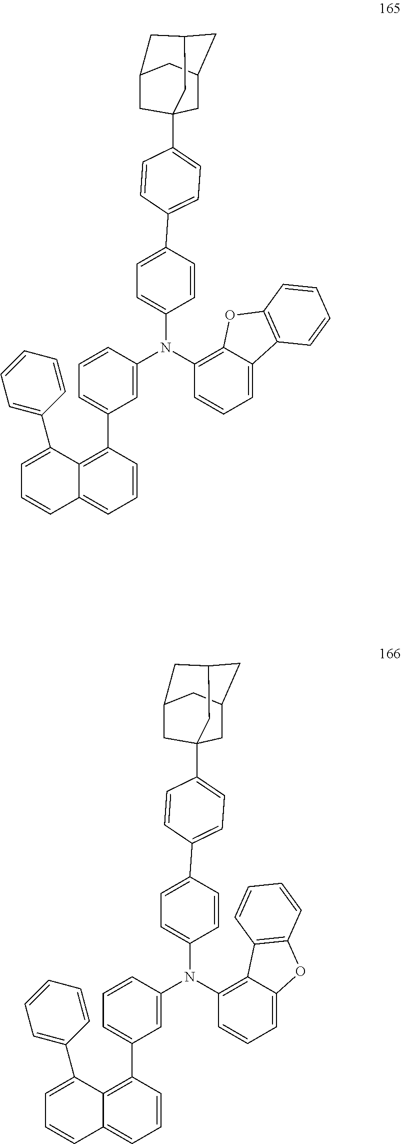 [SMILES: c1ccc(-c2cccc3cccc(-c4cccc(N(c5ccc(-c6ccc(C78CC9CC(CC(C9)C7)C8)cc6)cc5)c5cccc6c5oc5ccccc56)c4)c23)cc1 ; c1ccc(-c2cccc3cccc(-c4cccc(N(c5ccc(-c6ccc(C78CC9CC(CC(C9)C7)C8)cc6)cc5)c5cccc6oc7ccccc7c56)c4)c23)cc1]

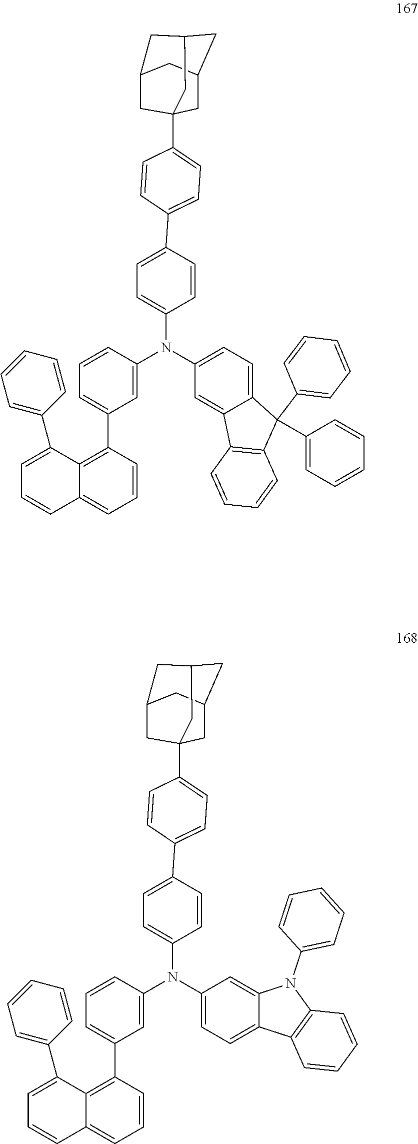 [SMILES: c1ccc(-c2cccc3cccc(-c4cccc(N(c5ccc(-c6ccc(C78CC9CC(CC(C9)C7)C8)cc6)cc5)c5ccc6c(c5)-c5ccccc5C6(c5ccccc5)c5ccccc5)c4)c23)cc1 ; c1ccc(-c2cccc3cccc(-c4cccc(N(c5ccc(-c6ccc(C78CC9CC(CC(C9)C7)C8)cc6)cc5)c5ccc6c7ccccc7n(-c7ccccc7)c6c5)c4)c23)cc1]

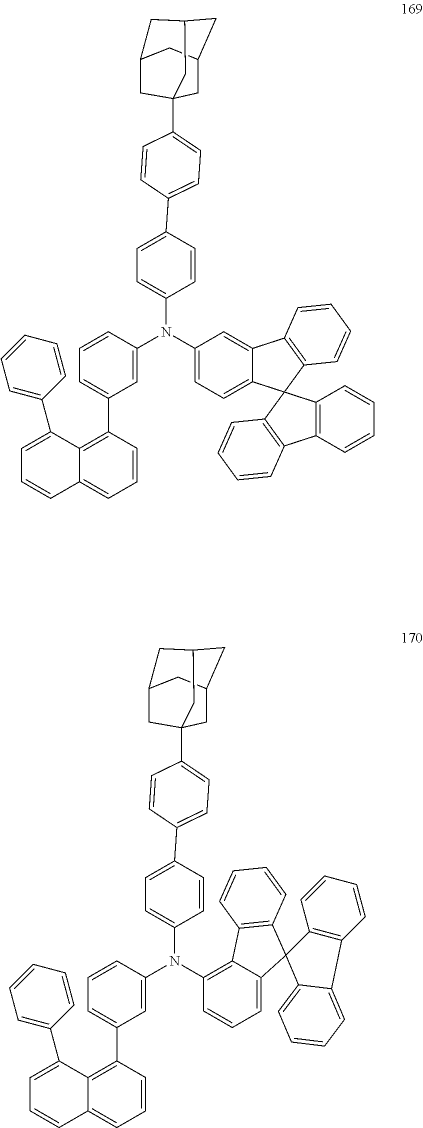 [SMILES: c1ccc(-c2cccc3cccc(-c4cccc(N(c5ccc(-c6ccc(C78CC9CC(CC(C9)C7)C8)cc6)cc5)c5ccc6c(c5)-c5ccccc5C65c6ccccc6-c6ccccc65)c4)c23)cc1 ; c1ccc(-c2cccc3cccc(-c4cccc(N(c5ccc(-c6ccc(C78CC9CC(CC(C9)C7)C8)cc6)cc5)c5cccc6c5-c5ccccc5C65c6ccccc6-c6ccccc65)c4)c23)cc1]

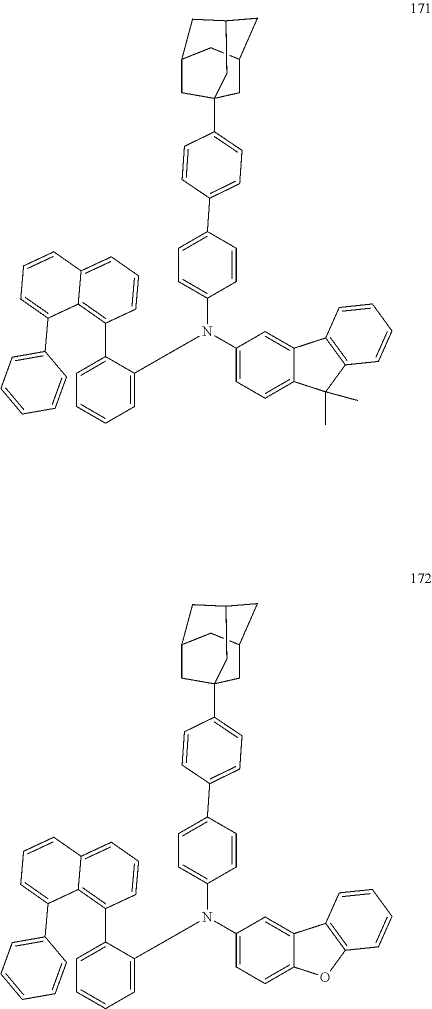 [SMILES: CC1(C)c2ccccc2-c2cc(N(c3ccc(-c4ccc(C56CC7CC(CC(C7)C5)C6)cc4)cc3)c3ccccc3-c3cccc4cccc(-c5ccccc5)c34)ccc21 ; c1ccc(-c2cccc3cccc(-c4ccccc4N(c4ccc(-c5ccc(C67CC8CC(CC(C8)C6)C7)cc5)cc4)c4ccc5oc6ccccc6c5c4)c23)cc1]

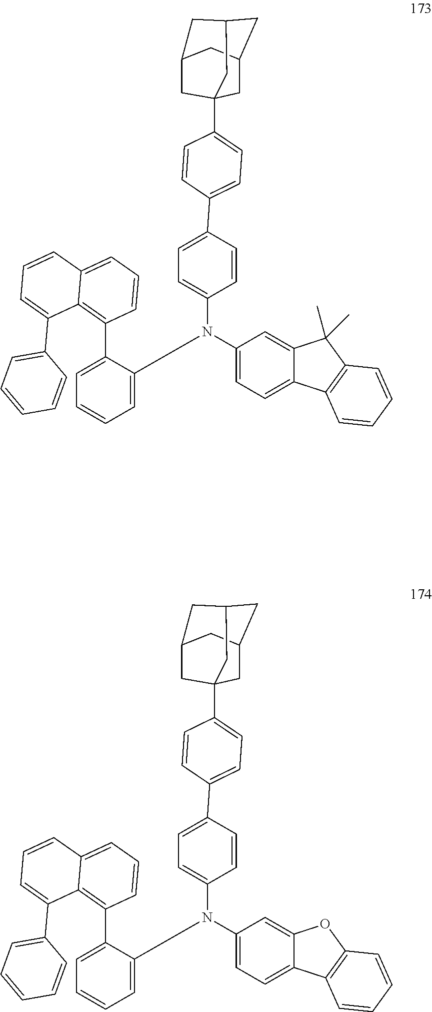 [SMILES: CC1(C)c2ccccc2-c2ccc(N(c3ccc(-c4ccc(C56CC7CC(CC(C7)C5)C6)cc4)cc3)c3ccccc3-c3cccc4cccc(-c5ccccc5)c34)cc21 ; c1ccc(-c2cccc3cccc(-c4ccccc4N(c4ccc(-c5ccc(C67CC8CC(CC(C8)C6)C7)cc5)cc4)c4ccc5c(c4)oc4ccccc45)c23)cc1]

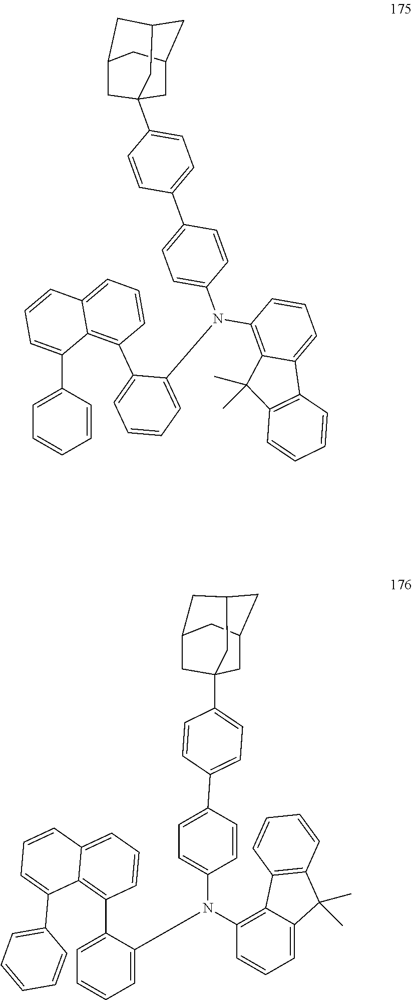 [SMILES: CC1(C)c2ccccc2-c2cccc(N(c3ccc(-c4ccc(C56CC7CC(CC(C7)C5)C6)cc4)cc3)c3ccccc3-c3cccc4cccc(-c5ccccc5)c34)c21 ; CC1(C)c2ccccc2-c2c(N(c3ccc(-c4ccc(C56CC7CC(CC(C7)C5)C6)cc4)cc3)c3ccccc3-c3cccc4cccc(-c5ccccc5)c34)cccc21]

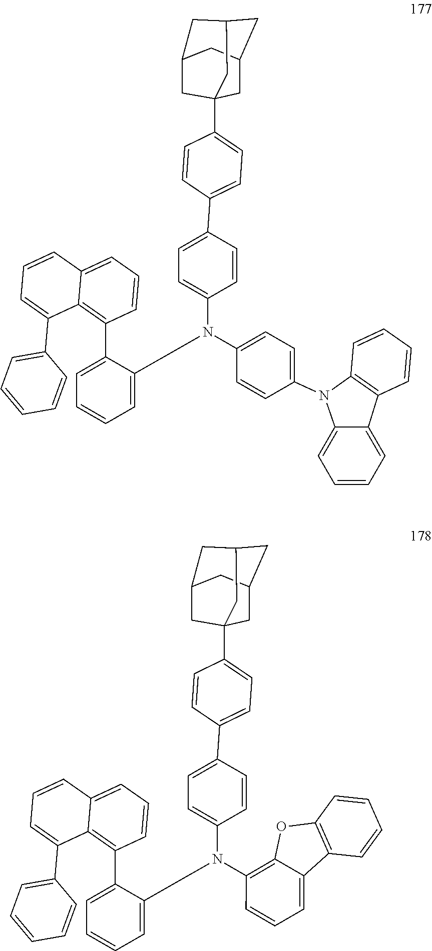 [SMILES: c1ccc(-c2cccc3cccc(-c4ccccc4N(c4ccc(-c5ccc(C67CC8CC(CC(C8)C6)C7)cc5)cc4)c4ccc(-n5c6ccccc6c6ccccc65)cc4)c23)cc1 ; c1ccc(-c2cccc3cccc(-c4ccccc4N(c4ccc(-c5ccc(C67CC8CC(CC(C8)C6)C7)cc5)cc4)c4cccc5c4oc4ccccc45)c23)cc1]

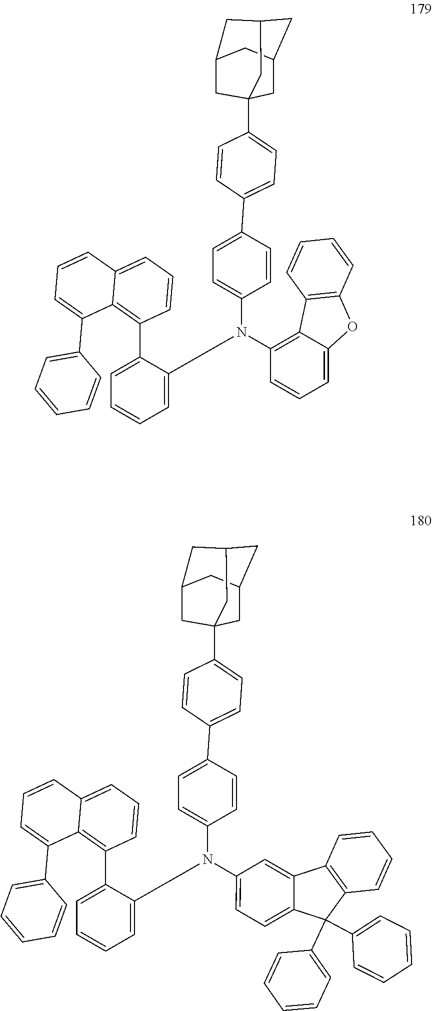 [SMILES: c1ccc(-c2cccc3cccc(-c4ccccc4N(c4ccc(-c5ccc(C67CC8CC(CC(C8)C6)C7)cc5)cc4)c4cccc5oc6ccccc6c45)c23)cc1 ; c1ccc(-c2cccc3cccc(-c4ccccc4N(c4ccc(-c5ccc(C67CC8CC(CC(C8)C6)C7)cc5)cc4)c4ccc5c(c4)-c4ccccc4C5(c4ccccc4)c4ccccc4)c23)cc1]

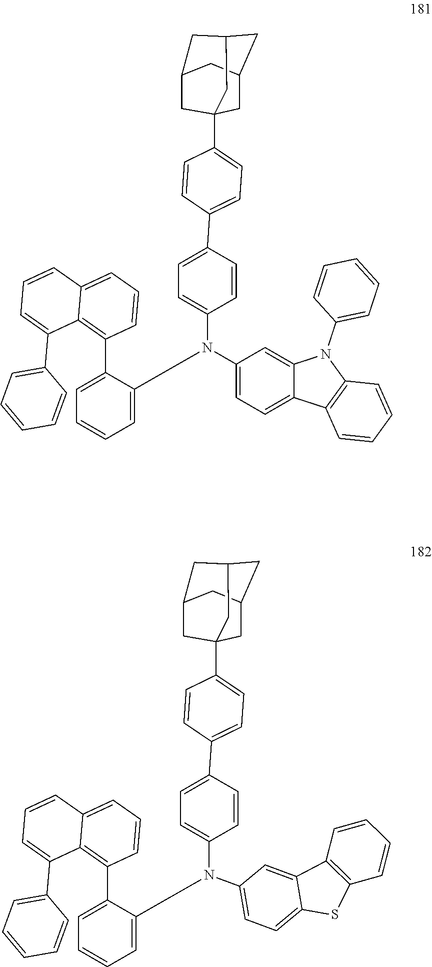 [SMILES: c1ccc(-c2cccc3cccc(-c4ccccc4N(c4ccc(-c5ccc(C67CC8CC(CC(C8)C6)C7)cc5)cc4)c4ccc5c6ccccc6n(-c6ccccc6)c5c4)c23)cc1 ; c1ccc(-c2cccc3cccc(-c4ccccc4N(c4ccc(-c5ccc(C67CC8CC(CC(C8)C6)C7)cc5)cc4)c4ccc5sc6ccccc6c5c4)c23)cc1]

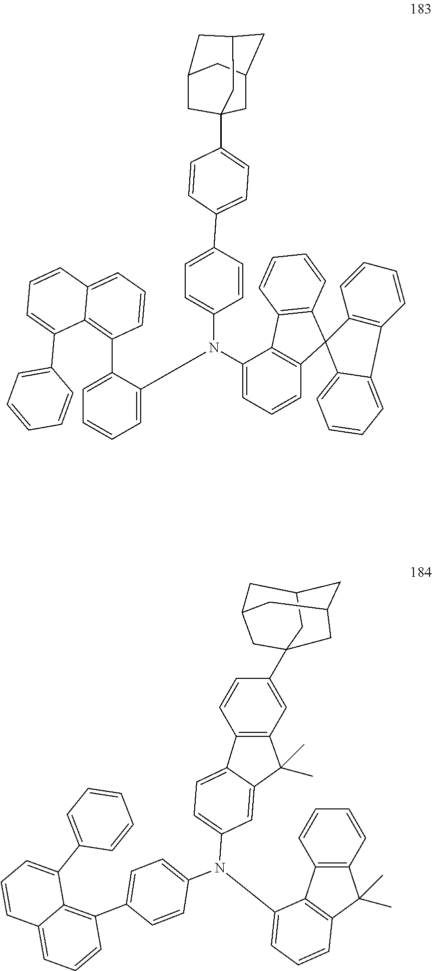 [SMILES: c1ccc(-c2cccc3cccc(-c4ccccc4N(c4ccc(-c5ccc(C67CC8CC(CC(C8)C6)C7)cc5)cc4)c4cccc5c4-c4ccccc4C54c5ccccc5-c5ccccc54)c23)cc1 ; CC1(C)c2cc(N(c3ccc(-c4cccc5cccc(-c6ccccc6)c45)cc3)c3cccc4c3-c3ccccc3C4(C)C)ccc2-c2ccc(C34CC5CC(CC(C5)C3)C4)cc21]

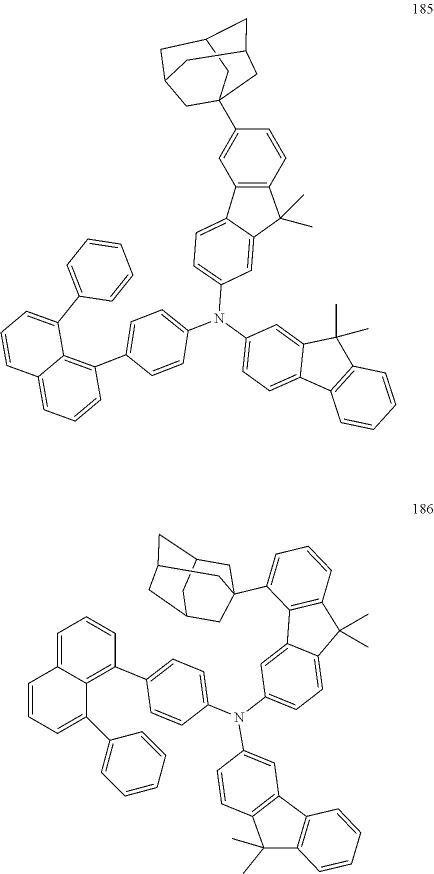 [SMILES: CC1(C)c2ccccc2-c2ccc(N(c3ccc(-c4cccc5cccc(-c6ccccc6)c45)cc3)c3ccc4c(c3)C(C)(C)c3ccc(C56CC7CC(CC(C7)C5)C6)cc3-4)cc21 ; CC1(C)c2ccccc2-c2cc(N(c3ccc(-c4cccc5cccc(-c6ccccc6)c45)cc3)c3ccc4c(c3)-c3c(C56CC7CC(CC(C7)C5)C6)cccc3C4(C)C)ccc21]

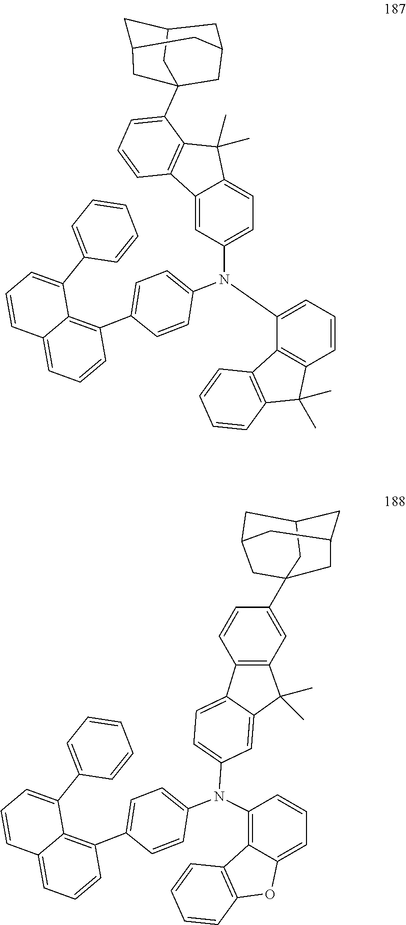 [SMILES: CC1(C)c2ccccc2-c2c(N(c3ccc(-c4cccc5cccc(-c6ccccc6)c45)cc3)c3ccc4c(c3)-c3cccc(C56CC7CC(CC(C7)C5)C6)c3C4(C)C)cccc21 ; CC1(C)c2cc(N(c3ccc(-c4cccc5cccc(-c6ccccc6)c45)cc3)c3cccc4oc5ccccc5c34)ccc2-c2ccc(C34CC5CC(CC(C5)C3)C4)cc21]

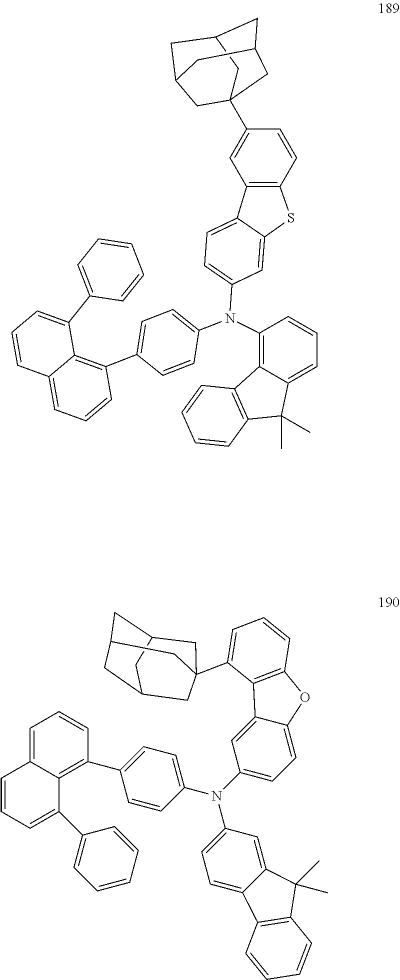 [SMILES: CC1(C)c2ccccc2-c2c(N(c3ccc(-c4cccc5cccc(-c6ccccc6)c45)cc3)c3ccc4c(c3)sc3ccc(C56CC7CC(CC(C7)C5)C6)cc34)cccc21 ; CC1(C)c2ccccc2-c2ccc(N(c3ccc(-c4cccc5cccc(-c6ccccc6)c45)cc3)c3ccc4oc5cccc(C67CC8CC(CC(C8)C6)C7)c5c4c3)cc21]

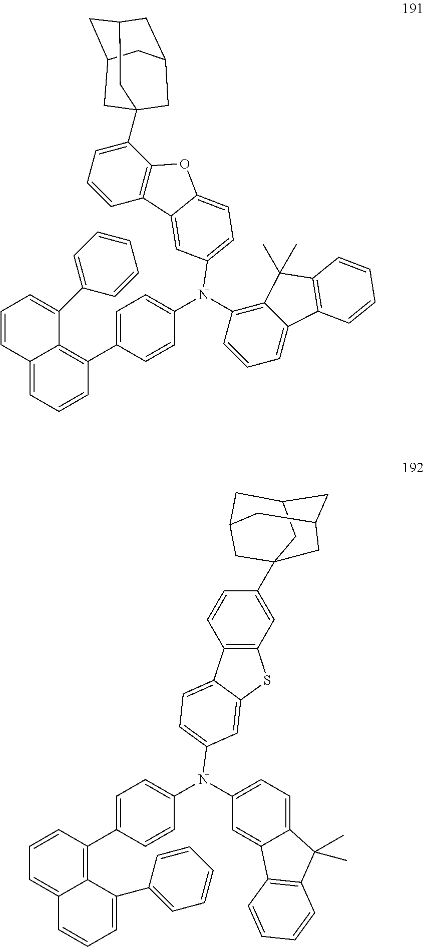 [SMILES: CC1(C)c2ccccc2-c2cccc(N(c3ccc(-c4cccc5cccc(-c6ccccc6)c45)cc3)c3ccc4oc5c(C67CC8CC(CC(C8)C6)C7)cccc5c4c3)c21 ; CC1(C)c2ccccc2-c2cc(N(c3ccc(-c4cccc5cccc(-c6ccccc6)c45)cc3)c3ccc4c(c3)sc3cc(C56CC7CC(CC(C7)C5)C6)ccc34)ccc21]

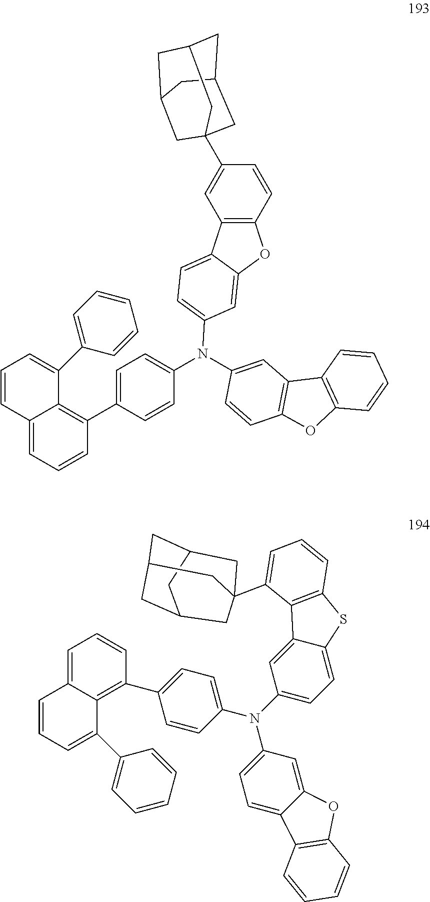 [SMILES: c1ccc(-c2cccc3cccc(-c4ccc(N(c5ccc6c(c5)oc5ccc(C78CC9CC(CC(C9)C7)C8)cc56)c5ccc6oc7ccccc7c6c5)cc4)c23)cc1 ; c1ccc(-c2cccc3cccc(-c4ccc(N(c5ccc6c(c5)oc5ccccc56)c5ccc6sc7cccc(C89CC%10CC(CC(C%10)C8)C9)c7c6c5)cc4)c23)cc1]

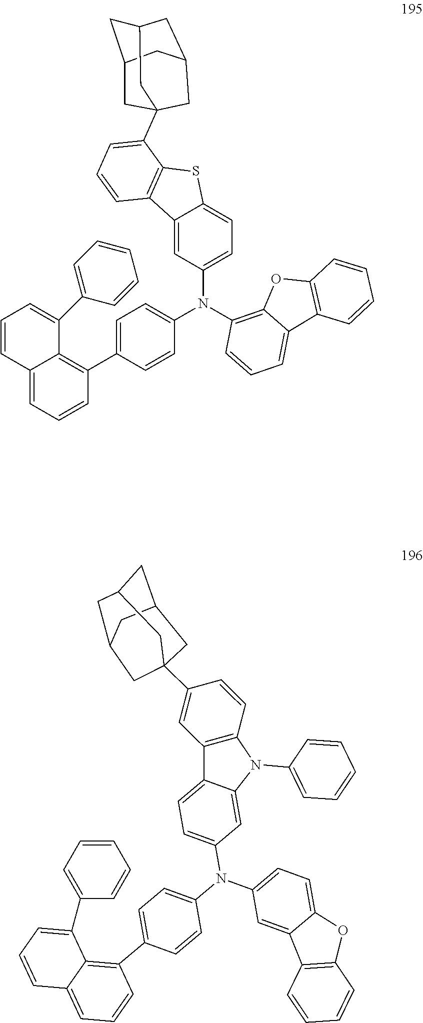 [SMILES: c1ccc(-c2cccc3cccc(-c4ccc(N(c5ccc6sc7c(C89CC%10CC(CC(C%10)C8)C9)cccc7c6c5)c5cccc6c5oc5ccccc56)cc4)c23)cc1 ; c1ccc(-c2cccc3cccc(-c4ccc(N(c5ccc6oc7ccccc7c6c5)c5ccc6c7cc(C89CC%10CC(CC(C%10)C8)C9)ccc7n(-c7ccccc7)c6c5)cc4)c23)cc1]

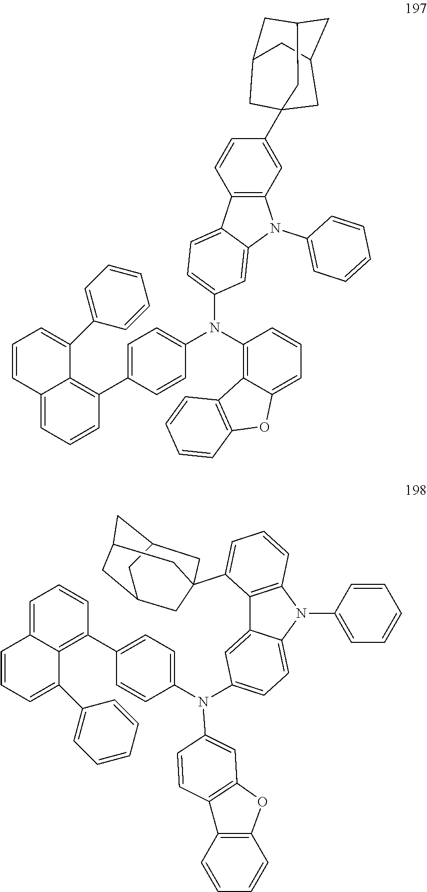 [SMILES: c1ccc(-c2cccc3cccc(-c4ccc(N(c5ccc6c7ccc(C89CC%10CC(CC(C%10)C8)C9)cc7n(-c7ccccc7)c6c5)c5cccc6oc7ccccc7c56)cc4)c23)cc1 ; c1ccc(-c2cccc3cccc(-c4ccc(N(c5ccc6c(c5)oc5ccccc56)c5ccc6c(c5)c5c(C78CC9CC(CC(C9)C7)C8)cccc5n6-c5ccccc5)cc4)c23)cc1]

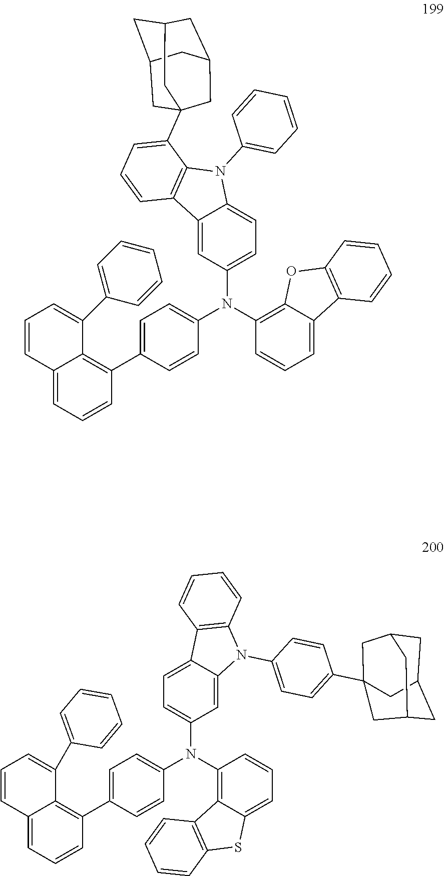 [SMILES: c1ccc(-c2cccc3cccc(-c4ccc(N(c5ccc6c(c5)c5cccc(C78CC9CC(CC(C9)C7)C8)c5n6-c5ccccc5)c5cccc6c5oc5ccccc56)cc4)c23)cc1 ; c1ccc(-c2cccc3cccc(-c4ccc(N(c5ccc6c7ccccc7n(-c7ccc(C89CC%10CC(CC(C%10)C8)C9)cc7)c6c5)c5cccc6sc7ccccc7c56)cc4)c23)cc1]

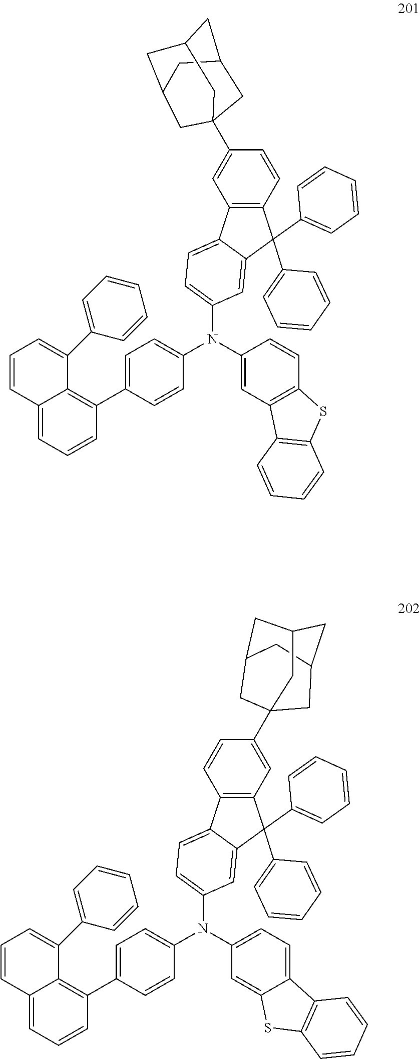 [SMILES: c1ccc(-c2cccc3cccc(-c4ccc(N(c5ccc6c(c5)C(c5ccccc5)(c5ccccc5)c5ccc(C78CC9CC(CC(C9)C7)C8)cc5-6)c5ccc6sc7ccccc7c6c5)cc4)c23)cc1 ; c1ccc(-c2cccc3cccc(-c4ccc(N(c5ccc6c(c5)C(c5ccccc5)(c5ccccc5)c5cc(C78CC9CC(CC(C9)C7)C8)ccc5-6)c5ccc6c(c5)sc5ccccc56)cc4)c23)cc1]

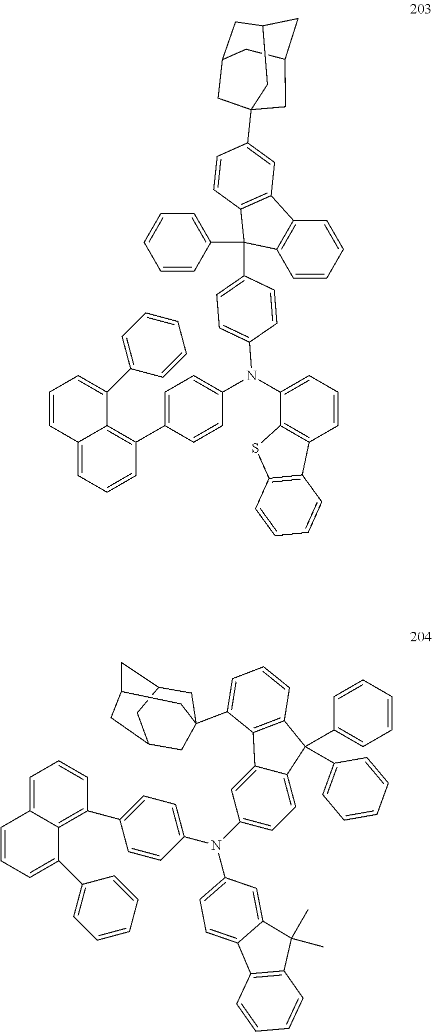 [SMILES: c1ccc(-c2cccc3cccc(-c4ccc(N(c5ccc(C6(c7ccccc7)c7ccccc7-c7cc(C89CC%10CC(CC(C%10)C8)C9)ccc76)cc5)c5cccc6c5sc5ccccc56)cc4)c23)cc1 ; CC1(C)c2ccccc2-c2ccc(N(c3ccc(-c4cccc5cccc(-c6ccccc6)c45)cc3)c3ccc4c(c3)-c3c(C56CC7CC(CC(C7)C5)C6)cccc3C4(c3ccccc3)c3ccccc3)cc21]

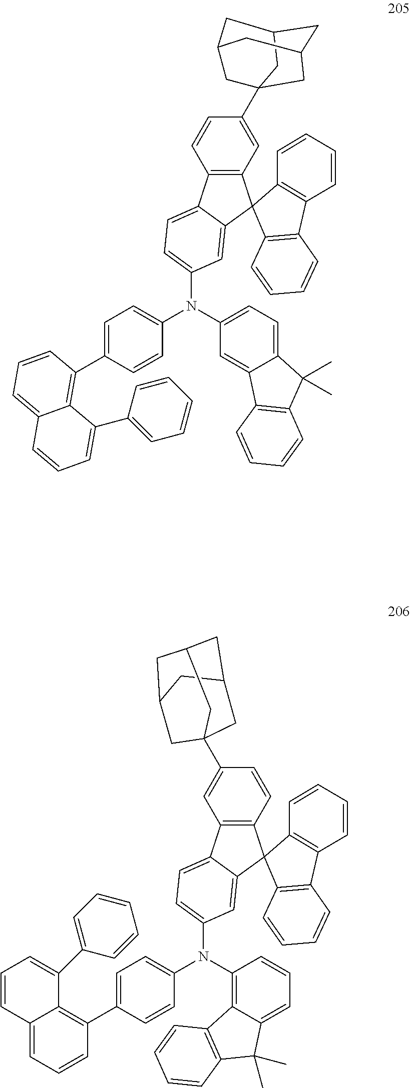 [SMILES: CC1(C)c2ccccc2-c2cc(N(c3ccc(-c4cccc5cccc(-c6ccccc6)c45)cc3)c3ccc4c(c3)C3(c5ccccc5-c5ccccc53)c3cc(C56CC7CC(CC(C7)C5)C6)ccc3-4)ccc21 ; CC1(C)c2ccccc2-c2c(N(c3ccc(-c4cccc5cccc(-c6ccccc6)c45)cc3)c3ccc4c(c3)C3(c5ccccc5-c5ccccc53)c3ccc(C56CC7CC(CC(C7)C5)C6)cc3-4)cccc21]

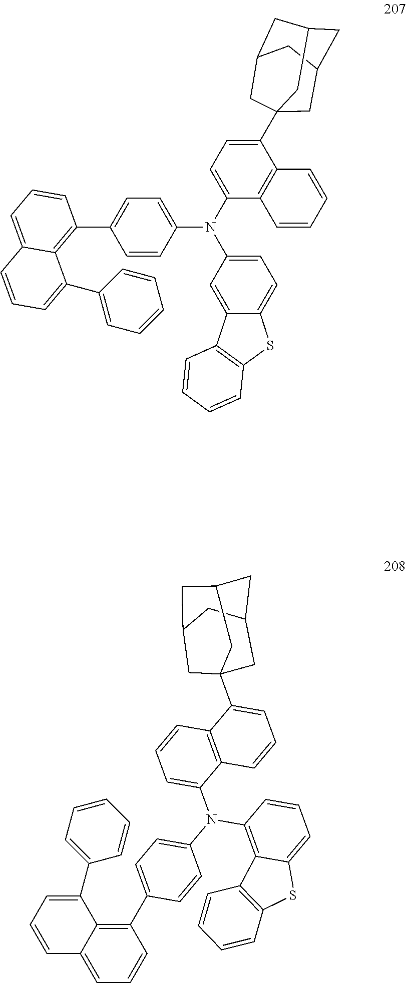 [SMILES: c1ccc(-c2cccc3cccc(-c4ccc(N(c5ccc6sc7ccccc7c6c5)c5ccc(C67CC8CC(CC(C8)C6)C7)c6ccccc56)cc4)c23)cc1 ; c1ccc(-c2cccc3cccc(-c4ccc(N(c5cccc6c(C78CC9CC(CC(C9)C7)C8)cccc56)c5cccc6sc7ccccc7c56)cc4)c23)cc1]

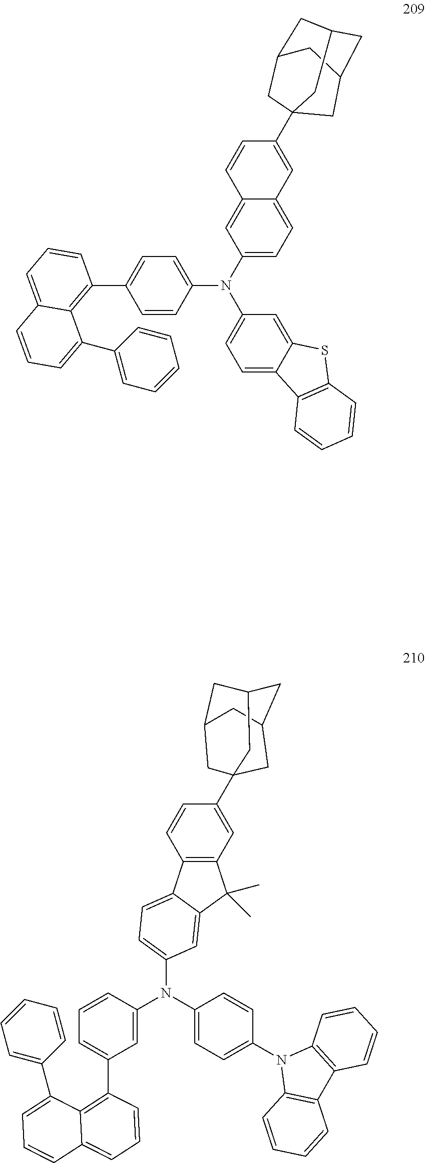 [SMILES: c1ccc(-c2cccc3cccc(-c4ccc(N(c5ccc6cc(C78CC9CC(CC(C9)C7)C8)ccc6c5)c5ccc6c(c5)sc5ccccc56)cc4)c23)cc1 ; CC1(C)c2cc(N(c3ccc(-n4c5ccccc5c5ccccc54)cc3)c3cccc(-c4cccc5cccc(-c6ccccc6)c45)c3)ccc2-c2ccc(C34CC5CC(CC(C5)C3)C4)cc21]

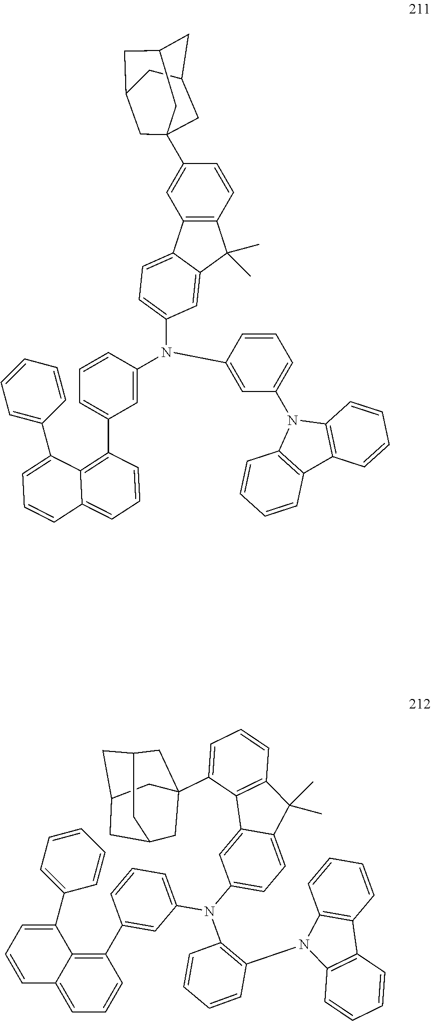 [SMILES: CC1(C)c2ccc(C34CC5CC(CC(C5)C3)C4)cc2-c2ccc(N(c3cccc(-c4cccc5cccc(-c6ccccc6)c45)c3)c3cccc(-n4c5ccccc5c5ccccc54)c3)cc21 ; CC1(C)c2ccc(N(c3cccc(-c4cccc5cccc(-c6ccccc6)c45)c3)c3ccccc3-n3c4ccccc4c4ccccc43)cc2-c2c(C34CC5CC(CC(C5)C3)C4)cccc21]

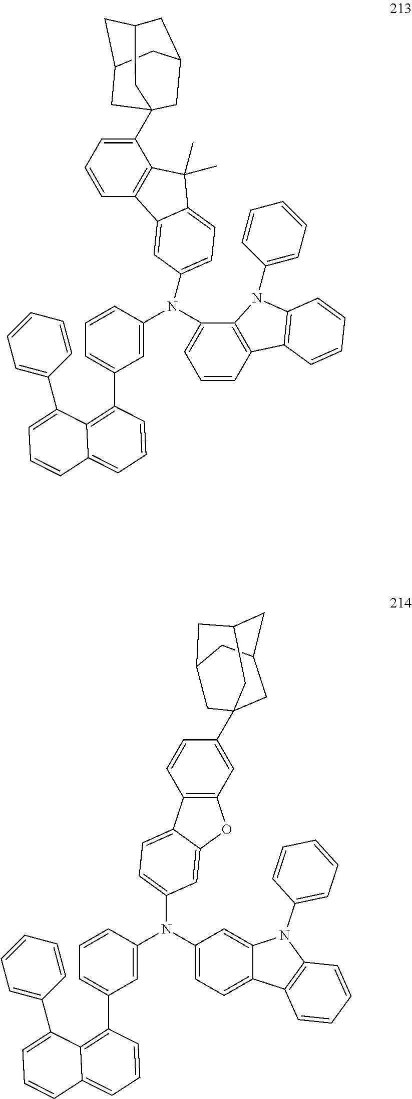 [SMILES: CC1(C)c2ccc(N(c3cccc(-c4cccc5cccc(-c6ccccc6)c45)c3)c3cccc4c5ccccc5n(-c5ccccc5)c34)cc2-c2cccc(C34CC5CC(CC(C5)C3)C4)c21 ; c1ccc(-c2cccc3cccc(-c4cccc(N(c5ccc6c(c5)oc5cc(C78CC9CC(CC(C9)C7)C8)ccc56)c5ccc6c7ccccc7n(-c7ccccc7)c6c5)c4)c23)cc1]

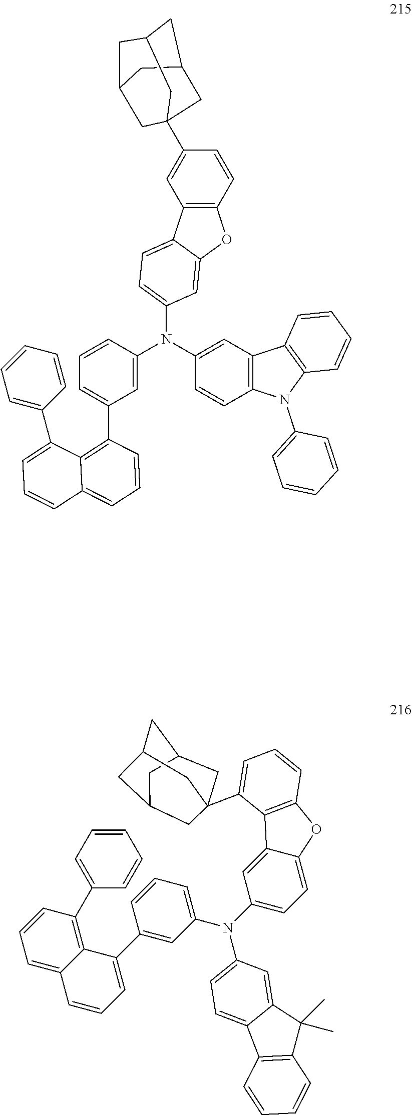 [SMILES: c1ccc(-c2cccc3cccc(-c4cccc(N(c5ccc6c(c5)oc5ccc(C78CC9CC(CC(C9)C7)C8)cc56)c5ccc6c(c5)c5ccccc5n6-c5ccccc5)c4)c23)cc1 ; CC1(C)c2ccccc2-c2ccc(N(c3cccc(-c4cccc5cccc(-c6ccccc6)c45)c3)c3ccc4oc5cccc(C67CC8CC(CC(C8)C6)C7)c5c4c3)cc21]

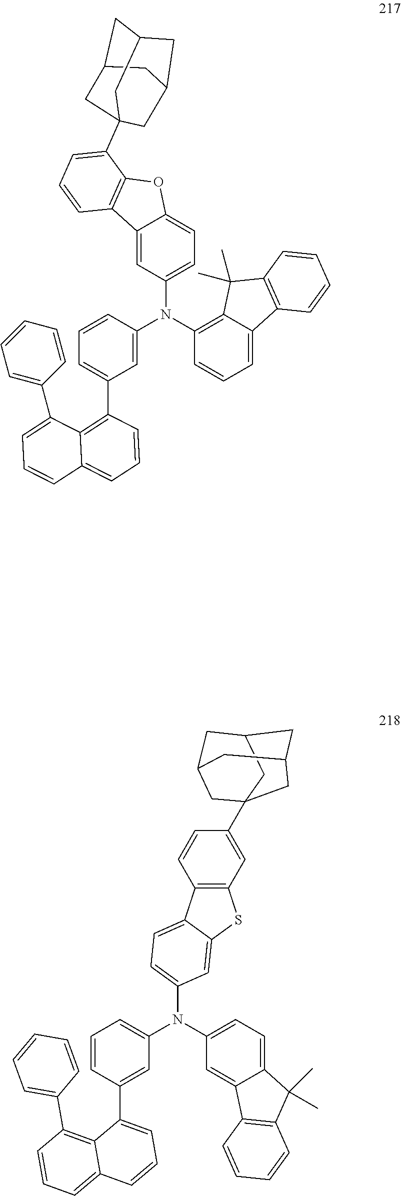 [SMILES: CC1(C)c2ccccc2-c2cccc(N(c3cccc(-c4cccc5cccc(-c6ccccc6)c45)c3)c3ccc4oc5c(C67CC8CC(CC(C8)C6)C7)cccc5c4c3)c21 ; CC1(C)c2ccccc2-c2cc(N(c3cccc(-c4cccc5cccc(-c6ccccc6)c45)c3)c3ccc4c(c3)sc3cc(C56CC7CC(CC(C7)C5)C6)ccc34)ccc21]

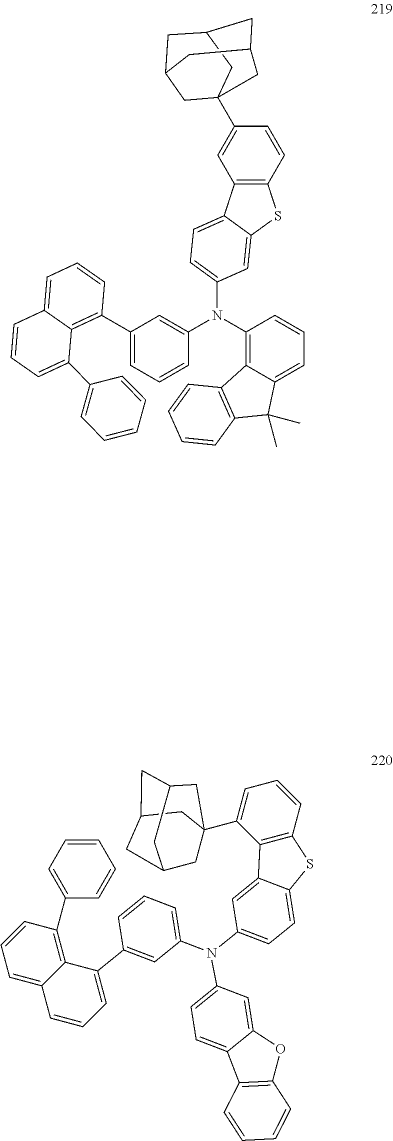 [SMILES: CC1(C)c2ccccc2-c2c(N(c3cccc(-c4cccc5cccc(-c6ccccc6)c45)c3)c3ccc4c(c3)sc3ccc(C56CC7CC(CC(C7)C5)C6)cc34)cccc21 ; c1ccc(-c2cccc3cccc(-c4cccc(N(c5ccc6c(c5)oc5ccccc56)c5ccc6sc7cccc(C89CC%10CC(CC(C%10)C8)C9)c7c6c5)c4)c23)cc1]

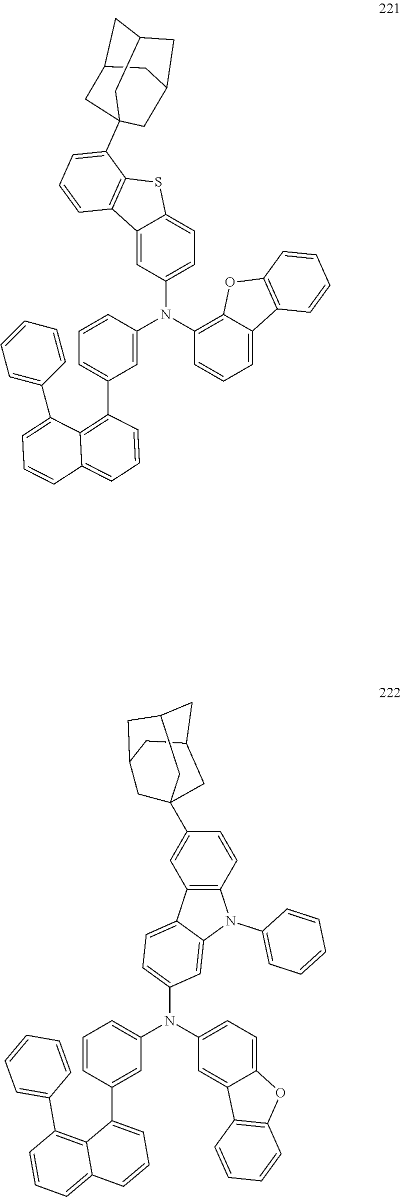 [SMILES: c1ccc(-c2cccc3cccc(-c4cccc(N(c5ccc6sc7c(C89CC%10CC(CC(C%10)C8)C9)cccc7c6c5)c5cccc6c5oc5ccccc56)c4)c23)cc1 ; c1ccc(-c2cccc3cccc(-c4cccc(N(c5ccc6oc7ccccc7c6c5)c5ccc6c7cc(C89CC%10CC(CC(C%10)C8)C9)ccc7n(-c7ccccc7)c6c5)c4)c23)cc1]

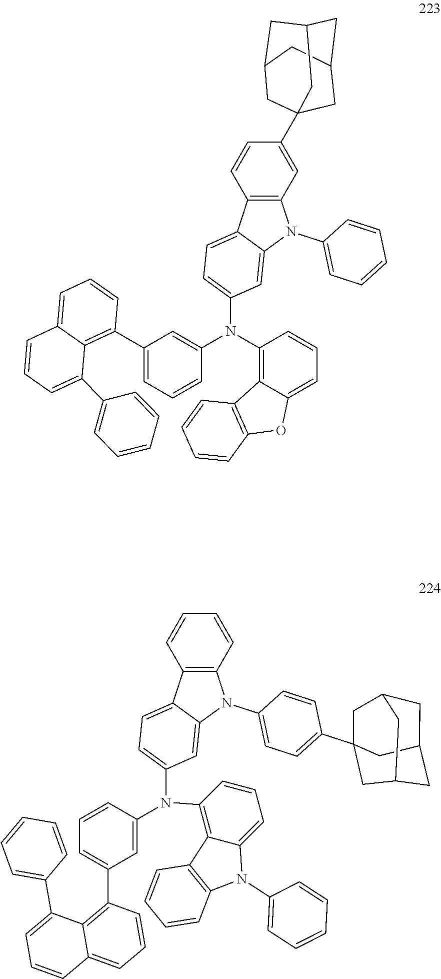 [SMILES: c1ccc(-c2cccc3cccc(-c4cccc(N(c5ccc6c7ccc(C89CC%10CC(CC(C%10)C8)C9)cc7n(-c7ccccc7)c6c5)c5cccc6oc7ccccc7c56)c4)c23)cc1 ; c1ccc(-c2cccc3cccc(-c4cccc(N(c5ccc6c7ccccc7n(-c7ccc(C89CC%10CC(CC(C%10)C8)C9)cc7)c6c5)c5cccc6c5c5ccccc5n6-c5ccccc5)c4)c23)cc1]

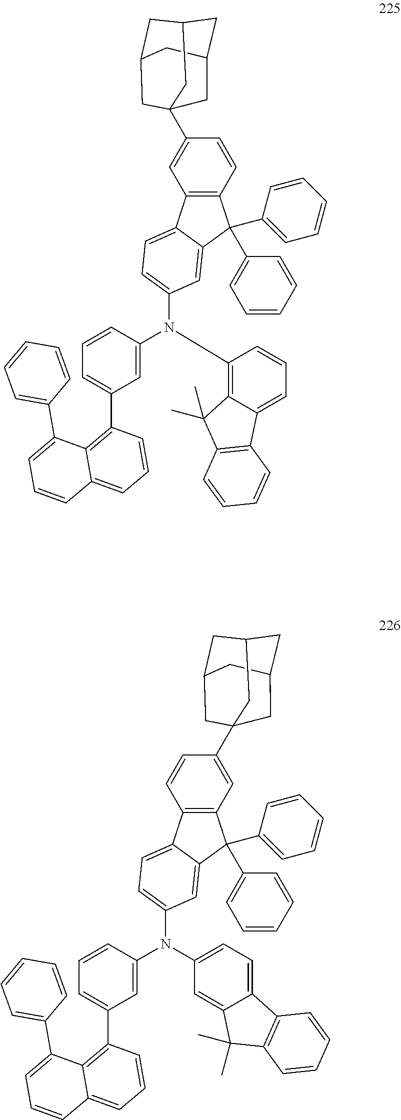 [SMILES: CC1(C)c2ccccc2-c2cccc(N(c3cccc(-c4cccc5cccc(-c6ccccc6)c45)c3)c3ccc4c(c3)C(c3ccccc3)(c3ccccc3)c3ccc(C56CC7CC(CC(C7)C5)C6)cc3-4)c21 ; CC1(C)c2ccccc2-c2ccc(N(c3cccc(-c4cccc5cccc(-c6ccccc6)c45)c3)c3ccc4c(c3)C(c3ccccc3)(c3ccccc3)c3cc(C56CC7CC(CC(C7)C5)C6)ccc3-4)cc21]

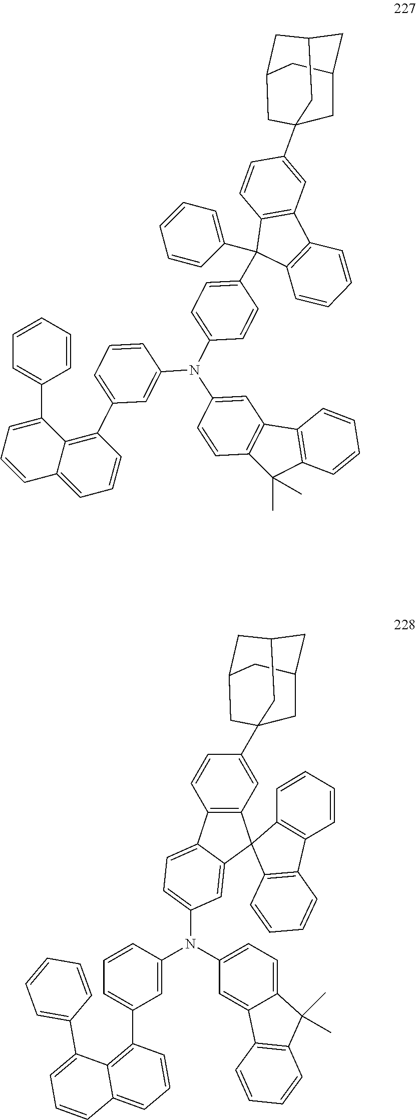 [SMILES: CC1(C)c2ccccc2-c2cc(N(c3ccc(C4(c5ccccc5)c5ccccc5-c5cc(C67CC8CC(CC(C8)C6)C7)ccc54)cc3)c3cccc(-c4cccc5cccc(-c6ccccc6)c45)c3)ccc21 ; CC1(C)c2ccccc2-c2cc(N(c3cccc(-c4cccc5cccc(-c6ccccc6)c45)c3)c3ccc4c(c3)C3(c5ccccc5-c5ccccc53)c3cc(C56CC7CC(CC(C7)C5)C6)ccc3-4)ccc21]

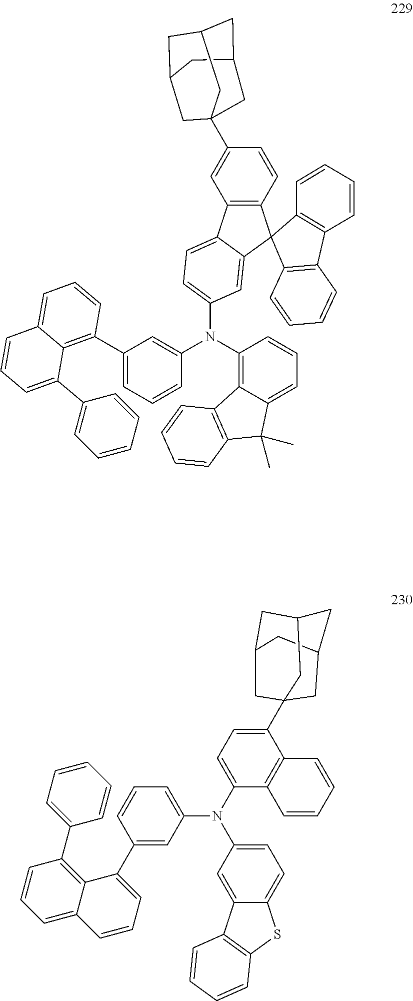 [SMILES: CC1(C)c2ccccc2-c2c(N(c3cccc(-c4cccc5cccc(-c6ccccc6)c45)c3)c3ccc4c(c3)C3(c5ccccc5-c5ccccc53)c3ccc(C56CC7CC(CC(C7)C5)C6)cc3-4)cccc21 ; c1ccc(-c2cccc3cccc(-c4cccc(N(c5ccc6sc7ccccc7c6c5)c5ccc(C67CC8CC(CC(C8)C6)C7)c6ccccc56)c4)c23)cc1]

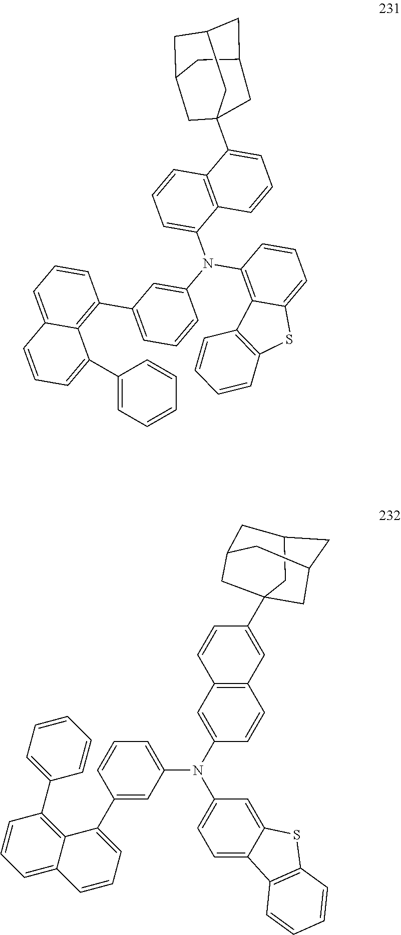 [SMILES: c1ccc(-c2cccc3cccc(-c4cccc(N(c5cccc6c(C78CC9CC(CC(C9)C7)C8)cccc56)c5cccc6sc7ccccc7c56)c4)c23)cc1 ; c1ccc(-c2cccc3cccc(-c4cccc(N(c5ccc6cc(C78CC9CC(CC(C9)C7)C8)ccc6c5)c5ccc6c(c5)sc5ccccc56)c4)c23)cc1]

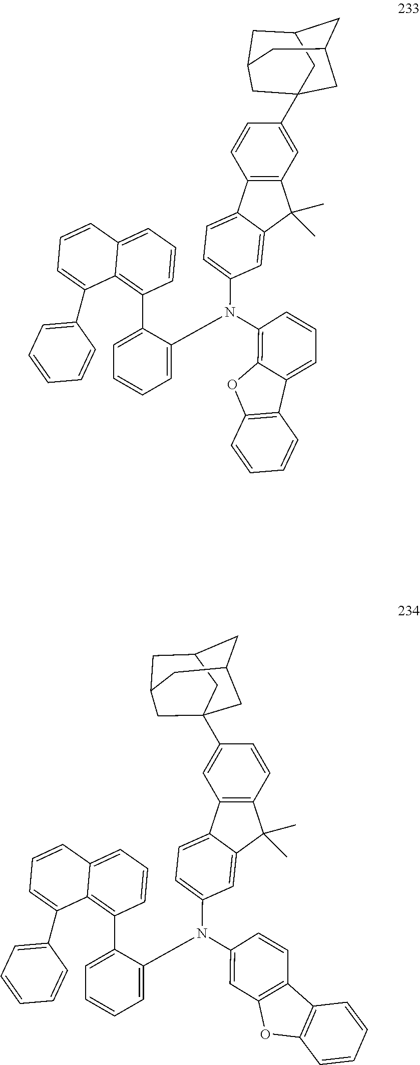 [SMILES: CC1(C)c2cc(N(c3ccccc3-c3cccc4cccc(-c5ccccc5)c34)c3cccc4c3oc3ccccc34)ccc2-c2ccc(C34CC5CC(CC(C5)C3)C4)cc21 ; CC1(C)c2ccc(C34CC5CC(CC(C5)C3)C4)cc2-c2ccc(N(c3ccc4c(c3)oc3ccccc34)c3ccccc3-c3cccc4cccc(-c5ccccc5)c34)cc21]

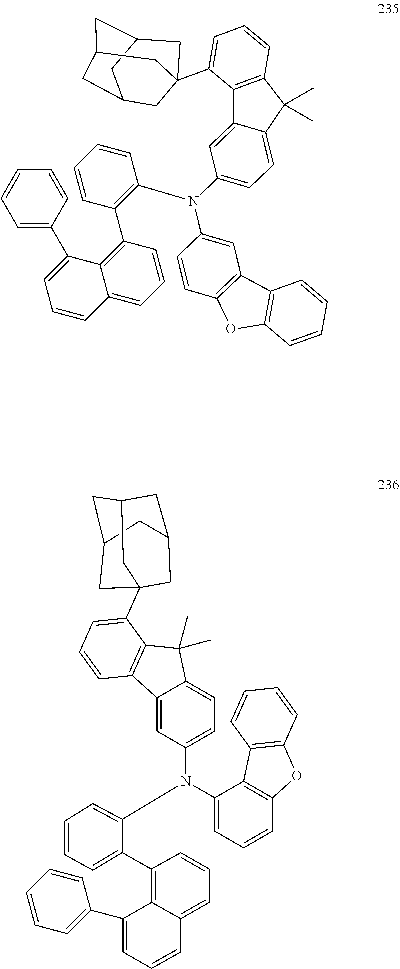 [SMILES: CC1(C)c2ccc(N(c3ccc4oc5ccccc5c4c3)c3ccccc3-c3cccc4cccc(-c5ccccc5)c34)cc2-c2c(C34CC5CC(CC(C5)C3)C4)cccc21 ; CC1(C)c2ccc(N(c3ccccc3-c3cccc4cccc(-c5ccccc5)c34)c3cccc4oc5ccccc5c34)cc2-c2cccc(C34CC5CC(CC(C5)C3)C4)c21]

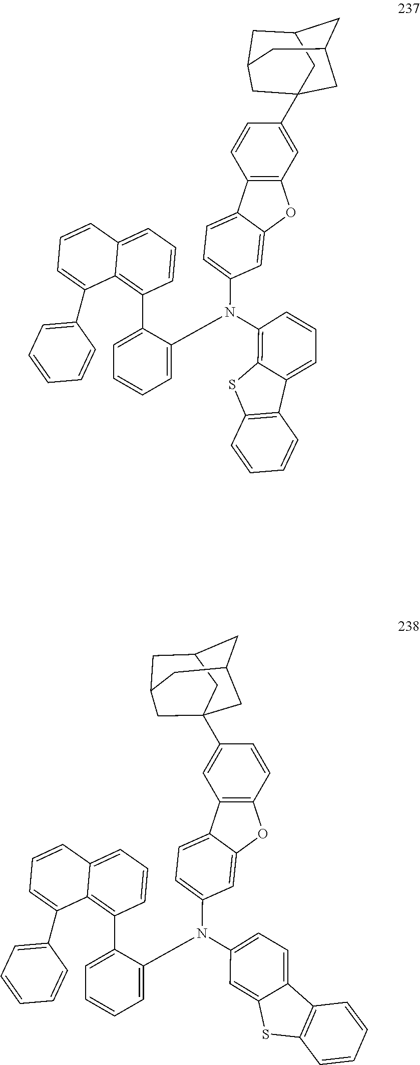 [SMILES: c1ccc(-c2cccc3cccc(-c4ccccc4N(c4ccc5c(c4)oc4cc(C67CC8CC(CC(C8)C6)C7)ccc45)c4cccc5c4sc4ccccc45)c23)cc1 ; c1ccc(-c2cccc3cccc(-c4ccccc4N(c4ccc5c(c4)oc4ccc(C67CC8CC(CC(C8)C6)C7)cc45)c4ccc5c(c4)sc4ccccc45)c23)cc1]

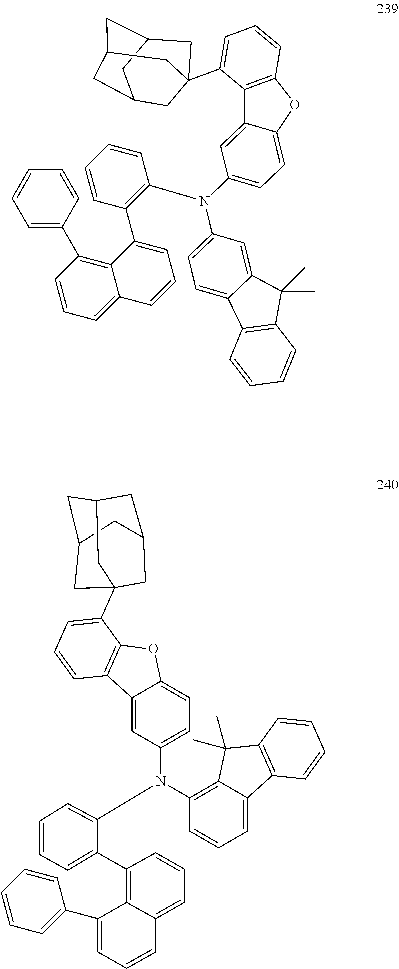 [SMILES: CC1(C)c2ccccc2-c2ccc(N(c3ccc4oc5cccc(C67CC8CC(CC(C8)C6)C7)c5c4c3)c3ccccc3-c3cccc4cccc(-c5ccccc5)c34)cc21 ; CC1(C)c2ccccc2-c2cccc(N(c3ccc4oc5c(C67CC8CC(CC(C8)C6)C7)cccc5c4c3)c3ccccc3-c3cccc4cccc(-c5ccccc5)c34)c21]

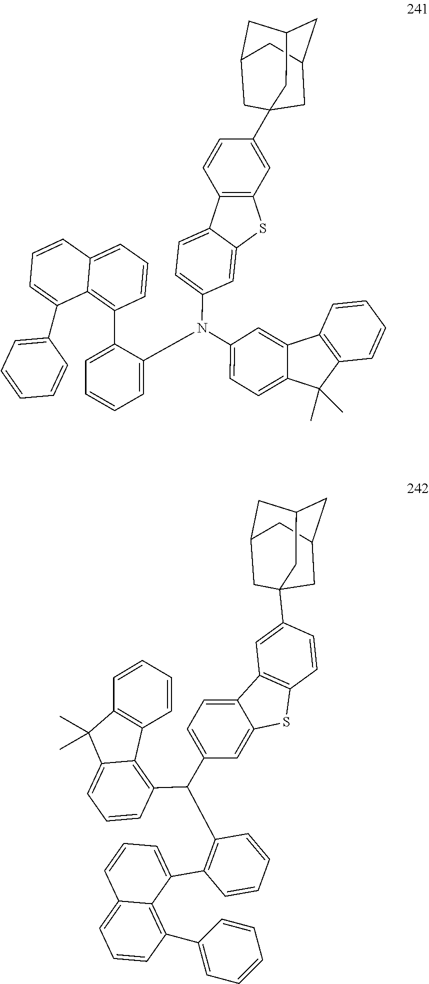 [SMILES: CC1(C)c2ccccc2-c2cc(N(c3ccc4c(c3)sc3cc(C56CC7CC(CC(C7)C5)C6)ccc34)c3ccccc3-c3cccc4cccc(-c5ccccc5)c34)ccc21 ; CC1(C)c2ccccc2-c2c(C(c3ccc4c(c3)sc3ccc(C56CC7CC(CC(C7)C5)C6)cc34)c3ccccc3-c3cccc4cccc(-c5ccccc5)c34)cccc21]

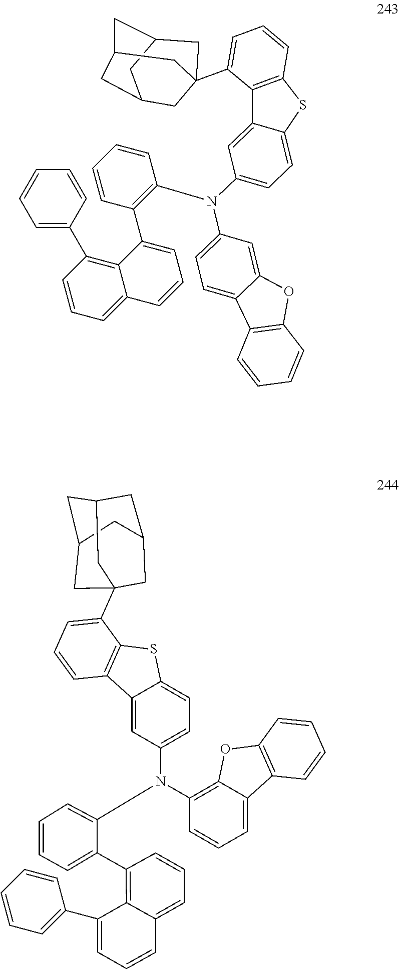 [SMILES: c1ccc(-c2cccc3cccc(-c4ccccc4N(c4ccc5c(c4)oc4ccccc45)c4ccc5sc6cccc(C78CC9CC(CC(C9)C7)C8)c6c5c4)c23)cc1 ; c1ccc(-c2cccc3cccc(-c4ccccc4N(c4ccc5sc6c(C78CC9CC(CC(C9)C7)C8)cccc6c5c4)c4cccc5c4oc4ccccc45)c23)cc1]

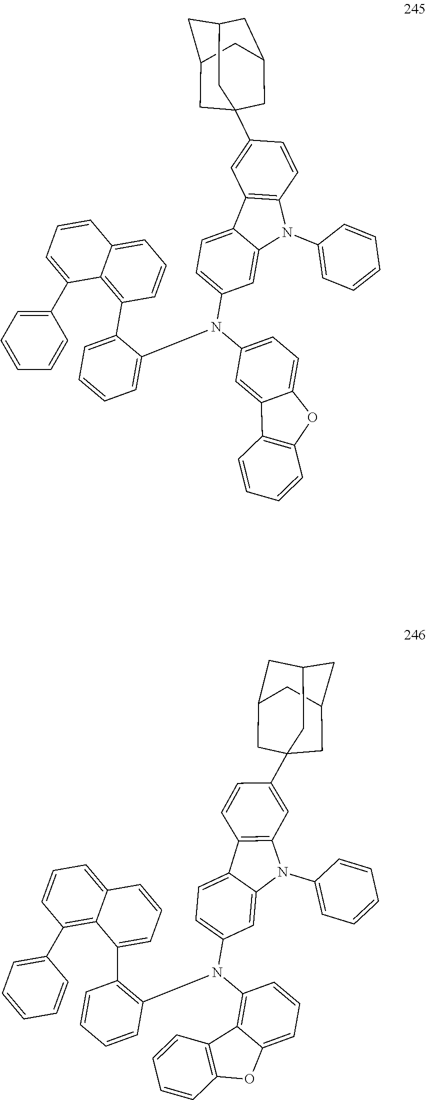 [SMILES: c1ccc(-c2cccc3cccc(-c4ccccc4N(c4ccc5oc6ccccc6c5c4)c4ccc5c6cc(C78CC9CC(CC(C9)C7)C8)ccc6n(-c6ccccc6)c5c4)c23)cc1 ; c1ccc(-c2cccc3cccc(-c4ccccc4N(c4ccc5c6ccc(C78CC9CC(CC(C9)C7)C8)cc6n(-c6ccccc6)c5c4)c4cccc5oc6ccccc6c45)c23)cc1]

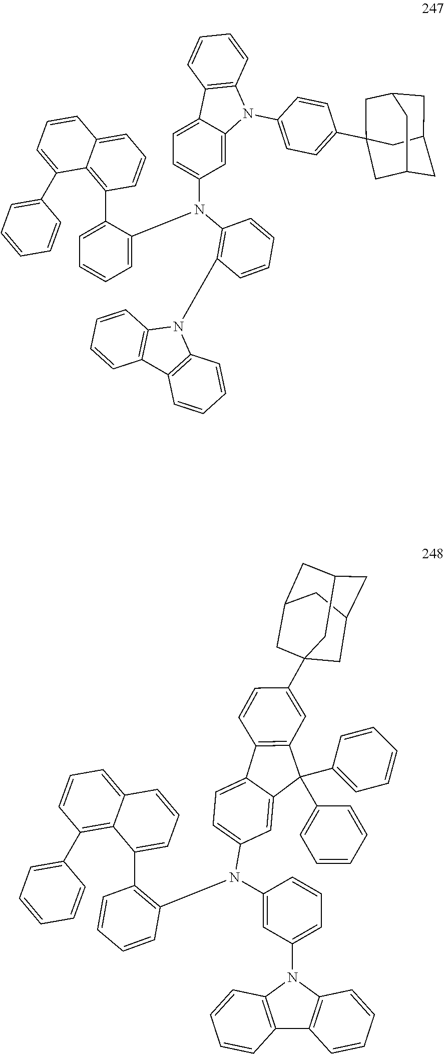 [SMILES: c1ccc(-c2cccc3cccc(-c4ccccc4N(c4ccc5c6ccccc6n(-c6ccc(C78CC9CC(CC(C9)C7)C8)cc6)c5c4)c4ccccc4-n4c5ccccc5c5ccccc54)c23)cc1 ; c1ccc(-c2cccc3cccc(-c4ccccc4N(c4cccc(-n5c6ccccc6c6ccccc65)c4)c4ccc5c(c4)C(c4ccccc4)(c4ccccc4)c4cc(C67CC8CC(CC(C8)C6)C7)ccc4-5)c23)cc1]

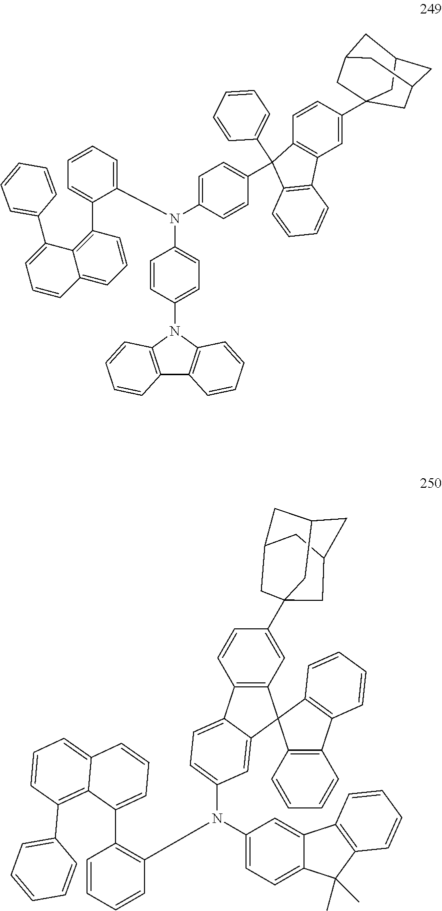 [SMILES: c1ccc(-c2cccc3cccc(-c4ccccc4N(c4ccc(-n5c6ccccc6c6ccccc65)cc4)c4ccc(C5(c6ccccc6)c6ccccc6-c6cc(C78CC9CC(CC(C9)C7)C8)ccc65)cc4)c23)cc1 ; CC1(C)c2ccccc2-c2cc(N(c3ccc4c(c3)C3(c5ccccc5-c5ccccc53)c3cc(C56CC7CC(CC(C7)C5)C6)ccc3-4)c3ccccc3-c3cccc4cccc(-c5ccccc5)c34)ccc21]

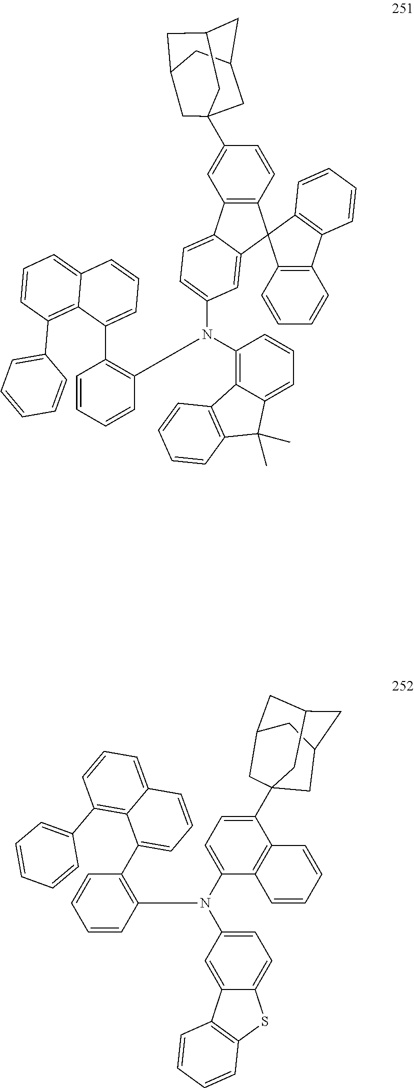 [SMILES: CC1(C)c2ccccc2-c2c(N(c3ccc4c(c3)C3(c5ccccc5-c5ccccc53)c3ccc(C56CC7CC(CC(C7)C5)C6)cc3-4)c3ccccc3-c3cccc4cccc(-c5ccccc5)c34)cccc21 ; c1ccc(-c2cccc3cccc(-c4ccccc4N(c4ccc5sc6ccccc6c5c4)c4ccc(C56CC7CC(CC(C7)C5)C6)c5ccccc45)c23)cc1]

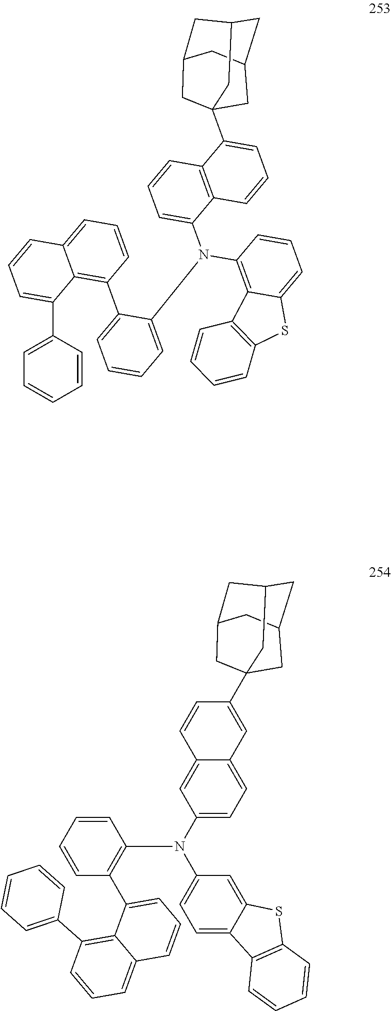 [SMILES: c1ccc(-c2cccc3cccc(-c4ccccc4N(c4cccc5c(C67CC8CC(CC(C8)C6)C7)cccc45)c4cccc5sc6ccccc6c45)c23)cc1 ; c1ccc(-c2cccc3cccc(-c4ccccc4N(c4ccc5cc(C67CC8CC(CC(C8)C6)C7)ccc5c4)c4ccc5c(c4)sc4ccccc45)c23)cc1]

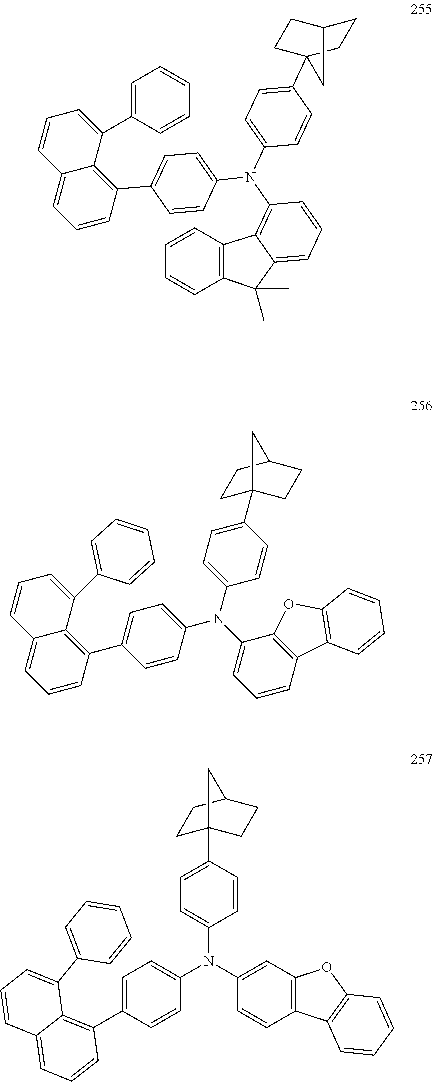 [SMILES: CC1(C)c2ccccc2-c2c(N(c3ccc(-c4cccc5cccc(-c6ccccc6)c45)cc3)c3ccc(C45CCC(CC4)C5)cc3)cccc21 ; c1ccc(-c2cccc3cccc(-c4ccc(N(c5ccc(C67CCC(CC6)C7)cc5)c5cccc6c5oc5ccccc56)cc4)c23)cc1 ; c1ccc(-c2cccc3cccc(-c4ccc(N(c5ccc(C67CCC(CC6)C7)cc5)c5ccc6c(c5)oc5ccccc56)cc4)c23)cc1]

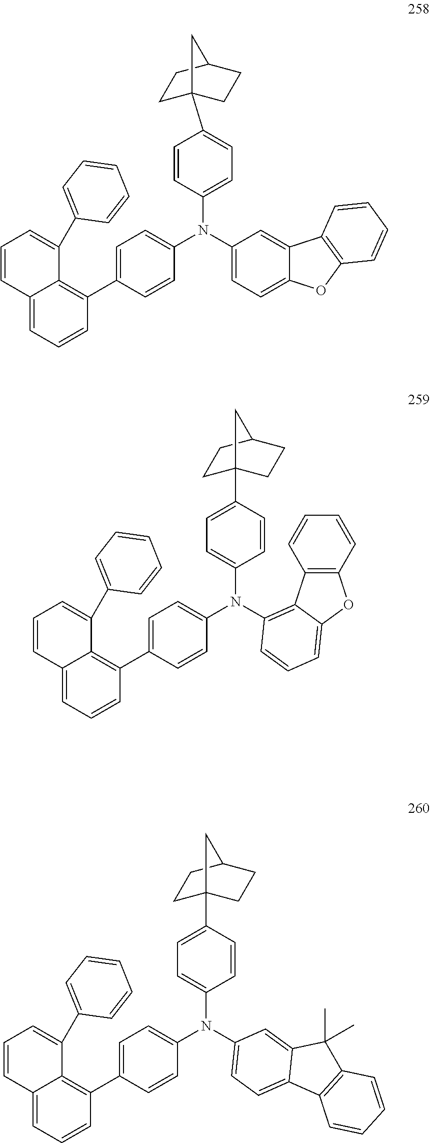 [SMILES: c1ccc(-c2cccc3cccc(-c4ccc(N(c5ccc(C67CCC(CC6)C7)cc5)c5ccc6oc7ccccc7c6c5)cc4)c23)cc1 ; c1ccc(-c2cccc3cccc(-c4ccc(N(c5ccc(C67CCC(CC6)C7)cc5)c5cccc6oc7ccccc7c56)cc4)c23)cc1 ; CC1(C)c2ccccc2-c2ccc(N(c3ccc(-c4cccc5cccc(-c6ccccc6)c45)cc3)c3ccc(C45CCC(CC4)C5)cc3)cc21]

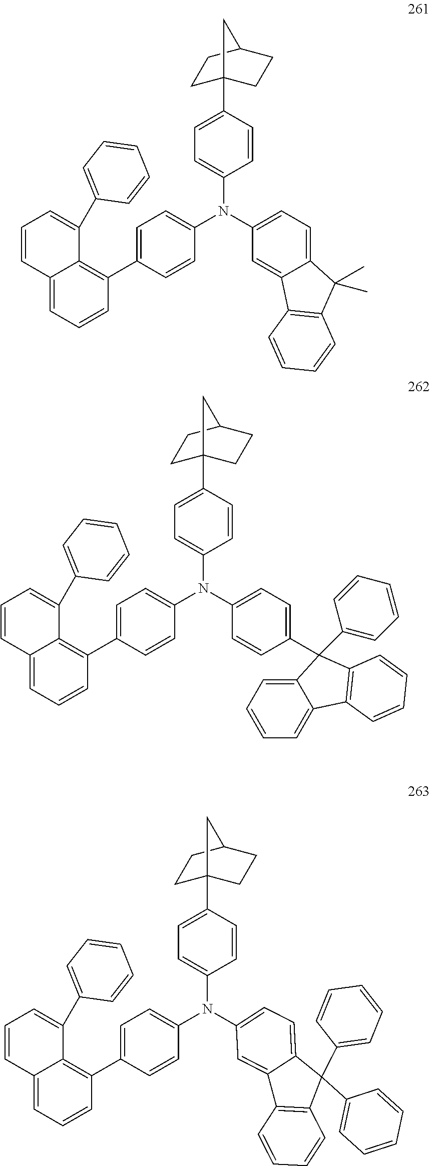 [SMILES: CC1(C)c2ccccc2-c2cc(N(c3ccc(-c4cccc5cccc(-c6ccccc6)c45)cc3)c3ccc(C45CCC(CC4)C5)cc3)ccc21 ; c1ccc(-c2cccc3cccc(-c4ccc(N(c5ccc(C67CCC(CC6)C7)cc5)c5ccc(C6(c7ccccc7)c7ccccc7-c7ccccc76)cc5)cc4)c23)cc1 ; c1ccc(-c2cccc3cccc(-c4ccc(N(c5ccc(C67CCC(CC6)C7)cc5)c5ccc6c(c5)-c5ccccc5C6(c5ccccc5)c5ccccc5)cc4)c23)cc1]

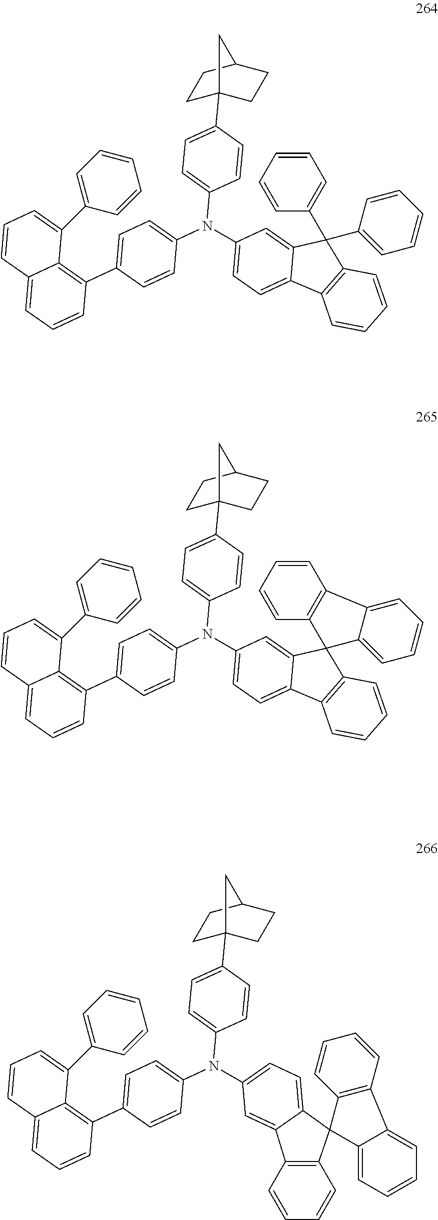 [SMILES: c1ccc(-c2cccc3cccc(-c4ccc(N(c5ccc(C67CCC(CC6)C7)cc5)c5ccc6c(c5)C(c5ccccc5)(c5ccccc5)c5ccccc5-6)cc4)c23)cc1 ; c1ccc(-c2cccc3cccc(-c4ccc(N(c5ccc(C67CCC(CC6)C7)cc5)c5ccc6c(c5)C5(c7ccccc7-c7ccccc75)c5ccccc5-6)cc4)c23)cc1 ; c1ccc(-c2cccc3cccc(-c4ccc(N(c5ccc(C67CCC(CC6)C7)cc5)c5ccc6c(c5)-c5ccccc5C65c6ccccc6-c6ccccc65)cc4)c23)cc1]

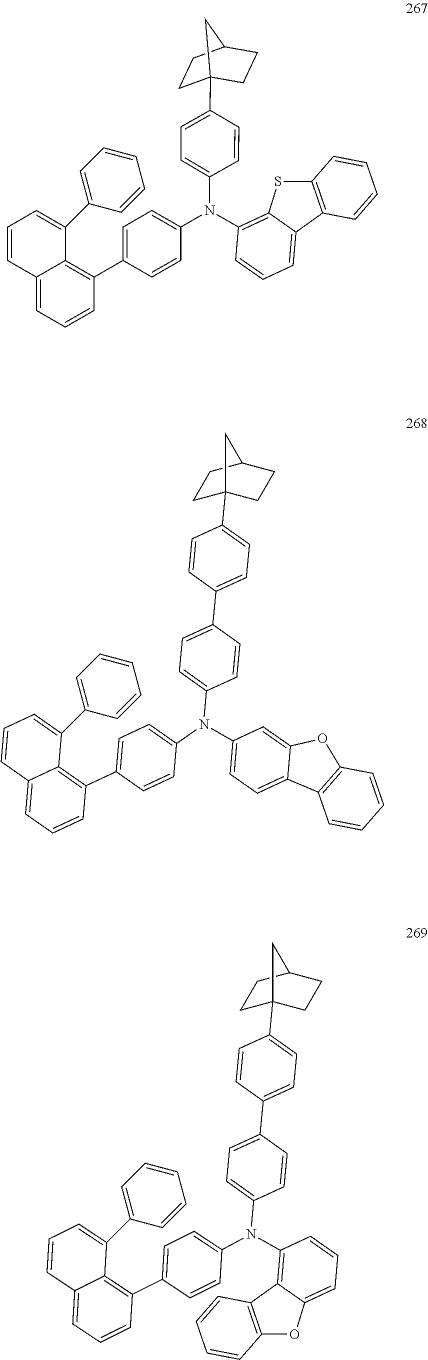 [SMILES: c1ccc(-c2cccc3cccc(-c4ccc(N(c5ccc(C67CCC(CC6)C7)cc5)c5cccc6c5sc5ccccc56)cc4)c23)cc1 ; c1ccc(-c2cccc3cccc(-c4ccc(N(c5ccc(-c6ccc(C78CCC(CC7)C8)cc6)cc5)c5ccc6c(c5)oc5ccccc56)cc4)c23)cc1 ; c1ccc(-c2cccc3cccc(-c4ccc(N(c5ccc(-c6ccc(C78CCC(CC7)C8)cc6)cc5)c5cccc6oc7ccccc7c56)cc4)c23)cc1]

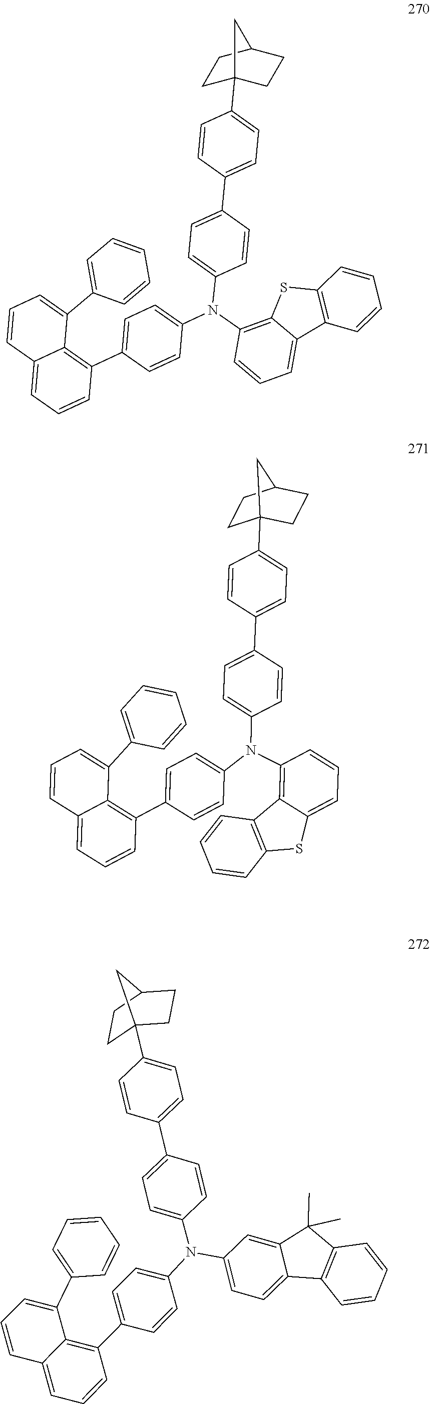 [SMILES: c1ccc(-c2cccc3cccc(-c4ccc(N(c5ccc(-c6ccc(C78CCC(CC7)C8)cc6)cc5)c5cccc6c5sc5ccccc56)cc4)c23)cc1 ; c1ccc(-c2cccc3cccc(-c4ccc(N(c5ccc(-c6ccc(C78CCC(CC7)C8)cc6)cc5)c5cccc6sc7ccccc7c56)cc4)c23)cc1 ; CC1(C)c2ccccc2-c2ccc(N(c3ccc(-c4ccc(C56CCC(CC5)C6)cc4)cc3)c3ccc(-c4cccc5cccc(-c6ccccc6)c45)cc3)cc21]

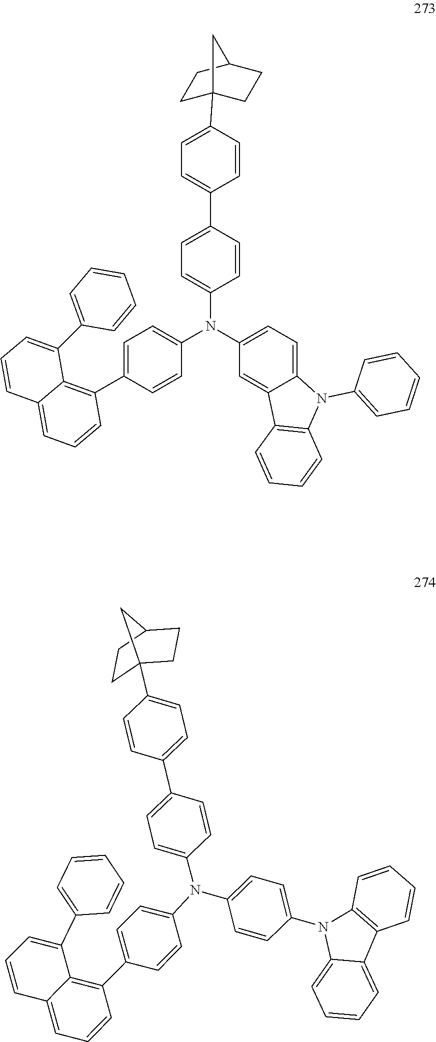 [SMILES: c1ccc(-c2cccc3cccc(-c4ccc(N(c5ccc(-c6ccc(C78CCC(CC7)C8)cc6)cc5)c5ccc6c(c5)c5ccccc5n6-c5ccccc5)cc4)c23)cc1 ; c1ccc(-c2cccc3cccc(-c4ccc(N(c5ccc(-c6ccc(C78CCC(CC7)C8)cc6)cc5)c5ccc(-n6c7ccccc7c7ccccc76)cc5)cc4)c23)cc1]

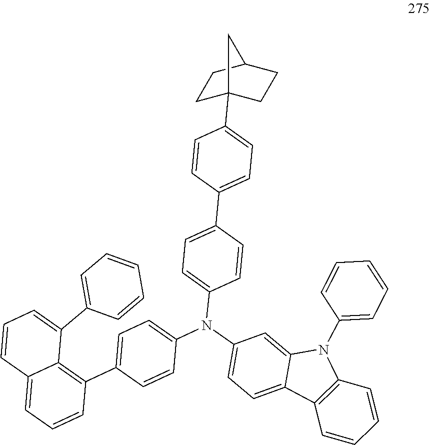 [SMILES: c1ccc(-c2cccc3cccc(-c4ccc(N(c5ccc(-c6ccc(C78CCC(CC7)C8)cc6)cc5)c5ccc6c7ccccc7n(-c7ccccc7)c6c5)cc4)c23)cc1]

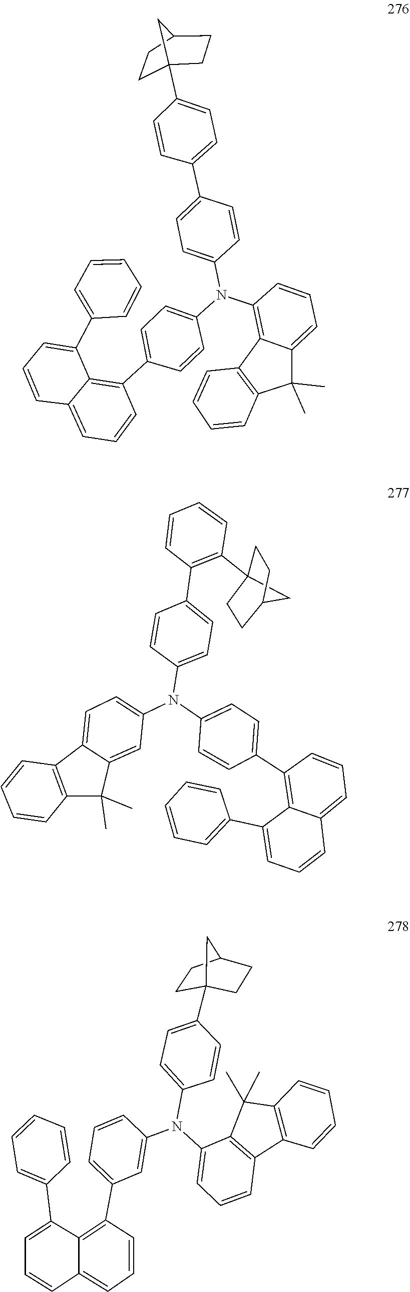 [SMILES: CC1(C)c2ccccc2-c2c(N(c3ccc(-c4ccc(C56CCC(CC5)C6)cc4)cc3)c3ccc(-c4cccc5cccc(-c6ccccc6)c45)cc3)cccc21 ; CC1(C)c2ccccc2-c2ccc(N(c3ccc(-c4ccccc4C45CCC(CC4)C5)cc3)c3ccc(-c4cccc5cccc(-c6ccccc6)c45)cc3)cc21 ; CC1(C)c2ccccc2-c2cccc(N(c3ccc(C45CCC(CC4)C5)cc3)c3cccc(-c4cccc5cccc(-c6ccccc6)c45)c3)c21]

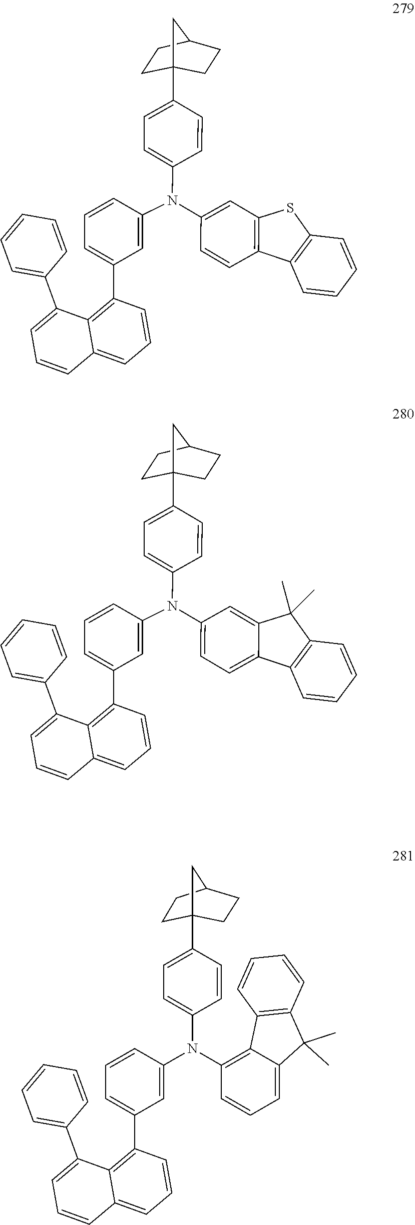 [SMILES: c1ccc(-c2cccc3cccc(-c4cccc(N(c5ccc(C67CCC(CC6)C7)cc5)c5ccc6c(c5)sc5ccccc56)c4)c23)cc1 ; CC1(C)c2ccccc2-c2ccc(N(c3ccc(C45CCC(CC4)C5)cc3)c3cccc(-c4cccc5cccc(-c6ccccc6)c45)c3)cc21 ; CC1(C)c2ccccc2-c2c(N(c3ccc(C45CCC(CC4)C5)cc3)c3cccc(-c4cccc5cccc(-c6ccccc6)c45)c3)cccc21]

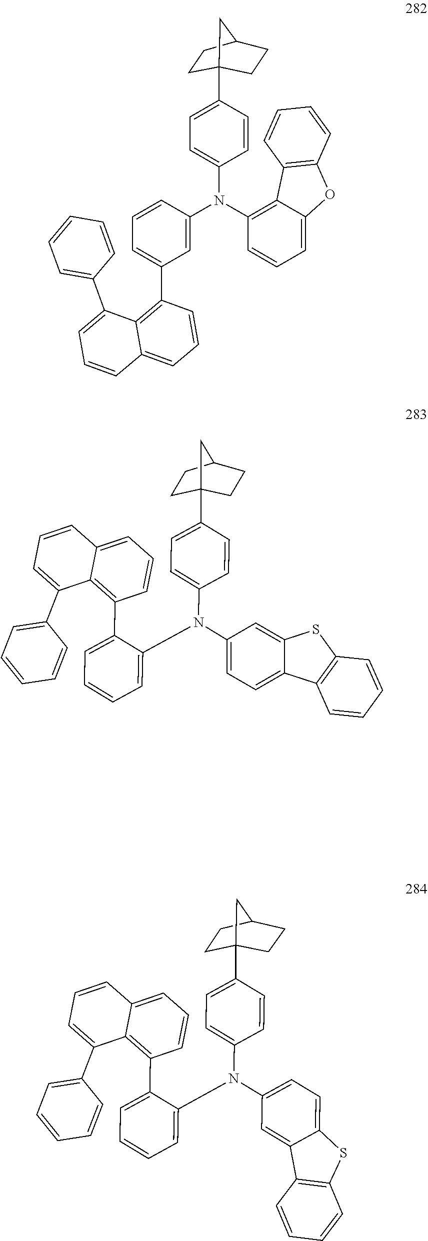 [SMILES: c1ccc(-c2cccc3cccc(-c4cccc(N(c5ccc(C67CCC(CC6)C7)cc5)c5cccc6oc7ccccc7c56)c4)c23)cc1 ; c1ccc(-c2cccc3cccc(-c4ccccc4N(c4ccc(C56CCC(CC5)C6)cc4)c4ccc5c(c4)sc4ccccc45)c23)cc1 ; c1ccc(-c2cccc3cccc(-c4ccccc4N(c4ccc(C56CCC(CC5)C6)cc4)c4ccc5sc6ccccc6c5c4)c23)cc1]

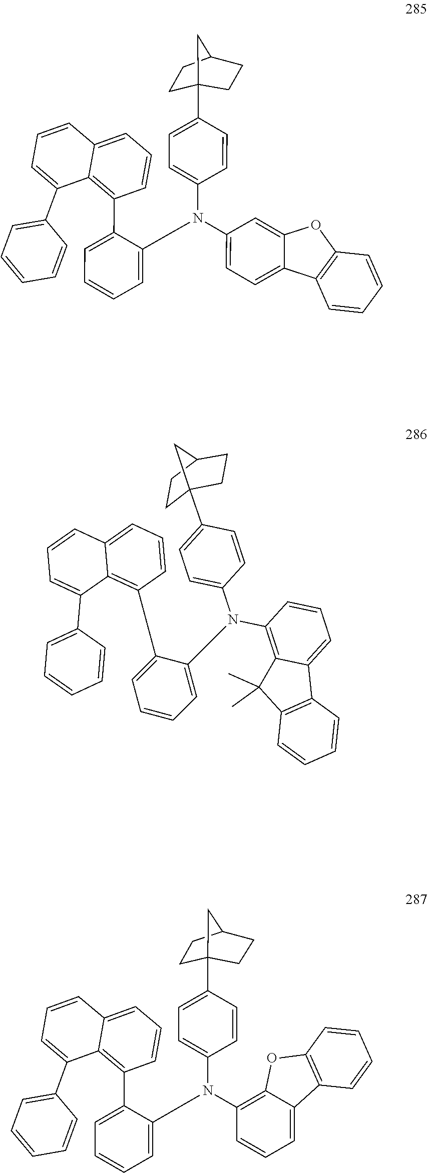 [SMILES: c1ccc(-c2cccc3cccc(-c4ccccc4N(c4ccc(C56CCC(CC5)C6)cc4)c4ccc5c(c4)oc4ccccc45)c23)cc1 ; CC1(C)c2ccccc2-c2cccc(N(c3ccc(C45CCC(CC4)C5)cc3)c3ccccc3-c3cccc4cccc(-c5ccccc5)c34)c21 ; c1ccc(-c2cccc3cccc(-c4ccccc4N(c4ccc(C56CCC(CC5)C6)cc4)c4cccc5c4oc4ccccc45)c23)cc1]

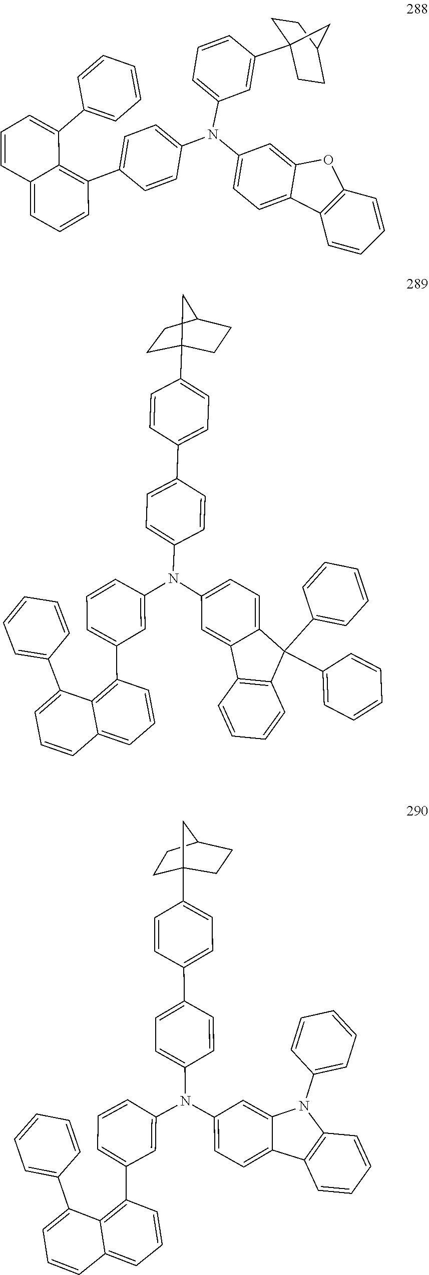 [SMILES: c1ccc(-c2cccc3cccc(-c4ccc(N(c5cccc(C67CCC(CC6)C7)c5)c5ccc6c(c5)oc5ccccc56)cc4)c23)cc1 ; c1ccc(-c2cccc3cccc(-c4cccc(N(c5ccc(-c6ccc(C78CCC(CC7)C8)cc6)cc5)c5ccc6c(c5)-c5ccccc5C6(c5ccccc5)c5ccccc5)c4)c23)cc1 ; c1ccc(-c2cccc3cccc(-c4cccc(N(c5ccc(-c6ccc(C78CCC(CC7)C8)cc6)cc5)c5ccc6c7ccccc7n(-c7ccccc7)c6c5)c4)c23)cc1]

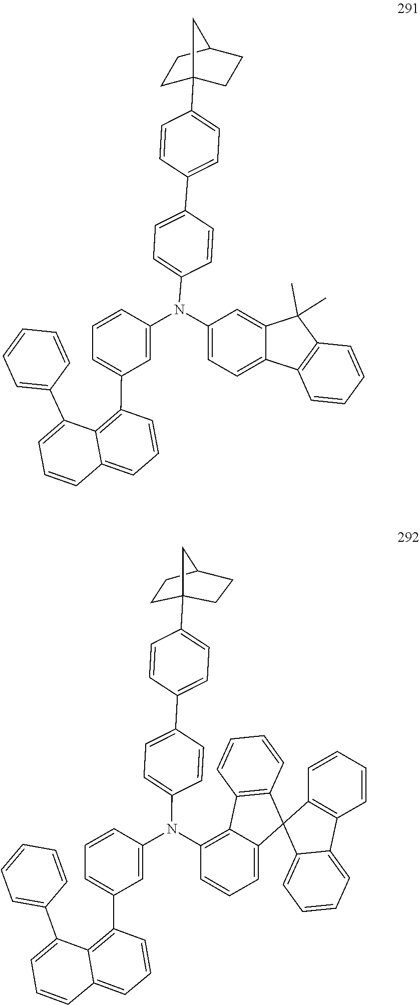 [SMILES: CC1(C)c2ccccc2-c2ccc(N(c3ccc(-c4ccc(C56CCC(CC5)C6)cc4)cc3)c3cccc(-c4cccc5cccc(-c6ccccc6)c45)c3)cc21 ; c1ccc(-c2cccc3cccc(-c4cccc(N(c5ccc(-c6ccc(C78CCC(CC7)C8)cc6)cc5)c5cccc6c5-c5ccccc5C65c6ccccc6-c6ccccc65)c4)c23)cc1]

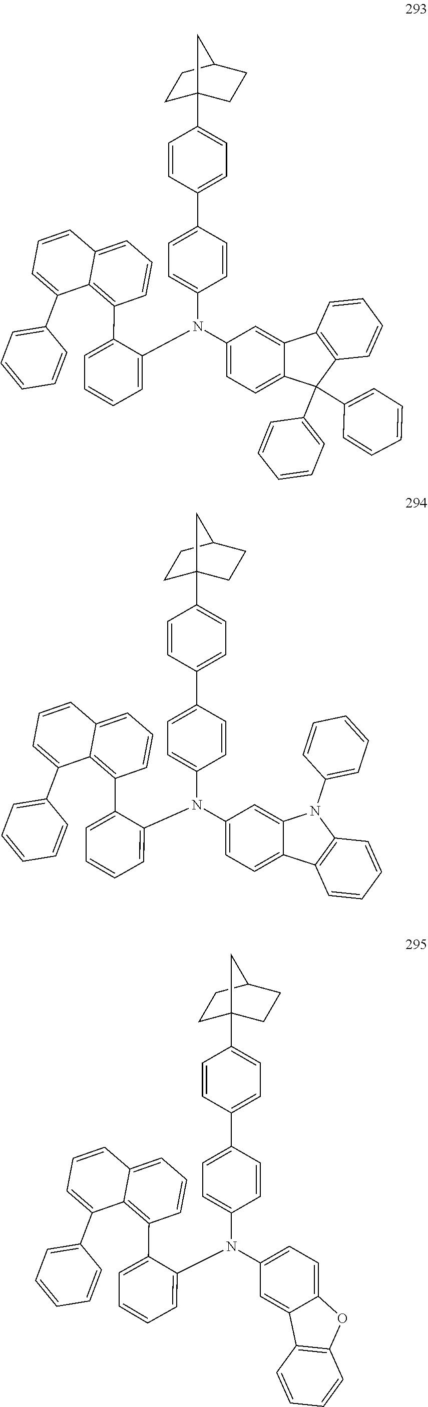 [SMILES: c1ccc(-c2cccc3cccc(-c4ccccc4N(c4ccc(-c5ccc(C67CCC(CC6)C7)cc5)cc4)c4ccc5c(c4)-c4ccccc4C5(c4ccccc4)c4ccccc4)c23)cc1 ; c1ccc(-c2cccc3cccc(-c4ccccc4N(c4ccc(-c5ccc(C67CCC(CC6)C7)cc5)cc4)c4ccc5c6ccccc6n(-c6ccccc6)c5c4)c23)cc1 ; c1ccc(-c2cccc3cccc(-c4ccccc4N(c4ccc(-c5ccc(C67CCC(CC6)C7)cc5)cc4)c4ccc5oc6ccccc6c5c4)c23)cc1]

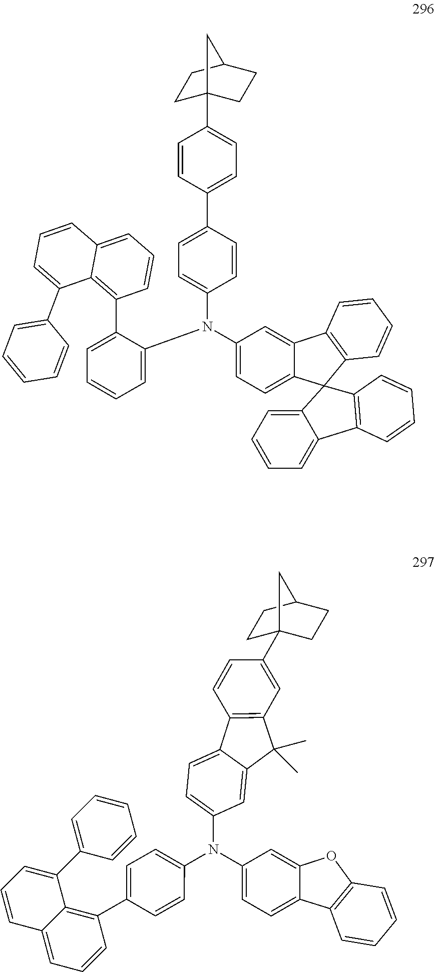 [SMILES: c1ccc(-c2cccc3cccc(-c4ccccc4N(c4ccc(-c5ccc(C67CCC(CC6)C7)cc5)cc4)c4ccc5c(c4)-c4ccccc4C54c5ccccc5-c5ccccc54)c23)cc1 ; CC1(C)c2cc(N(c3ccc(-c4cccc5cccc(-c6ccccc6)c45)cc3)c3ccc4c(c3)oc3ccccc34)ccc2-c2ccc(C34CCC(CC3)C4)cc21]

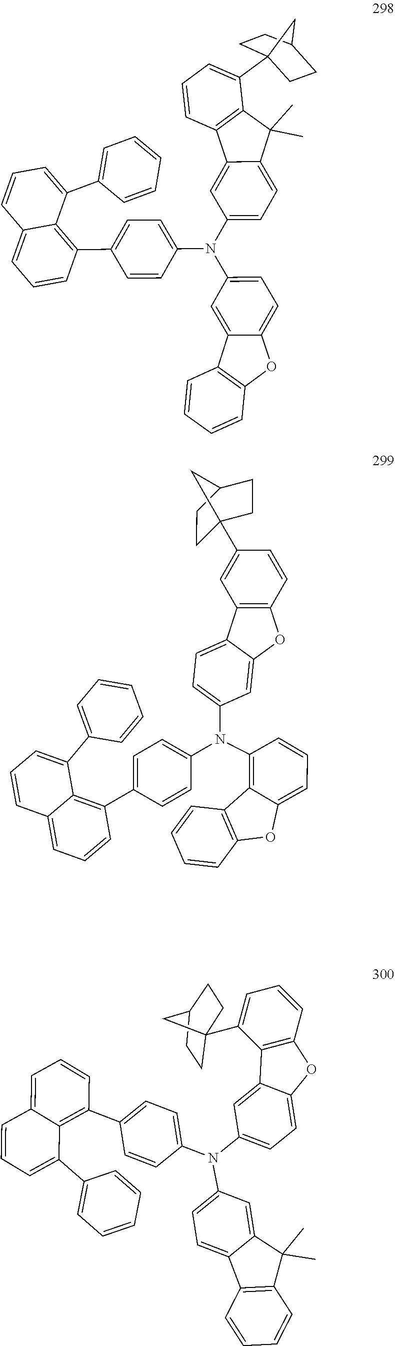

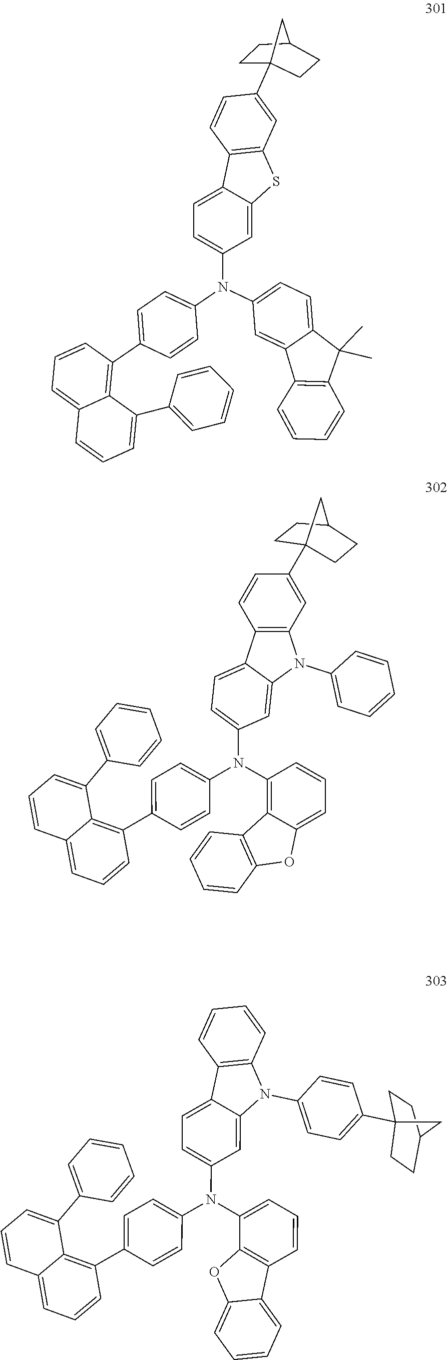 [SMILES: CC1(C)c2ccccc2-c2cc(N(c3ccc(-c4cccc5cccc(-c6ccccc6)c45)cc3)c3ccc4c(c3)sc3cc(C56CCC(CC5)C6)ccc34)ccc21 ; c1ccc(-c2cccc3cccc(-c4ccc(N(c5ccc6c7ccc(C89CCC(CC8)C9)cc7n(-c7ccccc7)c6c5)c5cccc6oc7ccccc7c56)cc4)c23)cc1 ; c1ccc(-c2cccc3cccc(-c4ccc(N(c5ccc6c7ccccc7n(-c7ccc(C89CCC(CC8)C9)cc7)c6c5)c5cccc6c5oc5ccccc56)cc4)c23)cc1]

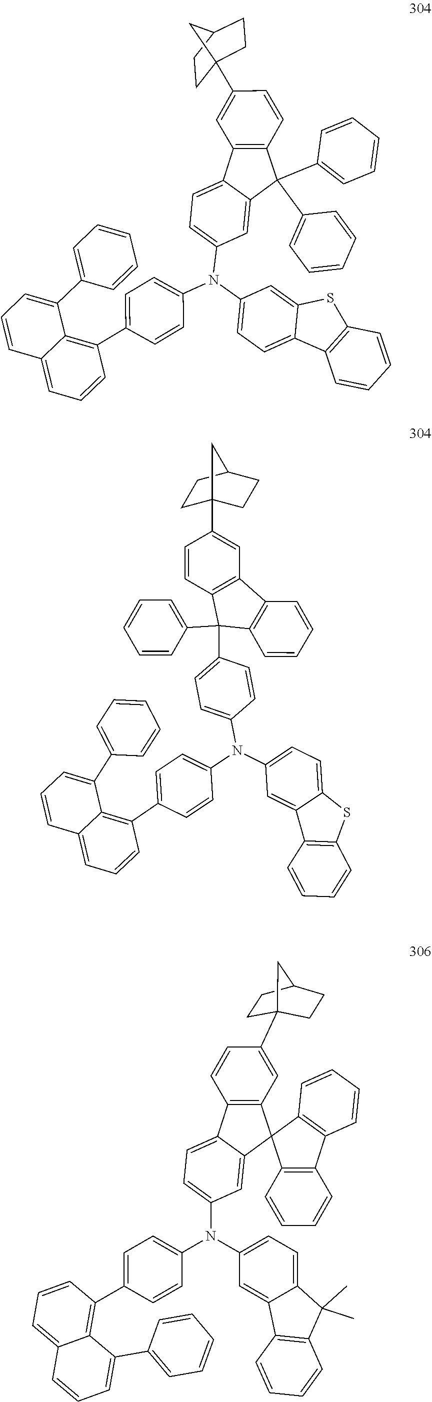 [SMILES: c1ccc(-c2cccc3cccc(-c4ccc(N(c5ccc6c(c5)C(c5ccccc5)(c5ccccc5)c5ccc(C78CCC(CC7)C8)cc5-6)c5ccc6c(c5)sc5ccccc56)cc4)c23)cc1 ; c1ccc(-c2cccc3cccc(-c4ccc(N(c5ccc(C6(c7ccccc7)c7ccccc7-c7cc(C89CCC(CC8)C9)ccc76)cc5)c5ccc6sc7ccccc7c6c5)cc4)c23)cc1 ; CC1(C)c2ccccc2-c2cc(N(c3ccc(-c4cccc5cccc(-c6ccccc6)c45)cc3)c3ccc4c(c3)C3(c5ccccc5-c5ccccc53)c3cc(C56CCC(CC5)C6)ccc3-4)ccc21]

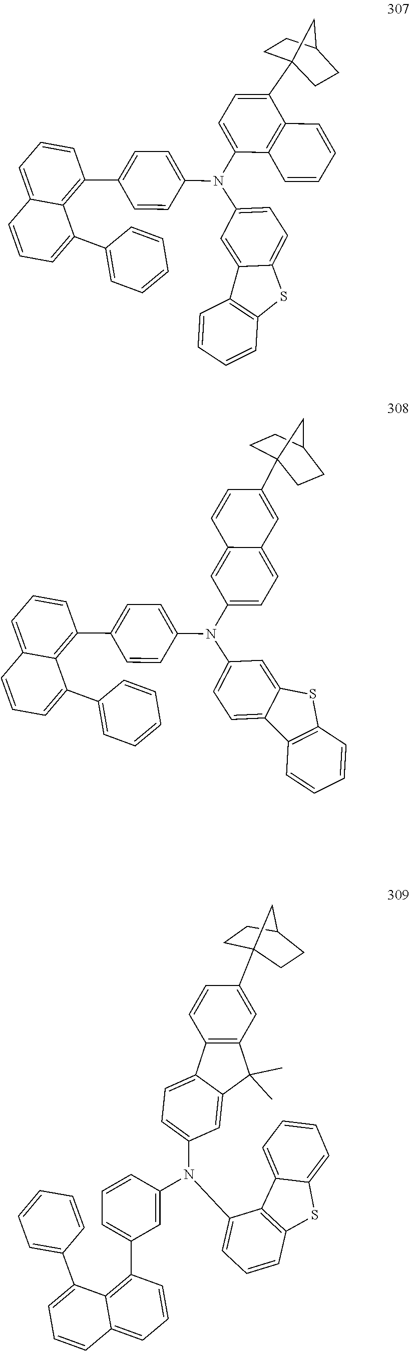 [SMILES: c1ccc(-c2cccc3cccc(-c4ccc(N(c5ccc6sc7ccccc7c6c5)c5ccc(C67CCC(CC6)C7)c6ccccc56)cc4)c23)cc1 ; c1ccc(-c2cccc3cccc(-c4ccc(N(c5ccc6cc(C78CCC(CC7)C8)ccc6c5)c5ccc6c(c5)sc5ccccc56)cc4)c23)cc1 ; CC1(C)c2cc(N(c3cccc(-c4cccc5cccc(-c6ccccc6)c45)c3)c3cccc4sc5ccccc5c34)ccc2-c2ccc(C34CCC(CC3)C4)cc21]

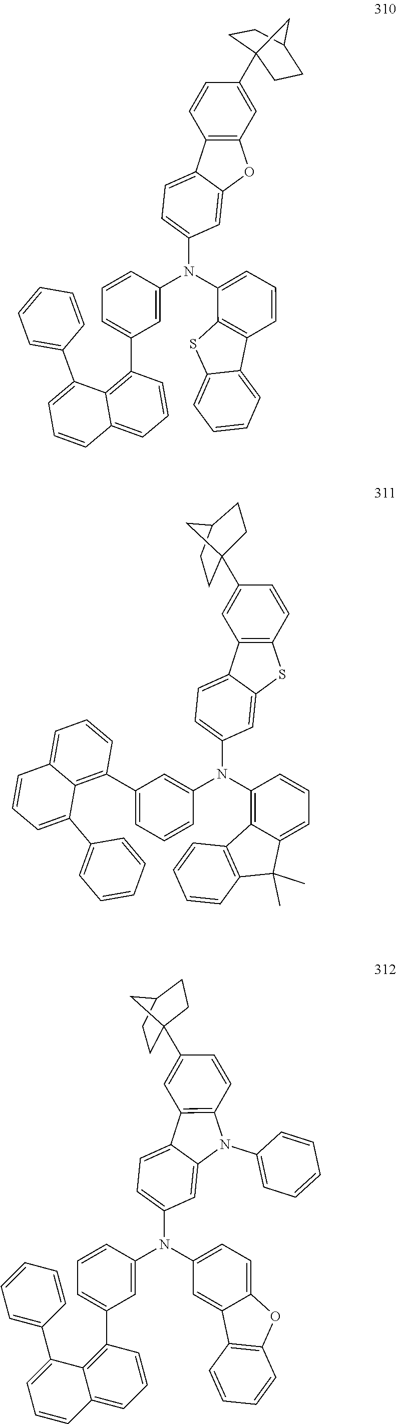 [SMILES: c1ccc(-c2cccc3cccc(-c4cccc(N(c5ccc6c(c5)oc5cc(C78CCC(CC7)C8)ccc56)c5cccc6c5sc5ccccc56)c4)c23)cc1 ; CC1(C)c2ccccc2-c2c(N(c3cccc(-c4cccc5cccc(-c6ccccc6)c45)c3)c3ccc4c(c3)sc3ccc(C56CCC(CC5)C6)cc34)cccc21 ; c1ccc(-c2cccc3cccc(-c4cccc(N(c5ccc6oc7ccccc7c6c5)c5ccc6c7cc(C89CCC(CC8)C9)ccc7n(-c7ccccc7)c6c5)c4)c23)cc1]

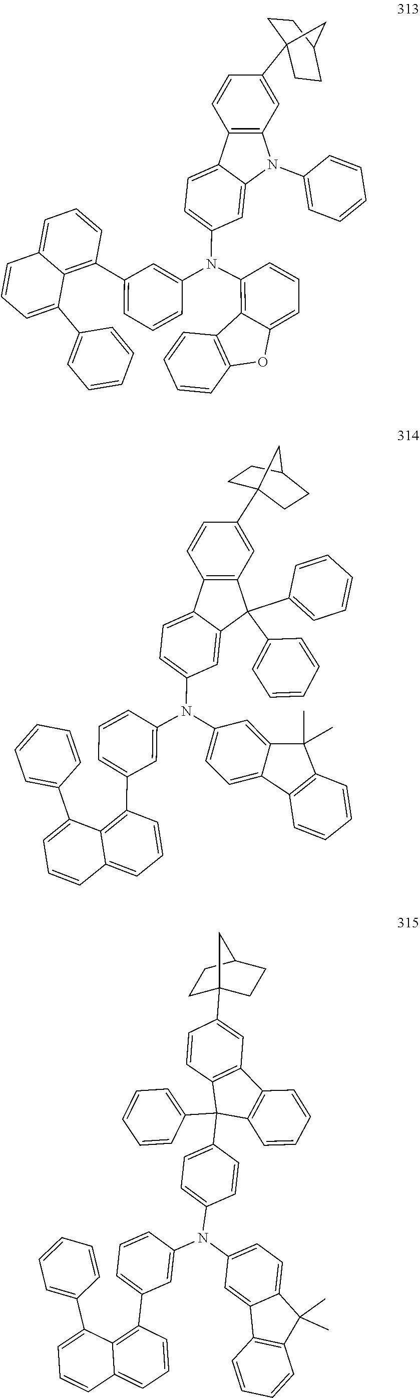 [SMILES: c1ccc(-c2cccc3cccc(-c4cccc(N(c5ccc6c7ccc(C89CCC(CC8)C9)cc7n(-c7ccccc7)c6c5)c5cccc6oc7ccccc7c56)c4)c23)cc1 ; CC1(C)c2ccccc2-c2ccc(N(c3cccc(-c4cccc5cccc(-c6ccccc6)c45)c3)c3ccc4c(c3)C(c3ccccc3)(c3ccccc3)c3cc(C56CCC(CC5)C6)ccc3-4)cc21 ; CC1(C)c2ccccc2-c2cc(N(c3ccc(C4(c5ccccc5)c5ccccc5-c5cc(C67CCC(CC6)C7)ccc54)cc3)c3cccc(-c4cccc5cccc(-c6ccccc6)c45)c3)ccc21]

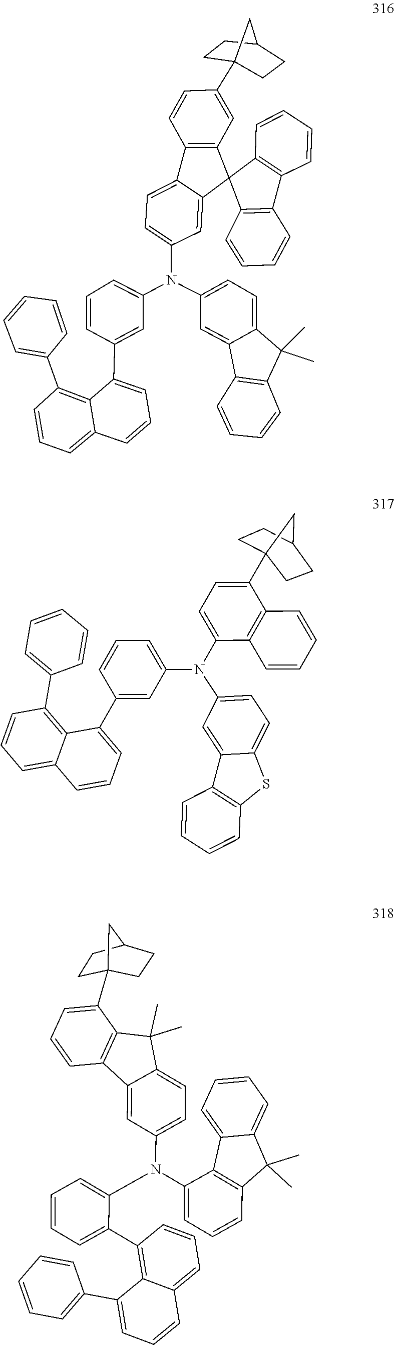 [SMILES: CC1(C)c2ccccc2-c2cc(N(c3cccc(-c4cccc5cccc(-c6ccccc6)c45)c3)c3ccc4c(c3)C3(c5ccccc5-c5ccccc53)c3cc(C56CCC(CC5)C6)ccc3-4)ccc21 ; c1ccc(-c2cccc3cccc(-c4cccc(N(c5ccc6sc7ccccc7c6c5)c5ccc(C67CCC(CC6)C7)c6ccccc56)c4)c23)cc1 ; CC1(C)c2ccccc2-c2c(N(c3ccc4c(c3)-c3cccc(C56CCC(CC5)C6)c3C4(C)C)c3ccccc3-c3cccc4cccc(-c5ccccc5)c34)cccc21]

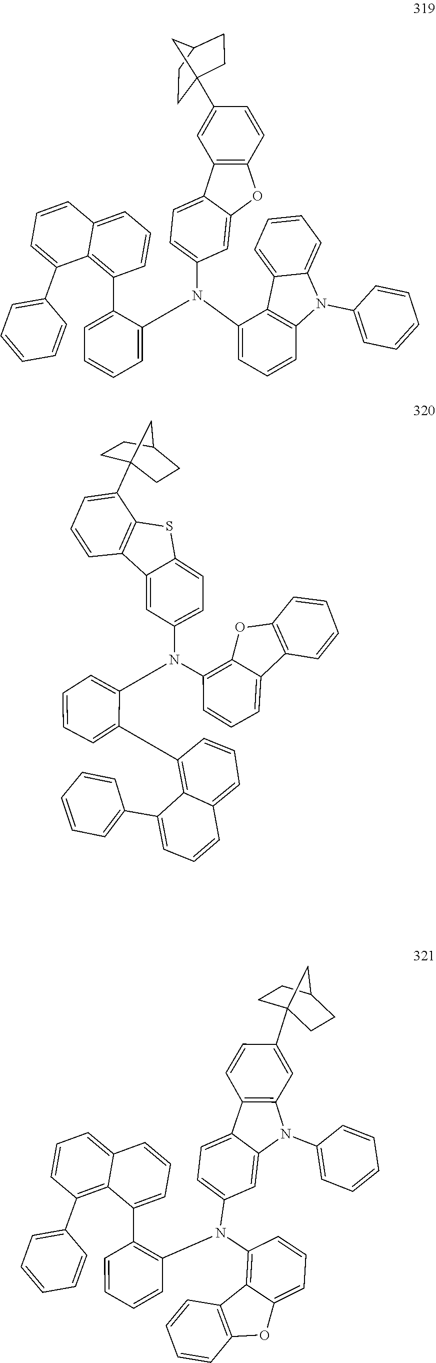 [SMILES: c1ccc(-c2cccc3cccc(-c4ccccc4N(c4ccc5c(c4)oc4ccc(C67CCC(CC6)C7)cc45)c4cccc5c4c4ccccc4n5-c4ccccc4)c23)cc1 ; c1ccc(-c2cccc3cccc(-c4ccccc4N(c4ccc5sc6c(C78CCC(CC7)C8)cccc6c5c4)c4cccc5c4oc4ccccc45)c23)cc1 ; c1ccc(-c2cccc3cccc(-c4ccccc4N(c4ccc5c6ccc(C78CCC(CC7)C8)cc6n(-c6ccccc6)c5c4)c4cccc5oc6ccccc6c45)c23)cc1]

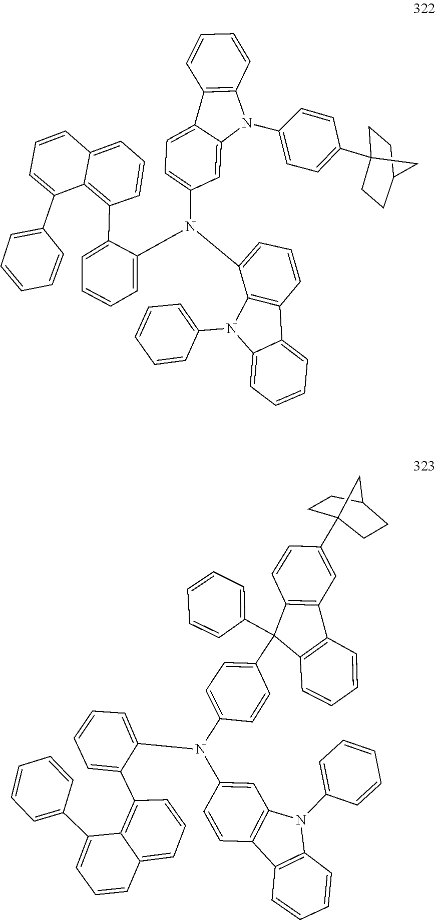 [SMILES: c1ccc(-c2cccc3cccc(-c4ccccc4N(c4ccc5c6ccccc6n(-c6ccc(C78CCC(CC7)C8)cc6)c5c4)c4cccc5c6ccccc6n(-c6ccccc6)c45)c23)cc1 ; c1ccc(-c2cccc3cccc(-c4ccccc4N(c4ccc(C5(c6ccccc6)c6ccccc6-c6cc(C78CCC(CC7)C8)ccc65)cc4)c4ccc5c6ccccc6n(-c6ccccc6)c5c4)c23)cc1]

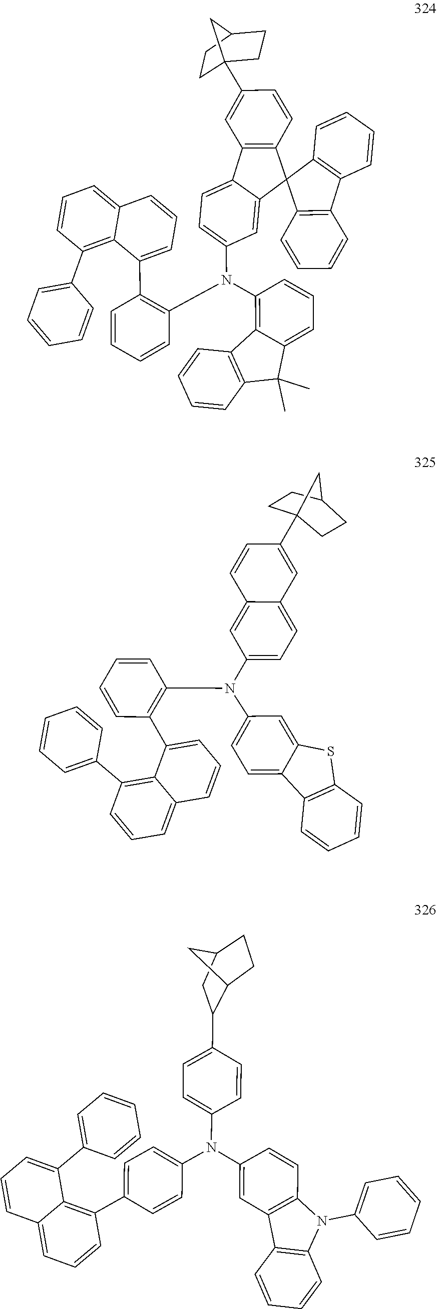 [SMILES: CC1(C)c2ccccc2-c2c(N(c3ccc4c(c3)C3(c5ccccc5-c5ccccc53)c3ccc(C56CCC(CC5)C6)cc3-4)c3ccccc3-c3cccc4cccc(-c5ccccc5)c34)cccc21 ; c1ccc(-c2cccc3cccc(-c4ccccc4N(c4ccc5cc(C67CCC(CC6)C7)ccc5c4)c4ccc5c(c4)sc4ccccc45)c23)cc1 ; c1ccc(-c2cccc3cccc(-c4ccc(N(c5ccc(C6CC7CCC6C7)cc5)c5ccc6c(c5)c5ccccc5n6-c5ccccc5)cc4)c23)cc1]

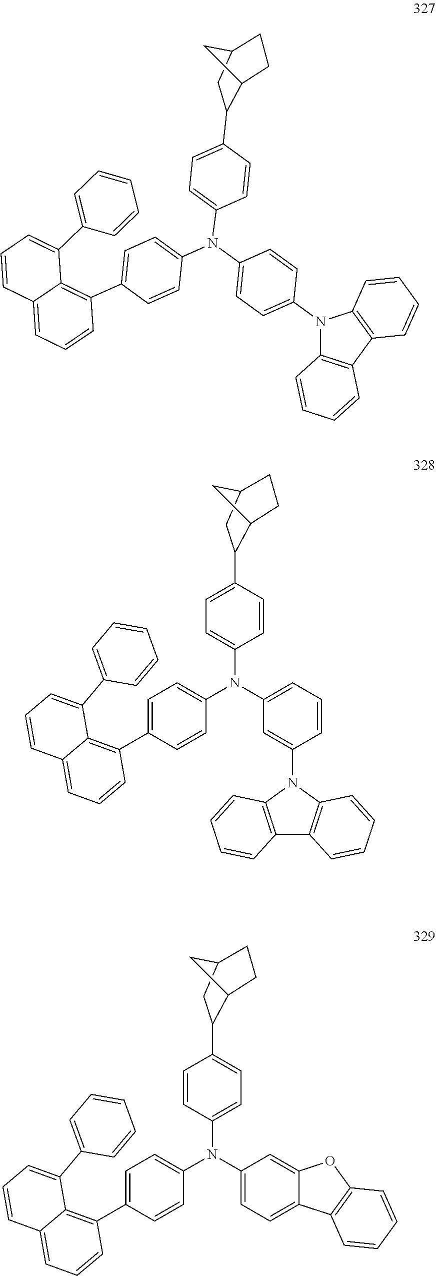 [SMILES: c1ccc(-c2cccc3cccc(-c4ccc(N(c5ccc(C6CC7CCC6C7)cc5)c5ccc(-n6c7ccccc7c7ccccc76)cc5)cc4)c23)cc1 ; c1ccc(-c2cccc3cccc(-c4ccc(N(c5ccc(C6CC7CCC6C7)cc5)c5cccc(-n6c7ccccc7c7ccccc76)c5)cc4)c23)cc1 ; c1ccc(-c2cccc3cccc(-c4ccc(N(c5ccc(C6CC7CCC6C7)cc5)c5ccc6c(c5)oc5ccccc56)cc4)c23)cc1]

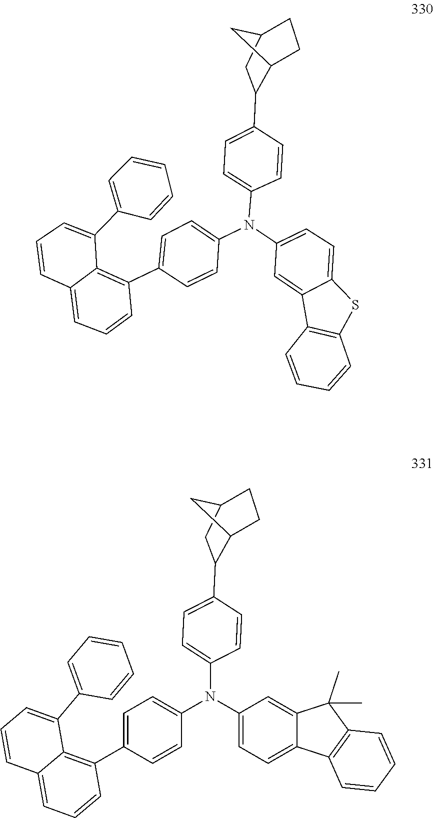 [SMILES: c1ccc(-c2cccc3cccc(-c4ccc(N(c5ccc(C6CC7CCC6C7)cc5)c5ccc6sc7ccccc7c6c5)cc4)c23)cc1 ; CC1(C)c2ccccc2-c2ccc(N(c3ccc(-c4cccc5cccc(-c6ccccc6)c45)cc3)c3ccc(C4CC5CCC4C5)cc3)cc21]

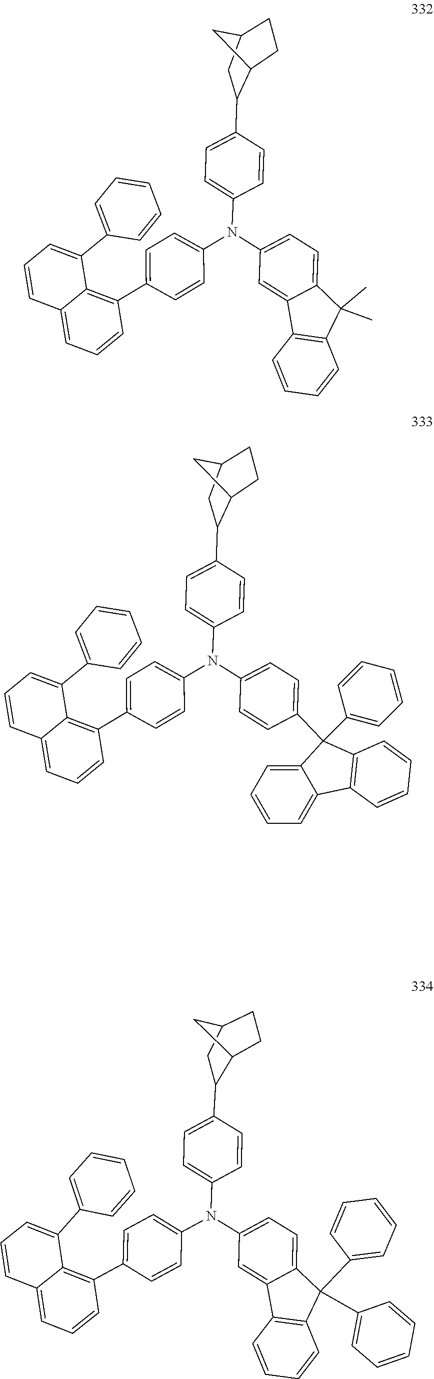 [SMILES: CC1(C)c2ccccc2-c2cc(N(c3ccc(-c4cccc5cccc(-c6ccccc6)c45)cc3)c3ccc(C4CC5CCC4C5)cc3)ccc21 ; c1ccc(-c2cccc3cccc(-c4ccc(N(c5ccc(C6CC7CCC6C7)cc5)c5ccc(C6(c7ccccc7)c7ccccc7-c7ccccc76)cc5)cc4)c23)cc1 ; c1ccc(-c2cccc3cccc(-c4ccc(N(c5ccc(C6CC7CCC6C7)cc5)c5ccc6c(c5)-c5ccccc5C6(c5ccccc5)c5ccccc5)cc4)c23)cc1]

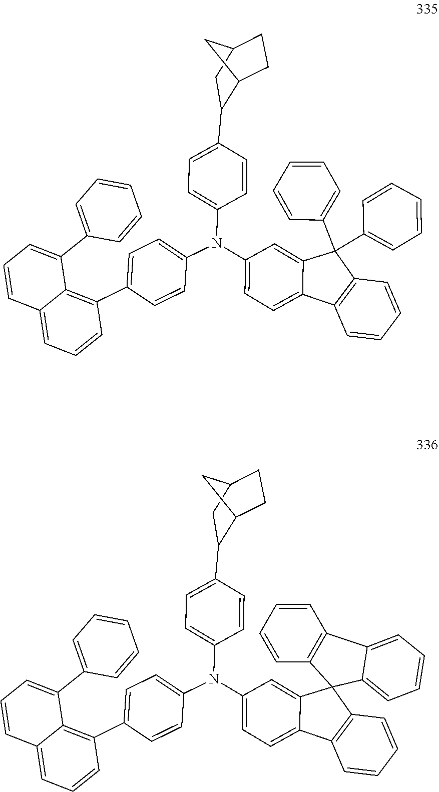 [SMILES: c1ccc(-c2cccc3cccc(-c4ccc(N(c5ccc(C6CC7CCC6C7)cc5)c5ccc6c(c5)C(c5ccccc5)(c5ccccc5)c5ccccc5-6)cc4)c23)cc1 ; c1ccc(-c2cccc3cccc(-c4ccc(N(c5ccc(C6CC7CCC6C7)cc5)c5ccc6c(c5)C5(c7ccccc7-c7ccccc75)c5ccccc5-6)cc4)c23)cc1]

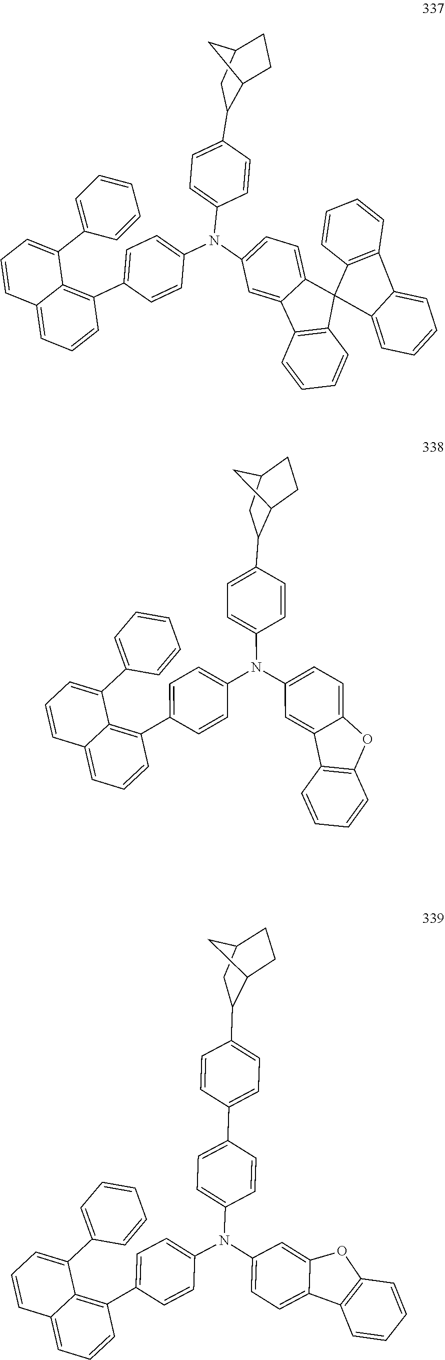 [SMILES: c1ccc(-c2cccc3cccc(-c4ccc(N(c5ccc(C6CC7CCC6C7)cc5)c5ccc6c(c5)-c5ccccc5C65c6ccccc6-c6ccccc65)cc4)c23)cc1 ; c1ccc(-c2cccc3cccc(-c4ccc(N(c5ccc(C6CC7CCC6C7)cc5)c5ccc6oc7ccccc7c6c5)cc4)c23)cc1 ; c1ccc(-c2cccc3cccc(-c4ccc(N(c5ccc(-c6ccc(C7CC8CCC7C8)cc6)cc5)c5ccc6c(c5)oc5ccccc56)cc4)c23)cc1]

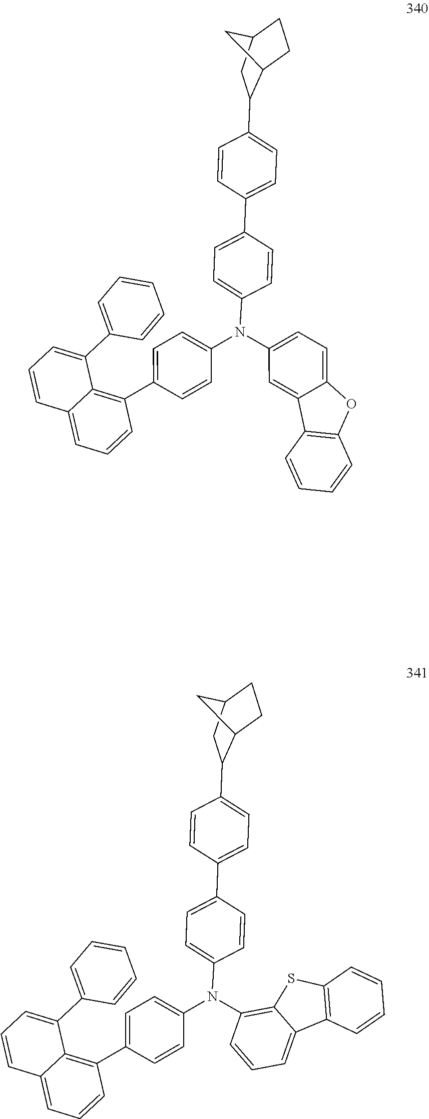 [SMILES: c1ccc(-c2cccc3cccc(-c4ccc(N(c5ccc(-c6ccc(C7CC8CCC7C8)cc6)cc5)c5ccc6oc7ccccc7c6c5)cc4)c23)cc1 ; c1ccc(-c2cccc3cccc(-c4ccc(N(c5ccc(-c6ccc(C7CC8CCC7C8)cc6)cc5)c5cccc6c5sc5ccccc56)cc4)c23)cc1]

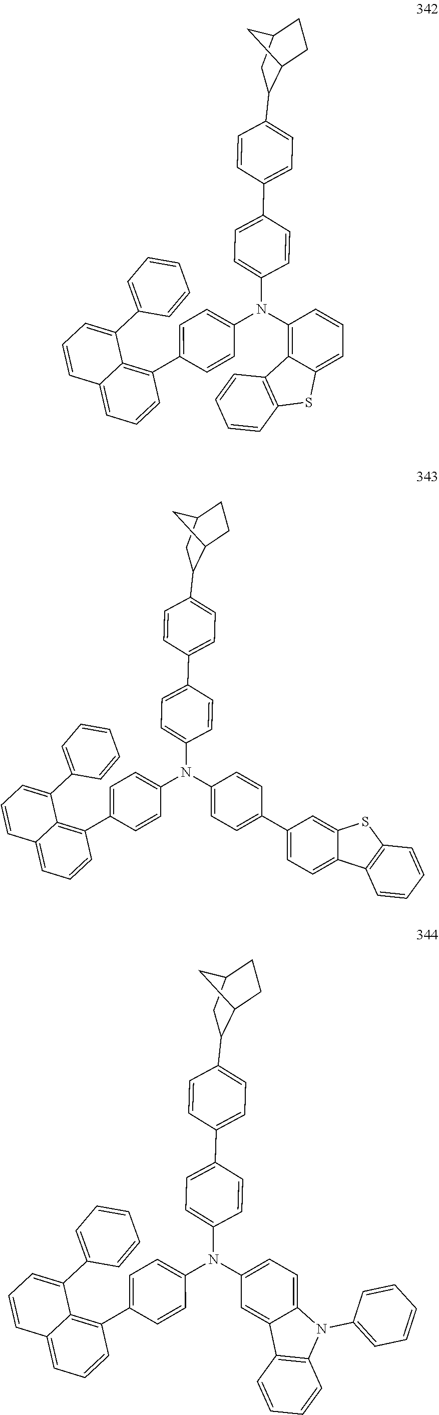 [SMILES: c1ccc(-c2cccc3cccc(-c4ccc(N(c5ccc(-c6ccc(C7CC8CCC7C8)cc6)cc5)c5cccc6sc7ccccc7c56)cc4)c23)cc1 ; c1ccc(-c2cccc3cccc(-c4ccc(N(c5ccc(-c6ccc(C7CC8CCC7C8)cc6)cc5)c5ccc(-c6ccc7c(c6)sc6ccccc67)cc5)cc4)c23)cc1 ; c1ccc(-c2cccc3cccc(-c4ccc(N(c5ccc(-c6ccc(C7CC8CCC7C8)cc6)cc5)c5ccc6c(c5)c5ccccc5n6-c5ccccc5)cc4)c23)cc1]

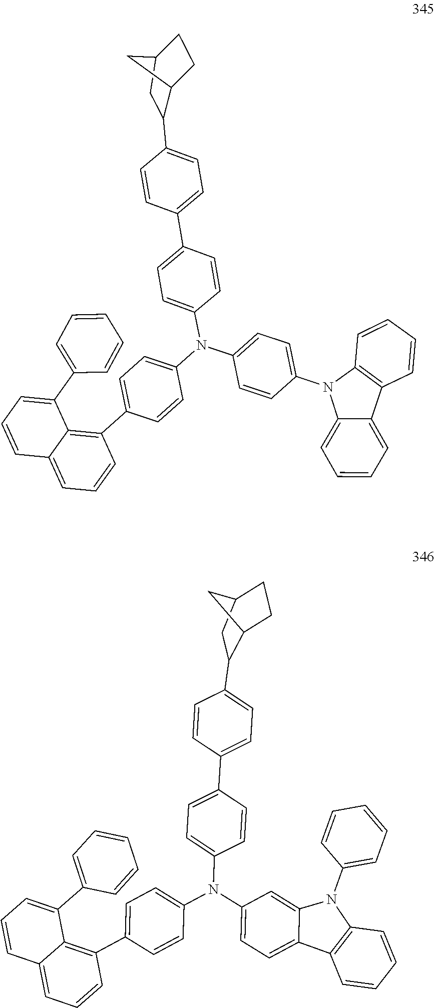 [SMILES: c1ccc(-c2cccc3cccc(-c4ccc(N(c5ccc(-c6ccc(C7CC8CCC7C8)cc6)cc5)c5ccc(-n6c7ccccc7c7ccccc76)cc5)cc4)c23)cc1 ; c1ccc(-c2cccc3cccc(-c4ccc(N(c5ccc(-c6ccc(C7CC8CCC7C8)cc6)cc5)c5ccc6c7ccccc7n(-c7ccccc7)c6c5)cc4)c23)cc1]

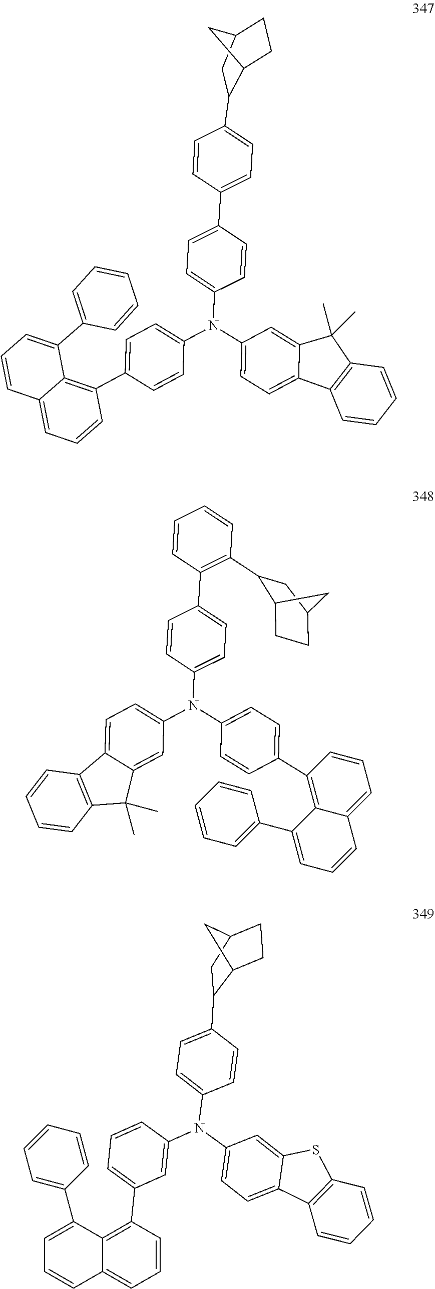 [SMILES: CC1(C)c2ccccc2-c2ccc(N(c3ccc(-c4ccc(C5CC6CCC5C6)cc4)cc3)c3ccc(-c4cccc5cccc(-c6ccccc6)c45)cc3)cc21 ; CC1(C)c2ccccc2-c2ccc(N(c3ccc(-c4ccccc4C4CC5CCC4C5)cc3)c3ccc(-c4cccc5cccc(-c6ccccc6)c45)cc3)cc21 ; c1ccc(-c2cccc3cccc(-c4cccc(N(c5ccc(C6CC7CCC6C7)cc5)c5ccc6c(c5)sc5ccccc56)c4)c23)cc1]

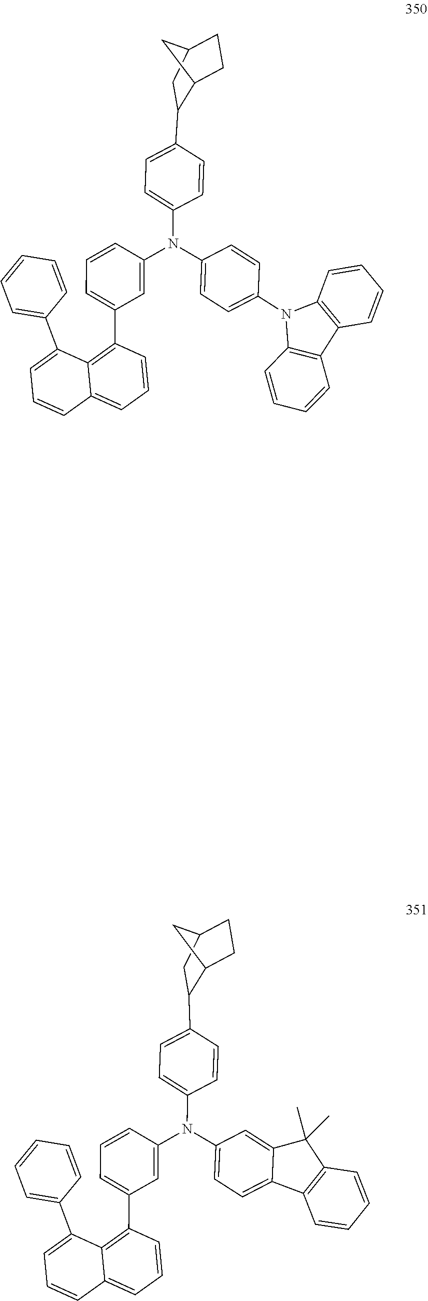 [SMILES: c1ccc(-c2cccc3cccc(-c4cccc(N(c5ccc(C6CC7CCC6C7)cc5)c5ccc(-n6c7ccccc7c7ccccc76)cc5)c4)c23)cc1 ; CC1(C)c2ccccc2-c2ccc(N(c3ccc(C4CC5CCC4C5)cc3)c3cccc(-c4cccc5cccc(-c6ccccc6)c45)c3)cc21]

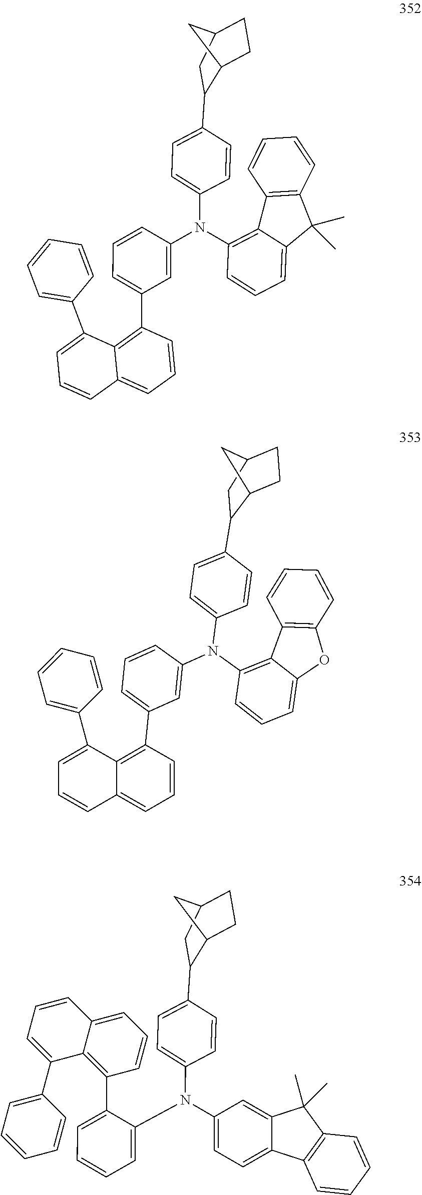 [SMILES: CC1(C)c2ccccc2-c2c(N(c3ccc(C4CC5CCC4C5)cc3)c3cccc(-c4cccc5cccc(-c6ccccc6)c45)c3)cccc21 ; c1ccc(-c2cccc3cccc(-c4cccc(N(c5ccc(C6CC7CCC6C7)cc5)c5cccc6oc7ccccc7c56)c4)c23)cc1 ; CC1(C)c2ccccc2-c2ccc(N(c3ccc(C4CC5CCC4C5)cc3)c3ccccc3-c3cccc4cccc(-c5ccccc5)c34)cc21]

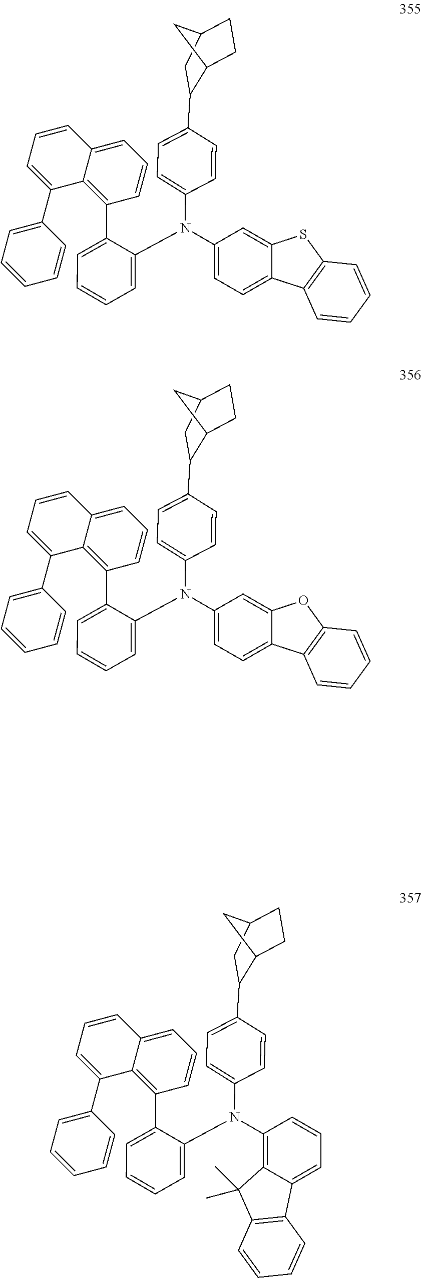 [SMILES: c1ccc(-c2cccc3cccc(-c4ccccc4N(c4ccc(C5CC6CCC5C6)cc4)c4ccc5c(c4)sc4ccccc45)c23)cc1 ; c1ccc(-c2cccc3cccc(-c4ccccc4N(c4ccc(C5CC6CCC5C6)cc4)c4ccc5c(c4)oc4ccccc45)c23)cc1 ; CC1(C)c2ccccc2-c2cccc(N(c3ccc(C4CC5CCC4C5)cc3)c3ccccc3-c3cccc4cccc(-c5ccccc5)c34)c21]

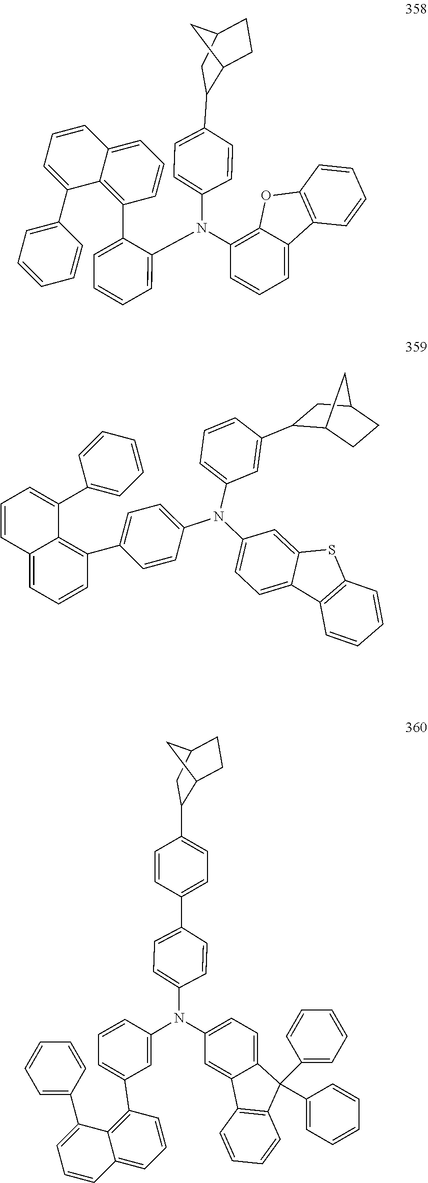 [SMILES: c1ccc(-c2cccc3cccc(-c4ccccc4N(c4ccc(C5CC6CCC5C6)cc4)c4cccc5c4oc4ccccc45)c23)cc1 ; c1ccc(-c2cccc3cccc(-c4ccc(N(c5cccc(C6CC7CCC6C7)c5)c5ccc6c(c5)sc5ccccc56)cc4)c23)cc1 ; c1ccc(-c2cccc3cccc(-c4cccc(N(c5ccc(-c6ccc(C7CC8CCC7C8)cc6)cc5)c5ccc6c(c5)-c5ccccc5C6(c5ccccc5)c5ccccc5)c4)c23)cc1]

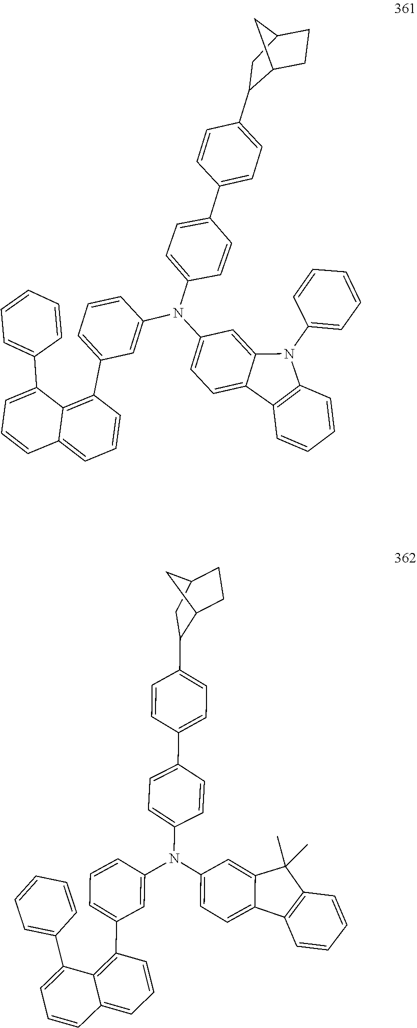 [SMILES: c1ccc(-c2cccc3cccc(-c4cccc(N(c5ccc(-c6ccc(C7CC8CCC7C8)cc6)cc5)c5ccc6c7ccccc7n(-c7ccccc7)c6c5)c4)c23)cc1 ; CC1(C)c2ccccc2-c2ccc(N(c3ccc(-c4ccc(C5CC6CCC5C6)cc4)cc3)c3cccc(-c4cccc5cccc(-c6ccccc6)c45)c3)cc21]

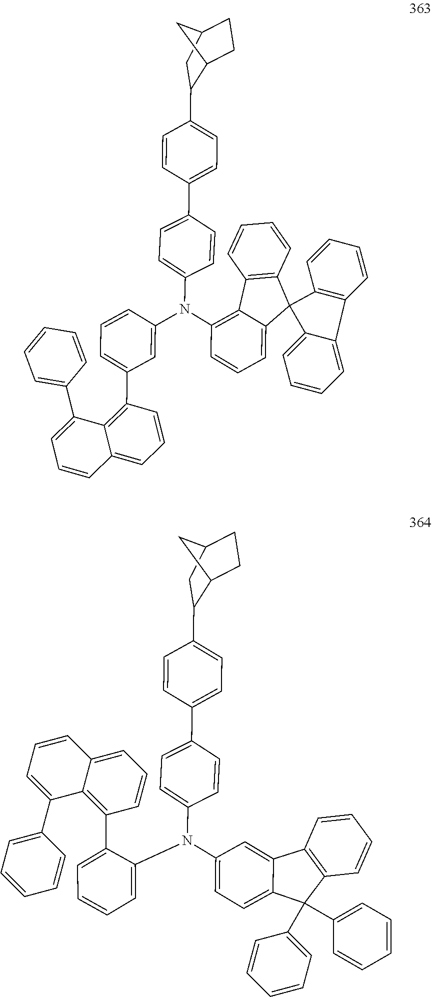 [SMILES: c1ccc(-c2cccc3cccc(-c4cccc(N(c5ccc(-c6ccc(C7CC8CCC7C8)cc6)cc5)c5cccc6c5-c5ccccc5C65c6ccccc6-c6ccccc65)c4)c23)cc1 ; c1ccc(-c2cccc3cccc(-c4ccccc4N(c4ccc(-c5ccc(C6CC7CCC6C7)cc5)cc4)c4ccc5c(c4)-c4ccccc4C5(c4ccccc4)c4ccccc4)c23)cc1]

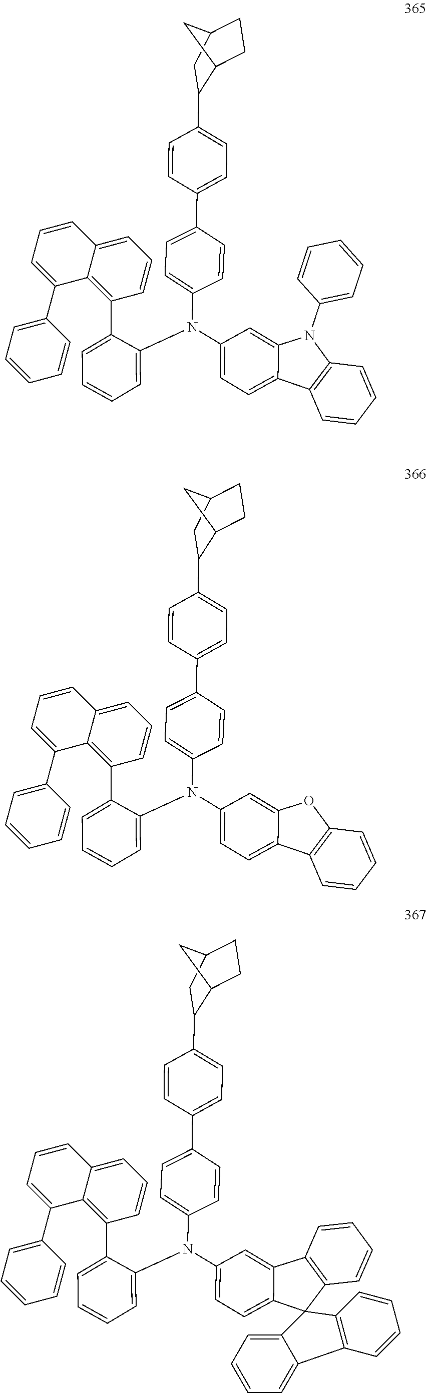 [SMILES: c1ccc(-c2cccc3cccc(-c4ccccc4N(c4ccc(-c5ccc(C6CC7CCC6C7)cc5)cc4)c4ccc5c6ccccc6n(-c6ccccc6)c5c4)c23)cc1 ; c1ccc(-c2cccc3cccc(-c4ccccc4N(c4ccc(-c5ccc(C6CC7CCC6C7)cc5)cc4)c4ccc5c(c4)oc4ccccc45)c23)cc1 ; c1ccc(-c2cccc3cccc(-c4ccccc4N(c4ccc(-c5ccc(C6CC7CCC6C7)cc5)cc4)c4ccc5c(c4)-c4ccccc4C54c5ccccc5-c5ccccc54)c23)cc1]

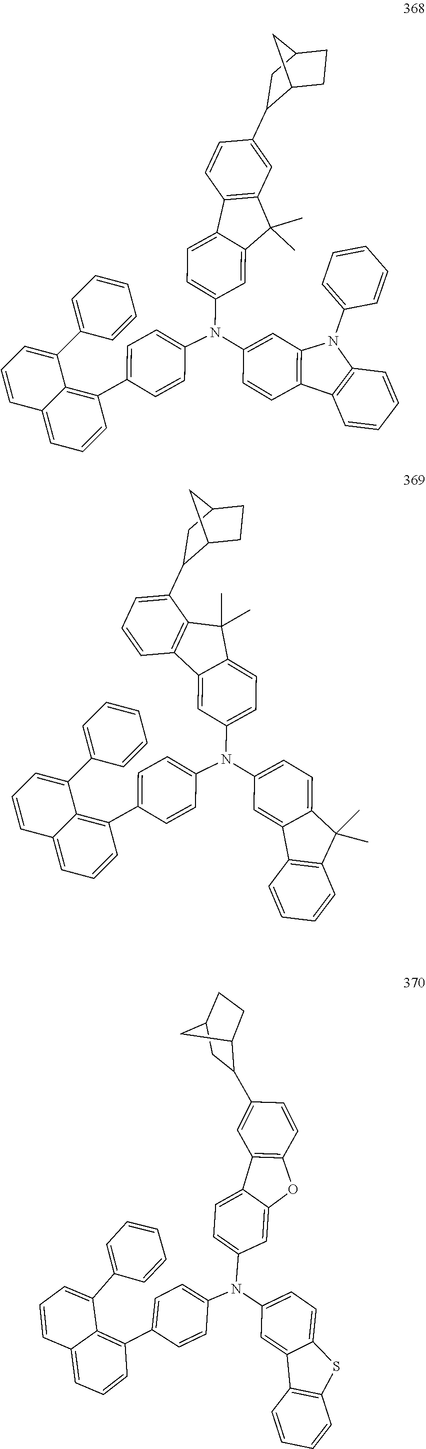 [SMILES: CC1(C)c2cc(C3CC4CCC3C4)ccc2-c2ccc(N(c3ccc(-c4cccc5cccc(-c6ccccc6)c45)cc3)c3ccc4c5ccccc5n(-c5ccccc5)c4c3)cc21 ; CC1(C)c2ccccc2-c2cc(N(c3ccc(-c4cccc5cccc(-c6ccccc6)c45)cc3)c3ccc4c(c3)-c3cccc(C5CC6CCC5C6)c3C4(C)C)ccc21 ; c1ccc(-c2cccc3cccc(-c4ccc(N(c5ccc6c(c5)oc5ccc(C7CC8CCC7C8)cc56)c5ccc6sc7ccccc7c6c5)cc4)c23)cc1]

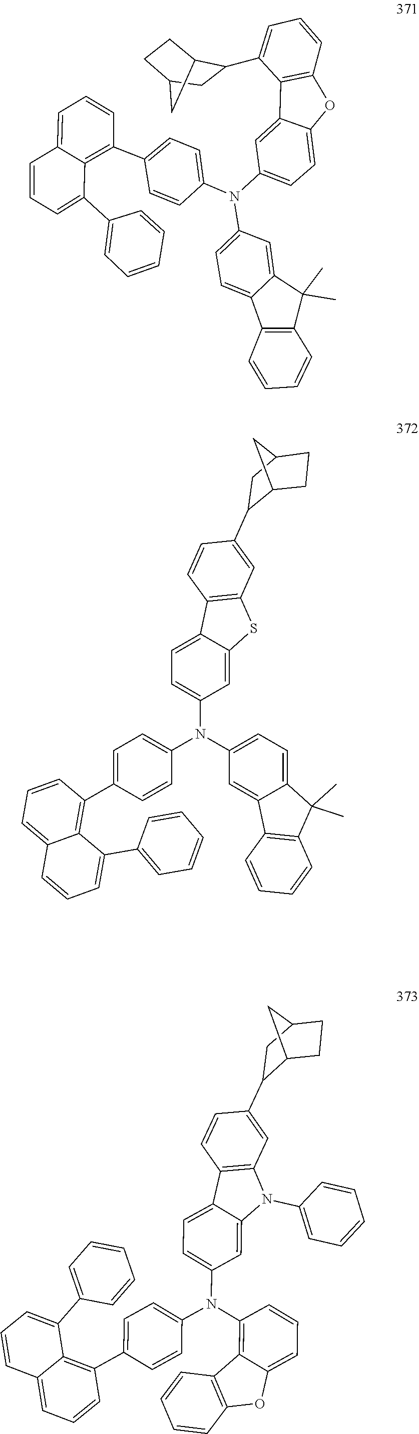 [SMILES: CC1(C)c2ccccc2-c2ccc(N(c3ccc(-c4cccc5cccc(-c6ccccc6)c45)cc3)c3ccc4oc5cccc(C6CC7CCC6C7)c5c4c3)cc21 ; CC1(C)c2ccccc2-c2cc(N(c3ccc(-c4cccc5cccc(-c6ccccc6)c45)cc3)c3ccc4c(c3)sc3cc(C5CC6CCC5C6)ccc34)ccc21 ; c1ccc(-c2cccc3cccc(-c4ccc(N(c5ccc6c7ccc(C8CC9CCC8C9)cc7n(-c7ccccc7)c6c5)c5cccc6oc7ccccc7c56)cc4)c23)cc1]

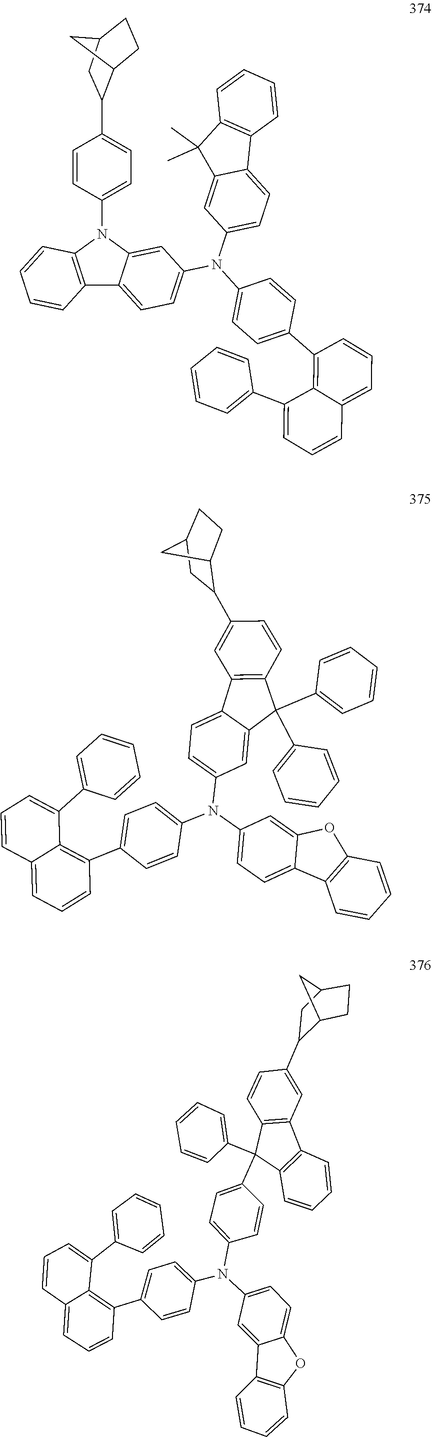 [SMILES: CC1(C)c2ccccc2-c2ccc(N(c3ccc(-c4cccc5cccc(-c6ccccc6)c45)cc3)c3ccc4c5ccccc5n(-c5ccc(C6CC7CCC6C7)cc5)c4c3)cc21 ; c1ccc(-c2cccc3cccc(-c4ccc(N(c5ccc6c(c5)C(c5ccccc5)(c5ccccc5)c5ccc(C7CC8CCC7C8)cc5-6)c5ccc6c(c5)oc5ccccc56)cc4)c23)cc1 ; c1ccc(-c2cccc3cccc(-c4ccc(N(c5ccc(C6(c7ccccc7)c7ccccc7-c7cc(C8CC9CCC8C9)ccc76)cc5)c5ccc6oc7ccccc7c6c5)cc4)c23)cc1]

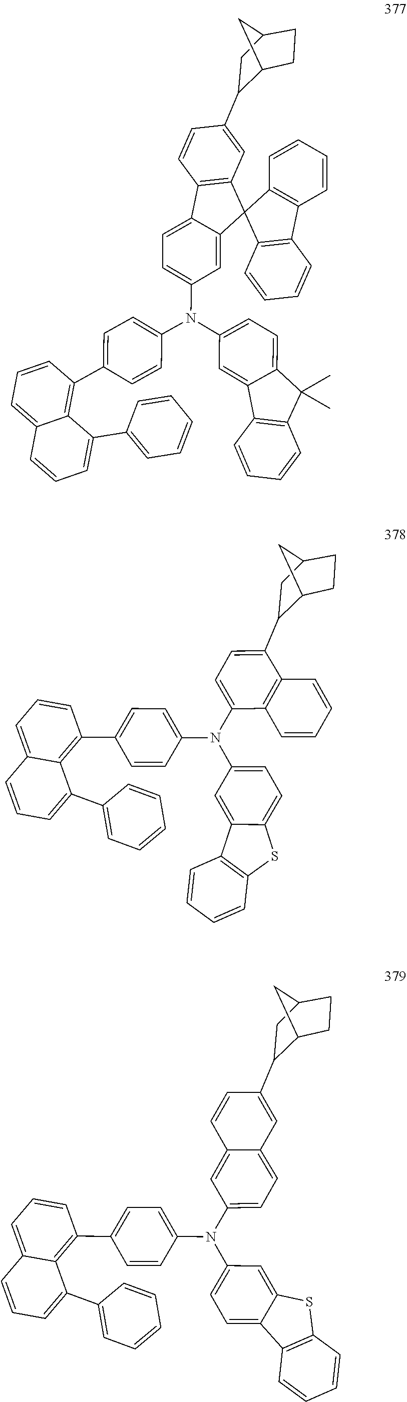 [SMILES: CC1(C)c2ccccc2-c2cc(N(c3ccc(-c4cccc5cccc(-c6ccccc6)c45)cc3)c3ccc4c(c3)C3(c5ccccc5-c5ccccc53)c3cc(C5CC6CCC5C6)ccc3-4)ccc21 ; c1ccc(-c2cccc3cccc(-c4ccc(N(c5ccc6sc7ccccc7c6c5)c5ccc(C6CC7CCC6C7)c6ccccc56)cc4)c23)cc1 ; c1ccc(-c2cccc3cccc(-c4ccc(N(c5ccc6cc(C7CC8CCC7C8)ccc6c5)c5ccc6c(c5)sc5ccccc56)cc4)c23)cc1]

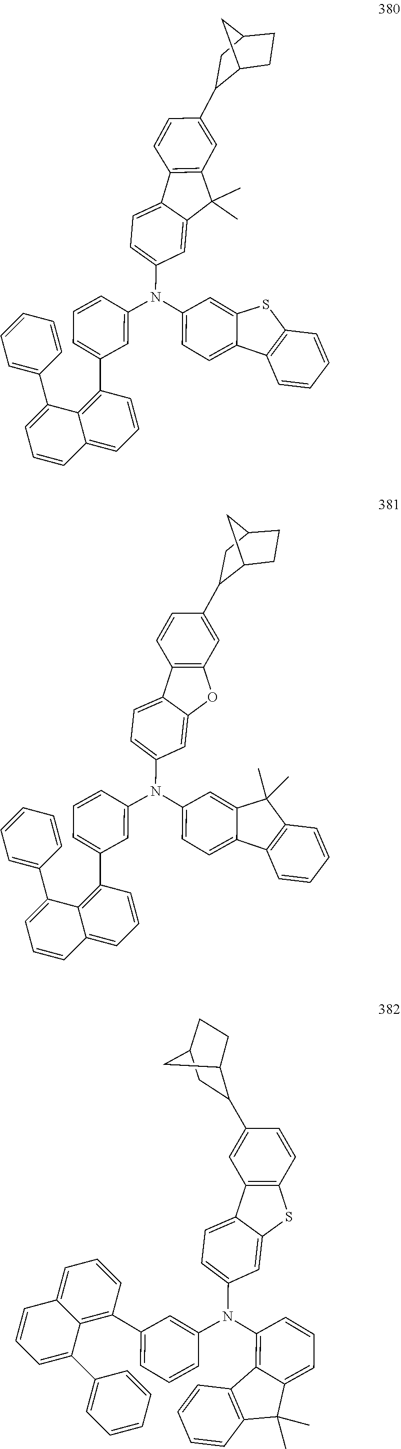 [SMILES: CC1(C)c2cc(C3CC4CCC3C4)ccc2-c2ccc(N(c3cccc(-c4cccc5cccc(-c6ccccc6)c45)c3)c3ccc4c(c3)sc3ccccc34)cc21 ; CC1(C)c2ccccc2-c2ccc(N(c3cccc(-c4cccc5cccc(-c6ccccc6)c45)c3)c3ccc4c(c3)oc3cc(C5CC6CCC5C6)ccc34)cc21 ; CC1(C)c2ccccc2-c2c(N(c3cccc(-c4cccc5cccc(-c6ccccc6)c45)c3)c3ccc4c(c3)sc3ccc(C5CC6CCC5C6)cc34)cccc21]

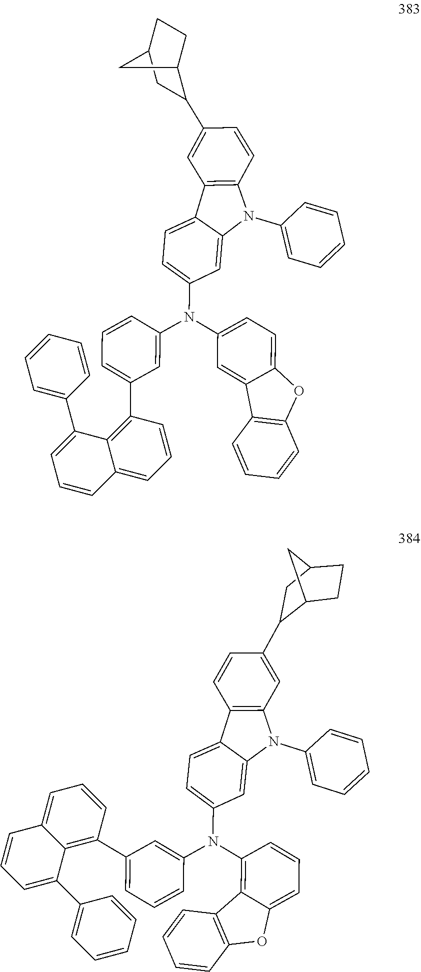 [SMILES: c1ccc(-c2cccc3cccc(-c4cccc(N(c5ccc6oc7ccccc7c6c5)c5ccc6c7cc(C8CC9CCC8C9)ccc7n(-c7ccccc7)c6c5)c4)c23)cc1 ; c1ccc(-c2cccc3cccc(-c4cccc(N(c5ccc6c7ccc(C8CC9CCC8C9)cc7n(-c7ccccc7)c6c5)c5cccc6oc7ccccc7c56)c4)c23)cc1]

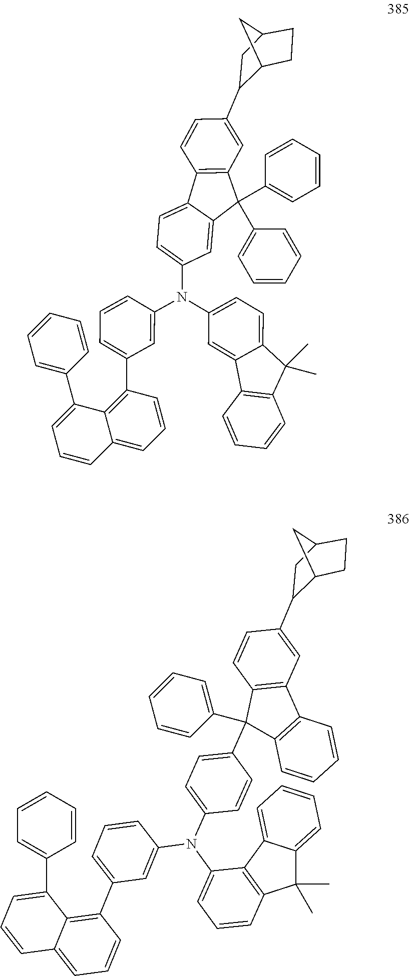 [SMILES: CC1(C)c2ccccc2-c2cc(N(c3cccc(-c4cccc5cccc(-c6ccccc6)c45)c3)c3ccc4c(c3)C(c3ccccc3)(c3ccccc3)c3cc(C5CC6CCC5C6)ccc3-4)ccc21 ; CC1(C)c2ccccc2-c2c(N(c3ccc(C4(c5ccccc5)c5ccccc5-c5cc(C6CC7CCC6C7)ccc54)cc3)c3cccc(-c4cccc5cccc(-c6ccccc6)c45)c3)cccc21]

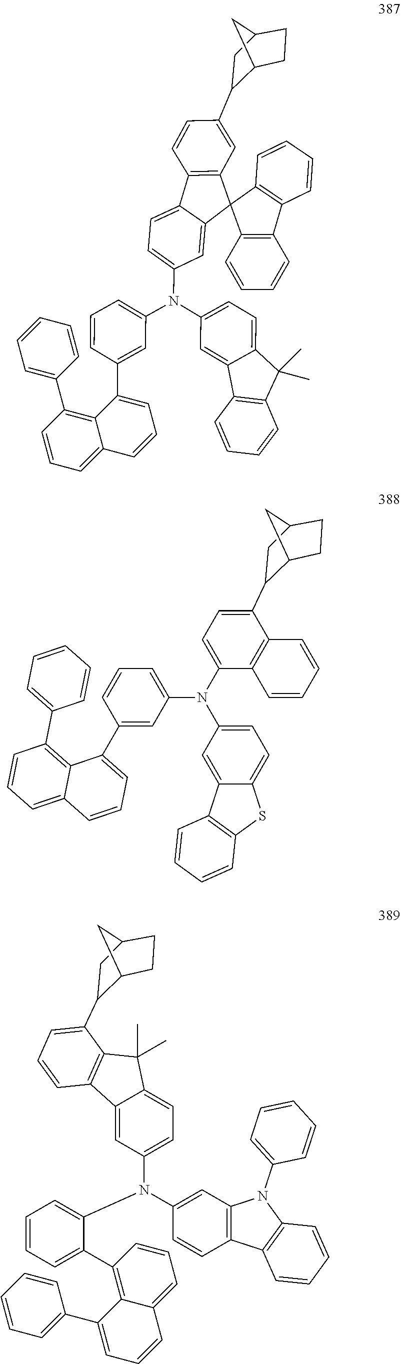 [SMILES: CC1(C)c2ccccc2-c2cc(N(c3cccc(-c4cccc5cccc(-c6ccccc6)c45)c3)c3ccc4c(c3)C3(c5ccccc5-c5ccccc53)c3cc(C5CC6CCC5C6)ccc3-4)ccc21 ; c1ccc(-c2cccc3cccc(-c4cccc(N(c5ccc6sc7ccccc7c6c5)c5ccc(C6CC7CCC6C7)c6ccccc56)c4)c23)cc1 ; CC1(C)c2ccc(N(c3ccc4c5ccccc5n(-c5ccccc5)c4c3)c3ccccc3-c3cccc4cccc(-c5ccccc5)c34)cc2-c2cccc(C3CC4CCC3C4)c21]

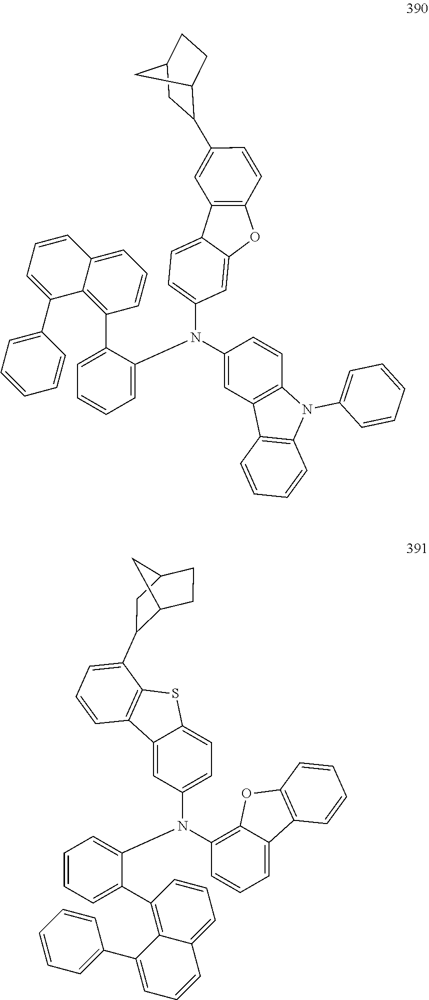 [SMILES: c1ccc(-c2cccc3cccc(-c4ccccc4N(c4ccc5c(c4)oc4ccc(C6CC7CCC6C7)cc45)c4ccc5c(c4)c4ccccc4n5-c4ccccc4)c23)cc1 ; c1ccc(-c2cccc3cccc(-c4ccccc4N(c4ccc5sc6c(C7CC8CCC7C8)cccc6c5c4)c4cccc5c4oc4ccccc45)c23)cc1]

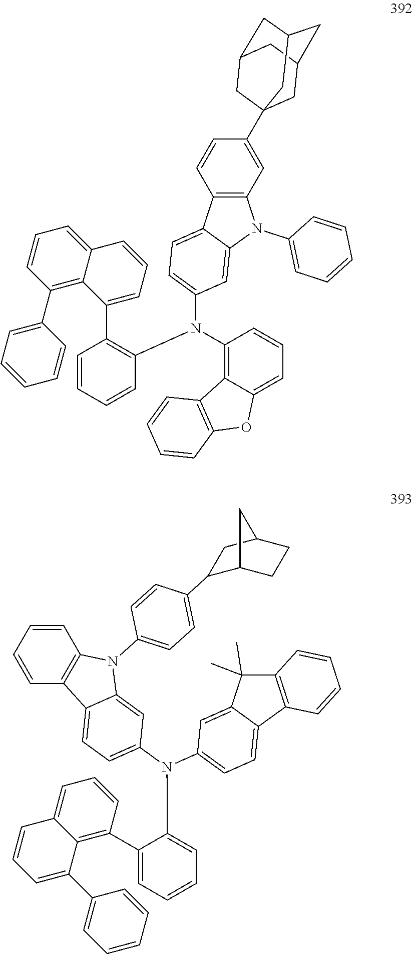 [SMILES: c1ccc(-c2cccc3cccc(-c4ccccc4N(c4ccc5c6ccc(C78CC9CC(CC(C9)C7)C8)cc6n(-c6ccccc6)c5c4)c4cccc5oc6ccccc6c45)c23)cc1 ; CC1(C)c2ccccc2-c2ccc(N(c3ccc4c5ccccc5n(-c5ccc(C6CC7CCC6C7)cc5)c4c3)c3ccccc3-c3cccc4cccc(-c5ccccc5)c34)cc21]

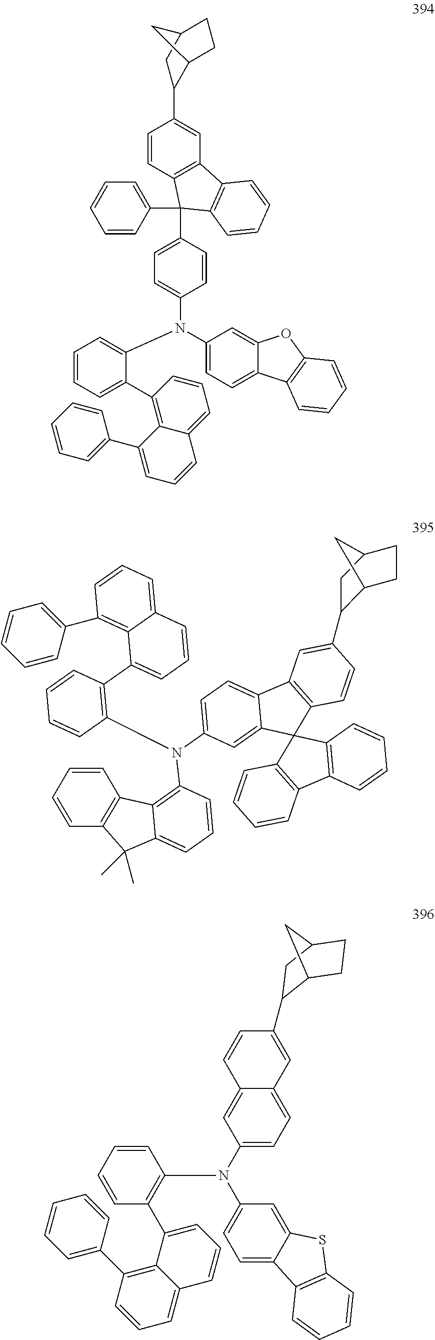 [SMILES: c1ccc(-c2cccc3cccc(-c4ccccc4N(c4ccc(C5(c6ccccc6)c6ccccc6-c6cc(C7CC8CCC7C8)ccc65)cc4)c4ccc5c(c4)oc4ccccc45)c23)cc1 ; CC1(C)c2ccccc2-c2c(N(c3ccc4c(c3)C3(c5ccccc5-c5ccccc53)c3ccc(C5CC6CCC5C6)cc3-4)c3ccccc3-c3cccc4cccc(-c5ccccc5)c34)cccc21 ; c1ccc(-c2cccc3cccc(-c4ccccc4N(c4ccc5cc(C6CC7CCC6C7)ccc5c4)c4ccc5c(c4)sc4ccccc45)c23)cc1]

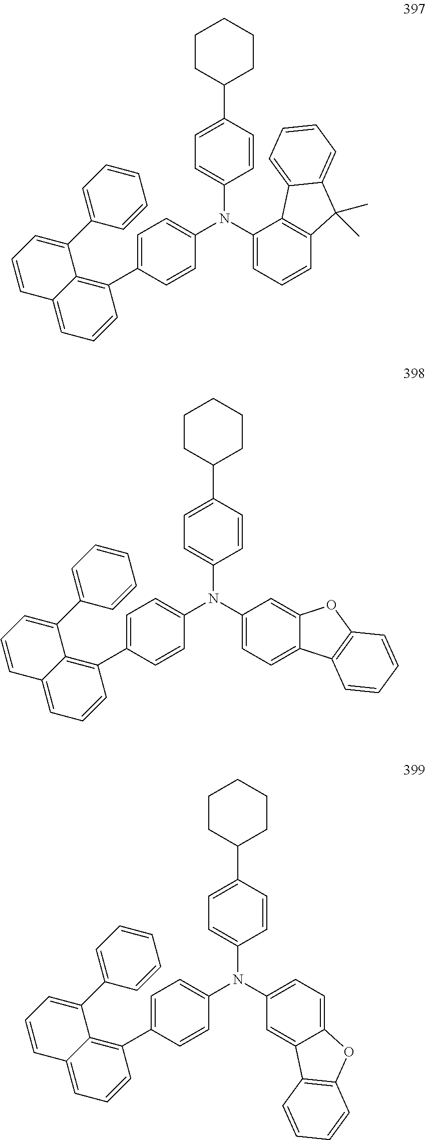 [SMILES: CC1(C)c2ccccc2-c2c(N(c3ccc(-c4cccc5cccc(-c6ccccc6)c45)cc3)c3ccc(C4CCCCC4)cc3)cccc21 ; c1ccc(-c2cccc3cccc(-c4ccc(N(c5ccc(C6CCCCC6)cc5)c5ccc6c(c5)oc5ccccc56)cc4)c23)cc1 ; c1ccc(-c2cccc3cccc(-c4ccc(N(c5ccc(C6CCCCC6)cc5)c5ccc6oc7ccccc7c6c5)cc4)c23)cc1]

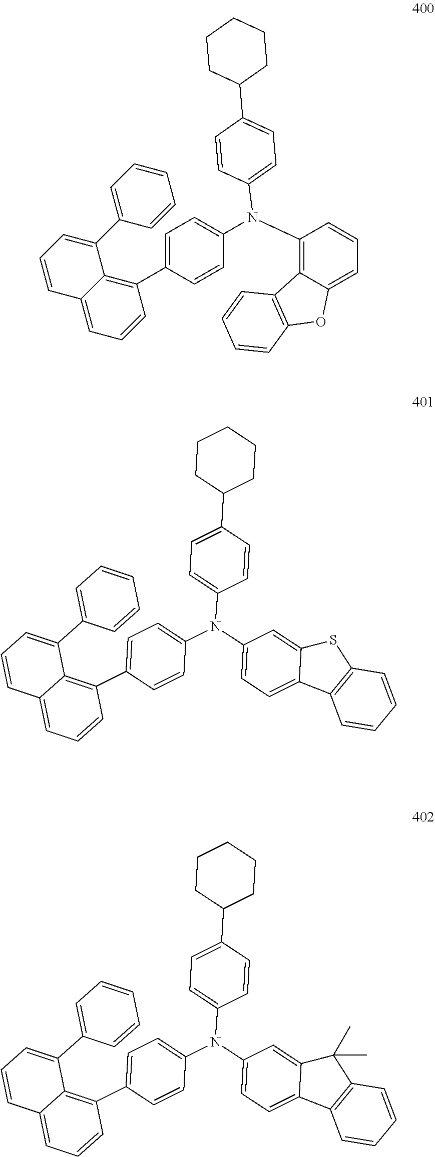 [SMILES: c1ccc(-c2cccc3cccc(-c4ccc(N(c5ccc(C6CCCCC6)cc5)c5cccc6oc7ccccc7c56)cc4)c23)cc1 ; c1ccc(-c2cccc3cccc(-c4ccc(N(c5ccc(C6CCCCC6)cc5)c5ccc6c(c5)sc5ccccc56)cc4)c23)cc1 ; CC1(C)c2ccccc2-c2ccc(N(c3ccc(-c4cccc5cccc(-c6ccccc6)c45)cc3)c3ccc(C4CCCCC4)cc3)cc21]

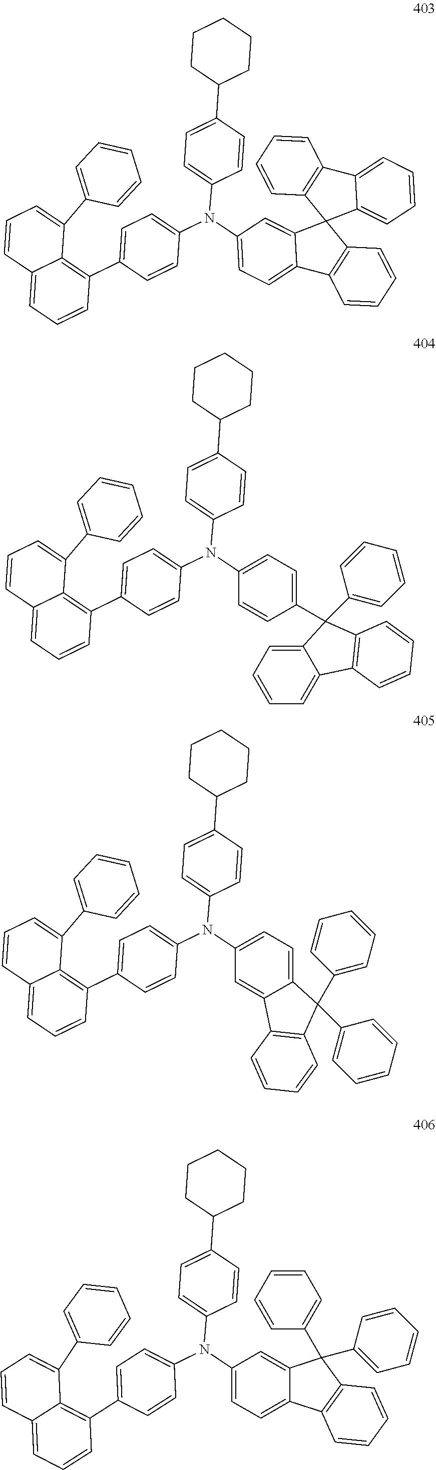 [SMILES: c1ccc(-c2cccc3cccc(-c4ccc(N(c5ccc(C6CCCCC6)cc5)c5ccc6c(c5)C5(c7ccccc7-c7ccccc75)c5ccccc5-6)cc4)c23)cc1 ; c1ccc(-c2cccc3cccc(-c4ccc(N(c5ccc(C6CCCCC6)cc5)c5ccc(C6(c7ccccc7)c7ccccc7-c7ccccc76)cc5)cc4)c23)cc1 ; c1ccc(-c2cccc3cccc(-c4ccc(N(c5ccc(C6CCCCC6)cc5)c5ccc6c(c5)-c5ccccc5C6(c5ccccc5)c5ccccc5)cc4)c23)cc1 ; c1ccc(-c2cccc3cccc(-c4ccc(N(c5ccc(C6CCCCC6)cc5)c5ccc6c(c5)C(c5ccccc5)(c5ccccc5)c5ccccc5-6)cc4)c23)cc1]

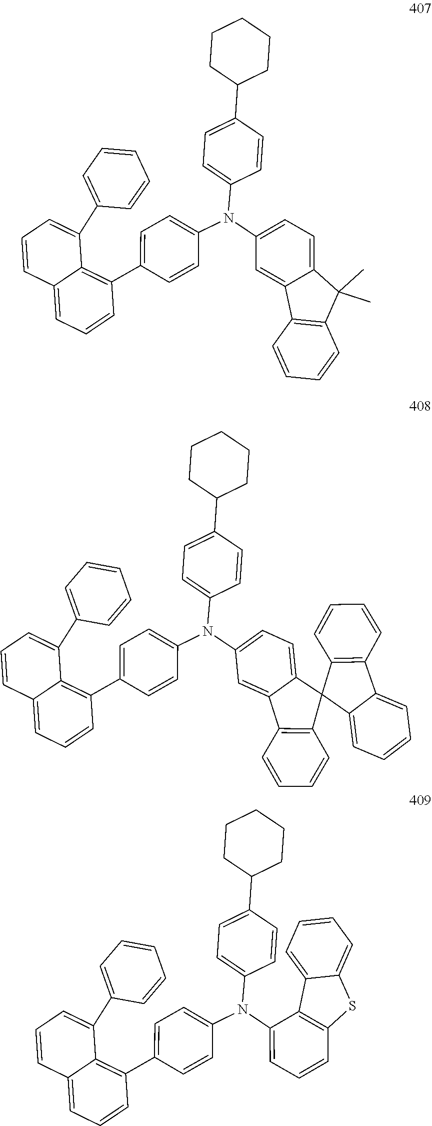

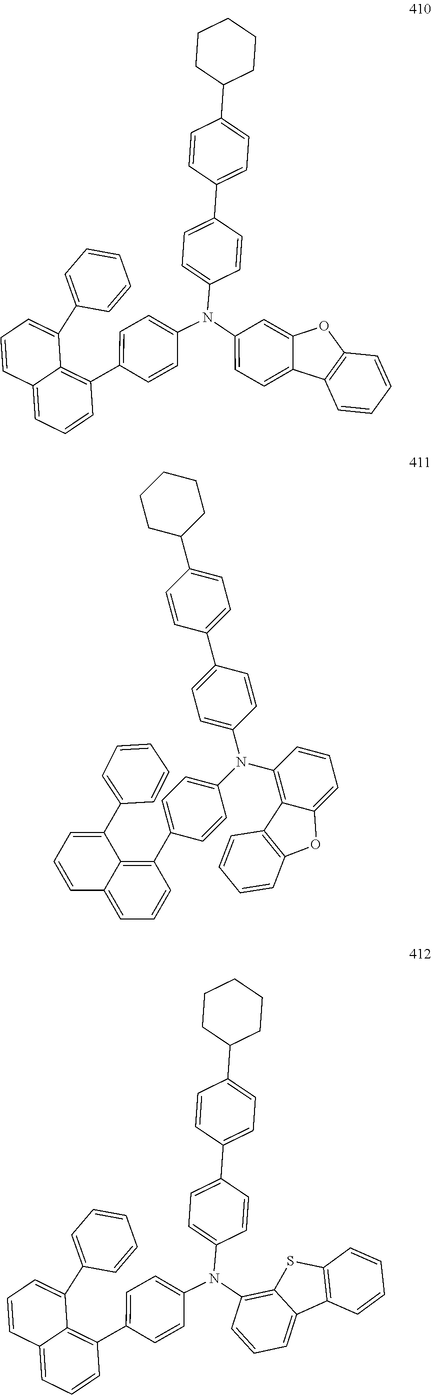 [SMILES: c1ccc(-c2cccc3cccc(-c4ccc(N(c5ccc(-c6ccc(C7CCCCC7)cc6)cc5)c5ccc6c(c5)oc5ccccc56)cc4)c23)cc1 ; c1ccc(-c2cccc3cccc(-c4ccc(N(c5ccc(-c6ccc(C7CCCCC7)cc6)cc5)c5cccc6oc7ccccc7c56)cc4)c23)cc1 ; c1ccc(-c2cccc3cccc(-c4ccc(N(c5ccc(-c6ccc(C7CCCCC7)cc6)cc5)c5cccc6c5sc5ccccc56)cc4)c23)cc1]

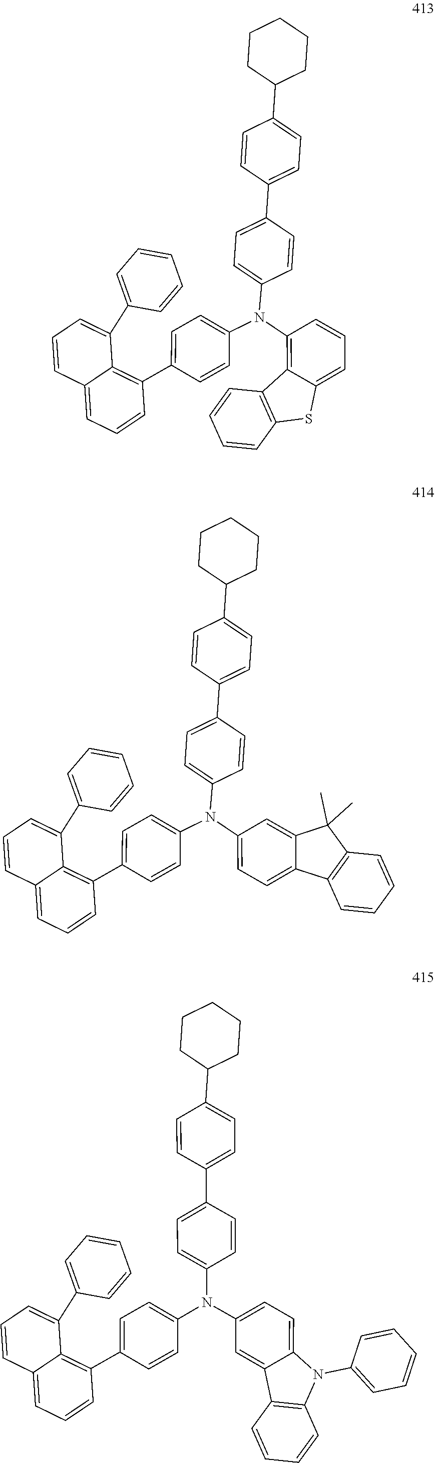 [SMILES: c1ccc(-c2cccc3cccc(-c4ccc(N(c5ccc(-c6ccc(C7CCCCC7)cc6)cc5)c5cccc6sc7ccccc7c56)cc4)c23)cc1 ; CC1(C)c2ccccc2-c2ccc(N(c3ccc(-c4ccc(C5CCCCC5)cc4)cc3)c3ccc(-c4cccc5cccc(-c6ccccc6)c45)cc3)cc21 ; c1ccc(-c2cccc3cccc(-c4ccc(N(c5ccc(-c6ccc(C7CCCCC7)cc6)cc5)c5ccc6c(c5)c5ccccc5n6-c5ccccc5)cc4)c23)cc1]

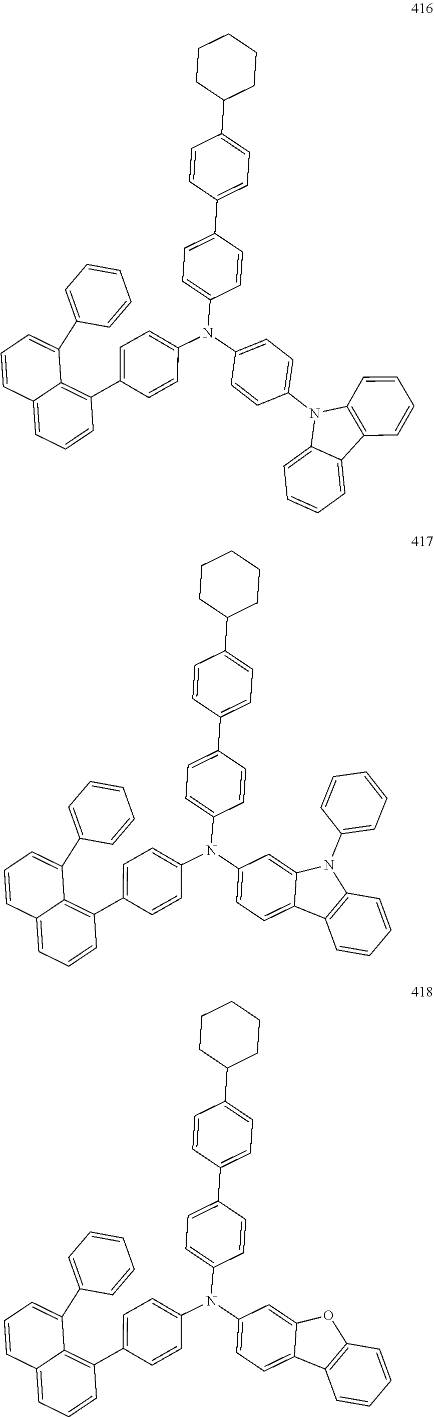 [SMILES: c1ccc(-c2cccc3cccc(-c4ccc(N(c5ccc(-c6ccc(C7CCCCC7)cc6)cc5)c5ccc(-n6c7ccccc7c7ccccc76)cc5)cc4)c23)cc1 ; c1ccc(-c2cccc3cccc(-c4ccc(N(c5ccc(-c6ccc(C7CCCCC7)cc6)cc5)c5ccc6c7ccccc7n(-c7ccccc7)c6c5)cc4)c23)cc1 ; c1ccc(-c2cccc3cccc(-c4ccc(N(c5ccc(-c6ccc(C7CCCCC7)cc6)cc5)c5ccc6c(c5)oc5ccccc56)cc4)c23)cc1]

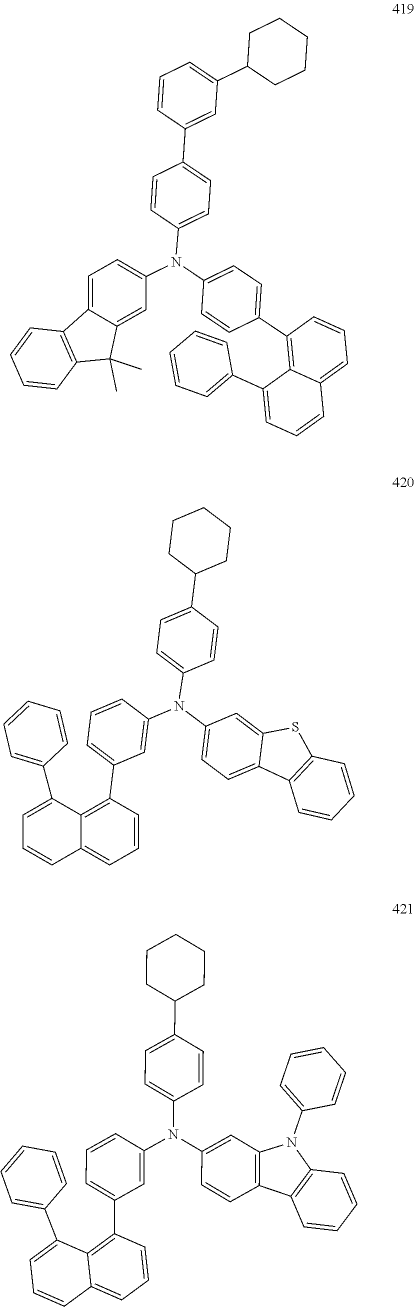 [SMILES: CC1(C)c2ccccc2-c2ccc(N(c3ccc(-c4cccc(C5CCCCC5)c4)cc3)c3ccc(-c4cccc5cccc(-c6ccccc6)c45)cc3)cc21 ; c1ccc(-c2cccc3cccc(-c4cccc(N(c5ccc(C6CCCCC6)cc5)c5ccc6c(c5)sc5ccccc56)c4)c23)cc1 ; c1ccc(-c2cccc3cccc(-c4cccc(N(c5ccc(C6CCCCC6)cc5)c5ccc6c7ccccc7n(-c7ccccc7)c6c5)c4)c23)cc1]

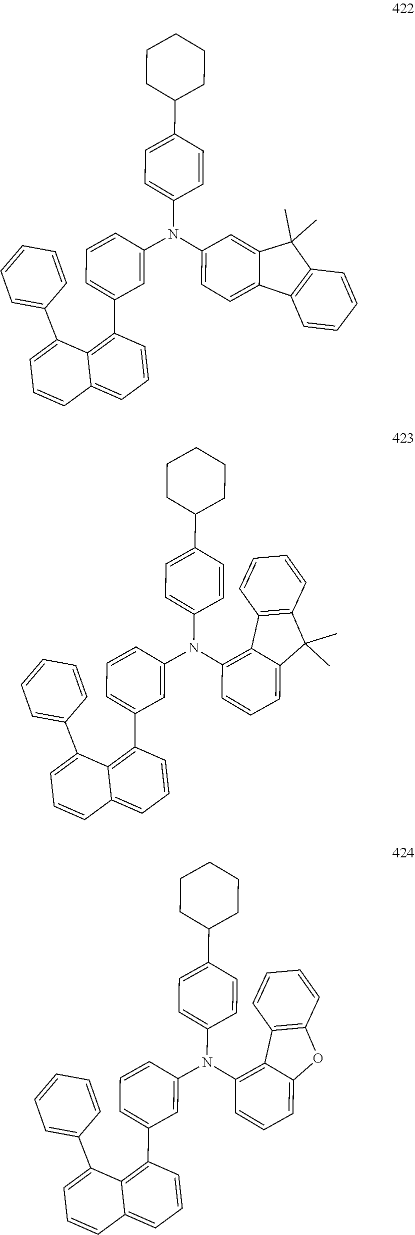 [SMILES: CC1(C)c2ccccc2-c2ccc(N(c3ccc(C4CCCCC4)cc3)c3cccc(-c4cccc5cccc(-c6ccccc6)c45)c3)cc21 ; CC1(C)c2ccccc2-c2c(N(c3ccc(C4CCCCC4)cc3)c3cccc(-c4cccc5cccc(-c6ccccc6)c45)c3)cccc21 ; c1ccc(-c2cccc3cccc(-c4cccc(N(c5ccc(C6CCCCC6)cc5)c5cccc6oc7ccccc7c56)c4)c23)cc1]

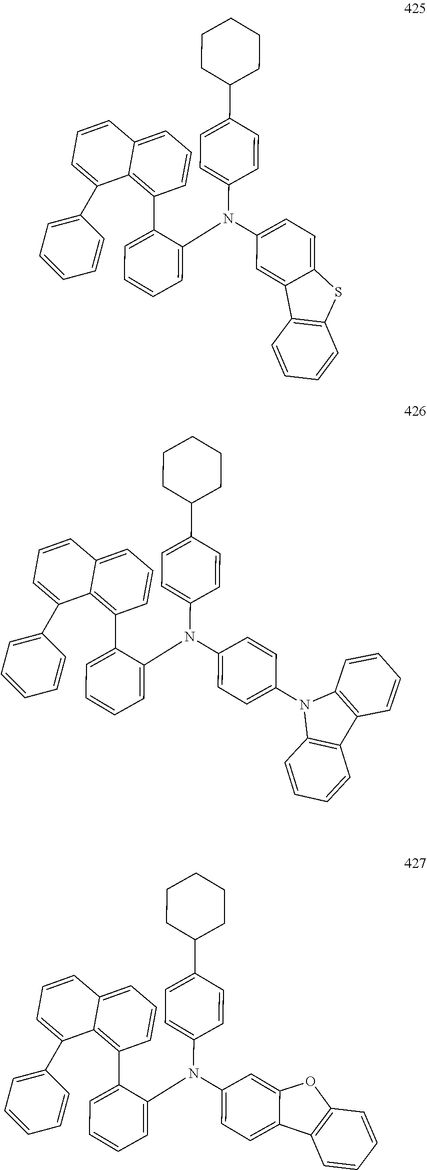 [SMILES: c1ccc(-c2cccc3cccc(-c4ccccc4N(c4ccc(C5CCCCC5)cc4)c4ccc5sc6ccccc6c5c4)c23)cc1 ; c1ccc(-c2cccc3cccc(-c4ccccc4N(c4ccc(C5CCCCC5)cc4)c4ccc(-n5c6ccccc6c6ccccc65)cc4)c23)cc1 ; c1ccc(-c2cccc3cccc(-c4ccccc4N(c4ccc(C5CCCCC5)cc4)c4ccc5c(c4)oc4ccccc45)c23)cc1]

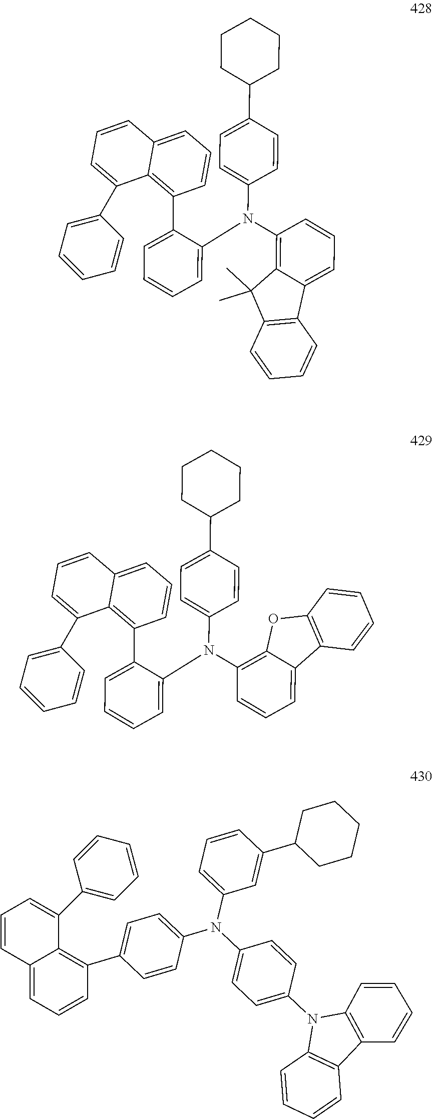 [SMILES: CC1(C)c2ccccc2-c2cccc(N(c3ccc(C4CCCCC4)cc3)c3ccccc3-c3cccc4cccc(-c5ccccc5)c34)c21 ; c1ccc(-c2cccc3cccc(-c4ccccc4N(c4ccc(C5CCCCC5)cc4)c4cccc5c4oc4ccccc45)c23)cc1 ; c1ccc(-c2cccc3cccc(-c4ccc(N(c5ccc(-n6c7ccccc7c7ccccc76)cc5)c5cccc(C6CCCCC6)c5)cc4)c23)cc1]

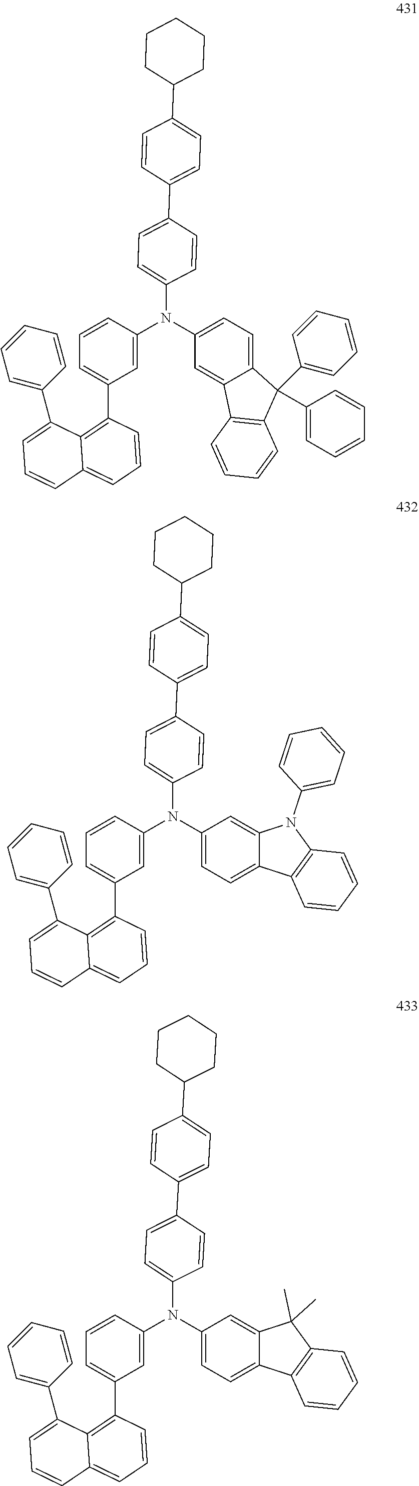 [SMILES: c1ccc(-c2cccc3cccc(-c4cccc(N(c5ccc(-c6ccc(C7CCCCC7)cc6)cc5)c5ccc6c(c5)-c5ccccc5C6(c5ccccc5)c5ccccc5)c4)c23)cc1 ; c1ccc(-c2cccc3cccc(-c4cccc(N(c5ccc(-c6ccc(C7CCCCC7)cc6)cc5)c5ccc6c7ccccc7n(-c7ccccc7)c6c5)c4)c23)cc1 ; CC1(C)c2ccccc2-c2ccc(N(c3ccc(-c4ccc(C5CCCCC5)cc4)cc3)c3cccc(-c4cccc5cccc(-c6ccccc6)c45)c3)cc21]

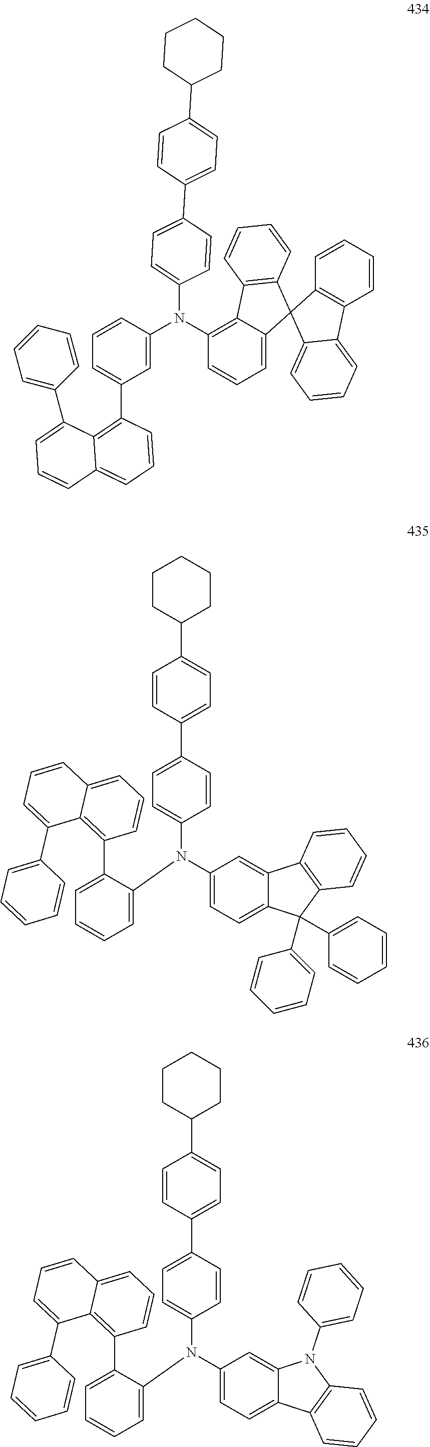 [SMILES: c1ccc(-c2cccc3cccc(-c4cccc(N(c5ccc(-c6ccc(C7CCCCC7)cc6)cc5)c5cccc6c5-c5ccccc5C65c6ccccc6-c6ccccc65)c4)c23)cc1 ; c1ccc(-c2cccc3cccc(-c4ccccc4N(c4ccc(-c5ccc(C6CCCCC6)cc5)cc4)c4ccc5c(c4)-c4ccccc4C5(c4ccccc4)c4ccccc4)c23)cc1 ; c1ccc(-c2cccc3cccc(-c4ccccc4N(c4ccc(-c5ccc(C6CCCCC6)cc5)cc4)c4ccc5c6ccccc6n(-c6ccccc6)c5c4)c23)cc1]

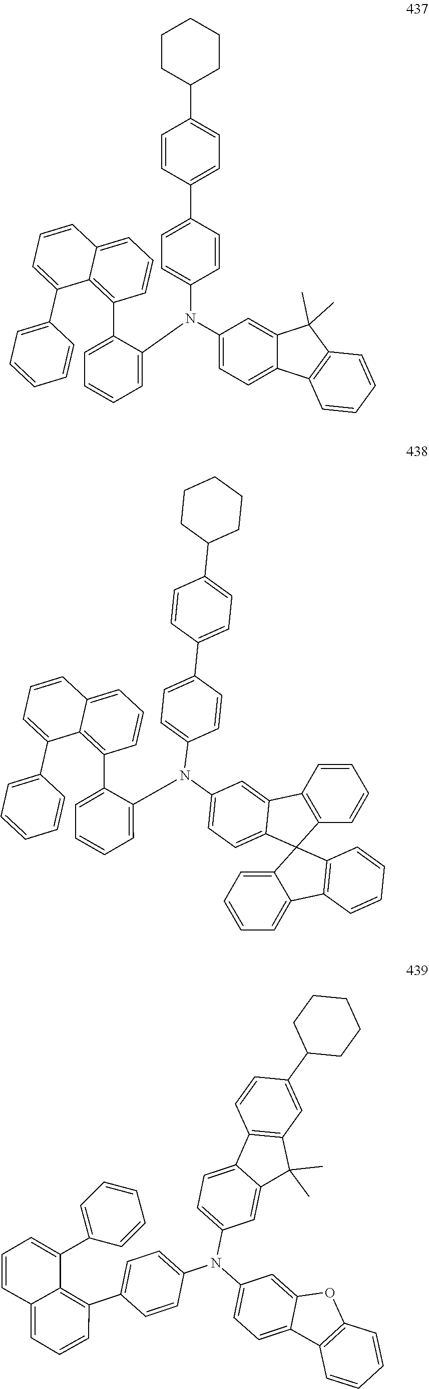 [SMILES: CC1(C)c2ccccc2-c2ccc(N(c3ccc(-c4ccc(C5CCCCC5)cc4)cc3)c3ccccc3-c3cccc4cccc(-c5ccccc5)c34)cc21 ; c1ccc(-c2cccc3cccc(-c4ccccc4N(c4ccc(-c5ccc(C6CCCCC6)cc5)cc4)c4ccc5c(c4)-c4ccccc4C54c5ccccc5-c5ccccc54)c23)cc1 ; CC1(C)c2cc(C3CCCCC3)ccc2-c2ccc(N(c3ccc(-c4cccc5cccc(-c6ccccc6)c45)cc3)c3ccc4c(c3)oc3ccccc34)cc21]

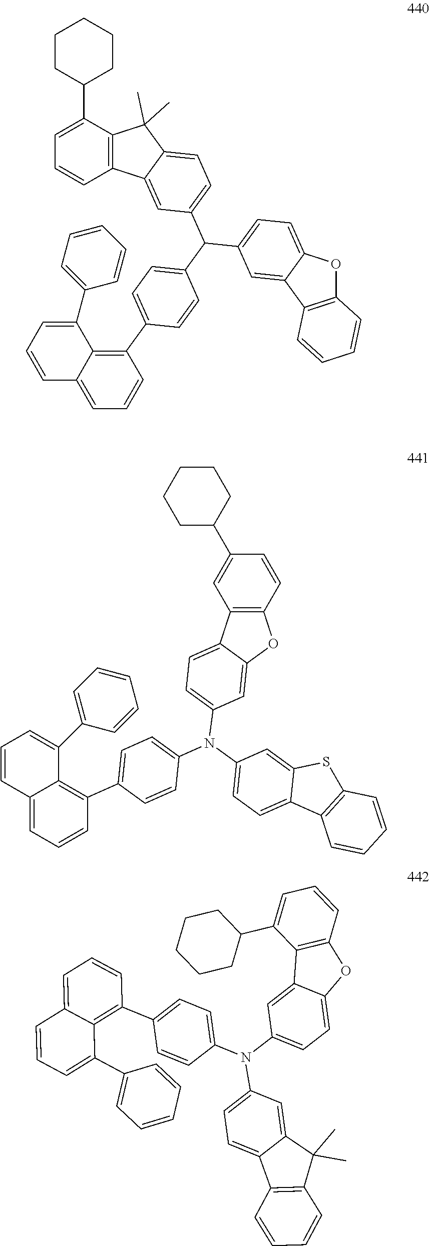

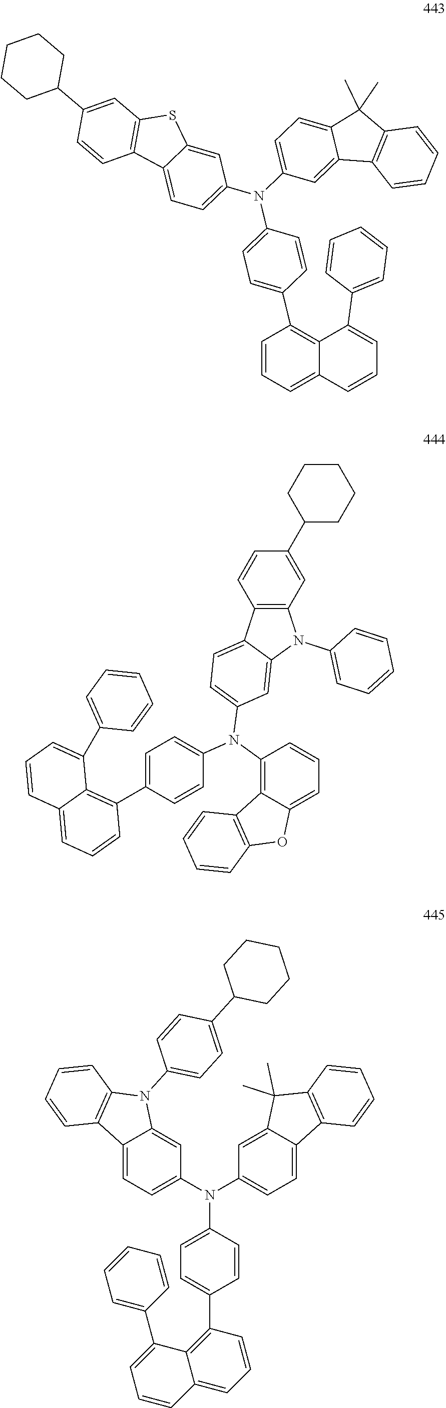 [SMILES: CC1(C)c2ccccc2-c2cc(N(c3ccc(-c4cccc5cccc(-c6ccccc6)c45)cc3)c3ccc4c(c3)sc3cc(C5CCCCC5)ccc34)ccc21 ; c1ccc(-c2cccc3cccc(-c4ccc(N(c5ccc6c7ccc(C8CCCCC8)cc7n(-c7ccccc7)c6c5)c5cccc6oc7ccccc7c56)cc4)c23)cc1 ; CC1(C)c2ccccc2-c2ccc(N(c3ccc(-c4cccc5cccc(-c6ccccc6)c45)cc3)c3ccc4c5ccccc5n(-c5ccc(C6CCCCC6)cc5)c4c3)cc21]

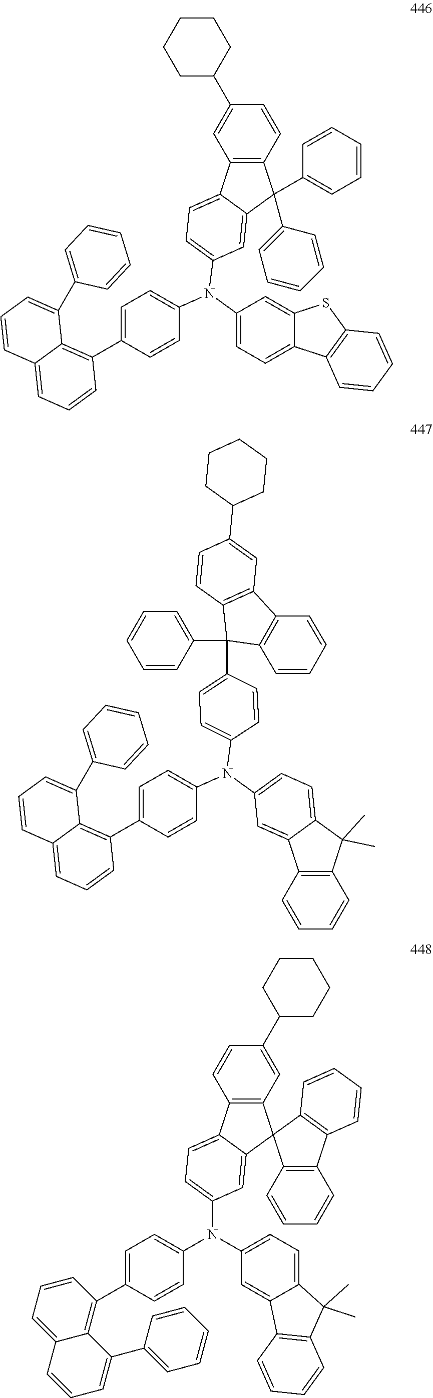

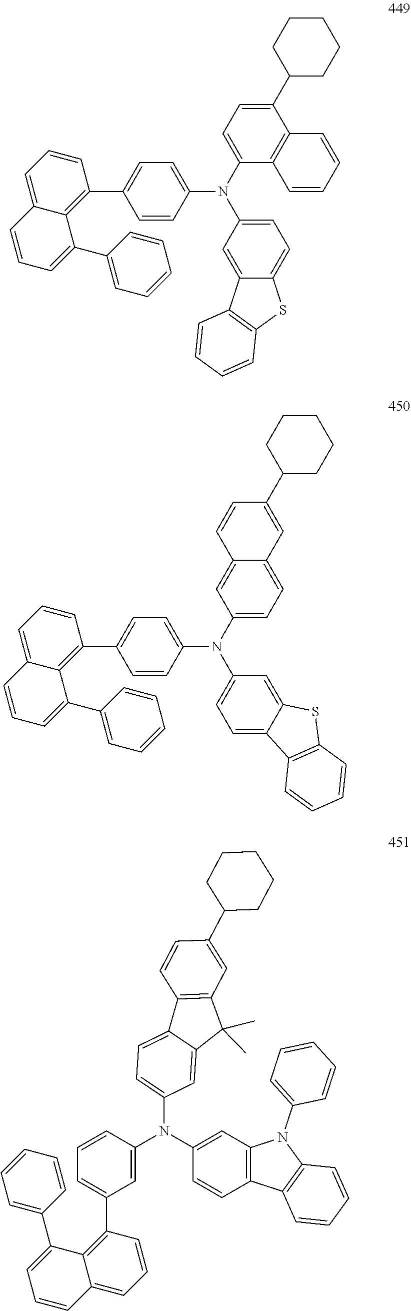 [SMILES: c1ccc(-c2cccc3cccc(-c4ccc(N(c5ccc6sc7ccccc7c6c5)c5ccc(C6CCCCC6)c6ccccc56)cc4)c23)cc1 ; c1ccc(-c2cccc3cccc(-c4ccc(N(c5ccc6cc(C7CCCCC7)ccc6c5)c5ccc6c(c5)sc5ccccc56)cc4)c23)cc1 ; CC1(C)c2cc(C3CCCCC3)ccc2-c2ccc(N(c3cccc(-c4cccc5cccc(-c6ccccc6)c45)c3)c3ccc4c5ccccc5n(-c5ccccc5)c4c3)cc21]

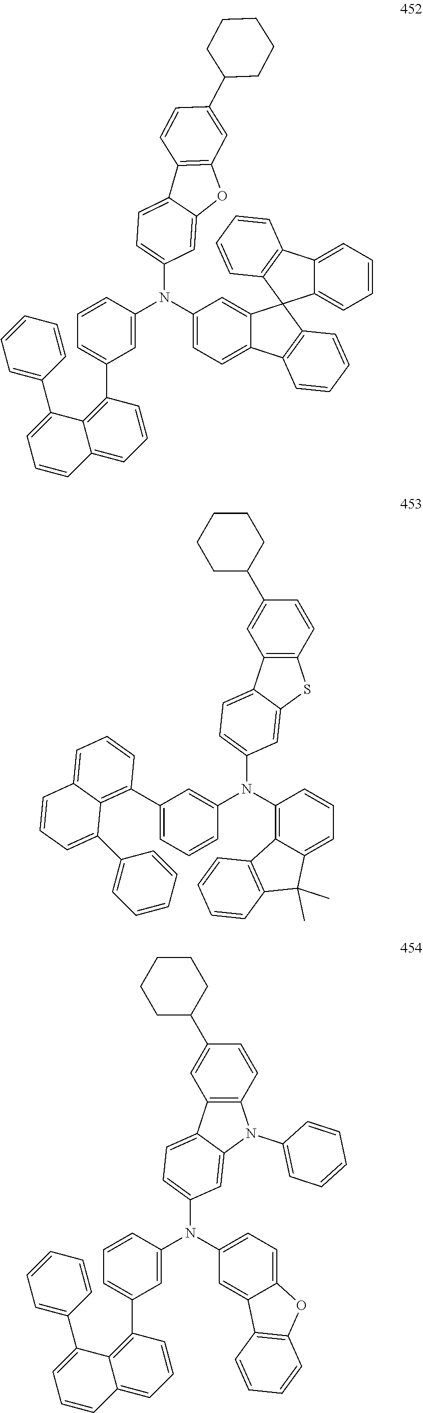 [SMILES: c1ccc(-c2cccc3cccc(-c4cccc(N(c5ccc6c(c5)C5(c7ccccc7-c7ccccc75)c5ccccc5-6)c5ccc6c(c5)oc5cc(C7CCCCC7)ccc56)c4)c23)cc1 ; CC1(C)c2ccccc2-c2c(N(c3cccc(-c4cccc5cccc(-c6ccccc6)c45)c3)c3ccc4c(c3)sc3ccc(C5CCCCC5)cc34)cccc21 ; c1ccc(-c2cccc3cccc(-c4cccc(N(c5ccc6oc7ccccc7c6c5)c5ccc6c7cc(C8CCCCC8)ccc7n(-c7ccccc7)c6c5)c4)c23)cc1]

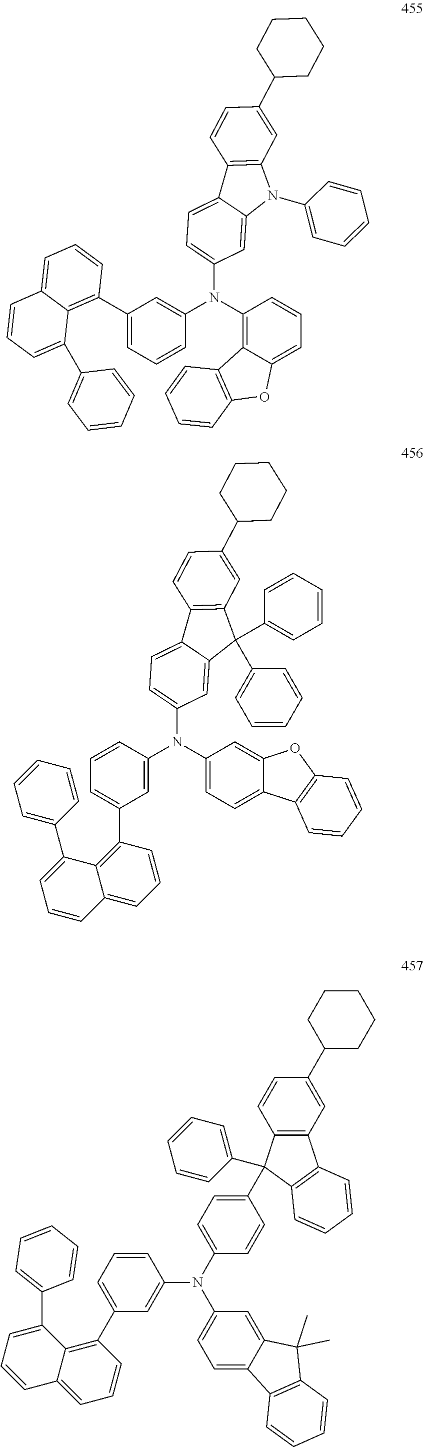

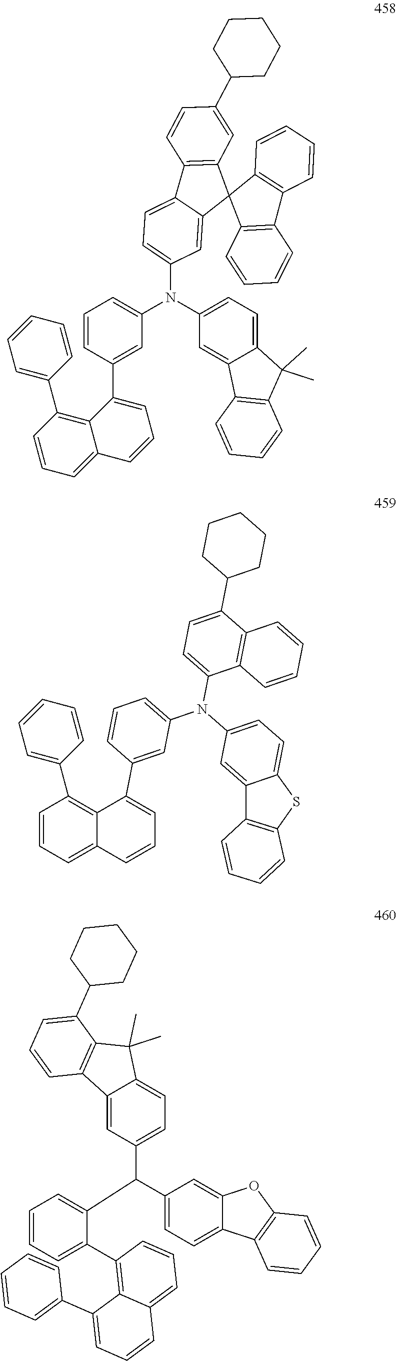 [SMILES: CC1(C)c2ccccc2-c2cc(N(c3cccc(-c4cccc5cccc(-c6ccccc6)c45)c3)c3ccc4c(c3)C3(c5ccccc5-c5ccccc53)c3cc(C5CCCCC5)ccc3-4)ccc21 ; c1ccc(-c2cccc3cccc(-c4cccc(N(c5ccc6sc7ccccc7c6c5)c5ccc(C6CCCCC6)c6ccccc56)c4)c23)cc1 ; CC1(C)c2ccc(C(c3ccc4c(c3)oc3ccccc34)c3ccccc3-c3cccc4cccc(-c5ccccc5)c34)cc2-c2cccc(C3CCCCC3)c21]

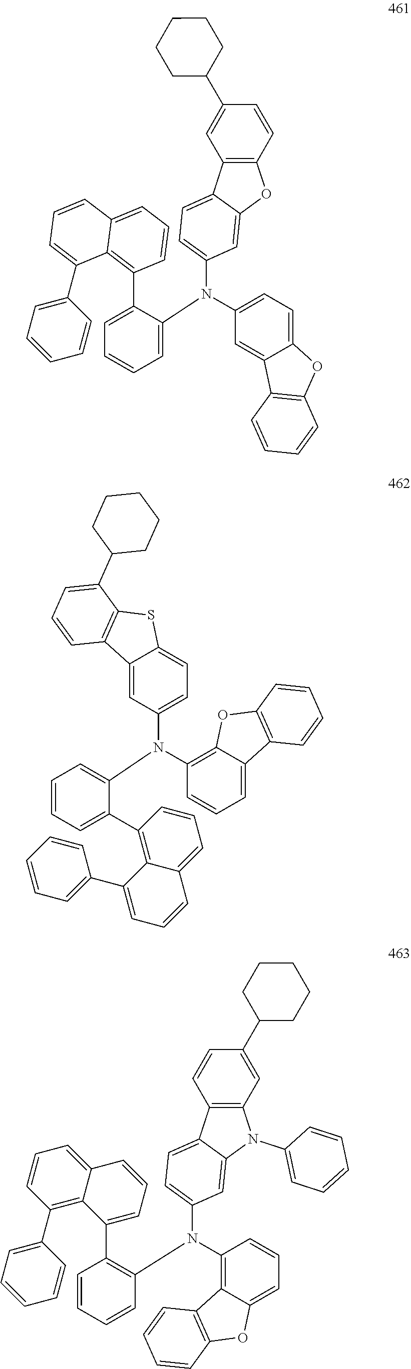 [SMILES: c1ccc(-c2cccc3cccc(-c4ccccc4N(c4ccc5c(c4)oc4ccc(C6CCCCC6)cc45)c4ccc5oc6ccccc6c5c4)c23)cc1 ; c1ccc(-c2cccc3cccc(-c4ccccc4N(c4ccc5sc6c(C7CCCCC7)cccc6c5c4)c4cccc5c4oc4ccccc45)c23)cc1 ; c1ccc(-c2cccc3cccc(-c4ccccc4N(c4ccc5c6ccc(C7CCCCC7)cc6n(-c6ccccc6)c5c4)c4cccc5oc6ccccc6c45)c23)cc1]

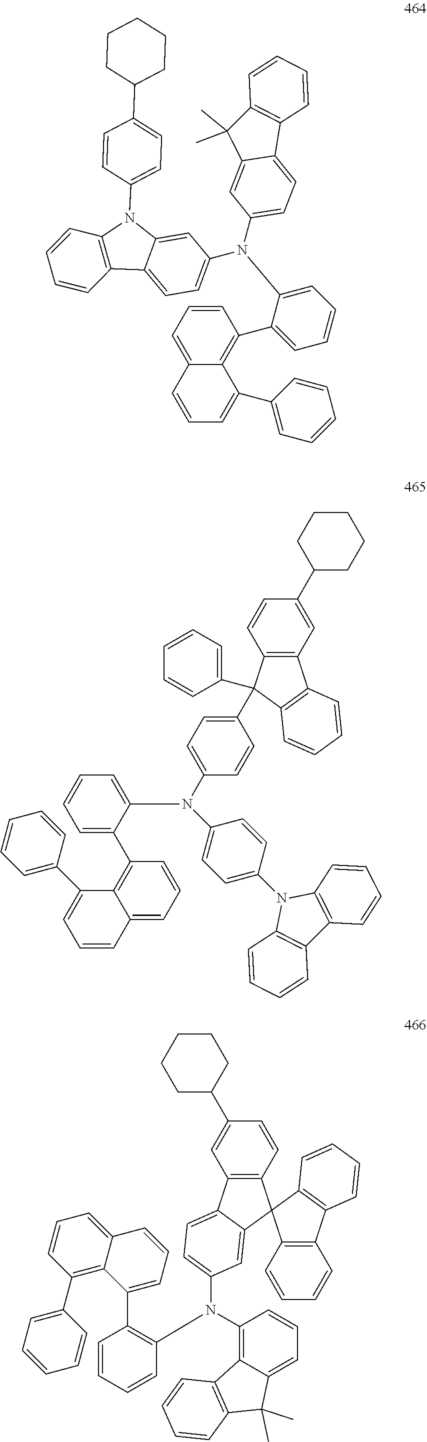 [SMILES: CC1(C)c2ccccc2-c2ccc(N(c3ccc4c5ccccc5n(-c5ccc(C6CCCCC6)cc5)c4c3)c3ccccc3-c3cccc4cccc(-c5ccccc5)c34)cc21 ; c1ccc(-c2cccc3cccc(-c4ccccc4N(c4ccc(-n5c6ccccc6c6ccccc65)cc4)c4ccc(C5(c6ccccc6)c6ccccc6-c6cc(C7CCCCC7)ccc65)cc4)c23)cc1 ; CC1(C)c2ccccc2-c2c(N(c3ccc4c(c3)C3(c5ccccc5-c5ccccc53)c3ccc(C5CCCCC5)cc3-4)c3ccccc3-c3cccc4cccc(-c5ccccc5)c34)cccc21]

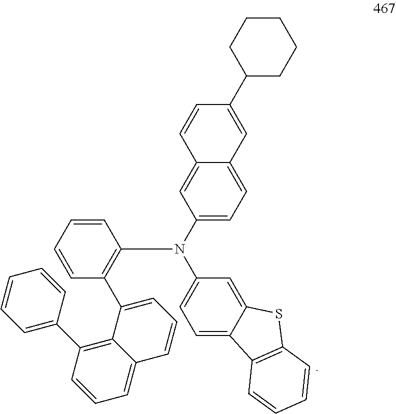 [SMILES: c1ccc(-c2cccc3cccc(-c4ccccc4N(c4ccc5cc(C6CCCCC6)ccc5c4)c4ccc5c(c4)sc4ccccc45)c23)cc1]

In a second aspect, the present disclosure provides an electronic component, including an anode and a cathode which are oppositely disposed, and a functional layer disposed between the anode and the cathode; and the functional layer includes the organic compound of the present disclosure.

In one specific example, the functional layer includes an electron blocking layer including the organic compound. Optionally, the electronic component is an organic electroluminescent device.

Optionally, the organic electroluminescent device is a blue light device or a green light device.

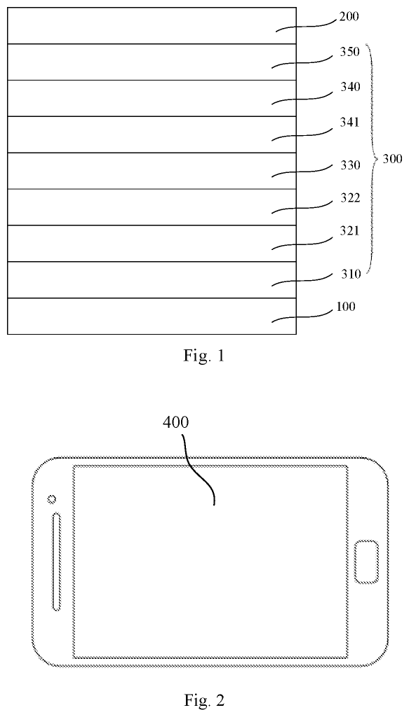

In one specific example, as shown in FIG. 1, the organic electroluminescent device may include an anode 100, a hole transport layer 321, an electron blocking layer 322, an organic light-emitting layer 330, an electron transport layer 340, and a cathode 200 which are sequentially stacked.

Optionally, the anode 100 includes the following anode materials, which are preferably materials having a large work function that facilitate hole injection into the functional layer. Specific examples of the anode materials include metals such as nickel, platinum, vanadium, chromium, copper, zinc, and gold or their alloys; metal oxides such as zinc oxide, indium oxide, indium tin oxide (ITO) and indium zinc oxide (IZO); combined metals and oxides such as ZnO:Al or SnO₂:Sb; or conducting polymers such as poly(3-methylthiophene), poly[3,4-(ethylene-1,2-dioxy)thiophene] (PEDT), polypyrrole and polyaniline, but are not limited to this. A transparent electrode containing indium tin oxide (ITO) as the anode is preferably included.

In the present disclosure, the hole transport layer 321 may be made of a carbazole polymer, carbazole-linked triarylamine compounds, or other types of compounds, which is not particularly limited in the present disclosure. For example, the hole transport layer 321 may be NPB.

In the present disclosure, the electron blocking layer 322 may be composed of the organic compound of the present disclosure or may be composed of the organic compound provided by the present disclosure together with other materials, and the other materials may be selected from a carbazole polymer, carbazole-linked triarylamine compounds or other compounds conventionally employed by those skilled in the art in the electron blocking layer. For example, the electron blocking layer may be the organic compound of the present disclosure.

In the present disclosure, the organic light-emitting layer 330 may consist of a single light-emitting material or may include a host material and a dopant material. Optionally, the organic light-emitting layer 330 is composed of the host material and the dopant material, and holes injected into the organic light-emitting layer 330 and electrons injected into the organic light-emitting layer 330 can be recombined in the organic light-emitting layer 330 to form excitons, the excitons transfer energy to the host material, and the host material transfers energy to the dopant material, thus enabling the dopant material to emit light.

The host material of the organic light-emitting layer 330 can be a metal chelate compound, a bis-styryl derivative, an aromatic amine derivative, a dibenzofuran derivative or other types of materials, which is not specially limited in the present disclosure. In one example of the present disclosure, the host material of the organic light-emitting layer 330 is BH-01.

The dopant material of the organic light-emitting layer 330 may be a compound having a condensed aryl ring or its derivative, a compound having a heteroaryl ring or its derivative, an aromatic amine derivative, or other materials, which is not specially limited in the present disclosure. In one example of the present disclosure, the dopant material of the organic light-emitting layer 330 is BD-01.

The electron transport layer 340 may be a single-layer structure or a multi-layer structure, and may include one or more electron transport materials, and the electron transport materials may be selected from, but are not limited to, a benzimidazole derivative, an oxadiazole derivative, a quinoxaline derivative, or other electron transport materials. In one example of the present disclosure, the electron transport layer 340 consists of ET-06 and LiQ.

In the present disclosure, the cathode 200 includes a cathode material, which is a material having a small work function that facilitates electron injection into the functional layer. Specific examples of the cathode material include metals such as magnesium, calcium, sodium, potassium, titanium, indium, yttrium, lithium, gadolinium, aluminum, silver, tin, and lead or their alloys; or multilayer materials such as LiF/Al, Liq/Al, LiO₂/Al, LiF/Ca, LiF/Al, and BaF₂/Ca, but are not limited to this. A metal electrode containing magnesium and silver as the cathode is preferably included.

Optionally, as shown in FIG. 1, a hole injection layer 310 is also arranged between the anode 100 and the hole transport layer 321 to enhance the ability to inject holes into the hole transport layer 321. The hole injection layer 310 can be made of a benzidine derivative, a starburst arylamine compound, a phthalocyanine derivative or other materials, which is not specially limited in the present disclosure. For example, a material of the hole injection layer 310 is F4-TCNQ.

Optionally, as shown in FIG. 1, an electron injection layer 350 is also arranged between the cathode 200 and the electron transport layer 340 to enhance the ability to inject electrons into the electron transport layer 340. The electron injection layer 350 may include an inorganic material such as an alkali metal sulfide and an alkali metal halide, or may include a complex of an alkali metal and an organic substance. For example, a material of the electron injection layer 350 is Yb.

Optionally, as shown in FIG. 1, a hole blocking layer 341 may or may not be disposed between the organic light-emitting layer 330 and the electron transport layer 340, and a material of the hole blocking layer 341 is well known in the art, which will not be repeated here.

In a third aspect, the present disclosure provides an electronic device, including the electronic component according to the second aspect of the present disclosure.

According to one example, as shown in FIG. 2, the electronic device is an electronic device 400 including the organic electroluminescent device described above. The electronic device 400 may be, for example, a display device, a lighting device, an optical communication device, or other types of electronic devices, and may include, for example but is not limited to, a computer screen, a mobile phone screen, a television, electronic paper, an emergency lighting lamp, an optical module, and the like.

The synthesis methods of the organic compounds of the present disclosure are specifically described below in conjunction with synthesis examples, but the present disclosure is not limited in any way.

Compounds of which synthetic methods are not mentioned in the present disclosure are raw material products obtained by commercial routes.

### SYNTHESIS EXAMPLES

### Synthesis of IMA-X

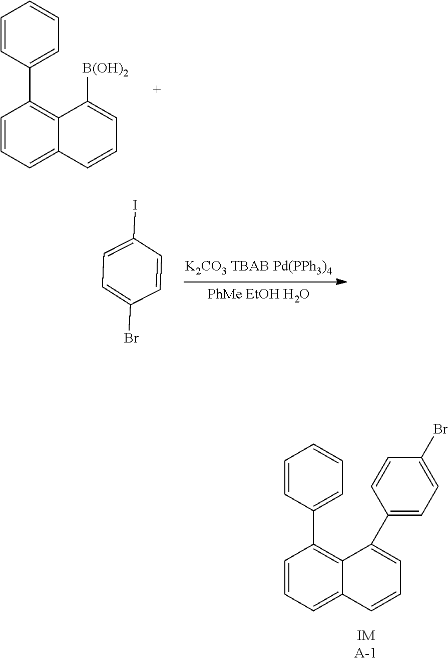 [SMILES: OB(O)c1cccc2cccc(-c3ccccc3)c12 ; Brc1ccc(I)cc1 ; Brc1ccc(-c2cccc3cccc(-c4ccccc4)c23)cc1]

Under the protection of N₂, 8-phenyl-1-naphthaleneboronic acid (100 g, 0.403 mol), p-bromoiodobenzene (103.7 g, 0.366 mol), potassium carbonate (K₂CO₃) (101.3 g, 0.733 mol), tetrabutylammonium bromide (TBAB) (2.3 g, 0.007 mol), tetrakis(triphenylphosphine)palladium (Pd(PPh₃)₄) (4.2 g, 0.004 mol), toluene (PhMe) (600 mL), ethanol (EtOH) (200 mL), and water (H₂O) (100 mL) were added to a 1000 mL three-necked flask, after a reaction was carried out under stirring and reflux at 80° C. for 12 h, the reaction was stopped, after the reaction solution was cooled to room temperature, the reaction solution was extracted with deionized water/toluene, an organic phase was washed with water to be neutral, anhydrous magnesium sulfate was added to remove water, filtering was performed, and the obtained filtrate was concentrated, and allowed to pass through a chromatographic column using an eluent dichloromethane:n-heptane (v/v)=1:5 to give IM A-1 (117.9 g, yield: 90%) as a white solid.

IM A-X listed in Table 1 was synthesized with reference to the method for IMA-1, except that a raw material 1 was used instead of p-bromoiodobenzene. The used main raw materials, the synthesized intermediates and their yields are shown in Table 1.

TABLE 1

| Raw | | |
|---|---|---|
| material | | Yield/ |
| 1 | IM A-X | % |
| 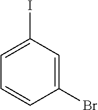 | 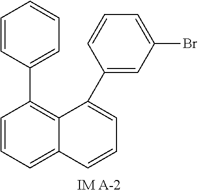 | 89 |
| 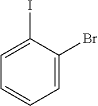 | 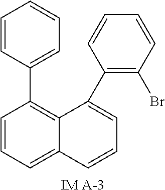 | 86 |

### Synthesis of IM B-Y

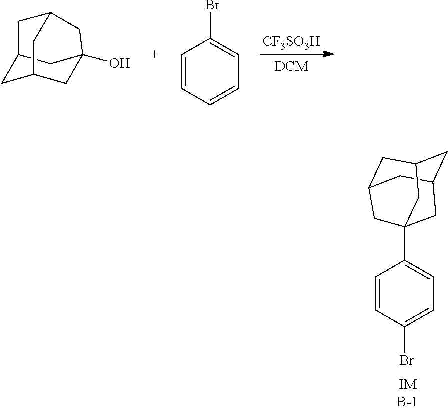

Under the protection of N₂, 1-adamantanol (100 g, 0.656 mol), bromobenzene (103.2 g, 0.656 mol), and dichloromethane (DCM) (800 mL) were added a round bottom flask, and cooled to 0-5° C., trifluoromethanesulfonic acid (CF₃SO₃H) (147.8 g, 0.985 mol) was added dropwise, after stirring at a constant temperature for 3 h, the reaction was stopped, deionized water (600 mL) was added to the reaction solution to be neutral, dichloromethane (100 mL) was added for extraction, the organic phases were mixed, anhydrous magnesium sulfate was added to remove water, filtering was performed, the obtained filtrate was concentrated, and the obtained crude product was purified by silica gel column chromatography using an eluent n-heptane to obtain TI B-1 (106.2 g, yield: 55.4%) as a white solid.

IM B-Y was synthesized with reference to the method for IM B-1, except that a raw material 2 was used instead of 1-adamantanol and a raw material 3 was used instead of bromobenzene. The used main starting materials, the synthesized intermediates and their yields are shown in Table 2.

TABLE 2

| Raw | | | Yield/ |
|---|---|---|---|
| material 2 | Raw material 3 | IM B-Y | % |
| 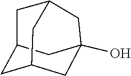 [SMILES: OC12CC3CC(CC(C3)C1)C2] | 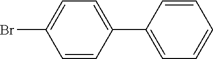 [SMILES: Brc1ccc(-c2ccccc2)cc1] | 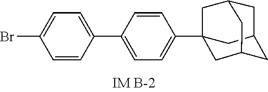 | 56.2 |
| | 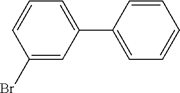 [SMILES: Brc1cccc(-c2ccccc2)c1] | 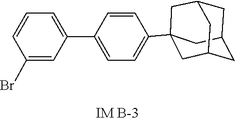 | 54.6 |
| | 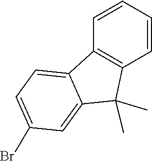 [SMILES: CC1(C)c2ccccc2-c2ccc(Br)cc21] | 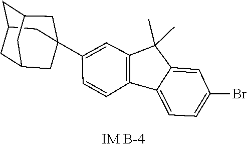 | 52.1 |
| | 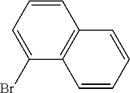 [SMILES: Brc1cccc2ccccc12] | 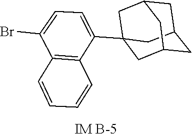 | 53.8 |
| | 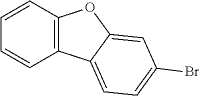 [SMILES: Brc1ccc2c(c1)oc1ccccc12] | 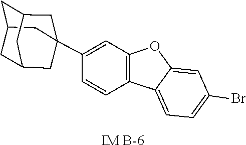 | 49.8 |
| 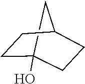 | 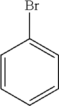 | 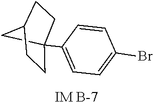 | 51.2 |
| | 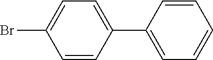 [SMILES: Brc1ccc(-c2ccccc2)cc1] | 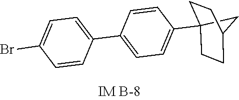 | 53.0 |
| | 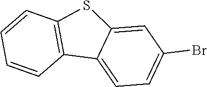 [SMILES: Brc1ccc2c(c1)sc1ccccc12] | 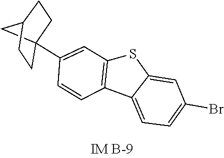 | 49.7 |
| | 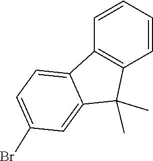 [SMILES: CC1(C)c2ccccc2-c2ccc(Br)cc21] | 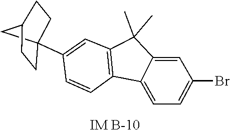 | 48.7 |
| | 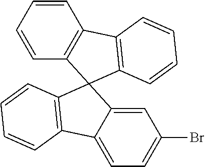 [SMILES: Brc1ccc2c(c1)C1(c3ccccc3-c3ccccc31)c1ccccc1-2] | 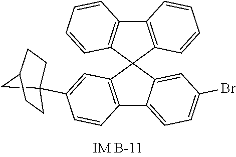 | 38. 6 |
| 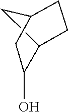 | 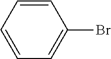 | 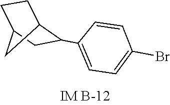 | 53.2 |
| | 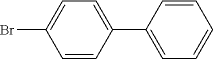 [SMILES: Brc1ccc(-c2ccccc2)cc1] | 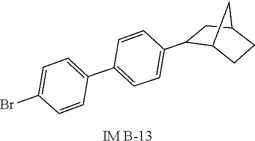 | 52.4 |
| | 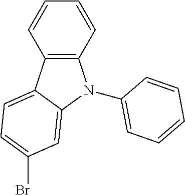 [SMILES: Brc1ccc2c3ccccc3n(-c3ccccc3)c2c1] | 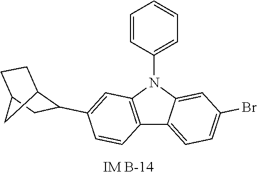 | 49.8 |
| 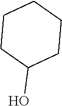 | 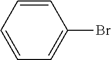 | 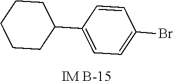 | 53.4 |
| | 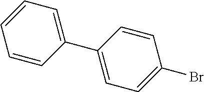 [SMILES: Brc1ccc(-c2ccccc2)cc1] | 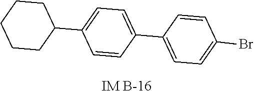 | 52.8 |
| | 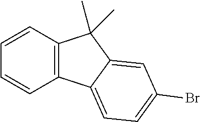 [SMILES: CC1(C)c2ccccc2-c2ccc(Br)cc21] | 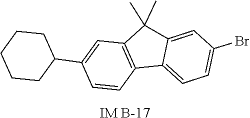 | 49.6 |
| | 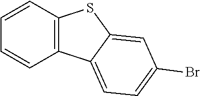 [SMILES: Brc1ccc2c(c1)sc1ccccc12] | 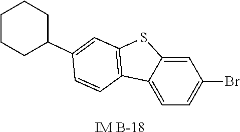 | 46.9 |
| | 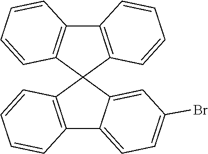 [SMILES: Brc1ccc2c(c1)C1(c3ccccc3-c3ccccc31)c1ccccc1-2] | 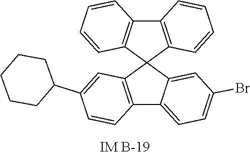 | 45.6 |

### Synthesis of IM C1-2 and IM C2-2

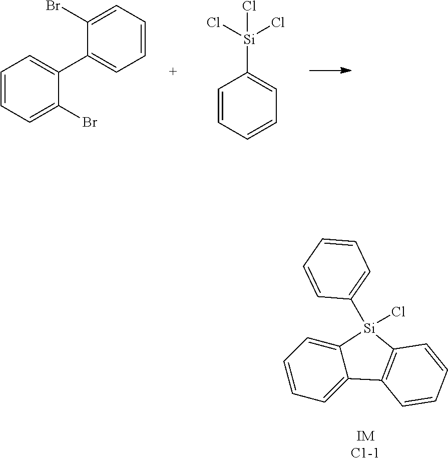 [SMILES: Brc1ccccc1-c1ccccc1Br ; Cl[Si](Cl)(Cl)c1ccccc1 ; Cl[Si]1(c2ccccc2)c2ccccc2-c2ccccc21]

Under the protection of N₂, 2,2′-dibromobiphenyl (100 g, 320 mmol) was dissolved in tetrahydrofuran (THF) (500 mL) to be clear under stirring in a 1000 mL three-necked flask, the reaction temperature was decreased to −78° C., n-butyllithium (2.5 M, 710 mmol) was added dropwise, after a reaction was carried out for 1 h, phenyltrichlorosilane (130 mL, 800 mmol) was added, the temperature was slowly raised to room temperature, a reaction was carried out under stirring for 12 h, after the reaction was stopped, the reaction solution was extracted with dichloromethane and water, an organic phase was washed with water to be neutral, dried over sodium sulfate, and allowed to pass through a column using dichloromethane as an eluent, and liquid obtained after passing through the column was concentrated, recrystallized by using dichloromethane:n-heptane (v/v)=1:4, and filtered to give IM C1-1 (46.8 g, yield: 50%).

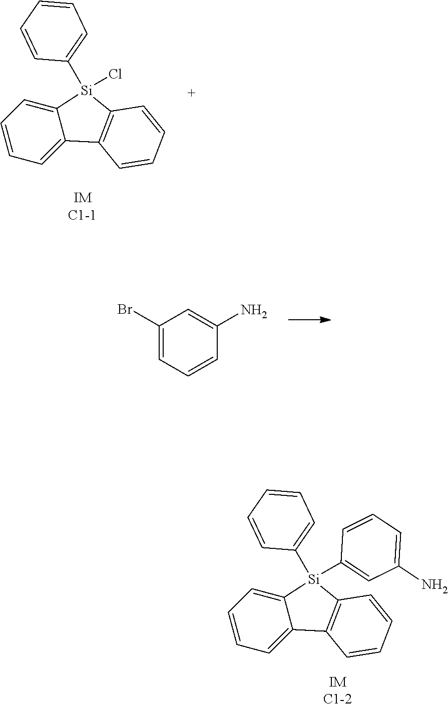 [SMILES: Cl[Si]1(c2ccccc2)c2ccccc2-c2ccccc21 ; Nc1cccc(Br)c1 ; Nc1cccc([Si]2(c3ccccc3)c3ccccc3-c3ccccc32)c1]

Under the protection of N₂, IM C1-1 (46.8 g, 160 mmol) was dissolved in THE (300 mL) to be clear under stirring in a 500 mL three-necked flask, the reaction temperature was decreased to −78° C., n-butyllithium (2.5 M, 352 mmol) was added dropwise, after a reaction was carried out for 1 h, 3-bromoaniline (55 g, 320 mmol) was added, the temperature was slowly raised to room temperature, a reaction was carried out under stirring for 12 h, after the reaction was stopped, the reaction solution was extracted with dichloromethane and water, an organic phase was washed with water to be neutral, dried over sodium sulfate, and allowed to pass through a column using dichloromethane as an eluent, and liquid obtained after passing through the column was concentrated, recrystallized by using ethyl acetate:n-heptane (v/v)=1:10, and filtered to give IM C1-2 (22.5 g, yield: 41%).

IM C2-2 was synthesized with reference to the method for IM C1-2, except that 2-bromoaniline was used instead of 3-bromoaniline, where the used main raw material, the synthesized intermediate and its yield are shown in Table 3.

TABLE 3

| Intermediates | Raw material | IM C2-2 | Yield/% |
|---|---|---|---|
| 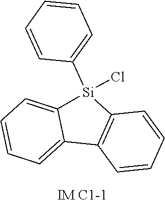 [SMILES: Cl[Si]1(c2ccccc2)c2ccccc2-c2ccccc21] | 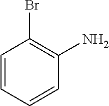 | 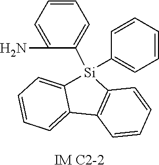 | 39 |

### Synthesis of IM C3-2

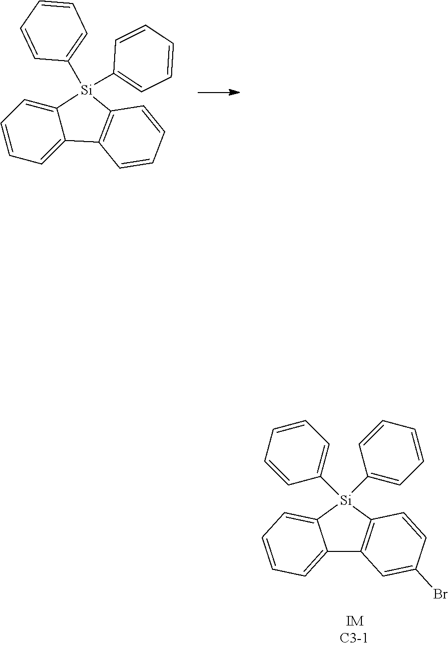 [SMILES: c1ccc([Si]2(c3ccccc3)c3ccccc3-c3ccccc32)cc1 ; Brc1ccc2c(c1)-c1ccccc1[Si]2(c1ccccc1)c1ccccc1]

Under the protection of N₂, 9,9-diphenyl-9H-9-silafluorene (27 g, 80.8 mmol) was dissolved in 300 mL of chloroform, the mixture was fully stirred at 0° C., bromine (12.9 g, 80.8 mmol) was added dropwise to the mixture, then the temperature was gradually raised to room temperature, after a reaction was carried out at room temperature for 8 h, the reaction was stopped, the reaction was quenched with water, an organic phase was washed with water for three times, dried over sodium sulfate, recrystallized with ethanol, and filtered to give IM C3-1 (23.6 g, yield: 71%).

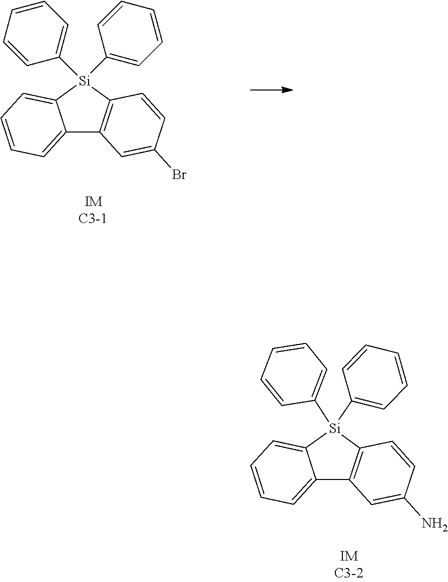 [SMILES: Brc1ccc2c(c1)-c1ccccc1[Si]2(c1ccccc1)c1ccccc1 ; Nc1ccc2c(c1)-c1ccccc1[Si]2(c1ccccc1)c1ccccc1]

Under the protection of N₂, IM C3-1 (24.8 g, 60 mmol) was dissolved in 100 mL of THF, then Cu (0.2 g) was added, a reaction was carried out under stirring at 110° C. for 12 h, after the reaction was completed, the reaction solution was extracted with dichloromethane and water, an organic phase was washed with water for three times, dried over anhydrous sodium sulfate, filtered, and allowed to pass through a silica gel column using ethyl acetate:n-heptane (v/v)=1:10 as an eluent, and liquid obtained after passing through the column was concentrated to give IM C3-2 (17 g, yield: 81%).

### Synthesis of IM C4-2

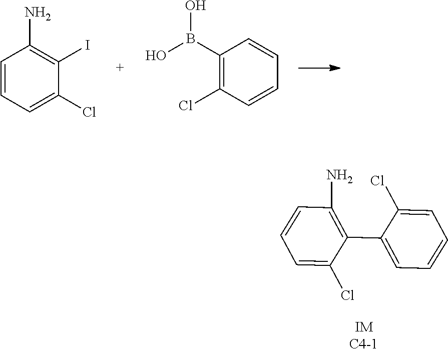 [SMILES: Nc1cccc(Cl)c1I ; OB(O)c1ccccc1Cl ; Nc1cccc(Cl)c1-c1ccccc1Cl]

Under the protection of N₂, 3-chloro-2-iodoaniline (25.3 g, 100 mmol), o-chlorophenylboric acid (15.6 g, 100 mmol), potassium carbonate (27.6 g, 200 mmol), TBAB (1.29 g, 4 mmol), Pd(PPh₃)₄ (2.31 g, 2 mmol), toluene (150 mL), ethanol (75 mL) and water (25 mL) were added to a 500 mL three-necked flask, a reaction was carried out at 80° C. for 12 h, the reaction solution was extracted with toluene and water, an organic phase was washed with water to be neutral, dried over anhydrous sodium sulfate, and allowed to pass through a silica gel column using toluene as an eluent, and liquid obtained after passing through the column was concentrated, and recrystallized by using dichloromethane:n-heptane (v/v)=1:5 to give IM C4-1 (21.9 g, yield: 91%).

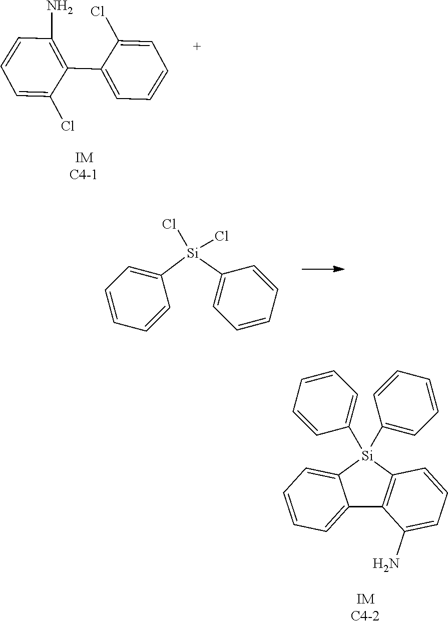 [SMILES: Nc1cccc(Cl)c1-c1ccccc1Cl ; Cl[Si](Cl)(c1ccccc1)c1ccccc1 ; Nc1cccc2c1-c1ccccc1[Si]2(c1ccccc1)c1ccccc1]

Under the protection of N₂, IM C4-1 (21.9 g, 92 mmol) was dissolved in THF (160 mL) to be clear under stirring in a 250 mL three-necked flask, the reaction temperature was decreased to −78° C., n-butyllithium (2.5 M, 202.4 mmol) was added dropwise, after a reaction was carried out for 1 h, diphenyldichlorosilane (46.6 g, 184 mmol) was added, the temperature was slowly raised to room temperature, a reaction was carried out under stirring for 12 h, after the reaction was stopped, the reaction solution was extracted with dichloromethane and water, an organic phase was washed with water to be neutral, dried over anhydrous sodium sulfate, and allowed to pass through a column using dichloromethane as an eluent, and liquid obtained after passing through the column was concentrated, recrystallized by using ethyl acetate:n-heptane (v/v)=1:20, and filtered to give IM C4-2 (12.4 g, yield: 38.5%).

### Synthesis of IM C-Z

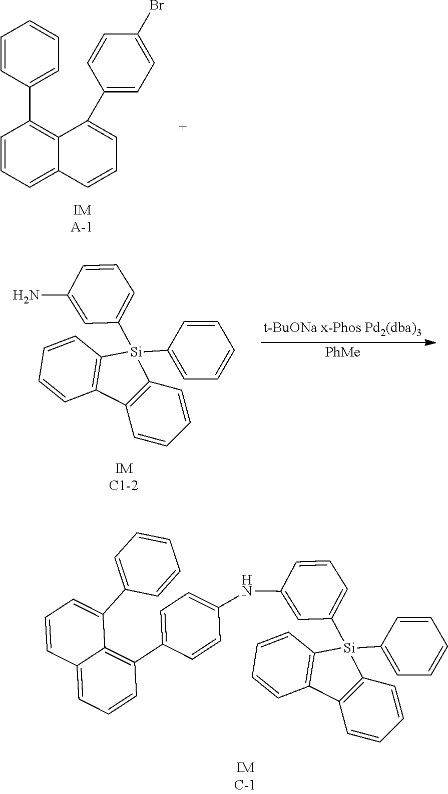 [SMILES: Brc1ccc(-c2cccc3cccc(-c4ccccc4)c23)cc1 ; Nc1cccc([Si]2(c3ccccc3)c3ccccc3-c3ccccc32)c1 ; "PhMe" ; c1ccc(-c2cccc3cccc(-c4ccc(Nc5cccc([Si]6(c7ccccc7)c7ccccc7-c7ccccc76)c5)cc4)c23)cc1]

Under the protection of N₂, IM A-1 (0.056 mol, 20 g), IM C1-2 (0.056 mol, 19.57 g), and toluene (160 mL) were added into a 250 mL three-necked round bottom flask, stirred under reflux at 108° C. for 30 min, and cooled to 70-80° C., sodium tert-butoxide (t-BuONa) (0.112 mol, 10.76 g), 2-dicyclohexylphosphino-2′,4′,6′-triisopropylbiphenyl (x-Phos) (0.001 mol, 0.53 g) and tris(dibenzylideneacetone)dipalladium (Pd₂(dba)₃) (0.0005 mol, 0.4576 g) were added, after the temperature of the system was stable, and a reaction was carried out under reflux for 4 h, the reaction was stopped, after the reaction solution was cooled to room temperature, 100 mL of deionized water was added, the reaction solution was extracted with toluene/water, an organic phase was washed with water to be neutral, anhydrous magnesium sulfate was added to remove water, filtering was performed, and the obtained filtrate was concentrated, and allowed to pass through a silica gel column using an eluent dichloromethane:n-heptane (v/v)=1:4 as an eluent to give IM C-1 (29.9 g, yield: 85%) as a white solid.

IM C-Z listed in Table 4 was synthesized with reference to the method for IM C-1, except that a raw material 4 was used instead of IMA-1 and a raw material 5 was used instead of IM C1-2. The used main raw materials, the synthesized intermediates and their yields are shown in Table 4.

TABLE 4

| | | | Yield/ |
|---|---|---|---|
| Raw material 4 | Raw material 5 | IM C-Z | % |
| 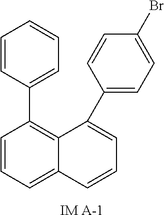 | 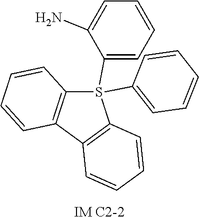 | 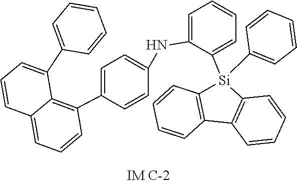 | 81 |
| | 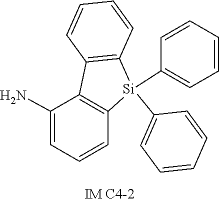 [SMILES: Nc1cccc2c1-c1ccccc1[Si]2(c1ccccc1)c1ccccc1] | 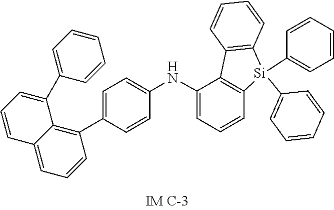 | 84 |
| | 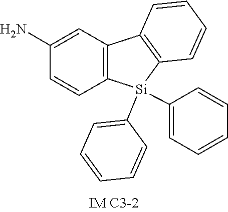 [SMILES: Nc1ccc2c(c1)-c1ccccc1[Si]2(c1ccccc1)c1ccccc1] | 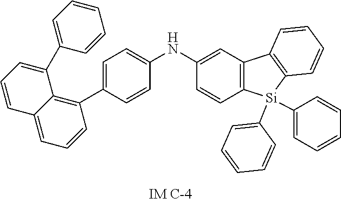 | 83 |
| | 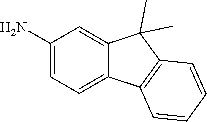 [SMILES: CC1(C)c2ccccc2-c2ccc(N)cc21] | 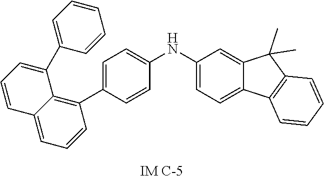 | 82 |
| | 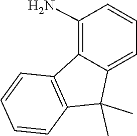 [SMILES: CC1(C)c2ccccc2-c2c(N)cccc21] | 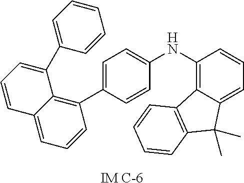 | 75 |
| | 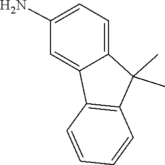 [SMILES: CC1(C)c2ccccc2-c2cc(N)ccc21] | 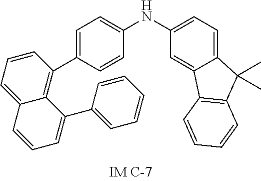 | 70 |
| | 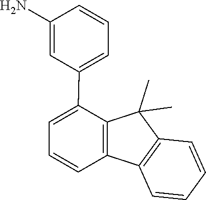 [SMILES: CC1(C)c2ccccc2-c2cccc(-c3cccc(N)c3)c21] | 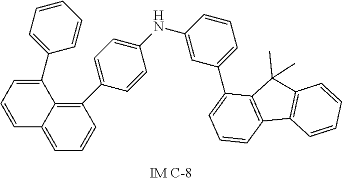 | 81 |
| | 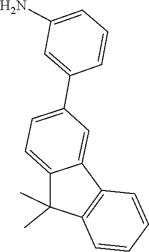 [SMILES: CC1(C)c2ccccc2-c2cc(-c3cccc(N)c3)ccc21] | 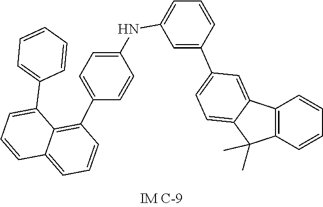 | 84 |
| | 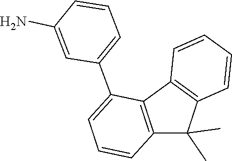 [SMILES: CC1(C)c2ccccc2-c2c(-c3cccc(N)c3)cccc21] | 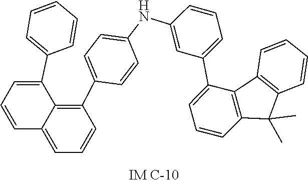 | 81 |
| | 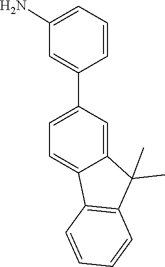 [SMILES: CC1(C)c2ccccc2-c2ccc(-c3cccc(N)c3)cc21] | 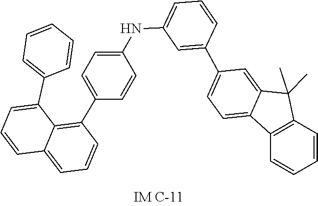 | 70 |
| | 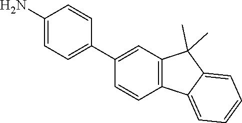 [SMILES: CC1(C)c2ccccc2-c2ccc(-c3ccc(N)cc3)cc21] | 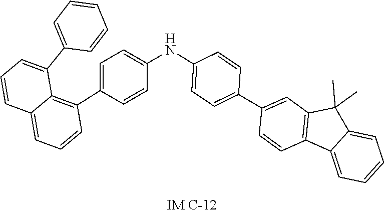 | 79 |
| | 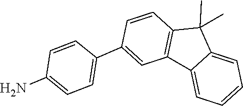 [SMILES: CC1(C)c2ccccc2-c2cc(-c3ccc(N)cc3)ccc21] | 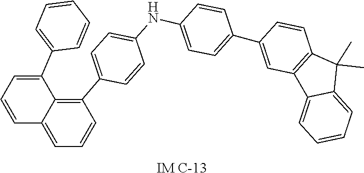 | 72 |
| | 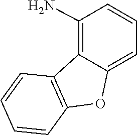 [SMILES: Nc1cccc2oc3ccccc3c12] | 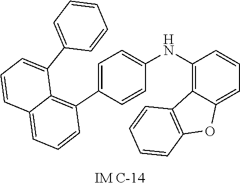 | 73 |
| | 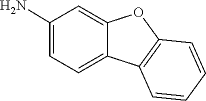 [SMILES: Nc1ccc2c(c1)oc1ccccc12] | 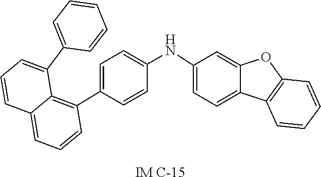 | 82 |
| | 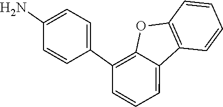 [SMILES: Nc1ccc(-c2cccc3c2oc2ccccc23)cc1] | 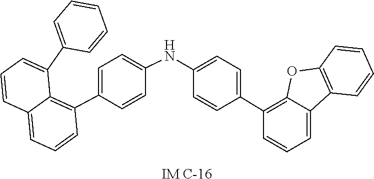 | 79 |
| | 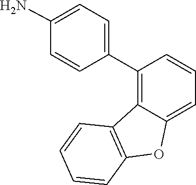 [SMILES: Nc1ccc(-c2cccc3oc4ccccc4c23)cc1] | 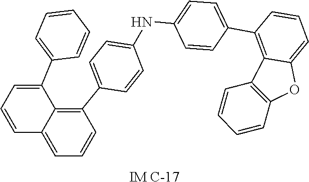 | 81 |
| | 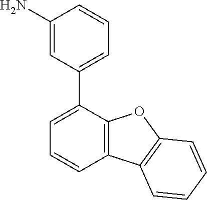 [SMILES: Nc1cccc(-c2cccc3c2oc2ccccc23)c1] | 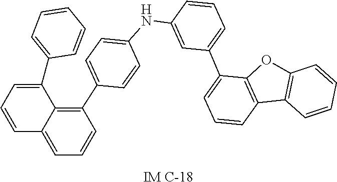 | 76 |
| | 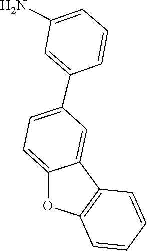 [SMILES: Nc1cccc(-c2ccc3oc4ccccc4c3c2)c1] | 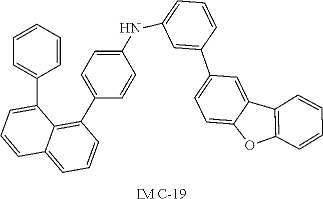 | 65 |
| | 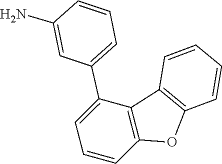 [SMILES: Nc1cccc(-c2cccc3oc4ccccc4c23)c1] | 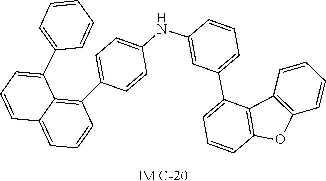 | 67 |
| | 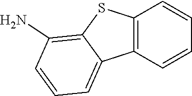 [SMILES: Nc1cccc2c1sc1ccccc12] | 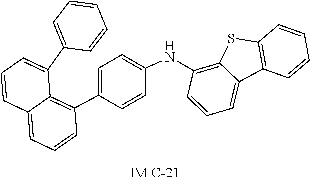 | 72 |
| | 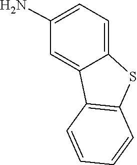 [SMILES: Nc1ccc2sc3ccccc3c2c1] | 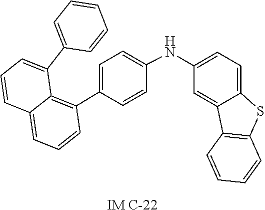 | 79 |
| | 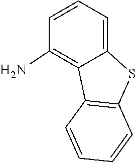 [SMILES: Nc1cccc2sc3ccccc3c12] | 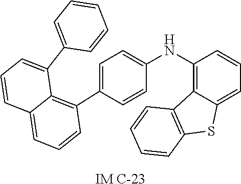 | 64 |
| | 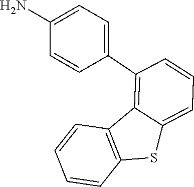 [SMILES: Nc1ccc(-c2cccc3sc4ccccc4c23)cc1] | 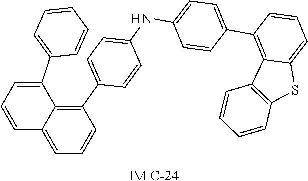 | 82 |
| | 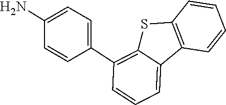 [SMILES: Nc1ccc(-c2cccc3c2sc2ccccc23)cc1] | 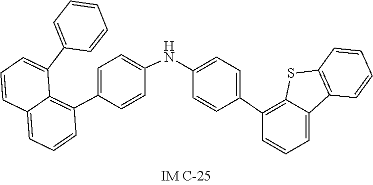 | 76 |
| | 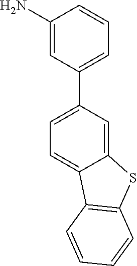 [SMILES: Nc1cccc(-c2ccc3c(c2)sc2ccccc23)c1] | 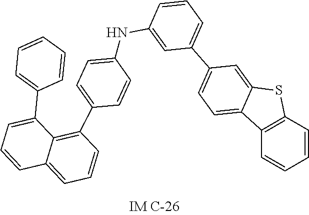 | 73 |
| | 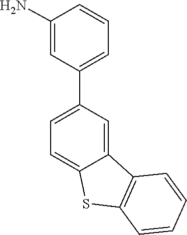 [SMILES: Nc1cccc(-c2ccc3sc4ccccc4c3c2)c1] | 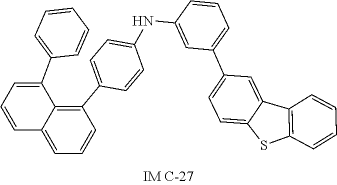 | 76 |
| | 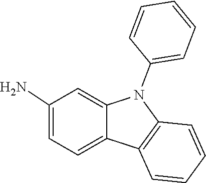 [SMILES: Nc1ccc2c3ccccc3n(-c3ccccc3)c2c1] | 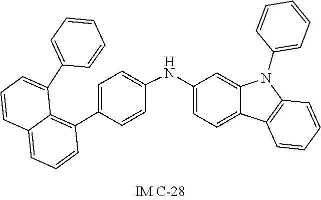 | 79 |
| | 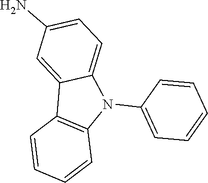 [SMILES: Nc1ccc2c(c1)c1ccccc1n2-c1ccccc1] | 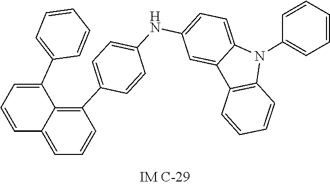 | 76 |
| | 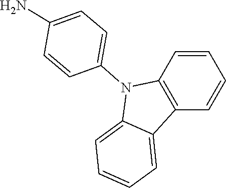 [SMILES: Nc1ccc(-n2c3ccccc3c3ccccc32)cc1] | 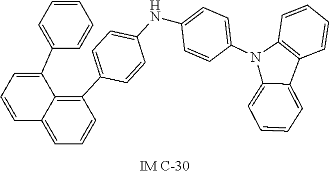 | 81 |
| | 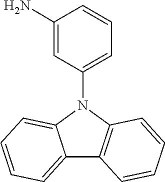 [SMILES: Nc1cccc(-n2c3ccccc3c3ccccc32)c1] | 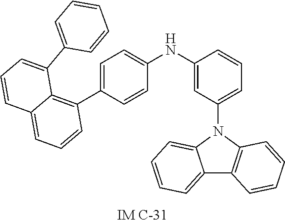 | 78 |
| | 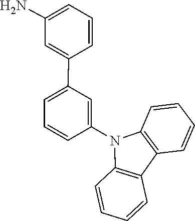 [SMILES: Nc1cccc(-c2cccc(-n3c4ccccc4c4ccccc43)c2)c1] | 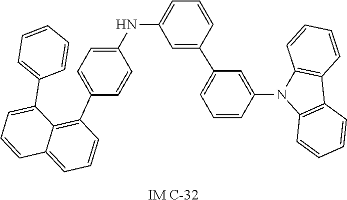 | 60 |
| | 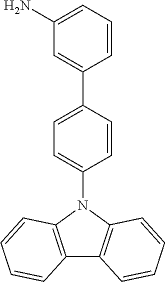 [SMILES: Nc1cccc(-c2ccc(-n3c4ccccc4c4ccccc43)cc2)c1] | 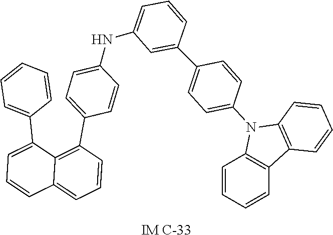 | 69 |
| | 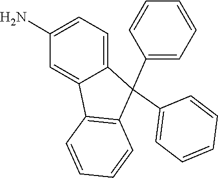 [SMILES: Nc1ccc2c(c1)-c1ccccc1C2(c1ccccc1)c1ccccc1] | 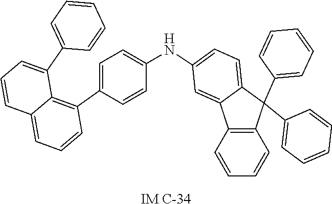 | 80 |
| | 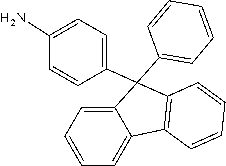 [SMILES: Nc1ccc(C2(c3ccccc3)c3ccccc3-c3ccccc32)cc1] | 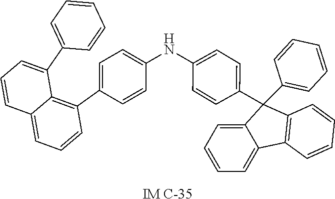 | 75 |
| | 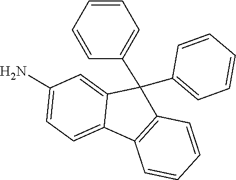 [SMILES: Nc1ccc2c(c1)C(c1ccccc1)(c1ccccc1)c1ccccc1-2] | 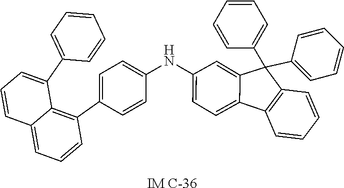 | 76 |
| | 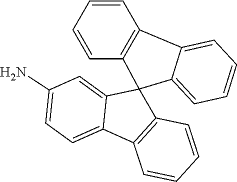 [SMILES: Nc1ccc2c(c1)C1(c3ccccc3-c3ccccc31)c1ccccc1-2] | 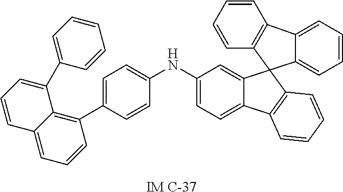 | 82 |
| 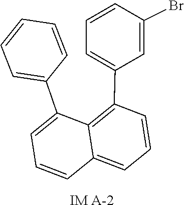 | 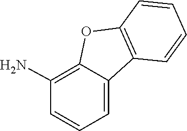 [SMILES: Nc1cccc2c1oc1ccccc12] | 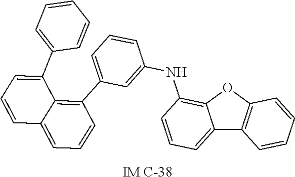 | 79 |
| | 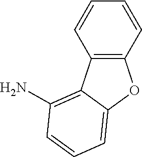 [SMILES: Nc1cccc2oc3ccccc3c12] | 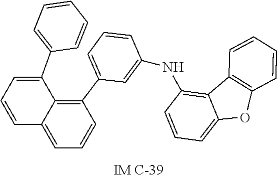 | 65 |
| | 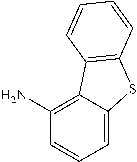 [SMILES: Nc1cccc2sc3ccccc3c12] | 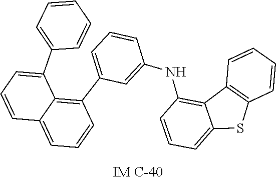 | 46 |
| | 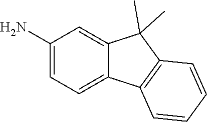 [SMILES: CC1(C)c2ccccc2-c2ccc(N)cc21] | 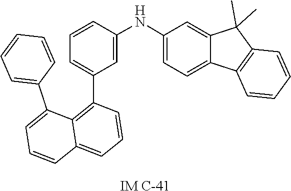 | 77 |
| | 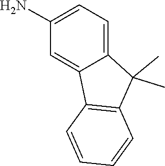 [SMILES: CC1(C)c2ccccc2-c2cc(N)ccc21] | 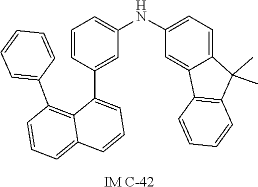 | 79 |
| | 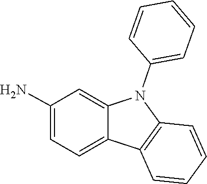 [SMILES: Nc1ccc2c3ccccc3n(-c3ccccc3)c2c1] | 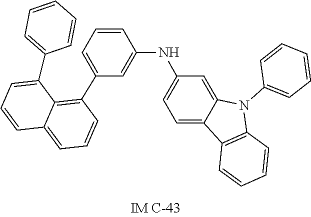 | 76 |
| 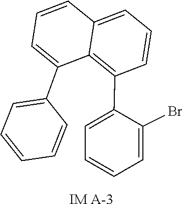 | 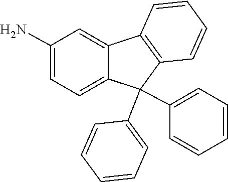 [SMILES: Nc1ccc2c(c1)-c1ccccc1C2(c1ccccc1)c1ccccc1] | 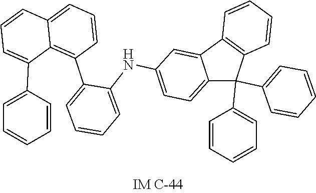 | 69 |
| | 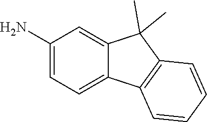 [SMILES: CC1(C)c2ccccc2-c2ccc(N)cc21] | 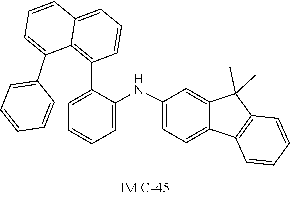 | 71 |
| | 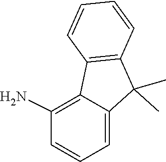 [SMILES: CC1(C)c2ccccc2-c2c(N)cccc21] | 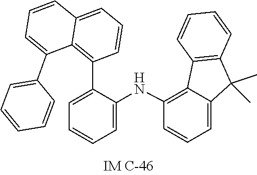 | 54 |
| | 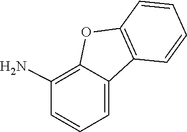 [SMILES: Nc1cccc2c1oc1ccccc12] | 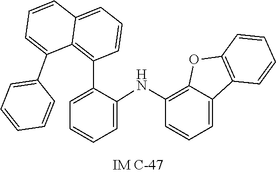 | 56 |
| | 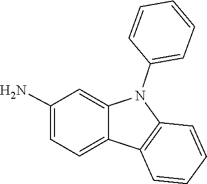 [SMILES: Nc1ccc2c3ccccc3n(-c3ccccc3)c2c1] | 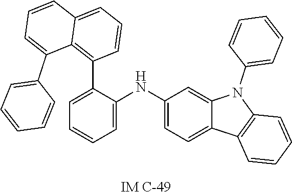 | 61 |

### Synthesis of Compound X

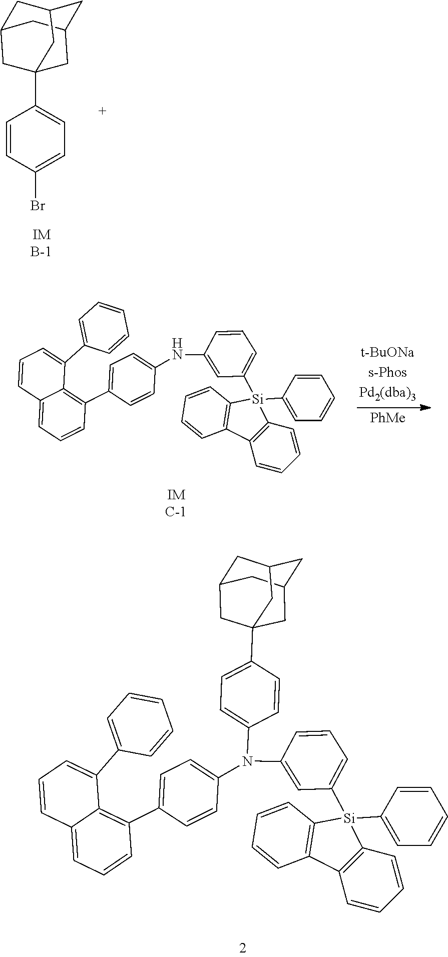 [SMILES: Brc1ccc(C23CC4CC(CC(C4)C2)C3)cc1 ; c1ccc(-c2cccc3cccc(-c4ccc(Nc5cccc([Si]6(c7ccccc7)c7ccccc7-c7ccccc76)c5)cc4)c23)cc1 ; "PhMe" ; c1ccc(-c2cccc3cccc(-c4ccc(N(c5ccc(C67CC8CC(CC(C8)C6)C7)cc5)c5cccc([Si]6(c7ccccc7)c7ccccc7-c7ccccc76)c5)cc4)c23)cc1]

Under the protection of N₂, IM B-1 (8.85 g, 30.4 mmol), IM C-1 (19.08 g, 30.4 mmol), and toluene (100 mL) were added into a 250 mL three-necked round bottom flask, and stirred under reflux at 108° C. to be dissolved so as to obtain a clear solution, the solution was cooled to 70-80° C., sodium tert-butoxide (4.4 g, 45.7 mmol), 2-dicyclohexylphosphino-2′,6′-dimethoxybiphenyl (s-Phos) (0.25 g, 0.61 mmol), and Pd₂(dba)₃ (0.28 g, 0.330 mmol) were added, after a reaction was carried out under reflux for 6 h, the reaction was stopped, the reaction temperature was reduced to room temperature, the reaction solution was extracted with toluene and deionized water, washing was performed with water to be neutral, anhydrous magnesium sulfate was added to remove water, a product with water removed was allowed to pass through a column using ethyl acetate:n-heptane (v/v)=1:10 as an eluent, liquid obtained after pass through the column was concentrated, recrystallized by using toluene and n-heptane, and filtered to give a compound 2 (21.7 g, yield: 85%) as a white solid; mass spectrum (m/z)=838.38 [M+H]⁺.

Compounds shown in Table 5 were synthesized with reference to the method for the compound 2, except that a raw material 6 was used instead of IM B-1 and a raw material 7 was used instead of IM C-1. The used main starting materials, the synthesized compounds and their yields, and the mass spectra are shown in Table 5.

TABLE 5

| | | | | Mass |
|---|---|---|---|---|
| | | | | spectrum |
| | | | Yield/ | (m/z)/ |
| Raw material 6 | Raw material 7 | Compound | % | [M + H]⁺ |
| 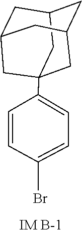 [SMILES: Brc1ccc(C23CC4CC(CC(C4)C2)C3)cc1] | 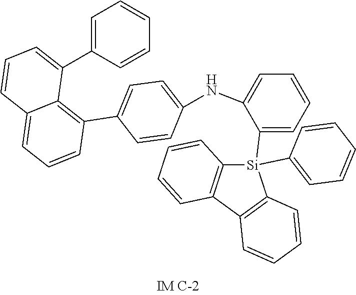 [SMILES: c1ccc(-c2cccc3cccc(-c4ccc(Nc5ccccc5[Si]5(c6ccccc6)c6ccccc6-c6ccccc65)cc4)c23)cc1] | 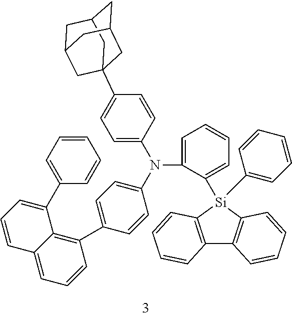 [SMILES: c1ccc(-c2cccc3cccc(-c4ccc(N(c5ccc(C67CC8CC(CC(C8)C6)C7)cc5)c5ccccc5[Si]5(c6ccccc6)c6ccccc6-c6ccccc65)cc4)c23)cc1] | 82 | 838.38 |
| | 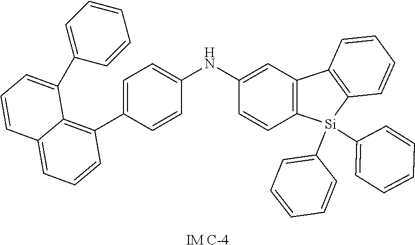 | 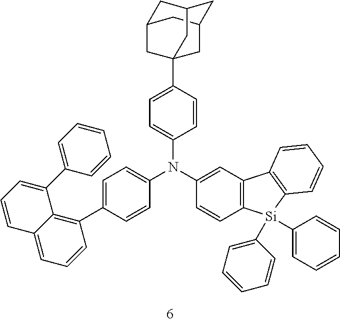 [SMILES: c1ccc(-c2cccc3cccc(-c4ccc(N(c5ccc(C67CC8CC(CC(C8)C6)C7)cc5)c5ccc6c(c5)-c5ccccc5[Si]6(c5ccccc5)c5ccccc5)cc4)c23)cc1] | 83 | 838.38 |
| | 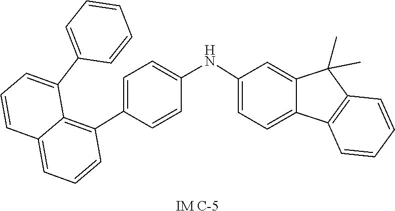 | 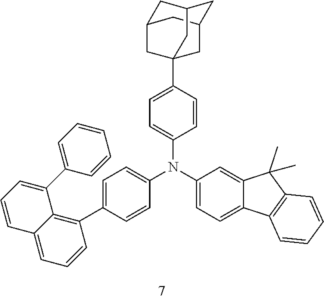 [SMILES: CC1(C)c2ccccc2-c2ccc(N(c3ccc(-c4cccc5cccc(-c6ccccc6)c45)cc3)c3ccc(C45CC6CC(CC(C6)C4)C5)cc3)cc21] | 85 | 678.37 |
| | 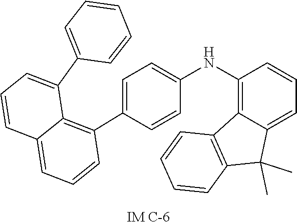 | 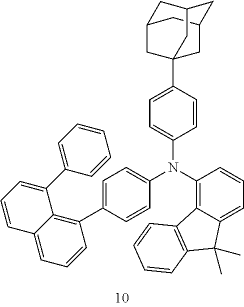 [SMILES: CC1(C)c2ccccc2-c2c(N(c3ccc(-c4cccc5cccc(-c6ccccc6)c45)cc3)c3ccc(C45CC6CC(CC(C6)C4)C5)cc3)cccc21] | 81 | 698.37 |
| | 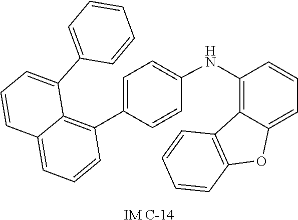 | 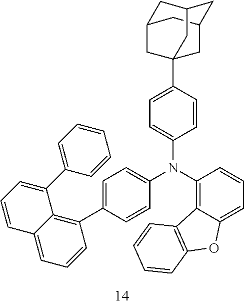 [SMILES: c1ccc(-c2cccc3cccc(-c4ccc(N(c5ccc(C67CC8CC(CC(C8)C6)C7)cc5)c5cccc6oc7ccccc7c56)cc4)c23)cc1] | 82 | 672.32 |
| | 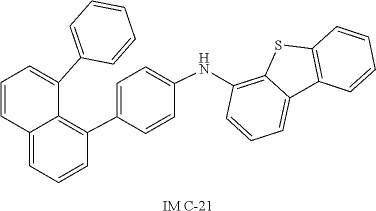 | 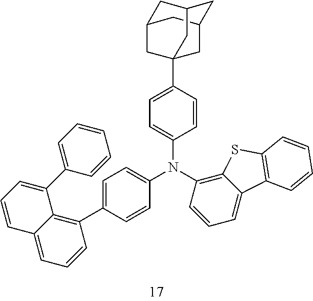 [SMILES: c1ccc(-c2cccc3cccc(-c4ccc(N(c5ccc(C67CC8CC(CC(C8)C6)C7)cc5)c5cccc6c5sc5ccccc56)cc4)c23)cc1] | 86 | 688.30 |
| | 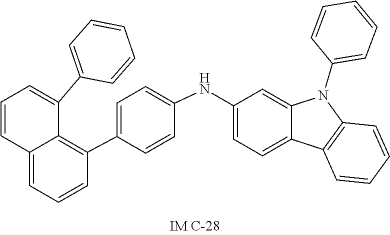 [SMILES: c1ccc(-c2cccc3cccc(-c4ccc(Nc5ccc6c7ccccc7n(-c7ccccc7)c6c5)cc4)c23)cc1] | 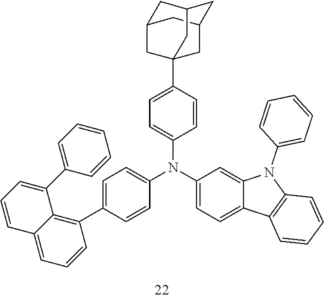 [SMILES: c1ccc(-c2cccc3cccc(-c4ccc(N(c5ccc(C67CC8CC(CC(C8)C6)C7)cc5)c5ccc6c7ccccc7n(-c7ccccc7)c6c5)cc4)c23)cc1] | 79 | 747.37 |
| | 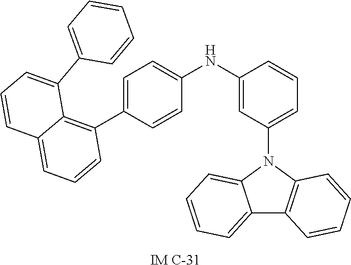 | 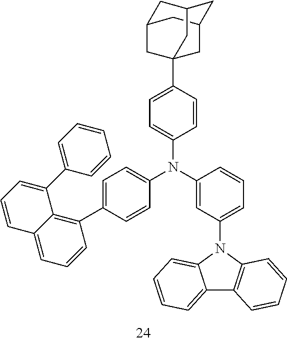 [SMILES: c1ccc(-c2cccc3cccc(-c4ccc(N(c5ccc(C67CC8CC(CC(C8)C6)C7)cc5)c5cccc(-n6c7ccccc7c7ccccc76)c5)cc4)c23)cc1] | 78 | 747.37 |
| | 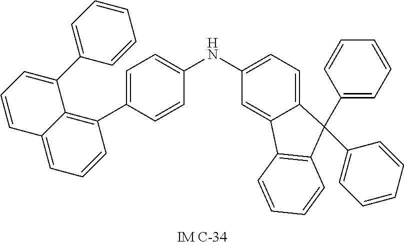 | 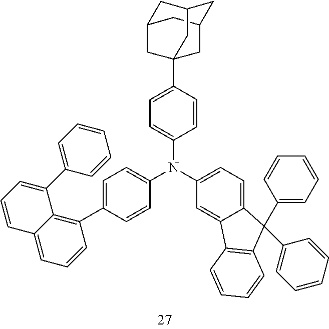 [SMILES: c1ccc(-c2cccc3cccc(-c4ccc(N(c5ccc(C67CC8CC(CC(C8)C6)C7)cc5)c5ccc6c(c5)-c5ccccc5C6(c5ccccc5)c5ccccc5)cc4)c23)cc1] | 76 | 822.40 |
| | 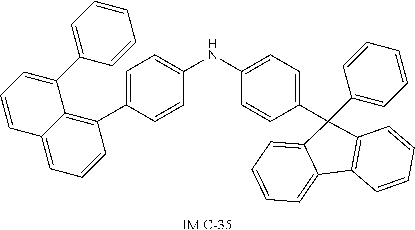 | 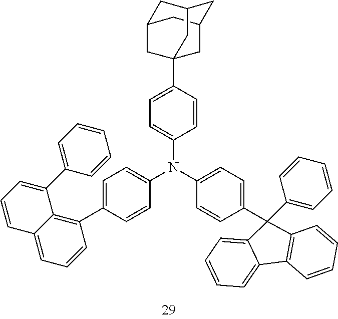 [SMILES: c1ccc(-c2cccc3cccc(-c4ccc(N(c5ccc(C67CC8CC(CC(C8)C6)C7)cc5)c5ccc(C6(c7ccccc7)c7ccccc7-c7ccccc76)cc5)cc4)c23)cc1] | 81 | 822.40 |
| | 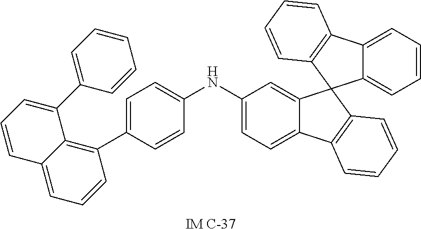 | 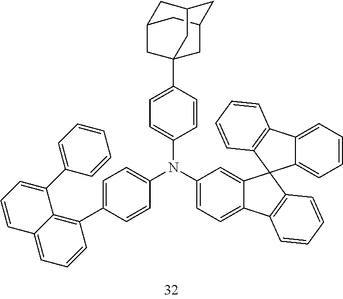 [SMILES: c1ccc(-c2cccc3cccc(-c4ccc(N(c5ccc(C67CC8CC(CC(C8)C6)C7)cc5)c5ccc6c(c5)C5(c7ccccc7-c7ccccc75)c5ccccc5-6)cc4)c23)cc1] | 82 | 820.39 |
| | 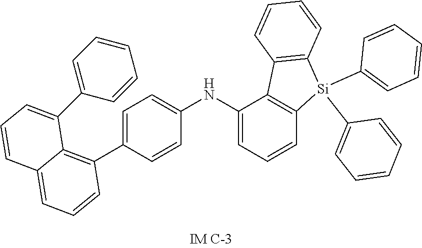 [SMILES: c1ccc(-c2cccc3cccc(-c4ccc(Nc5cccc6c5-c5ccccc5[Si]6(c5ccccc5)c5ccccc5)cc4)c23)cc1] | 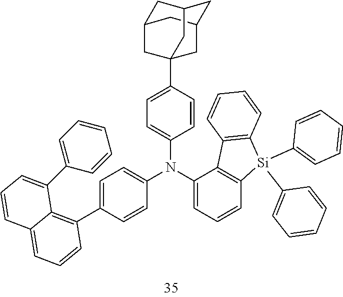 [SMILES: c1ccc(-c2cccc3cccc(-c4ccc(N(c5ccc(C67CC8CC(CC(C8)C6)C7)cc5)c5cccc6c5-c5ccccc5[Si]6(c5ccccc5)c5ccccc5)cc4)c23)cc1] | 83 | 838.38 |
| | 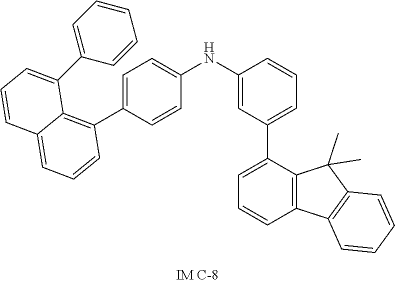 [SMILES: CC1(C)c2ccccc2-c2cccc(-c3cccc(Nc4ccc(-c5cccc6cccc(-c7ccccc7)c56)cc4)c3)c21] | 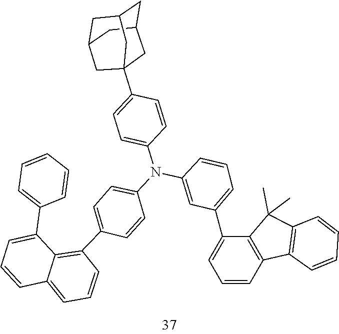 [SMILES: CC1(C)c2ccccc2-c2cccc(-c3cccc(N(c4ccc(-c5cccc6cccc(-c7ccccc7)c56)cc4)c4ccc(C56CC7CC(CC(C7)C5)C6)cc4)c3)c21] | 79 | 774.40 |
| | 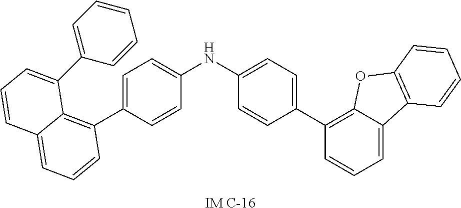 | 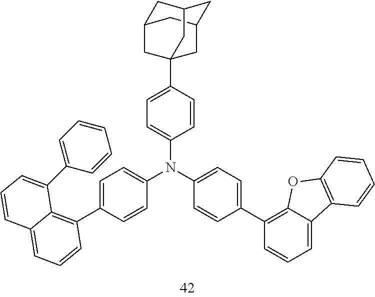 [SMILES: c1ccc(-c2cccc3cccc(-c4ccc(N(c5ccc(-c6cccc7c6oc6ccccc67)cc5)c5ccc(C67CC8CC(CC(C8)C6)C7)cc5)cc4)c23)cc1] | 76 | 748.35 |
| | 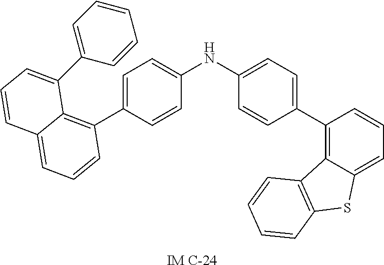 | 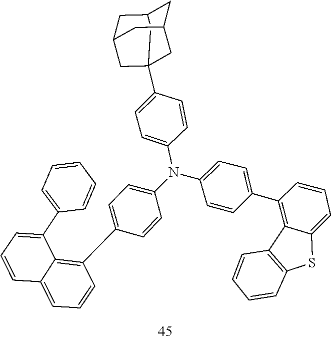 [SMILES: c1ccc(-c2cccc3cccc(-c4ccc(N(c5ccc(-c6cccc7sc8ccccc8c67)cc5)c5ccc(C67CC8CC(CC(C8)C6)C7)cc5)cc4)c23)cc1] | 75 | 764.33 |
| | 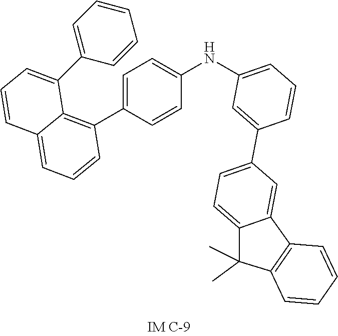 [SMILES: CC1(C)c2ccccc2-c2cc(-c3cccc(Nc4ccc(-c5cccc6cccc(-c7ccccc7)c56)cc4)c3)ccc21] | 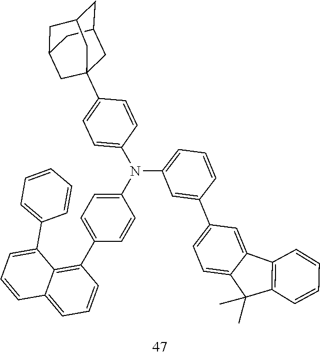 [SMILES: CC1(C)c2ccccc2-c2cc(-c3cccc(N(c4ccc(-c5cccc6cccc(-c7ccccc7)c56)cc4)c4ccc(C56CC7CC(CC(C7)C5)C6)cc4)c3)ccc21] | 78 | 774.40 |
| | 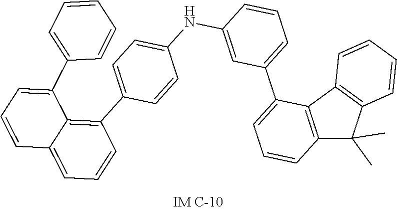 [SMILES: CC1(C)c2ccccc2-c2c(-c3cccc(Nc4ccc(-c5cccc6cccc(-c7ccccc7)c56)cc4)c3)cccc21] | 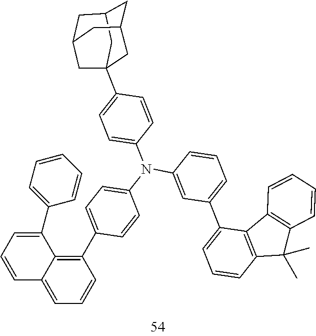 [SMILES: CC1(C)c2ccccc2-c2c(-c3cccc(N(c4ccc(-c5cccc6cccc(-c7ccccc7)c56)cc4)c4ccc(C56CC7CC(CC(C7)C5)C6)cc4)c3)cccc21] | 76 | 774.40 |
| | 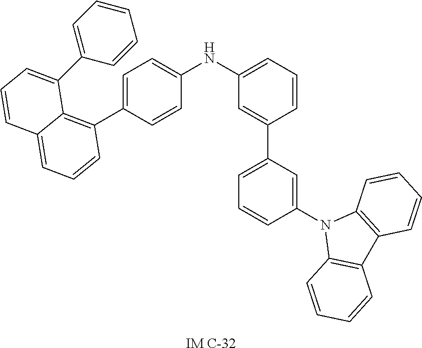 | 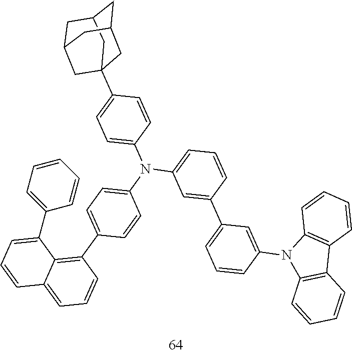 [SMILES: c1ccc(-c2cccc3cccc(-c4ccc(N(c5ccc(C67CC8CC(CC(C8)C6)C7)cc5)c5cccc(-c6cccc(-n7c8ccccc8c8ccccc87)c6)c5)cc4)c23)cc1] | 74 | 823.40 |
| | 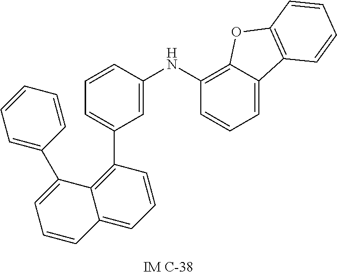 | 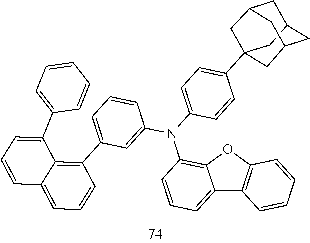 [SMILES: c1ccc(-c2cccc3cccc(-c4cccc(N(c5ccc(C67CC8CC(CC(C8)C6)C7)cc5)c5cccc6c5oc5ccccc56)c4)c23)cc1] | 65 | 672.32 |
| | 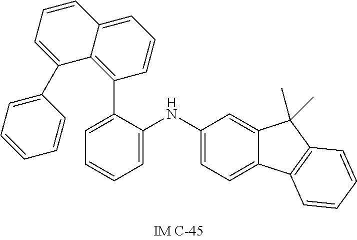 [SMILES: CC1(C)c2ccccc2-c2ccc(Nc3ccccc3-c3cccc4cccc(-c5ccccc5)c34)cc21] | 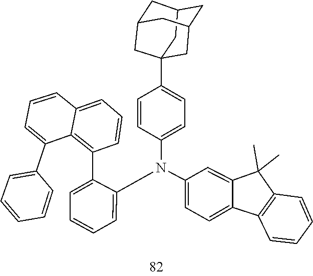 [SMILES: CC1(C)c2ccccc2-c2ccc(N(c3ccc(C45CC6CC(CC(C6)C4)C5)cc3)c3ccccc3-c3cccc4cccc(-c5ccccc5)c34)cc21] | 64 | 697.37 |
| 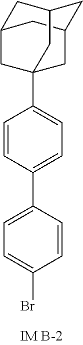 [SMILES: Brc1ccc(-c2ccc(C34CC5CC(CC(C5)C3)C4)cc2)cc1] | 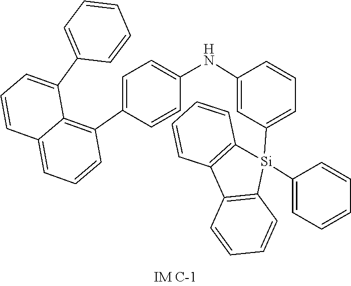 [SMILES: c1ccc(-c2cccc3cccc(-c4ccc(Nc5cccc([Si]6(c7ccccc7)c7ccccc7-c7ccccc76)c5)cc4)c23)cc1] | 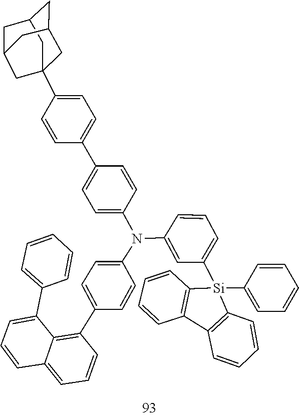 [SMILES: c1ccc(-c2cccc3cccc(-c4ccc(N(c5ccc(-c6ccc(C78CC9CC(CC(C9)C7)C8)cc6)cc5)c5cccc([Si]6(c7ccccc7)c7ccccc7-c7ccccc76)c5)cc4)c23)cc1] | 82 | 914.41 |
| | 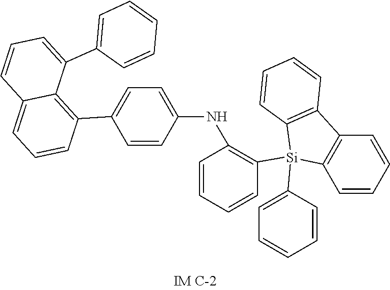 [SMILES: c1ccc(-c2cccc3cccc(-c4ccc(Nc5ccccc5[Si]5(c6ccccc6)c6ccccc6-c6ccccc65)cc4)c23)cc1] | 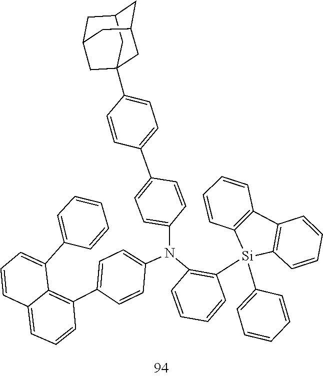 [SMILES: c1ccc(-c2cccc3cccc(-c4ccc(N(c5ccc(-c6ccc(C78CC9CC(CC(C9)C7)C8)cc6)cc5)c5ccccc5[Si]5(c6ccccc6)c6ccccc6-c6ccccc65)cc4)c23)cc1] | 79 | 914.41 |
| | 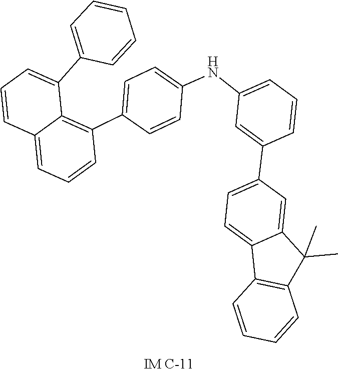 [SMILES: CC1(C)c2ccccc2-c2ccc(-c3cccc(Nc4ccc(-c5cccc6cccc(-c7ccccc7)c56)cc4)c3)cc21] | 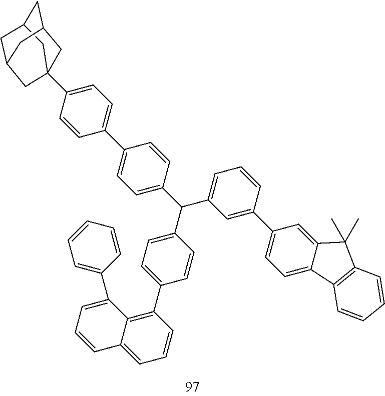 [SMILES: CC1(C)c2ccccc2-c2ccc(-c3cccc(C(c4ccc(-c5ccc(C67CC8CC(CC(C8)C6)C7)cc5)cc4)c4ccc(-c5cccc6cccc(-c7ccccc7)c56)cc4)c3)cc21] | 76 | 850.43 |
| | 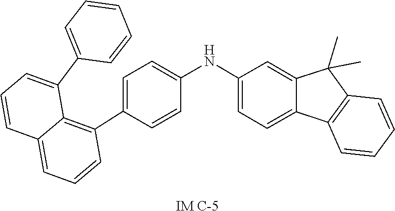 | 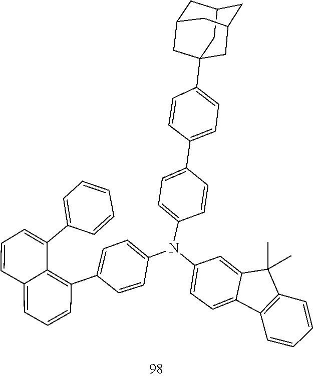 [SMILES: CC1(C)c2ccccc2-c2ccc(N(c3ccc(-c4ccc(C56CC7CC(CC(C7)C5)C6)cc4)cc3)c3ccc(-c4cccc5cccc(-c6ccccc6)c45)cc3)cc21] | 81 | 774.40 |
| | 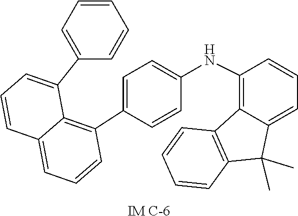 | 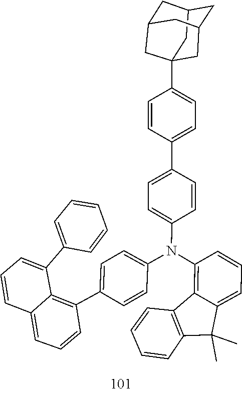 [SMILES: CC1(C)c2ccccc2-c2c(N(c3ccc(-c4ccc(C56CC7CC(CC(C7)C5)C6)cc4)cc3)c3ccc(-c4cccc5cccc(-c6ccccc6)c45)cc3)cccc21] | 68 | 774.30 |
| | 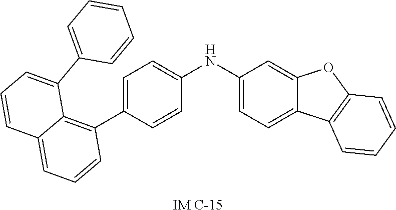 | 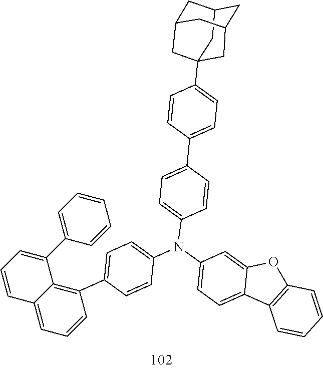 [SMILES: c1ccc(-c2cccc3cccc(-c4ccc(N(c5ccc(-c6ccc(C78CC9CC(CC(C9)C7)C8)cc6)cc5)c5ccc6c(c5)oc5ccccc56)cc4)c23)cc1] | 79 | 748.35 |
| | 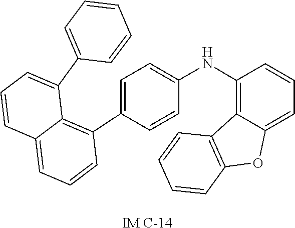 | 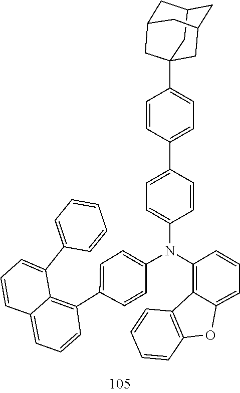 [SMILES: c1ccc(-c2cccc3cccc(-c4ccc(N(c5ccc(-c6ccc(C78CC9CC(CC(C9)C7)C8)cc6)cc5)c5cccc6oc7ccccc7c56)cc4)c23)cc1] | 64 | 748.35 |
| | 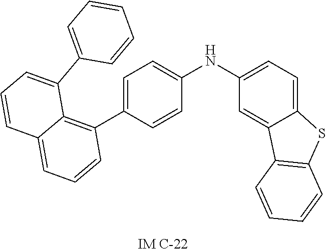 | 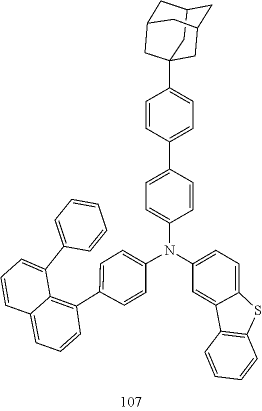 [SMILES: c1ccc(-c2cccc3cccc(-c4ccc(N(c5ccc(-c6ccc(C78CC9CC(CC(C9)C7)C8)cc6)cc5)c5ccc6sc7ccccc7c6c5)cc4)c23)cc1] | 72 | 764.33 |
| | 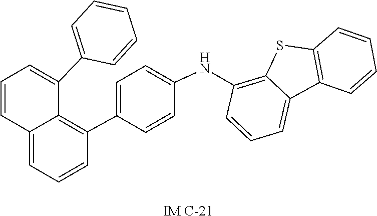 | 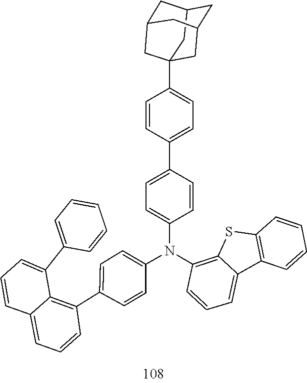 [SMILES: c1ccc(-c2cccc3cccc(-c4ccc(N(c5ccc(-c6ccc(C78CC9CC(CC(C9)C7)C8)cc6)cc5)c5cccc6c5sc5ccccc56)cc4)c23)cc1] | 68 | 764.33 |
| | 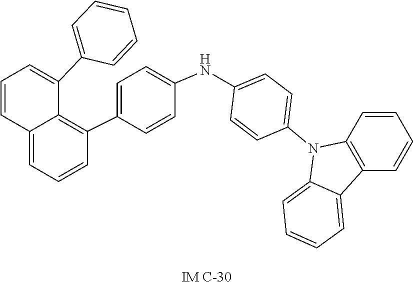 | 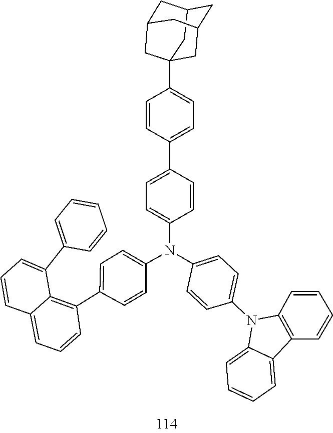 [SMILES: c1ccc(-c2cccc3cccc(-c4ccc(N(c5ccc(-c6ccc(C78CC9CC(CC(C9)C7)C8)cc6)cc5)c5ccc(-n6c7ccccc7c7ccccc76)cc5)cc4)c23)cc1] | 79 | 823.40 |
| | 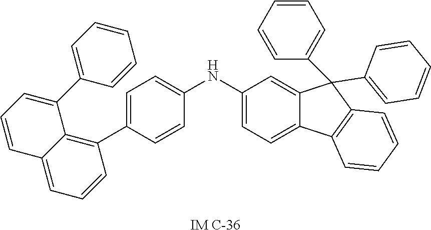 | 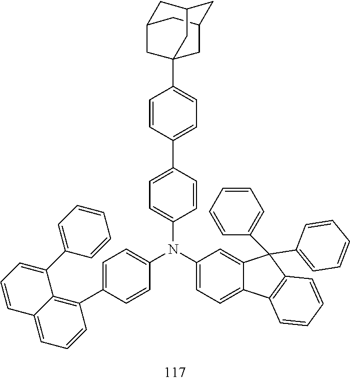 [SMILES: c1ccc(-c2cccc3cccc(-c4ccc(N(c5ccc(-c6ccc(C78CC9CC(CC(C9)C7)C8)cc6)cc5)c5ccc6c(c5)C(c5ccccc5)(c5ccccc5)c5ccccc5-6)cc4)c23)cc1] | 76 | 898.43 |
| | 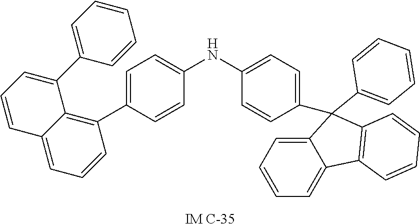 | 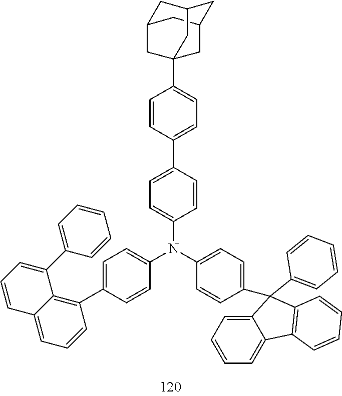 [SMILES: c1ccc(-c2cccc3cccc(-c4ccc(N(c5ccc(-c6ccc(C78CC9CC(CC(C9)C7)C8)cc6)cc5)c5ccc(C6(c7ccccc7)c7ccccc7-c7ccccc76)cc5)cc4)c23)cc1] | 75 | 898.43 |
| | 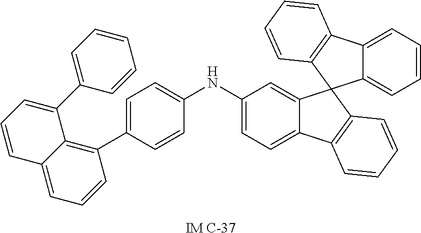 | 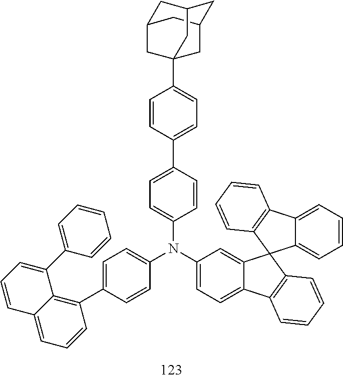 [SMILES: c1ccc(-c2cccc3cccc(-c4ccc(N(c5ccc(-c6ccc(C78CC9CC(CC(C9)C7)C8)cc6)cc5)c5ccc6c(c5)C5(c7ccccc7-c7ccccc75)c5ccccc5-6)cc4)c23)cc1] | 71 | 896.42 |
| | 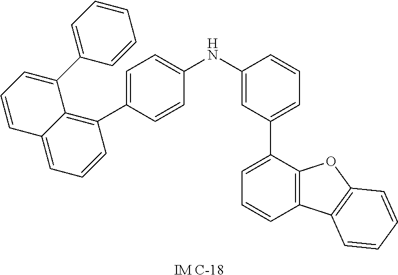 | 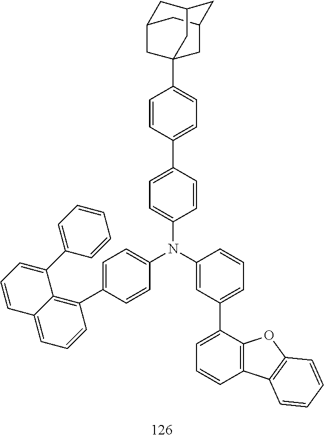 [SMILES: c1ccc(-c2cccc3cccc(-c4ccc(N(c5ccc(-c6ccc(C78CC9CC(CC(C9)C7)C8)cc6)cc5)c5cccc(-c6cccc7c6oc6ccccc67)c5)cc4)c23)cc1] | 79 | 824.38 |
| | 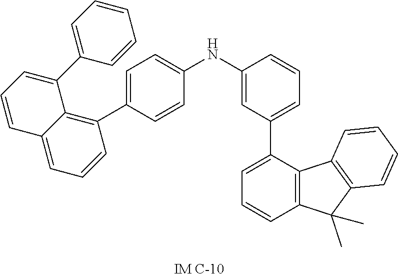 [SMILES: CC1(C)c2ccccc2-c2c(-c3cccc(Nc4ccc(-c5cccc6cccc(-c7ccccc7)c56)cc4)c3)cccc21] | 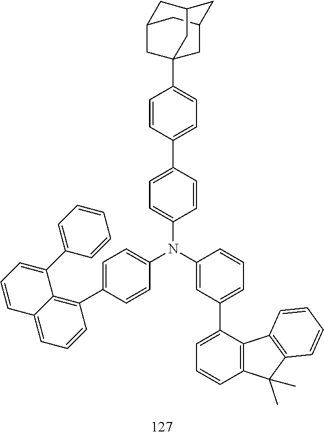 [SMILES: CC1(C)c2ccccc2-c2c(-c3cccc(N(c4ccc(-c5ccc(C67CC8CC(CC(C8)C6)C7)cc5)cc4)c4ccc(-c5cccc6cccc(-c7ccccc7)c56)cc4)c3)cccc21] | 75 | 850.43 |
| | 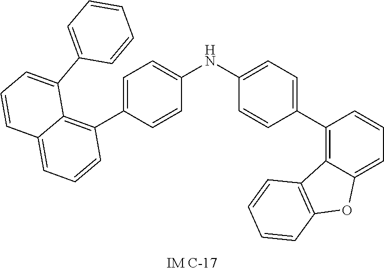 | 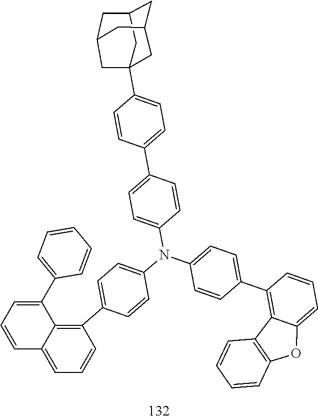 [SMILES: c1ccc(-c2cccc3cccc(-c4ccc(N(c5ccc(-c6ccc(C78CC9CC(CC(C9)C7)C8)cc6)cc5)c5ccc(-c6cccc7oc8ccccc8c67)cc5)cc4)c23)cc1] | 76 | 824.38 |
| | 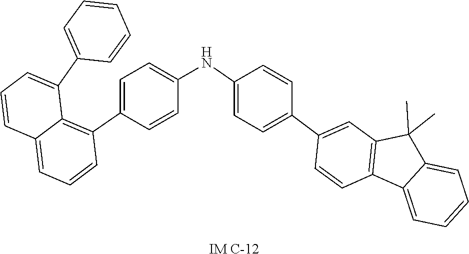 | 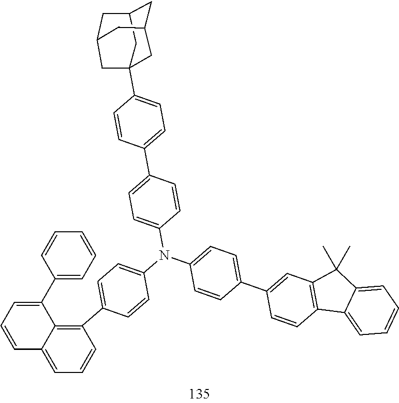 [SMILES: CC1(C)c2ccccc2-c2ccc(-c3ccc(N(c4ccc(-c5ccc(C67CC8CC(CC(C8)C6)C7)cc5)cc4)c4ccc(-c5cccc6cccc(-c7ccccc7)c56)cc4)cc3)cc21] | 78 | 850.43 |
| | 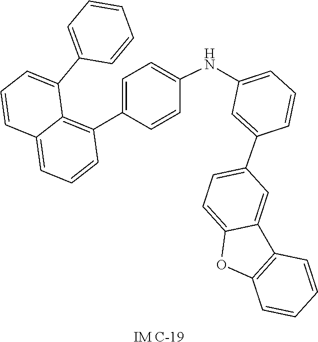 | 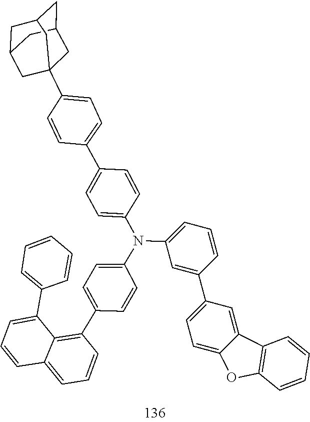 [SMILES: c1ccc(-c2cccc3cccc(-c4ccc(N(c5ccc(-c6ccc(C78CC9CC(CC(C9)C7)C8)cc6)cc5)c5cccc(-c6ccc7oc8ccccc8c7c6)c5)cc4)c23)cc1] | 81 | 824.38 |
| | 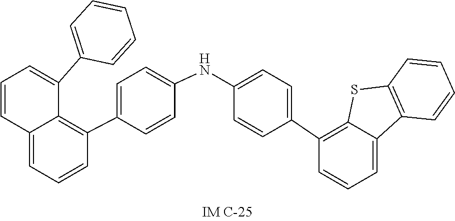 [SMILES: c1ccc(-c2cccc3cccc(-c4ccc(Nc5ccc(-c6cccc7c6sc6ccccc67)cc5)cc4)c23)cc1] | 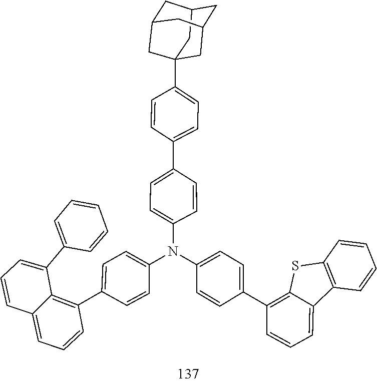 [SMILES: c1ccc(-c2cccc3cccc(-c4ccc(N(c5ccc(-c6ccc(C78CC9CC(CC(C9)C7)C8)cc6)cc5)c5ccc(-c6cccc7c6sc6ccccc67)cc5)cc4)c23)cc1] | 74 | 840.36 |
| | 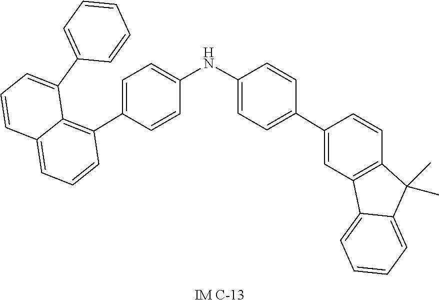 | 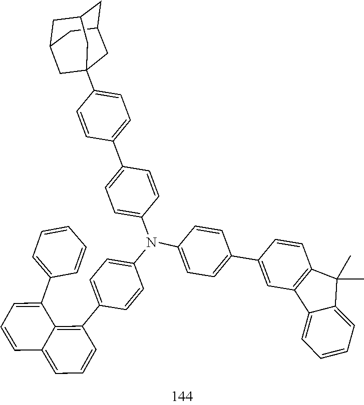 [SMILES: CC1(C)c2ccccc2-c2cc(-c3ccc(N(c4ccc(-c5ccc(C67CC8CC(CC(C8)C6)C7)cc5)cc4)c4ccc(-c5cccc6cccc(-c7ccccc7)c56)cc4)cc3)ccc21] | 71 | 850.43 |
| | 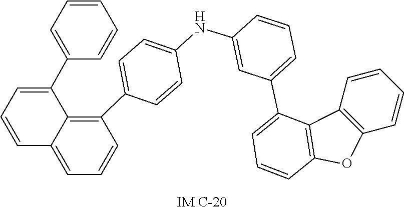 | 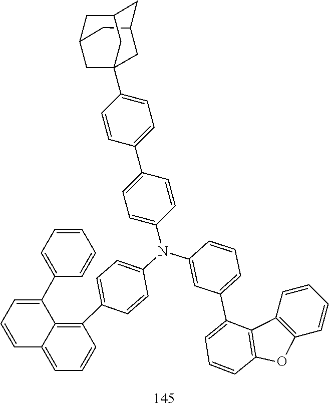 [SMILES: c1ccc(-c2cccc3cccc(-c4ccc(N(c5ccc(-c6ccc(C78CC9CC(CC(C9)C7)C8)cc6)cc5)c5cccc(-c6cccc7oc8ccccc8c67)c5)cc4)c23)cc1] | 75 | 824.38 |
| | 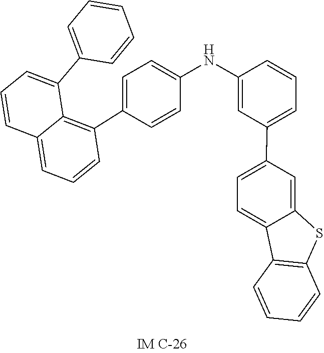 [SMILES: c1ccc(-c2cccc3cccc(-c4ccc(Nc5cccc(-c6ccc7c(c6)sc6ccccc67)c5)cc4)c23)cc1] | 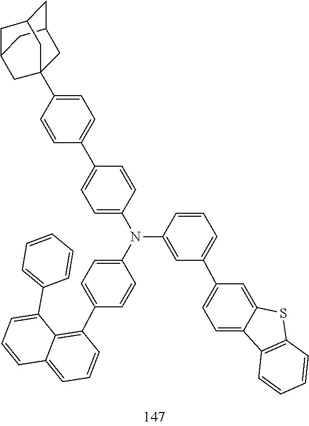 [SMILES: c1ccc(-c2cccc3cccc(-c4ccc(N(c5ccc(-c6ccc(C78CC9CC(CC(C9)C7)C8)cc6)cc5)c5cccc(-c6ccc7c(c6)sc6ccccc67)c5)cc4)c23)cc1] | 76 | 840.36 |
| | 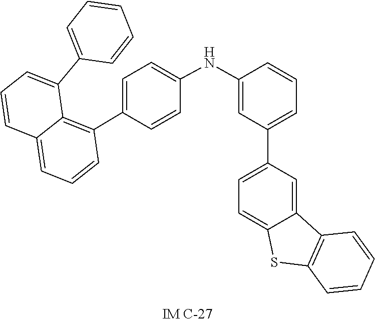 [SMILES: c1ccc(-c2cccc3cccc(-c4ccc(Nc5cccc(-c6ccc7sc8ccccc8c7c6)c5)cc4)c23)cc1] | 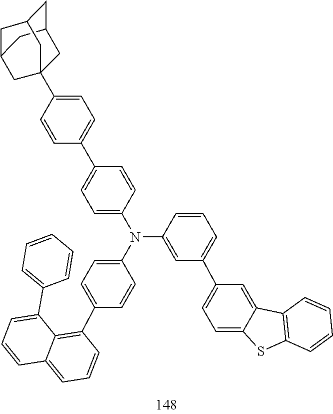 [SMILES: c1ccc(-c2cccc3cccc(-c4ccc(N(c5ccc(-c6ccc(C78CC9CC(CC(C9)C7)C8)cc6)cc5)c5cccc(-c6ccc7sc8ccccc8c7c6)c5)cc4)c23)cc1] | 79 | 840.36 |
| | 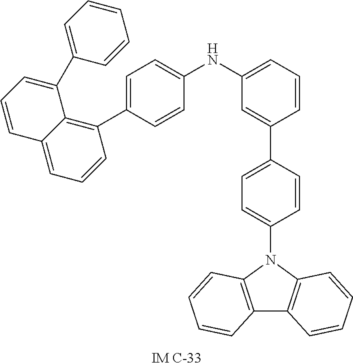 | 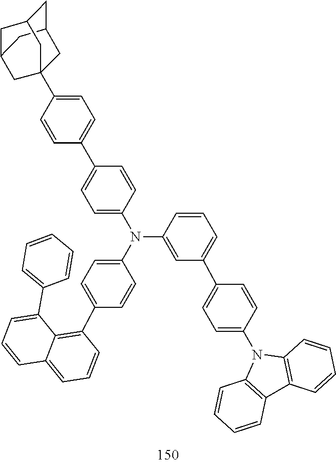 [SMILES: c1ccc(-c2cccc3cccc(-c4ccc(N(c5ccc(-c6ccc(C78CC9CC(CC(C9)C7)C8)cc6)cc5)c5cccc(-c6ccc(-n7c8ccccc8c8ccccc87)cc6)c5)cc4)c23)cc1] | 59 | 899.43 |
| 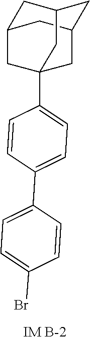 [SMILES: Brc1ccc(-c2ccc(C34CC5CC(CC(C5)C3)C4)cc2)cc1] | 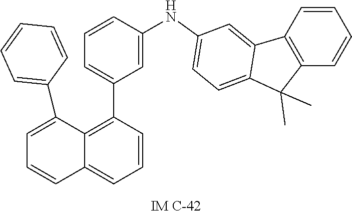 | 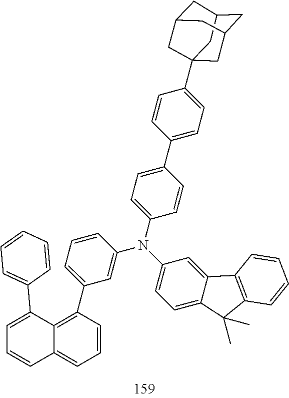 [SMILES: CC1(C)c2ccccc2-c2cc(N(c3ccc(-c4ccc(C56CC7CC(CC(C7)C5)C6)cc4)cc3)c3cccc(-c4cccc5cccc(-c6ccccc6)c45)c3)ccc21] | 76 | 774.40 |
| | 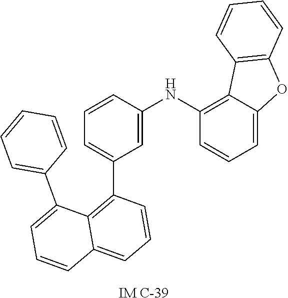 | 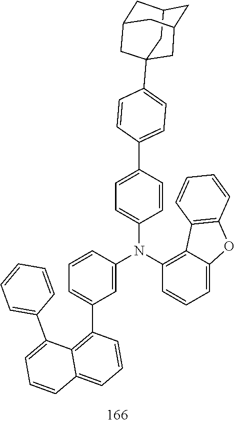 [SMILES: c1ccc(-c2cccc3cccc(-c4cccc(N(c5ccc(-c6ccc(C78CC9CC(CC(C9)C7)C8)cc6)cc5)c5cccc6oc7ccccc7c56)c4)c23)cc1] | 51 | 748.35 |
| | 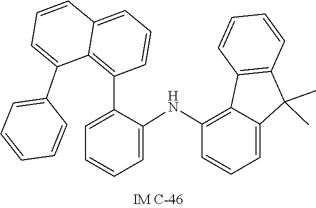 [SMILES: CC1(C)c2ccccc2-c2c(Nc3ccccc3-c3cccc4cccc(-c5ccccc5)c34)cccc21] | 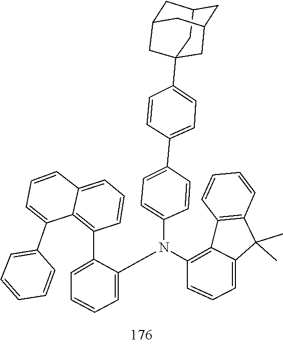 [SMILES: CC1(C)c2ccccc2-c2c(N(c3ccc(-c4ccc(C56CC7CC(CC(C7)C5)C6)cc4)cc3)c3ccccc3-c3cccc4cccc(-c5ccccc5)c34)cccc21] | 32 | 774.40 |
| 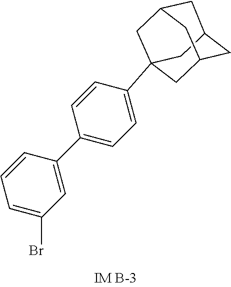 [SMILES: Brc1cccc(-c2ccc(C34CC5CC(CC(C5)C3)C4)cc2)c1] | 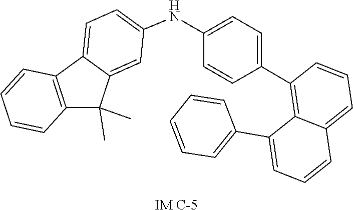 | 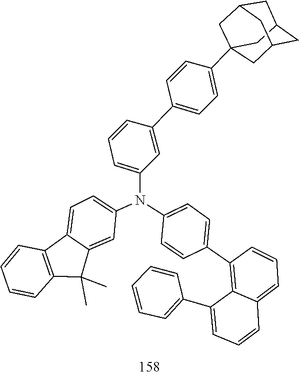 [SMILES: CC1(C)c2ccccc2-c2ccc(N(c3ccc(-c4cccc5cccc(-c6ccccc6)c45)cc3)c3cccc(-c4ccc(C56CC7CC(CC(C7)C5)C6)cc4)c3)cc21] | 58 | 774.40 |
| 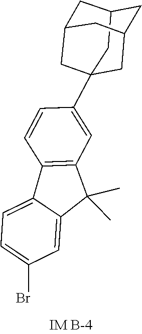 [SMILES: CC1(C)c2cc(Br)ccc2-c2ccc(C34CC5CC(CC(C5)C3)C4)cc21] | 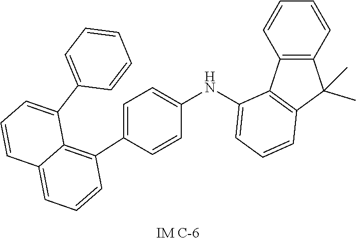 | 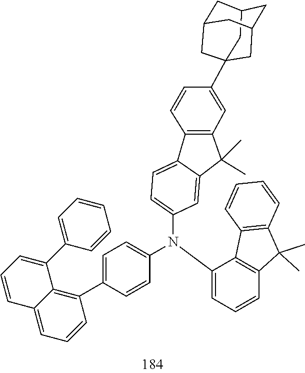 [SMILES: CC1(C)c2cc(N(c3ccc(-c4cccc5cccc(-c6ccccc6)c45)cc3)c3cccc4c3-c3ccccc3C4(C)C)ccc2-c2ccc(C34CC5CC(CC(C5)C3)C4)cc21] | 68 | 814.43 |
| 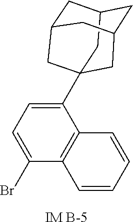 | 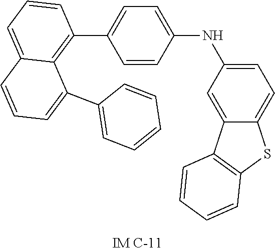 [SMILES: c1ccc(-c2cccc3cccc(-c4ccc(Nc5ccc6sc7ccccc7c6c5)cc4)c23)cc1] | 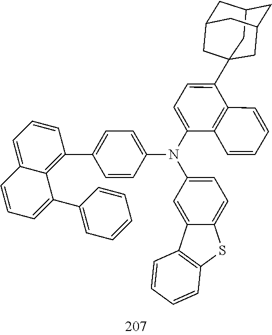 [SMILES: c1ccc(-c2cccc3cccc(-c4ccc(N(c5ccc6sc7ccccc7c6c5)c5ccc(C67CC8CC(CC(C8)C6)C7)c6ccccc56)cc4)c23)cc1] | 64 | 738.31 |
| 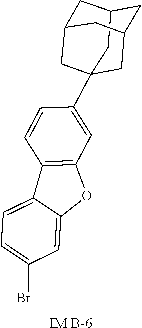 | 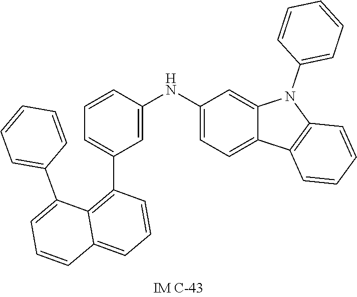 [SMILES: c1ccc(-c2cccc3cccc(-c4cccc(Nc5ccc6c7ccccc7n(-c7ccccc7)c6c5)c4)c23)cc1] | 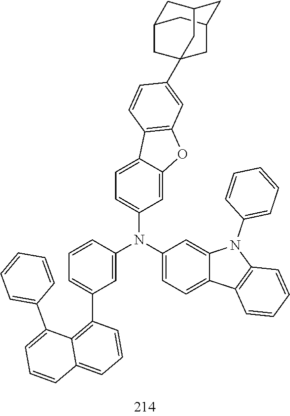 [SMILES: c1ccc(-c2cccc3cccc(-c4cccc(N(c5ccc6c(c5)oc5cc(C78CC9CC(CC(C9)C7)C8)ccc56)c5ccc6c7ccccc7n(-c7ccccc7)c6c5)c4)c23)cc1] | 73 | 837.38 |
| 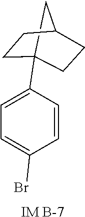 | 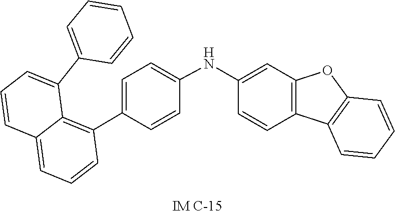 | 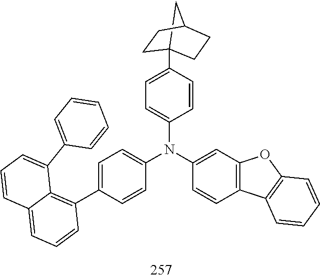 [SMILES: c1ccc(-c2cccc3cccc(-c4ccc(N(c5ccc(C67CCC(CC6)C7)cc5)c5ccc6c(c5)oc5ccccc56)cc4)c23)cc1] | 71 | 632.29 |
| | 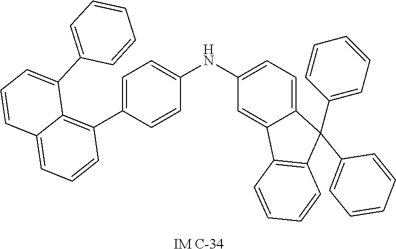 | 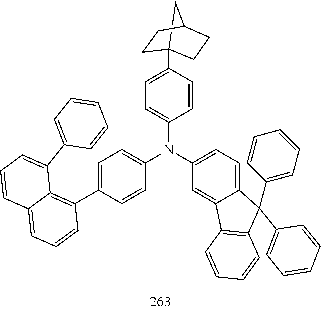 [SMILES: c1ccc(-c2cccc3cccc(-c4ccc(N(c5ccc(C67CCC(CC6)C7)cc5)c5ccc6c(c5)-c5ccccc5C6(c5ccccc5)c5ccccc5)cc4)c23)cc1] | 73 | 782.37 |
| | 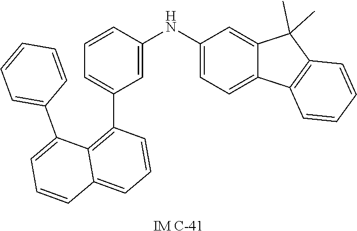 | 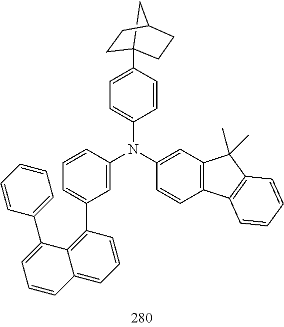 [SMILES: CC1(C)c2ccccc2-c2ccc(N(c3ccc(C45CCC(CC4)C5)cc3)c3cccc(-c4cccc5cccc(-c6ccccc6)c45)c3)cc21] | 69 | 658.34 |
| 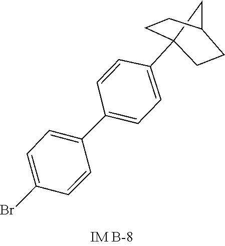 [SMILES: Brc1ccc(-c2ccc(C34CCC(CC3)C4)cc2)cc1] | 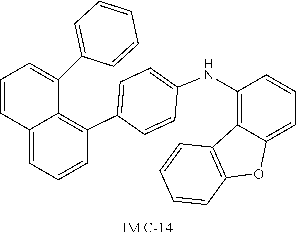 | 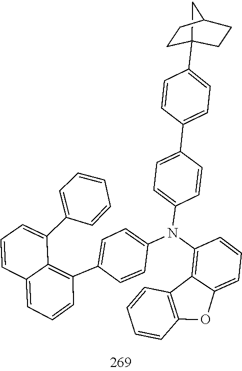 [SMILES: c1ccc(-c2cccc3cccc(-c4ccc(N(c5ccc(-c6ccc(C78CCC(CC7)C8)cc6)cc5)c5cccc6oc7ccccc7c56)cc4)c23)cc1] | 61 | 708.32 |
| | 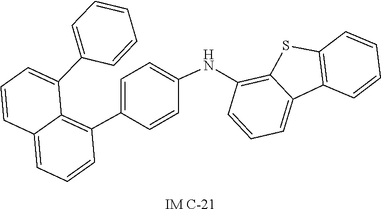 | 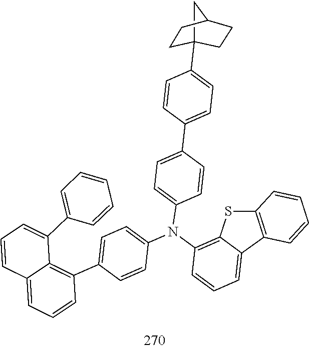 [SMILES: c1ccc(-c2cccc3cccc(-c4ccc(N(c5ccc(-c6ccc(C78CCC(CC7)C8)cc6)cc5)c5cccc6c5sc5ccccc56)cc4)c23)cc1] | 60 | 724.30 |
| | 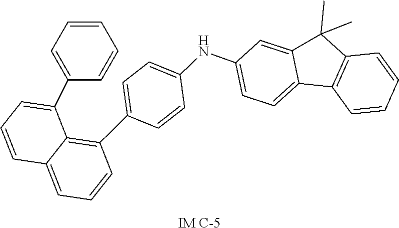 | 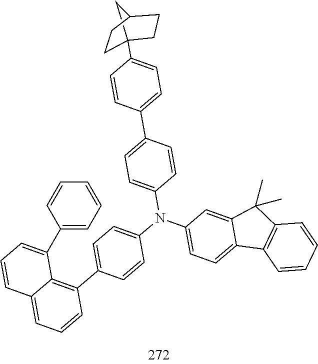 [SMILES: CC1(C)c2ccccc2-c2ccc(N(c3ccc(-c4ccc(C56CCC(CC5)C6)cc4)cc3)c3ccc(-c4cccc5cccc(-c6ccccc6)c45)cc3)cc21] | 74 | 734.37 |
| | 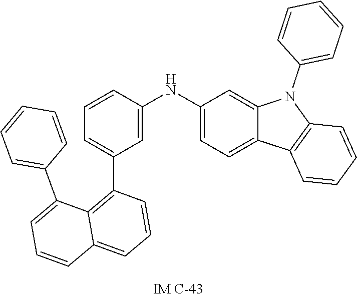 [SMILES: c1ccc(-c2cccc3cccc(-c4cccc(Nc5ccc6c7ccccc7n(-c7ccccc7)c6c5)c4)c23)cc1] | 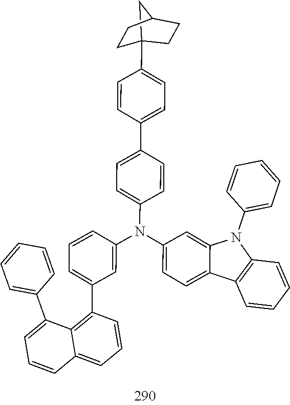 [SMILES: c1ccc(-c2cccc3cccc(-c4cccc(N(c5ccc(-c6ccc(C78CCC(CC7)C8)cc6)cc5)c5ccc6c7ccccc7n(-c7ccccc7)c6c5)c4)c23)cc1] | 67 | 783.37 |
| 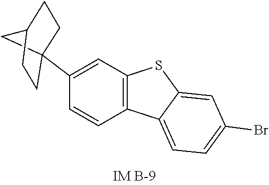 [SMILES: Brc1ccc2c(c1)sc1cc(C34CCC(CC3)C4)ccc12] | 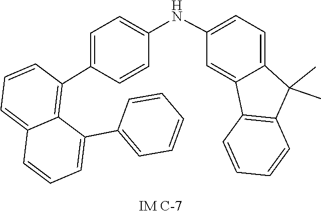 [SMILES: CC1(C)c2ccccc2-c2cc(Nc3ccc(-c4cccc5cccc(-c6ccccc6)c45)cc3)ccc21] | 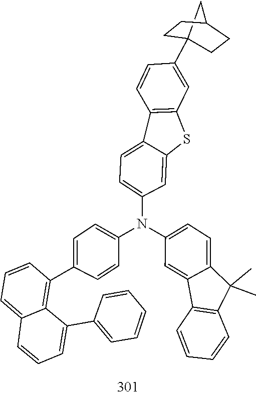 [SMILES: CC1(C)c2ccccc2-c2cc(N(c3ccc(-c4cccc5cccc(-c6ccccc6)c45)cc3)c3ccc4c(c3)sc3cc(C56CCC(CC5)C6)ccc34)ccc21] | 61 | 764.33 |
| 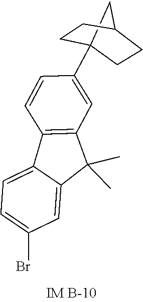 [SMILES: CC1(C)c2cc(Br)ccc2-c2ccc(C34CCC(CC3)C4)cc21] | 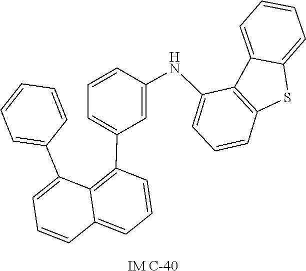 | 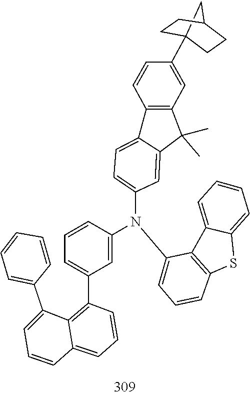 [SMILES: CC1(C)c2cc(N(c3cccc(-c4cccc5cccc(-c6ccccc6)c45)c3)c3cccc4sc5ccccc5c34)ccc2-c2ccc(C34CCC(CC3)C4)cc21] | 72 | 764.33 |
| 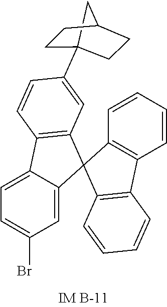 [SMILES: Brc1ccc2c(c1)C1(c3ccccc3-c3ccccc31)c1cc(C34CCC(CC3)C4)ccc1-2] | 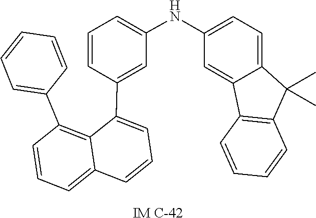 | 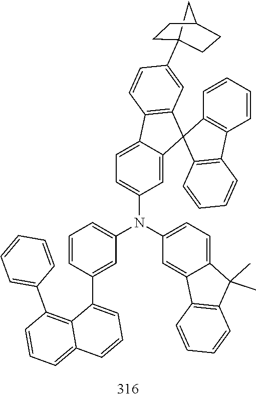 [SMILES: CC1(C)c2ccccc2-c2cc(N(c3cccc(-c4cccc5cccc(-c6ccccc6)c45)c3)c3ccc4c(c3)C3(c5ccccc5-c5ccccc53)c3cc(C56CCC(CC5)C6)ccc3-4)ccc21] | 43 | 896.42 |
| 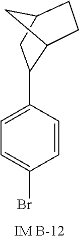 [SMILES: Brc1ccc(C2CC3CCC2C3)cc1] | 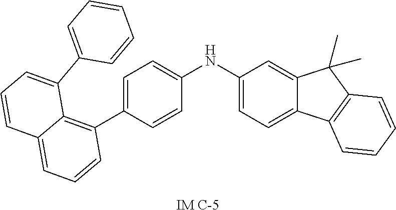 | 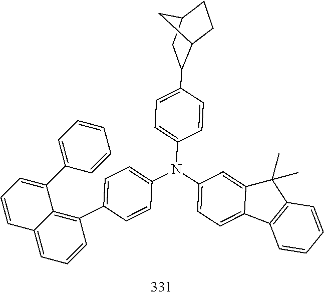 [SMILES: CC1(C)c2ccccc2-c2ccc(N(c3ccc(-c4cccc5cccc(-c6ccccc6)c45)cc3)c3ccc(C4CC5CCC4C5)cc3)cc21] | 73 | 658.34 |
| | 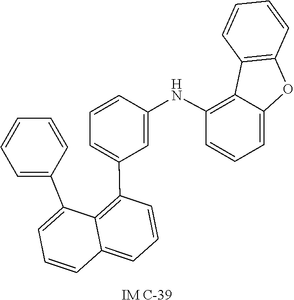 | 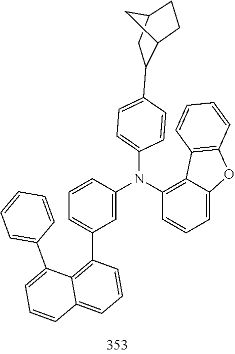 [SMILES: c1ccc(-c2cccc3cccc(-c4cccc(N(c5ccc(C6CC7CCC6C7)cc5)c5cccc6oc7ccccc7c56)c4)c23)cc1] | 61 | 632.29 |
| | 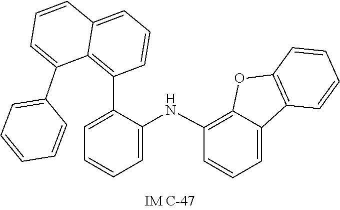 [SMILES: c1ccc(-c2cccc3cccc(-c4ccccc4Nc4cccc5c4oc4ccccc45)c23)cc1] | 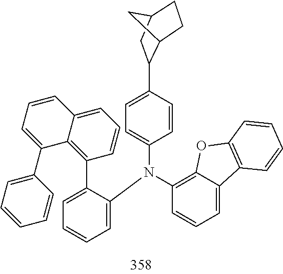 [SMILES: c1ccc(-c2cccc3cccc(-c4ccccc4N(c4ccc(C5CC6CCC5C6)cc4)c4cccc5c4oc4ccccc45)c23)cc1] | 42 | 632.29 |
| 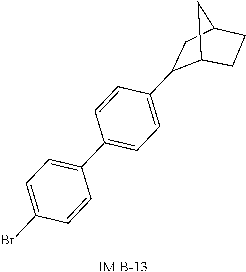 | 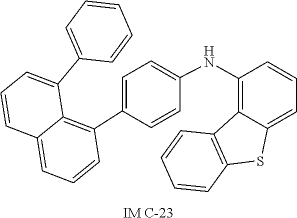 | 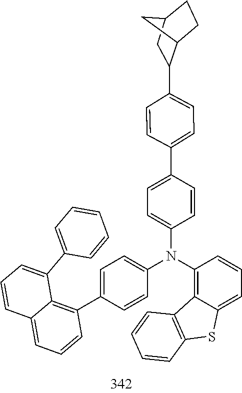 [SMILES: c1ccc(-c2cccc3cccc(-c4ccc(N(c5ccc(-c6ccc(C7CC8CCC7C8)cc6)cc5)c5cccc6sc7ccccc7c56)cc4)c23)cc1] | 67 | 724.30 |
| | 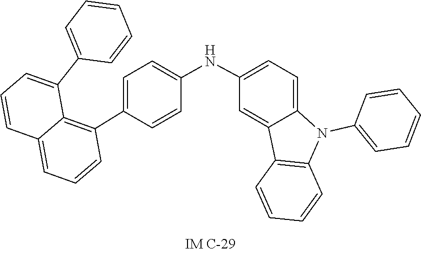 [SMILES: c1ccc(-c2cccc3cccc(-c4ccc(Nc5ccc6c(c5)c5ccccc5n6-c5ccccc5)cc4)c23)cc1] | 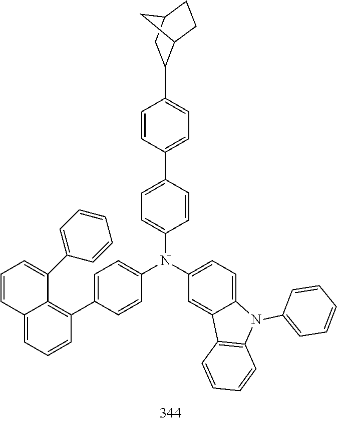 [SMILES: c1ccc(-c2cccc3cccc(-c4ccc(N(c5ccc(-c6ccc(C7CC8CCC7C8)cc6)cc5)c5ccc6c(c5)c5ccccc5n6-c5ccccc5)cc4)c23)cc1] | 71 | 783.37 |
| | 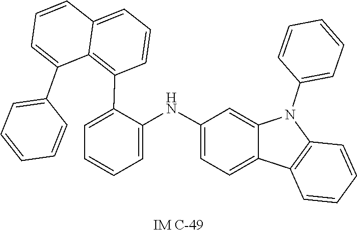 | 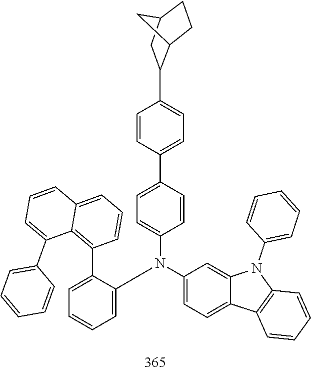 [SMILES: c1ccc(-c2cccc3cccc(-c4ccccc4N(c4ccc(-c5ccc(C6CC7CCC6C7)cc5)cc4)c4ccc5c6ccccc6n(-c6ccccc6)c5c4)c23)cc1] | 43 | 783.37 |
| 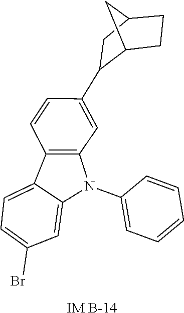 | 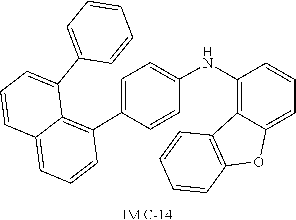 | 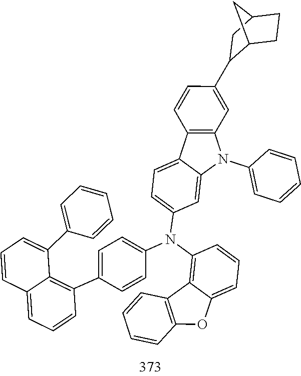 [SMILES: c1ccc(-c2cccc3cccc(-c4ccc(N(c5ccc6c7ccc(C8CC9CCC8C9)cc7n(-c7ccccc7)c6c5)c5cccc6oc7ccccc7c56)cc4)c23)cc1] | 54 | 797.35 |
| 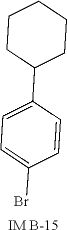 | 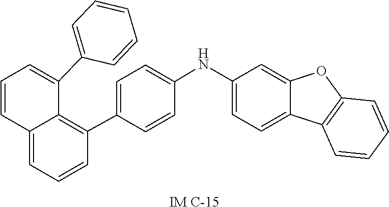 | 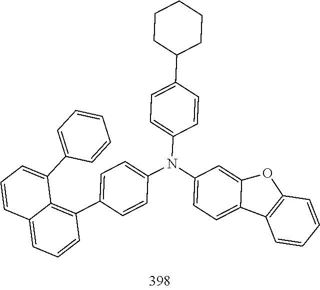 [SMILES: c1ccc(-c2cccc3cccc(-c4ccc(N(c5ccc(C6CCCCC6)cc5)c5ccc6c(c5)oc5ccccc56)cc4)c23)cc1] | 76 | 620.29 |
| | 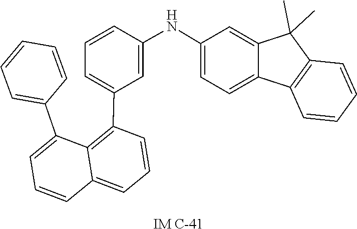 | 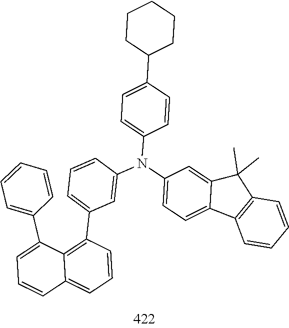 [SMILES: CC1(C)c2ccccc2-c2ccc(N(c3ccc(C4CCCCC4)cc3)c3cccc(-c4cccc5cccc(-c6ccccc6)c45)c3)cc21] | 77 | 646.34 |
| 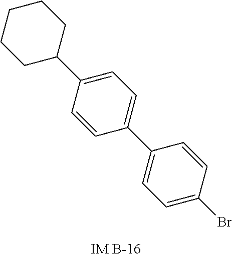 | 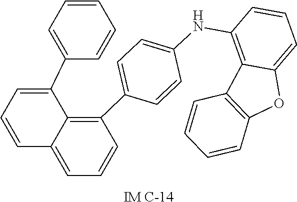 | 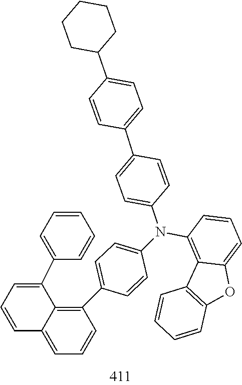 [SMILES: c1ccc(-c2cccc3cccc(-c4ccc(N(c5ccc(-c6ccc(C7CCCCC7)cc6)cc5)c5cccc6oc7ccccc7c56)cc4)c23)cc1] | 75 | 696.32 |
| | 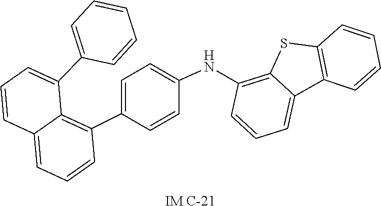 | 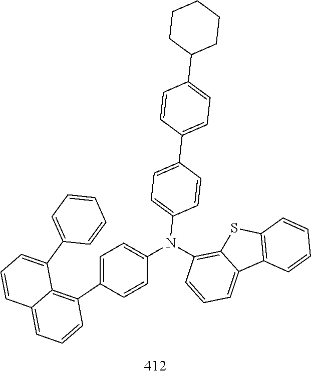 [SMILES: c1ccc(-c2cccc3cccc(-c4ccc(N(c5ccc(-c6ccc(C7CCCCC7)cc6)cc5)c5cccc6c5sc5ccccc56)cc4)c23)cc1] | 57 | 712.30 |
| | 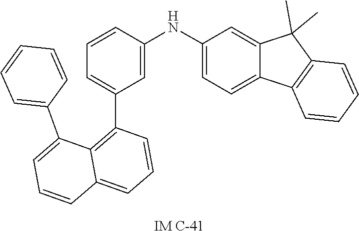 | 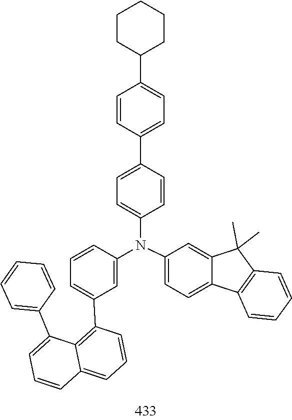 [SMILES: CC1(C)c2ccccc2-c2ccc(N(c3ccc(-c4ccc(C5CCCCC5)cc4)cc3)c3cccc(-c4cccc5cccc(-c6ccccc6)c45)c3)cc21] | 71 | 722.37 |
| | 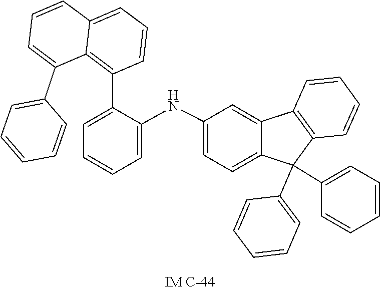 [SMILES: c1ccc(-c2cccc3cccc(-c4ccccc4Nc4ccc5c(c4)-c4ccccc4C5(c4ccccc4)c4ccccc4)c23)cc1] | 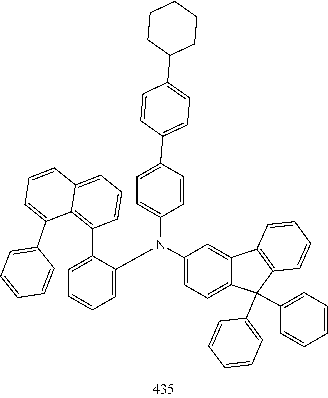 [SMILES: c1ccc(-c2cccc3cccc(-c4ccccc4N(c4ccc(-c5ccc(C6CCCCC6)cc5)cc4)c4ccc5c(c4)-c4ccccc4C5(c4ccccc4)c4ccccc4)c23)cc1] | 58 | 846.40 |
| 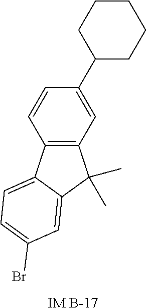 | 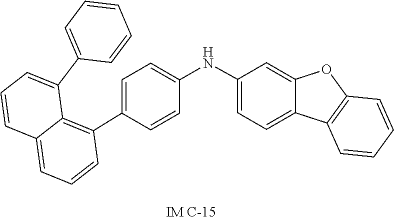 | 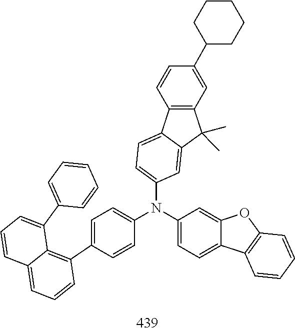 [SMILES: CC1(C)c2cc(C3CCCCC3)ccc2-c2ccc(N(c3ccc(-c4cccc5cccc(-c6ccccc6)c45)cc3)c3ccc4c(c3)oc3ccccc34)cc21] | 68 | 736.35 |
| 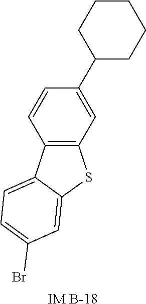 | 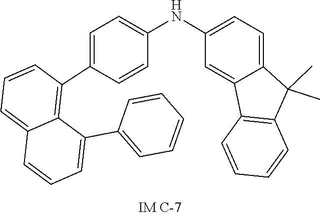 [SMILES: CC1(C)c2ccccc2-c2cc(Nc3ccc(-c4cccc5cccc(-c6ccccc6)c45)cc3)ccc21] | 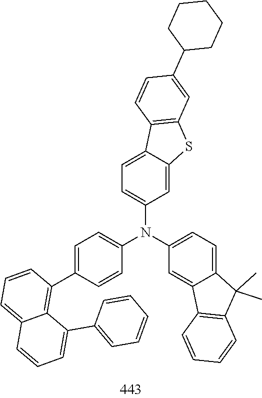 [SMILES: CC1(C)c2ccccc2-c2cc(N(c3ccc(-c4cccc5cccc(-c6ccccc6)c45)cc3)c3ccc4c(c3)sc3cc(C5CCCCC5)ccc34)ccc21] | 67 | 752.33 |
| 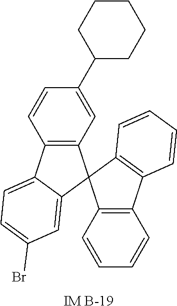 | 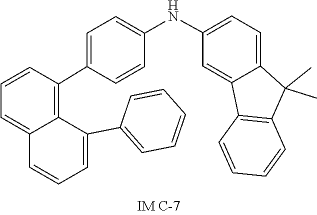 [SMILES: CC1(C)c2ccccc2-c2cc(Nc3ccc(-c4cccc5cccc(-c6ccccc6)c45)cc3)ccc21] | 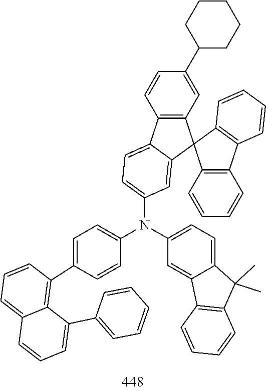 [SMILES: CC1(C)c2ccccc2-c2cc(N(c3ccc(-c4cccc5cccc(-c6ccccc6)c45)cc3)c3ccc4c(c3)C3(c5ccccc5-c5ccccc53)c3cc(C5CCCCC5)ccc3-4)ccc21] | 43 | 884.42 |

NMR data for some compounds are as follows:

| | |
|---|---|
| Compound | ¹H-NMR (400 MHz, CD₂Cl₂): δ ppm 8.34 (d, 1H), 8.21 (d, 2H), 8.03-7.99 (m, 4H), |
| 7 | 7.96-7.73 (m, 13H), 7.53-7.25 (m, 2H), 7.14 (m, 3H), 7.01 (d, 1H), 2.12 (s, 3H), |
| | 1.95 (s, 6H), 1.82-1.76 (m, 6H), 1.61 (s, 6H). |
| Compound | ¹H-NMR (400 MHZ, CD₂Cl₂): δ ppm 8.48 (t, 2H), 8.38 (d, 2H), 8.03-7.73 (m, 11H), |
| 257 | 7.58-7.55 (m, 3H), 7.41-7.38 (d, 1H), 7.31 (d, 5H), 7.02 (d, 2H), 2.32 (s, 1H), 2.06- |
| | 1.81 (m, 10H). |
| Compound | ¹H-NMR (400 MHZ, CD₂Cl₂): δ ppm 8.34-8.18 (m, 3H), 7.93-7.36 (m, 22H), 7.23- |
| 433 | 7.15 (m, 2H), 7.02-6.91 (m, 3H), 2.82-2.76 (m, 1H), 1.61 (s, 6H), 1.55-1.26 (m, |
| | 10H). |

### DEVICE EXAMPLES

### Example 1

Blue Organic Electroluminescent Device

An organic electroluminescent device was manufactured by the following process: an ITO substrate (manufactured by Corning) with a thickness of 1500 Å was cut into a dimension of 40 mm (length)×40 mm (width)×0.7 mm (thickness) to be prepared into an experimental substrate with a cathode, an anode and an insulation layer pattern by using a photoetching process, and surface treatment was performed by using ultraviolet ozone and O₂:N₂ plasma to increase the work function of the anode (the experimental substrate) and remove scum.

F4-TCNQ was vacuum evaporated on the experimental substrate (the anode) to form a hole injection layer (HIL) with a thickness of 100 Å, and NPB was evaporated on the hole injection layer to form a hole transport layer (HTL) with a thickness of 1200 Å.

A compound 2 was vacuum evaporated on the hole transport layer to form an electron blocking layer (EBL) with a thickness of 100 Å.

BH-01 and BD-01 were co-evaporated on the electron blocking layer at a film thickness ratio of 98%:2% to form a blue organic light-emitting layer (EML) with a thickness of 220 Å.

ET-06 and LiQ were evaporated on the organic light-emitting layer at a film thickness ratio of 1:1 to form an electron transport layer (ETL) with a thickness of 300 Å, Yb was evaporated on the electron transport layer to form an electron injection layer (EIL) with a thickness of 15 Å, and then magnesium (Mg) and silver (Ag) were vacuum evaporated on the electron injection layer at a film thickness ratio of 1:9 to form a cathode with a thickness of 120 Å.

In addition, CP-05 was evaporated on the cathode to form an organic capping layer (CPL) with a thickness of 650 Å, thus completing the manufacture of the organic electroluminescent device.

### Examples 2-78

An organic electroluminescent device was manufactured by the same method as that in Example 1, except that the remaining compounds described in Table 7 were used instead of the compound 2 when the electron blocking layer was formed.

### Comparative Examples 1-4

In Comparative examples 1-4, an organic electroluminescent device was manufactured by the same method as that in Example 1, except that a compound A, a compound B, a compound C, and a compound D were respectively used instead of the compound 2 when the electron blocking layer was formed.

In the above Examples and Comparative examples, the structures of the used main materials are shown in Table 6.

TABLE 6

| | |
|---|---|
| 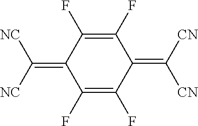 [SMILES: N#CC(C#N)=c1c(F)c(F)c(=C(C#N)C#N)c(F)c1F] | F4-TCNQ |
| 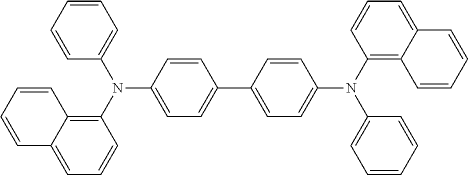 [SMILES: c1ccc(N(c2ccc(-c3ccc(N(c4ccccc4)c4cccc5ccccc45)cc3)cc2)c2cccc3ccccc23)cc1] | NPB |
| 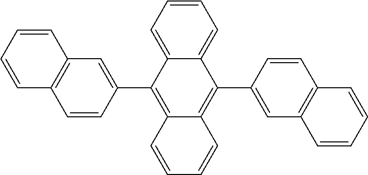 [SMILES: c1ccc2cc(-c3c4ccccc4c(-c4ccc5ccccc5c4)c4ccccc34)ccc2c1] | BH-01 |
| 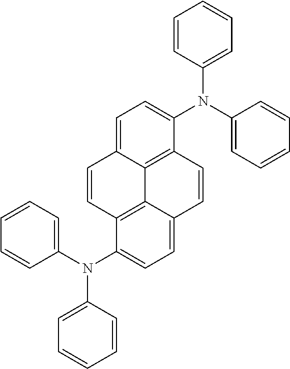 [SMILES: c1ccc(N(c2ccccc2)c2ccc3ccc4c(N(c5ccccc5)c5ccccc5)ccc5ccc2c3c54)cc1] | BD-01 |
| 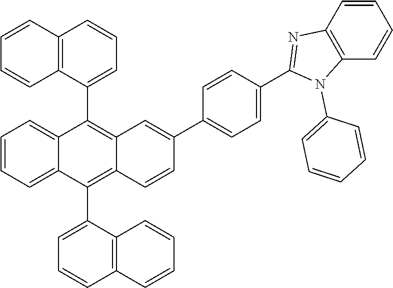 [SMILES: c1ccc(-n2c(-c3ccc(-c4ccc5c(-c6cccc7ccccc67)c6ccccc6c(-c6cccc7ccccc67)c5c4)cc3)nc3ccccc32)cc1] | ET-06 |
| 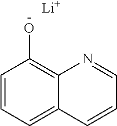 | LiQ |
| 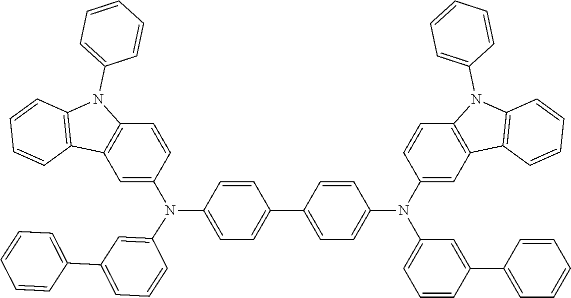 [SMILES: c1ccc(-c2cccc(N(c3ccc(-c4ccc(N(c5cccc(-c6ccccc6)c5)c5ccc6c(c5)c5ccccc5n6-c5ccccc5)cc4)cc3)c3ccc4c(c3)c3ccccc3n4-c3ccccc3)c2)cc1] | CP-05 |
| 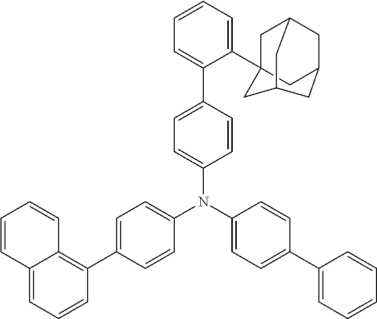 [SMILES: c1ccc(-c2ccc(N(c3ccc(-c4ccccc4C45CC6CC(CC(C6)C4)C5)cc3)c3ccc(-c4cccc5ccccc45)cc3)cc2)cc1] | Compound A |
| 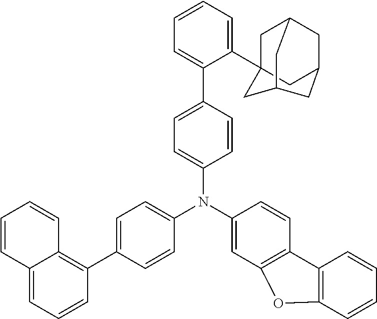 [SMILES: c1ccc(C23CC4CC(CC(C4)C2)C3)c(-c2ccc(N(c3ccc(-c4cccc5ccccc45)cc3)c3ccc4c(c3)oc3ccccc34)cc2)c1] | Compound B |
| 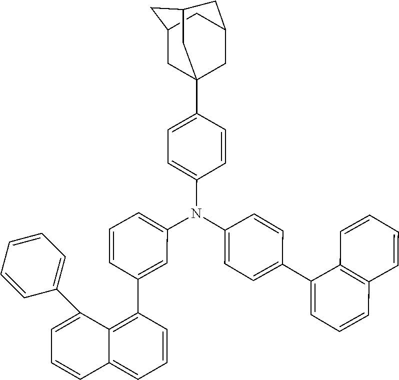 [SMILES: c1ccc(-c2cccc3cccc(-c4cccc(N(c5ccc(-c6cccc7ccccc67)cc5)c5ccc(C67CC8CC(CC(C8)C6)C7)cc5)c4)c23)cc1] | Compound C |
| 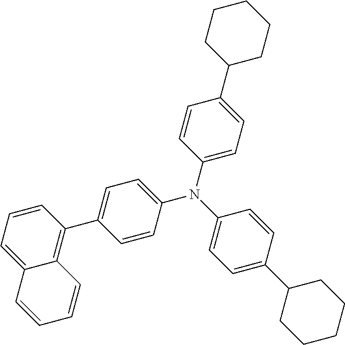 [SMILES: c1ccc2c(-c3ccc(N(c4ccc(C5CCCCC5)cc4)c4ccc(C5CCCCC5)cc4)cc3)cccc2c1] | Compound D |

The properties of the organic electroluminescent devices manufactured in the Examples and Comparative examples are shown in Table 7, where the properties of the devices were analyzed under a condition of 20 mA/cm².

TABLE 7

| | | Driving | | | | External | |
|---|---|---|---|---|---|---|---|
| | | voltage | Luminous | Power | Chromaticity | quantum | |
| | | Volt | efficiency | efficiency | coordinate | efficiency, | T95 |
| Example | EBL | (V) | Cd/A | (lm/W) | CIE-x, CIE-y | EQE% | (h) |
| Example 1 | Compound | 3.97 | 6.55 | 5.18 | 0.14, 0.05 | 13.47 | 252 |
| | 2 | | | | | | |
| Example 2 | Compound | 3.95 | 6.51 | 5.18 | 0.14, 0.05 | 13.39 | 259 |
| | 3 | | | | | | |
| Example 3 | Compound | 3.88 | 6.80 | 5.51 | 0.14, 0.05 | 13.99 | 256 |
| | 6 | | | | | | |
| Example 4 | Compound | 3.92 | 6.59 | 5.28 | 0.14, 0.05 | 13.56 | 259 |
| | 7 | | | | | | |
| Example 5 | Compound | 3.88 | 6.46 | 5.23 | 0.14, 0.05 | 13.29 | 262 |
| | 10 | | | | | | |
| Example 6 | Compound | 3.93 | 6.70 | 5.36 | 0.14, 0.05 | 13.78 | 252 |
| | 14 | | | | | | |
| Example 7 | Compound | 3.97 | 6.70 | 5.30 | 0.14, 0.05 | 13.78 | 260 |
| | 17 | | | | | | |
| Example 8 | Compound | 3.89 | 6.77 | 5.47 | 0.14, 0.05 | 13.93 | 263 |
| | 22 | | | | | | |
| Example 9 | Compound | 3.97 | 6.78 | 5.37 | 0.14, 0.05 | 13.95 | 263 |
| | 24 | | | | | | |
| Example 10 | Compound | 3.87 | 6.55 | 5.32 | 0.14, 0.05 | 13.47 | 263 |
| | 27 | | | | | | |
| Example 11 | Compound | 3.91 | 6.83 | 5.49 | 0.14, 0.05 | 14.05 | 264 |
| | 29 | | | | | | |
| Example 12 | Compound | 3.94 | 6.74 | 5.37 | 0.14, 0.05 | 13.86 | 269 |
| | 32 | | | | | | |
| Example 13 | Compound | 3.91 | 6.84 | 5.50 | 0.14, 0.05 | 14.07 | 259 |
| | 35 | | | | | | |
| Example 14 | Compound | 3.89 | 6.45 | 5.21 | 0.14, 0.05 | 13.27 | 268 |
| | 37 | | | | | | |
| Example 15 | Compound | 3.90 | 6.90 | 5.56 | 0.14, 0.05 | 14.19 | 256 |
| | 42 | | | | | | |
| Example 16 | Compound | 3.87 | 6.90 | 5.60 | 0.14, 0.05 | 14.19 | 257 |
| | 45 | | | | | | |
| Example 17 | Compound | 3.91 | 6.80 | 5.46 | 0.14, 0.05 | 13.99 | 255 |
| | 47 | | | | | | |
| Example 18 | Compound | 3.92 | 6.79 | 5.44 | 0.14, 0.05 | 13.97 | 263 |
| | 54 | | | | | | |
| Example 19 | Compound | 3.89 | 6.47 | 5.23 | 0.14, 0.05 | 13.31 | 265 |
| | 64 | | | | | | |
| Example 20 | Compound | 3.87 | 6.73 | 5.46 | 0.14, 0.05 | 13.84 | 265 |
| | 74 | | | | | | |
| Example 21 | Compound | 3.89 | 6.78 | 5.48 | 0.14, 0.05 | 13.95 | 254 |
| | 82 | | | | | | |
| Example 22 | Compound | 3.90 | 6.49 | 5.23 | 0.14, 0.05 | 13.35 | 260 |
| | 93 | | | | | | |
| Example 23 | Compound | 3.94 | 6.61 | 5.27 | 0.14, 0.05 | 13.60 | 250 |
| | 94 | | | | | | |
| Example 24 | Compound | 3.90 | 6.64 | 5.35 | 0.14, 0.05 | 13.66 | 260 |
| | 97 | | | | | | |
| Example 25 | Compound | 3.89 | 6.80 | 5.49 | 0.14, 0.05 | 13.99 | 255 |
| | 98 | | | | | | |
| Example 26 | Compound | 3.95 | 6.59 | 5.24 | 0.14, 0.05 | 13.56 | 265 |
| | 101 | | | | | | |
| Example 27 | Compound | 3.87 | 6.63 | 5.38 | 0.14, 0.05 | 13.64 | 251 |
| | 102 | | | | | | |
| Example 28 | Compound | 3.95 | 6.53 | 5.19 | 0.14, 0.05 | 13.43 | 265 |
| | 105 | | | | | | |
| Example 29 | Compound | 3.87 | 6.57 | 5.33 | 0.14, 0.05 | 13.51 | 261 |
| | 107 | | | | | | |
| Example 30 | Compound | 3.91 | 6.63 | 5.33 | 0.14, 0.05 | 13.64 | 261 |
| | 108 | | | | | | |
| Example 31 | Compound | 3.87 | 6.77 | 5.50 | 0.14, 0.05 | 13.93 | 259 |
| | 114 | | | | | | |
| Example 32 | Compound | 3.98 | 6.72 | 5.30 | 0.14, 0.05 | 13.82 | 252 |
| | 117 | | | | | | |
| Example 33 | Compound | 3.95 | 6.66 | 5.30 | 0.14, 0.05 | 13.70 | 254 |
| | 120 | | | | | | |
| Example 34 | Compound | 3.97 | 6.52 | 5.16 | 0.14, 0.05 | 13.41 | 258 |
| | 123 | | | | | | |
| Example 35 | Compound | 3.90 | 6.78 | 5.46 | 0.14, 0.05 | 13.95 | 262 |
| | 126 | | | | | | |
| Example 36 | Compound | 3.89 | 6.69 | 5.40 | 0.14, 0.05 | 13.76 | 261 |
| | 127 | | | | | | |
| Example 37 | Compound | 3.98 | 6.50 | 5.13 | 0.14, 0.05 | 13.37 | 265 |
| | 132 | | | | | | |
| Example 38 | Compound | 3.89 | 6.88 | 5.56 | 0.14, 0.05 | 14.15 | 269 |
| | 135 | | | | | | |
| Example 39 | Compound | 3.90 | 6.47 | 5.21 | 0.14, 0.05 | 13.31 | 254 |
| | 136 | | | | | | |
| Example 40 | Compound | 3.86 | 6.45 | 5.25 | 0.14, 0.05 | 13.27 | 258 |
| | 137 | | | | | | |
| Example 41 | Compound | 3.94 | 6.80 | 5.42 | 0.14, 0.05 | 13.99 | 269 |
| | 144 | | | | | | |
| Example 42 | Compound | 3.88 | 6.84 | 5.54 | 0.14, 0.05 | 14.07 | 261 |
| | 145 | | | | | | |
| Example 43 | Compound | 3.90 | 6.75 | 5.44 | 0.14, 0.05 | 13.88 | 255 |
| | 147 | | | | | | |
| Example 44 | Compound | 3.95 | 6.85 | 5.45 | 0.14, 0.05 | 14.09 | 260 |
| | 148 | | | | | | |
| Example 45 | Compound | 3.90 | 6.46 | 5.20 | 0.14, 0.05 | 13.29 | 257 |
| | 150 | | | | | | |
| Example 46 | Compound | 3.92 | 6.76 | 5.42 | 0.14, 0.05 | 13.91 | 254 |
| | 158 | | | | | | |
| Example 47 | Compound | 3.93 | 6.48 | 5.18 | 0.14, 0.05 | 13.33 | 262 |
| | 159 | | | | | | |
| Example 48 | Compound | 3.95 | 6.87 | 5.46 | 0.14, 0.05 | 14.13 | 266 |
| | 166 | | | | | | |
| Example 49 | Compound | 3.95 | 6.62 | 5.26 | 0.14, 0.05 | 13.62 | 263 |
| | 176 | | | | | | |
| Example 50 | Compound | 3.96 | 6.50 | 5.16 | 0.14, 0.05 | 13.37 | 268 |
| | 184 | | | | | | |
| Example 51 | Compound | 3.97 | 6.83 | 5.40 | 0.14, 0.05 | 14.05 | 266 |
| | 207 | | | | | | |
| Example 52 | Compound | 3.97 | 6.48 | 5.13 | 0.14, 0.05 | 13.33 | 255 |
| | 214 | | | | | | |
| Example 53 | Compound | 3.97 | 6.63 | 5.25 | 0.14, 0.05 | 13.64 | 223 |
| | 257 | | | | | | |
| Example 54 | Compound | 3.86 | 6.59 | 5.36 | 0.14, 0.05 | 13.56 | 229 |
| | 263 | | | | | | |
| Example 55 | Compound | 3.91 | 6.52 | 5.24 | 0.14, 0.05 | 13.41 | 227 |
| | 269 | | | | | | |
| Example 56 | Compound | 3.86 | 6.82 | 5.55 | 0.14, 0.05 | 14.03 | 212 |
| | 270 | | | | | | |
| Example 57 | Compound | 3.88 | 6.85 | 5.55 | 0.14, 0.05 | 14.09 | 219 |
| | 272 | | | | | | |
| Example 58 | Compound | 3.91 | 6.56 | 5.27 | 0.14, 0.05 | 13.49 | 217 |
| | 280 | | | | | | |
| Example 59 | Compound | 3.89 | 6.50 | 5.25 | 0.14, 0.05 | 13.37 | 226 |
| | 290 | | | | | | |
| Example 60 | Compound | 3.93 | 6.56 | 5.24 | 0.14, 0.05 | 13.49 | 215 |
| | 301 | | | | | | |
| Example 61 | Compound | 3.98 | 6.90 | 5.45 | 0.14, 0.05 | 14.19 | 222 |
| | 309 | | | | | | |
| Example 62 | Compound | 3.92 | 6.70 | 5.37 | 0.14, 0.05 | 13.78 | 215 |
| | 316 | | | | | | |
| Example 63 | Compound | 3.98 | 6.61 | 5.22 | 0.14, 0.05 | 13.60 | 222 |
| | 331 | | | | | | |
| Example 64 | Compound | 3.89 | 6.83 | 5.52 | 0.14, 0.05 | 14.05 | 210 |
| | 342 | | | | | | |
| Example 65 | Compound | 3.97 | 6.85 | 5.42 | 0.14, 0.05 | 14.09 | 211 |
| | 344 | | | | | | |
| Example 66 | Compound | 3.95 | 6.50 | 5.17 | 0.14, 0.05 | 13.37 | 210 |
| | 353 | | | | | | |
| Example 67 | Compound | 3.91 | 6.73 | 5.41 | 0.14, 0.05 | 13.84 | 227 |
| | 358 | | | | | | |
| Example 68 | Compound | 3.93 | 6.67 | 5.33 | 0.14, 0.05 | 13.72 | 221 |
| | 365 | | | | | | |
| Example 69 | Compound | 3.87 | 6.71 | 5.45 | 0.14, 0.05 | 13.80 | 230 |
| | 373 | | | | | | |
| Example 70 | Compound | 3.86 | 6.71 | 5.46 | 0.14, 0.05 | 13.80 | 217 |
| | 398 | | | | | | |
| Example 71 | Compound | 3.97 | 6.66 | 5.27 | 0.14, 0.05 | 13.70 | 229 |
| | 411 | | | | | | |
| Example 72 | Compound | 3.92 | 6.90 | 5.53 | 0.14, 0.05 | 14.19 | 225 |
| | 412 | | | | | | |
| Example 73 | Compound | 3.88 | 6.75 | 5.47 | 0.14, 0.05 | 13.88 | 212 |
| | 422 | | | | | | |
| Example 74 | Compound | 3.89 | 6.85 | 5.53 | 0.14, 0.05 | 14.09 | 216 |
| | 433 | | | | | | |
| Example 75 | Compound | 3.86 | 6.73 | 5.48 | 0.14, 0.05 | 13.84 | 216 |
| | 435 | | | | | | |
| Example 76 | Compound | 3.87 | 6.50 | 5.28 | 0.14, 0.05 | 13.37 | 218 |
| | 439 | | | | | | |
| Example 77 | Compound | 3.88 | 6.87 | 5.56 | 0.14, 0.05 | 14.13 | 230 |
| | 443 | | | | | | |
| Example 78 | Compound | 3.86 | 6.52 | 5.31 | 0.14, 0.05 | 13.41 | 222 |
| | 448 | | | | | | |
| Comparative | Compound | 4.34 | 5.50 | 4.08 | 0.14, 0.05 | 11.31 | 187 |
| example 1 | A | | | | | | |
| Comparative | Compound | 4.23 | 5.54 | 4.14 | 0.14, 0.05 | 11.40 | 190 |
| example 2 | B | | | | | | |
| Comparative | Compound | 4.15 | 5.69 | 4.31 | 0.14, 0.05 | 11.70 | 198 |
| example 3 | C | | | | | | |
| Comparative | Compound | 4.30 | 5.61 | 4.11 | 0.14, 0.05 | 11.57 | 192 |
| example 4 | D | | | | | | |

From the results of Table 7, it can be seen that Examples 1-78 in which the compounds were used as the electron blocking layer have the advantages that for the above organic electroluminescent devices manufactured by using the compounds as the electron blocking layer in the present disclosure, the driving voltage was reduced by at least 0.17 V, the luminous efficiency (Cd/A) was improved by at least 13.36%, the external quantum efficiency was improved by at least 13.42%, the service life was improved by at least 6.1%, and the service life can be improved by 82 h at most compared with device Comparative examples 1-4 corresponding to known compounds.

Preferred examples of the present disclosure have been described above in detail with reference to the accompanying drawings, but the present disclosure is not limited to the specific details in the above-described examples, and many simple modifications may be made to the technical solutions of the present disclosure within the technical idea of the present disclosure, and these simple modifications all fall within the scope of protection of the present disclosure.

In addition, it should be noted that various specific technical features described in the above specific examples may be combined in any suitable manner without contradiction, and the various possible combinations are not otherwise described in the present disclosure in order to avoid unnecessary repetition.

In addition, the various examples of the present disclosure can also be combined at will, as long as they do not violate the idea of the present disclosure, they should also be regarded as the contents disclosed in the present disclosure.

1. An organic compound, having a structure represented by a formula 1: 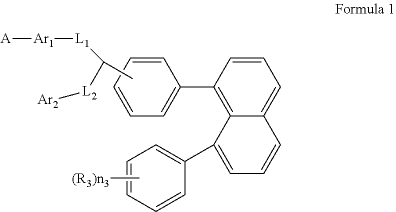
wherein in the formula 1, A is selected from adamantyl, norbornyl, or cyclohexyl;
Ar₁ is selected from substituted or unsubstituted aryl with 6 to 40 carbon atoms, and substituted or unsubstituted heteroaryl with 3 to 30 carbon atoms;
Ar₂ is selected from 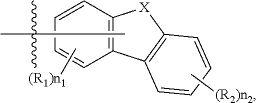
wherein X is selected from C(R₄R₅), N(R₆), O, S, or Si(R₇R₈), and [symbol] represents a chemical bond;
R₄, R₅, R₆, R₇ and R₈ are the same or different, and are each independently selected from hydrogen, alkyl with 1 to 5 carbon atoms, aryl with 6 to 12 carbon atoms, aryl with 7 to 17 carbon atoms substituted with alkyl with 1 to 5 carbon atoms, and heteroaryl with 3 to 12 carbon atoms; or, R₄ and R₅ form a saturated or unsaturated 3- to 15-membered ring; or, R₇ and R₈ form a saturated or unsaturated 3- to 15-membered ring;
R₁, R₂ and R₃ are the same or different, and are each independently selected from deuterium, cyano, a halogen group, alkyl with 1 to 5 carbon atoms, aryl with 6 to 12 carbon atoms, heteroaryl with 3 to 12 carbon atoms, and trialkylsilyl with 3 to 12 carbon atoms;
n₁ represents the number of R₁, n₂ represents the number of R₂, n₃ represents the number of R₃, n₁ and n₂ are each independently selected from 0, 1, 2, 3 or 4, and n₃ is selected from 0, 1, 2, 3, 4 or 5; and when n₁ is greater than 1, any two R₁ are the same or different; when n₂ is greater than 1, any two R₂ are the same or different; and when n₃ is greater than 1, any two R₃ are the same or different;
L₁ and L₂ are the same or different, and are each independently selected from a single bond, substituted or unsubstituted arylene with 6 to 30 carbon atoms, and substituted or unsubstituted heteroarylene with 3 to 30 carbon atoms; and
substituents in L₁, L₂, and Ar₁ are the same or different, and are respectively and independently selected from deuterium, cyano, a halogen group, alkyl with 1 to 5 carbon atoms, aryl with 6 to 12 carbon atoms, heteroaryl with 3 to 12 carbon atoms, and trialkylsilyl with 3 to 12 carbon atoms.

2. The organic compound according to claim 1, wherein the organic compound has a structure represented by a formula 1: 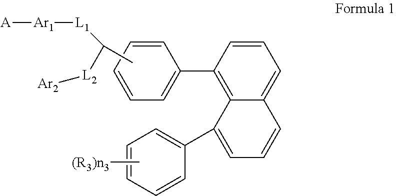
wherein in the formula 1, A is selected from adamantyl, norbornyl, or cyclohexyl;
Ar₁ is selected from substituted or unsubstituted aryl with 6 to 40 carbon atoms, and substituted or unsubstituted heteroaryl with 3 to 30 carbon atoms;
Ar₂ is selected from 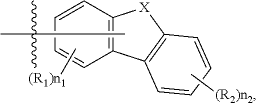
wherein X is selected from C(R₄R₅), N(R₆), O, S, or Si(R₇R₈), and [symbol] represents a chemical bond;
R₄, R₅, R₆, R₇ and R₈ are the same or different, and are each independently selected from alkyl with 1 to 5 carbon atoms, aryl having 6 to 12 carbon atoms, aryl with 7 to 17 carbon atoms substituted with alkyl with 1 to 5 carbon atoms, and heteroaryl with 3 to 12 carbon atoms; or, R₄ and R₅ form a saturated or unsaturated 3- to 15-membered ring; or, R₇ and R₈ form a saturated or unsaturated 3- to 15-membered ring;
R₁, R₂ and R₃ are the same or different, and are each independently selected from deuterium, cyano, a halogen group, alkyl with 1 to 5 carbon atoms, aryl with 6 to 12 carbon atoms, heteroaryl with 3 to 12 carbon atoms, and trialkylsilyl with 3 to 12 carbon atoms;
n₁ represents the number of R₁, n₂ represents the number of R₂, n₃ represents the number of R₃, n₁ and n₂ are each independently selected from 0, 1, 2, 3 or 4, and n₃ is selected from 0, 1, 2, 3, 4 or 5; when n₁ is greater than 1, any two R₁ are the same or different; when n₂ is greater than 1, any two R₂ are the same or different; and when n₃ is greater than 1, any two R₃ are the same or different;
L₁ and L₂ are the same or different, and are each independently selected from a single bond, substituted or unsubstituted arylene with 6 to 30 carbon atoms, and substituted or unsubstituted heteroarylene with 3 to 30 carbon atoms; and
substituents in L₁, L₂, and Ar₁ are the same or different, and are respectively and independently selected from deuterium, cyano, a halogen group, alkyl with 1 to 5 carbon atoms, aryl with 6 to 12 carbon atoms, heteroaryl with 3 to 12 carbon atoms, and trialkylsilyl with 3 to 12 carbon atoms.

3. The organic compound according to claim 1, wherein Ar₁ is selected from substituted or unsubstituted aryl with 6 to 25 carbon atoms, and substituted or unsubstituted heteroaryl with 5 to 20 carbon atoms; or
substituents in Ar₁ are selected from deuterium, cyano, fluorine, alkyl with 1 to 5 carbon atoms, trimethylsilyl, aryl with 6 to 12 carbon atoms, and heteroaryl with 5 to 12 carbon atoms.

4. The organic compound according to claim 1, wherein Ar₁ is selected from substituted or unsubstituted phenyl, substituted or unsubstituted naphthyl, substituted or unsubstituted biphenyl, substituted or unsubstituted diphenylfuranyl, substituted or unsubstituted dibenzothienyl, substituted or unsubstituted fluorenyl, substituted or unsubstituted carbazolyl, substituted or unsubstituted spirofluorenyl, and substituted or unsubstituted phenanthryl; or
substituents in Ar₁ are each independently selected from deuterium, cyano, fluorine, methyl, ethyl, isopropyl, tert-butyl, phenyl, naphthyl, biphenyl, pyridyl and trimethylsilyl.

5. The organic compound according to claim 1, wherein L₁ and L₂ are each independently selected from a single bond, substituted or unsubstituted arylene with 6 to 20 carbon atoms, and substituted or unsubstituted heteroarylene with 10 to 20 carbon atoms; or
substituents in L₁ and L₂ are each independently selected from deuterium, cyano, fluorine, methyl, ethyl, isopropyl, tert-butyl, phenyl, naphthyl, biphenyl, pyridyl and trimethylsilyl.

6. The organic compound according to claim 1, wherein L₁ and L₂ are each independently selected from a single bond, and a substituted or unsubstituted group V, wherein the unsubstituted group V is selected from the group consisting of: 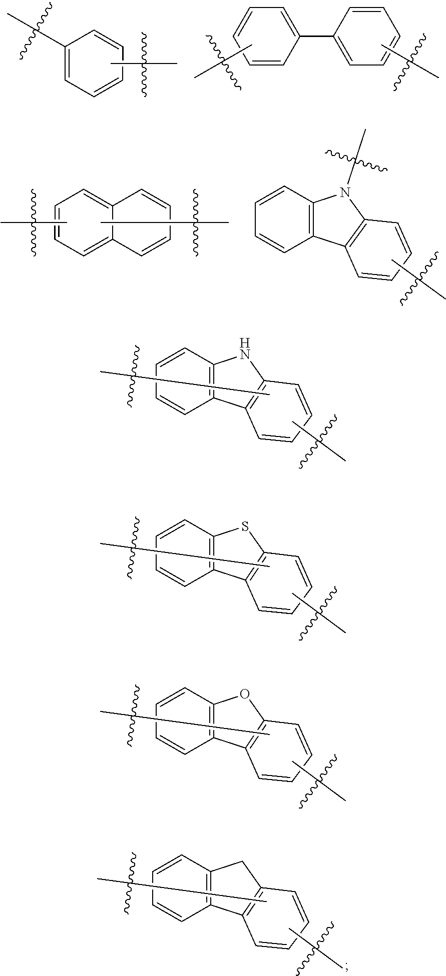
and
the substituted group V has one or two or more substituents, and the substituents are each independently selected from deuterium, cyano, fluorine, methyl, ethyl, isopropyl, tert-butyl, phenyl, naphthyl, biphenyl, pyridyl and trimethylsilyl; and when the number of the substituents is greater than 1, the substituents are the same or different.

7. The organic compound according to claim 1, wherein R₁, R₂ and R₃ are each independently selected from deuterium, cyano, fluorine, methyl, ethyl, isopropyl, tert-butyl, phenyl, naphthyl, biphenyl, pyridyl, trimethylsilyl, dibenzofuranyl and dibenzothienyl.

8. The organic compound according to claim 1, wherein R₄, R₅, R₆, R₇ and R₈ are each independently selected from hydrogen, methyl, ethyl, isopropyl, tert-butyl, phenyl, naphthyl, biphenyl, pyrimidinyl and pyridyl; or, R₄ and R₅ form a fluorene ring; or, R₇ and R₈ form a fluorene ring; or
R₄, R₅, R₆, R₇ and R₈ are each independently selected from methyl, ethyl, isopropyl, tert-butyl, phenyl, naphthyl, biphenyl, pyrimidinyl and pyridyl; or, R₄ and R₅ form a fluorene ring; or, R₇ and R₈ form a fluorene ring.

9. The organic compound according to claim 1, wherein Ar₂ is selected from a substituted or unsubstituted group W, wherein the unsubstituted group W is selected from the group consisting of: 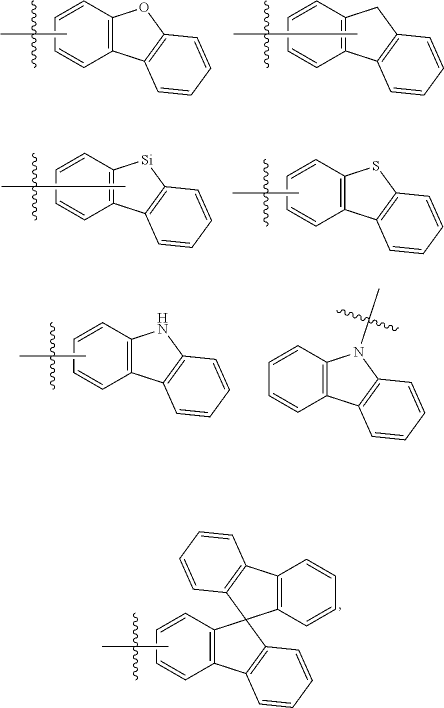
and
the substituted group W has one or two or more substituents, and the substituents are each independently selected from deuterium, cyano, fluorine, methyl, ethyl, isopropyl, tert-butyl, phenyl, naphthyl, biphenyl, pyridyl and trimethylsilyl; and when the number of the substituents is greater than 1, the substituents are the same or different.

10. The organic compound according to claim 1, wherein Ar₂ is selected from the group consisting of:

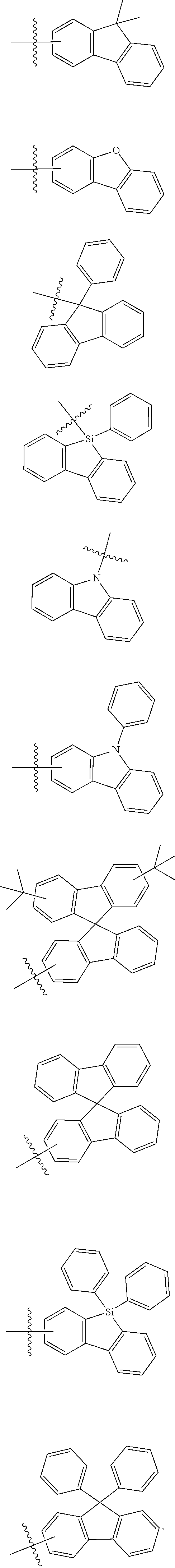

11. The organic compound according to claim 1, wherein A is selected from the group consisting of:

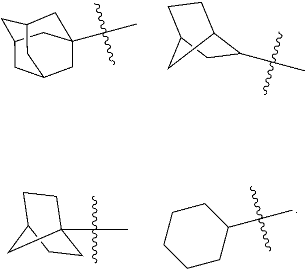 [SMILES: *C12CC3CC(CC(C3)C1)C2 ; *C1CC2CCC1C2 ; *C12CCC(CC1)C2 ; *C1CCCCC1]

12. The organic compound according to claim 1, wherein the organic compound is selected from the following compounds:

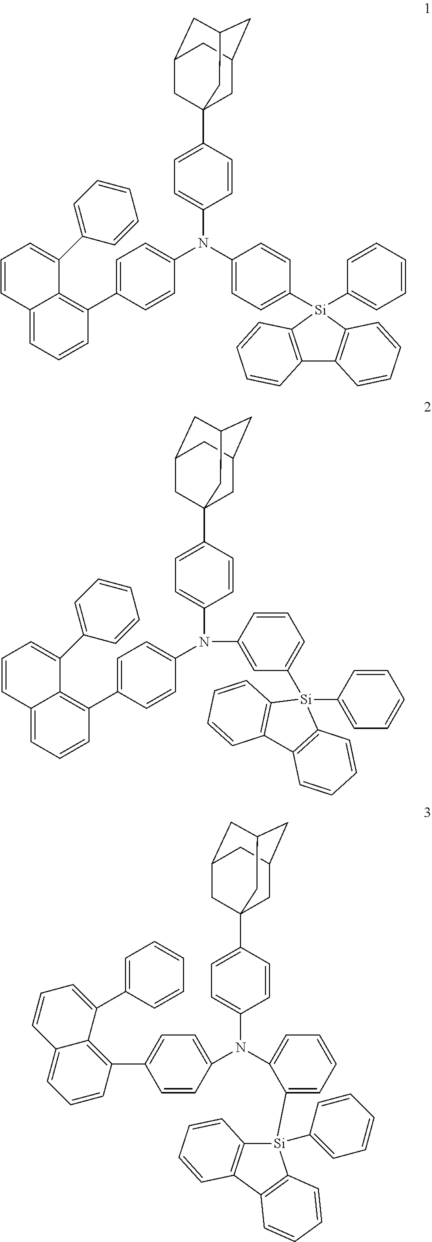

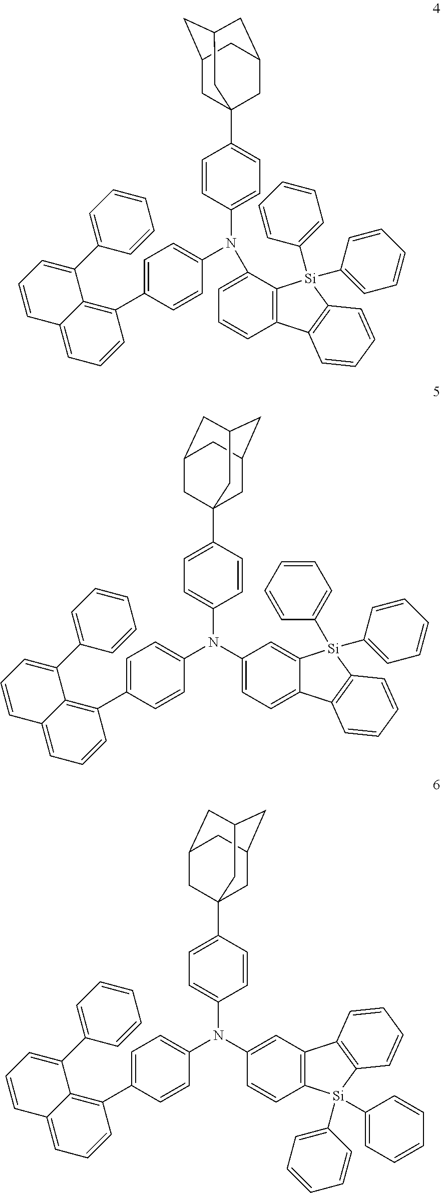 [SMILES: c1ccc(-c2cccc3cccc(-c4ccc(N(c5ccc(C67CC8CC(CC(C8)C6)C7)cc5)c5cccc6c5[Si](c5ccccc5)(c5ccccc5)c5ccccc5-6)cc4)c23)cc1 ; c1ccc(-c2cccc3cccc(-c4ccc(N(c5ccc(C67CC8CC(CC(C8)C6)C7)cc5)c5ccc6c(c5)[Si](c5ccccc5)(c5ccccc5)c5ccccc5-6)cc4)c23)cc1 ; c1ccc(-c2cccc3cccc(-c4ccc(N(c5ccc(C67CC8CC(CC(C8)C6)C7)cc5)c5ccc6c(c5)-c5ccccc5[Si]6(c5ccccc5)c5ccccc5)cc4)c23)cc1]

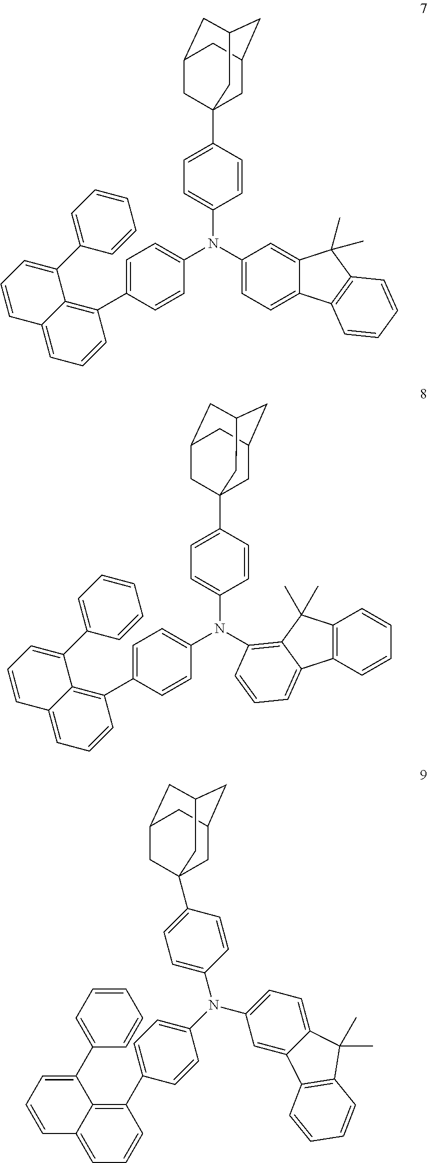 [SMILES: CC1(C)c2ccccc2-c2ccc(N(c3ccc(-c4cccc5cccc(-c6ccccc6)c45)cc3)c3ccc(C45CC6CC(CC(C6)C4)C5)cc3)cc21 ; CC1(C)c2ccccc2-c2cccc(N(c3ccc(-c4cccc5cccc(-c6ccccc6)c45)cc3)c3ccc(C45CC6CC(CC(C6)C4)C5)cc3)c21 ; CC1(C)c2ccccc2-c2cc(N(c3ccc(-c4cccc5cccc(-c6ccccc6)c45)cc3)c3ccc(C45CC6CC(CC(C6)C4)C5)cc3)ccc21]

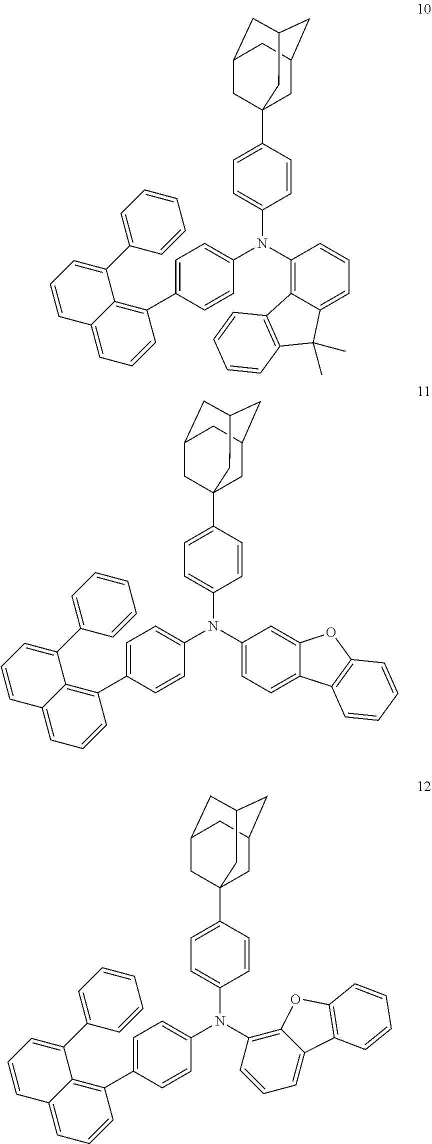

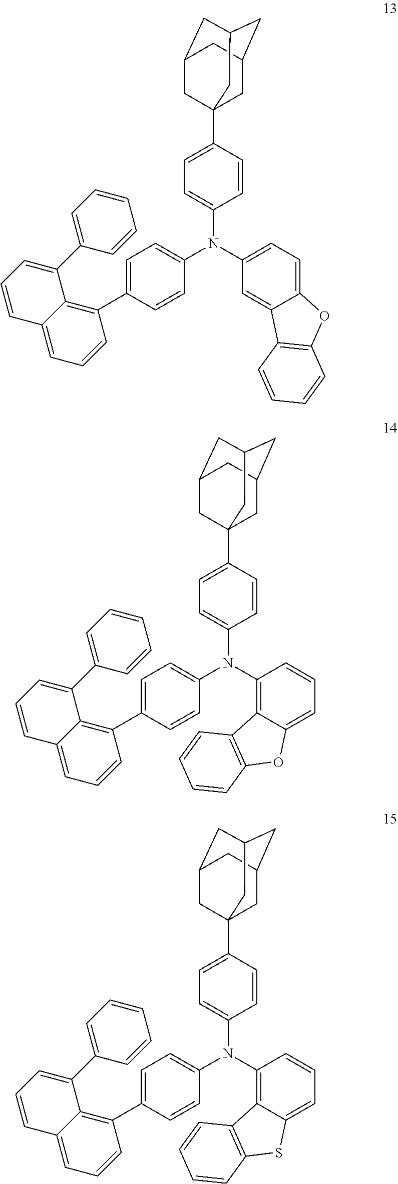

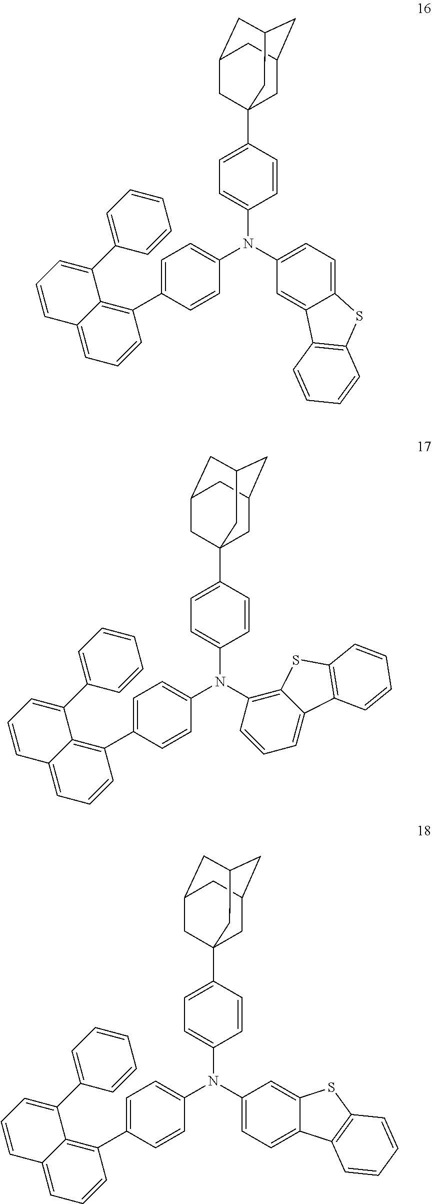 [SMILES: c1ccc(-c2cccc3cccc(-c4ccc(N(c5ccc(C67CC8CC(CC(C8)C6)C7)cc5)c5ccc6sc7ccccc7c6c5)cc4)c23)cc1 ; c1ccc(-c2cccc3cccc(-c4ccc(N(c5ccc(C67CC8CC(CC(C8)C6)C7)cc5)c5cccc6c5sc5ccccc56)cc4)c23)cc1 ; c1ccc(-c2cccc3cccc(-c4ccc(N(c5ccc(C67CC8CC(CC(C8)C6)C7)cc5)c5ccc6c(c5)sc5ccccc56)cc4)c23)cc1]

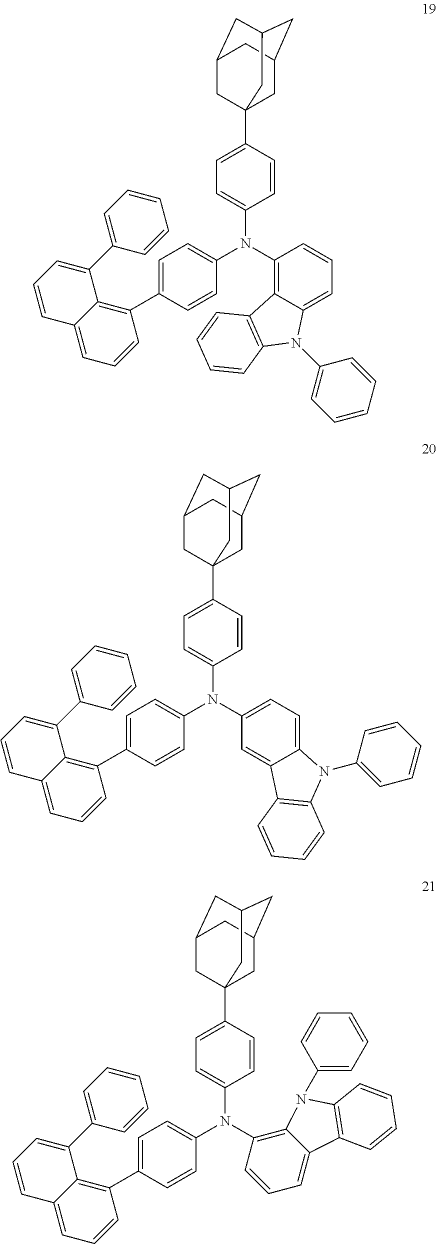

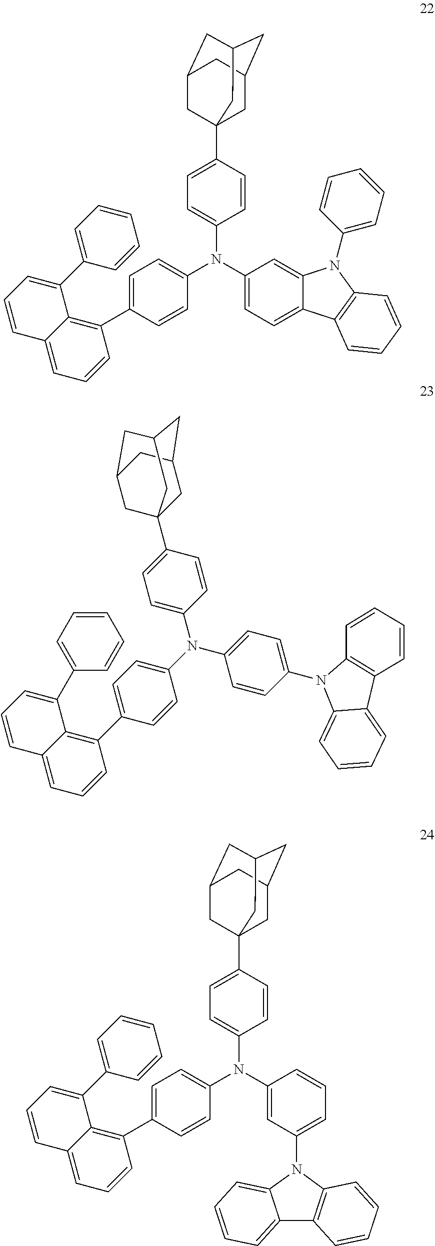 [SMILES: c1ccc(-c2cccc3cccc(-c4ccc(N(c5ccc(C67CC8CC(CC(C8)C6)C7)cc5)c5ccc6c7ccccc7n(-c7ccccc7)c6c5)cc4)c23)cc1 ; c1ccc(-c2cccc3cccc(-c4ccc(N(c5ccc(-n6c7ccccc7c7ccccc76)cc5)c5ccc(C67CC8CC(CC(C8)C6)C7)cc5)cc4)c23)cc1 ; c1ccc(-c2cccc3cccc(-c4ccc(N(c5ccc(C67CC8CC(CC(C8)C6)C7)cc5)c5cccc(-n6c7ccccc7c7ccccc76)c5)cc4)c23)cc1]

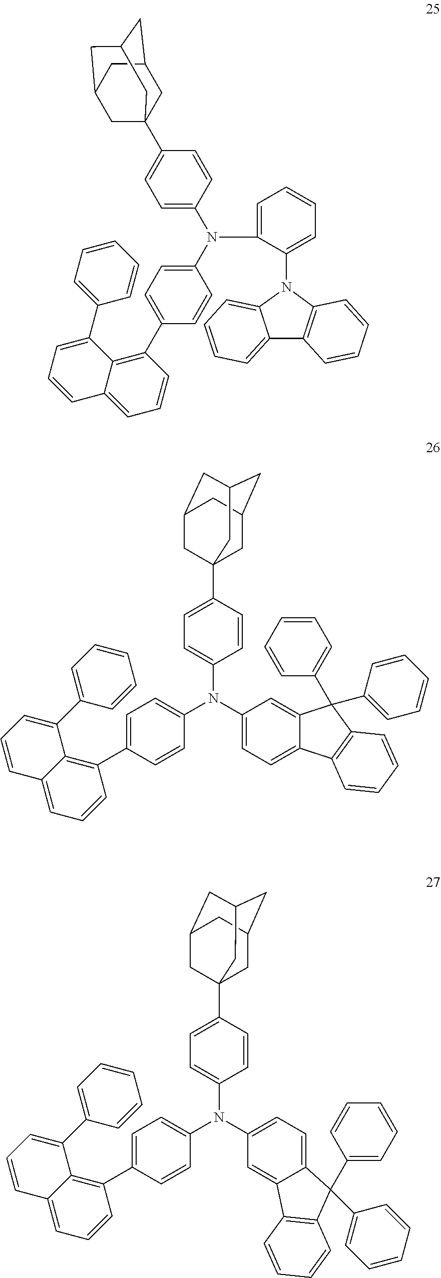 [SMILES: c1ccc(-c2cccc3cccc(-c4ccc(N(c5ccc(C67CC8CC(CC(C8)C6)C7)cc5)c5ccccc5-n5c6ccccc6c6ccccc65)cc4)c23)cc1 ; c1ccc(-c2cccc3cccc(-c4ccc(N(c5ccc(C67CC8CC(CC(C8)C6)C7)cc5)c5ccc6c(c5)C(c5ccccc5)(c5ccccc5)c5ccccc5-6)cc4)c23)cc1 ; c1ccc(-c2cccc3cccc(-c4ccc(N(c5ccc(C67CC8CC(CC(C8)C6)C7)cc5)c5ccc6c(c5)-c5ccccc5C6(c5ccccc5)c5ccccc5)cc4)c23)cc1]

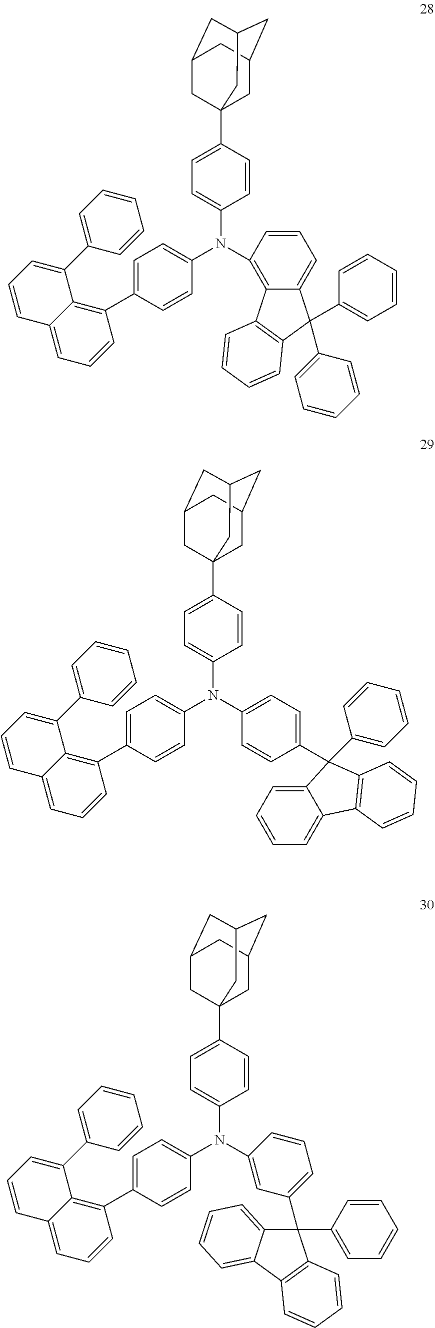 [SMILES: c1ccc(-c2cccc3cccc(-c4ccc(N(c5ccc(C67CC8CC(CC(C8)C6)C7)cc5)c5cccc6c5-c5ccccc5C6(c5ccccc5)c5ccccc5)cc4)c23)cc1 ; c1ccc(-c2cccc3cccc(-c4ccc(N(c5ccc(C67CC8CC(CC(C8)C6)C7)cc5)c5ccc(C6(c7ccccc7)c7ccccc7-c7ccccc76)cc5)cc4)c23)cc1 ; c1ccc(-c2cccc3cccc(-c4ccc(N(c5ccc(C67CC8CC(CC(C8)C6)C7)cc5)c5cccc(C6(c7ccccc7)c7ccccc7-c7ccccc76)c5)cc4)c23)cc1]

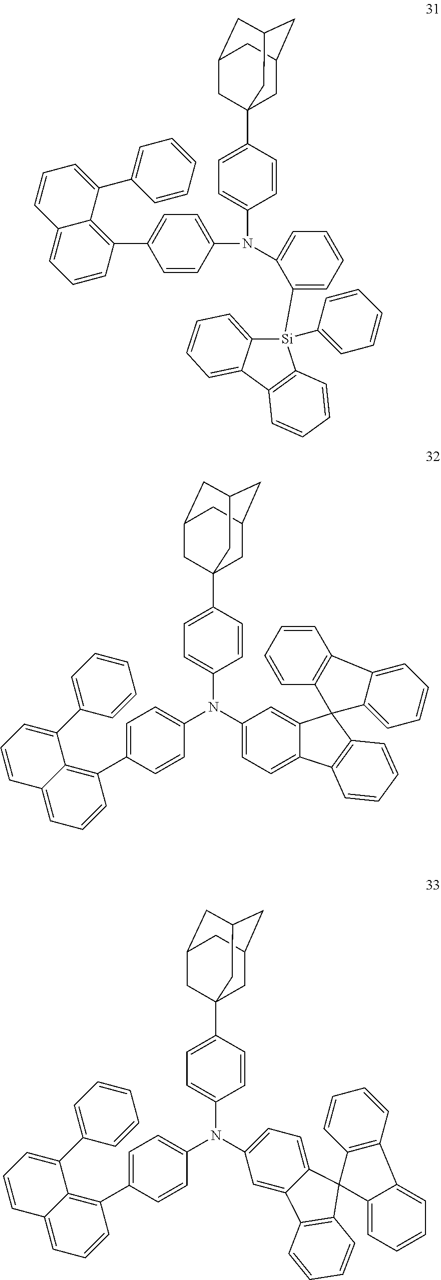 [SMILES: c1ccc(-c2cccc3cccc(-c4ccc(N(c5ccc(C67CC8CC(CC(C8)C6)C7)cc5)c5ccccc5[Si]5(c6ccccc6)c6ccccc6-c6ccccc65)cc4)c23)cc1 ; c1ccc(-c2cccc3cccc(-c4ccc(N(c5ccc(C67CC8CC(CC(C8)C6)C7)cc5)c5ccc6c(c5)C5(c7ccccc7-c7ccccc75)c5ccccc5-6)cc4)c23)cc1 ; c1ccc(-c2cccc3cccc(-c4ccc(N(c5ccc(C67CC8CC(CC(C8)C6)C7)cc5)c5ccc6c(c5)-c5ccccc5C65c6ccccc6-c6ccccc65)cc4)c23)cc1]

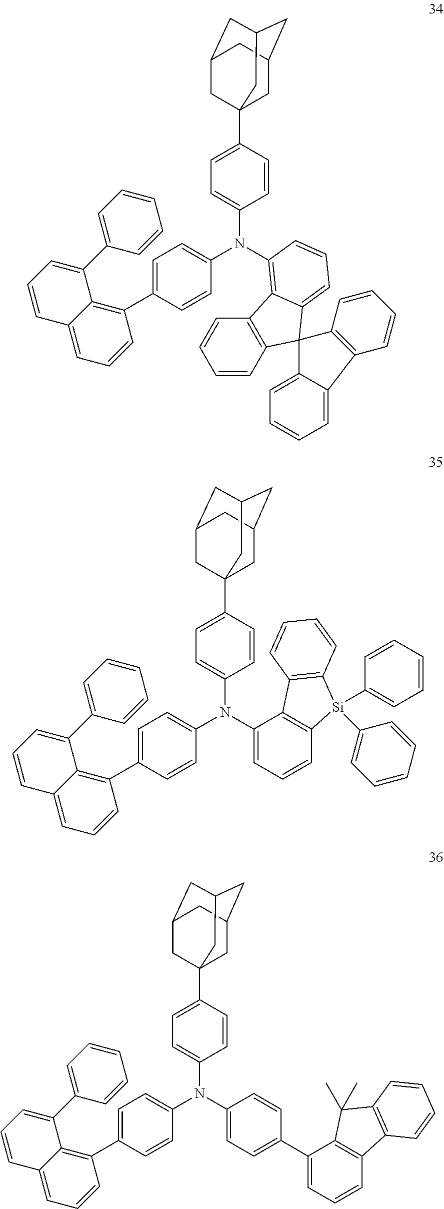 [SMILES: c1ccc(-c2cccc3cccc(-c4ccc(N(c5ccc(C67CC8CC(CC(C8)C6)C7)cc5)c5cccc6c5-c5ccccc5C65c6ccccc6-c6ccccc65)cc4)c23)cc1 ; c1ccc(-c2cccc3cccc(-c4ccc(N(c5ccc(C67CC8CC(CC(C8)C6)C7)cc5)c5cccc6c5-c5ccccc5[Si]6(c5ccccc5)c5ccccc5)cc4)c23)cc1 ; CC1(C)c2ccccc2-c2cccc(-c3ccc(N(c4ccc(-c5cccc6cccc(-c7ccccc7)c56)cc4)c4ccc(C56CC7CC(CC(C7)C5)C6)cc4)cc3)c21]

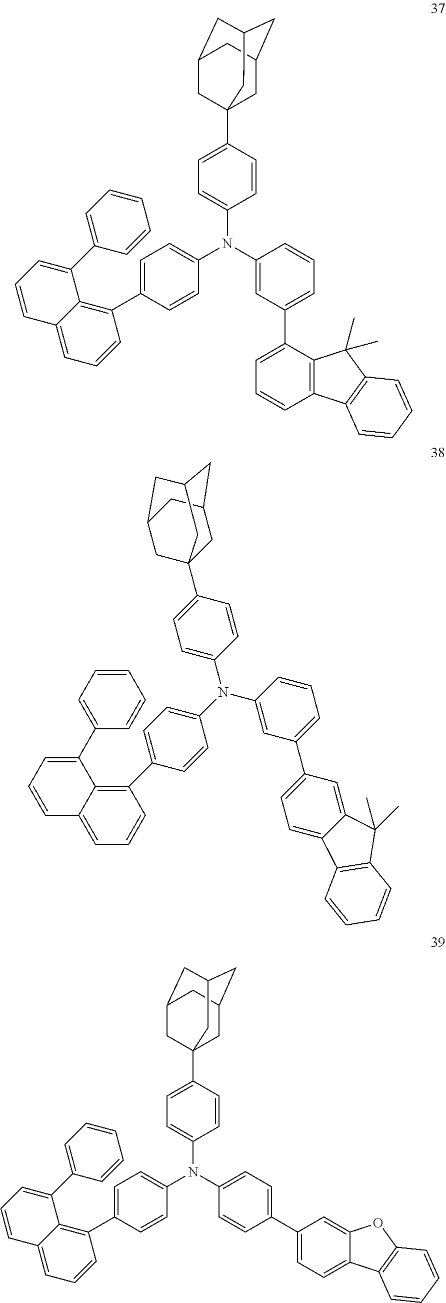 [SMILES: CC1(C)c2ccccc2-c2cccc(-c3cccc(N(c4ccc(-c5cccc6cccc(-c7ccccc7)c56)cc4)c4ccc(C56CC7CC(CC(C7)C5)C6)cc4)c3)c21 ; CC1(C)c2ccccc2-c2ccc(-c3cccc(N(c4ccc(-c5cccc6cccc(-c7ccccc7)c56)cc4)c4ccc(C56CC7CC(CC(C7)C5)C6)cc4)c3)cc21 ; c1ccc(-c2cccc3cccc(-c4ccc(N(c5ccc(-c6ccc7c(c6)oc6ccccc67)cc5)c5ccc(C67CC8CC(CC(C8)C6)C7)cc5)cc4)c23)cc1]

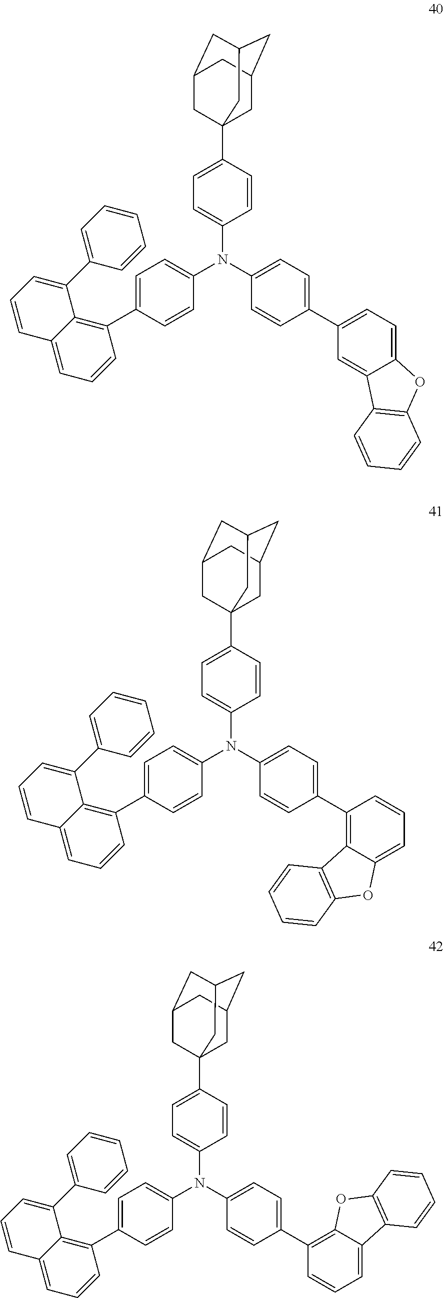 [SMILES: c1ccc(-c2cccc3cccc(-c4ccc(N(c5ccc(-c6ccc7oc8ccccc8c7c6)cc5)c5ccc(C67CC8CC(CC(C8)C6)C7)cc5)cc4)c23)cc1 ; c1ccc(-c2cccc3cccc(-c4ccc(N(c5ccc(-c6cccc7oc8ccccc8c67)cc5)c5ccc(C67CC8CC(CC(C8)C6)C7)cc5)cc4)c23)cc1 ; c1ccc(-c2cccc3cccc(-c4ccc(N(c5ccc(-c6cccc7c6oc6ccccc67)cc5)c5ccc(C67CC8CC(CC(C8)C6)C7)cc5)cc4)c23)cc1]

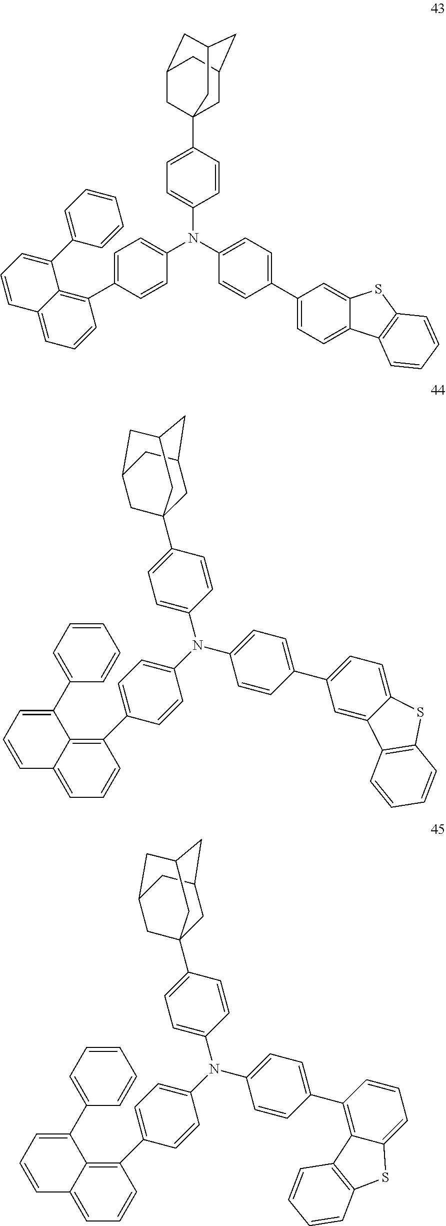 [SMILES: c1ccc(-c2cccc3cccc(-c4ccc(N(c5ccc(-c6ccc7c(c6)sc6ccccc67)cc5)c5ccc(C67CC8CC(CC(C8)C6)C7)cc5)cc4)c23)cc1 ; c1ccc(-c2cccc3cccc(-c4ccc(N(c5ccc(-c6ccc7sc8ccccc8c7c6)cc5)c5ccc(C67CC8CC(CC(C8)C6)C7)cc5)cc4)c23)cc1 ; c1ccc(-c2cccc3cccc(-c4ccc(N(c5ccc(-c6cccc7sc8ccccc8c67)cc5)c5ccc(C67CC8CC(CC(C8)C6)C7)cc5)cc4)c23)cc1]

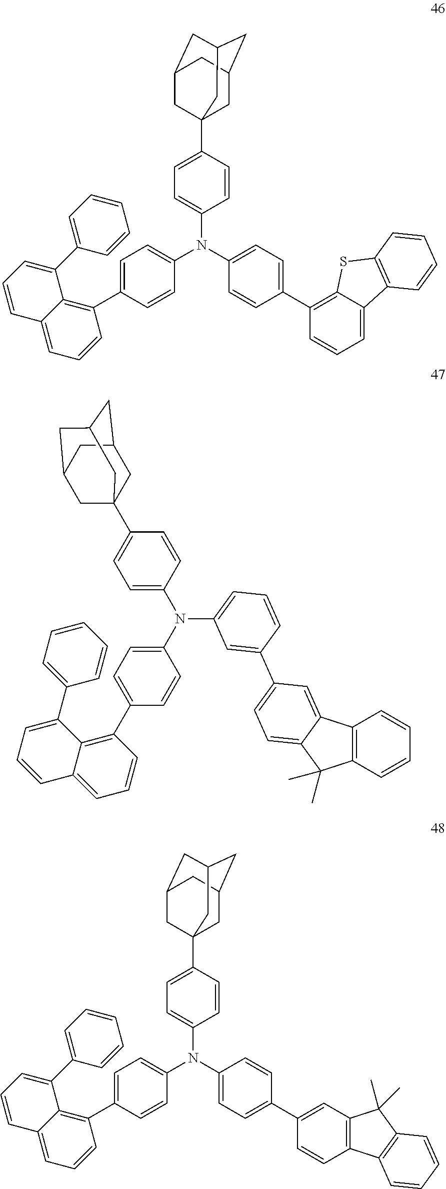 [SMILES: c1ccc(-c2cccc3cccc(-c4ccc(N(c5ccc(-c6cccc7c6sc6ccccc67)cc5)c5ccc(C67CC8CC(CC(C8)C6)C7)cc5)cc4)c23)cc1 ; CC1(C)c2ccccc2-c2cc(-c3cccc(N(c4ccc(-c5cccc6cccc(-c7ccccc7)c56)cc4)c4ccc(C56CC7CC(CC(C7)C5)C6)cc4)c3)ccc21 ; CC1(C)c2ccccc2-c2ccc(-c3ccc(N(c4ccc(-c5cccc6cccc(-c7ccccc7)c56)cc4)c4ccc(C56CC7CC(CC(C7)C5)C6)cc4)cc3)cc21]

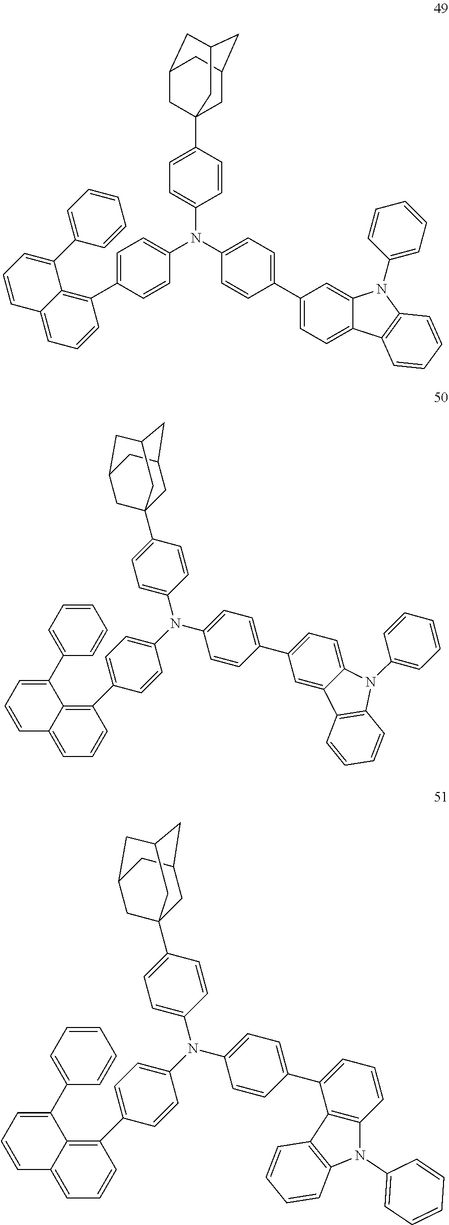 [SMILES: c1ccc(-c2cccc3cccc(-c4ccc(N(c5ccc(-c6ccc7c8ccccc8n(-c8ccccc8)c7c6)cc5)c5ccc(C67CC8CC(CC(C8)C6)C7)cc5)cc4)c23)cc1 ; c1ccc(-c2cccc3cccc(-c4ccc(N(c5ccc(-c6ccc7c(c6)c6ccccc6n7-c6ccccc6)cc5)c5ccc(C67CC8CC(CC(C8)C6)C7)cc5)cc4)c23)cc1 ; c1ccc(-c2cccc3cccc(-c4ccc(N(c5ccc(-c6cccc7c6c6ccccc6n7-c6ccccc6)cc5)c5ccc(C67CC8CC(CC(C8)C6)C7)cc5)cc4)c23)cc1]

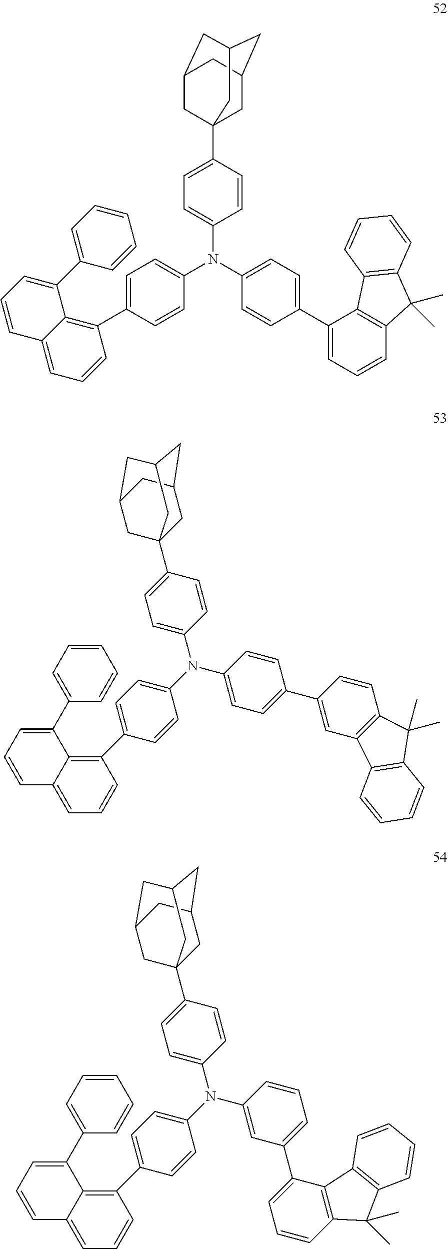 [SMILES: CC1(C)c2ccccc2-c2c(-c3ccc(N(c4ccc(-c5cccc6cccc(-c7ccccc7)c56)cc4)c4ccc(C56CC7CC(CC(C7)C5)C6)cc4)cc3)cccc21 ; CC1(C)c2ccccc2-c2cc(-c3ccc(N(c4ccc(-c5cccc6cccc(-c7ccccc7)c56)cc4)c4ccc(C56CC7CC(CC(C7)C5)C6)cc4)cc3)ccc21 ; CC1(C)c2ccccc2-c2c(-c3cccc(N(c4ccc(-c5cccc6cccc(-c7ccccc7)c56)cc4)c4ccc(C56CC7CC(CC(C7)C5)C6)cc4)c3)cccc21]

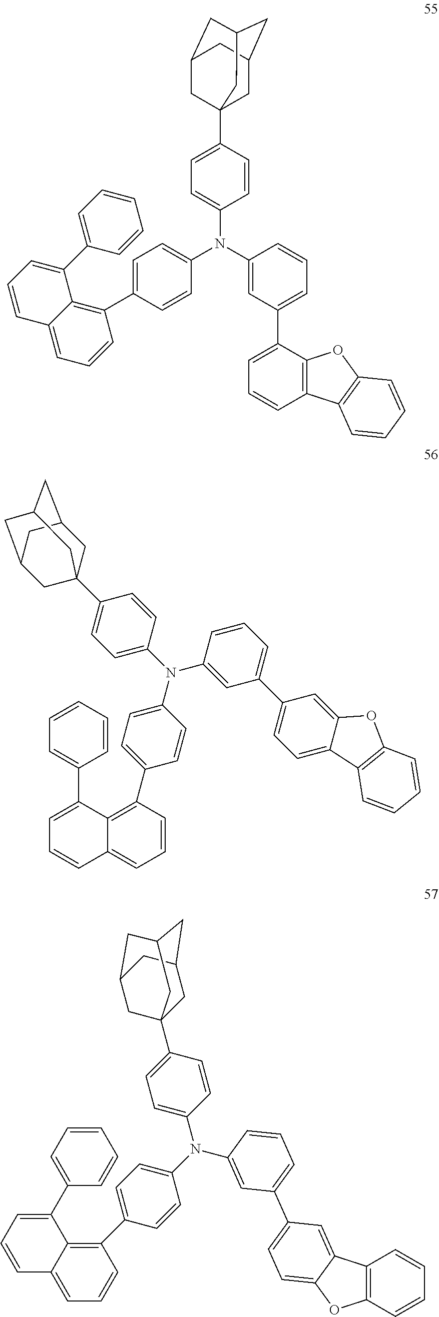 [SMILES: c1ccc(-c2cccc3cccc(-c4ccc(N(c5ccc(C67CC8CC(CC(C8)C6)C7)cc5)c5cccc(-c6cccc7c6oc6ccccc67)c5)cc4)c23)cc1 ; c1ccc(-c2cccc3cccc(-c4ccc(N(c5ccc(C67CC8CC(CC(C8)C6)C7)cc5)c5cccc(-c6ccc7c(c6)oc6ccccc67)c5)cc4)c23)cc1 ; c1ccc(-c2cccc3cccc(-c4ccc(N(c5ccc(C67CC8CC(CC(C8)C6)C7)cc5)c5cccc(-c6ccc7oc8ccccc8c7c6)c5)cc4)c23)cc1]

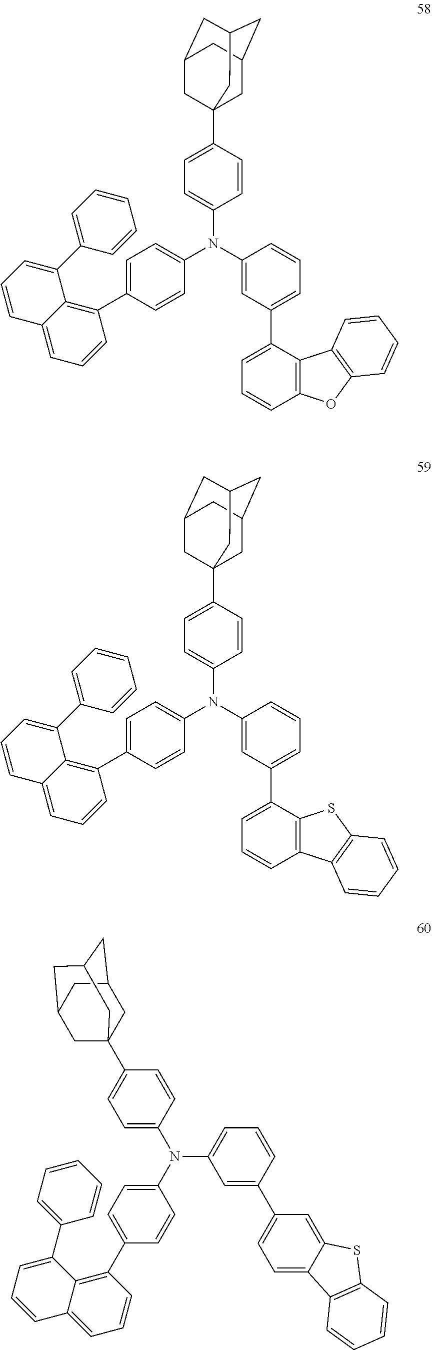 [SMILES: c1ccc(-c2cccc3cccc(-c4ccc(N(c5ccc(C67CC8CC(CC(C8)C6)C7)cc5)c5cccc(-c6cccc7oc8ccccc8c67)c5)cc4)c23)cc1 ; c1ccc(-c2cccc3cccc(-c4ccc(N(c5ccc(C67CC8CC(CC(C8)C6)C7)cc5)c5cccc(-c6cccc7c6sc6ccccc67)c5)cc4)c23)cc1 ; c1ccc(-c2cccc3cccc(-c4ccc(N(c5ccc(C67CC8CC(CC(C8)C6)C7)cc5)c5cccc(-c6ccc7c(c6)sc6ccccc67)c5)cc4)c23)cc1]

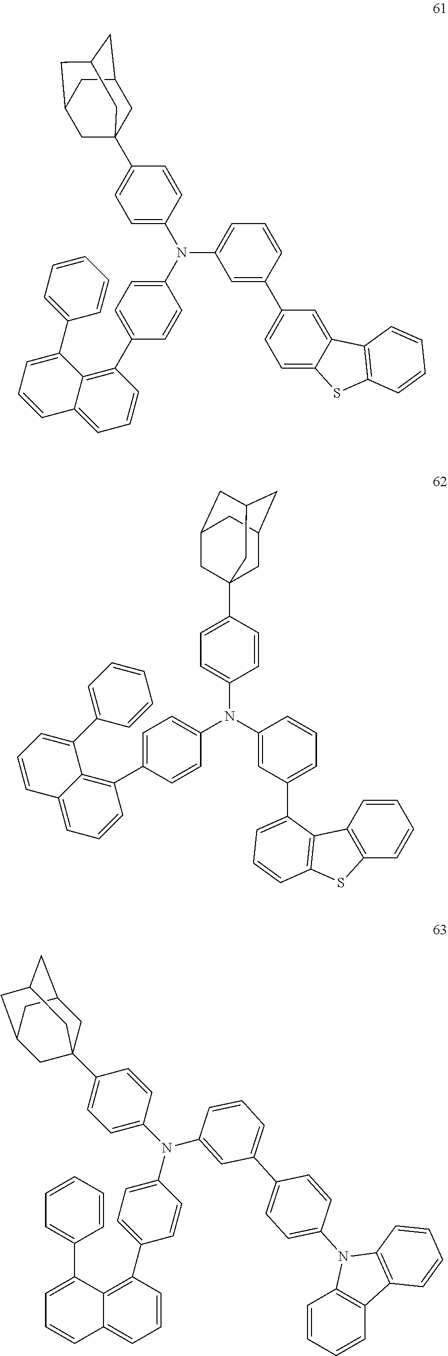 [SMILES: c1ccc(-c2cccc3cccc(-c4ccc(N(c5ccc(C67CC8CC(CC(C8)C6)C7)cc5)c5cccc(-c6ccc7sc8ccccc8c7c6)c5)cc4)c23)cc1 ; c1ccc(-c2cccc3cccc(-c4ccc(N(c5ccc(C67CC8CC(CC(C8)C6)C7)cc5)c5cccc(-c6cccc7sc8ccccc8c67)c5)cc4)c23)cc1 ; c1ccc(-c2cccc3cccc(-c4ccc(N(c5ccc(C67CC8CC(CC(C8)C6)C7)cc5)c5cccc(-c6ccc(-n7c8ccccc8c8ccccc87)cc6)c5)cc4)c23)cc1]

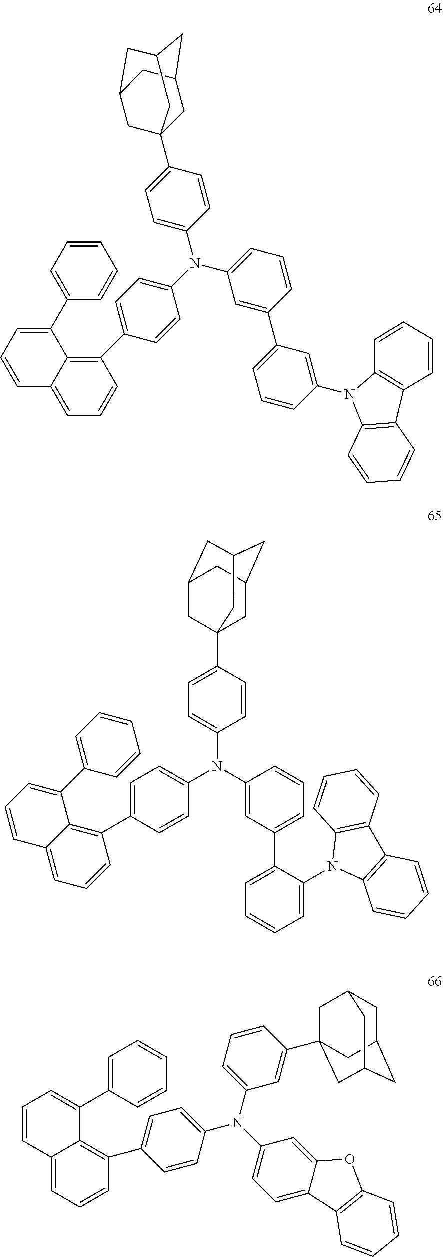 [SMILES: c1ccc(-c2cccc3cccc(-c4ccc(N(c5ccc(C67CC8CC(CC(C8)C6)C7)cc5)c5cccc(-c6cccc(-n7c8ccccc8c8ccccc87)c6)c5)cc4)c23)cc1 ; c1ccc(-c2cccc3cccc(-c4ccc(N(c5ccc(C67CC8CC(CC(C8)C6)C7)cc5)c5cccc(-c6ccccc6-n6c7ccccc7c7ccccc76)c5)cc4)c23)cc1 ; c1ccc(-c2cccc3cccc(-c4ccc(N(c5cccc(C67CC8CC(CC(C8)C6)C7)c5)c5ccc6c(c5)oc5ccccc56)cc4)c23)cc1]

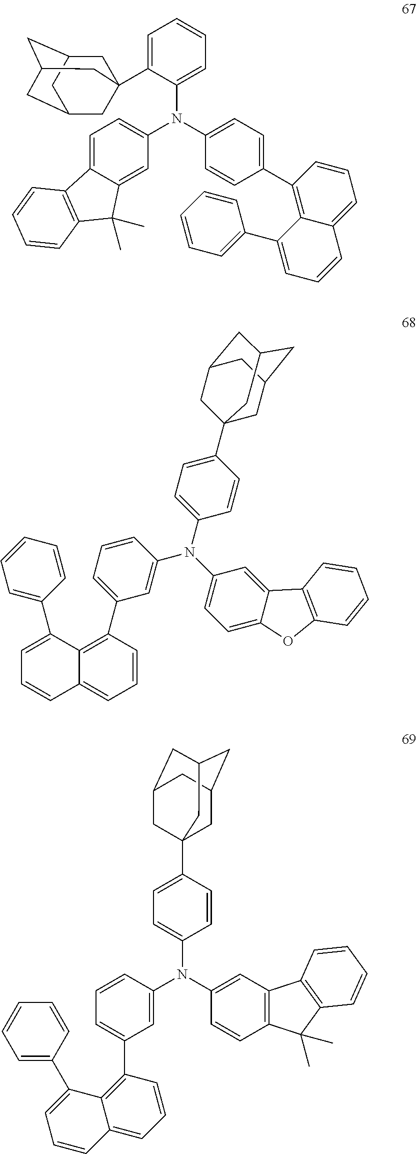 [SMILES: CC1(C)c2ccccc2-c2ccc(N(c3ccc(-c4cccc5cccc(-c6ccccc6)c45)cc3)c3ccccc3C34CC5CC(CC(C5)C3)C4)cc21 ; c1ccc(-c2cccc3cccc(-c4cccc(N(c5ccc(C67CC8CC(CC(C8)C6)C7)cc5)c5ccc6oc7ccccc7c6c5)c4)c23)cc1 ; CC1(C)c2ccccc2-c2cc(N(c3ccc(C45CC6CC(CC(C6)C4)C5)cc3)c3cccc(-c4cccc5cccc(-c6ccccc6)c45)c3)ccc21]

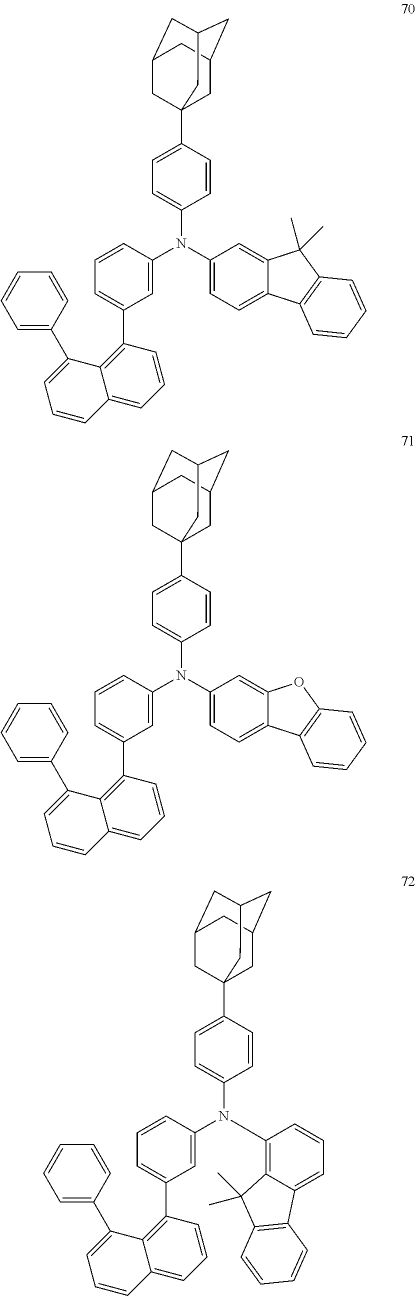 [SMILES: CC1(C)c2ccccc2-c2ccc(N(c3ccc(C45CC6CC(CC(C6)C4)C5)cc3)c3cccc(-c4cccc5cccc(-c6ccccc6)c45)c3)cc21 ; c1ccc(-c2cccc3cccc(-c4cccc(N(c5ccc(C67CC8CC(CC(C8)C6)C7)cc5)c5ccc6c(c5)oc5ccccc56)c4)c23)cc1 ; CC1(C)c2ccccc2-c2cccc(N(c3ccc(C45CC6CC(CC(C6)C4)C5)cc3)c3cccc(-c4cccc5cccc(-c6ccccc6)c45)c3)c21]

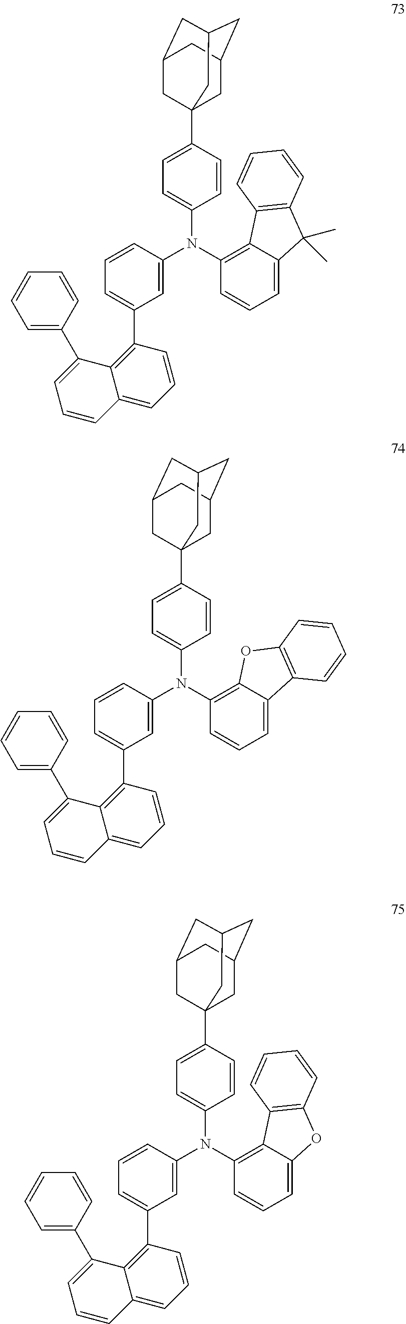 [SMILES: CC1(C)c2ccccc2-c2c(N(c3ccc(C45CC6CC(CC(C6)C4)C5)cc3)c3cccc(-c4cccc5cccc(-c6ccccc6)c45)c3)cccc21 ; c1ccc(-c2cccc3cccc(-c4cccc(N(c5ccc(C67CC8CC(CC(C8)C6)C7)cc5)c5cccc6c5oc5ccccc56)c4)c23)cc1 ; c1ccc(-c2cccc3cccc(-c4cccc(N(c5ccc(C67CC8CC(CC(C8)C6)C7)cc5)c5cccc6oc7ccccc7c56)c4)c23)cc1]

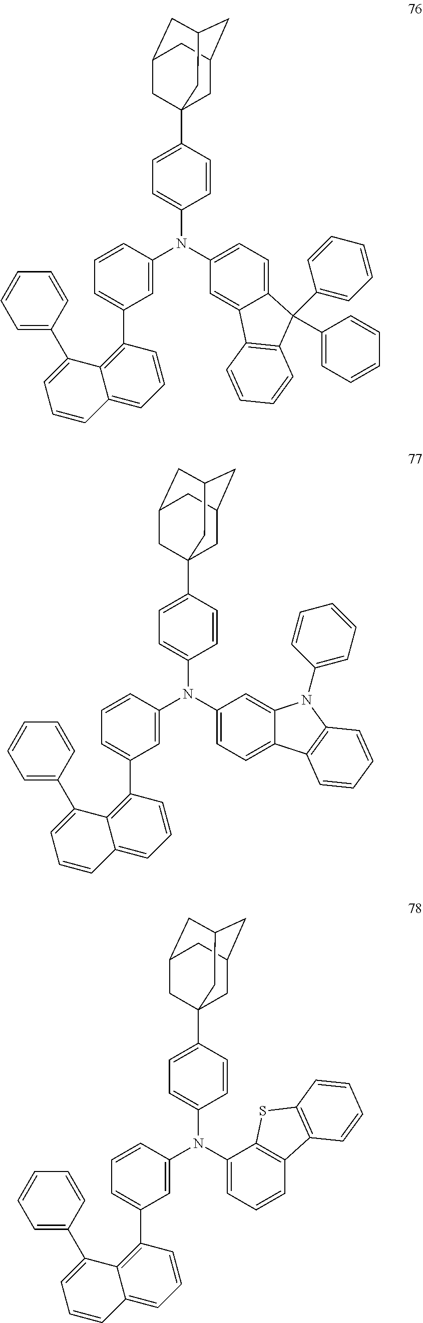 [SMILES: c1ccc(-c2cccc3cccc(-c4cccc(N(c5ccc(C67CC8CC(CC(C8)C6)C7)cc5)c5ccc6c(c5)-c5ccccc5C6(c5ccccc5)c5ccccc5)c4)c23)cc1 ; c1ccc(-c2cccc3cccc(-c4cccc(N(c5ccc(C67CC8CC(CC(C8)C6)C7)cc5)c5ccc6c7ccccc7n(-c7ccccc7)c6c5)c4)c23)cc1 ; c1ccc(-c2cccc3cccc(-c4cccc(N(c5ccc(C67CC8CC(CC(C8)C6)C7)cc5)c5cccc6c5sc5ccccc56)c4)c23)cc1]

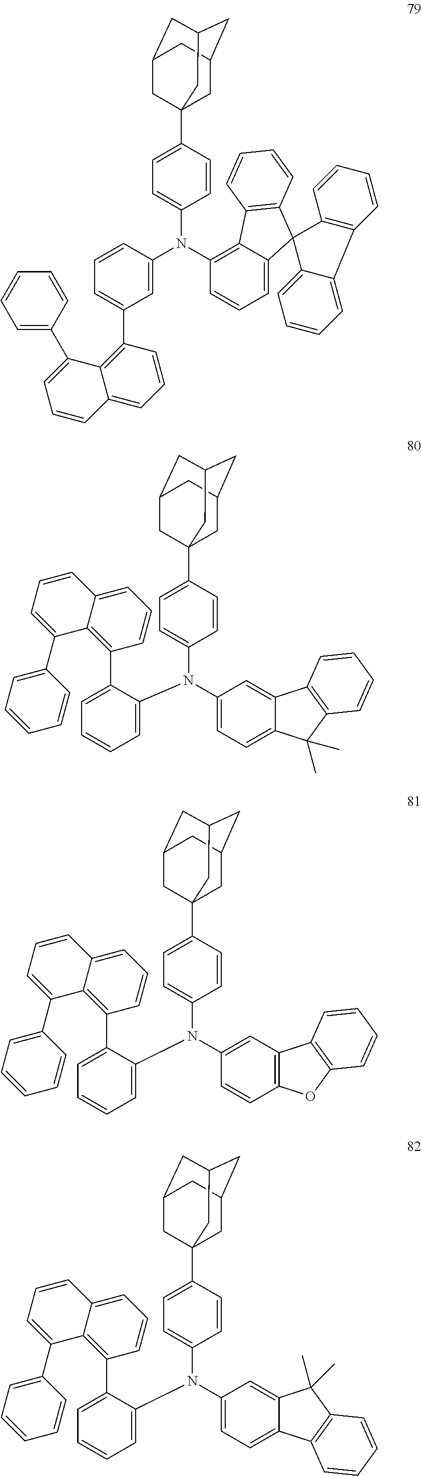 [SMILES: c1ccc(-c2cccc3cccc(-c4cccc(N(c5ccc(C67CC8CC(CC(C8)C6)C7)cc5)c5cccc6c5-c5ccccc5C65c6ccccc6-c6ccccc65)c4)c23)cc1 ; CC1(C)c2ccccc2-c2cc(N(c3ccc(C45CC6CC(CC(C6)C4)C5)cc3)c3ccccc3-c3cccc4cccc(-c5ccccc5)c34)ccc21 ; c1ccc(-c2cccc3cccc(-c4ccccc4N(c4ccc(C56CC7CC(CC(C7)C5)C6)cc4)c4ccc5oc6ccccc6c5c4)c23)cc1 ; CC1(C)c2ccccc2-c2ccc(N(c3ccc(C45CC6CC(CC(C6)C4)C5)cc3)c3ccccc3-c3cccc4cccc(-c5ccccc5)c34)cc21]

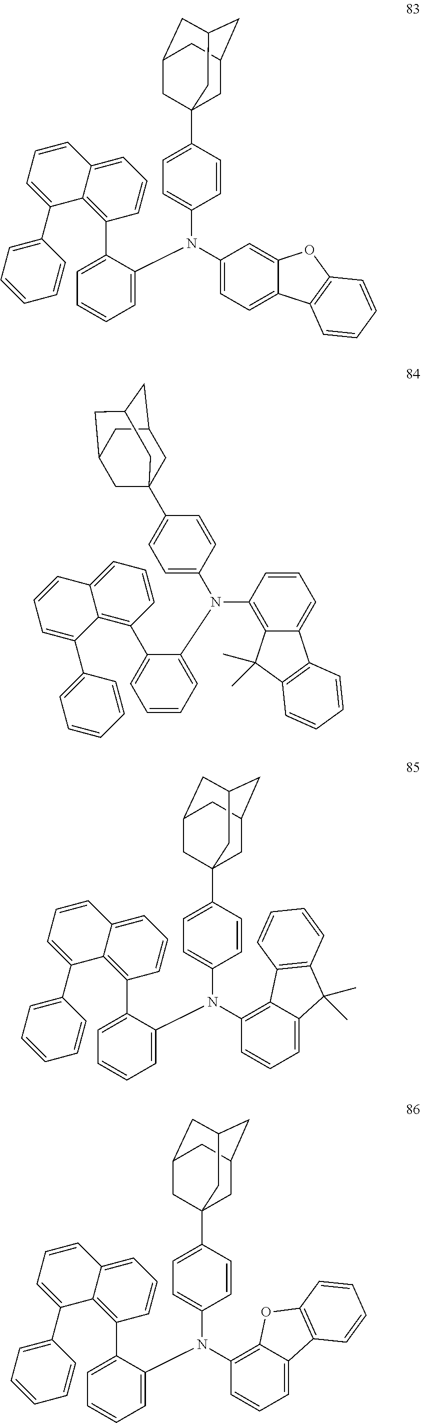 [SMILES: c1ccc(-c2cccc3cccc(-c4ccccc4N(c4ccc(C56CC7CC(CC(C7)C5)C6)cc4)c4ccc5c(c4)oc4ccccc45)c23)cc1 ; CC1(C)c2ccccc2-c2cccc(N(c3ccc(C45CC6CC(CC(C6)C4)C5)cc3)c3ccccc3-c3cccc4cccc(-c5ccccc5)c34)c21 ; CC1(C)c2ccccc2-c2c(N(c3ccc(C45CC6CC(CC(C6)C4)C5)cc3)c3ccccc3-c3cccc4cccc(-c5ccccc5)c34)cccc21 ; c1ccc(-c2cccc3cccc(-c4ccccc4N(c4ccc(C56CC7CC(CC(C7)C5)C6)cc4)c4cccc5c4oc4ccccc45)c23)cc1]

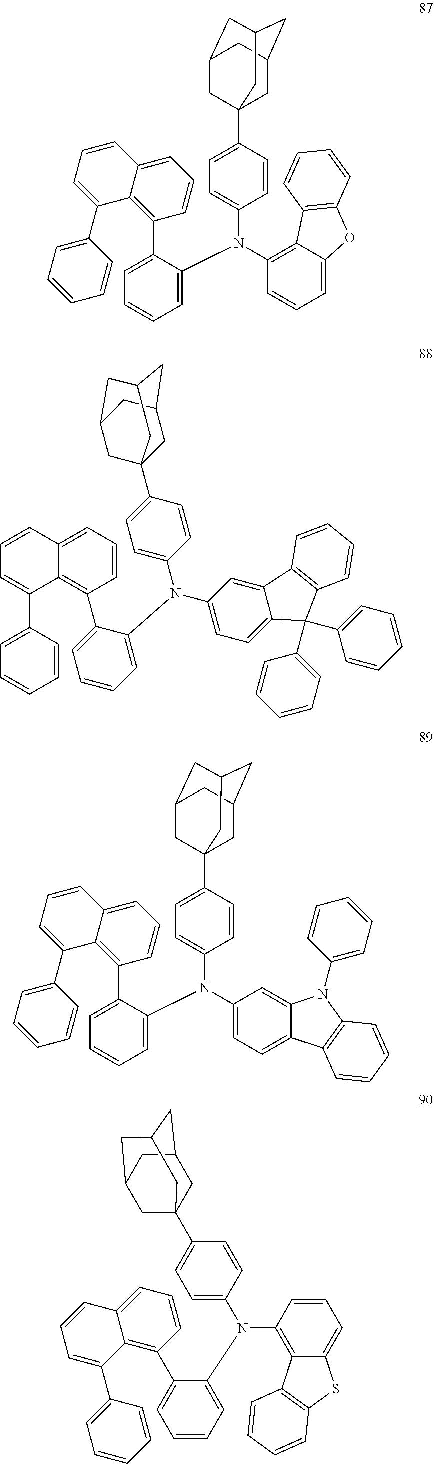

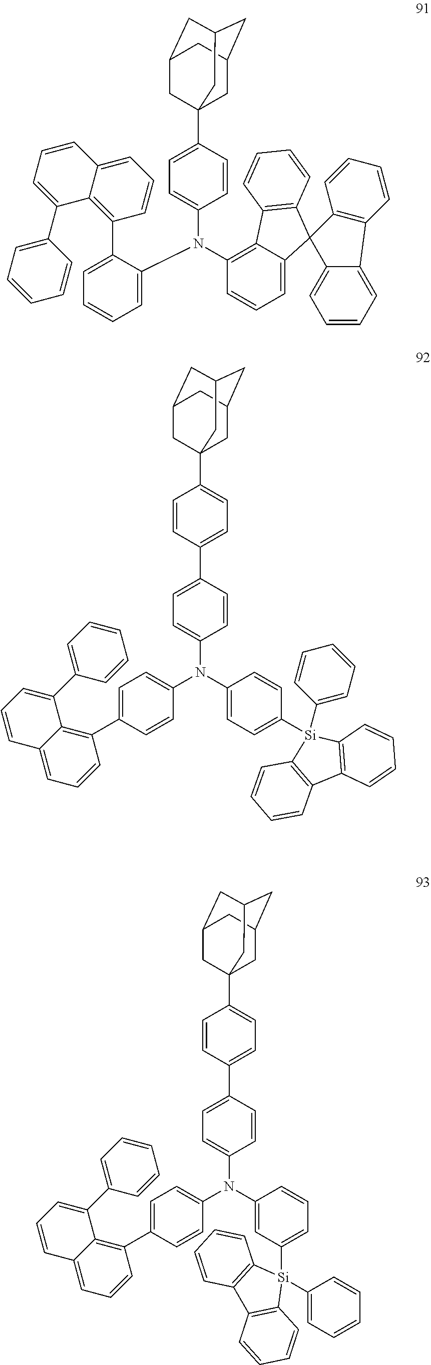 [SMILES: c1ccc(-c2cccc3cccc(-c4ccccc4N(c4ccc(C56CC7CC(CC(C7)C5)C6)cc4)c4cccc5c4-c4ccccc4C54c5ccccc5-c5ccccc54)c23)cc1 ; c1ccc(-c2cccc3cccc(-c4ccc(N(c5ccc(-c6ccc(C78CC9CC(CC(C9)C7)C8)cc6)cc5)c5ccc([Si]6(c7ccccc7)c7ccccc7-c7ccccc76)cc5)cc4)c23)cc1 ; c1ccc(-c2cccc3cccc(-c4ccc(N(c5ccc(-c6ccc(C78CC9CC(CC(C9)C7)C8)cc6)cc5)c5cccc([Si]6(c7ccccc7)c7ccccc7-c7ccccc76)c5)cc4)c23)cc1]

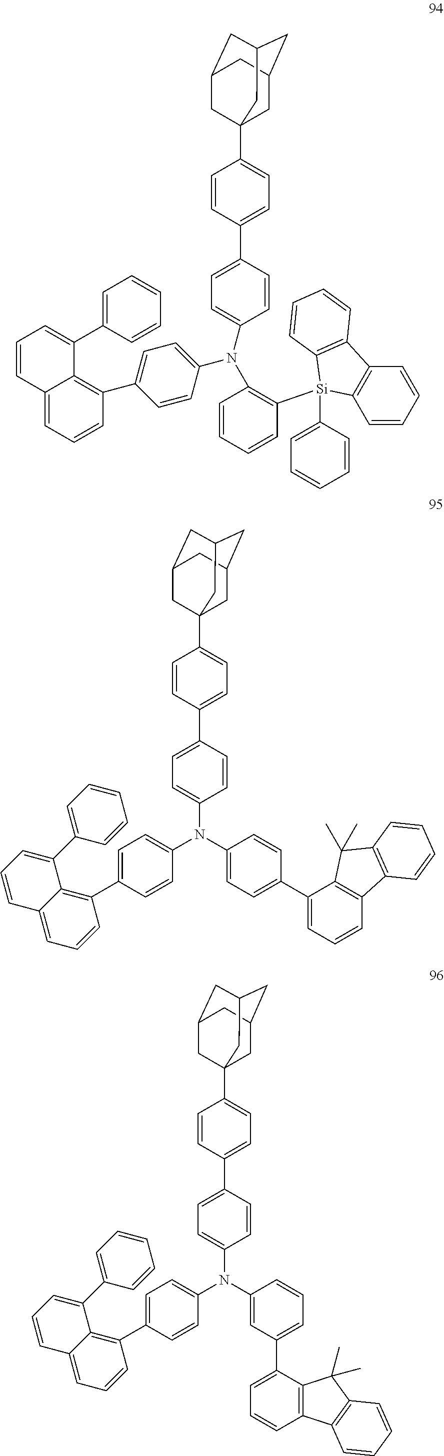 [SMILES: c1ccc(-c2cccc3cccc(-c4ccc(N(c5ccc(-c6ccc(C78CC9CC(CC(C9)C7)C8)cc6)cc5)c5ccccc5[Si]5(c6ccccc6)c6ccccc6-c6ccccc65)cc4)c23)cc1 ; CC1(C)c2ccccc2-c2cccc(-c3ccc(N(c4ccc(-c5ccc(C67CC8CC(CC(C8)C6)C7)cc5)cc4)c4ccc(-c5cccc6cccc(-c7ccccc7)c56)cc4)cc3)c21 ; CC1(C)c2ccccc2-c2cccc(-c3cccc(N(c4ccc(-c5ccc(C67CC8CC(CC(C8)C6)C7)cc5)cc4)c4ccc(-c5cccc6cccc(-c7ccccc7)c56)cc4)c3)c21]

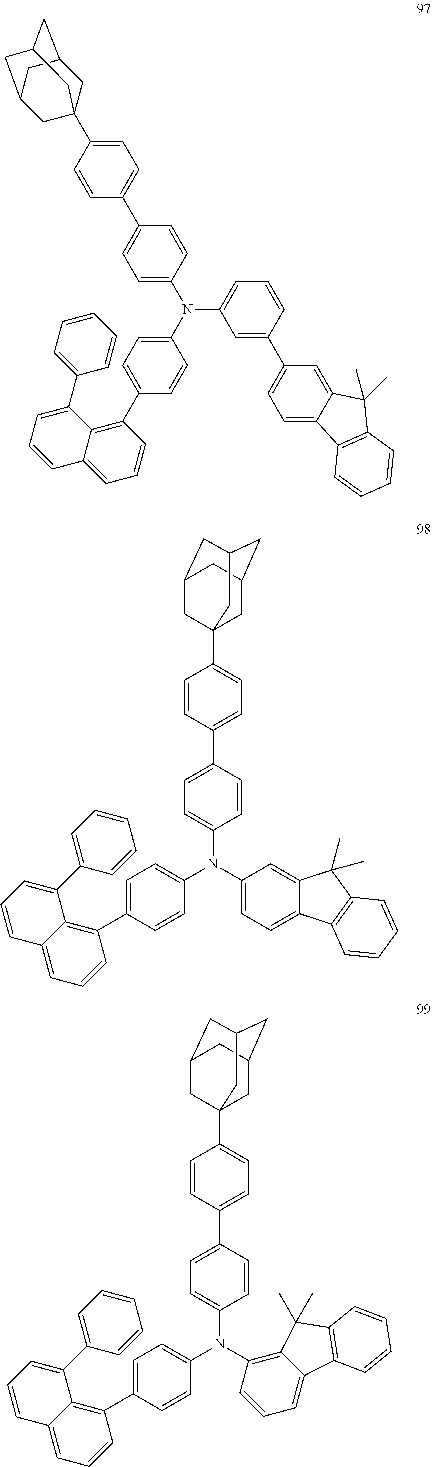 [SMILES: CC1(C)c2ccccc2-c2ccc(-c3cccc(N(c4ccc(-c5ccc(C67CC8CC(CC(C8)C6)C7)cc5)cc4)c4ccc(-c5cccc6cccc(-c7ccccc7)c56)cc4)c3)cc21 ; CC1(C)c2ccccc2-c2ccc(N(c3ccc(-c4ccc(C56CC7CC(CC(C7)C5)C6)cc4)cc3)c3ccc(-c4cccc5cccc(-c6ccccc6)c45)cc3)cc21 ; CC1(C)c2ccccc2-c2cccc(N(c3ccc(-c4ccc(C56CC7CC(CC(C7)C5)C6)cc4)cc3)c3ccc(-c4cccc5cccc(-c6ccccc6)c45)cc3)c21]

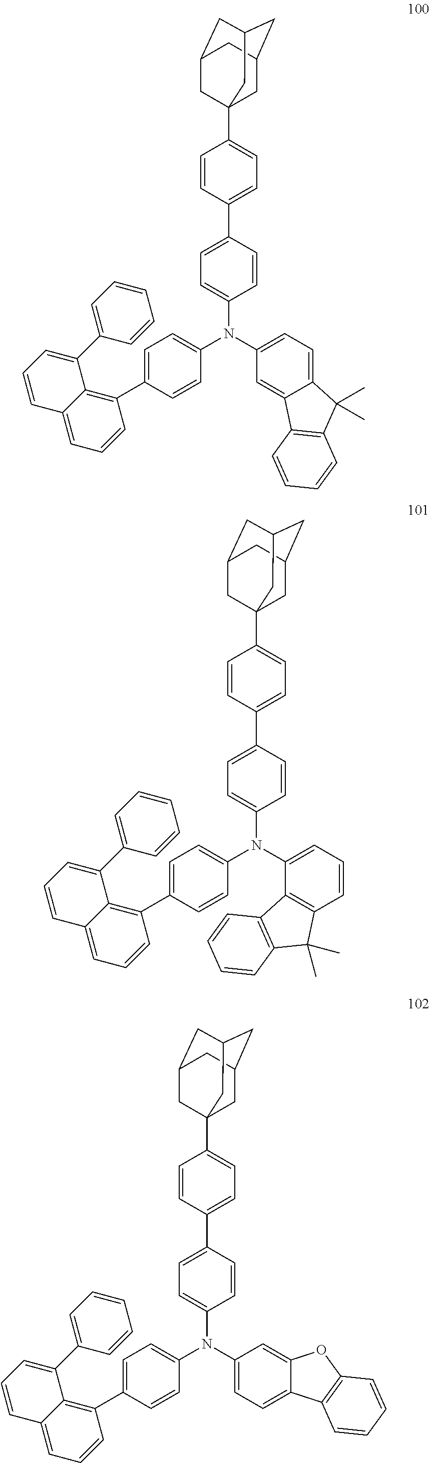

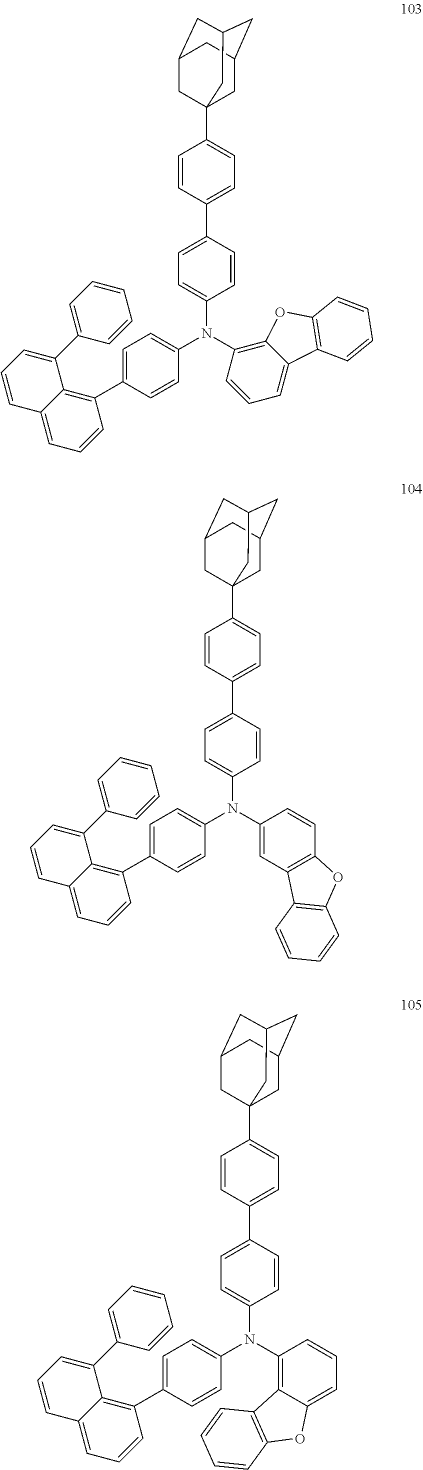 [SMILES: c1ccc(-c2cccc3cccc(-c4ccc(N(c5ccc(-c6ccc(C78CC9CC(CC(C9)C7)C8)cc6)cc5)c5cccc6c5oc5ccccc56)cc4)c23)cc1 ; c1ccc(-c2cccc3cccc(-c4ccc(N(c5ccc(-c6ccc(C78CC9CC(CC(C9)C7)C8)cc6)cc5)c5ccc6oc7ccccc7c6c5)cc4)c23)cc1 ; c1ccc(-c2cccc3cccc(-c4ccc(N(c5ccc(-c6ccc(C78CC9CC(CC(C9)C7)C8)cc6)cc5)c5cccc6oc7ccccc7c56)cc4)c23)cc1]

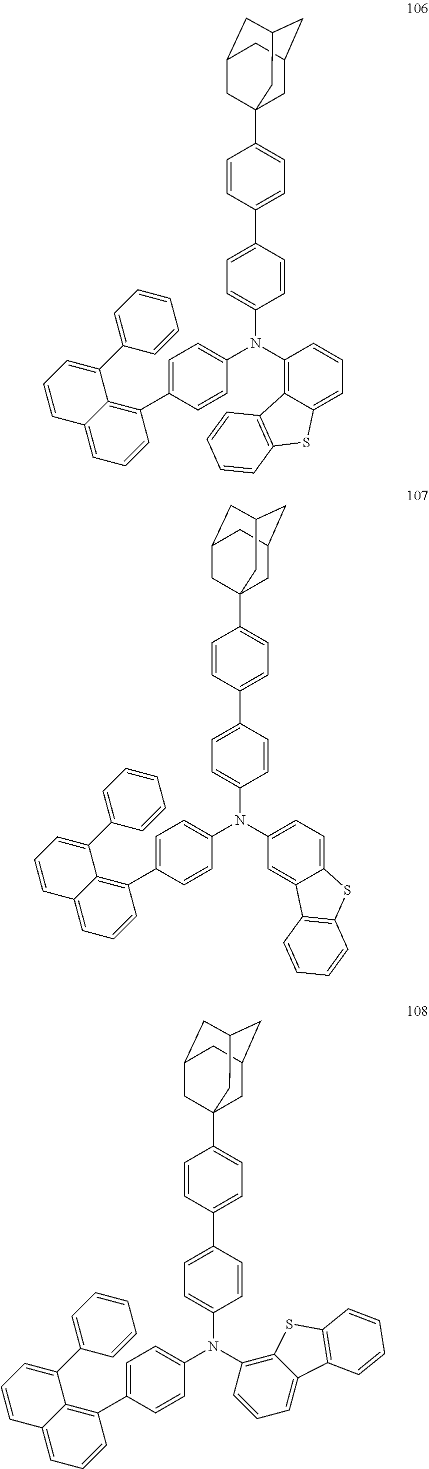

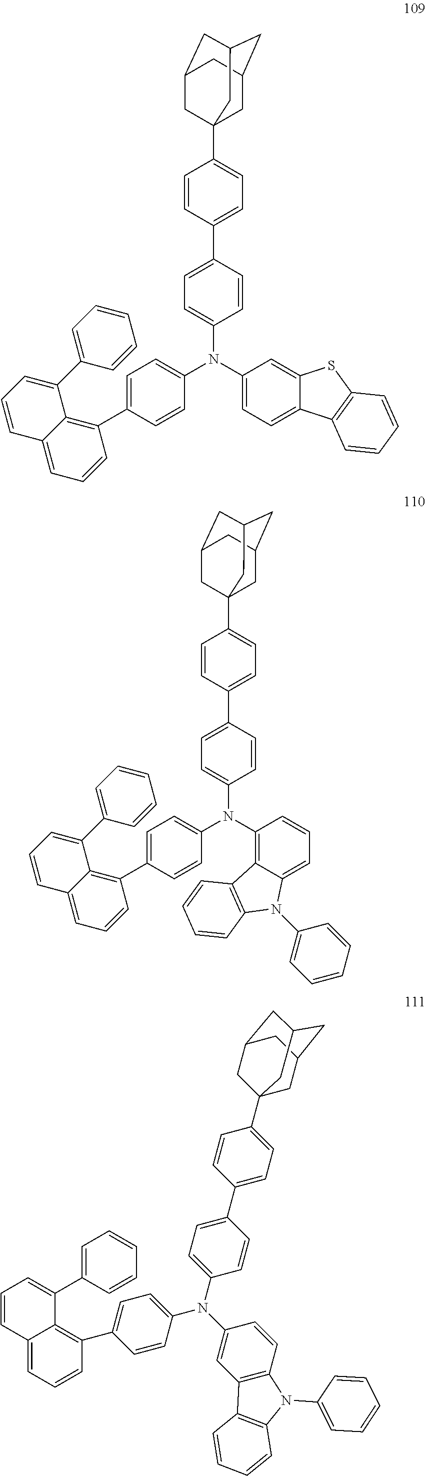

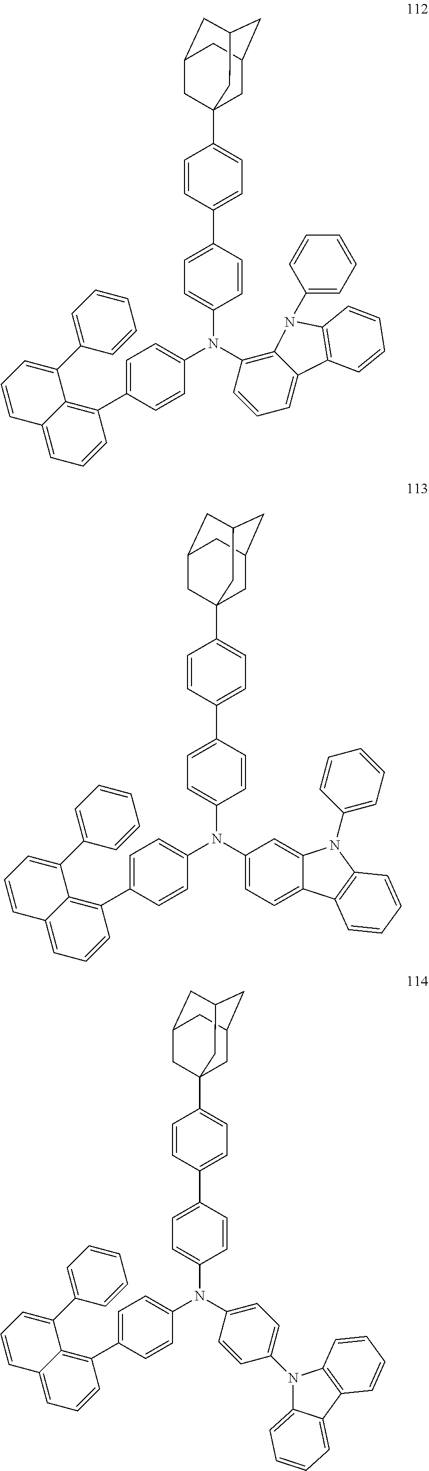 [SMILES: c1ccc(-c2cccc3cccc(-c4ccc(N(c5ccc(-c6ccc(C78CC9CC(CC(C9)C7)C8)cc6)cc5)c5cccc6c7ccccc7n(-c7ccccc7)c56)cc4)c23)cc1 ; c1ccc(-c2cccc3cccc(-c4ccc(N(c5ccc(-c6ccc(C78CC9CC(CC(C9)C7)C8)cc6)cc5)c5ccc6c7ccccc7n(-c7ccccc7)c6c5)cc4)c23)cc1 ; c1ccc(-c2cccc3cccc(-c4ccc(N(c5ccc(-c6ccc(C78CC9CC(CC(C9)C7)C8)cc6)cc5)c5ccc(-n6c7ccccc7c7ccccc76)cc5)cc4)c23)cc1]

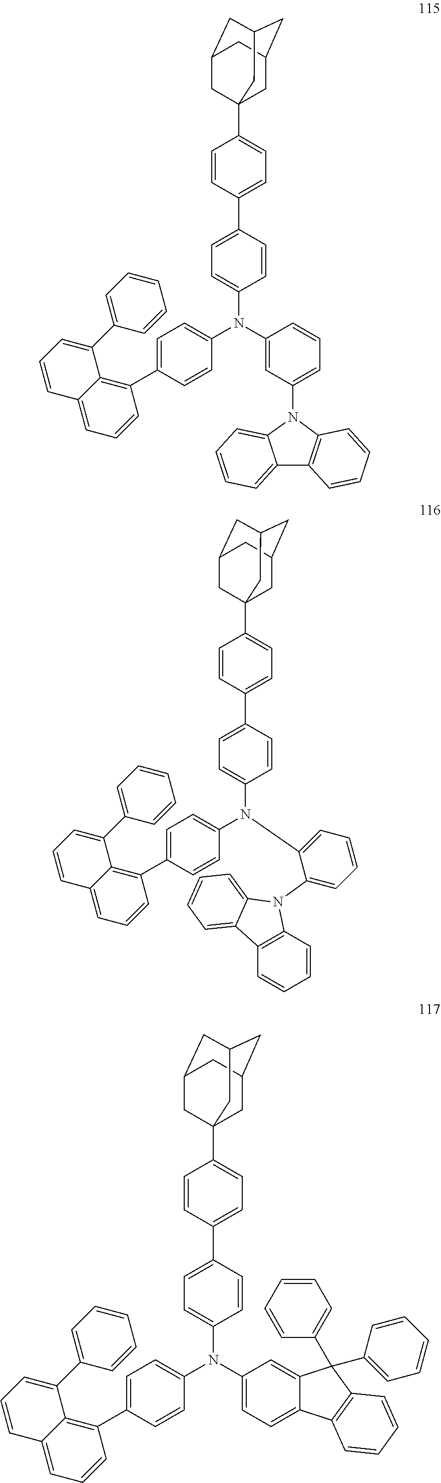

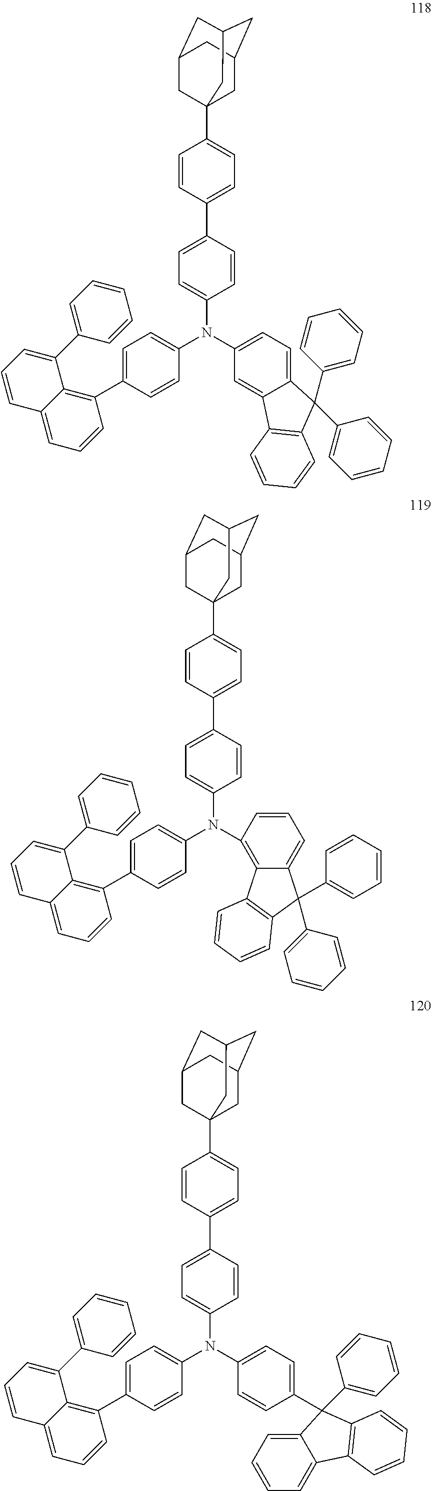

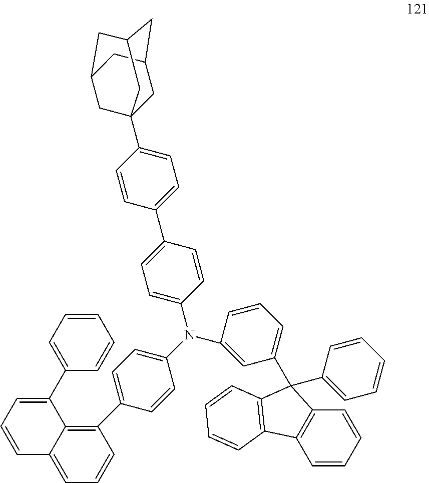 [SMILES: c1ccc(-c2cccc3cccc(-c4ccc(N(c5ccc(-c6ccc(C78CC9CC(CC(C9)C7)C8)cc6)cc5)c5cccc(C6(c7ccccc7)c7ccccc7-c7ccccc76)c5)cc4)c23)cc1]

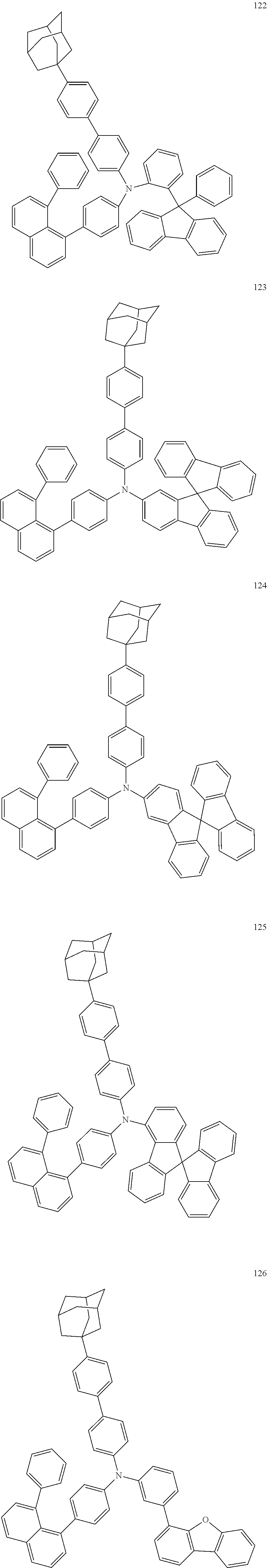

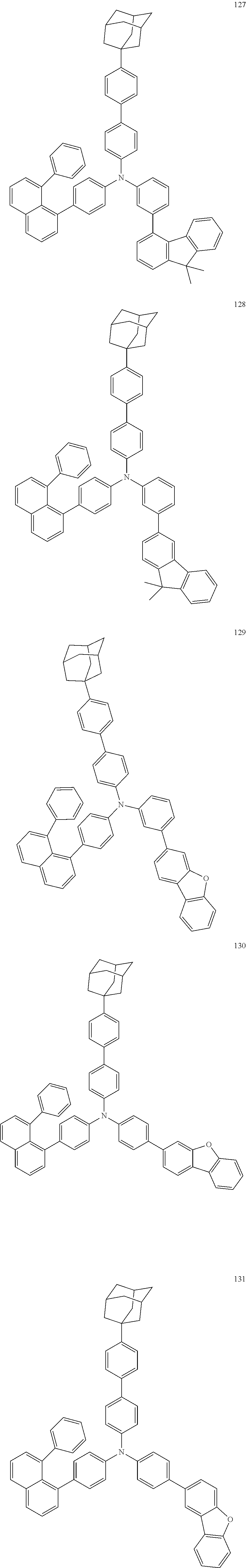

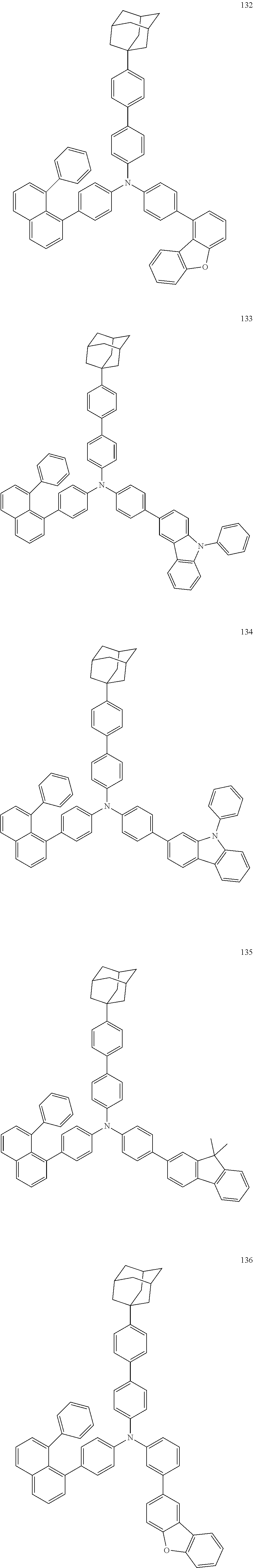

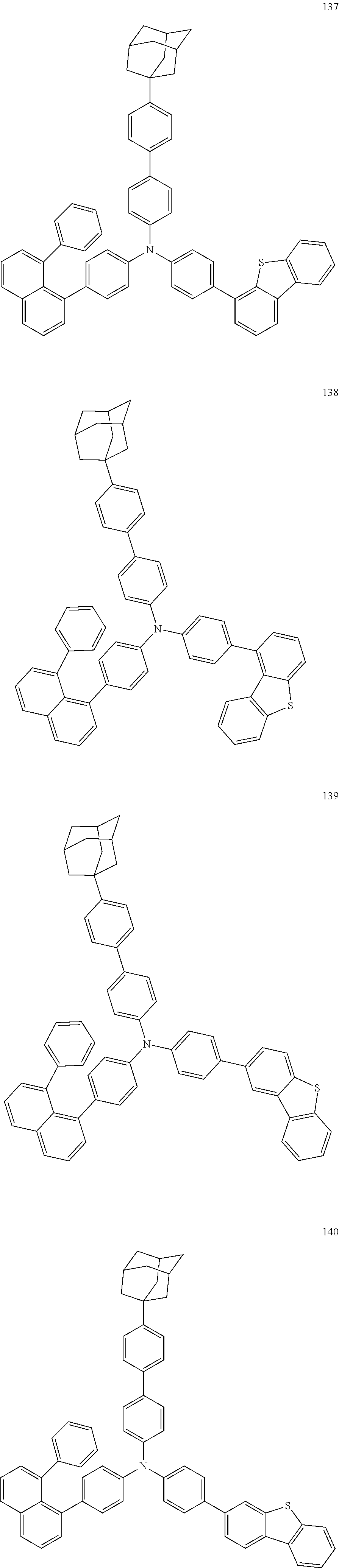 [SMILES: c1ccc(-c2cccc3cccc(-c4ccc(N(c5ccc(-c6ccc(C78CC9CC(CC(C9)C7)C8)cc6)cc5)c5ccc(-c6cccc7c6sc6ccccc67)cc5)cc4)c23)cc1 ; c1ccc(-c2cccc3cccc(-c4ccc(N(c5ccc(-c6ccc(C78CC9CC(CC(C9)C7)C8)cc6)cc5)c5ccc(-c6cccc7sc8ccccc8c67)cc5)cc4)c23)cc1 ; c1ccc(-c2cccc3cccc(-c4ccc(N(c5ccc(-c6ccc(C78CC9CC(CC(C9)C7)C8)cc6)cc5)c5ccc(-c6ccc7sc8ccccc8c7c6)cc5)cc4)c23)cc1 ; c1ccc(-c2cccc3cccc(-c4ccc(N(c5ccc(-c6ccc(C78CC9CC(CC(C9)C7)C8)cc6)cc5)c5ccc(-c6ccc7c(c6)sc6ccccc67)cc5)cc4)c23)cc1]

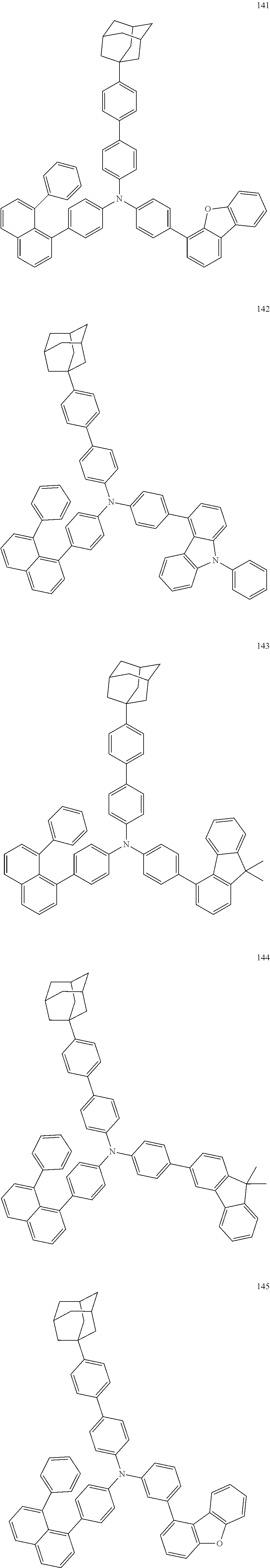

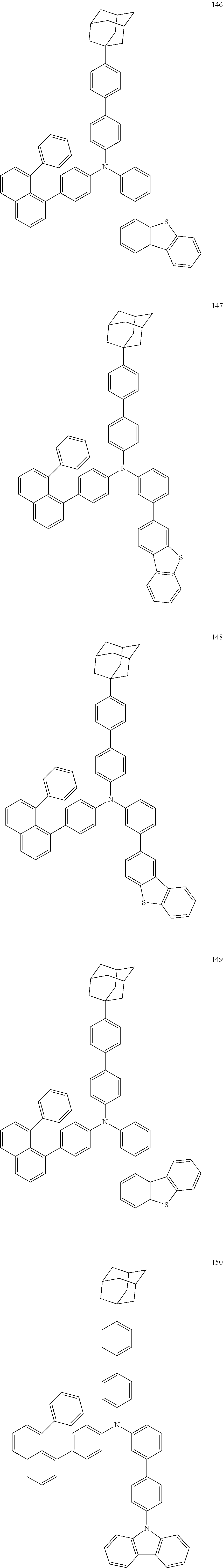

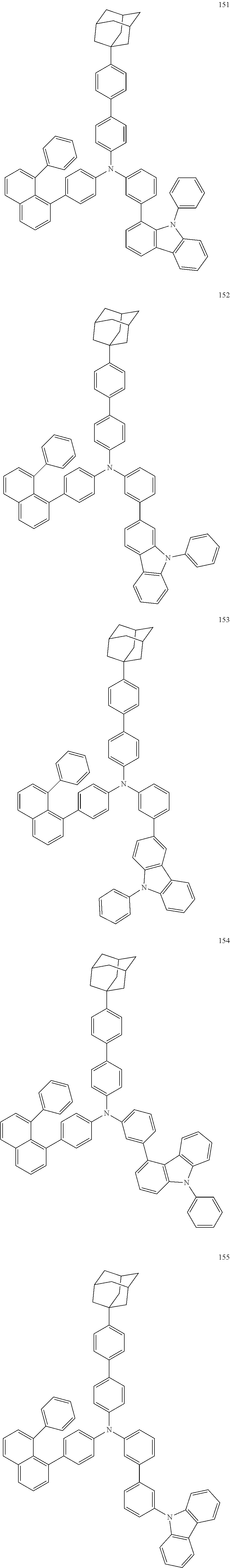

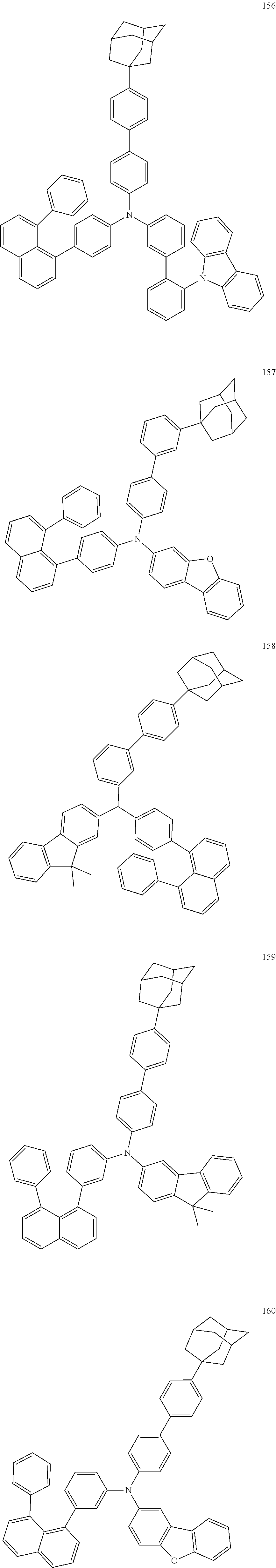

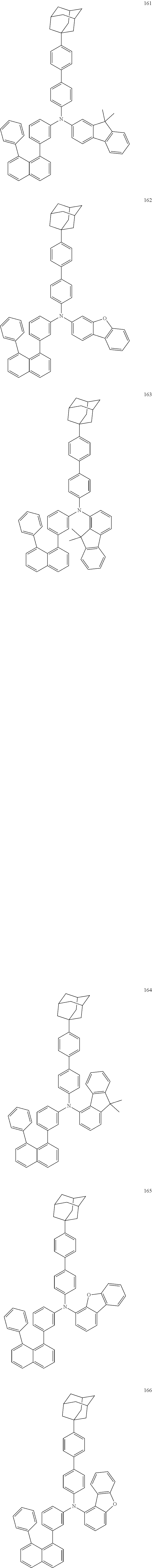

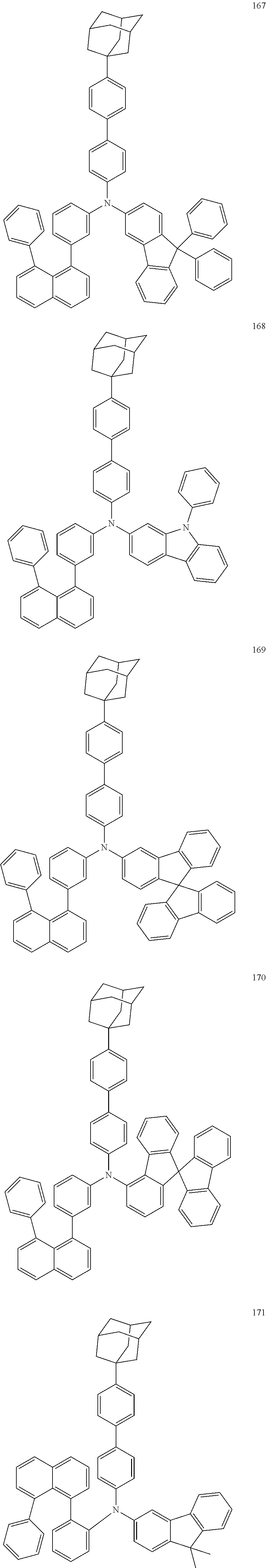

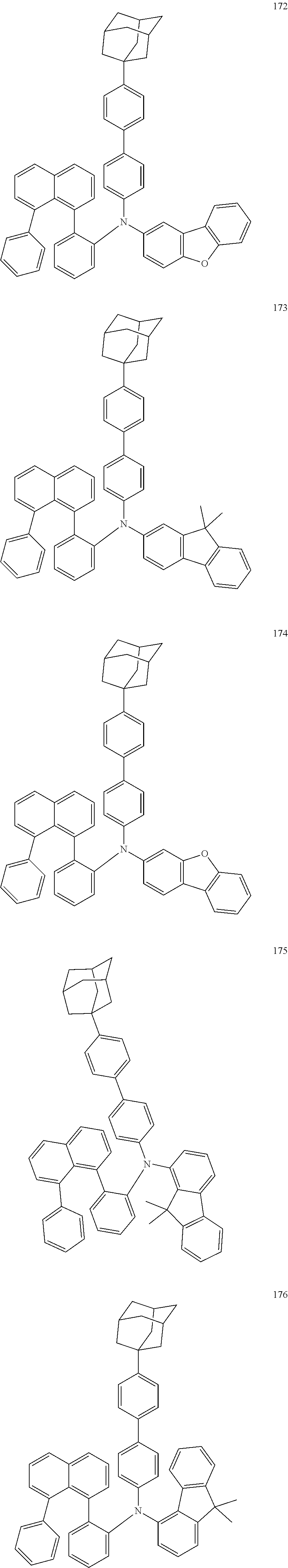

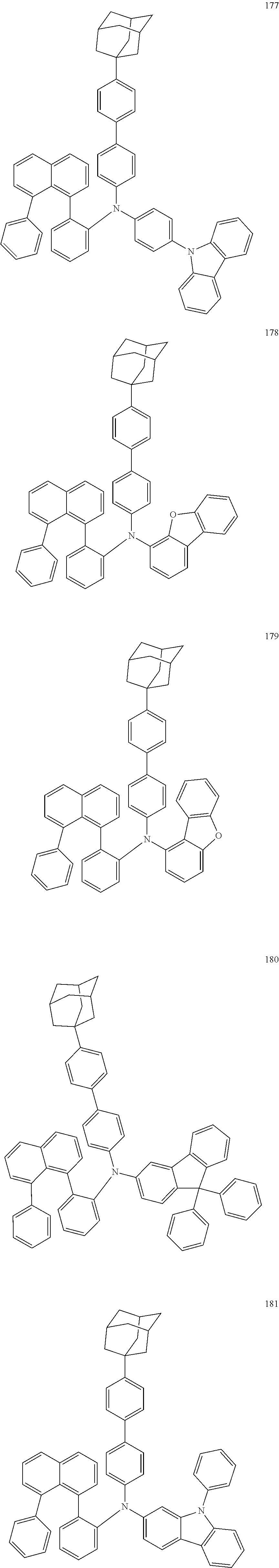

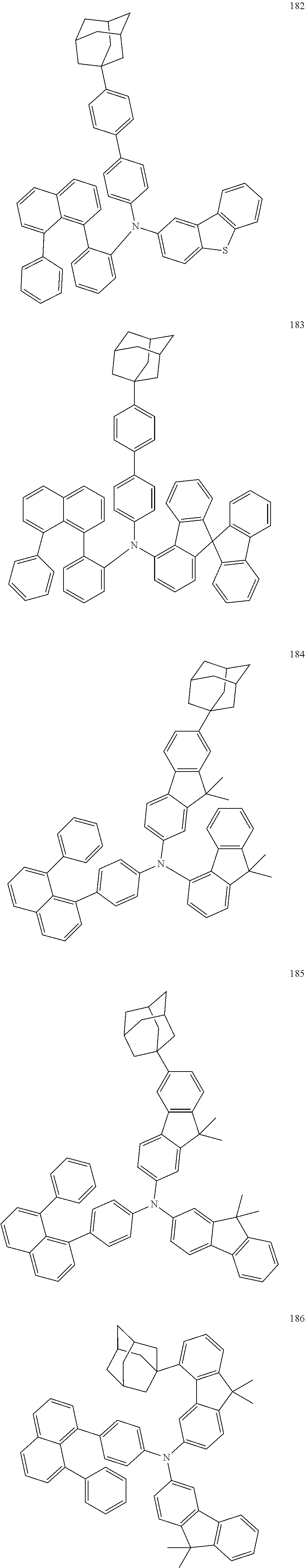

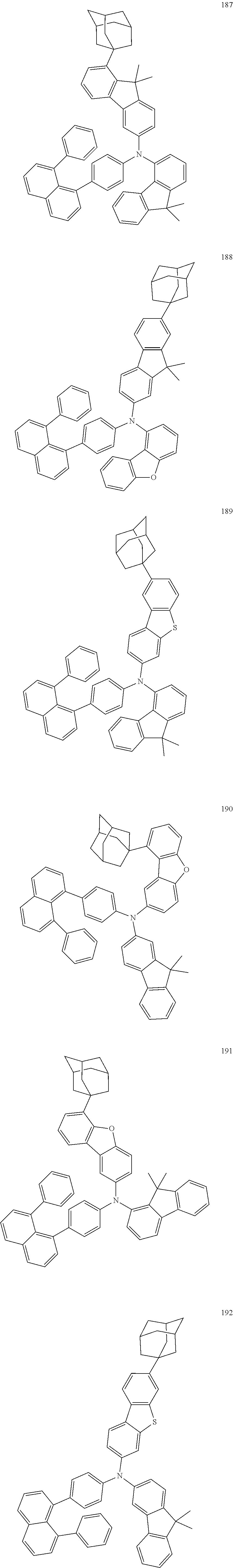

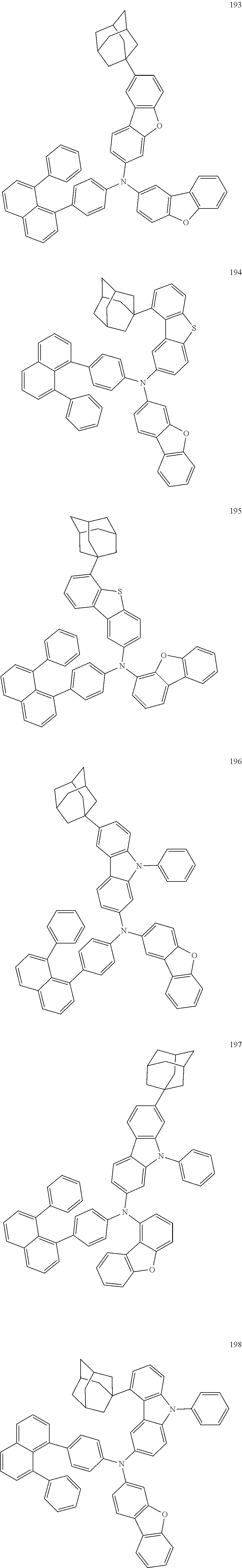

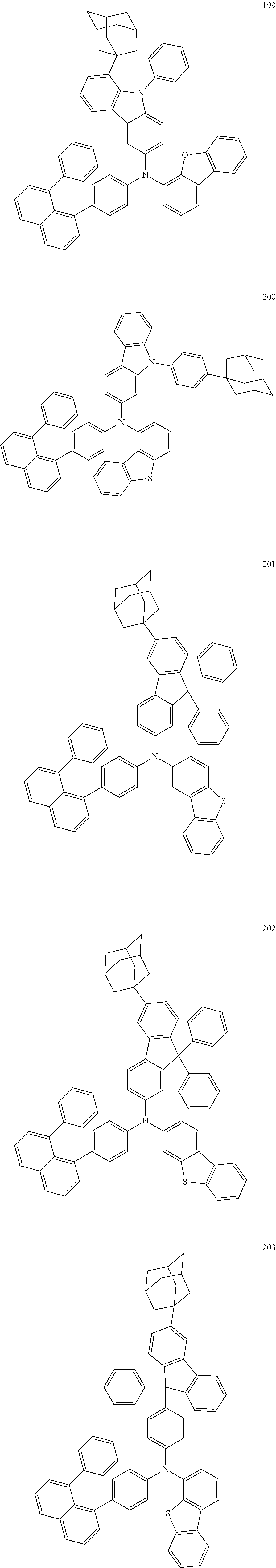

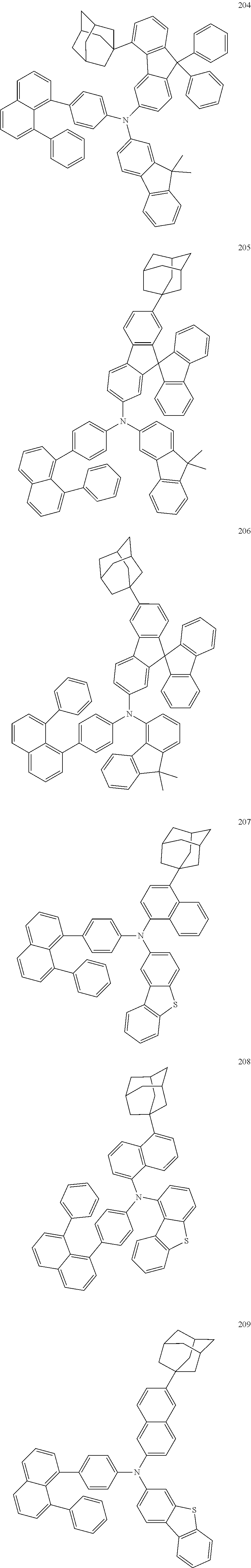

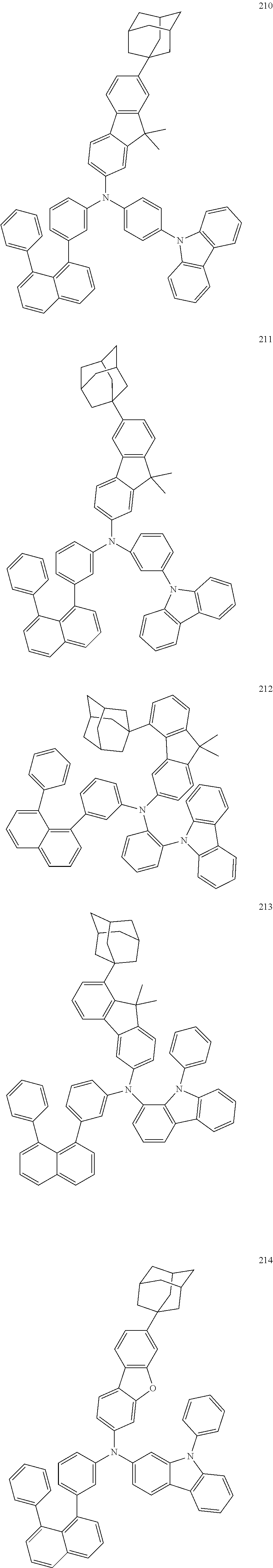

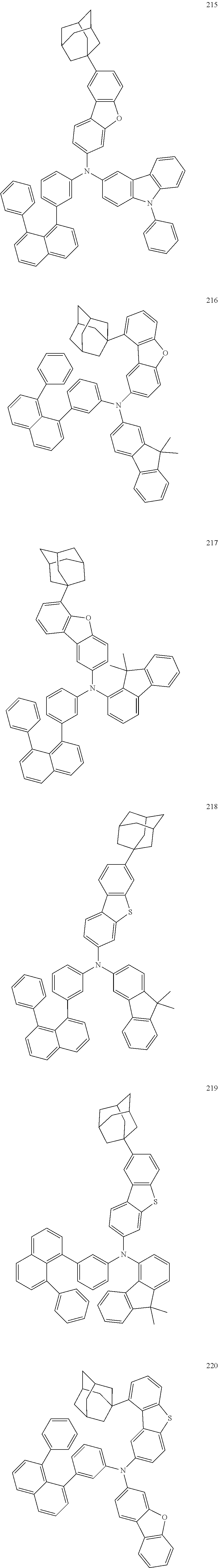

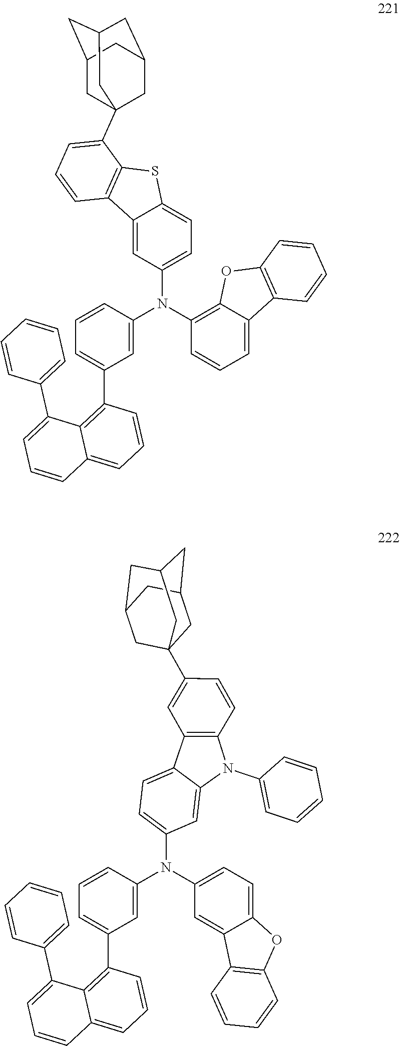 [SMILES: c1ccc(-c2cccc3cccc(-c4cccc(N(c5ccc6sc7c(C89CC%10CC(CC(C%10)C8)C9)cccc7c6c5)c5cccc6c5oc5ccccc56)c4)c23)cc1 ; c1ccc(-c2cccc3cccc(-c4cccc(N(c5ccc6oc7ccccc7c6c5)c5ccc6c7cc(C89CC%10CC(CC(C%10)C8)C9)ccc7n(-c7ccccc7)c6c5)c4)c23)cc1]

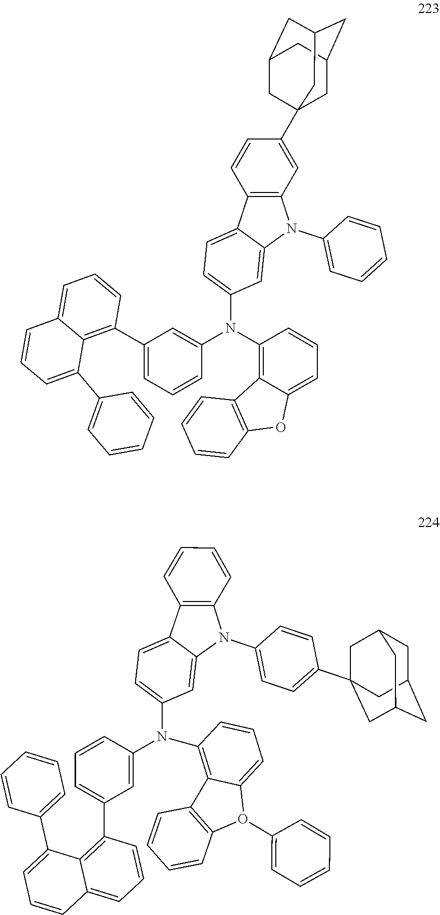

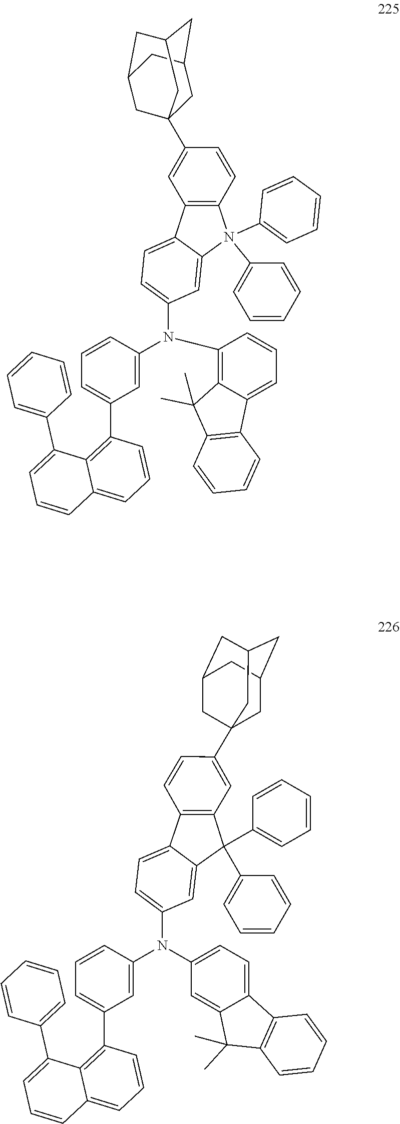

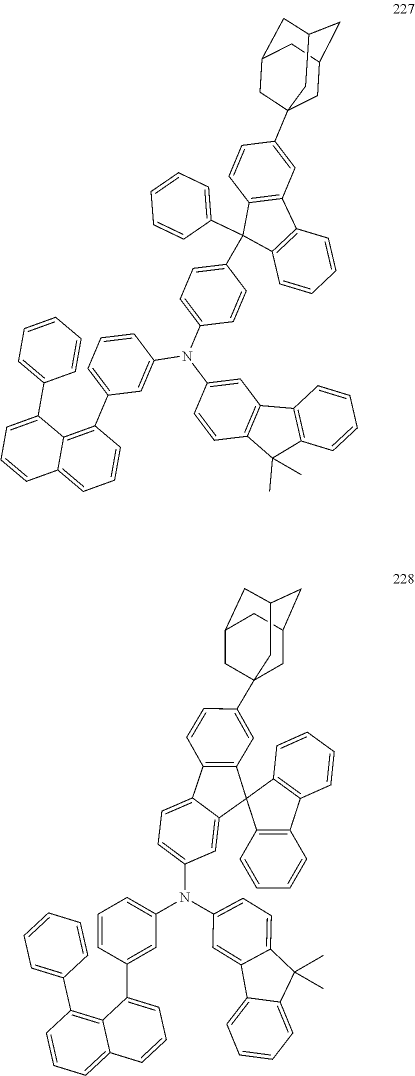 [SMILES: CC1(C)c2ccccc2-c2cc(N(c3ccc(C4(c5ccccc5)c5ccccc5-c5cc(C67CC8CC(CC(C8)C6)C7)ccc54)cc3)c3cccc(-c4cccc5cccc(-c6ccccc6)c45)c3)ccc21 ; CC1(C)c2ccccc2-c2cc(N(c3cccc(-c4cccc5cccc(-c6ccccc6)c45)c3)c3ccc4c(c3)C3(c5ccccc5-c5ccccc53)c3cc(C56CC7CC(CC(C7)C5)C6)ccc3-4)ccc21]

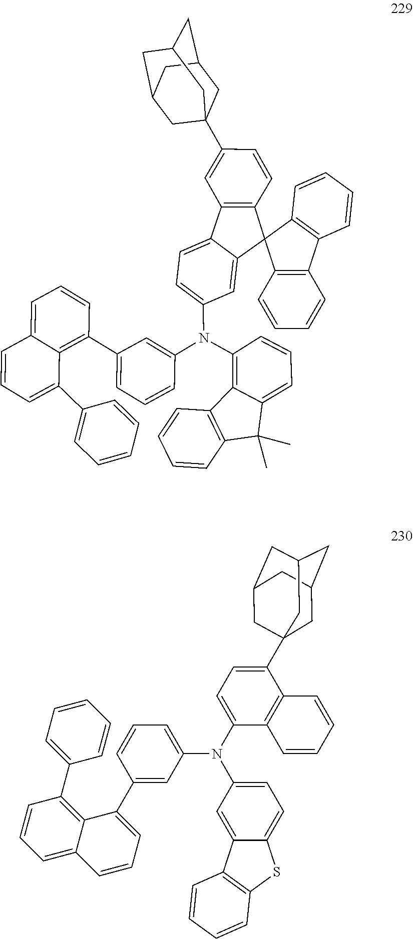 [SMILES: CC1(C)c2ccccc2-c2c(N(c3cccc(-c4cccc5cccc(-c6ccccc6)c45)c3)c3ccc4c(c3)C3(c5ccccc5-c5ccccc53)c3ccc(C56CC7CC(CC(C7)C5)C6)cc3-4)cccc21 ; c1ccc(-c2cccc3cccc(-c4cccc(N(c5ccc6sc7ccccc7c6c5)c5ccc(C67CC8CC(CC(C8)C6)C7)c6ccccc56)c4)c23)cc1]

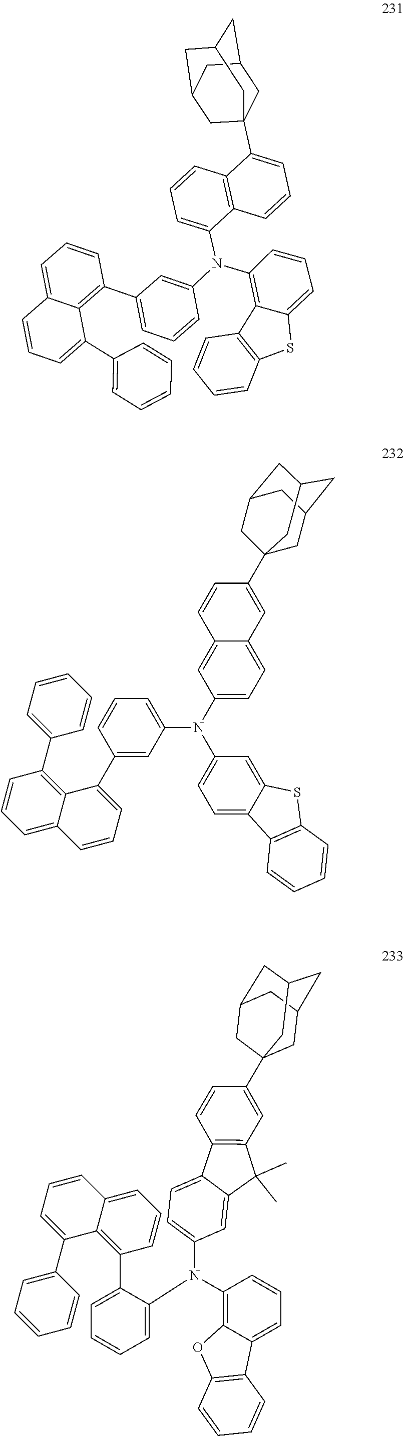 [SMILES: c1ccc(-c2cccc3cccc(-c4cccc(N(c5cccc6c(C78CC9CC(CC(C9)C7)C8)cccc56)c5cccc6sc7ccccc7c56)c4)c23)cc1 ; c1ccc(-c2cccc3cccc(-c4cccc(N(c5ccc6cc(C78CC9CC(CC(C9)C7)C8)ccc6c5)c5ccc6c(c5)sc5ccccc56)c4)c23)cc1 ; CC1(C)c2cc(N(c3ccccc3-c3cccc4cccc(-c5ccccc5)c34)c3cccc4c3oc3ccccc34)ccc2-c2ccc(C34CC5CC(CC(C5)C3)C4)cc21]

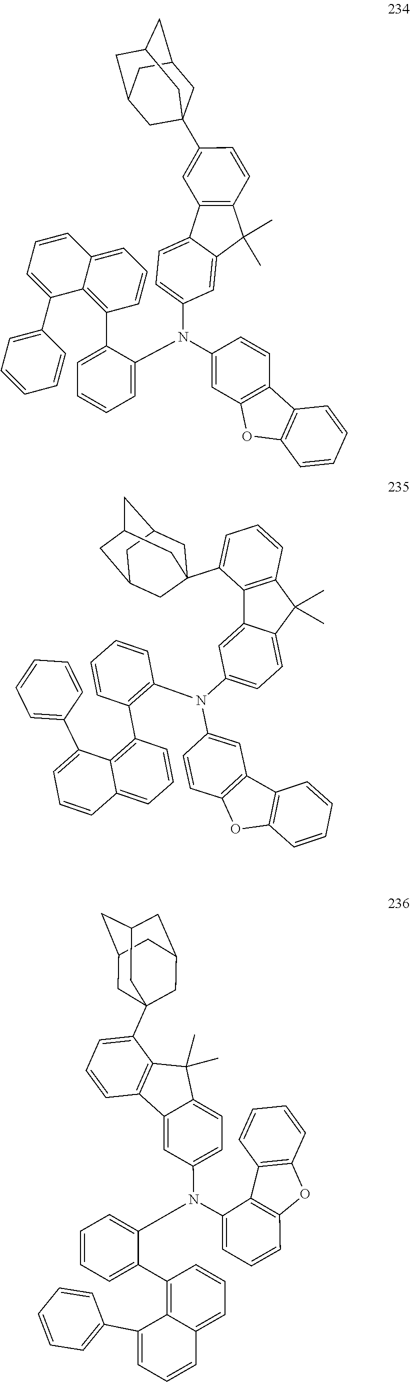 [SMILES: CC1(C)c2ccc(C34CC5CC(CC(C5)C3)C4)cc2-c2ccc(N(c3ccc4c(c3)oc3ccccc34)c3ccccc3-c3cccc4cccc(-c5ccccc5)c34)cc21 ; CC1(C)c2ccc(N(c3ccc4oc5ccccc5c4c3)c3ccccc3-c3cccc4cccc(-c5ccccc5)c34)cc2-c2c(C34CC5CC(CC(C5)C3)C4)cccc21 ; CC1(C)c2ccc(N(c3ccccc3-c3cccc4cccc(-c5ccccc5)c34)c3cccc4oc5ccccc5c34)cc2-c2cccc(C34CC5CC(CC(C5)C3)C4)c21]

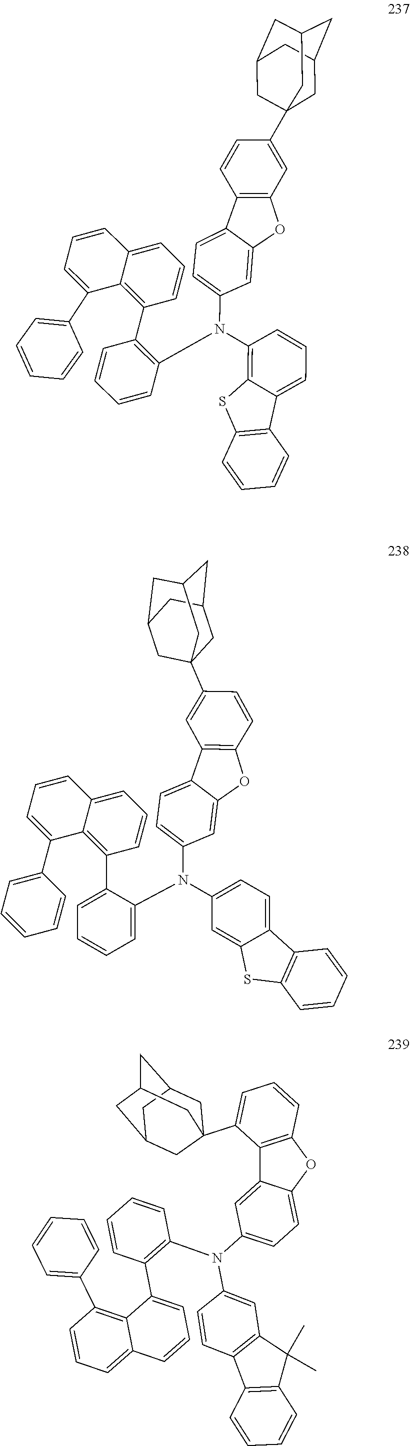 [SMILES: c1ccc(-c2cccc3cccc(-c4ccccc4N(c4ccc5c(c4)oc4cc(C67CC8CC(CC(C8)C6)C7)ccc45)c4cccc5c4sc4ccccc45)c23)cc1 ; c1ccc(-c2cccc3cccc(-c4ccccc4N(c4ccc5c(c4)oc4ccc(C67CC8CC(CC(C8)C6)C7)cc45)c4ccc5c(c4)sc4ccccc45)c23)cc1 ; CC1(C)c2ccccc2-c2ccc(N(c3ccc4oc5cccc(C67CC8CC(CC(C8)C6)C7)c5c4c3)c3ccccc3-c3cccc4cccc(-c5ccccc5)c34)cc21]

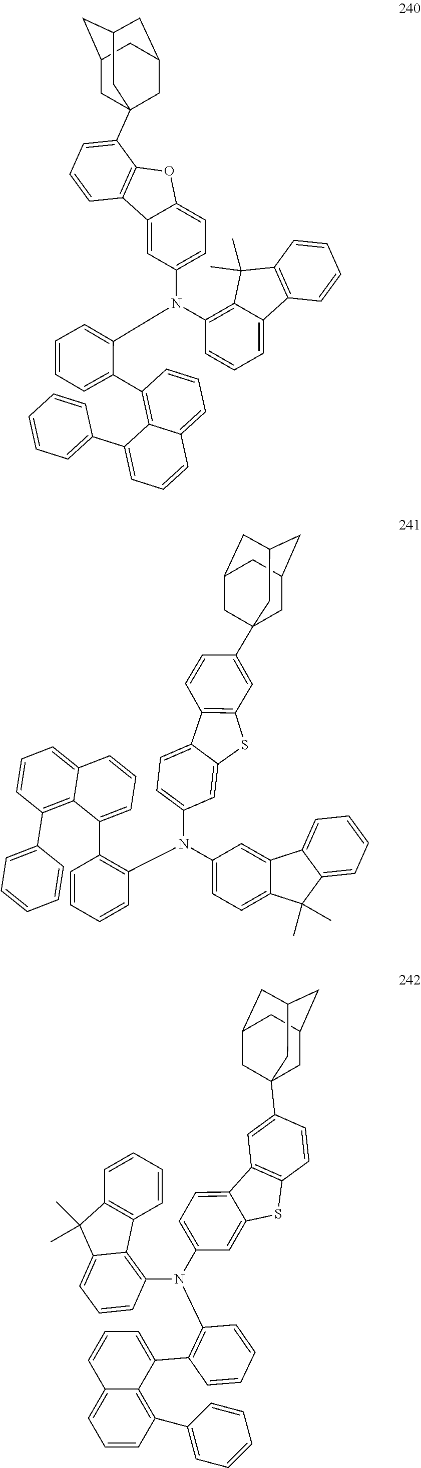 [SMILES: CC1(C)c2ccccc2-c2cccc(N(c3ccc4oc5c(C67CC8CC(CC(C8)C6)C7)cccc5c4c3)c3ccccc3-c3cccc4cccc(-c5ccccc5)c34)c21 ; CC1(C)c2ccccc2-c2cc(N(c3ccc4c(c3)sc3cc(C56CC7CC(CC(C7)C5)C6)ccc34)c3ccccc3-c3cccc4cccc(-c5ccccc5)c34)ccc21 ; CC1(C)c2ccccc2-c2c(N(c3ccc4c(c3)sc3ccc(C56CC7CC(CC(C7)C5)C6)cc34)c3ccccc3-c3cccc4cccc(-c5ccccc5)c34)cccc21]

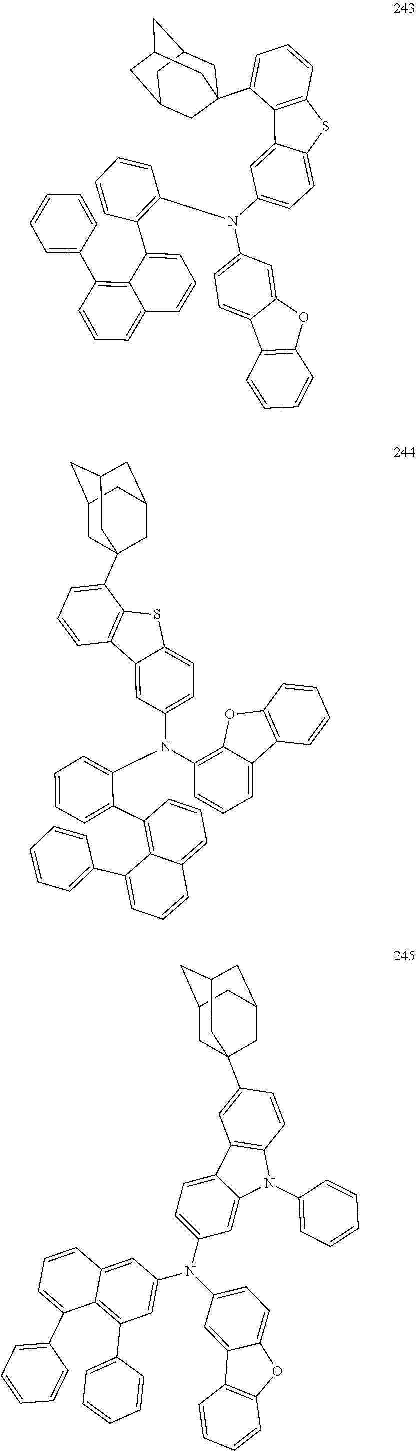 [SMILES: c1ccc(-c2cccc3cccc(-c4ccccc4N(c4ccc5c(c4)oc4ccccc45)c4ccc5sc6cccc(C78CC9CC(CC(C9)C7)C8)c6c5c4)c23)cc1 ; c1ccc(-c2cccc3cccc(-c4ccccc4N(c4ccc5sc6c(C78CC9CC(CC(C9)C7)C8)cccc6c5c4)c4cccc5c4oc4ccccc45)c23)cc1 ; c1ccc(-c2cccc3cc(N(c4ccc5oc6ccccc6c5c4)c4ccc5c6cc(C78CC9CC(CC(C9)C7)C8)ccc6n(-c6ccccc6)c5c4)cc(-c4ccccc4)c23)cc1]

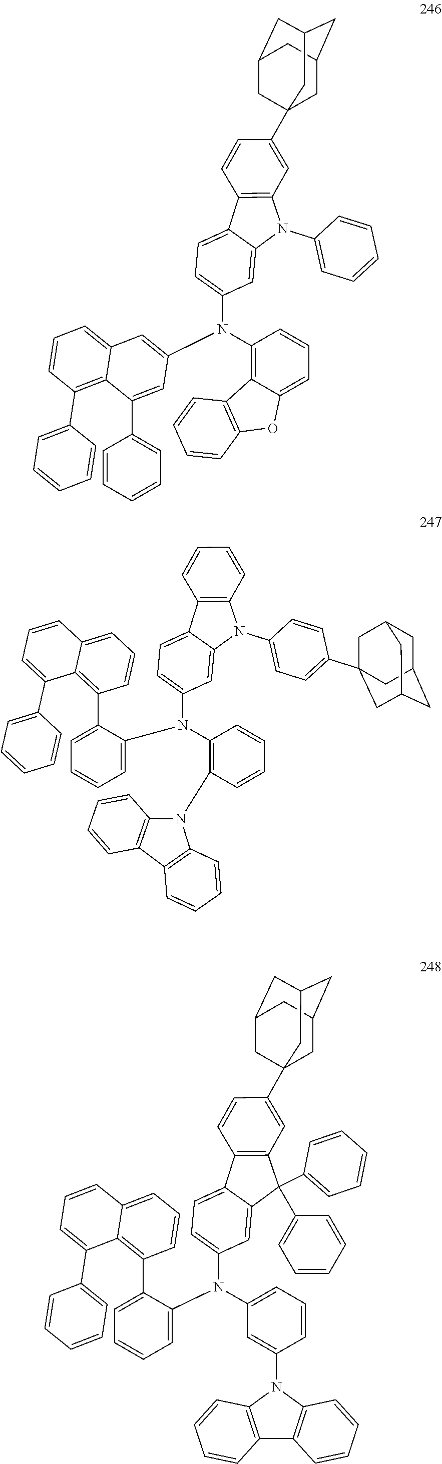 [SMILES: c1ccc(-c2cccc3cc(N(c4ccc5c6ccc(C78CC9CC(CC(C9)C7)C8)cc6n(-c6ccccc6)c5c4)c4cccc5oc6ccccc6c45)cc(-c4ccccc4)c23)cc1 ; c1ccc(-c2cccc3cccc(-c4ccccc4N(c4ccc5c6ccccc6n(-c6ccc(C78CC9CC(CC(C9)C7)C8)cc6)c5c4)c4ccccc4-n4c5ccccc5c5ccccc54)c23)cc1 ; c1ccc(-c2cccc3cccc(-c4ccccc4N(c4cccc(-n5c6ccccc6c6ccccc65)c4)c4ccc5c(c4)C(c4ccccc4)(c4ccccc4)c4cc(C67CC8CC(CC(C8)C6)C7)ccc4-5)c23)cc1]

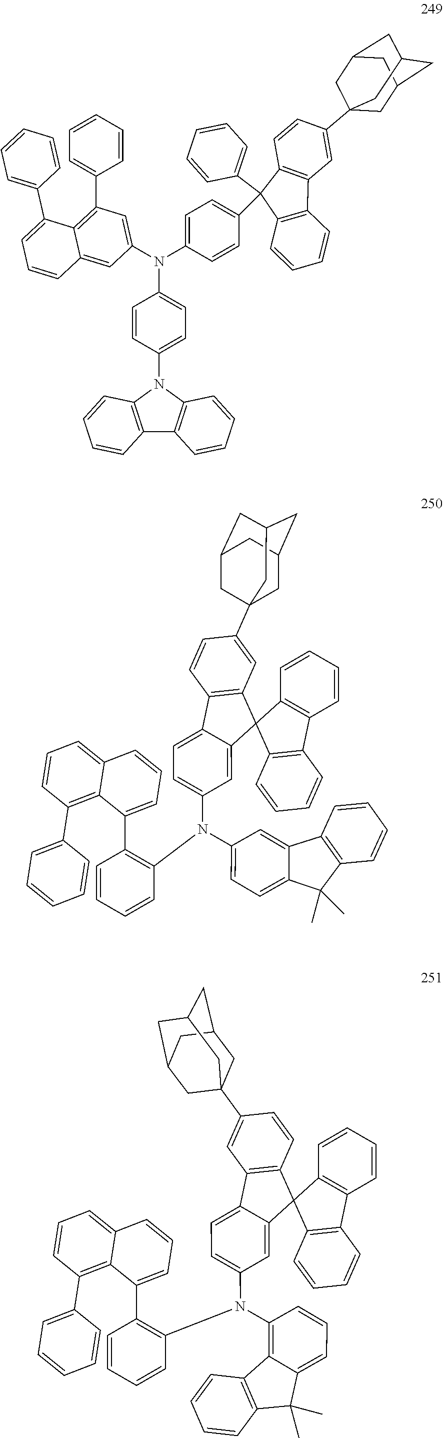 [SMILES: c1ccc(-c2cccc3cc(N(c4ccc(-n5c6ccccc6c6ccccc65)cc4)c4ccc(C5(c6ccccc6)c6ccccc6-c6cc(C78CC9CC(CC(C9)C7)C8)ccc65)cc4)cc(-c4ccccc4)c23)cc1 ; CC1(C)c2ccccc2-c2cc(N(c3ccc4c(c3)C3(c5ccccc5-c5ccccc53)c3cc(C56CC7CC(CC(C7)C5)C6)ccc3-4)c3ccccc3-c3cccc4cccc(-c5ccccc5)c34)ccc21 ; CC1(C)c2ccccc2-c2c(N(c3ccc4c(c3)C3(c5ccccc5-c5ccccc53)c3ccc(C56CC7CC(CC(C7)C5)C6)cc3-4)c3ccccc3-c3cccc4cccc(-c5ccccc5)c34)cccc21]

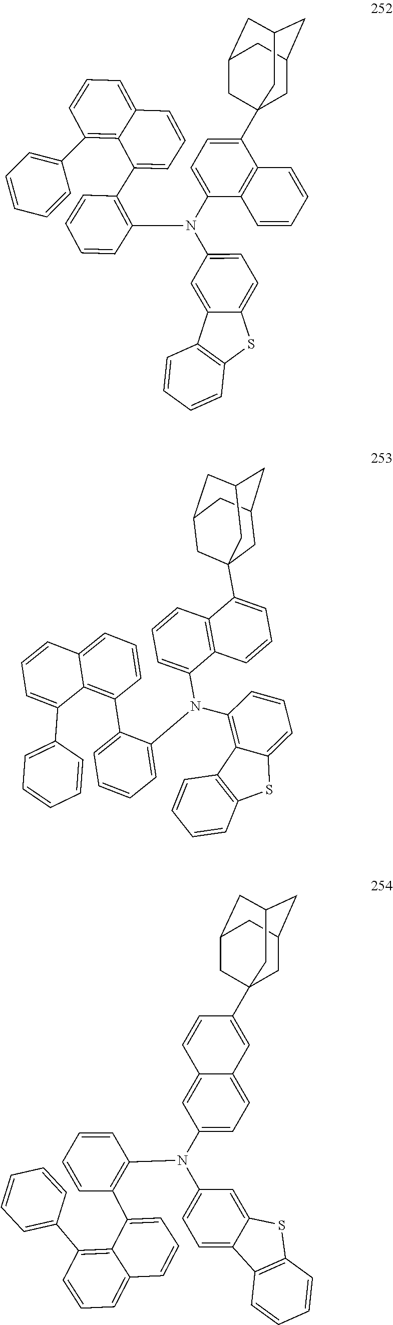 [SMILES: c1ccc(-c2cccc3cccc(-c4ccccc4N(c4ccc5sc6ccccc6c5c4)c4ccc(C56CC7CC(CC(C7)C5)C6)c5ccccc45)c23)cc1 ; c1ccc(-c2cccc3cccc(-c4ccccc4N(c4cccc5c(C67CC8CC(CC(C8)C6)C7)cccc45)c4cccc5sc6ccccc6c45)c23)cc1 ; c1ccc(-c2cccc3cccc(-c4ccccc4N(c4ccc5cc(C67CC8CC(CC(C8)C6)C7)ccc5c4)c4ccc5c(c4)sc4ccccc45)c23)cc1]

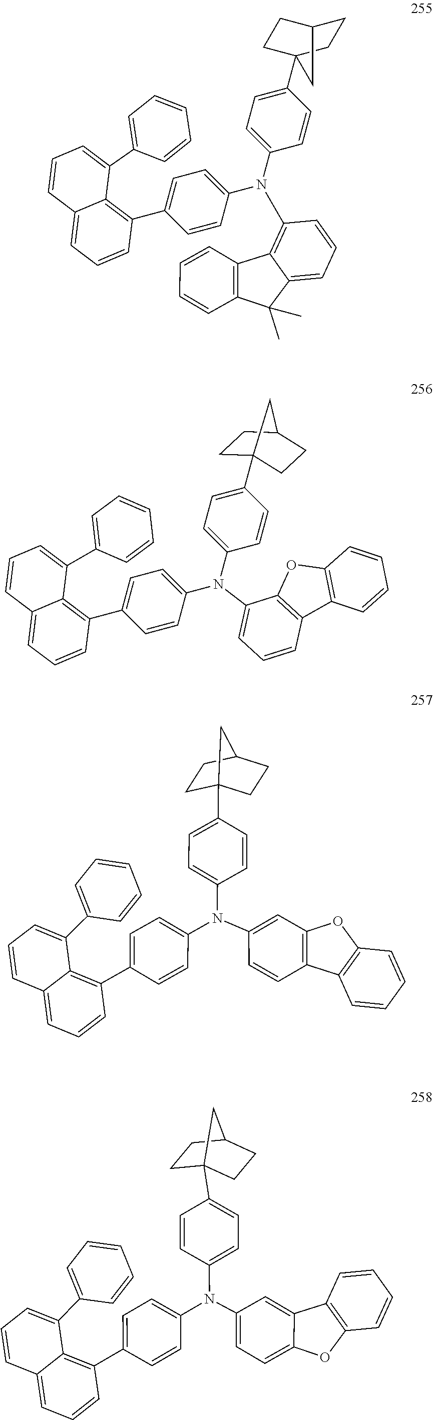 [SMILES: CC1(C)c2ccccc2-c2c(N(c3ccc(-c4cccc5cccc(-c6ccccc6)c45)cc3)c3ccc(C45CCC(CC4)C5)cc3)cccc21 ; c1ccc(-c2cccc3cccc(-c4ccc(N(c5ccc(C67CCC(CC6)C7)cc5)c5cccc6c5oc5ccccc56)cc4)c23)cc1 ; c1ccc(-c2cccc3cccc(-c4ccc(N(c5ccc(C67CCC(CC6)C7)cc5)c5ccc6c(c5)oc5ccccc56)cc4)c23)cc1 ; c1ccc(-c2cccc3cccc(-c4ccc(N(c5ccc(C67CCC(CC6)C7)cc5)c5ccc6oc7ccccc7c6c5)cc4)c23)cc1]

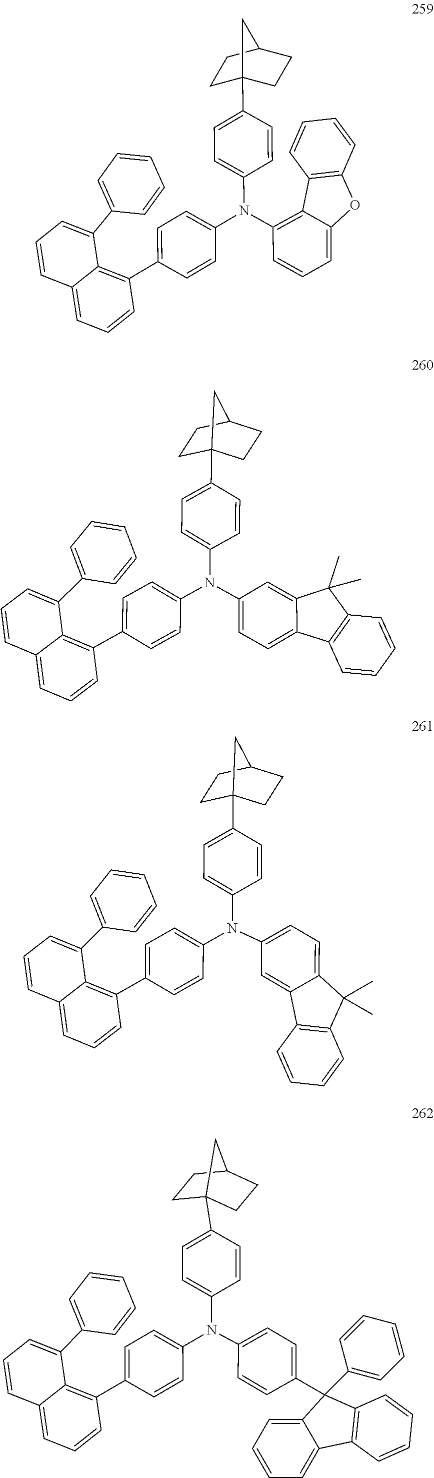 [SMILES: c1ccc(-c2cccc3cccc(-c4ccc(N(c5ccc(C67CCC(CC6)C7)cc5)c5cccc6oc7ccccc7c56)cc4)c23)cc1 ; CC1(C)c2ccccc2-c2ccc(N(c3ccc(-c4cccc5cccc(-c6ccccc6)c45)cc3)c3ccc(C45CCC(CC4)C5)cc3)cc21 ; CC1(C)c2ccccc2-c2cc(N(c3ccc(-c4cccc5cccc(-c6ccccc6)c45)cc3)c3ccc(C45CCC(CC4)C5)cc3)ccc21 ; c1ccc(-c2cccc3cccc(-c4ccc(N(c5ccc(C67CCC(CC6)C7)cc5)c5ccc(C6(c7ccccc7)c7ccccc7-c7ccccc76)cc5)cc4)c23)cc1]

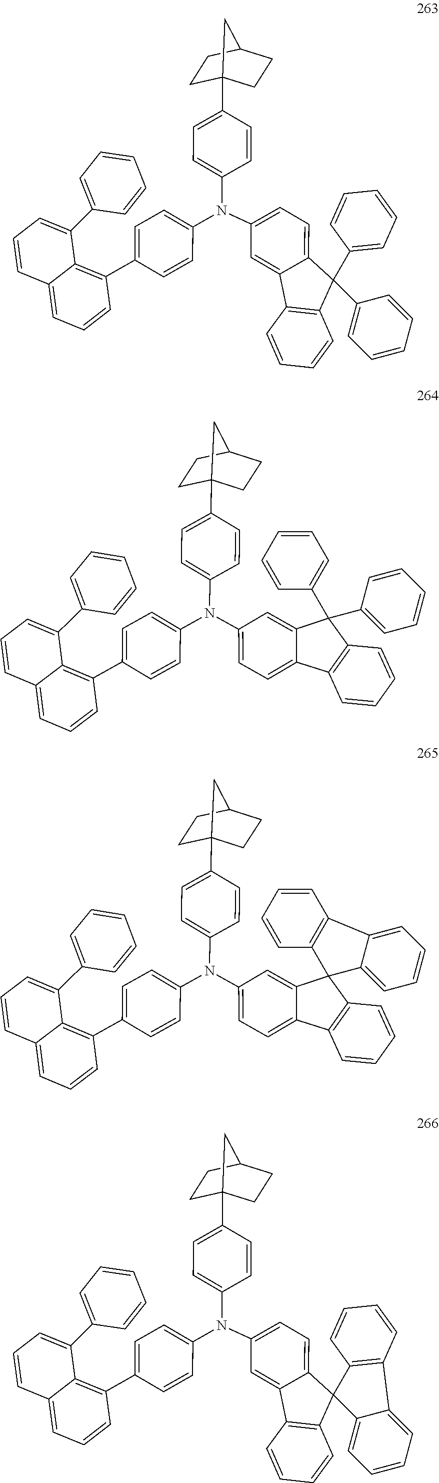 [SMILES: c1ccc(-c2cccc3cccc(-c4ccc(N(c5ccc(C67CCC(CC6)C7)cc5)c5ccc6c(c5)-c5ccccc5C6(c5ccccc5)c5ccccc5)cc4)c23)cc1 ; c1ccc(-c2cccc3cccc(-c4ccc(N(c5ccc(C67CCC(CC6)C7)cc5)c5ccc6c(c5)C(c5ccccc5)(c5ccccc5)c5ccccc5-6)cc4)c23)cc1 ; c1ccc(-c2cccc3cccc(-c4ccc(N(c5ccc(C67CCC(CC6)C7)cc5)c5ccc6c(c5)C5(c7ccccc7-c7ccccc75)c5ccccc5-6)cc4)c23)cc1 ; c1ccc(-c2cccc3cccc(-c4ccc(N(c5ccc(C67CCC(CC6)C7)cc5)c5ccc6c(c5)-c5ccccc5C65c6ccccc6-c6ccccc65)cc4)c23)cc1]

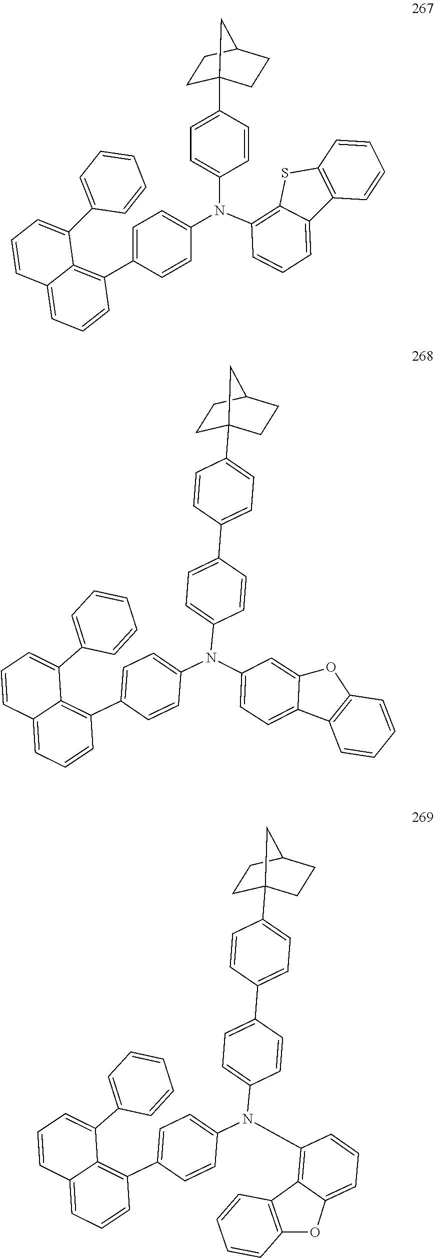 [SMILES: c1ccc(-c2cccc3cccc(-c4ccc(N(c5ccc(C67CCC(CC6)C7)cc5)c5cccc6c5sc5ccccc56)cc4)c23)cc1 ; c1ccc(-c2cccc3cccc(-c4ccc(N(c5ccc(-c6ccc(C78CCC(CC7)C8)cc6)cc5)c5ccc6c(c5)oc5ccccc56)cc4)c23)cc1 ; c1ccc(-c2cccc3cccc(-c4ccc(N(c5ccc(-c6ccc(C78CCC(CC7)C8)cc6)cc5)c5cccc6oc7ccccc7c56)cc4)c23)cc1]

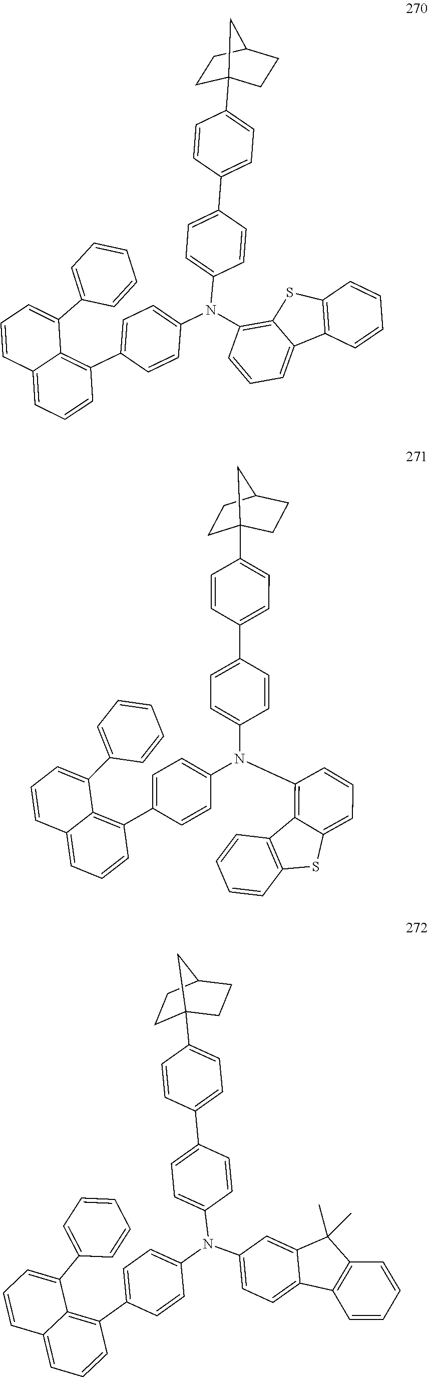 [SMILES: c1ccc(-c2cccc3cccc(-c4ccc(N(c5ccc(-c6ccc(C78CCC(CC7)C8)cc6)cc5)c5cccc6c5sc5ccccc56)cc4)c23)cc1 ; c1ccc(-c2cccc3cccc(-c4ccc(N(c5ccc(-c6ccc(C78CCC(CC7)C8)cc6)cc5)c5cccc6sc7ccccc7c56)cc4)c23)cc1 ; CC1(C)c2ccccc2-c2ccc(N(c3ccc(-c4ccc(C56CCC(CC5)C6)cc4)cc3)c3ccc(-c4cccc5cccc(-c6ccccc6)c45)cc3)cc21]

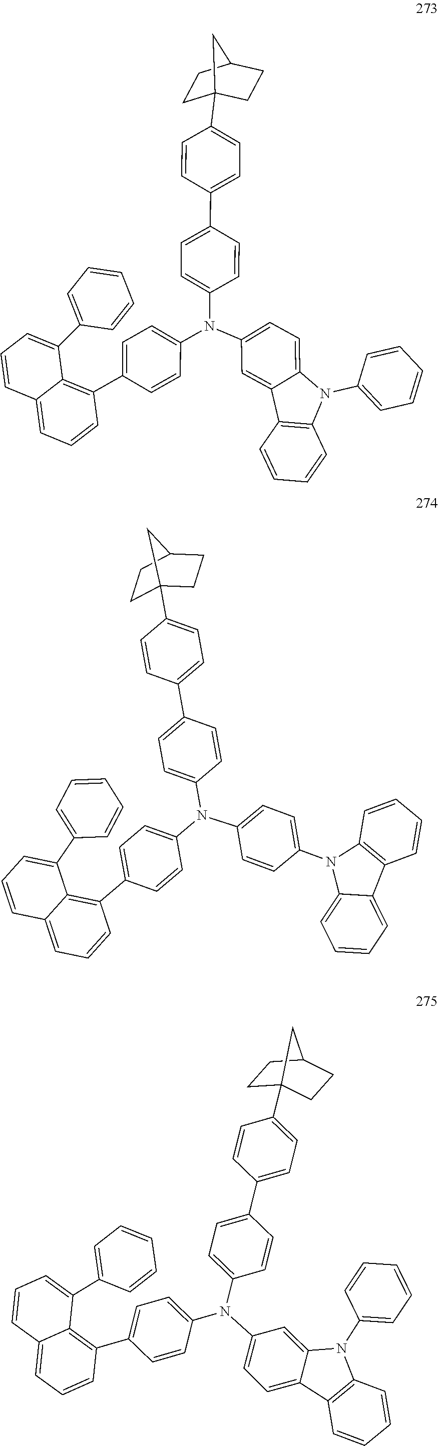 [SMILES: c1ccc(-c2cccc3cccc(-c4ccc(N(c5ccc(-c6ccc(C78CCC(CC7)C8)cc6)cc5)c5ccc6c(c5)c5ccccc5n6-c5ccccc5)cc4)c23)cc1 ; c1ccc(-c2cccc3cccc(-c4ccc(N(c5ccc(-c6ccc(C78CCC(CC7)C8)cc6)cc5)c5ccc(-n6c7ccccc7c7ccccc76)cc5)cc4)c23)cc1 ; c1ccc(-c2cccc3cccc(-c4ccc(N(c5ccc(-c6ccc(C78CCC(CC7)C8)cc6)cc5)c5ccc6c7ccccc7n(-c7ccccc7)c6c5)cc4)c23)cc1]

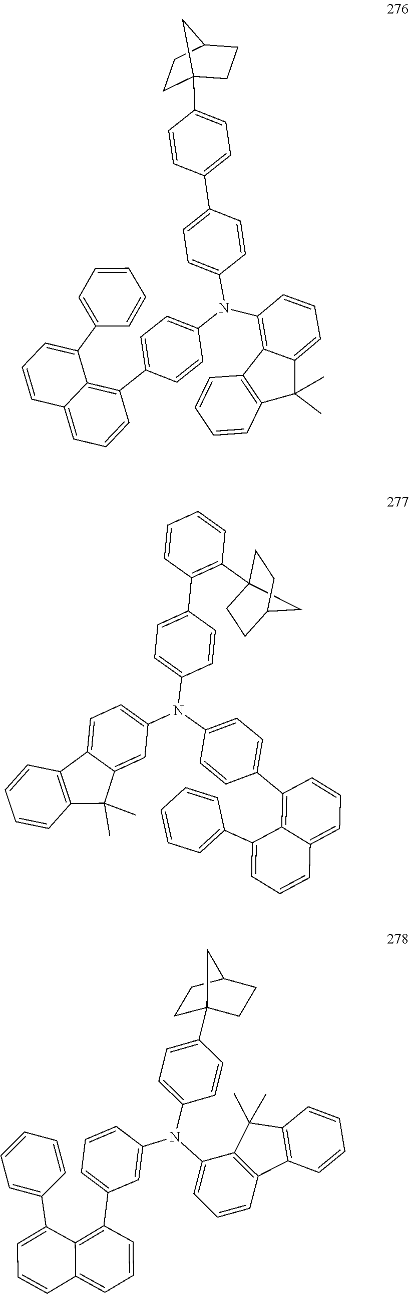 [SMILES: CC1(C)c2ccccc2-c2c(N(c3ccc(-c4ccc(C56CCC(CC5)C6)cc4)cc3)c3ccc(-c4cccc5cccc(-c6ccccc6)c45)cc3)cccc21 ; CC1(C)c2ccccc2-c2ccc(N(c3ccc(-c4ccccc4C45CCC(CC4)C5)cc3)c3ccc(-c4cccc5cccc(-c6ccccc6)c45)cc3)cc21 ; CC1(C)c2ccccc2-c2cccc(N(c3ccc(C45CCC(CC4)C5)cc3)c3cccc(-c4cccc5cccc(-c6ccccc6)c45)c3)c21]

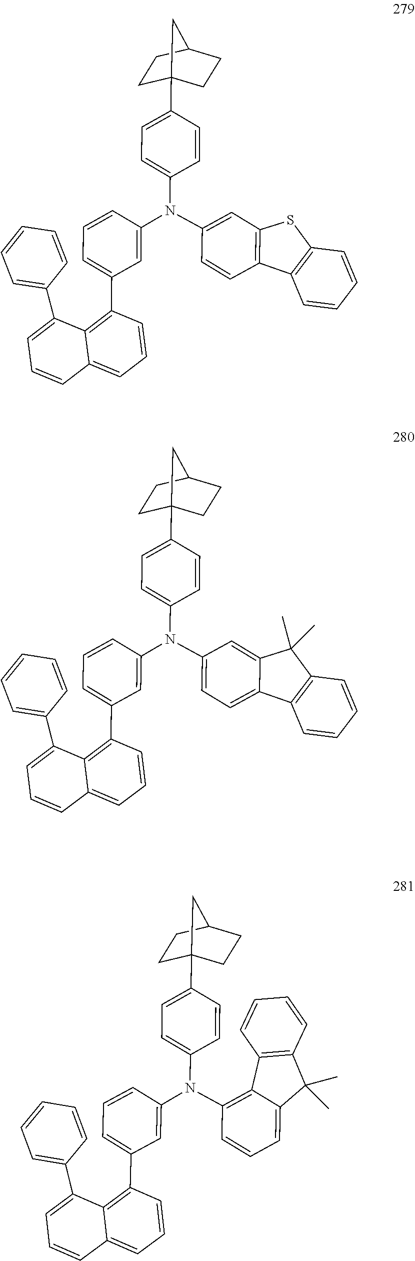 [SMILES: c1ccc(-c2cccc3cccc(-c4cccc(N(c5ccc(C67CCC(CC6)C7)cc5)c5ccc6c(c5)sc5ccccc56)c4)c23)cc1 ; CC1(C)c2ccccc2-c2ccc(N(c3ccc(C45CCC(CC4)C5)cc3)c3cccc(-c4cccc5cccc(-c6ccccc6)c45)c3)cc21 ; CC1(C)c2ccccc2-c2c(N(c3ccc(C45CCC(CC4)C5)cc3)c3cccc(-c4cccc5cccc(-c6ccccc6)c45)c3)cccc21]

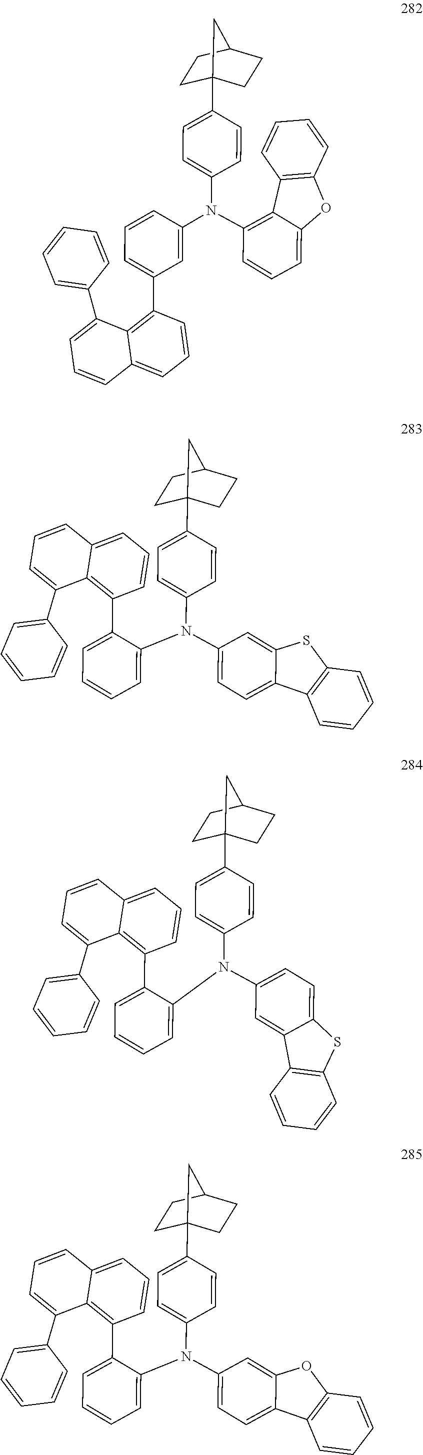 [SMILES: c1ccc(-c2cccc3cccc(-c4cccc(N(c5ccc(C67CCC(CC6)C7)cc5)c5cccc6oc7ccccc7c56)c4)c23)cc1 ; c1ccc(-c2cccc3cccc(-c4ccccc4N(c4ccc(C56CCC(CC5)C6)cc4)c4ccc5c(c4)sc4ccccc45)c23)cc1 ; c1ccc(-c2cccc3cccc(-c4ccccc4N(c4ccc(C56CCC(CC5)C6)cc4)c4ccc5sc6ccccc6c5c4)c23)cc1 ; c1ccc(-c2cccc3cccc(-c4ccccc4N(c4ccc(C56CCC(CC5)C6)cc4)c4ccc5c(c4)oc4ccccc45)c23)cc1]

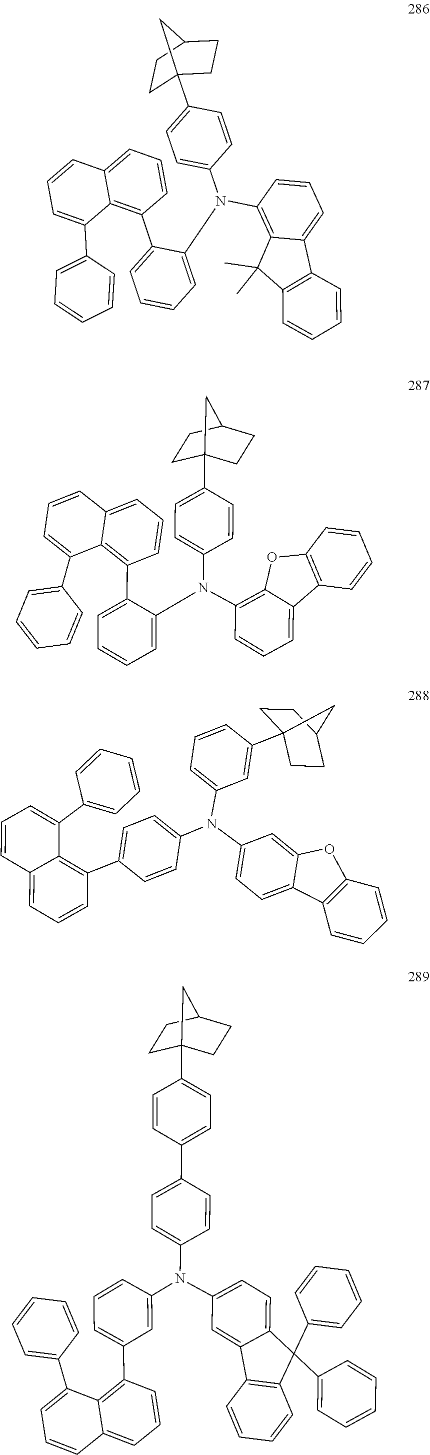 [SMILES: CC1(C)c2ccccc2-c2cccc(N(c3ccc(C45CCC(CC4)C5)cc3)c3ccccc3-c3cccc4cccc(-c5ccccc5)c34)c21 ; c1ccc(-c2cccc3cccc(-c4ccccc4N(c4ccc(C56CCC(CC5)C6)cc4)c4cccc5c4oc4ccccc45)c23)cc1 ; c1ccc(-c2cccc3cccc(-c4ccc(N(c5cccc(C67CCC(CC6)C7)c5)c5ccc6c(c5)oc5ccccc56)cc4)c23)cc1 ; c1ccc(-c2cccc3cccc(-c4cccc(N(c5ccc(-c6ccc(C78CCC(CC7)C8)cc6)cc5)c5ccc6c(c5)-c5ccccc5C6(c5ccccc5)c5ccccc5)c4)c23)cc1]

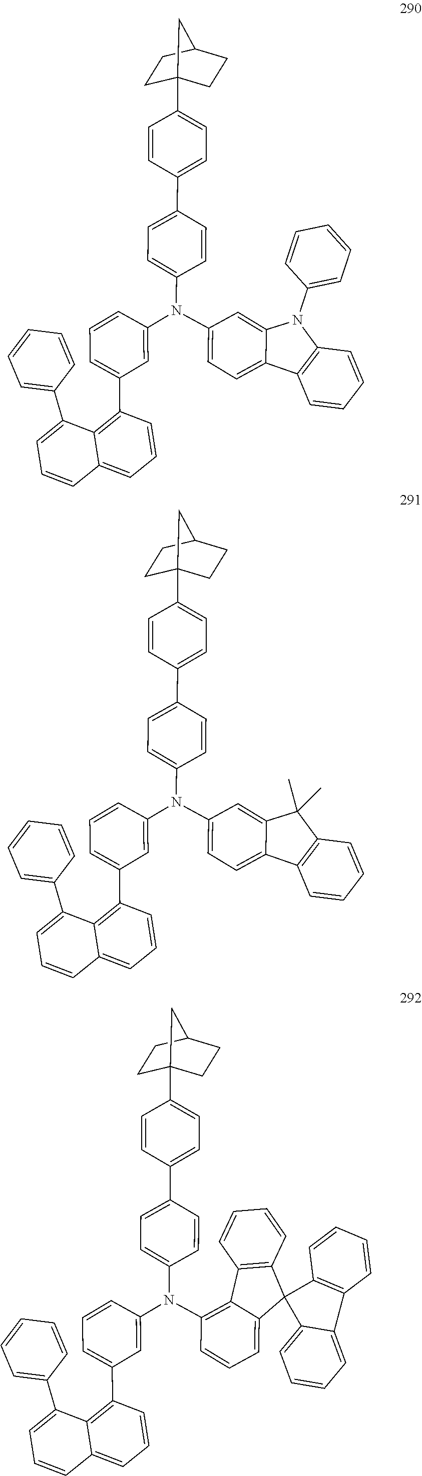 [SMILES: c1ccc(-c2cccc3cccc(-c4cccc(N(c5ccc(-c6ccc(C78CCC(CC7)C8)cc6)cc5)c5ccc6c7ccccc7n(-c7ccccc7)c6c5)c4)c23)cc1 ; CC1(C)c2ccccc2-c2ccc(N(c3ccc(-c4ccc(C56CCC(CC5)C6)cc4)cc3)c3cccc(-c4cccc5cccc(-c6ccccc6)c45)c3)cc21 ; c1ccc(-c2cccc3cccc(-c4cccc(N(c5ccc(-c6ccc(C78CCC(CC7)C8)cc6)cc5)c5cccc6c5-c5ccccc5C65c6ccccc6-c6ccccc65)c4)c23)cc1]

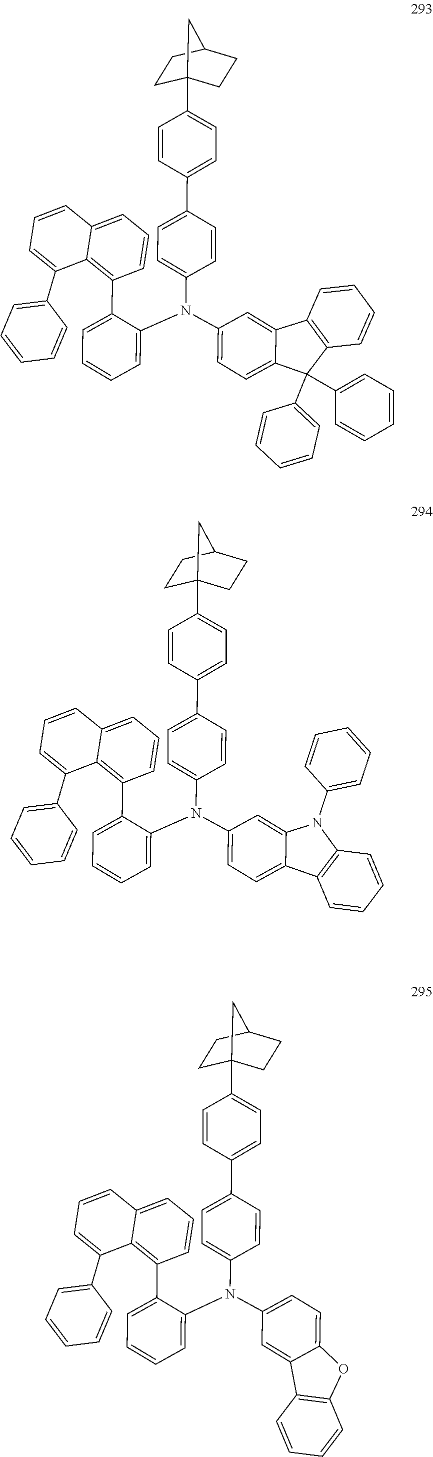 [SMILES: c1ccc(-c2cccc3cccc(-c4ccccc4N(c4ccc(-c5ccc(C67CCC(CC6)C7)cc5)cc4)c4ccc5c(c4)-c4ccccc4C5(c4ccccc4)c4ccccc4)c23)cc1 ; c1ccc(-c2cccc3cccc(-c4ccccc4N(c4ccc(-c5ccc(C67CCC(CC6)C7)cc5)cc4)c4ccc5c6ccccc6n(-c6ccccc6)c5c4)c23)cc1 ; c1ccc(-c2cccc3cccc(-c4ccccc4N(c4ccc(-c5ccc(C67CCC(CC6)C7)cc5)cc4)c4ccc5oc6ccccc6c5c4)c23)cc1]

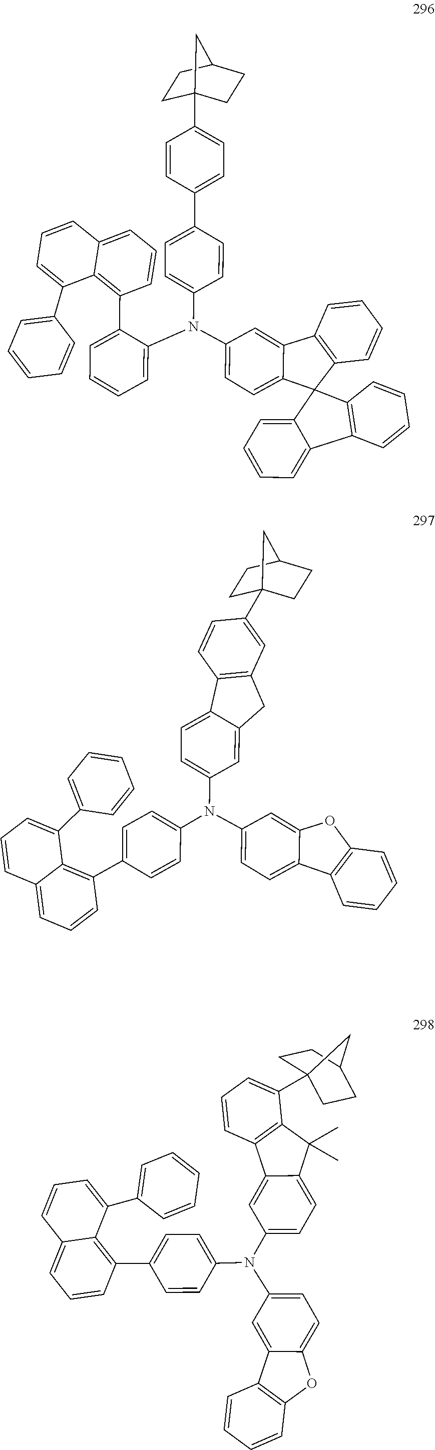 [SMILES: c1ccc(-c2cccc3cccc(-c4ccccc4N(c4ccc(-c5ccc(C67CCC(CC6)C7)cc5)cc4)c4ccc5c(c4)-c4ccccc4C54c5ccccc5-c5ccccc54)c23)cc1 ; c1ccc(-c2cccc3cccc(-c4ccc(N(c5ccc6c(c5)Cc5cc(C78CCC(CC7)C8)ccc5-6)c5ccc6c(c5)oc5ccccc56)cc4)c23)cc1 ; CC1(C)c2ccc(N(c3ccc(-c4cccc5cccc(-c6ccccc6)c45)cc3)c3ccc4oc5ccccc5c4c3)cc2-c2cccc(C34CCC(CC3)C4)c21]

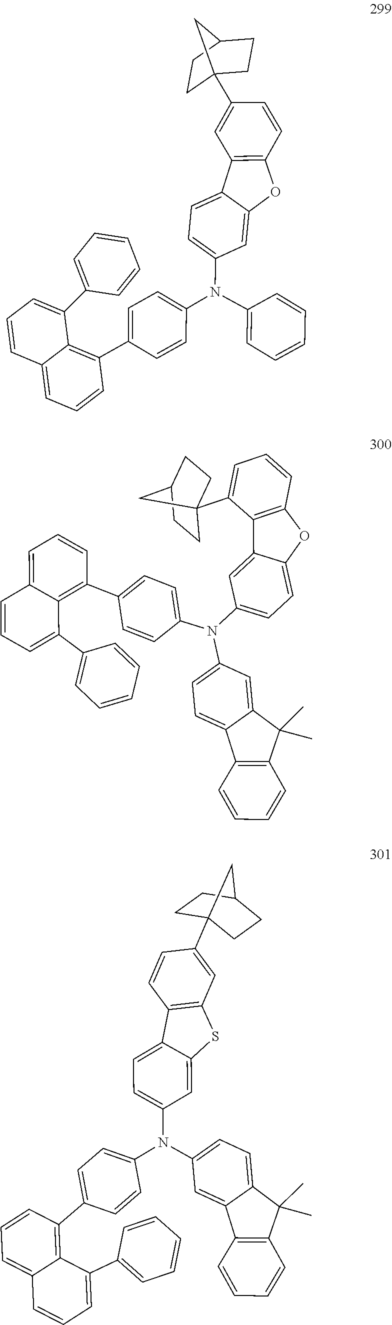

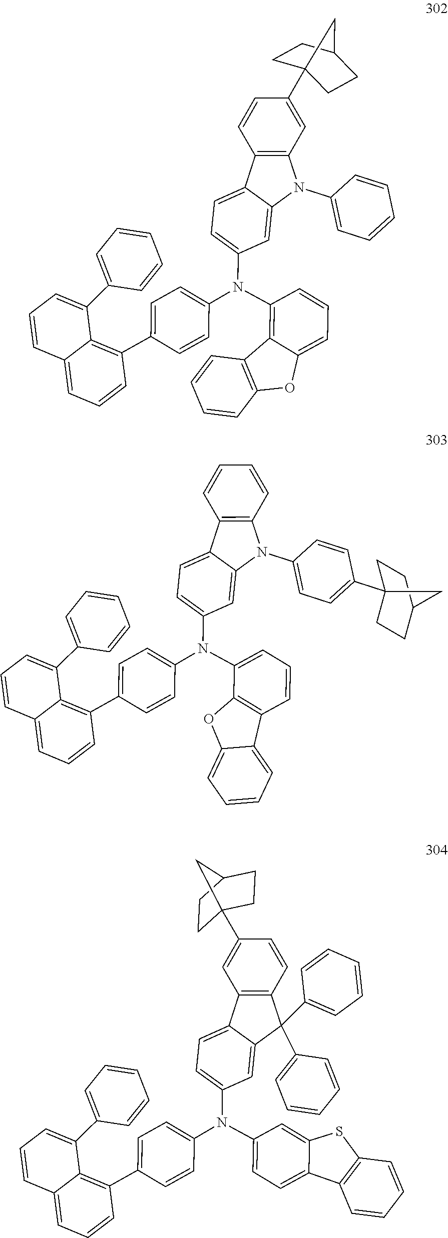 [SMILES: c1ccc(-c2cccc3cccc(-c4ccc(N(c5ccc6c7ccc(C89CCC(CC8)C9)cc7n(-c7ccccc7)c6c5)c5cccc6oc7ccccc7c56)cc4)c23)cc1 ; c1ccc(-c2cccc3cccc(-c4ccc(N(c5ccc6c7ccccc7n(-c7ccc(C89CCC(CC8)C9)cc7)c6c5)c5cccc6c5oc5ccccc56)cc4)c23)cc1 ; c1ccc(-c2cccc3cccc(-c4ccc(N(c5ccc6c(c5)C(c5ccccc5)(c5ccccc5)c5ccc(C78CCC(CC7)C8)cc5-6)c5ccc6c(c5)sc5ccccc56)cc4)c23)cc1]

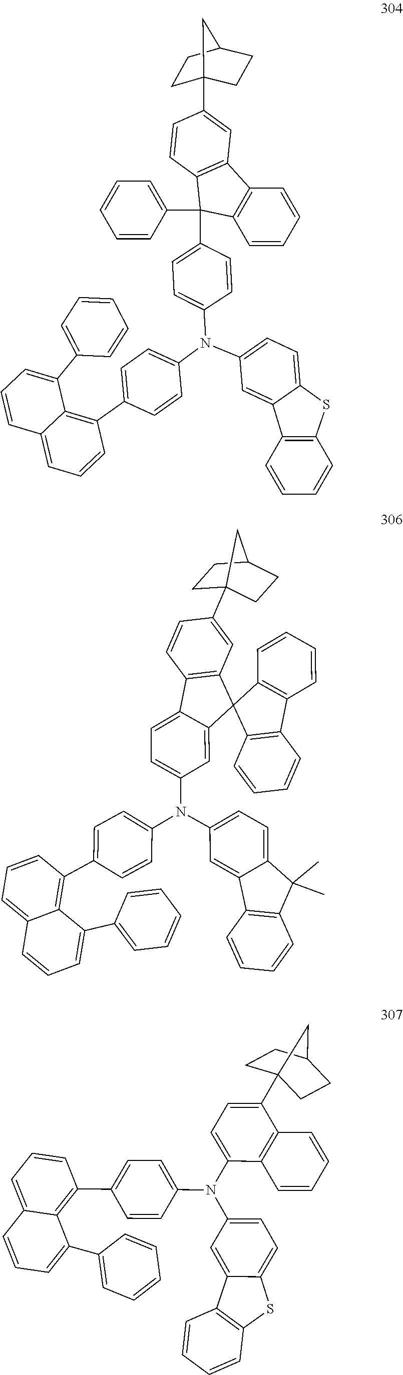

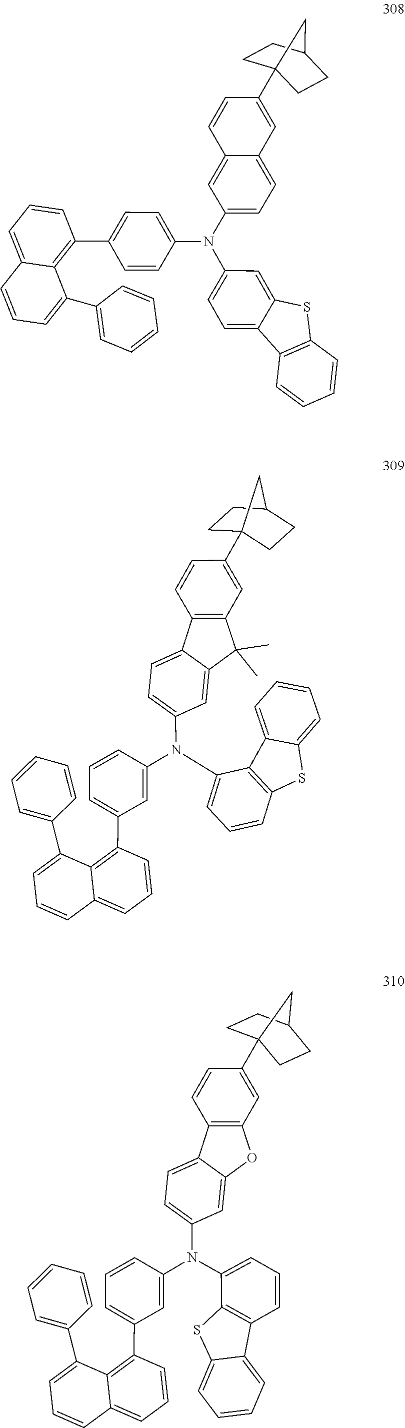

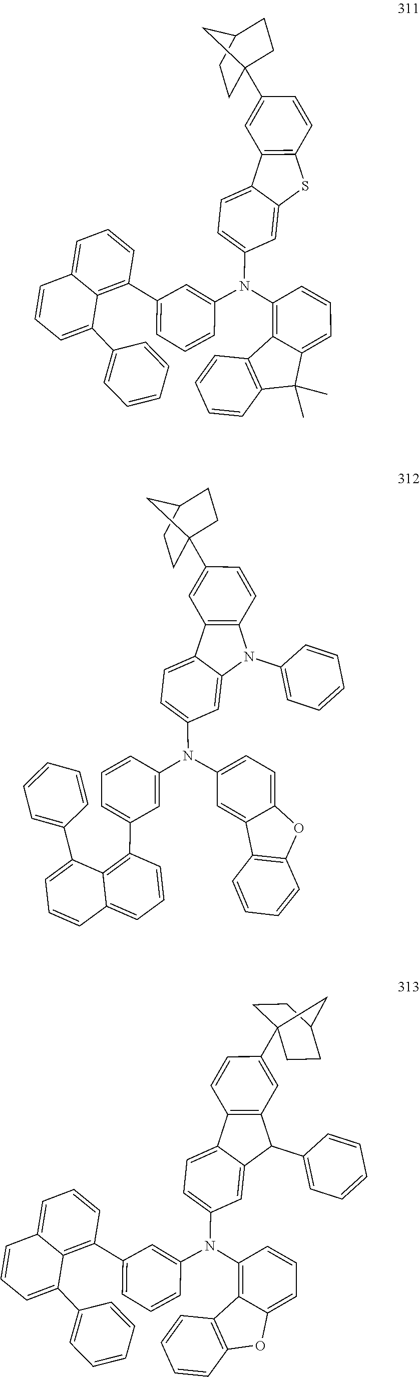

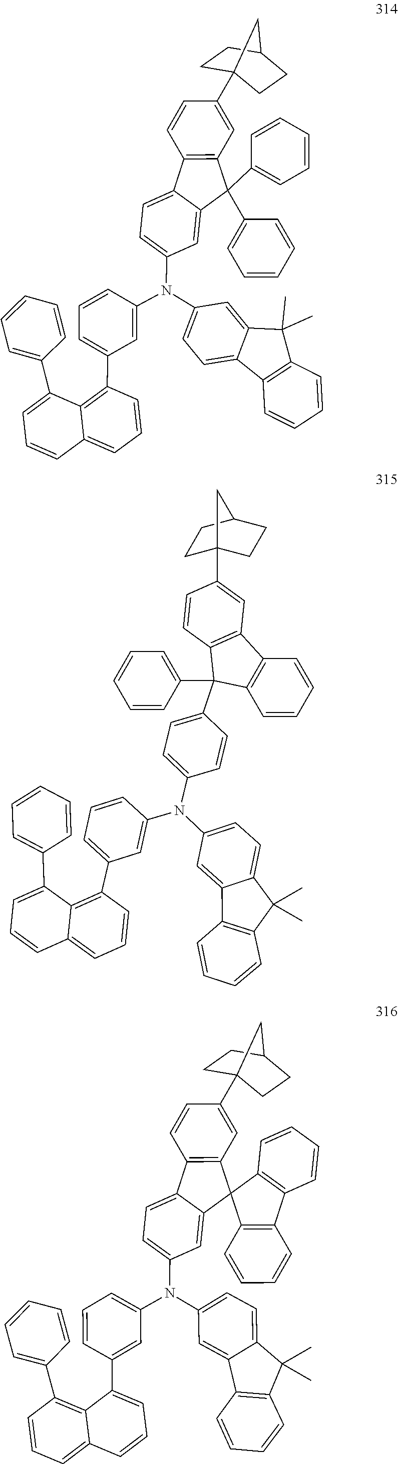

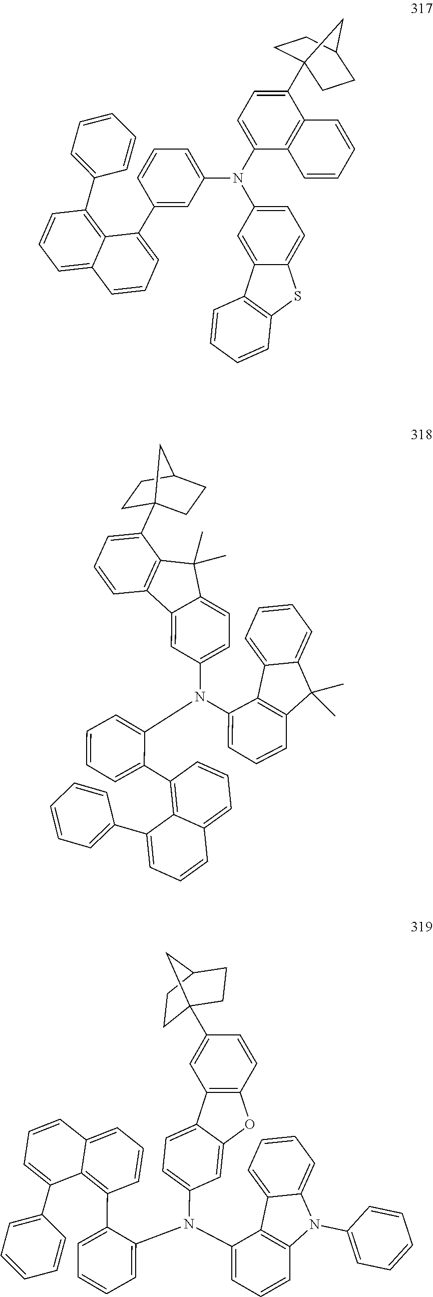 [SMILES: c1ccc(-c2cccc3cccc(-c4cccc(N(c5ccc6sc7ccccc7c6c5)c5ccc(C67CCC(CC6)C7)c6ccccc56)c4)c23)cc1 ; CC1(C)c2ccccc2-c2c(N(c3ccc4c(c3)-c3cccc(C56CCC(CC5)C6)c3C4(C)C)c3ccccc3-c3cccc4cccc(-c5ccccc5)c34)cccc21 ; c1ccc(-c2cccc3cccc(-c4ccccc4N(c4ccc5c(c4)oc4ccc(C67CCC(CC6)C7)cc45)c4cccc5c4c4ccccc4n5-c4ccccc4)c23)cc1]

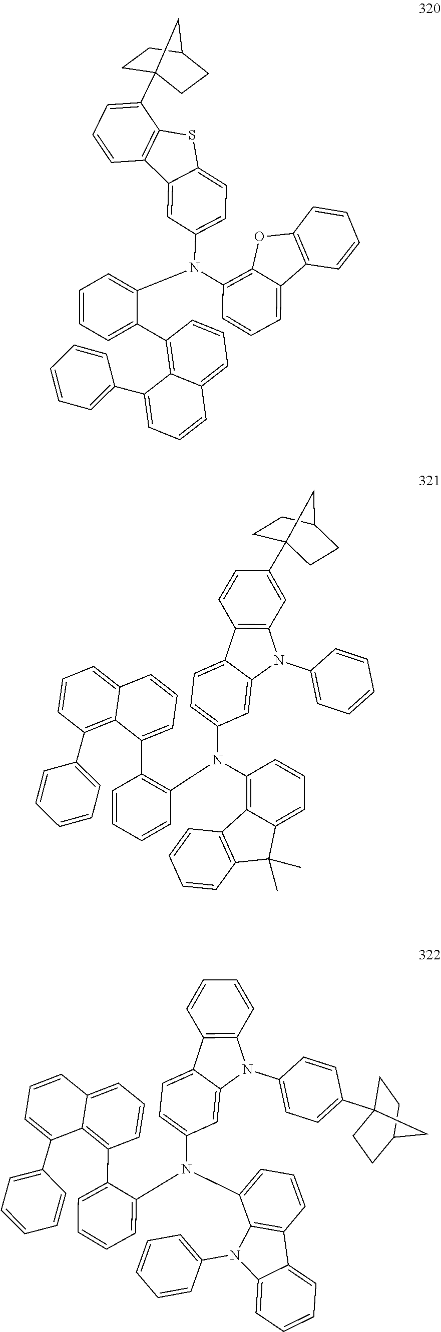 [SMILES: c1ccc(-c2cccc3cccc(-c4ccccc4N(c4ccc5sc6c(C78CCC(CC7)C8)cccc6c5c4)c4cccc5c4oc4ccccc45)c23)cc1 ; CC1(C)c2ccccc2-c2c(N(c3ccc4c5ccc(C67CCC(CC6)C7)cc5n(-c5ccccc5)c4c3)c3ccccc3-c3cccc4cccc(-c5ccccc5)c34)cccc21 ; c1ccc(-c2cccc3cccc(-c4ccccc4N(c4ccc5c6ccccc6n(-c6ccc(C78CCC(CC7)C8)cc6)c5c4)c4cccc5c6ccccc6n(-c6ccccc6)c45)c23)cc1]

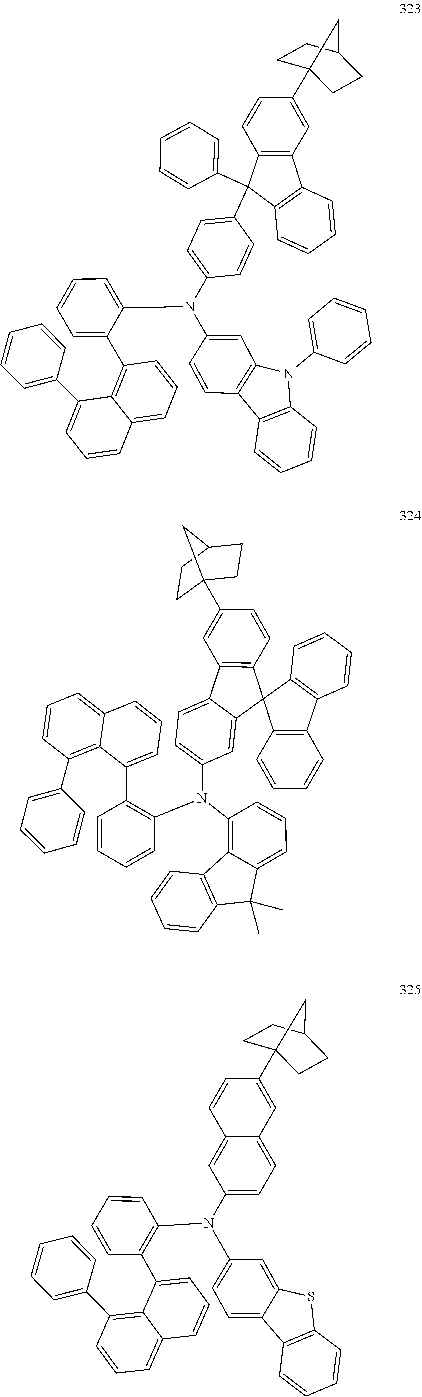 [SMILES: c1ccc(-c2cccc3cccc(-c4ccccc4N(c4ccc(C5(c6ccccc6)c6ccccc6-c6cc(C78CCC(CC7)C8)ccc65)cc4)c4ccc5c6ccccc6n(-c6ccccc6)c5c4)c23)cc1 ; CC1(C)c2ccccc2-c2c(N(c3ccc4c(c3)C3(c5ccccc5-c5ccccc53)c3ccc(C56CCC(CC5)C6)cc3-4)c3ccccc3-c3cccc4cccc(-c5ccccc5)c34)cccc21 ; c1ccc(-c2cccc3cccc(-c4ccccc4N(c4ccc5cc(C67CCC(CC6)C7)ccc5c4)c4ccc5c(c4)sc4ccccc45)c23)cc1]

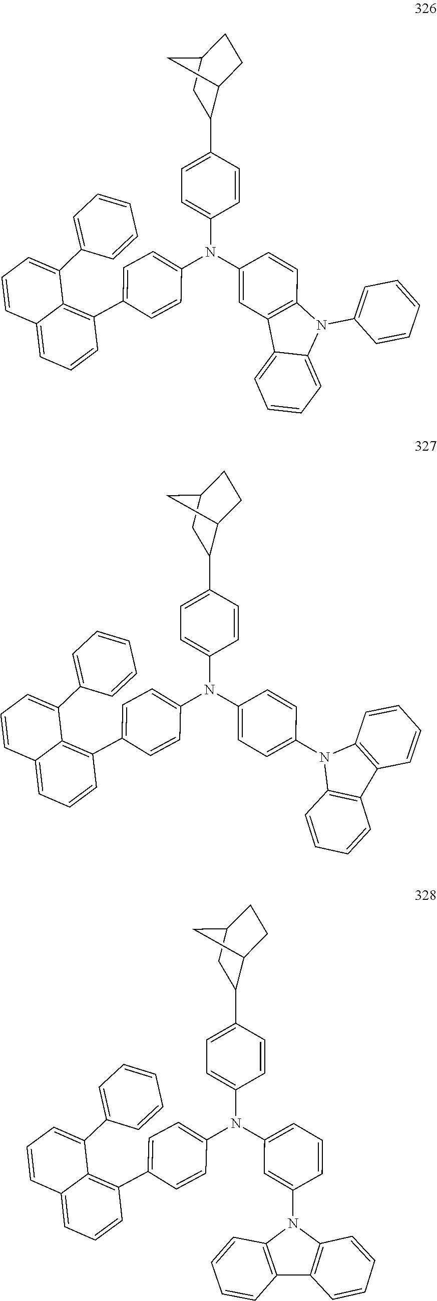 [SMILES: c1ccc(-c2cccc3cccc(-c4ccc(N(c5ccc(C6CC7CCC6C7)cc5)c5ccc6c(c5)c5ccccc5n6-c5ccccc5)cc4)c23)cc1 ; c1ccc(-c2cccc3cccc(-c4ccc(N(c5ccc(C6CC7CCC6C7)cc5)c5ccc(-n6c7ccccc7c7ccccc76)cc5)cc4)c23)cc1 ; c1ccc(-c2cccc3cccc(-c4ccc(N(c5ccc(C6CC7CCC6C7)cc5)c5cccc(-n6c7ccccc7c7ccccc76)c5)cc4)c23)cc1]

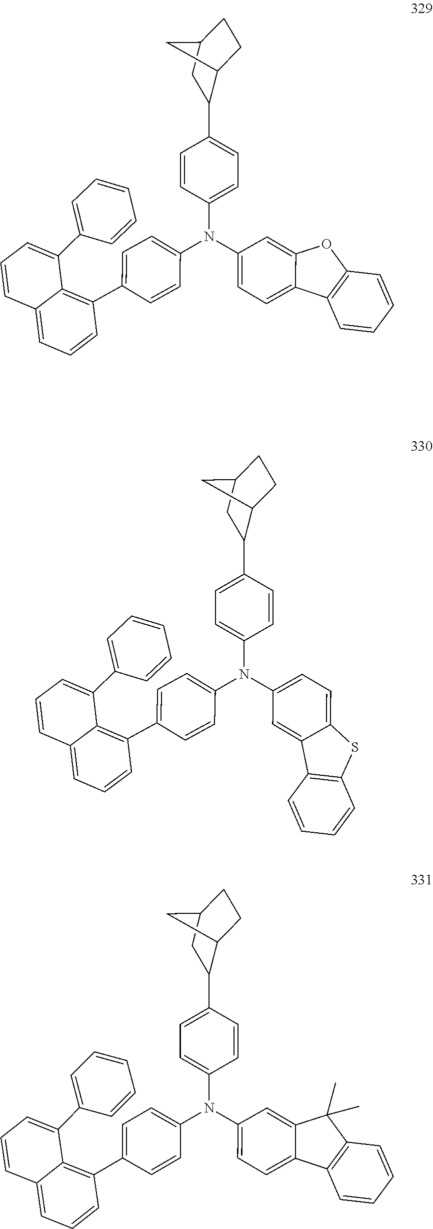 [SMILES: c1ccc(-c2cccc3cccc(-c4ccc(N(c5ccc(C6CC7CCC6C7)cc5)c5ccc6c(c5)oc5ccccc56)cc4)c23)cc1 ; c1ccc(-c2cccc3cccc(-c4ccc(N(c5ccc(C6CC7CCC6C7)cc5)c5ccc6sc7ccccc7c6c5)cc4)c23)cc1 ; CC1(C)c2ccccc2-c2ccc(N(c3ccc(-c4cccc5cccc(-c6ccccc6)c45)cc3)c3ccc(C4CC5CCC4C5)cc3)cc21]

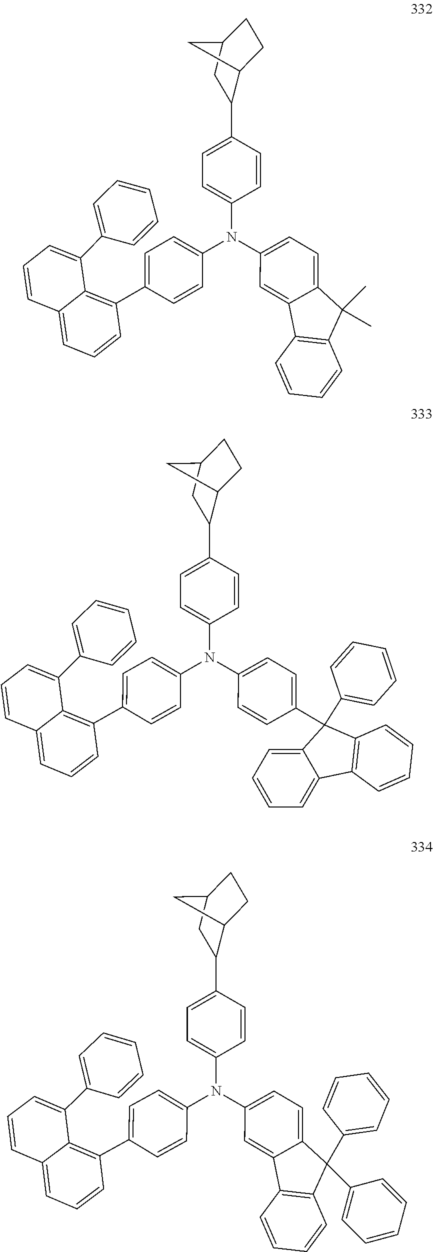 [SMILES: CC1(C)c2ccccc2-c2cc(N(c3ccc(-c4cccc5cccc(-c6ccccc6)c45)cc3)c3ccc(C4CC5CCC4C5)cc3)ccc21 ; c1ccc(-c2cccc3cccc(-c4ccc(N(c5ccc(C6CC7CCC6C7)cc5)c5ccc(C6(c7ccccc7)c7ccccc7-c7ccccc76)cc5)cc4)c23)cc1 ; c1ccc(-c2cccc3cccc(-c4ccc(N(c5ccc(C6CC7CCC6C7)cc5)c5ccc6c(c5)-c5ccccc5C6(c5ccccc5)c5ccccc5)cc4)c23)cc1]

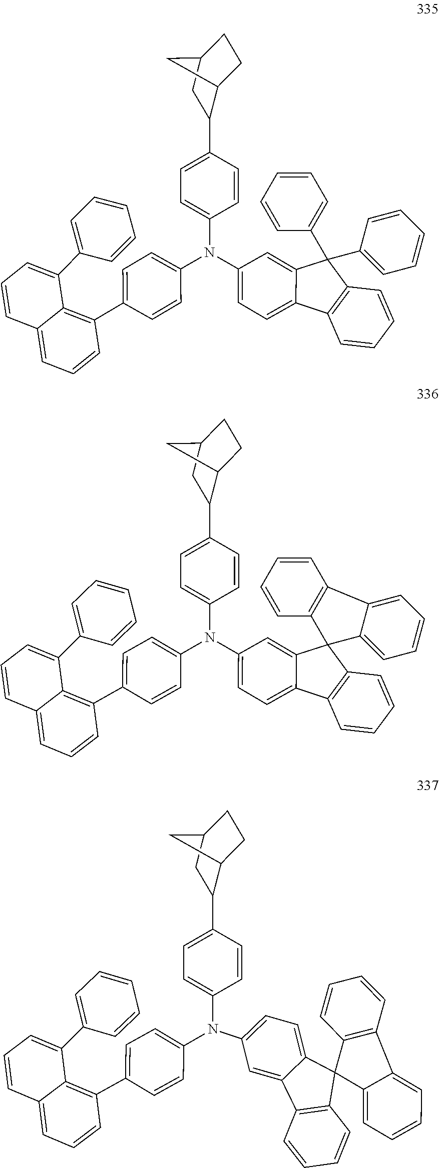 [SMILES: c1ccc(-c2cccc3cccc(-c4ccc(N(c5ccc(C6CC7CCC6C7)cc5)c5ccc6c(c5)C(c5ccccc5)(c5ccccc5)c5ccccc5-6)cc4)c23)cc1 ; c1ccc(-c2cccc3cccc(-c4ccc(N(c5ccc(C6CC7CCC6C7)cc5)c5ccc6c(c5)C5(c7ccccc7-c7ccccc75)c5ccccc5-6)cc4)c23)cc1 ; c1ccc(-c2cccc3cccc(-c4ccc(N(c5ccc(C6CC7CCC6C7)cc5)c5ccc6c(c5)-c5ccccc5C65c6ccccc6-c6ccccc65)cc4)c23)cc1]

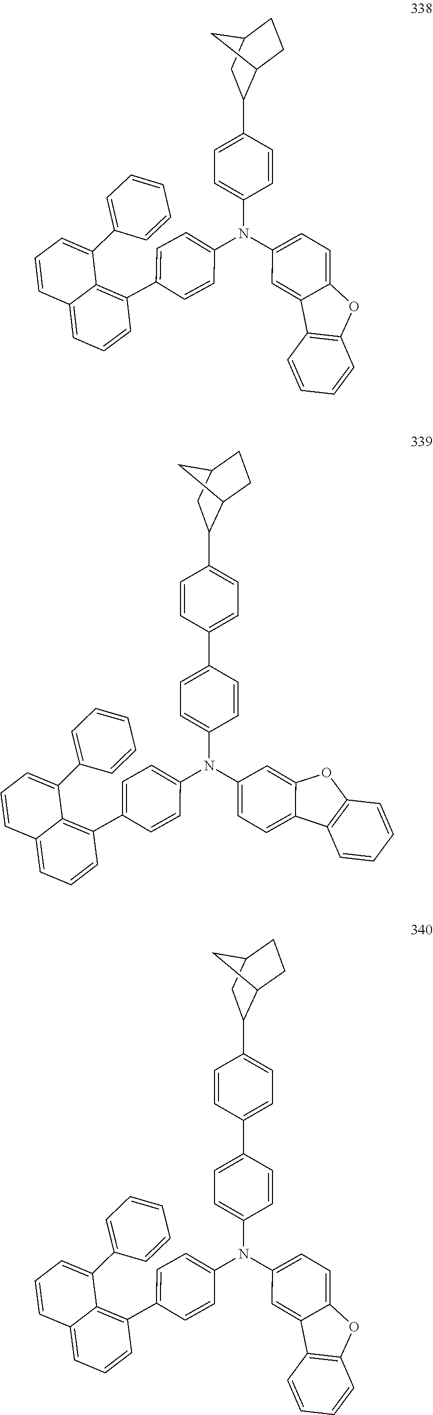 [SMILES: c1ccc(-c2cccc3cccc(-c4ccc(N(c5ccc(C6CC7CCC6C7)cc5)c5ccc6oc7ccccc7c6c5)cc4)c23)cc1 ; c1ccc(-c2cccc3cccc(-c4ccc(N(c5ccc(-c6ccc(C7CC8CCC7C8)cc6)cc5)c5ccc6c(c5)oc5ccccc56)cc4)c23)cc1 ; c1ccc(-c2cccc3cccc(-c4ccc(N(c5ccc(-c6ccc(C7CC8CCC7C8)cc6)cc5)c5ccc6oc7ccccc7c6c5)cc4)c23)cc1]

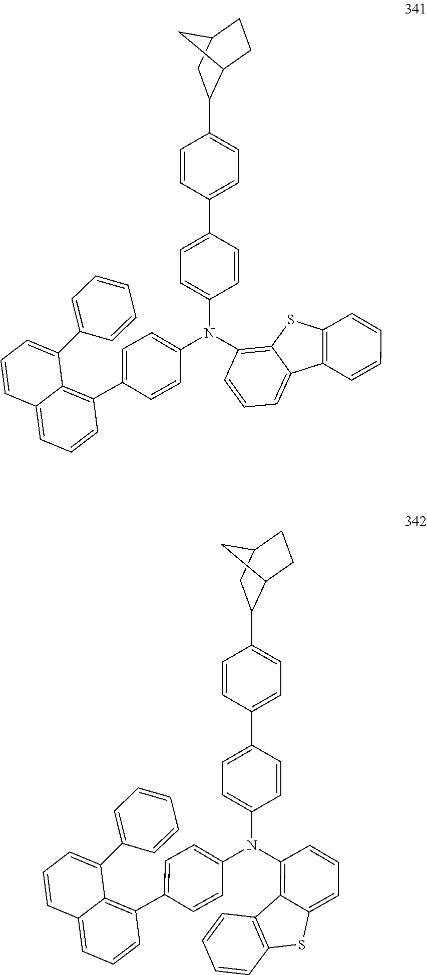 [SMILES: c1ccc(-c2cccc3cccc(-c4ccc(N(c5ccc(-c6ccc(C7CC8CCC7C8)cc6)cc5)c5cccc6c5sc5ccccc56)cc4)c23)cc1 ; c1ccc(-c2cccc3cccc(-c4ccc(N(c5ccc(-c6ccc(C7CC8CCC7C8)cc6)cc5)c5cccc6sc7ccccc7c56)cc4)c23)cc1]

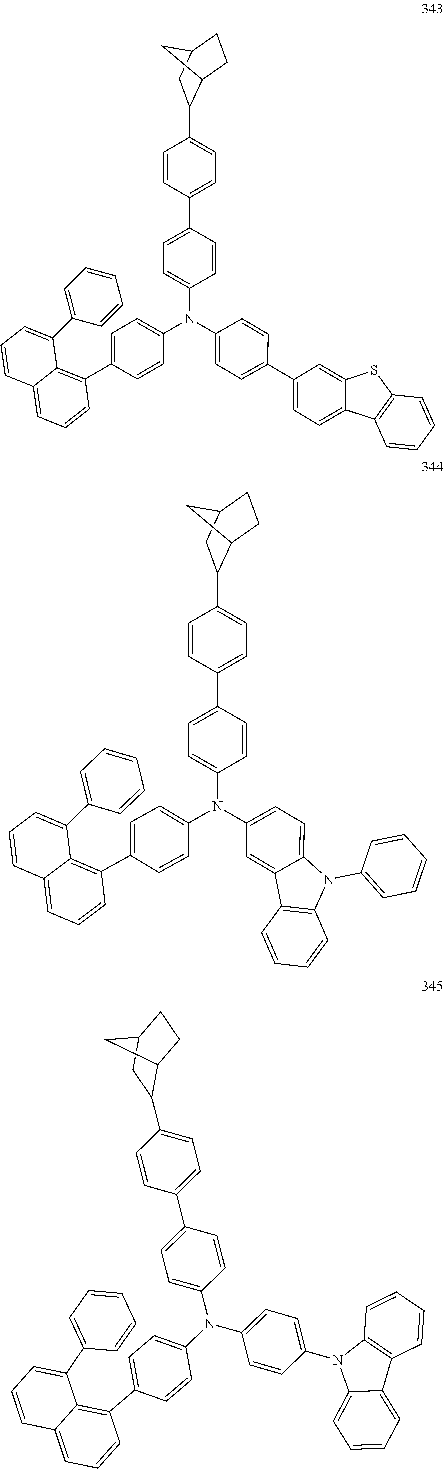 [SMILES: c1ccc(-c2cccc3cccc(-c4ccc(N(c5ccc(-c6ccc(C7CC8CCC7C8)cc6)cc5)c5ccc(-c6ccc7c(c6)sc6ccccc67)cc5)cc4)c23)cc1 ; c1ccc(-c2cccc3cccc(-c4ccc(N(c5ccc(-c6ccc(C7CC8CCC7C8)cc6)cc5)c5ccc6c(c5)c5ccccc5n6-c5ccccc5)cc4)c23)cc1 ; c1ccc(-c2cccc3cccc(-c4ccc(N(c5ccc(-c6ccc(C7CC8CCC7C8)cc6)cc5)c5ccc(-n6c7ccccc7c7ccccc76)cc5)cc4)c23)cc1]

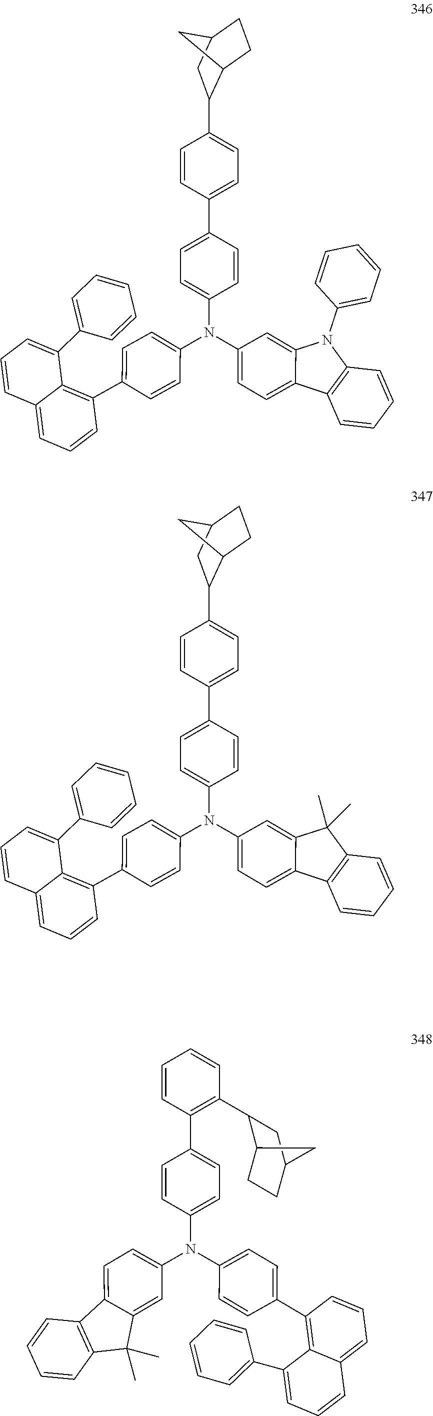 [SMILES: c1ccc(-c2cccc3cccc(-c4ccc(N(c5ccc(-c6ccc(C7CC8CCC7C8)cc6)cc5)c5ccc6c7ccccc7n(-c7ccccc7)c6c5)cc4)c23)cc1 ; CC1(C)c2ccccc2-c2ccc(N(c3ccc(-c4ccc(C5CC6CCC5C6)cc4)cc3)c3ccc(-c4cccc5cccc(-c6ccccc6)c45)cc3)cc21 ; CC1(C)c2ccccc2-c2ccc(N(c3ccc(-c4ccccc4C4CC5CCC4C5)cc3)c3ccc(-c4cccc5cccc(-c6ccccc6)c45)cc3)cc21]

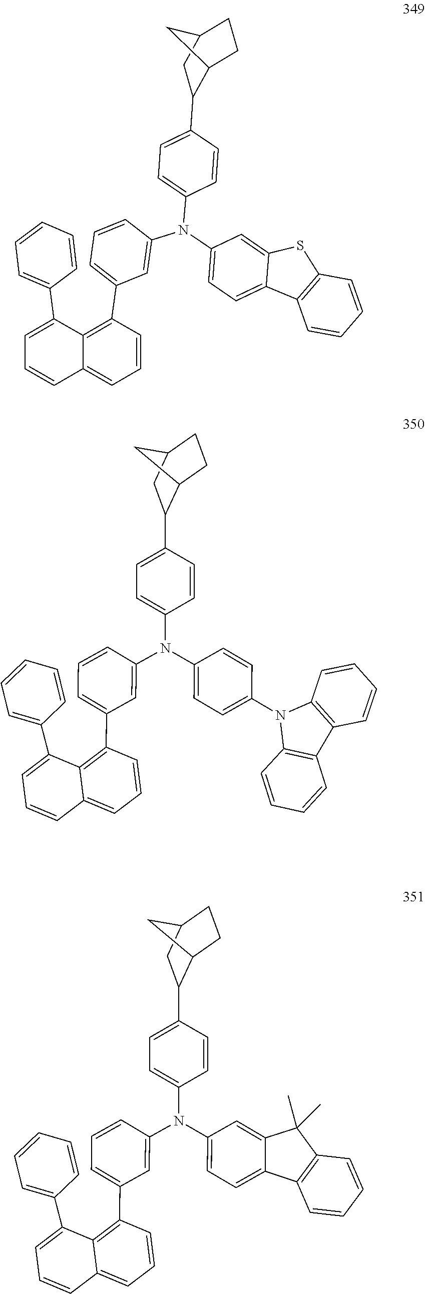 [SMILES: c1ccc(-c2cccc3cccc(-c4cccc(N(c5ccc(C6CC7CCC6C7)cc5)c5ccc6c(c5)sc5ccccc56)c4)c23)cc1 ; c1ccc(-c2cccc3cccc(-c4cccc(N(c5ccc(C6CC7CCC6C7)cc5)c5ccc(-n6c7ccccc7c7ccccc76)cc5)c4)c23)cc1 ; CC1(C)c2ccccc2-c2ccc(N(c3ccc(C4CC5CCC4C5)cc3)c3cccc(-c4cccc5cccc(-c6ccccc6)c45)c3)cc21]

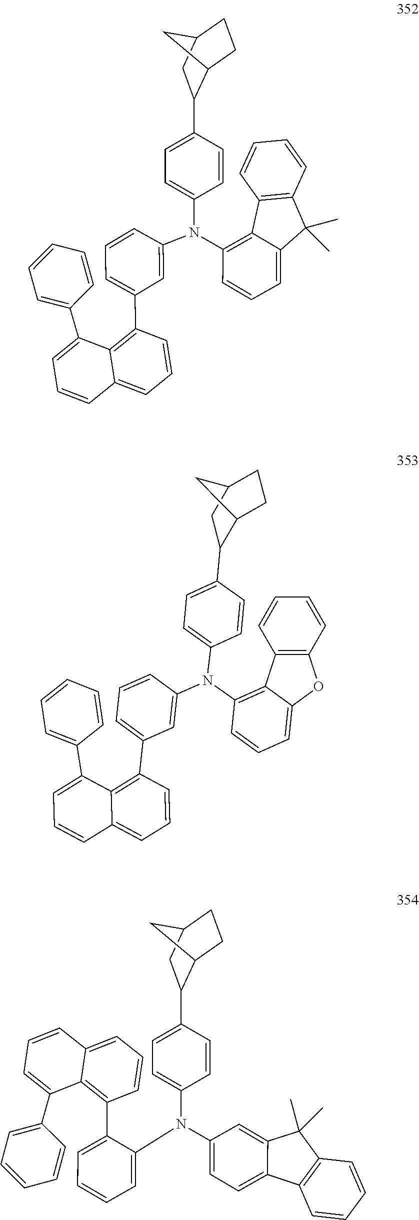 [SMILES: CC1(C)c2ccccc2-c2c(N(c3ccc(C4CC5CCC4C5)cc3)c3cccc(-c4cccc5cccc(-c6ccccc6)c45)c3)cccc21 ; c1ccc(-c2cccc3cccc(-c4cccc(N(c5ccc(C6CC7CCC6C7)cc5)c5cccc6oc7ccccc7c56)c4)c23)cc1 ; CC1(C)c2ccccc2-c2ccc(N(c3ccc(C4CC5CCC4C5)cc3)c3ccccc3-c3cccc4cccc(-c5ccccc5)c34)cc21]

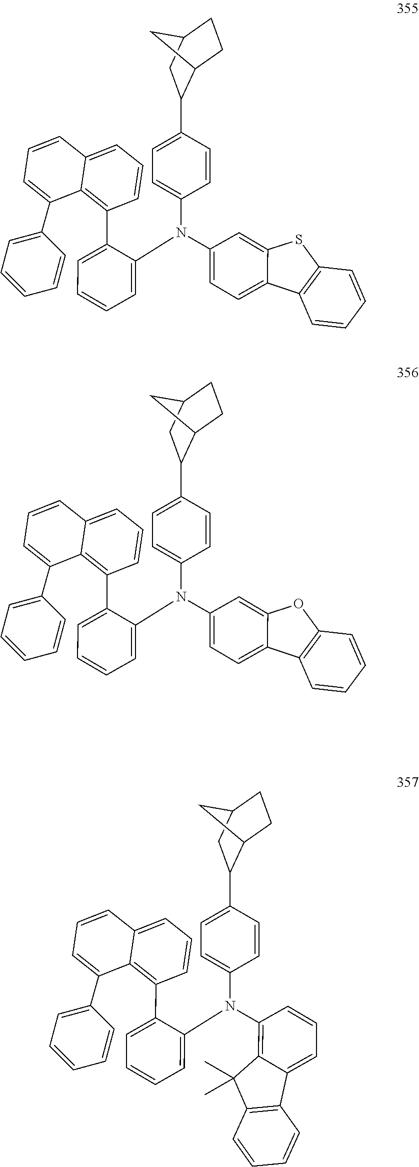 [SMILES: c1ccc(-c2cccc3cccc(-c4ccccc4N(c4ccc(C5CC6CCC5C6)cc4)c4ccc5c(c4)sc4ccccc45)c23)cc1 ; c1ccc(-c2cccc3cccc(-c4ccccc4N(c4ccc(C5CC6CCC5C6)cc4)c4ccc5c(c4)oc4ccccc45)c23)cc1 ; CC1(C)c2ccccc2-c2cccc(N(c3ccc(C4CC5CCC4C5)cc3)c3ccccc3-c3cccc4cccc(-c5ccccc5)c34)c21]

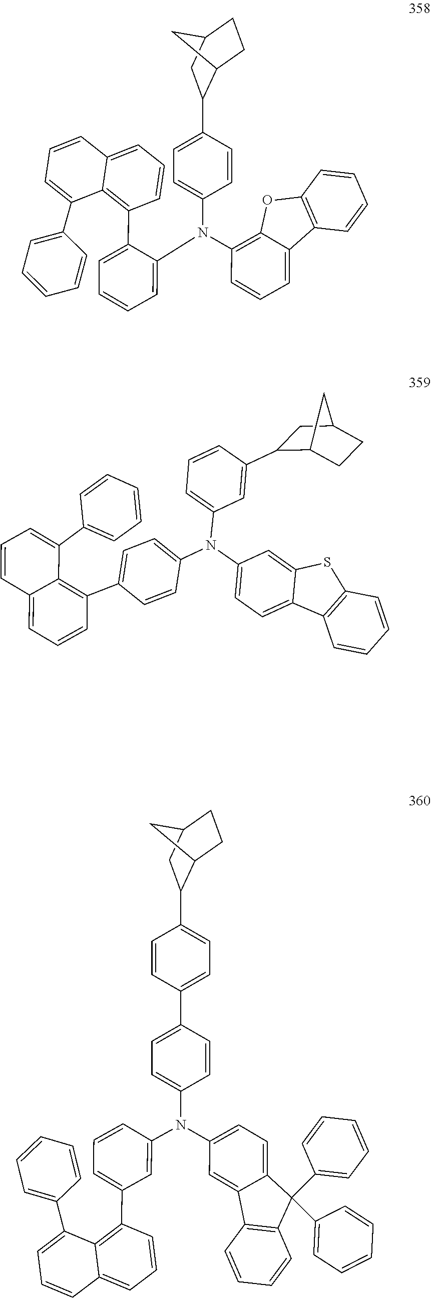 [SMILES: c1ccc(-c2cccc3cccc(-c4ccccc4N(c4ccc(C5CC6CCC5C6)cc4)c4cccc5c4oc4ccccc45)c23)cc1 ; c1ccc(-c2cccc3cccc(-c4ccc(N(c5cccc(C6CC7CCC6C7)c5)c5ccc6c(c5)sc5ccccc56)cc4)c23)cc1 ; c1ccc(-c2cccc3cccc(-c4cccc(N(c5ccc(-c6ccc(C7CC8CCC7C8)cc6)cc5)c5ccc6c(c5)-c5ccccc5C6(c5ccccc5)c5ccccc5)c4)c23)cc1]

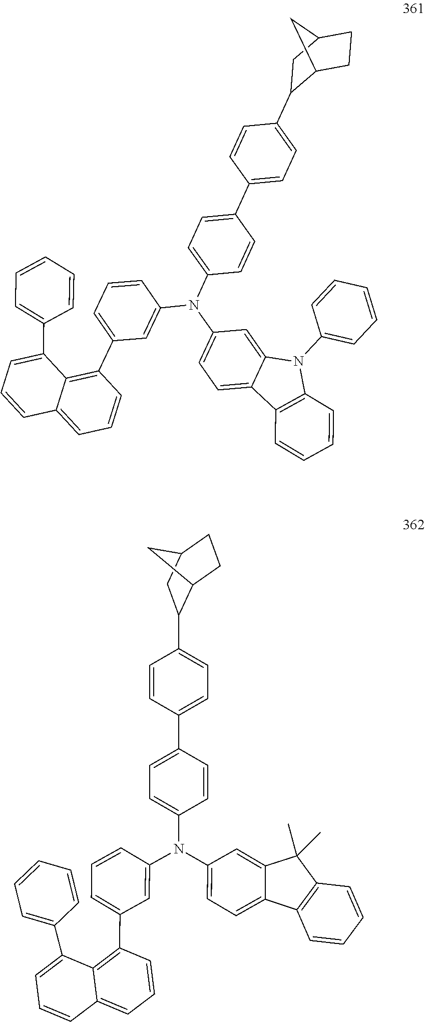 [SMILES: c1ccc(-c2cccc3cccc(-c4cccc(N(c5ccc(-c6ccc(C7CC8CCC7C8)cc6)cc5)c5ccc6c7ccccc7n(-c7ccccc7)c6c5)c4)c23)cc1 ; CC1(C)c2ccccc2-c2ccc(N(c3ccc(-c4ccc(C5CC6CCC5C6)cc4)cc3)c3cccc(-c4cccc5cccc(-c6ccccc6)c45)c3)cc21]

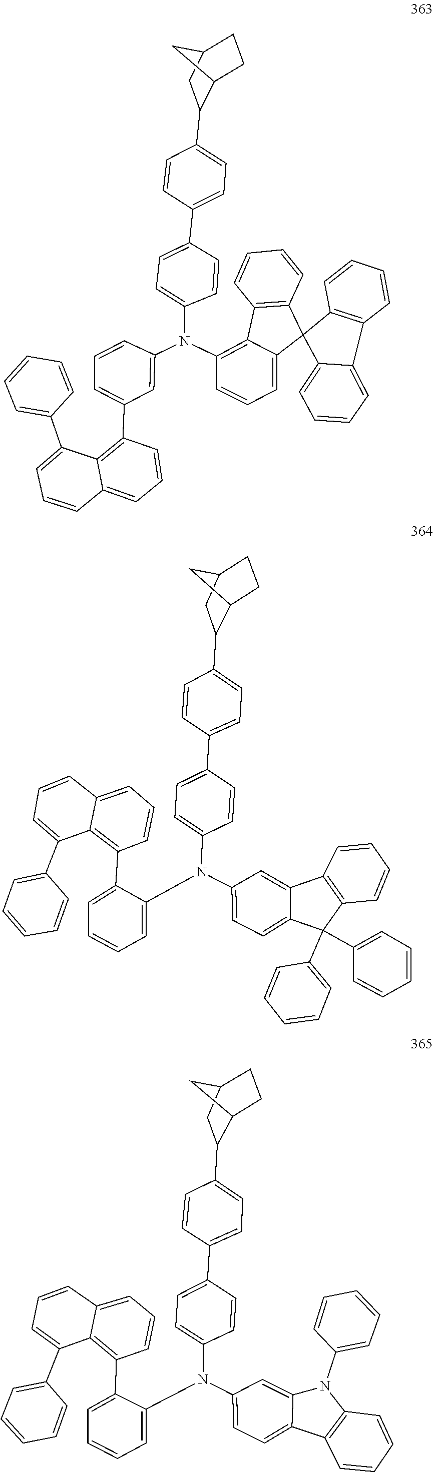 [SMILES: c1ccc(-c2cccc3cccc(-c4cccc(N(c5ccc(-c6ccc(C7CC8CCC7C8)cc6)cc5)c5cccc6c5-c5ccccc5C65c6ccccc6-c6ccccc65)c4)c23)cc1 ; c1ccc(-c2cccc3cccc(-c4ccccc4N(c4ccc(-c5ccc(C6CC7CCC6C7)cc5)cc4)c4ccc5c(c4)-c4ccccc4C5(c4ccccc4)c4ccccc4)c23)cc1 ; c1ccc(-c2cccc3cccc(-c4ccccc4N(c4ccc(-c5ccc(C6CC7CCC6C7)cc5)cc4)c4ccc5c6ccccc6n(-c6ccccc6)c5c4)c23)cc1]

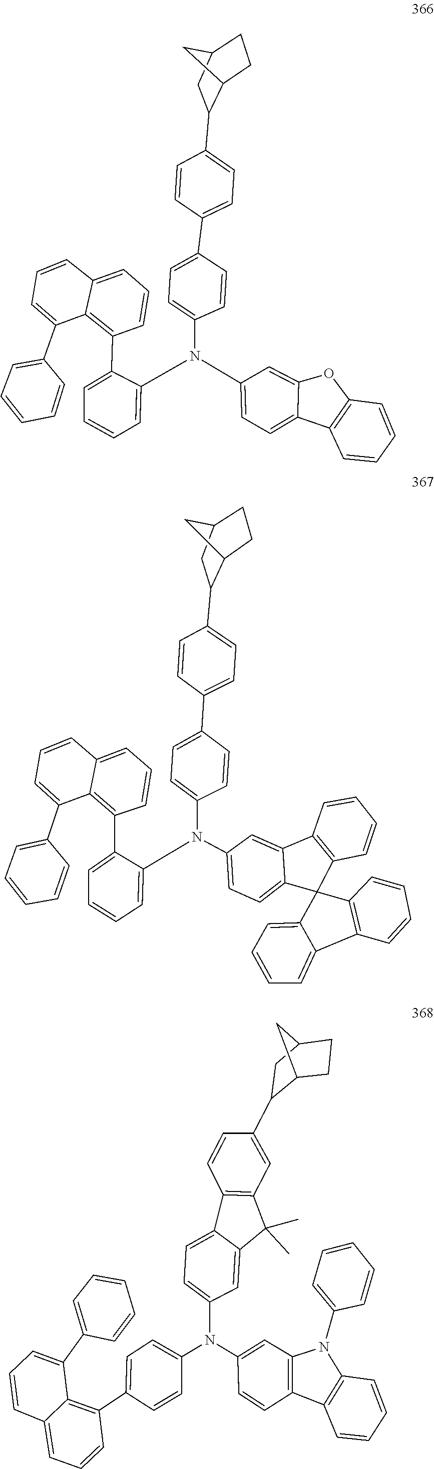

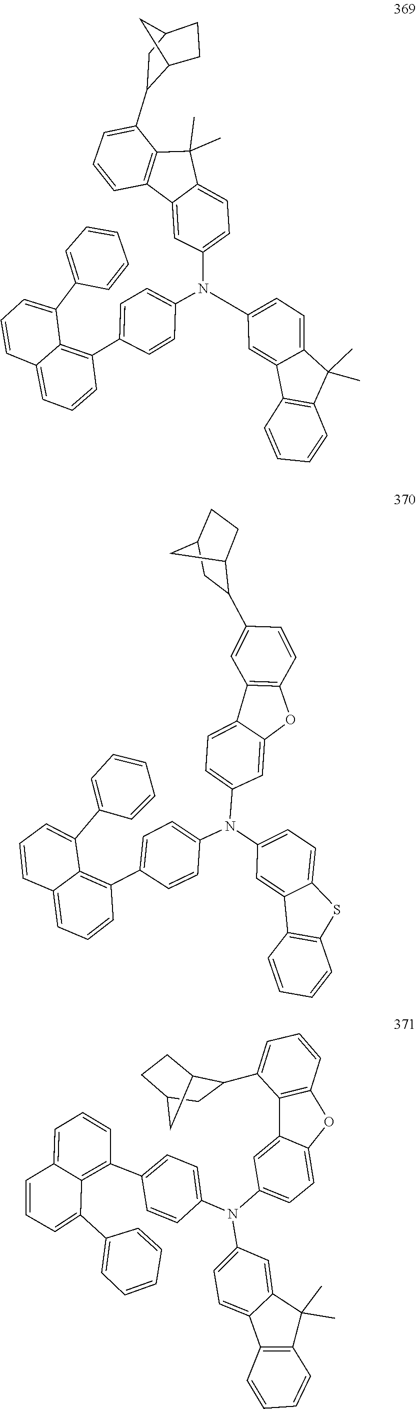

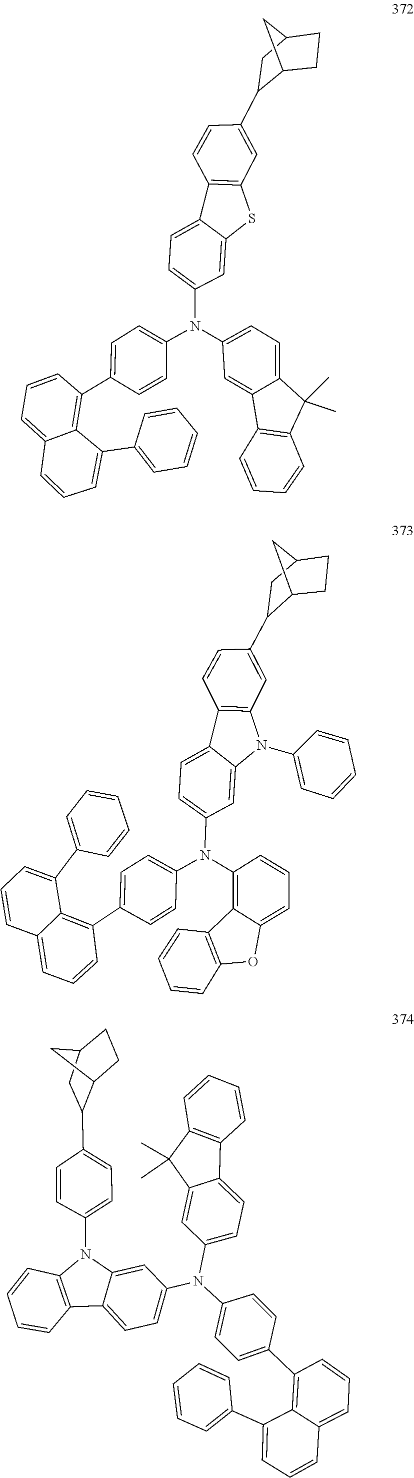 [SMILES: CC1(C)c2ccccc2-c2cc(N(c3ccc(-c4cccc5cccc(-c6ccccc6)c45)cc3)c3ccc4c(c3)sc3cc(C5CC6CCC5C6)ccc34)ccc21 ; c1ccc(-c2cccc3cccc(-c4ccc(N(c5ccc6c7ccc(C8CC9CCC8C9)cc7n(-c7ccccc7)c6c5)c5cccc6oc7ccccc7c56)cc4)c23)cc1 ; CC1(C)c2ccccc2-c2ccc(N(c3ccc(-c4cccc5cccc(-c6ccccc6)c45)cc3)c3ccc4c5ccccc5n(-c5ccc(C6CC7CCC6C7)cc5)c4c3)cc21]

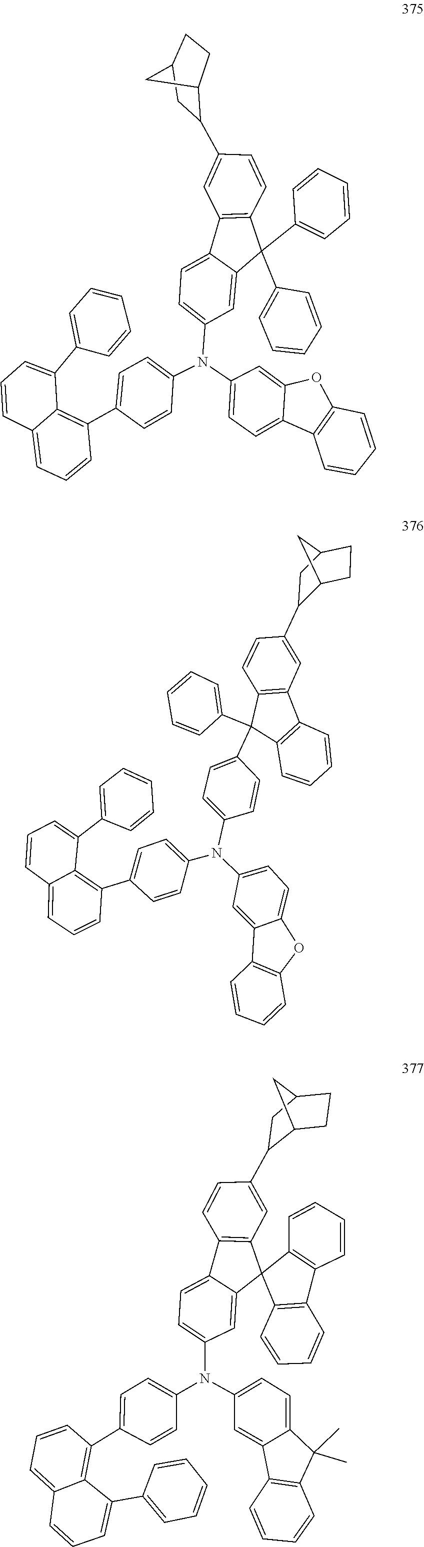 [SMILES: c1ccc(-c2cccc3cccc(-c4ccc(N(c5ccc6c(c5)C(c5ccccc5)(c5ccccc5)c5ccc(C7CC8CCC7C8)cc5-6)c5ccc6c(c5)oc5ccccc56)cc4)c23)cc1 ; c1ccc(-c2cccc3cccc(-c4ccc(N(c5ccc(C6(c7ccccc7)c7ccccc7-c7cc(C8CC9CCC8C9)ccc76)cc5)c5ccc6oc7ccccc7c6c5)cc4)c23)cc1 ; CC1(C)c2ccccc2-c2cc(N(c3ccc(-c4cccc5cccc(-c6ccccc6)c45)cc3)c3ccc4c(c3)C3(c5ccccc5-c5ccccc53)c3cc(C5CC6CCC5C6)ccc3-4)ccc21]

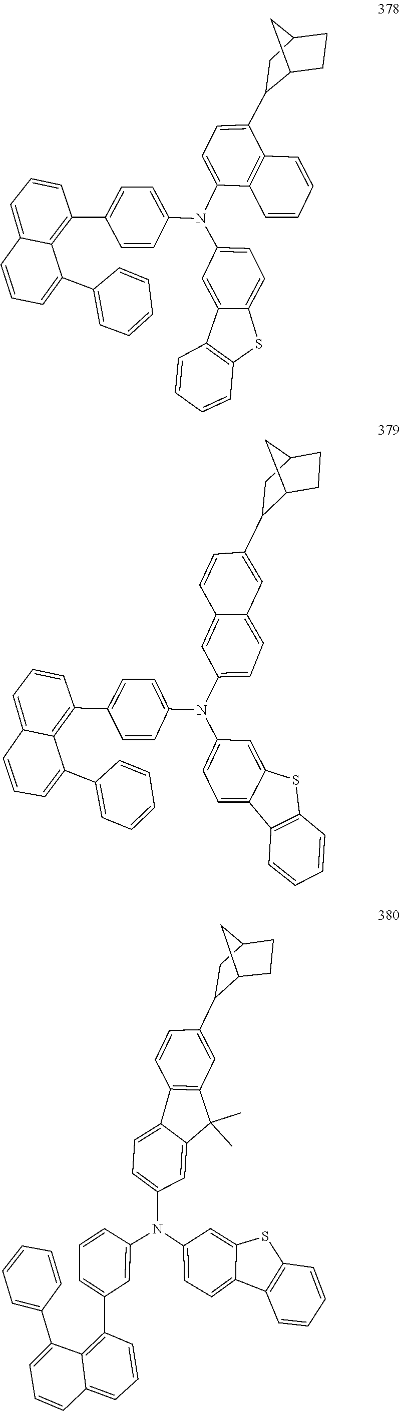 [SMILES: c1ccc(-c2cccc3cccc(-c4ccc(N(c5ccc6sc7ccccc7c6c5)c5ccc(C6CC7CCC6C7)c6ccccc56)cc4)c23)cc1 ; c1ccc(-c2cccc3cccc(-c4ccc(N(c5ccc6cc(C7CC8CCC7C8)ccc6c5)c5ccc6c(c5)sc5ccccc56)cc4)c23)cc1 ; CC1(C)c2cc(C3CC4CCC3C4)ccc2-c2ccc(N(c3cccc(-c4cccc5cccc(-c6ccccc6)c45)c3)c3ccc4c(c3)sc3ccccc34)cc21]

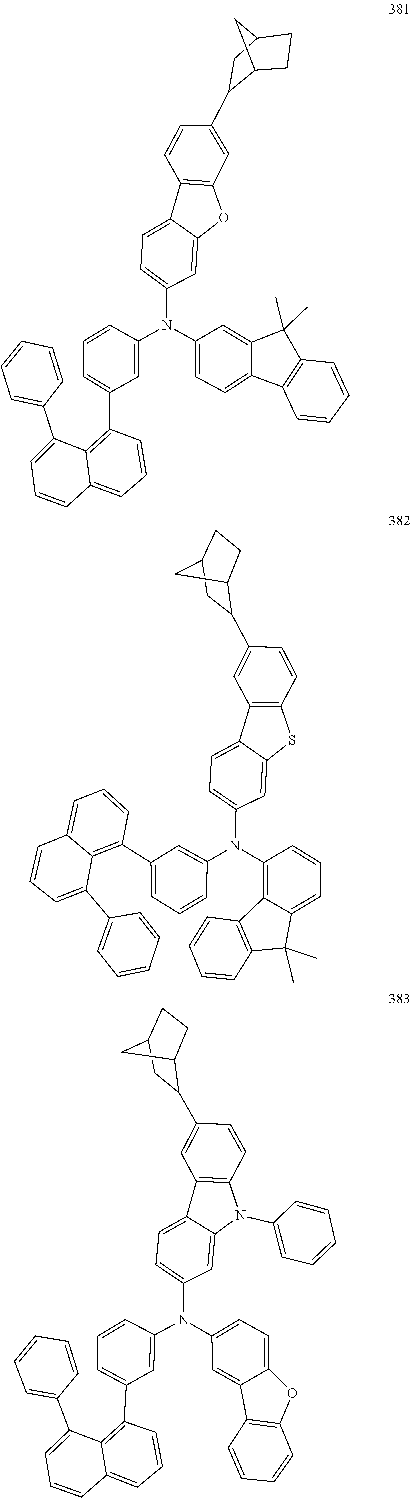

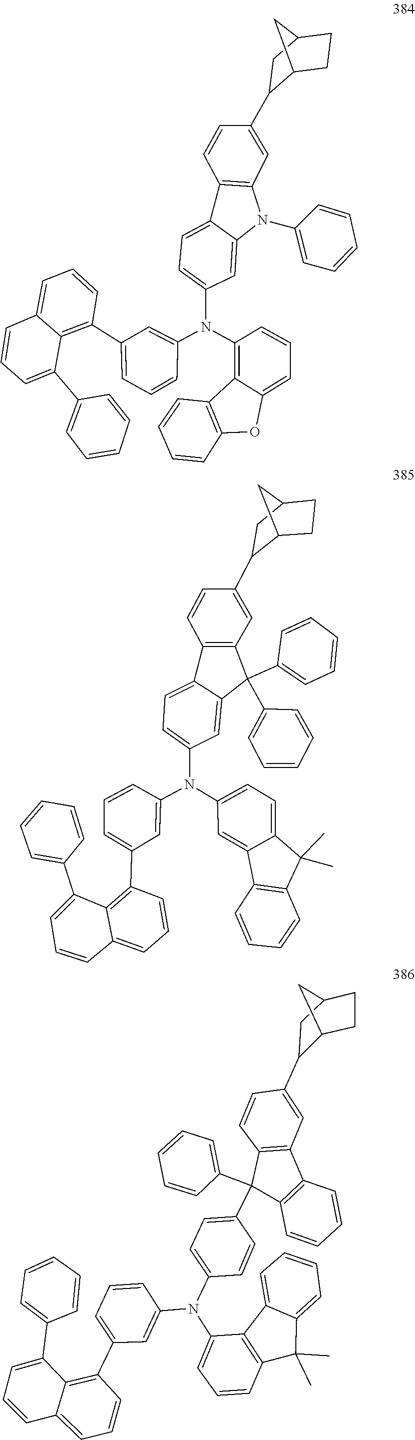

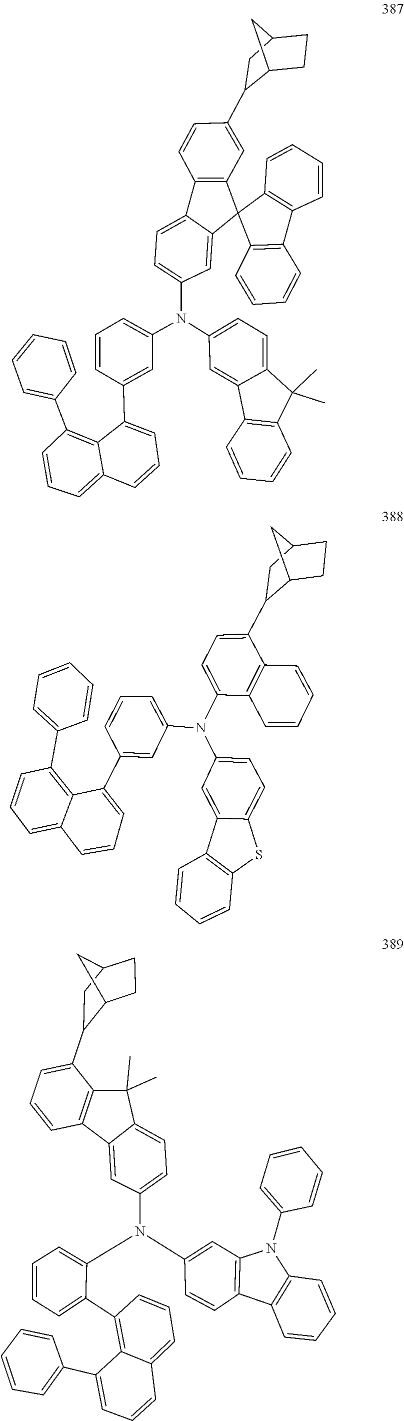

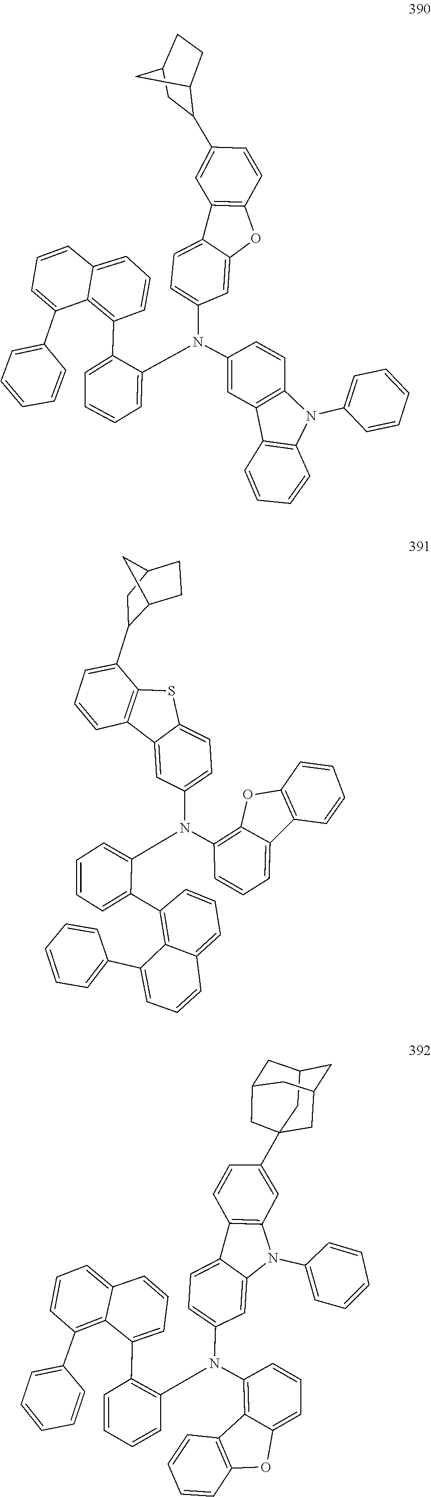 [SMILES: c1ccc(-c2cccc3cccc(-c4ccccc4N(c4ccc5c(c4)oc4ccc(C6CC7CCC6C7)cc45)c4ccc5c(c4)c4ccccc4n5-c4ccccc4)c23)cc1 ; c1ccc(-c2cccc3cccc(-c4ccccc4N(c4ccc5sc6c(C7CC8CCC7C8)cccc6c5c4)c4cccc5c4oc4ccccc45)c23)cc1 ; c1ccc(-c2cccc3cccc(-c4ccccc4N(c4ccc5c6ccc(C78CC9CC(CC(C9)C7)C8)cc6n(-c6ccccc6)c5c4)c4cccc5oc6ccccc6c45)c23)cc1]

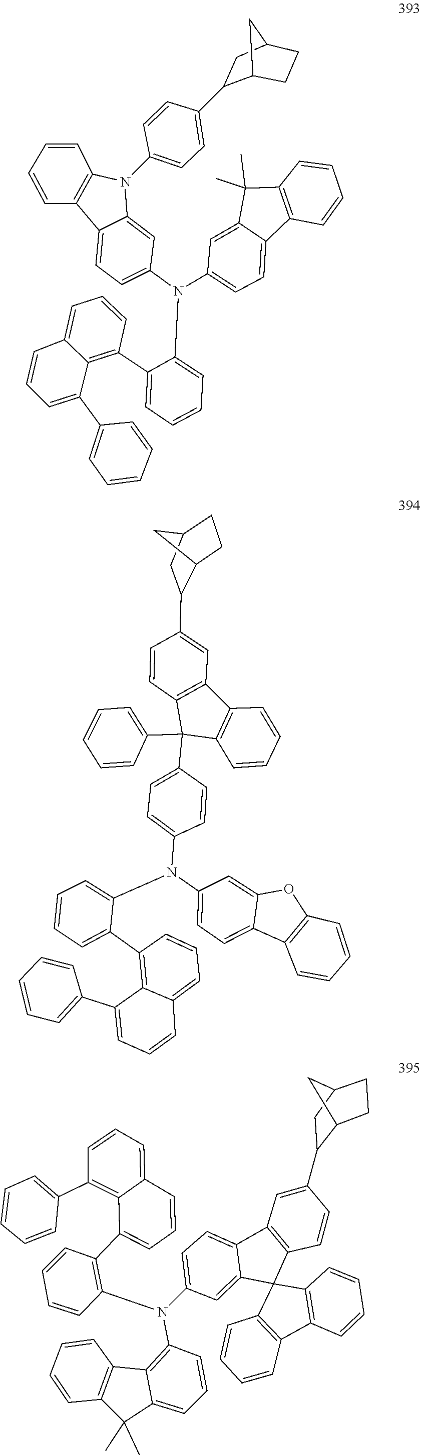 [SMILES: CC1(C)c2ccccc2-c2ccc(N(c3ccc4c5ccccc5n(-c5ccc(C6CC7CCC6C7)cc5)c4c3)c3ccccc3-c3cccc4cccc(-c5ccccc5)c34)cc21 ; c1ccc(-c2cccc3cccc(-c4ccccc4N(c4ccc(C5(c6ccccc6)c6ccccc6-c6cc(C7CC8CCC7C8)ccc65)cc4)c4ccc5c(c4)oc4ccccc45)c23)cc1 ; CC1(C)c2ccccc2-c2c(N(c3ccc4c(c3)C3(c5ccccc5-c5ccccc53)c3ccc(C5CC6CCC5C6)cc3-4)c3ccccc3-c3cccc4cccc(-c5ccccc5)c34)cccc21]

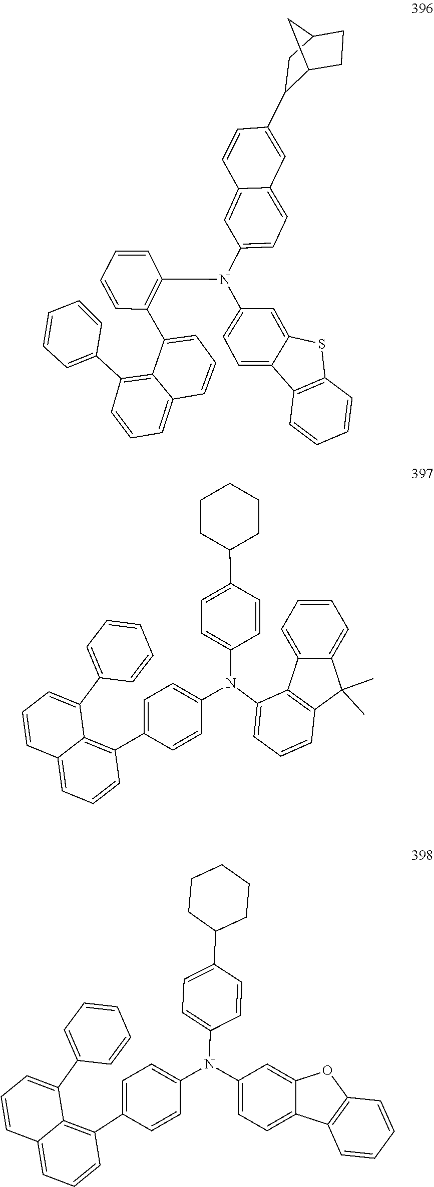 [SMILES: c1ccc(-c2cccc3cccc(-c4ccccc4N(c4ccc5cc(C6CC7CCC6C7)ccc5c4)c4ccc5c(c4)sc4ccccc45)c23)cc1 ; CC1(C)c2ccccc2-c2c(N(c3ccc(-c4cccc5cccc(-c6ccccc6)c45)cc3)c3ccc(C4CCCCC4)cc3)cccc21 ; c1ccc(-c2cccc3cccc(-c4ccc(N(c5ccc(C6CCCCC6)cc5)c5ccc6c(c5)oc5ccccc56)cc4)c23)cc1]

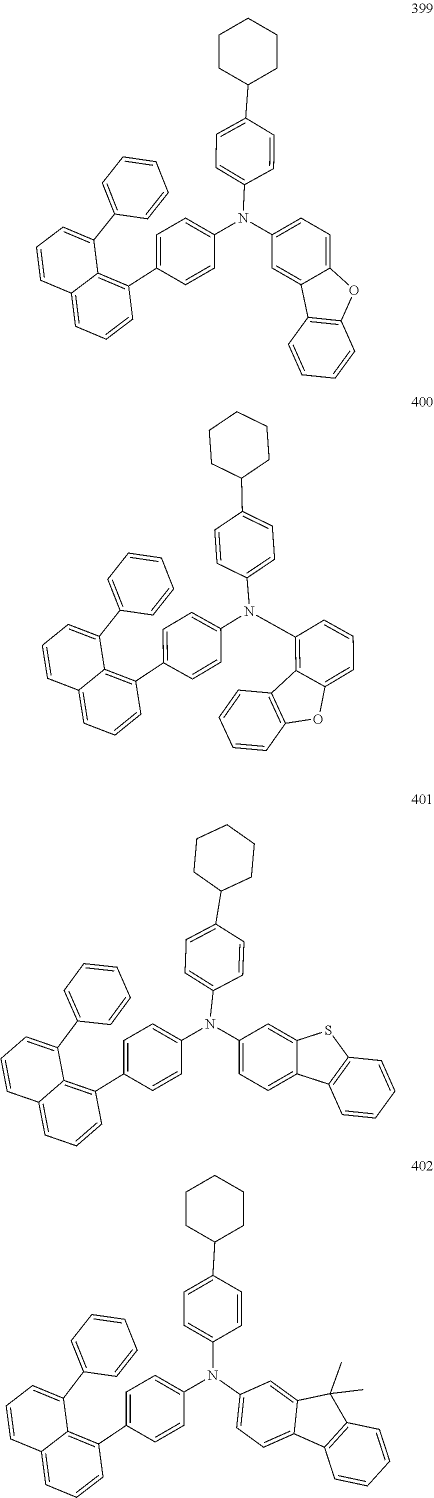 [SMILES: c1ccc(-c2cccc3cccc(-c4ccc(N(c5ccc(C6CCCCC6)cc5)c5ccc6oc7ccccc7c6c5)cc4)c23)cc1 ; c1ccc(-c2cccc3cccc(-c4ccc(N(c5ccc(C6CCCCC6)cc5)c5cccc6oc7ccccc7c56)cc4)c23)cc1 ; c1ccc(-c2cccc3cccc(-c4ccc(N(c5ccc(C6CCCCC6)cc5)c5ccc6c(c5)sc5ccccc56)cc4)c23)cc1 ; CC1(C)c2ccccc2-c2ccc(N(c3ccc(-c4cccc5cccc(-c6ccccc6)c45)cc3)c3ccc(C4CCCCC4)cc3)cc21]

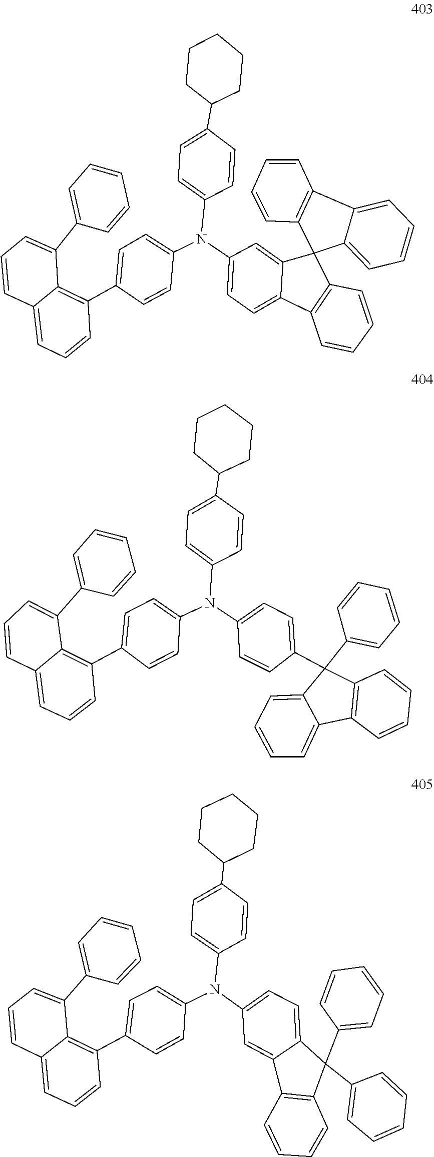 [SMILES: c1ccc(-c2cccc3cccc(-c4ccc(N(c5ccc(C6CCCCC6)cc5)c5ccc6c(c5)C5(c7ccccc7-c7ccccc75)c5ccccc5-6)cc4)c23)cc1 ; c1ccc(-c2cccc3cccc(-c4ccc(N(c5ccc(C6CCCCC6)cc5)c5ccc(C6(c7ccccc7)c7ccccc7-c7ccccc76)cc5)cc4)c23)cc1 ; c1ccc(-c2cccc3cccc(-c4ccc(N(c5ccc(C6CCCCC6)cc5)c5ccc6c(c5)-c5ccccc5C6(c5ccccc5)c5ccccc5)cc4)c23)cc1]

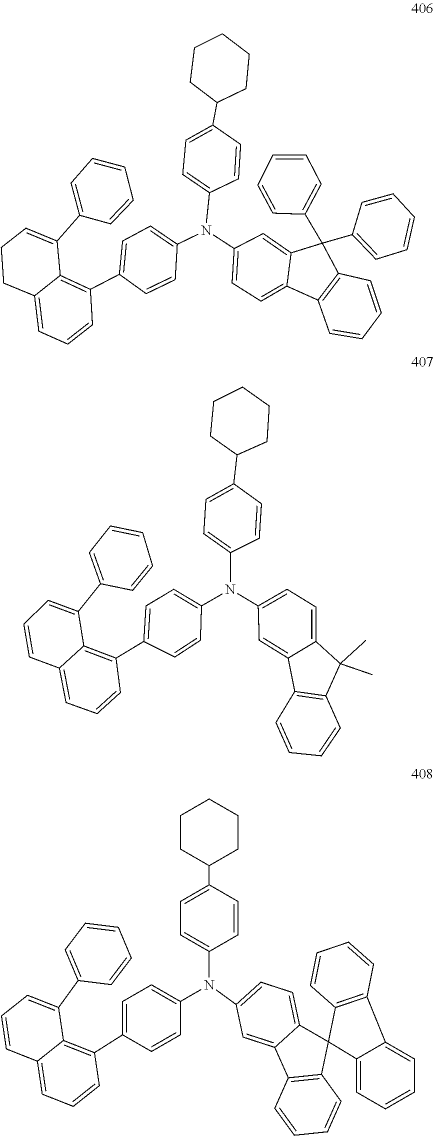 [SMILES: C1=C(c2ccccc2)c2c(cccc2-c2ccc(N(c3ccc(C4CCCCC4)cc3)c3ccc4c(c3)C(c3ccccc3)(c3ccccc3)c3ccccc3-4)cc2)CC1 ; CC1(C)c2ccccc2-c2cc(N(c3ccc(-c4cccc5cccc(-c6ccccc6)c45)cc3)c3ccc(C4CCCCC4)cc3)ccc21 ; c1ccc(-c2cccc3cccc(-c4ccc(N(c5ccc(C6CCCCC6)cc5)c5ccc6c(c5)-c5ccccc5C65c6ccccc6-c6ccccc65)cc4)c23)cc1]

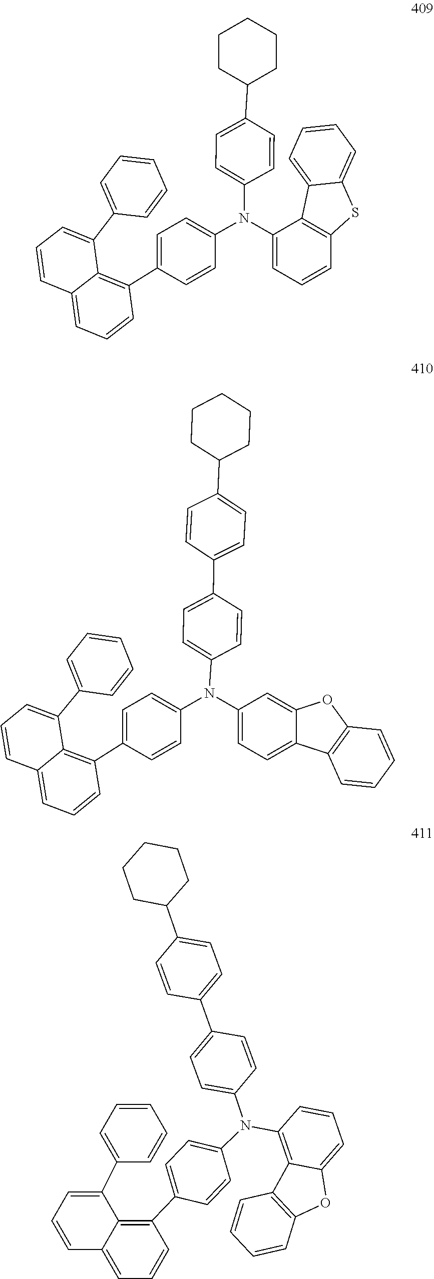 [SMILES: c1ccc(-c2cccc3cccc(-c4ccc(N(c5ccc(C6CCCCC6)cc5)c5cccc6sc7ccccc7c56)cc4)c23)cc1 ; c1ccc(-c2cccc3cccc(-c4ccc(N(c5ccc(-c6ccc(C7CCCCC7)cc6)cc5)c5ccc6c(c5)oc5ccccc56)cc4)c23)cc1 ; c1ccc(-c2cccc3cccc(-c4ccc(N(c5ccc(-c6ccc(C7CCCCC7)cc6)cc5)c5cccc6oc7ccccc7c56)cc4)c23)cc1]

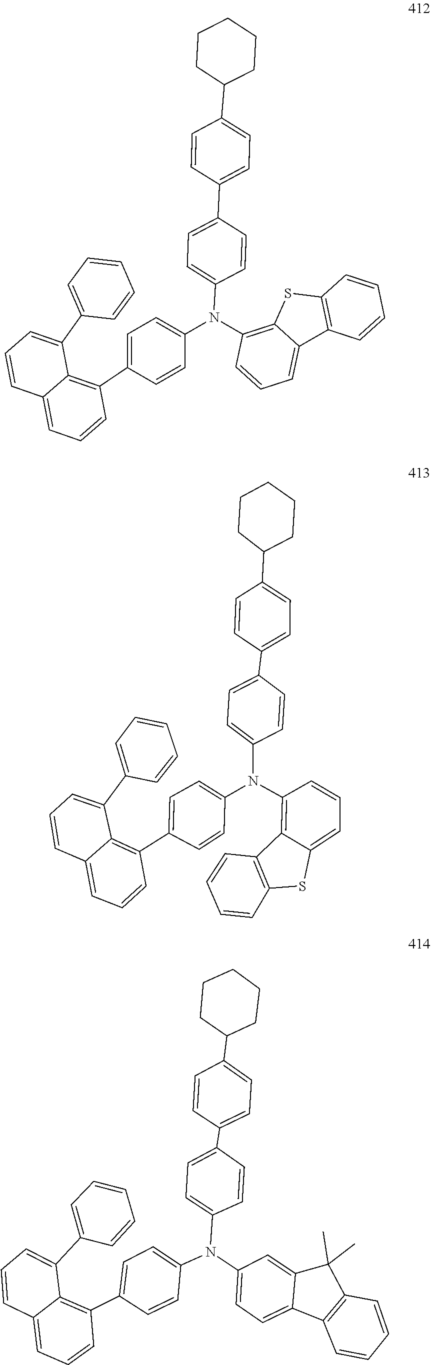 [SMILES: c1ccc(-c2cccc3cccc(-c4ccc(N(c5ccc(-c6ccc(C7CCCCC7)cc6)cc5)c5cccc6c5sc5ccccc56)cc4)c23)cc1 ; c1ccc(-c2cccc3cccc(-c4ccc(N(c5ccc(-c6ccc(C7CCCCC7)cc6)cc5)c5cccc6sc7ccccc7c56)cc4)c23)cc1 ; CC1(C)c2ccccc2-c2ccc(N(c3ccc(-c4ccc(C5CCCCC5)cc4)cc3)c3ccc(-c4cccc5cccc(-c6ccccc6)c45)cc3)cc21]

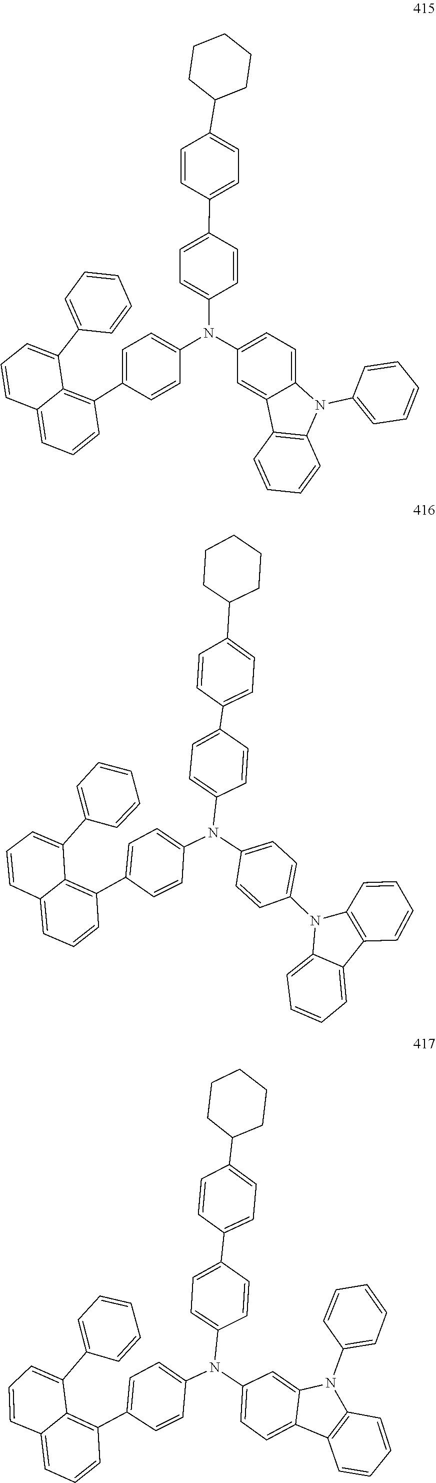 [SMILES: c1ccc(-c2cccc3cccc(-c4ccc(N(c5ccc(-c6ccc(C7CCCCC7)cc6)cc5)c5ccc6c(c5)c5ccccc5n6-c5ccccc5)cc4)c23)cc1 ; c1ccc(-c2cccc3cccc(-c4ccc(N(c5ccc(-c6ccc(C7CCCCC7)cc6)cc5)c5ccc(-n6c7ccccc7c7ccccc76)cc5)cc4)c23)cc1 ; c1ccc(-c2cccc3cccc(-c4ccc(N(c5ccc(-c6ccc(C7CCCCC7)cc6)cc5)c5ccc6c7ccccc7n(-c7ccccc7)c6c5)cc4)c23)cc1]

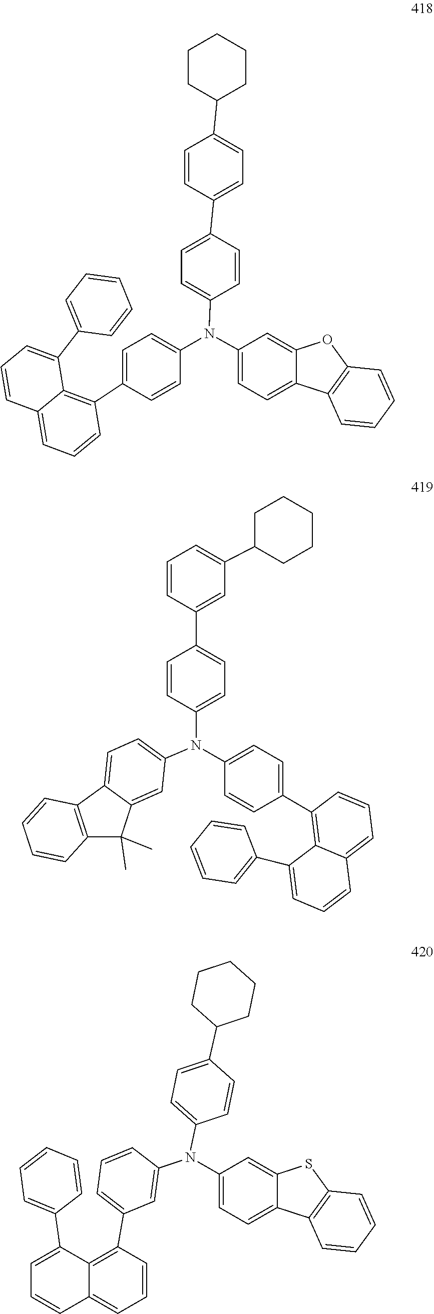 [SMILES: c1ccc(-c2cccc3cccc(-c4ccc(N(c5ccc(-c6ccc(C7CCCCC7)cc6)cc5)c5ccc6c(c5)oc5ccccc56)cc4)c23)cc1 ; CC1(C)c2ccccc2-c2ccc(N(c3ccc(-c4cccc(C5CCCCC5)c4)cc3)c3ccc(-c4cccc5cccc(-c6ccccc6)c45)cc3)cc21 ; c1ccc(-c2cccc3cccc(-c4cccc(N(c5ccc(C6CCCCC6)cc5)c5ccc6c(c5)sc5ccccc56)c4)c23)cc1]

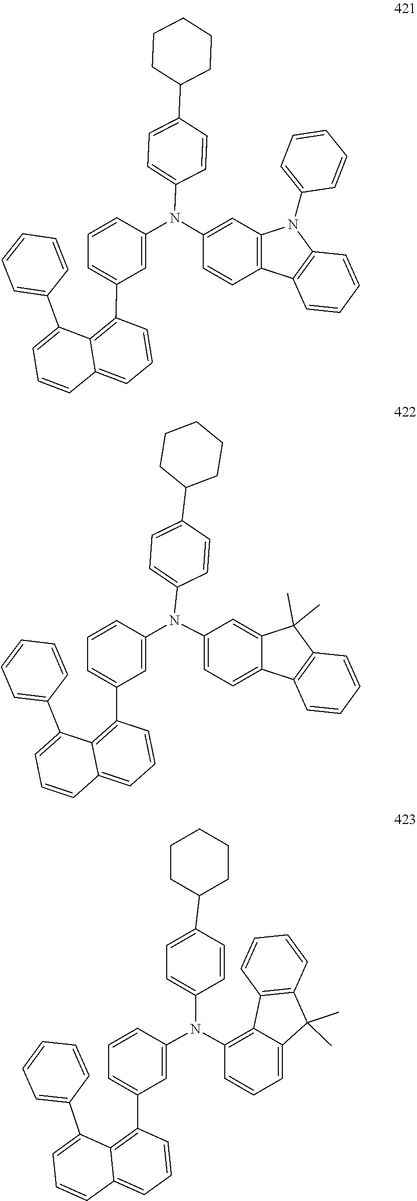 [SMILES: c1ccc(-c2cccc3cccc(-c4cccc(N(c5ccc(C6CCCCC6)cc5)c5ccc6c7ccccc7n(-c7ccccc7)c6c5)c4)c23)cc1 ; CC1(C)c2ccccc2-c2ccc(N(c3ccc(C4CCCCC4)cc3)c3cccc(-c4cccc5cccc(-c6ccccc6)c45)c3)cc21 ; CC1(C)c2ccccc2-c2c(N(c3ccc(C4CCCCC4)cc3)c3cccc(-c4cccc5cccc(-c6ccccc6)c45)c3)cccc21]

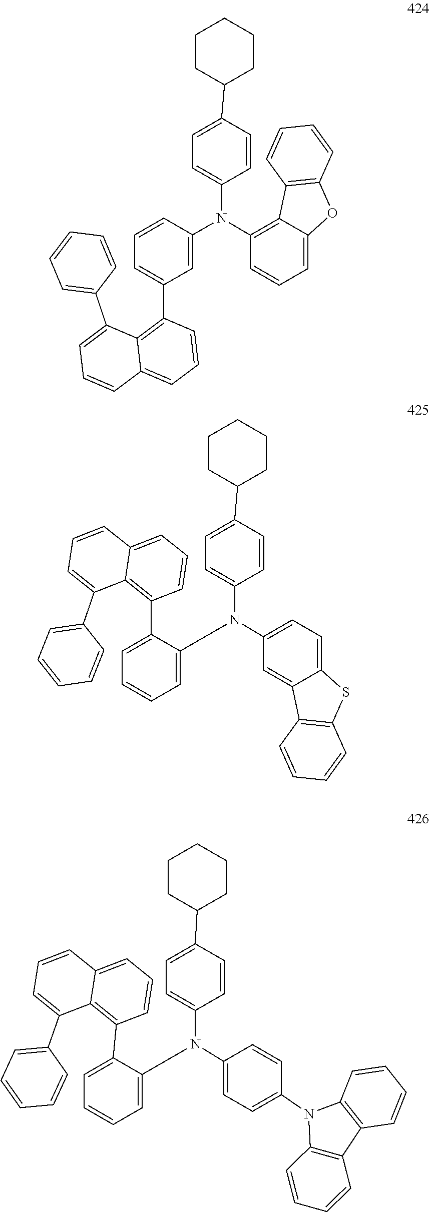 [SMILES: c1ccc(-c2cccc3cccc(-c4cccc(N(c5ccc(C6CCCCC6)cc5)c5cccc6oc7ccccc7c56)c4)c23)cc1 ; c1ccc(-c2cccc3cccc(-c4ccccc4N(c4ccc(C5CCCCC5)cc4)c4ccc5sc6ccccc6c5c4)c23)cc1 ; c1ccc(-c2cccc3cccc(-c4ccccc4N(c4ccc(C5CCCCC5)cc4)c4ccc(-n5c6ccccc6c6ccccc65)cc4)c23)cc1]

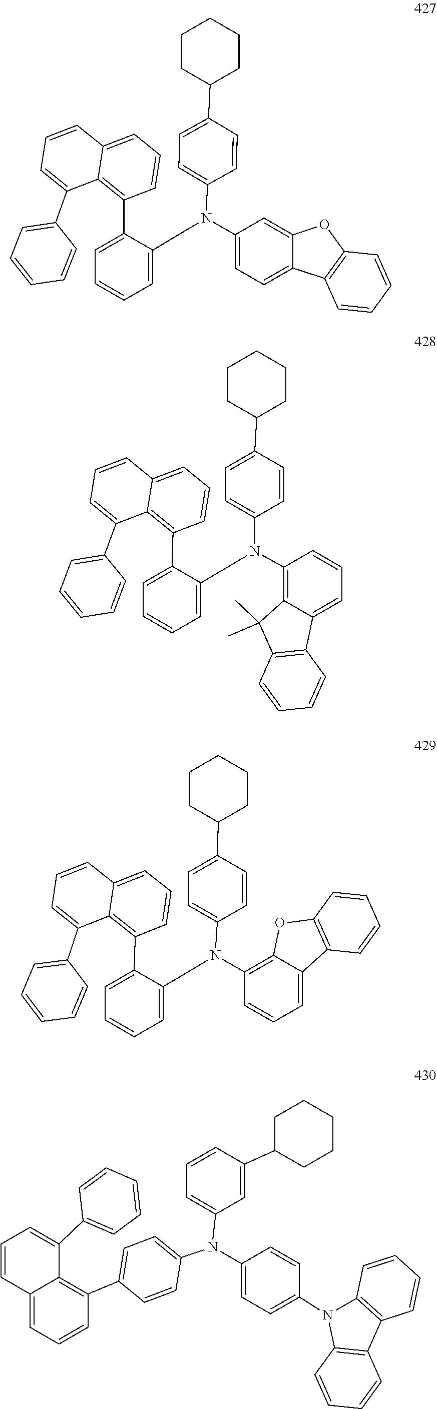 [SMILES: c1ccc(-c2cccc3cccc(-c4ccccc4N(c4ccc(C5CCCCC5)cc4)c4ccc5c(c4)oc4ccccc45)c23)cc1 ; CC1(C)c2ccccc2-c2cccc(N(c3ccc(C4CCCCC4)cc3)c3ccccc3-c3cccc4cccc(-c5ccccc5)c34)c21 ; c1ccc(-c2cccc3cccc(-c4ccccc4N(c4ccc(C5CCCCC5)cc4)c4cccc5c4oc4ccccc45)c23)cc1 ; c1ccc(-c2cccc3cccc(-c4ccc(N(c5ccc(-n6c7ccccc7c7ccccc76)cc5)c5cccc(C6CCCCC6)c5)cc4)c23)cc1]

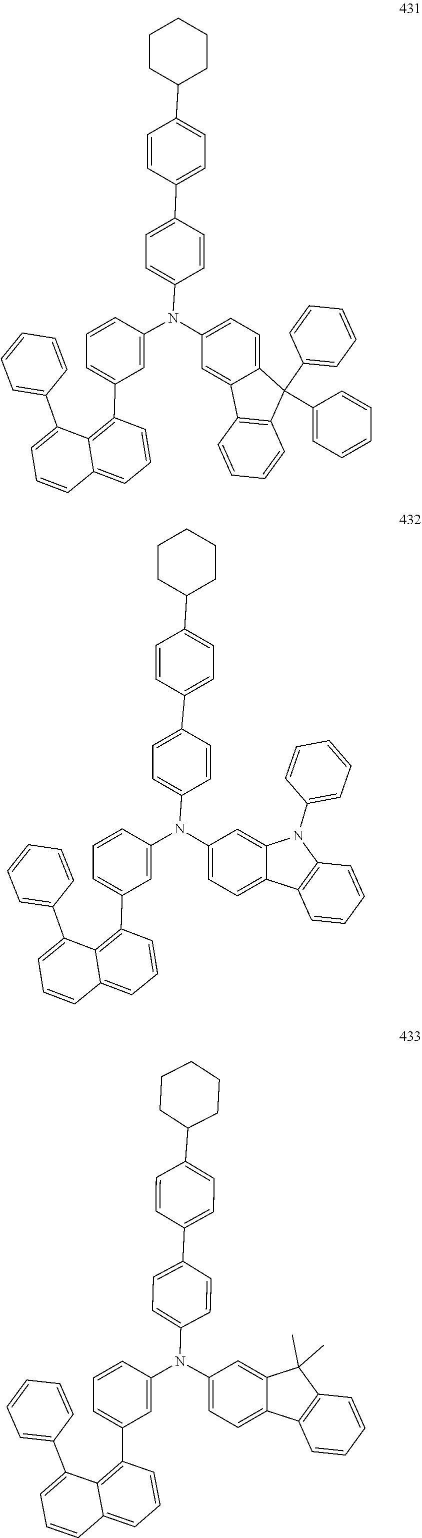 [SMILES: c1ccc(-c2cccc3cccc(-c4cccc(N(c5ccc(-c6ccc(C7CCCCC7)cc6)cc5)c5ccc6c(c5)-c5ccccc5C6(c5ccccc5)c5ccccc5)c4)c23)cc1 ; c1ccc(-c2cccc3cccc(-c4cccc(N(c5ccc(-c6ccc(C7CCCCC7)cc6)cc5)c5ccc6c7ccccc7n(-c7ccccc7)c6c5)c4)c23)cc1 ; CC1(C)c2ccccc2-c2ccc(N(c3ccc(-c4ccc(C5CCCCC5)cc4)cc3)c3cccc(-c4cccc5cccc(-c6ccccc6)c45)c3)cc21]

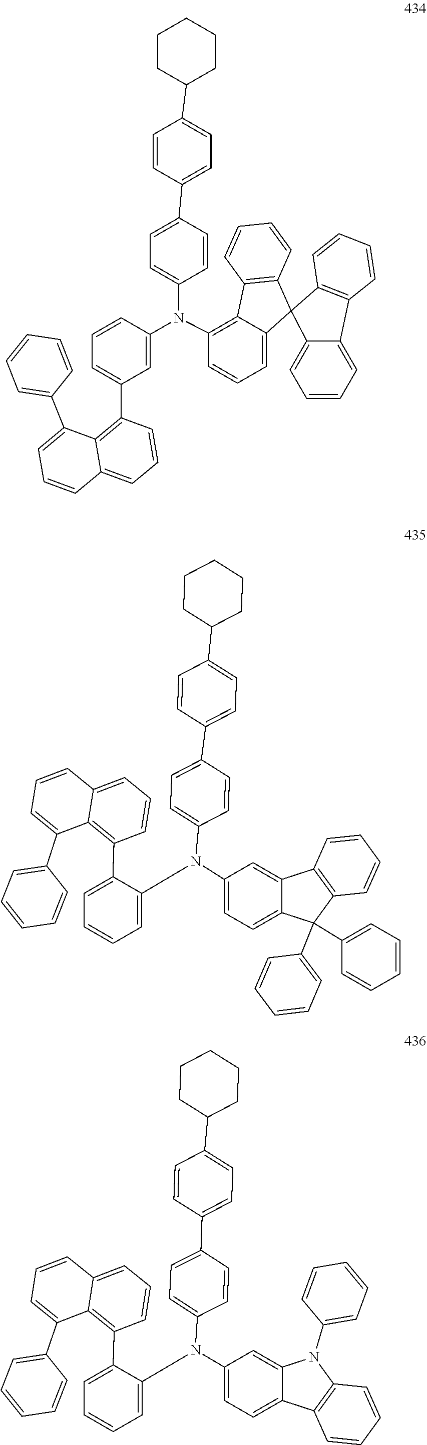 [SMILES: c1ccc(-c2cccc3cccc(-c4cccc(N(c5ccc(-c6ccc(C7CCCCC7)cc6)cc5)c5cccc6c5-c5ccccc5C65c6ccccc6-c6ccccc65)c4)c23)cc1 ; c1ccc(-c2cccc3cccc(-c4ccccc4N(c4ccc(-c5ccc(C6CCCCC6)cc5)cc4)c4ccc5c(c4)-c4ccccc4C5(c4ccccc4)c4ccccc4)c23)cc1 ; c1ccc(-c2cccc3cccc(-c4ccccc4N(c4ccc(-c5ccc(C6CCCCC6)cc5)cc4)c4ccc5c6ccccc6n(-c6ccccc6)c5c4)c23)cc1]

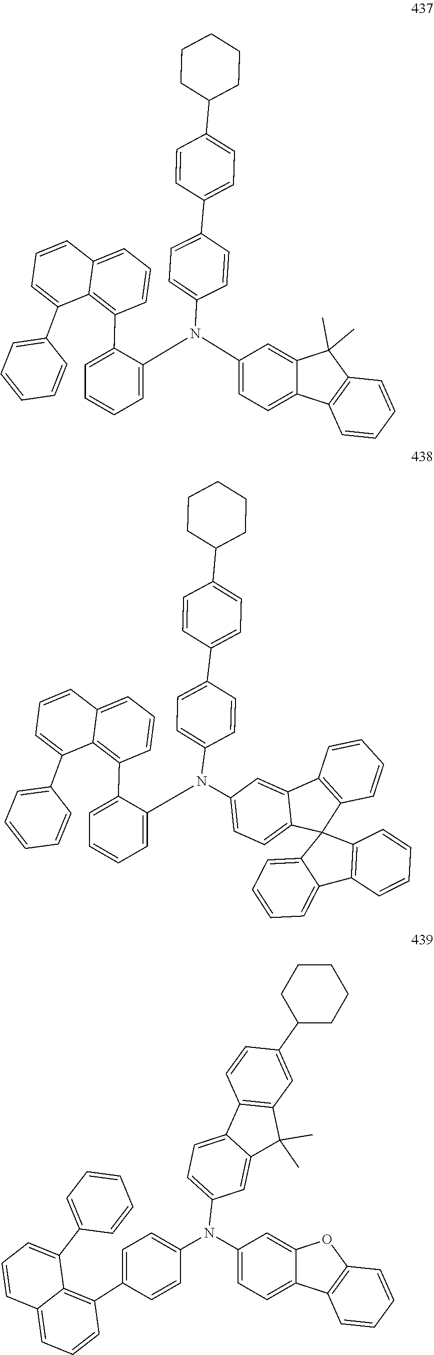 [SMILES: CC1(C)c2ccccc2-c2ccc(N(c3ccc(-c4ccc(C5CCCCC5)cc4)cc3)c3ccccc3-c3cccc4cccc(-c5ccccc5)c34)cc21 ; c1ccc(-c2cccc3cccc(-c4ccccc4N(c4ccc(-c5ccc(C6CCCCC6)cc5)cc4)c4ccc5c(c4)-c4ccccc4C54c5ccccc5-c5ccccc54)c23)cc1 ; CC1(C)c2cc(C3CCCCC3)ccc2-c2ccc(N(c3ccc(-c4cccc5cccc(-c6ccccc6)c45)cc3)c3ccc4c(c3)oc3ccccc34)cc21]

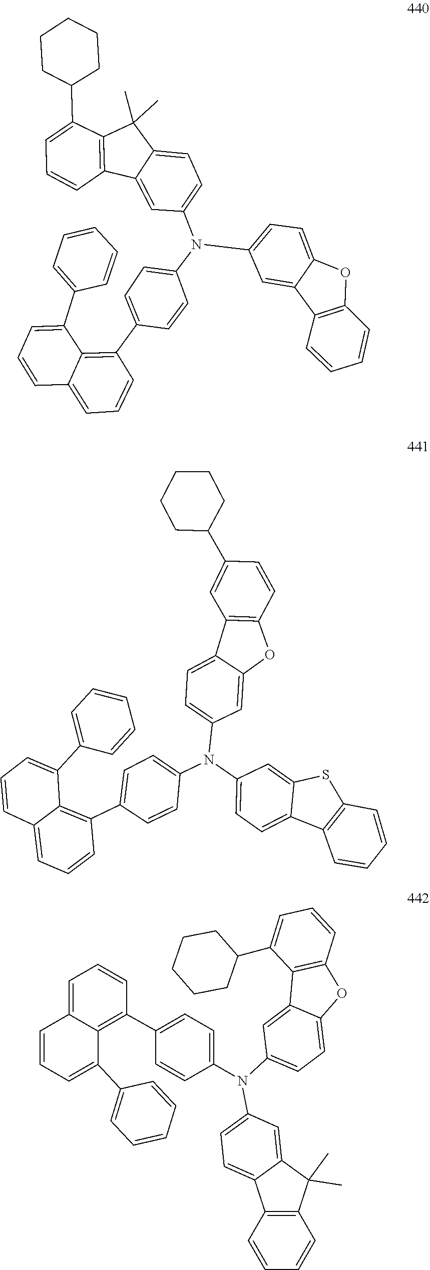 [SMILES: CC1(C)c2ccc(N(c3ccc(-c4cccc5cccc(-c6ccccc6)c45)cc3)c3ccc4oc5ccccc5c4c3)cc2-c2cccc(C3CCCCC3)c21 ; c1ccc(-c2cccc3cccc(-c4ccc(N(c5ccc6c(c5)oc5ccc(C7CCCCC7)cc56)c5ccc6c(c5)sc5ccccc56)cc4)c23)cc1 ; CC1(C)c2ccccc2-c2ccc(N(c3ccc(-c4cccc5cccc(-c6ccccc6)c45)cc3)c3ccc4oc5cccc(C6CCCCC6)c5c4c3)cc21]

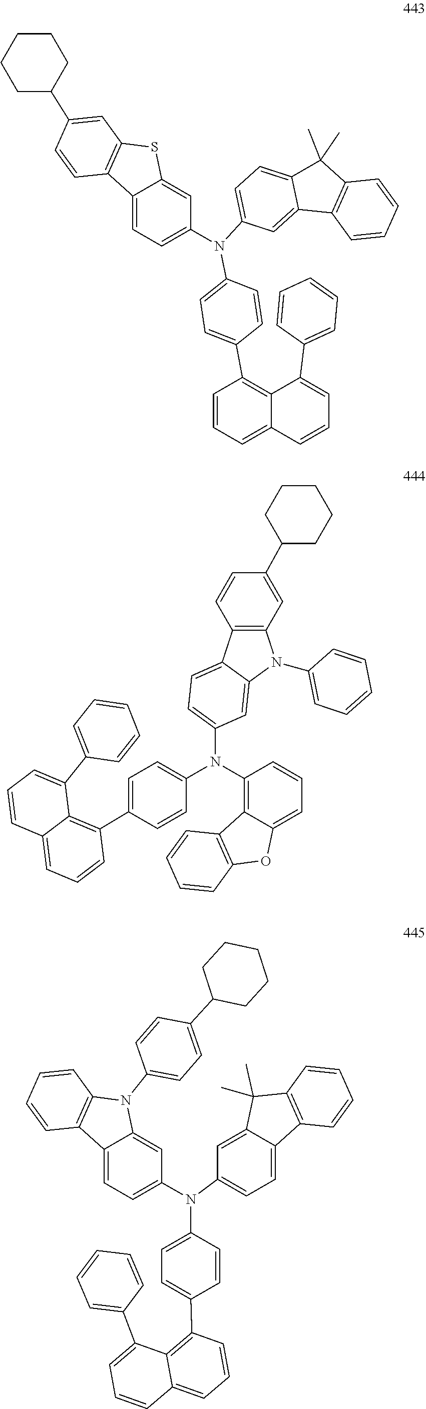

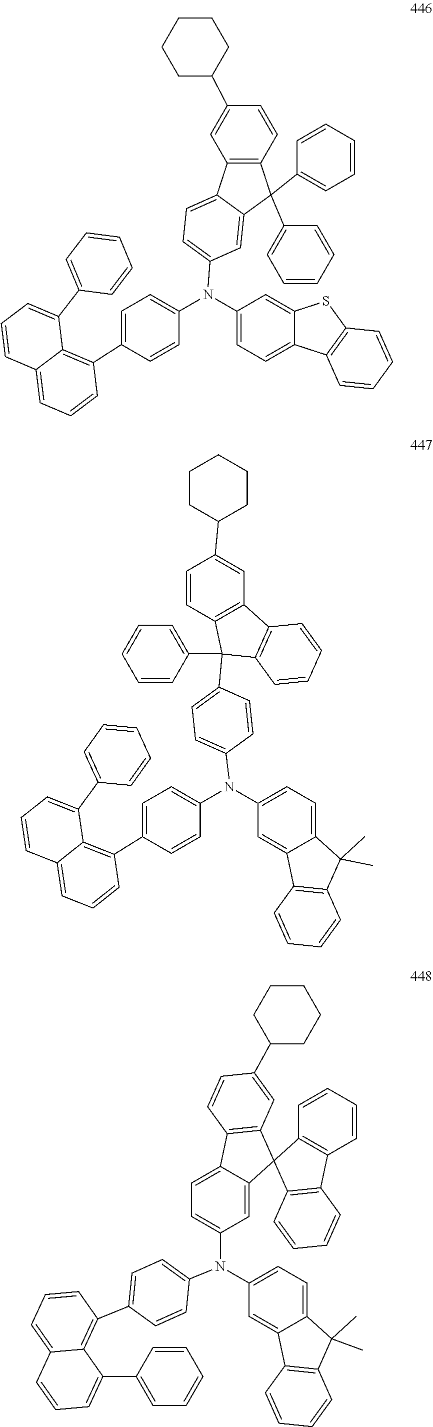

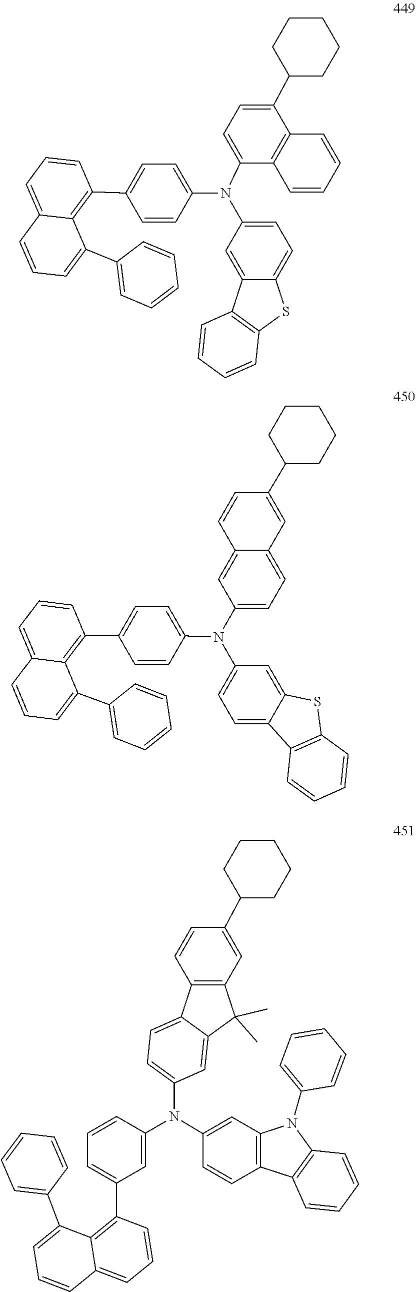 [SMILES: c1ccc(-c2cccc3cccc(-c4ccc(N(c5ccc6sc7ccccc7c6c5)c5ccc(C6CCCCC6)c6ccccc56)cc4)c23)cc1 ; c1ccc(-c2cccc3cccc(-c4ccc(N(c5ccc6cc(C7CCCCC7)ccc6c5)c5ccc6c(c5)sc5ccccc56)cc4)c23)cc1 ; CC1(C)c2cc(C3CCCCC3)ccc2-c2ccc(N(c3cccc(-c4cccc5cccc(-c6ccccc6)c45)c3)c3ccc4c5ccccc5n(-c5ccccc5)c4c3)cc21]

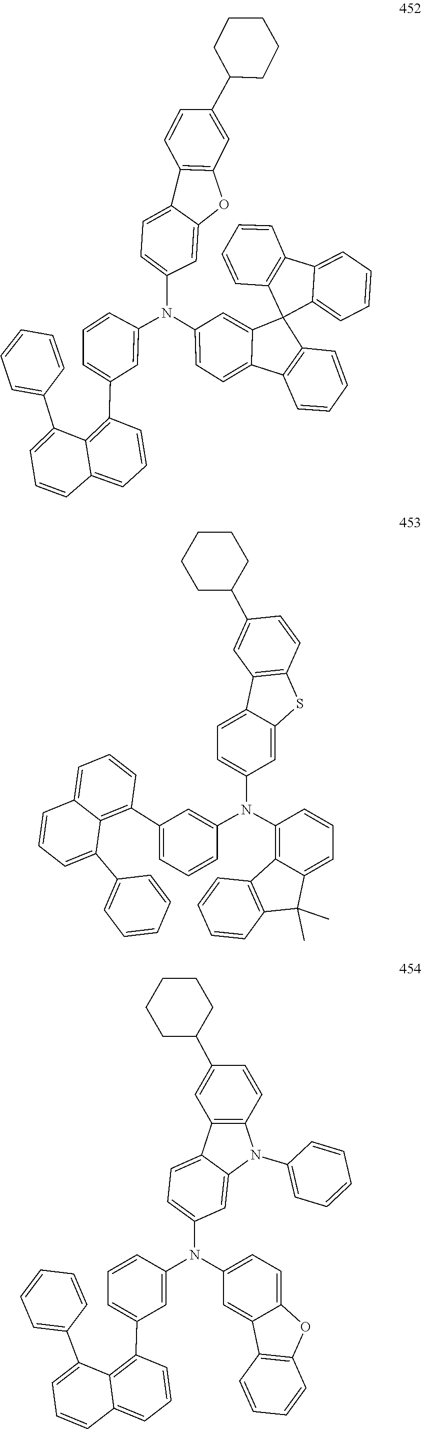

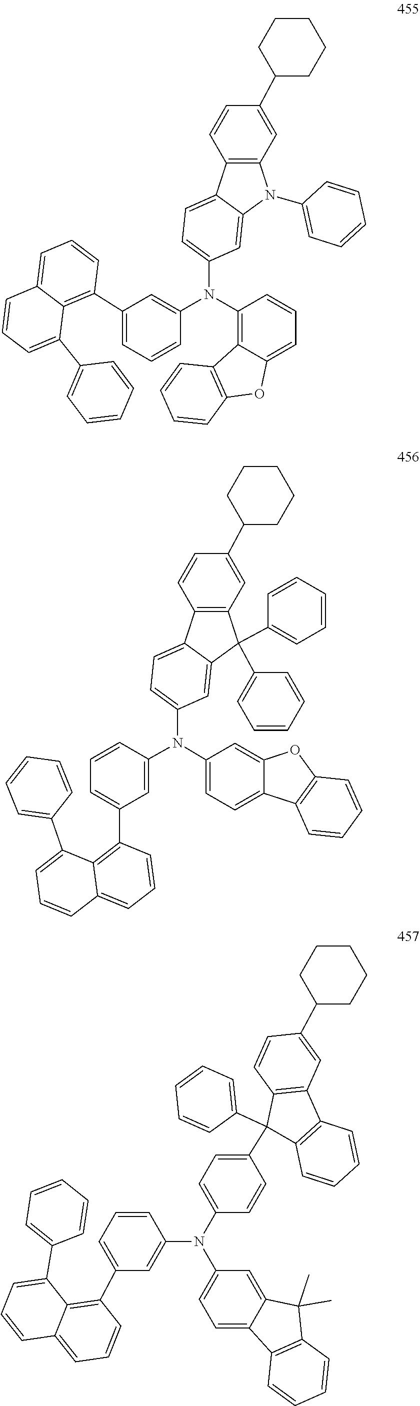 [SMILES: c1ccc(-c2cccc3cccc(-c4cccc(N(c5ccc6c7ccc(C8CCCCC8)cc7n(-c7ccccc7)c6c5)c5cccc6oc7ccccc7c56)c4)c23)cc1 ; c1ccc(-c2cccc3cccc(-c4cccc(N(c5ccc6c(c5)C(c5ccccc5)(c5ccccc5)c5cc(C7CCCCC7)ccc5-6)c5ccc6c(c5)oc5ccccc56)c4)c23)cc1 ; CC1(C)c2ccccc2-c2ccc(N(c3ccc(C4(c5ccccc5)c5ccccc5-c5cc(C6CCCCC6)ccc54)cc3)c3cccc(-c4cccc5cccc(-c6ccccc6)c45)c3)cc21]

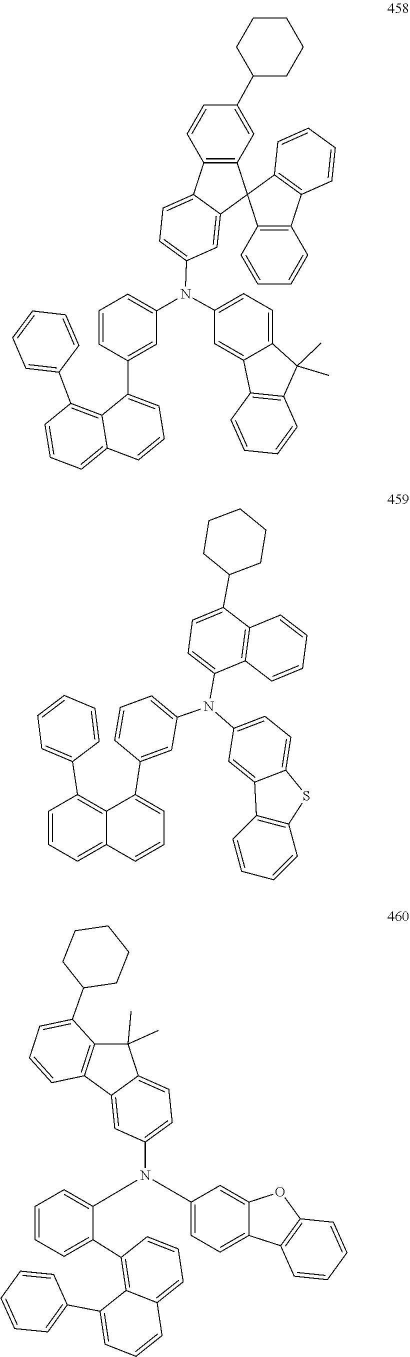 [SMILES: CC1(C)c2ccccc2-c2cc(N(c3cccc(-c4cccc5cccc(-c6ccccc6)c45)c3)c3ccc4c(c3)C3(c5ccccc5-c5ccccc53)c3cc(C5CCCCC5)ccc3-4)ccc21 ; c1ccc(-c2cccc3cccc(-c4cccc(N(c5ccc6sc7ccccc7c6c5)c5ccc(C6CCCCC6)c6ccccc56)c4)c23)cc1 ; CC1(C)c2ccc(N(c3ccc4c(c3)oc3ccccc34)c3ccccc3-c3cccc4cccc(-c5ccccc5)c34)cc2-c2cccc(C3CCCCC3)c21]

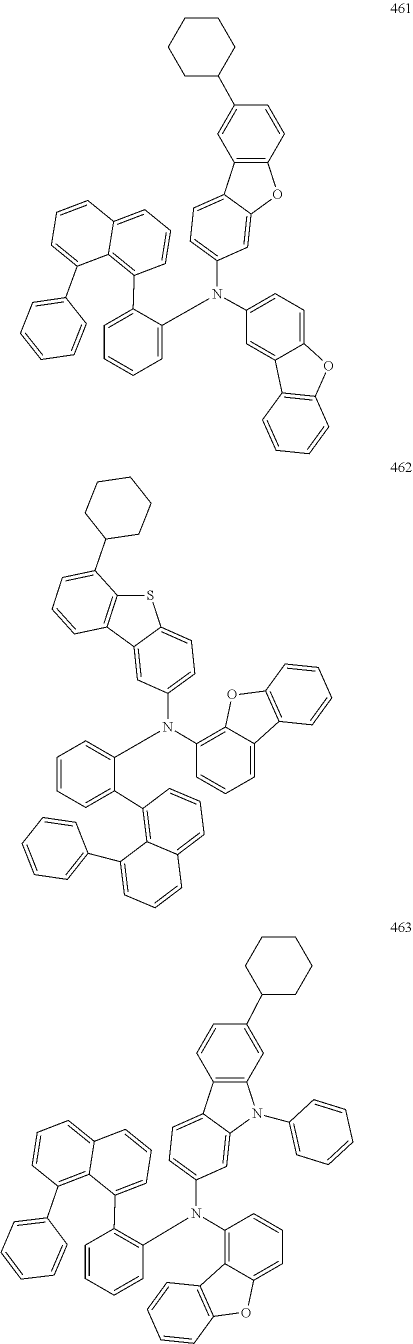 [SMILES: c1ccc(-c2cccc3cccc(-c4ccccc4N(c4ccc5c(c4)oc4ccc(C6CCCCC6)cc45)c4ccc5oc6ccccc6c5c4)c23)cc1 ; c1ccc(-c2cccc3cccc(-c4ccccc4N(c4ccc5sc6c(C7CCCCC7)cccc6c5c4)c4cccc5c4oc4ccccc45)c23)cc1 ; c1ccc(-c2cccc3cccc(-c4ccccc4N(c4ccc5c6ccc(C7CCCCC7)cc6n(-c6ccccc6)c5c4)c4cccc5oc6ccccc6c45)c23)cc1]

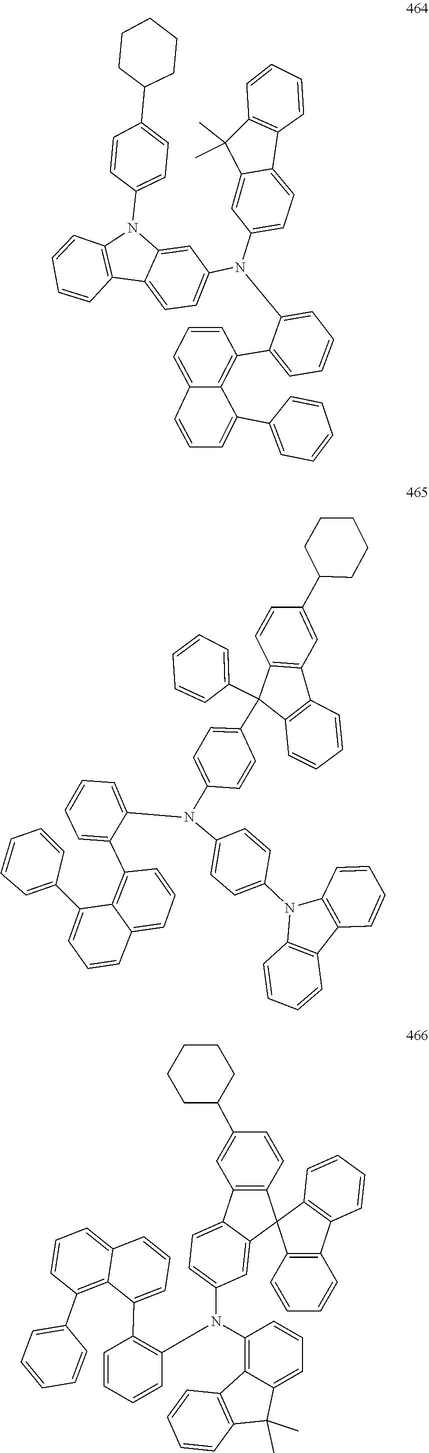 [SMILES: CC1(C)c2ccccc2-c2ccc(N(c3ccc4c5ccccc5n(-c5ccc(C6CCCCC6)cc5)c4c3)c3ccccc3-c3cccc4cccc(-c5ccccc5)c34)cc21 ; c1ccc(-c2cccc3cccc(-c4ccccc4N(c4ccc(-n5c6ccccc6c6ccccc65)cc4)c4ccc(C5(c6ccccc6)c6ccccc6-c6cc(C7CCCCC7)ccc65)cc4)c23)cc1 ; CC1(C)c2ccccc2-c2c(N(c3ccc4c(c3)C3(c5ccccc5-c5ccccc53)c3ccc(C5CCCCC5)cc3-4)c3ccccc3-c3cccc4cccc(-c5ccccc5)c34)cccc21]

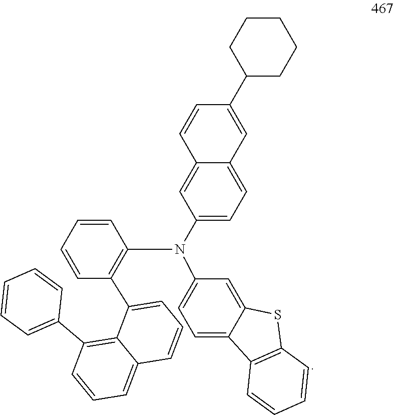 [SMILES: c1ccc(-c2cccc3cccc(-c4ccccc4N(c4ccc5cc(C6CCCCC6)ccc5c4)c4ccc5c(c4)sc4ccccc45)c23)cc1]

13. An electronic component, comprising an anode and a cathode which are oppositely disposed, and a functional layer disposed between the anode and the cathode; wherein the functional layer comprises the organic compound according to claim 1.

14. The electronic component according to claim 13, wherein the functional layer comprises an electron blocking layer, and the electron blocking layer comprises the organic compound.

15. An electronic device, comprising the electronic component according to claim 13.

16. The electronic component according to claim 14, wherein the electronic component is an organic electroluminescent device.
